(19) United States
(12) Patent Application Publication
(10) Pub. No.: US 2026/0250396 A1
(43) Pub. Date: Aug. 27, 2026

(54) THERAPIES WITH ANTI-GAL3 ANTIBODIES

(71) Applicant: TrueBinding, Inc., Santa Clara, CA (US)

(72) Inventors: Dongxu Sun, Palo Alto, CA (US); Suhail Rasool, Foster City, CA (US); George M. Haig, Melbourne, FL (US); Weimin Ni, Palo Alto, CA (US); Apurva Chandalia, Union City, CA (US)

(21) Appl. No.: 19/116,297

(22) PCT Filed: Sep. 27, 2023
(86) PCT No.: PCT/US2023/075269

§ 371 Date: Mar. 27, 2025

Related U.S. Application Data
(60) Provisional application No. 63/377,475, filed on Sep. 28, 2022.
(60) Provisional application No. 63/384,191, filed on Nov. 17, 2022.
(60) Provisional application No. 63/385,133, filed on Nov. 28, 2022.
(60) Provisional application No. 63/483,217, filed on Feb. 3, 2023.
(60) Provisional application No. 63/483,730, filed on Feb. 7, 2023.
(60) Provisional application No. 63/491,245, filed on Mar. 20, 2023.
(60) Provisional application No. 63/501,626, filed on May 11, 2023.

(51) Int. Cl. C07K 16/28; A61K 39/00; A61P 25/28

(52) U.S. Cl. CPC C07K 16/2851; A61P 25/28; A61K 2039/505; A61K 2039/54; A61K 2039/545

(57) ABSTRACT

Provided herein are therapies involving the use of antibodies that bind to Gal-3. Such therapies can include, but are not limited to, disorders such as Alzheimer's disease. Such therapies can also include treatments focused on increasing a subject's test scores under a variety of metrics.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### CROSS-REFERENCE TO RELATED APPLICATIONS

This application claims the benefit of priority of U.S. Provisional Patent Application No. 63/377,475, filed Sep. 28, 2022; U.S. Provisional Patent Application No. 63/384,191, filed Nov. 17, 2022; U.S. Provisional Patent Application No. 63/385,133, filed Nov. 28, 2022; U.S. Provisional Patent Application No. 63/483,217, filed Feb. 3, 2023; U.S. Provisional Patent Application No. 63/483,730, filed Feb. 7, 2023; U.S. Provisional Patent Application No. 63/491,245, filed Mar. 20, 2023; U.S. Provisional Patent Application No. 63/501,626, filed May 11, 2023; which are hereby expressly incorporated by reference in their entireties, including any appendices filed therewith.

### REFERENCE TO SEQUENCE LISTING

The present application is being filed along with a Sequence Listing in electronic format. The Sequence Listing is provided as a file entitled IMMUT034WOSEQLIST.XML, which was created and last modified on Sep. 26, 2023, which is 2,97976,431 bytes in size. The information in the electronic Sequence Listing is hereby incorporated by reference in its entirety.

### FIELD

Aspects of the present disclosure relate generally to therapies involving the use of antibodies that bind to Gal-3.

### BACKGROUND

Galectin-3 (Gal3, GAL3) is a lectin, or a carbohydrate-binding protein, with specificity towards beta-galactosides. In human cells, Gal3 is expressed and can be found in the nucleus, cytoplasm, cell surface, and in the extracellular space. Gal3 recognizes and interacts with beta-galactose conjugates on various proteins.

### SUMMARY

Some embodiments provided herein are described by way of the following provided embodiments and also provided as possible combinations or overlapping embodiments:

In some embodiments, a method of increasing a subject's Mini Mental State Examination (MMSE) score, the method comprising: identifying a subject as likely to benefit from an increased MMSE score, and administering a therapeutically effective amount of an antibody, wherein the antibody binds to Gal3, is disclosed.

In some embodiments, a method of increasing a subject's Mini Mental State Examination (MMSE) score, the method comprising: identifying a subject as having Alzheimer's disease (AD), and administering a therapeutically effective amount of an antibody, wherein the antibody binds to Gal3, is disclosed.

In some embodiments, a method of increasing a subject's Mini Mental State Examination (MMSE) score, the method comprising: identifying a subject as having dementia, and administering a therapeutically effective amount of an antibody, wherein the antibody binds to Gal3, is disclosed.

In some embodiments, a method of increasing a subject's Mini Mental State Examination (MMSE) score, the method comprising: identifying a subject as likely to benefit from an increased MMSE score, and administering 420 mg of an anti-Gal3 antibody, wherein the antibody is administered once weekly for 5 weeks, is disclosed.

In some embodiments, a method of increasing a subject's Mini Mental State Examination (MMSE) score, the method comprising: identifying a subject as likely to benefit from an increased MMSE score, and administering 1000 mg of an anti-Gal3 antibody, wherein the antibody is administered once weekly for 5 weeks, is disclosed.

In some embodiments, a method of increasing a subject's Mini Mental State Examination (MMSE) score, the method comprising: identifying a subject as likely to benefit from an increased MMSE score, and administering 140 mg of an anti-Gal3 antibody once weekly for 5 weeks followed by administration of 4000 mg of the antibody once monthly for one or more months, is disclosed.

In some embodiments, a method of increasing a subject's Mini Mental State Examination (MMSE) score, the method comprising: identifying a subject as likely to benefit from an increased MMSE score, and administering 420 mg of an anti-Gal3 antibody once weekly for 5 weeks followed by administration of 4000 mg of the antibody once monthly for one or more months, is disclosed.

In some embodiments, a method of increasing a subject's Mini Mental State Examination (MMSE) score, the method comprising: identifying a subject as likely to benefit from an increased MMSE score, and administering 1000 mg of an anti-Gal3 antibody once weekly for 5 weeks followed by administration of 4000 mg of the antibody once monthly for one or more months, is disclosed.

In some embodiments, a method of increasing a subject's Mini Mental State Examination (MMSE) score, the method comprising: identifying a subject as likely to benefit from an increased MMSE score, and administering 1000 mg of an anti-Gal3 antibody once weekly for 5 weeks followed by administration of 4000 mg of the antibody once monthly for one or more months, and wherein administration of the antibody increases the subject's MMSE score by up to about 11 points, is disclosed.

In some embodiments, a method of increasing a subject's EQ-5D-5L score, the method comprising: identifying a subject as likely to benefit from an increased EQ-5D-5L score, and administering a therapeutically effective amount of an antibody, wherein the antibody binds to Gal3, is disclosed.

In some embodiments, a method of increasing a subject's a subject's EuroQol 5-Dimension 5-Level (EQ-5D-5L) score, the method comprising: identifying a subject as having Alzheimer's disease (AD), and administering a therapeutically effective amount of an antibody, wherein the antibody binds to Gal3, is disclosed.

In some embodiments, a method of increasing a subject's a subject's EuroQol 5-Dimension 5-Level (EQ-5D-5L) score, the method comprising: identifying a subject as having dementia, and administering a therapeutically effective amount of an antibody, wherein the antibody binds to Gal3, is disclosed.

In some embodiments, a method of decreasing a subject's Neuropsychiatric Inventory (NPI) score, the method comprising: identifying a subject as likely to benefit from a decreased Neuropsychiatric Inventory (NPI) score, and administering a therapeutically effective amount of an antibody, wherein the antibody binds to Gal3, is disclosed.

In some embodiments, a method of decreasing a subject's a subject's Neuropsychiatric Inventory (NPI) score, the method comprising: identifying a subject as having Alzheimer's disease (AD), and administering a therapeutically effective amount of an antibody, wherein the antibody binds to Gal3, is disclosed.

In some embodiments, a method of decreasing a subject's a subject's Neuropsychiatric Inventory (NPI) score, the method comprising: identifying a subject as having dementia, and administering a therapeutically effective amount of an antibody, wherein the antibody binds to Gal3, is disclosed.

In some embodiments, a method of decreasing a subject's Clinical Dementia Rating Scale-Sum of Boxes (CDR-SB), the method comprising: identifying a subject as likely to benefit from a decreased CDR-SB score, and administering a therapeutically effective amount of an antibody, wherein the antibody binds to Gal3, is disclosed.

In some embodiments, a method of decreasing a subject's Clinical Dementia Rating Scale-Sum of Boxes (CDR-SB) score, the method comprising: identifying a subject as having Alzheimer's disease (AD), and Administering an antibody, wherein the antibody binds to Gal3, is disclosed.

In some embodiments, a method of decreasing a subject's Clinical Dementia Rating Scale-Sum of Boxes (CDR-SB) score, the method comprising: identifying a subject as having dementia, and administering an antibody, wherein the antibody binds to Gal3, is disclosed.

In some embodiments, a method of decreasing a subject's Clinical Dementia Rating Scale-Sum of Boxes (CDR-SB) score, the method comprising: identifying a subject as likely to benefit from a decreased CDR-SB score, and administering 140 mg of an anti-Gal3 antibody, wherein the antibody is administered once weekly for 5 weeks, is disclosed.

In some embodiments, a method of decreasing a subject's Clinical Dementia Rating Scale-Sum of Boxes (CDR-SB) score, the method comprising: identifying a subject as likely to benefit from a decreased CDR-SB score, and administering 420 mg of an anti-Gal3 antibody, wherein the antibody is administered once weekly for 5 weeks, is disclosed.

In some embodiments, a method of decreasing a subject's Clinical Dementia Rating Scale-Sum of Boxes (CDR-SB) score, the method comprising: identifying a subject as likely to benefit from a decreased CDR-SB score, and administering 1000 mg of an anti-Gal3 antibody, wherein the antibody is administered once weekly for 5 weeks, is disclosed.

In some embodiments, a method of decreasing a subject's Clinical Dementia Rating Scale-Sum of Boxes (CDR-SB) score, the method comprising: identifying a subject as likely to benefit from a decreased CDR-SB score, and administering an anti-Gal3 antibody, wherein 140 mg of the antibody is administered once weekly for 5 weeks followed by administration of 4000 mg of the antibody once monthly for one or more months, is disclosed.

In some embodiments, a method of decreasing a subject's Clinical Dementia Rating Scale-Sum of Boxes (CDR-SB) score, the method comprising: identifying a subject as likely to benefit from a decreased CDR-SB score, and administering an anti-Gal3 antibody, wherein 420 mg of the antibody is administered once weekly for 5 weeks followed by administration of 4000 mg of the antibody once monthly for one or more months, is disclosed.

In some embodiments, a method of decreasing a subject's Clinical Dementia Rating Scale-Sum of Boxes (CDR-SB) score, the method comprising: identifying a subject as likely to benefit from a decreased CDR-SB score, and administering an anti-Gal3 antibody, wherein 1000 mg of the antibody is administered once weekly for 5 weeks followed by administration of 4000 mg of the antibody once monthly for one or more months, is disclosed.

In some embodiments, a method of decreasing a subject's Clinical Dementia Rating Scale-Sum of Boxes (CDR-SB) score, the method comprising: identifying a subject as likely to benefit from a decreased CDR-SB score, and administering an anti-Gal3 antibody, wherein 1000 mg of the antibody is administered once weekly for 5 weeks followed by administration of 4000 mg of the antibody once monthly for one or more months, and wherein administration of the antibody decreases the subject's Clinical Dementia Rating Scale-Sum of Boxes (CDR-SB) score by up to about 0.5 points, is disclosed.

In some embodiments, a method of increasing a subject's Cognitive Drug Research battery score, the method comprising: identifying a subject as likely to benefit from an increased Cognitive Drug Research battery score, and administering a therapeutically effective amount of an antibody, wherein the antibody binds to Gal3, is disclosed.

In some embodiments, a method of increasing a subject's Cognitive Drug Research battery score, the method comprising: identifying a subject as having Alzheimer's disease (AD), and administering a therapeutically effective amount of an antibody, wherein the antibody binds to Gal3, is disclosed.

In some embodiments, a method of increasing a subject's Cognitive Drug Research battery score, the method comprising: identifying a subject as having dementia, and administering a therapeutically effective amount of an antibody, wherein the antibody binds to Gal3, is disclosed.

In some embodiments, a method of increasing a subject's strength, locomotor functions, and/or balance, the method comprising: identifying a subject as likely to benefit from increased strength, locomotor functions, and/or balance, and administering a therapeutically effective amount of an antibody, wherein the antibody binds Gal3, is disclosed.

In some embodiments, a method of increasing a subject's strength, locomotor functions, and/or balance, the method comprising: identifying a subject as having Alzheimer's disease (AD), and administering a therapeutically effective amount of an antibody, wherein the antibody binds Gal3, is disclosed.

In some embodiments, a method of increasing a subject's strength, locomotor functions, and/or balance, the method comprising: identifying a subject as having dementia, and administering a therapeutically effective amount of an antibody, wherein the antibody binds Gal3, is disclosed.

In some embodiments, a method of decreasing the frequency and/or severity of symptoms related to aging and/or aging related senescence in a subject in need thereof, the method comprising: identifying a subject as likely to benefit from decreased frequency and/or severity of symptoms related to aging and/or aging related senescence, and administering a therapeutically effective amount of an antibody, wherein the antibody binds Gal3, is disclosed.

In some embodiments, a method of increasing a subject's whole brain volume, the method comprising: identifying a subject as likely to benefit from an increased whole brain volume, and administering a therapeutically effective amount of an antibody, wherein the antibody binds to Gal3, is disclosed.

In some embodiments, a method of decreasing brain atrophy in a subject, the method comprising: identifying a subject as likely to benefit from decreased brain atrophy, and administering a therapeutically effective amount of an antibody, wherein the antibody binds to Gal3, is disclosed.

In some embodiments, a method of increasing a subject's performance on one or more standard cognitive assessments, the method comprising: identifying a subject as likely to benefit from increased performance on the one or more standard cognitive assessments, and administering a therapeutically effective amount of an antibody, wherein the antibody binds to Gal3, is disclosed.

In some embodiments, a method of treating a patient, the method comprising: identifying a patient, wherein the patient is identified as having a risk of a decreased MMSE score, decreased EQ-5D-5L score, increased CDR-SB score, decreased NPI score, decreased Cognitive Drug Research battery, increased brain atrophy, decreased whole brain volume, increased symptoms related to aging and/or aging related senescence, decreased cognitive ability, decreased strength, decreased locomotor functions, decreased balance, or any combination thereof, and administering a therapeutically effective amount of an anti Gal3 Ab to the patient to reduce said risk, is disclosed.

In some embodiments, a method of decreasing a subject's Clinical Dementia Rating Scale-Sum of Boxes (CDR-SB), the method comprising: identifying a subject as likely to benefit from a decreased CDR-SB score, and administering a therapeutically effective amount of an antibody, wherein the antibody binds to Gal3, and wherein the anti-Gal3 antibody is administered 5 times in a month via intravenous injection over the course of 1-hour for a total administered dose of 140-4000 mg, followed by administration once monthly, every 28 days, via intravenous injection over the course of 1-hour for a total administered dose of 140-4000 mg, is disclosed.

In some embodiments, a method of increasing a subject's Mini Mental State Examination (MMSE) score, the method comprising: identifying a subject as likely to benefit from an increased MMSE score, and administering a therapeutically effective amount of an antibody, wherein the antibody binds to Gal3, and wherein the anti-Gal3 antibody is administered 5 times in a month via intravenous injection over the course of 1-hour for a total administered dose of 140-4000 mg, followed by administration once monthly, every 28 days, via intravenous injection over the course of 1-hour for a total administered dose of 140-4000 mg, is disclosed.

In some embodiments, a method of decreasing a subject's Clinical Dementia Rating Scale-Sum of Boxes (CDR-SB), the method comprising: identifying a subject as likely to benefit from a decreased CDR-SB score, and administering a therapeutically effective amount of TB006 antibody, wherein the TB006 antibody is administered 5 times in a month via intravenous injection over the course of 1-hour for a total administered dose of 140-4000 mg, followed by administration once monthly, every 28 days, via intravenous injection over the course of 1-hour for a total administered dose of 140-4000 mg, is disclosed.

In some embodiments, a method of increasing a subject's Mini Mental State Examination (MMSE) score, the method comprising: identifying a subject as likely to benefit from an increased MMSE score, and administering a therapeutically effective amount of TB006 antibody, wherein the TB006 antibody is administered 5 times in a month via intravenous injection over the course of 1-hour for a total administered dose of 140-4000 mg, followed by administration once monthly, every 28 days, via intravenous injection over the course of 1-hour for a total administered dose of 140-4000 mg, is disclosed.

In some embodiments, a method of decreasing a subject's Clinical Dementia Rating Scale-Sum of Boxes (CDR-SB), the method comprising: identifying a subject as likely to benefit from a decreased CDR-SB score, and administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein the therapeutic formulation is administered 5 times in a month via intravenous injection over the course of 1-hour for a total administered dose of 140-4000 mg, followed by administration once monthly, every 28 days, via intravenous injection over the course of 1-hour for a total administered dose of 140-4000 mg, is disclosed.

In some embodiments, a method of increasing a subject's Mini Mental State Examination (MMSE) score, the method comprising: identifying a subject as likely to benefit from an increased MMSE score, and administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein the therapeutic formulation is administered 5 times in a month via intravenous injection over the course of 1-hour for a total administered dose of 140-4000 mg, followed by administration once monthly, every 28 days, via intravenous injection over the course of 1-hour for a total administered dose of 140-4000 mg, is disclosed.

In some embodiments, a method of decreasing a subject's Clinical Dementia Rating Scale-Sum of Boxes (CDR-SB), the method comprising: identifying a subject as likely to benefit from a decreased CDR-SB score, and administering a therapeutically effective amount of an antibody, wherein the antibody binds to Gal3, and wherein the anti-Gal3 antibody is administered 5 times in a month via intravenous injection over the course of 1-hour for a total administered dose of 140 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg, is disclosed.

In some embodiments, a method of increasing a subject's Mini Mental State Examination (MMSE) score, the method comprising: identifying a subject as likely to benefit from an increased MMSE score, and administering a therapeutically effective amount of an antibody, wherein the antibody binds to Gal3, and wherein the anti-Gal3 antibody is administered 5 times in a month via intravenous injection over the course of 1-hour for a total administered dose of 140 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg, is disclosed.

In some embodiments, a method of decreasing a subject's Clinical Dementia Rating Scale-Sum of Boxes (CDR-SB), the method comprising: identifying a subject as likely to benefit from a decreased CDR-SB score, and administering a therapeutically effective amount of TB006 antibody, wherein the TB006 antibody is administered 5 times in a month via intravenous injection over the course of 1-hour for a total administered dose of 140 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg, is disclosed.

In some embodiments, a method of increasing a subject's Mini Mental State Examination (MMSE) score, the method comprising: identifying a subject as likely to benefit from an increased MMSE score, and administering a therapeutically effective amount of TB006 antibody, wherein the TB006 antibody is administered 5 times in a month via intravenous injection over the course of 1-hour for a total administered dose of 140 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg, is disclosed.

In some embodiments, a method of decreasing a subject's Clinical Dementia Rating Scale-Sum of Boxes (CDR-SB), the method comprising: identifying a subject as likely to benefit from a decreased CDR-SB score, and administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein the therapeutic formulation is administered 5 times in a month via intravenous injection over the course of 1-hour for a total administered dose of 140 mg, followed by administration 5 times in a month for a total administered dose of 1000 mg, followed by administration once monthly, every 28 days, via intravenous injection over the course of 1-hour for a total administered dose of 4000 mg, is disclosed.

In some embodiments, a method of increasing a subject's Mini Mental State Examination (MMSE) score, the method comprising: identifying a subject as likely to benefit from an increased MMSE score, and administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein the therapeutic formulation is administered 5 times in a month via intravenous injection over the course of 1-hour for a total administered dose of 140 mg, followed by administration 5 times in a month for a total administered dose of 1000 mg, followed by administration once monthly, every 28 days, via intravenous injection over the course of 1-hour for a total administered dose of 4000 mg, is disclosed.

In some embodiments, a method of decreasing a subject's Clinical Dementia Rating Scale-Sum of Boxes (CDR-SB), the method comprising: identifying a subject as likely to benefit from a decreased CDR-SB score, and administering a therapeutically effective amount of an antibody, wherein the antibody binds to Gal3, and wherein the anti-Gal3 antibody is administered 5 times in a month via intravenous injection over the course of 1-hour for a total administered dose of 420 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg, is disclosed.

In some embodiments, a method of increasing a subject's Mini Mental State Examination (MMSE) score, the method comprising: identifying a subject as likely to benefit from an increased MMSE score, and administering a therapeutically effective amount of an antibody, wherein the antibody binds to Gal3, and wherein the anti-Gal3 antibody is administered 5 times in a month via intravenous injection over the course of 1-hour for a total administered dose of 420 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg, is disclosed.

In some embodiments, a method of decreasing a subject's Clinical Dementia Rating Scale-Sum of Boxes (CDR-SB), the method comprising: identifying a subject as likely to benefit from a decreased CDR-SB score, and administering a therapeutically effective amount of TB006 antibody, wherein the TB006 antibody is administered 5 times in a month via intravenous injection over the course of 1-hour for a total administered dose of 420 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg, is disclosed.

In some embodiments, a method of increasing a subject's Mini Mental State Examination (MMSE) score, the method comprising: identifying a subject as likely to benefit from an increased MMSE score, and administering a therapeutically effective amount of TB006 antibody, wherein the TB006 antibody is administered 5 times in a month via intravenous injection over the course of 1-hour for a total administered dose of 420 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg, is disclosed.

In some embodiments, a method of decreasing a subject's Clinical Dementia Rating Scale-Sum of Boxes (CDR-SB), the method comprising: identifying a subject as likely to benefit from a decreased CDR-SB score, and administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein the therapeutic formulation is administered 5 times in a month via intravenous injection over the course of 1-hour for a total administered dose of 420 mg, followed by administration 5 times in a month for a total administered dose of 1000 mg, followed by administration once monthly, every 28 days, via intravenous injection over the course of 1-hour for a total administered dose of 4000 mg, is disclosed.

In some embodiments, a method of increasing a subject's Mini Mental State Examination (MMSE) score, the method comprising: identifying a subject as likely to benefit from an increased MMSE score, and administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein the therapeutic formulation is administered 5 times in a month via intravenous injection over the course of 1-hour for a total administered dose of 420 mg, followed by administration 5 times in a month for a total administered dose of 1000 mg, followed by administration once monthly, every 28 days, via intravenous injection over the course of 1-hour for a total administered dose of 4000 mg, is disclosed.

In some embodiments, a method of decreasing a subject's Clinical Dementia Rating Scale-Sum of Boxes (CDR-SB), the method comprising: identifying a subject as likely to benefit from a decreased CDR-SB score, and administering a therapeutically effective amount of an antibody, wherein the antibody binds to Gal3, and wherein the anti-Gal3 antibody is administered 5 times in a month via intravenous injection over the course of 1-hour for a total administered dose of 1000 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg, is disclosed.

In some embodiments, a method of increasing a subject's Mini Mental State Examination (MMSE) score, the method comprising: identifying a subject as likely to benefit from an increased MMSE score, and administering a therapeutically effective amount of an antibody, wherein the antibody binds to Gal3, and wherein the anti-Gal3 antibody is administered 5 times in a month via intravenous injection over the course of 1-hour for a total administered dose of 14000 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg, is disclosed.

In some embodiments, a method of decreasing a subject's Clinical Dementia Rating Scale-Sum of Boxes (CDR-SB), the method comprising: identifying a subject as likely to benefit from a decreased CDR-SB score, and administering a therapeutically effective amount of TB006 antibody, wherein the TB006 antibody is administered 5 times in a month via intravenous injection over the course of 1-hour for a total administered dose of 1000 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg, is disclosed.

In some embodiments, a method of increasing a subject's Mini Mental State Examination (MMSE) score, the method comprising: identifying a subject as likely to benefit from an increased MMSE score, and administering a therapeutically effective amount of TB006 antibody, wherein the TB006 antibody is administered 5 times in a month via intravenous injection over the course of 1-hour for a total administered dose of 1000 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg, is disclosed.

In some embodiments, a method of decreasing a subject's Clinical Dementia Rating Scale-Sum of Boxes (CDR-SB), the method comprising: identifying a subject as likely to benefit from a decreased CDR-SB score, and administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein the therapeutic formulation is administered 5 times in a month via intravenous injection over the course of 1-hour for a total administered dose of 1000 mg, followed by administration 5 times in a month for a total administered dose of 1000 mg, followed by administration once monthly, every 28 days, via intravenous injection over the course of 1-hour for a total administered dose of 4000 mg, is disclosed.

In some embodiments, a method of increasing a subject's Mini Mental State Examination (MMSE) score, the method comprising: identifying a subject as likely to benefit from an increased MMSE score, and administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein the therapeutic formulation is administered 5 times in a month via intravenous injection over the course of 1-hour for a total administered dose of 1000 mg, followed by administration 5 times in a month for a total administered dose of 1000 mg, followed by administration once monthly, every 28 days, via intravenous injection over the course of 1-hour for a total administered dose of 4000 mg, is disclosed.

In some embodiments, a method of diagnosing a subject as having, or likely having one or more diseases, the method comprising: administering an anti-Gal3 antibody or binding fragment thereof to a subject; wherein the anti-Gal3 antibody or binding fragment thereof comprises a detectable payload; and detecting the payload; wherein detection of the payload indicates that the subject has or likely has one or more diseases, is disclosed.

In some embodiments, a method of diagnosing a subject as having, or likely having a neurological disorder, the method comprising: administering an anti-Gal3 antibody or binding fragment thereof to a subject; wherein the anti-Gal3 antibody or binding fragment thereof comprises a detectable payload; and detecting the payload; wherein detection of the payload indicates that the subject has or likely has a neurological disorder, is disclosed.

In some embodiments, a method of diagnosing a subject as having, or likely having a proteopathy, the method comprising: administering an anti-Gal3 antibody or binding fragment thereof to a subject; wherein the anti-Gal3 antibody or binding fragment thereof comprises a detectable payload; and detecting the payload; wherein detection of the payload indicates that the subject has or likely has a proteopathy, is disclosed.

In some embodiments, a method of identifying a subject as having, or likely having Alzheimer's disease (AD), the method comprising, administering an anti-Gal3 antibody or binding fragment thereof to a subject; wherein the anti-Gal3 antibody or binding fragment thereof comprises a detectable payload; and detecting the payload; wherein detection of the payload in the subject's brain indicates that the subject has or likely has AD, is disclosed.

In some embodiments, a method of identifying the severity of Alzheimer's disease (AD) in a subject as having AD, the method comprising: administering an anti-Gal3 antibody or binding fragment thereof to a subject; wherein the anti-Gal3 antibody or binding fragment thereof comprises a detectable payload; and detecting the payload; wherein the level of payload detection in the subject's brain indicates the severity of the subject's AD, is disclosed.

In some embodiments, an anti-Gal3 antibody or binding fragment thereof for use in diagnosing a subject with Alzheimer's disease (AD) is disclosed. In some embodiments, the anti-Gal3 antibody or binding fragment thereof binds to one or more peptides of SEQ ID NOs: 3-26; wherein the anti-Gal3 antibody or binding fragment thereof comprises a detectable payload; and wherein detection of the anti-Gal3 antibody in the subject's brain is indicates that the subject has or likely has AD.

### BRIEF DESCRIPTION OF THE DRAWINGS

FIG. 1 is a flow chart depicting some embodiments of a method of increasing a subject's Mini Mental State Ex69amination (MMSE) score.

FIG. 2 is a flow chart depicting some embodiments of a method of increasing a subject's Mini Mental State Examination (MMSE) score.

FIG. 3 is a flow chart depicting some embodiments of a method of increasing a subject's a subject's EQ-5D-5L score.

FIG. 4 is a flow chart depicting some embodiments of a method of increasing a subject's EQ-5D-5L score.

FIG. 5 is a flow chart depicting some embodiments of a method of decreasing a subject's Neuropsychiatric Inventory (NPI) score.

FIG. 6 is a flow chart depicting some embodiments of a method of decreasing a subject's Neuropsychiatric Inventory (NPI) score.

FIG. 7 is a flow chart depicting some embodiments of a method of increasing a subject's Clinical Dementia Rating Scale-Sum of Boxes (CDR-SB).

FIG. 8 is a flow chart depicting some embodiments of a method of increasing a subject's Clinical Dementia Rating Scale-Sum of Boxes (CDR-SB) score.

FIG. 9 is a flow chart depicting some embodiments of a method of increasing a subject's Cognitive Drug Research battery score.

FIG. 10 is a flow chart depicting some embodiments of a method of increasing a subject's Cognitive Drug Research battery score.

FIG. 11 is a flow chart depicting some embodiments of a method of increasing a subject's strength, locomotor functions, and/or balance.

FIG. 12 is a flow chart depicting some embodiments of a method of increasing a subject's strength, locomotor functions, and/or balance.

FIG. 13 is a flow chart depicting some embodiments of a method of decreasing the frequency and/or severity of symptoms related to aging and/or aging related senescence in a subject in need thereof.

FIG. 14 is a flow chart depicting some embodiments of a method of increasing a subject's whole brain volume.

FIG. 15 is a flow chart depicting some embodiments of a method of decreasing brain atrophy in a subject.

FIG. 16 is a flow chart depicting some embodiments of a method of increasing a subject's performance on one or more standard cognitive assessments.

FIG. 17 is a flow chart depicting some embodiments of a method of treating a patient.

FIG. 18A-D is a flow chart depicting some embodiments of a method for testing the safety and efficacy of an anti-Gal3 antibody. FIG. 18A depicts some embodiments of a method for testing the safety and efficacy of an anti-Gal3 antibody. FIG. 18B is a flow chart depicting embodiments of a dosing and testing schedule. FIG. 18C depicts some embodiments of a Clinical Dementia Rating-Sum of Boxes (CDR-SB) score keeping template. FIG. 18D depicts some embodiments of a Mini-Mental State Examination (MMSE) score keeping template.

FIG. 19 depicts graphs of some embodiments of a subject's CDR-SB score following administration of an anti-Gal3 antibody at different doses.

FIG. 20 depicts graphs of some embodiments of a subject's MMSE score following administration of an anti-Gal3 antibody at different doses.

FIGS. 21A-21B depicts embodiments of data from a cohort of subjects treated with an anti-Gal3 antibody. FIG. 21A is a heat map depicting some embodiments of the magnitude of change in a subject's CDR-SB score following administration of an anti-Gal3 antibody. FIG. 21B are graphs depicting embodiments of subject's CDR-SB score following administration of an anti-Gal3 antibody.

FIGS. 22A-22B depicts embodiments of data from a cohort of subjects treated with an anti-Gal3 antibody. FIG. 22A is a heat map depicting some embodiments of the magnitude of change in a subject's MMSE score following administration of an anti-Gal3 antibody. FIG. 22B are graphs depicting embodiments of subject's MMSE score following administration of an anti-Gal3 antibody.

FIG. 23 is a flow chart depicting embodiments of a dosing and testing schedule for biomarker analysis.

FIG. 24A is flow charts depicting some embodiments of a method for testing the safety and efficacy of an anti-Gal3 antibody. FIG. 24B is graphs depicting some embodiments of HbA1c changes in AD patients following anti-Gal3 antibody administration.

FIG. 25 is a table depicting some embodiments of the adverse events and their frequencies in a cohort of subject's treated with anti-Gal3 antibody.

FIG. 26 is a graph showing some embodiments of a pharmacokinetic analysis of an anti-Gal3 antibody.

FIG. 27 is a table depicting embodiments of study interventions administered to subjects.

FIG. 28 is a table depicting some embodiments for packaging and labeling of an anti-Gal3 antibody.

FIG. 29 is a table depicting some embodiments of a Schedule of Activities (SoA) according to visit number.

FIG. 30 is a table depicting embodiments of blood volumes required for various assessments.

FIG. 31 is a flow chart depicting some embodiments of a method for assessing the safety and efficacy of anti-Gal3 antibodies.

FIG. 32 is a table depicting some embodiments of organizing patients for analysis.

FIG. 33 is a table depicting some embodiments of laboratory test that may be performed on subjects treated with anti-Gal3 antibodies.

FIG. 34 is a table depicting some embodiments of liver chemistry stopping events.

FIG. 35 depicts protein sequences of Gal3 and exemplary proteins that exhibit pathogenic aggregation.

FIG. 36 depicts peptide sequences of Gal3 used to generate and analyze antibodies.

FIG. 37A depicts exemplary variable heavy chain complementarity-determining region (CDR) 1 for anti-Gal3 antibodies disclosed herein. In some embodiments, any of the compositions or methods provided herein can include one or more of the variable heavy chain CDR1 provided herein.

FIG. 37B depicts exemplary variable heavy chain CDR2 for anti-Gal3 antibodies disclosed herein. In some embodiments, any of the compositions or methods provided herein can include one or more of the variable heavy chain CDR2 provided herein.

FIG. 37C depicts exemplary variable heavy chain CDR3 for anti-Gal3 antibodies disclosed herein. In some embodiments, any of the compositions or methods provided herein can include one or more of the variable heavy chain CDR3 provided herein.

FIG. 38A depicts exemplary variable light chain CDR1 for anti-Gal3 antibodies disclosed herein. In some embodiments, any of the compositions or methods provided herein can include one or more of the variable light chain CDR1 provided herein.

FIG. 38B depicts exemplary variable light chain CDR2 for anti-Gal3 antibodies disclosed herein. In some embodiments, any of the compositions or methods provided herein can include one or more of the variable light chain CDR2 provided herein.

FIG. 38C depicts exemplary variable light chain CDR3 for anti-Gal3 antibodies disclosed herein. In some embodiments, any of the compositions or methods provided herein can include one or more of the variable light chain CDR3 provided herein.

FIG. 39 depicts exemplary heavy chain variable region (VH) sequences for anti-Gal3 antibodies disclosed herein. In some embodiments, any of the compositions or methods provided herein can include one or more of the VH sequences provided herein.

FIG. 40 depicts exemplary light chain variable region (VL) sequences for anti-Gal3 antibodies disclosed herein. In some embodiments, any of the compositions or methods provided herein can include one or more of the VL sequences provided herein.

FIG. 41 depicts exemplary combinations of heavy and light chain CDRs (CDR1, CDR2, and CDR3) of exemplary anti-Gal3 antibodies disclosed herein. In some embodiments, any of the compositions or methods provided herein can include one or more of the heavy and light chain CDR combinations provided herein.

FIG. 42 depicts exemplary combinations of heavy and light chain variable regions of exemplary anti-Gal3 antibodies disclosed herein. In some embodiments, any of the compositions or methods provided herein can include one or more of the heavy and light chain variable region combinations provided herein.

FIG. 43 depicts exemplary heavy chain (HC) sequences and light chain (LC) sequences, and possible pairings for exemplary anti-Gal3 antibodies disclosed herein. In some embodiments, any of the compositions or methods provided herein can include one or more of the HC or LC, or pairs of HC and LC sequences provided herein.

FIG. 44 depicts antibody names used throughout the present disclosure refer to the same antibody (with exemplary peptide and nucleic acid sequences provided elsewhere in the disclosure and appropriately attributed to at least one of the depicted names) and may be used interchangeably. The names shown in a column correspond to the same antibody.

FIG. 45 depicts an alignment of hinge and constant heavy chain domain 2 (CH2) domain amino acid sequences of wild-type human immunoglobulin G1 (IgG1), IgG2 and IgG4 as well as their sigma variants. The alignment above uses EU numbering. Residues identical to wild-type IgG1 are indicated as dots; gaps are indicated with hyphens. Sequence is given explicitly if it differs from wild-type IgG1 or from the parental subtype for σ variants. Open boxes beneath the alignment correspond to International Immunogenetics Information System (IMGT) strand definitions. Boxes beneath the alignment correspond to the strand and helix secondary structure assignment for wild-type IgG1. Residues 267-273 form the BC loop and 322-332 form the FG loop. Also provided are exemplary constant regions for human IgG4 heavy (S228P mutant) and light (kappa) chains (SEQ ID NOs: 931-932) and murine IgG2A (LALAPG and LALA mutants) (SEQ ID NOs: 933-934). In some embodiments, any one or more of the VH/VL and/or CDRs provided in the other figures or otherwise disclosed herein can be paired with any one or more of the exemplary constant regions provided herein.

FIG. 46 depicts nucleic acid sequences that encode for exemplary heavy chain variable regions of anti-Gal3 antibodies disclosed herein. In some embodiments, any of the compositions or methods provided herein can include one or more of the heavy chain variable regions encoded by the nucleic acids provided herein.

FIG. 47 depicts nucleic acid sequences that encode for exemplary light chain variable regions of anti-Gal3 antibodies disclosed herein. In some embodiments, any of the compositions or methods provided herein can include one or more of the light chain variable regions encoded by the nucleic acids provided herein.

FIG. 48 depicts nucleic acid sequences that encode for exemplary heavy chains of anti-Gal3 antibodies disclosed herein. In some embodiments, any of the compositions or methods provided herein can include one or more of the heavy chains encoded by the nucleic acids provided herein.

FIG. 49 depicts nucleic acid sequences that encode for exemplary light chains of anti-Gal3 antibodies disclosed herein. In some embodiments, any of the compositions or methods provided herein can include one or more of the light chains encoded by the nucleic acids provided herein.

FIG. 50A-B depicts an exemplary alignment for the heavy chain CDRs (FIG. 50A) and light chain CDRs (FIG. 50B) for the exemplary anti-Gal3 antibodies disclosed herein.

FIG. 51 is a flow chart depicting some embodiments of a method of increasing a subject's MMSE score.

FIG. 52 is a flow chart depicting some embodiments of a method of increasing a subject's EuroQol 5-Dimension 5-Level (EQ-5D-5L).

FIG. 53 is a flow chart depicting some embodiments of a method of decreasing a subject's NPI.

FIG. 54 is a flow chart depicting some embodiments of a method of decreasing a subject's CDR-SB score.

FIG. 55 is a flow chart depicting some embodiments of a method of increasing a subject's Cognitive Drug Research battery score.

FIG. 56 is a flow chart depicting some embodiments of a method of increasing a subject's strength, locomotor functions, and/or balance.

FIGS. 57A-57B are tables depicting some embodiments of endpoints in a study of the safety efficacy of anti-Gal3 antibodies. FIG. 57A is a table depicting some embodiments of endpoints in part one of a two part study of the safety efficacy of anti-Gal3 antibodies. FIG. 57B is a table depicting some embodiments of endpoints in part two of a two part study of the safety efficacy of anti-Gal3 antibodies.

FIG. 58 is a chart depicting some embodiments of study design and treatment schema for parts 1 and 2 of a two part study of the safety efficacy of anti-Gal3 antibodies.

FIG. 59 is a chart depicting some embodiments of study design and treatment schema interleaving parts 1 and 2 of a two part study of the safety efficacy of anti-Gal3 antibodies.

FIGS. 60A-60B depict some embodiments of a schedule of activities (SoA) in a two part study of the safety efficacy of anti-Gal3 antibodies. FIG. 60A is a table depicting some embodiments of a SoA in part one of a two part study of the safety efficacy of anti-Gal3 antibodies. FIG. 60B is a table depicting some embodiments of a SoA in part two of a two part study of the safety efficacy of anti-Gal3 antibodies.

FIG. 61 is a table depicting some embodiments of study interventions administered in a study of the safety and efficacy of anti-Gal3 antibodies.

FIG. 62A-B depicts some embodiments of blood samples and volumes drawn from subject's in a two part study of the safety efficacy of anti-Gal3 antibodies. FIG. 62A is a table depicting some embodiments of blood samples and volumes drawn from subject's in part one of a two part study of the safety efficacy of anti-Gal3 antibodies. FIG. 62B is a table depicting some embodiments of blood samples and volumes drawn from subject's in part two of a two part study of the safety efficacy of anti-Gal3 antibodies.

FIG. 63 is a table depicting some embodiments of protocol-required clinical laboratory safety assessments.

FIG. 64 is a chart depicting some embodiments of study design, treatment schema, and endpoints for parts 1 and 2 of a two part study of the safety efficacy of anti-Gal3 antibodies.

FIG. 65 is a line graph depicting some embodiments of the effect of TB006 on the global CDR-SB scores in patients undergoing a phase 1b/2a, multi-dose, multi-center study to assess the safety, tolerability, pharmacokinetics, pharmacodynamics, and efficacy of TB006 in patients with Alzheimer's Disease.

FIG. 66 is a line graph depicting some embodiments of the effect of TB006 on Memory CDR-SB subdomain scores in patients undergoing a phase 1b/2a, multi-dose, multi-center study to assess the safety, tolerability, pharmacokinetics, pharmacodynamics, and efficacy of TB006 in patients with Alzheimer's Disease.

FIG. 67 is a line graph depicting some embodiments of the effect of TB006 on the Orientation CDR-SB subdomain scores in patients undergoing a phase 1b/2a, multi-dose, multi-center study to assess the safety, tolerability, pharmacokinetics, pharmacodynamics, and efficacy of TB006 in patients with Alzheimer's Disease.

FIG. 68 is a line graph depicting some embodiments of the effect of TB006 on the Judgment and Problem Solving CDR-SB subdomain scores in patients undergoing a phase 1b/2a, multi-dose, multi-center study to assess the safety, tolerability, pharmacokinetics, pharmacodynamics, and efficacy of TB006 in patients with Alzheimer's Disease.

FIG. 69 is a line graph depicting some embodiments of the effect of TB006 on the Community Affairs CDR-SB subdomain scores in patients undergoing a phase 1b/2a, multi-dose, multi-center study to assess the safety, tolerability, pharmacokinetics, pharmacodynamics, and efficacy of TB006 in patients with Alzheimer's Disease.

FIG. 70 is a line graph depicting some embodiments of the effect of TB006 on the Home and Hobbies CDR-SB subdomain scores in patients undergoing a phase 1b/2a, multi-dose, multi-center study to assess the safety, tolerability, pharmacokinetics, pharmacodynamics, and efficacy of TB006 in patients with Alzheimer's Disease.

FIG. 71 is a line graph depicting some embodiments of the effect of TB006 on the Personal Care CDR-SB subdomain scores in patients undergoing a phase 1b/2a, multi-dose, multi-center study to assess the safety, tolerability, pharmacokinetics, pharmacodynamics, and efficacy of TB006 in patients with Alzheimer's Disease.

FIG. 72 is a line graph depicting some embodiments of the effect of TB006 on CDR-SB scores in patients undergoing a phase 1b/2a, multi-dose, multi-center study to assess the safety, tolerability, pharmacokinetics, pharmacodynamics, and efficacy of TB006 in patients with Alzheimer's Disease.

FIG. 73 is a line graph depicting some embodiments of the effect of TB006 on Aβ plasma levels in patients undergoing a phase 1b/2a, multi-dose, multi-center study to assess the safety, tolerability, pharmacokinetics, pharmacodynamics, and efficacy of TB006 in patients with Alzheimer's Disease.

FIG. 74 is a line graph depicting some embodiments of the effect of TB006 on hippocampal volume in patients undergoing a phase 1b/2a, multi-dose, multi-center study to assess the safety, tolerability, pharmacokinetics, pharmacodynamics, and efficacy of TB006 in patients with Alzheimer's Disease.

FIG. 75 is a line graph depicting some embodiments of the effect of TB006 on intracranial volume in patients undergoing a phase 1b/2a, multi-dose, multi-center study to assess the safety, tolerability, pharmacokinetics, pharmacodynamics, and efficacy of TB006 in patients with Alzheimer's Disease.

FIG. 76 depicts some embodiments of PET scans of subjects at different timepoints prior to and following treatment with TB006.

FIG. 77 is an illustrative representation of some embodiments of the mechanism of action of anti-Gal3 antibodies (TB006).

FIG. 78 depicts some exemplary embodiments of TB006 amino acid sequences. Any of the methods provided herein can employ one or more components of the antibody shown in FIG. 78, including the heavy and light chains, the heavy and light variable regions, 1, 2, 3, 4, 5, or 6 of the CDRS therein.

FIG. 79 depicts some embodiments of PET scans of a subject at different timepoints following treatment with TB006.

FIG. 80 is a line graph depicting some embodiments of the effect of TB006 on left lateral ventricle volume in patients undergoing a phase 1b/2a, multi-dose, multi-center study to assess the safety, tolerability, pharmacokinetics, pharmacodynamics, and efficacy of TB006 in patients with Alzheimer's Disease.

FIG. 81 is a line graph depicting some embodiments of NFL levels in patients treated with TB006 or placebo.

FIG. 82 is a chart depicting some embodiments of study design, treatment schema, and endpoints for parts 1b and 2a of a multi-center, double-blinded followed by an Open Label Extension (OLE) study.

FIG. 83A is a line graph depicting the change in CDR-SB score of subject's with mild to severe AD treated with an anti-Gal3 antibody beginning at Part-Two of a seamless phase 1b/2a double-blind, randomized, multiple dose, multi-center, sequential dose-escalation study through the first battery of cognition, quality of life tests and biomarker testing.

FIG. 83B is a line graph depicting the aggregate change of the CDR-SB score of patients with mild to severe AD following the first battery of cognition, quality of life tests and biomarker testing at approximately three months into an open-label extension study.

FIG. 83C is a line graph depicting the change in the global CDR-SB score of individual subjects with mild to severe AD following the first battery of cognition, quality of life tests and biomarker testing at approximately three months into the OLE study.

FIG. 83D is a chart the total number and percentage of subjects individual subjects with mild to severe AD having improved, the same, or worsened cognition as indicated by the change in CDR-SB score from baseline to the first battery of cognition, quality of life tests and biomarker testing at approximately three months into the OLE study.

FIG. 84 is a line graph depicting the change in MMSE score (left Y-axis) and Global CDR-SB score (right Y-axis) of an individual subject with mild to severe AD beginning at Part-Two of a seamless phase 1b/2a double-blind, randomized, multiple dose, multi-center, sequential dose-escalation study through the first battery of cognition, quality of life tests and biomarker testing at approximately three months into an OLE study.

FIG. 85 is an illustrative representation of some embodiments of the detrimental role of Gal-3 in microglia-mediated neuroinflammation and anti-Gal3 antibody suppression of neuroinflammation and dissolution of toxic oligomers.

FIG. 86 is a pair of line graphs depicting some embodiments of the dose dependent increase in Gal-3 plasma levels in subjects with mild to severe AD during phase 1b (left) and phase 2a (right) treated with anti-Gal3 antibody.

FIG. 87 is a Western blot depicting some embodiments of Gal3 promotion of Aβ42 aggregation.

FIG. 88 is an illustrative representation of some embodiments of a study design for testing cognition in an Alzheimer's disease mouse model treated with or without an anti-Gal3 antibody.

FIG. 89 is a pair of line graphs depicting some embodiments of reversal of cognition loss in an Alzheimer's disease mouse model before and after treatment with or without an anti-Gal3 antibody.

FIG. 90 shows representative images of plaque reduction in some embodiments of an Alzheimer's disease mouse model before and after treatment with or without an anti-Gal3 antibody.

FIG. 91 shows representative images of anti-Gal3 antibody promotion of neurogenesis brain tissue in some embodiments of NeuN stained brain tissue from an Alzheimer's disease mouse model before and after treatment with or without an anti-Gal3 antibody that has been stained for neuronal markers.

FIG. 92 shows representative images of anti-Gal3 antibody reduction of microhemorrhages in Prussian blue stained brain tissue in some embodiments of brain tissue from an Alzheimer's disease mouse model before and after treatment with or without an anti-Gal3 antibody.

FIG. 93 shows representative images of anti-Gal3 antibody reduction of microglial activation in brain tissue in some embodiments of IBA-1 stained brain tissue from an Alzheimer's disease mouse model before and after treatment with or without an anti-Gal3 antibody.

FIG. 94 is a trio of plots showing the change in CDR score in subjects having decreased (disease reversing), stabilized, or increased (disease worsening) CDR scores in some embodiments of subjects treated with an anti-Gal3 antibody.

FIG. 95 is a line graph showing a dose dependent increase in plasma Gal3 levels in subjects treated with 0, 140, 420, or 1000 mg anti-Gal3 antibody.

FIG. 96 is a line graph showing a significant increase in plasma Gal3 levels in subjects treated with 1000 mg anti-Gal3 antibody.

FIG. 97 is a line graph showing a reduction in Aβ plasma levels subjects treated with anti-Gal3 antibodies.

FIG. 98 depicts Gal3 enrichment on blood vessels around plaques in the brain tissue of human and mice subjects.

FIG. 99 is a flowchart depicting some embodiments of a method of treating microglia mediated inflammation.

In some embodiments, any one or more of the administration points depicted in any one of the above figures can be used as a time point of administration (single or recurring) for any of the methods provided herein. In some embodiments, any of the data result (intermediate and/or ultimate) depicted in any one of more of the figures above can be used as an end point to be achieved for the relevant result for any of the methods provided herein.

### DETAILED DESCRIPTION

AD is a chronic progressive neurodegenerative disorder and is the leading cause of dementia among older adults. In the US, AD is one of the leading causes of death, ranking sixth among US adults and fifth among adults aged 65 years or older (CDC, 2020). The most common and thoroughly investigated hypotheses proposed to explain the pathophysiology of AD are the amyloid hypothesis and tau hypothesis. Considerable evidence suggests that toxic changes in Aβ and tau initiate the disease cascade. Abnormal tau accumulates to form neurofibrillary tangles inside nerve bodies, where they destroy cell structure and interfere with neuronal function and communication. Eventually, cell death and brain atrophy result. Other factors that may also contribute to AD include insufficient blood and nutrient delivery of the vascular system to the brain and neuroinflammation which damages neurons in the brain. Drug development in the dementia arena has been highly active in the past 20 years. There are approximately 5 compounds with market approval, all of which aim to improve the symptoms of dementia. These ‘symptomatic” treatments may improve cognition in the short term but have no effect on the progression of the disease. More recently, therapies with large molecule antibodies have explored the impact of targeting the underlying pathology such as amyloid or damaged tau.

These treatments seek to modify the course of the disease by slowing disease progression. Although some of these compounds have shown efficacy in patient subtypes and have demonstrated an acceptable safety and tolerability profile in clinical studies (Panza et al, 2019; Van Dyck, 2017), none have met their primary endpoint; ergo none have been approved for marketing. Therefore, an urgent unmet medical need remains for the treatment of AD.

In some embodiments, it can be useful to identify patients early in the course of their disease for treatment intervention. Patients with advanced symptomatic disease have significant demyelination, neuronal cell loss, and loss of volume. Early identification measures include advanced imaging, most notably MRI, CSF testing for the presence of abnormal Aβ and tau, and sensitivity cognition testing (NIA, 2020; Risacher and Saykin, 2013).

Galectins are a ubiquitous group of proteins found in a variety of cells and tissues and are involved in numerous metabolic processes and functions. At least 15 galectin isotypes have been identified in epithelial cells of the respiratory system, digestive tract, urinary tract, skin, cardiovascular system, liver, immune cells, and in the CNS. Gal-3 plays an important role in different pathogenic conditions, including in neuroinflammatory and neurodegenerative disorders such as multiple sclerosis, AD, Parkinson's disease, and Huntington's disease. In addition, Gal-3 also plays a protective role due to its anti-apoptotic effect in target cells. In vivo data from several investigators suggest that Gal-3 may be an important therapeutic target in pathological conditions including the disorders of the CNS.

TB006 is a humanized IgG4 (S228P) type monoclonal antibody that is highly specific and has high affinity to human Gal-3. It has demonstrated the potential to treat neurodegenerative disorders such as AD and traumatic brain injury, with an apparent wide safety margin, in preclinical models.

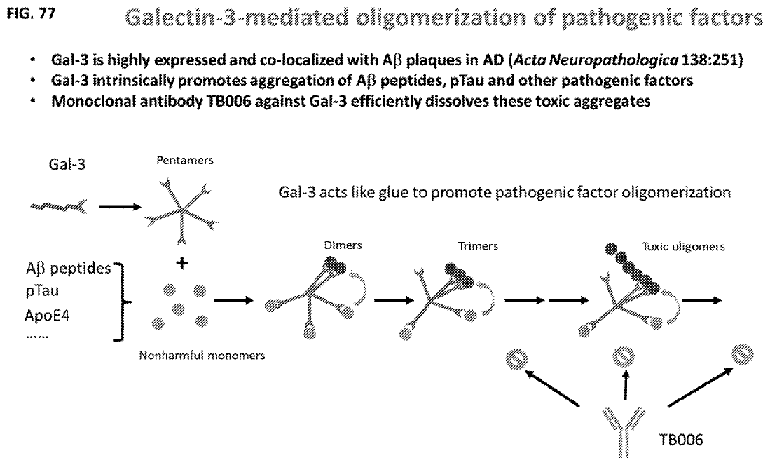

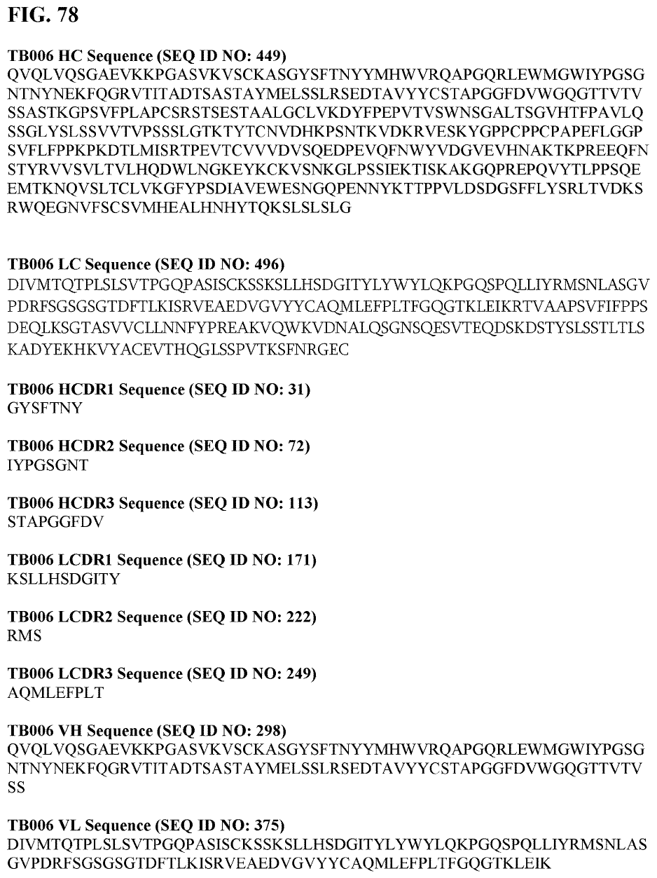

FIG. 78 depicts some exemplary embodiments of TB006 light chain and heavy chain nucleic acid sequences.

In some embodiments, the TB006 antibody comprises the light chain and/or heavy chain in FIG. 78.

In some embodiments, the antibody is formulated as a therapeutic solution. In some embodiments, the therapeutic solution is a clear to slightly opalescent, sterile solution for injection. In some embodiments, the antibody is administered at a unit dose strength of 20 mg/mL (in some embodiments, the 20 mg/ml can be diluted to 8 mg/ml in a total of 500 mL prior to I. V. Administration) in 8 mL vial (160 mg total). In some embodiments, prior to administration, the dosage is added to 300 mL of sterile normal saline for injection, USP, for a total of 500 mL, then infused over 1 hour q28 day±5 days. In some embodiments, the antibody, formulation, therapeutic solution, is administered by IV infusion over 1 hour with the use of a 0.2-micron filter. In some embodiments, the concentration of antibody is 8 mg/mL, in 500 mL per dose.

In some embodiments, a therapeutic formulation comprising TB006 antibody at 20 mg/mL (in some embodiments, the 20 mg/ml can be diluted to 8 mg/ml in a total of 500 mL prior to I. V. administration), 20 mM L-Histidine, 5 mM Methionine, 100 mM NaCl, 0.02% PS80, and a pH of 5.8 is administered to the subject monthly via intravenous injection over the course of 1-hour for a total administered dose of 4000 mg.

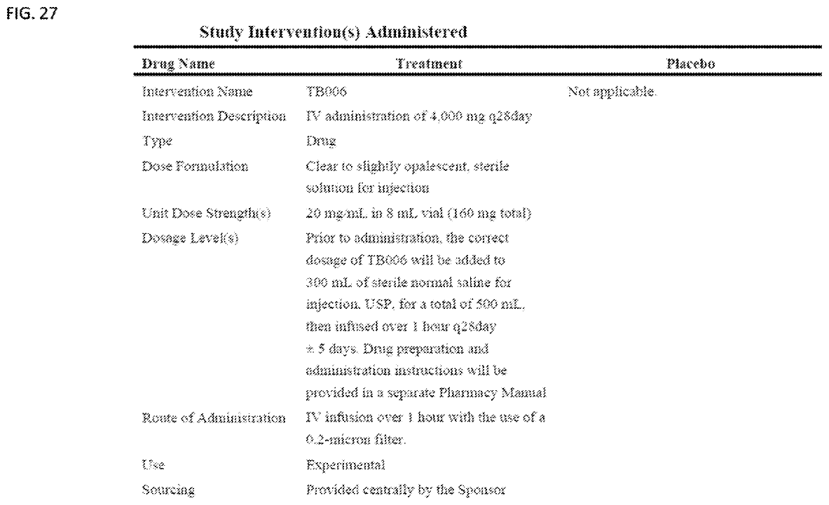

As shown herein and in the examples, in some embodiments, the antibody and/or antibody formulations provided herein can be used in the treatment and/or prevention of AD and other disorders provided herein (such as reducing intracranial volume, reducing alpha-beta 42 plasma, increasing MMSE, reducing: Memory and/or Orientation and/or Judgement and Problem Solving and/or Community Affairs and/or Home and Hobbies and/or Personal care CDRD-SB subdomain based on MMRM). In some embodiments, the antibodies (including TB006 or antibodies having such amino acid components) and/or formulations thereof (optionally including specific amounts of the antibody) can be administered according to the dosing schedule of any one of the dosing schedules described herein, including the frequencies depicted in any one of the schedules for administration depicted in the figures (including, for example, figures, such as FIGS. 82, 83A, 64, 59, 58, 27, In some embodiments, the treatment is given on an initial weekly basis, via IV, for 1, 2, 3, 4, 5, or 6 weeks. In some embodiments, an IV infusion is given at D1, D8, D15, D22, and/or D29. In some embodiments, the initial treatment is a one month treatment of 5 doses. In some embodiments, the amount of the antibody/formulation used is 4000 mg q28 day. In some embodiments, 160 mg of antibody is used (in a total of 500 ml) and infused over an hour, by IV, q28 day±5 days. In some embodiments, 8 mg/ml of Ab is used, in 500 mL, administered every week for five weeks, over about one month. In some embodiments, the concentration of the antibody used is 8 mg/ml, in 500 ml per administration.

In some embodiments, the effectiveness of the treatment is at least as long as any of the time points shown in any of the figures and/or data provided herein. In some embodiments, the effectiveness is for at least 1, 2, 3, 4, 5, or 6 or more months or at least 1 or 2 years. In some embodiments, the benefit occurs at 15, 36, 64, and/or 104 days. In some embodiments, the effectiveness is for at least 101 to 113 weeks. In some embodiments, the antibody is effective for as long as the subject takes the therapy.

### Definitions

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

The section headings used herein are for organizational purposes only and are not to be construed as limiting the subject matter described.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The terms “polypeptide”, “peptide”, and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component.

As used herein the term “amino acid” refers to either natural and/or unnatural or synthetic amino acids, including glycine and both the D or L optical isomers, and amino acid analogs and peptidomimetics.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence. Peptide sequences having at least 80%, 85%, 90%, 95%, 99%, or 100% homology to any one of the peptide sequences disclosed herein and having the same or similar functional properties are envisioned. The percent homology may be determined according to amino acid substitutions, deletions, or additions between two peptide sequences. Peptide sequences having some percent homology to any one of the peptide sequences disclosed herein may be produced and tested by one skilled in the art through conventional methods. The % homology or % identity of two sequences is well understood in the art and can be calculated by the number of conserved amino acids or nucleotides relative to the length of the sequences.

As used herein, the term “antibody” denotes the meaning ascribed to it by one of skill in the art, and further it is intended to include any polypeptide chain-containing molecular structure with a specific shape that fits to and recognizes an epitope, where one or more non-covalent binding interactions stabilize the complex between the molecular structure and the epitope. Antibodies utilized in the present invention may be polyclonal antibodies, although monoclonal antibodies are preferred because they may be reproduced by cell culture or recombinantly and can be modified to reduce their antigenicity.

In addition to entire immunoglobulins (or their recombinant counterparts), immunoglobulin fragments or “binding fragments” comprising the epitope binding site (e.g., Fab′, F(ab′)₂, single-chain variable fragment (scFv), diabody, minibody, nanobody, single-domain antibody (sdAb), or other fragments) are useful as antibody moieties in the present invention. Such antibody fragments may be generated from whole immunoglobulins by ricin, pepsin, papain, or other protease cleavage. Minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif). Nanobodies or single-domain antibodies can also be derived from alternative organisms, such as dromedaries, camels, llamas, alpacas, or sharks. In some embodiments, antibodies can be conjugates, e.g. pegylated antibodies, drug, radioisotope, or toxin conjugates. Monoclonal antibodies directed against a specific epitope, or combination of epitopes, will allow for the targeting and/or depletion of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (e.g. U.S. Pat. No. 5,985,660, hereby expressly incorporated by reference in its entirety).

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As known in the art, a “constant region” of an antibody refers to the constant region of the antibody light chain or the constant region of the antibody heavy chain, cither alone or in combination.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196 (4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the IMGT approach (Lefranc et al., 2003) Dev Comp Immunol. 27:55-77), computational programs such as Paratome (Kunik et al., 2012, Nucl Acids Res. W521-4), the AbM definition, and the conformational definition.

The Kabat definition is a standard for numbering the residues in an antibody and is typically used to identify CDR regions. See, e.g., Johnson & Wu, 2000, Nucleic Acids Res., 28:214-8. The Chothia definition is similar to the Kabat definition, but the Chothia definition takes into account positions of certain structural loop regions. See, e.g., Chothia et al., 1986, J. Mol. Biol., 196:901-17; Chothia et al., 1989, Nature, 342:877-83. The AbM definition uses an integrated suite of computer programs produced by Oxford Molecular Group that model antibody structure. See, e.g., Martin et al., 1989, Proc Natl Acad Sci (USA), 86:9268-9272; “AbM.TM., A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, IMGT, Paratome, AbM, and/or conformational definitions, or a combination of any of the foregoing.

The term “compete,” as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, and/or more rapidly, and/or with greater duration and/or with greater affinity with a particular cell or substance than it does with alternative cells or substances. An antibody “specifically binds” or “preferentially binds” to a target if it binds with greater affinity, and/or avidity, and/or more readily, and/or with greater duration than it binds to other substances. For example, an antibody that specifically or preferentially binds to a CFD epitope is an antibody that binds this epitope with greater affinity, and/or avidity, and/or more readily, and/or with greater duration than it binds to other CFD epitopes or non-CFD epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, the term “antigen binding molecule” refers to a molecule that comprises an antigen binding portion that binds to an antigen and, optionally, a scaffold or framework portion that allows the antigen binding portion to adopt a conformation that promotes binding of the antigen binding portion or provides some additional properties to the antigen binding molecule. In some embodiments, the antigen is Gal3. In some embodiments, the antigen binding portion comprises at least one CDR from an antibody that binds to the antigen. In some embodiments, the antigen binding portion comprises all three CDRs from a heavy chain of an antibody that binds to the antigen or from a light chain of an antibody that binds to the antigen. In some embodiments, the antigen binding portion comprises all six CDRs from an antibody that binds to the antigen (three from the heavy chain and three from the light chain). In some embodiments, the antigen binding portion is an antibody fragment.

Non-limiting examples of antigen binding molecules include antibodies, antibody fragments (e.g., an antigen binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)₂ fragment, a Fv fragment, a Fd fragment, and a complementarity determining region (CDR) fragment. These molecules can be derived from any mammalian source, such as human, mouse, rat, rabbit, pig, dog, cat, horse, donkey, guinea pig, goat, or camelid. Antibody fragments may compete for binding of a target antigen with an intact antibody and the fragments may be produced by the modification of intact antibodies (e.g. enzymatic or chemical cleavage) or synthesized de novo using recombinant DNA technologies or peptide synthesis. The antigen binding molecule can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding molecule as well as wholly synthetic scaffolds comprising, for example, a biocompatible polymer. See, for example, Korndorfer et al., 2003, Proteins: Structure, Function, and Bioinformatics, Volume 53, Issue 1:121-129 (2003); Roque et al., Biotechnol. Prog. 20:639-654 (2004). In addition, peptide antibody mimetics (“PAMs”) can be used, as well as scaffolds based on antibody mimetics utilizing fibronectin components as a scaffold.

An antigen binding molecule can also include a protein comprising one or more antibody fragments incorporated into a single polypeptide chain or into multiple polypeptide chains. For instance, antigen binding molecule can include, but are not limited to, a diabody (see, e.g., EP 404,097; WO 93/11161; and Hollinger et al., Proc. Natl. Acad. Sci. USA, Vol. 90:6444-6448, 1993); an intrabody; a domain antibody (single VL or VH domain or two or more VH domains joined by a peptide linker; see Ward et al., Nature, Vol. 341:544-546, 1989); a maxibody (2 scFvs fused to Fc region, see Fredericks et al., Protein Engineering, Design & Selection, Vol. 17:95-106, 2004 and Powers et al., Journal of Immunological Methods, Vol. 251:123-135, 2001); a triabody; a tetrabody; a minibody (scFv fused to CH3 domain; see Olafsen et al., Protein Eng Des Sel., Vol. 17:315-23, 2004); a peptibody (one or more peptides attached to an Fc region, see WO 00/24782); a linear antibody (a pair of tandem Fd segments (VH-CH1-VH-CH1) which, together with complementary light chain polypeptides, form a pair of antigen binding regions, see Zapata et al., Protein Eng., Vol. 8:1057-1062, 1995); a small modular immunopharmaceutical (see U.S. Patent Publication No. 20030133939); and immunoglobulin fusion proteins (e.g. IgG-scFv, IgG-Fab, 2scFv-IgG, 4scFv-IgG, VH-IgG, IgG-VH, and Fab-scFv-Fc).

In certain embodiments, an antigen binding molecule can have, for example, the structure of an immunoglobulin. An “immunoglobulin” is a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Unless otherwise specified, the complementarity defining regions disclosed herein follow the IMGT definition. In some embodiments, the CDRs can instead by Kabat, Chothia, or other definitions accepted by those of skill in the art.

As used herein, the terms “treating” or “treatment” (and as well understood in the art) means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the recurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some embodiments, chronic administration may be required.

The terms “effective amount” or “effective dose” as used herein have their plain and ordinary meaning as understood in light of the specification, and refer to that amount of a recited composition or compound that results in an observable designated effect. Actual dosage levels of active ingredients in an active composition of the presently disclosed subject matter can be varied so as to administer an amount of the active composition or compound that is effective to achieve the designated response for a particular subject and/or application. The selected dosage level can vary based upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein. In some non-limiting examples, an effective amount or effective dose of a composition or compound may relate to the amount or dose that provides a significant, measurable, or sufficient therapeutic effect towards the treatment of a neurological disease or proteopathy, such as Alzheimer's disease, or a symptom thereof. In some embodiments, the effective amount or effective dose of a composition or compound may treat, ameliorate, or prevent the progression of memory loss, dementia, disorientation, or any other symptom of Alzheimer's disease.

The term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a first compound described herein is administered at the same time, just prior to, or just after the administration of a second compound described herein.

As used herein, the term “therapeutic target” refers to a gene or gene product that, upon modulation of its activity (e.g., by modulation of expression, biological activity, and the like), can provide for modulation of the disease phenotype. As used throughout, “modulation” is meant to refer to an increase or a decrease in the indicated phenomenon (e.g., modulation of a biological activity refers to an increase in a biological activity or a decrease in a biological activity).

As used herein, the term “standard of care”, “best practice” and “standard therapy” refers to the treatment that is accepted by medical practitioners to be an appropriate, proper, effective, and/or widely used treatment for a certain disease. The standard of care of a certain disease depends on many different factors, including the biological effect of treatment, region or location within the body, patient status (e.g. age, weight, gender, hereditary risks, other disabilities, secondary conditions), toxicity, metabolism, bioaccumulation, therapeutic index, dosage, and other factors known in the art. Determining a standard of care for a disease is also dependent on establishing safety and efficacy in clinical trials as standardized by regulatory bodies such as the US Food and Drug Administration, International Council for Harmonization, Health Canada, European Medicines Agency, Therapeutics Goods Administration, Central Drugs Standard Control Organization, National Medical Products Administration, Pharmaceuticals and Medical Devices Agency, Ministry of Food and Drug Safety, and the World Health Organization. The standard of care for a disease may include but is not limited to surgery, radiation, chemotherapy, targeted therapy, or immunotherapy (e.g. PD1/PDL1 or CTLA4 blockade therapy).

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or carrier can refer to a diluent, adjuvant, excipient, or vehicle with which the pharmaceutical formulation is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include sugars, starch, glucose, fructose, lactose, sucrose, maltose, gelatin, malt, rice, flour, chalk, silica gel, sodium stearate, glycerol monostearate, talc, salts, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as glycerol, erythritol, threitol, arabitol, xylitol, ribitol, mannitol, sorbitol, galactitol, fucitol, iditol, inositol, isomalt, maltitol, or lactitol, salt-forming counterions such as sodium, and nonionic surfactants such as TWEEN®, polyethylene glycol (PEG), and PLURONICS®. The formulation, if desired, can also contain minor amounts of wetting, bulking, emulsifying agents, or pH buffering agents. These formulations can take the form of solutions, suspensions, emulsion, sustained release formulations and the like. The formulation should suit the mode of administration.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), tris(hydroxymethyl)aminomethane (Tris), citric acid, ascorbic acid, acetic acid, salts, phosphates, citrates, acetates, succinates, chlorides, bicarbonates, borates, sulfates, sodium chloride, sodium bicarbonate, sodium phosphate, sodium borate, sodium citrate, potassium chloride, potassium phosphate, magnesium sulfate sugars, dextrose, dextran 40, fructose, mannose, lactose, trehalose, galactose, sucrose, sorbitol, mannitol, cellulose, serum, amino acids, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, polysorbate 20, polysorbate 40, polysorbate, 60, polysorbate 80, poloxamer, poloxamer 188, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in the formulation at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

As used herein, the term “neurological disorder” refers to a disease affecting the central and/or peripheral nervous system of a patient. A neurological disorder has a physical cause, such as external or internal mechanical trauma (e.g. stroke or concussion), biological trauma (e.g. infection), chemical trauma (e.g. toxins or drugs), aging and age-related senescence, genetics, and many other causes. Some neurological disorders are caused by the effect or accumulation of mutated or misfolded proteins. These diseases may involve the death of neurons or other cell types associated with the nervous system. Non-limiting examples of neurological disorders include inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer, or otherwise known by a person skilled in the art. Some neurological disorders can also be categorized as protcopathics.

As used herein, the term “proteopathy” refers to a disease which is caused by abnormal folding or accumulation of proteins. An abnormal protein may gain a toxic function, or lose their normal function. It is possible that misfolded proteins can induce the misfolding of otherwise normally folded proteins, resulting in an amplification of the disease (e.g. prion disease). Some non-limiting examples of proteopathies include Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, tauopathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Friedreich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or otherwise known by a person skilled in the art.

As used herein, the term “Alzheimer's disease” refers to the neurodegenerative disease that causes loss of memory, dementia, and eventual death. The cause of Alzheimer's disease is multifactorial and is not fully understood. A common hypothesized cause is the aggregation and accumulation of amyloid beta (Aβ) peptides in the brain, resulting in neuronal degeneration and inflammation. Alzheimer's disease is currently uncurable, and currently available therapeutics, such as cholinesterase inhibitors and NDMA receptor antagonists, have minimal effect on the progression of the disease and/or only treat secondary symptoms of the disease. Applicant has previously shown that anti-Gal3 antibodies have an effect on reducing Aβ oligomerization and is potentially an Alzheimer's disease therapeutic.

As used herein, the term “dementia” refers to a general term for loss of memory, language, problem-solving and other thinking abilities that are severe enough to interfere with daily life. Alzheimer's is the most common cause of dementia. Disorders grouped under the general term “dementia” are caused by abnormal brain changes. These changes trigger a decline in thinking skills, also known as cognitive abilities, severe enough to impair daily life and independent function. They also affect behavior, feelings and relationships. Types of dementia include Alzheimer's disease, vascular dementia, Dementia With Lewy Bodies (DLB), Parkinson's Disease Dementia. Mixed Dementia, Frontotemporal Dementia (FTD), Huntington's Disease, Creutzfeldt-Jakob Disease, normal pressure hydrocephalus, and Wernicke-Korsakoff Syndrome. Alzheimer's disease accounts for 60-80% of cases. Vascular dementia, which occurs because of microscopic bleeding and blood vessel blockage in the brain, is the second most common cause of dementia. Those who experience the brain changes of multiple types of dementia simultaneously have mixed dementia. There are many other conditions that can cause symptoms of dementia, including some that are reversible, such as thyroid problems and vitamin deficiencies. Signs of dementia can vary greatly. Examples include problems with: short-term memory; keeping track of a purse or wallet; paying bills; planning and preparing meals; remembering appointments; and/or traveling out of the neighborhood.

Dementia is caused by damage to brain cells. This damage interferes with the ability of brain cells to communicate with each other. When brain cells cannot communicate normally, thinking, behavior and feelings can be affected. Different types of dementia are associated with particular types of brain cell damage in particular regions of the brain. For example, in Alzheimer's disease, high levels of certain proteins inside and outside brain cells make it hard for brain cells to stay healthy and to communicate with each other. The brain region called the hippocampus is the center of learning and memory in the brain, and the brain cells in this region are often the first to be damaged. That's why memory loss is often one of the earliest symptoms of Alzheimer's. While most changes in the brain that cause dementia are permanent and worsen over time, thinking and memory problems caused by the following conditions may improve when the condition is treated or addressed: depression; medication side effects; excess use of alcohol; thyroid problems; vitamin deficiencies; diagnosis of dementia.

There is no one test to determine if someone has dementia. Doctors diagnose Alzheimer's and other types of dementia based on a careful medical history, a physical examination, laboratory tests, and the characteristic changes in thinking, day-to-day function and behavior associated with each type. Doctors can determine that a person has dementia with a high level of certainty. But it's harder to determine the exact type of dementia because the symptoms and brain changes of different dementias can overlap. In some cases, a doctor may diagnose “dementia” and not specify a type. If this occurs, it may be necessary to see a specialist such as a neurologist, psychiatrist, psychologist or geriatrician. The brain has many distinct regions, each of which is responsible for different functions (for example, memory, judgment and movement). When cells in a particular region are damaged, that region cannot carry out its functions normally.

As used herein, the terms “Cognition Tests” and “Quality of Life Assessments” refer to a battery of cognition and quality of life tests performed on a subject or patient, including the Clinical Dementia Rating Scale-Sum of Boxes (CDR-SB), Cognitive Drug Research battery, Mini-Mental State Examination Scale (MMSE), Neuropsychiatry Inventory (NPI), and the EuroQol 5-Dimension 5-Level Health Related Quality of Life Scale (EQ-5D-5L) throughout the study to assess cognition. Tests may be performed by paper or using an electronic device.

As used herein, the term Mini-Mental State Examination Scale (MMSE) refers to a brief 30-point questionnaire used to assess cognitive impairment with lower scores indicating greater impairment. The Mini-Mental State Examination (MMSE) is the best-known and the most often used short screening tool for providing an overall measure of cognitive impairment in clinical, research and community settings. The MMSE assesses 11 categories of cognition including orientation to time, memory, attention, concentration, naming, repetition, comprehension, and the ability to create a sentence and to copy 2 intersecting polygons. The total scores on the scale ranges from 0 to 30 with lower scores indicating greater impairment.

As used herein, the term Neuropsychiatry Inventory (NPI) refers to rater-administered, fully structured interview in which all questions are provided and read verbatim. The sole source of information is the interview with a caregiver who knows the patient well. This study uses the NPI version with 10 behavioral domains: delusions, hallucinations, agitation/aggression, depression/dysphoria, anxiety, elation/euphoria, apathy/indifference, disinhibition, irritability/lability, and aberrant motor behavior. The NPI total score is calculated by adding the scores of the domains (each domain scores range from 0 to 12). The NPI total score ranges from 0 to 120 with higher scores indicating greater behavioral impairment.

The NPI-D scores in each of the domains are not included in the NPI total score. The NPI-D total score is calculated by adding the scores of caregiver distress in each of the domains (score ranges from 0 to 5 in each domain). The NPI-D total score ranges from 0 to 50 with higher scores indicating greater distress.

As used herein, the term Clinical Dementia Rating Scale-Sum of Boxes (CDR-SB) refers to a global assessment instrument that yields global score and a sum of boxes score. The Clinical Dementia Rating Scale is derived from a semi-structured interview with the participant and an appropriate informant, and it rates impairment in 6 categories (memory, orientation, judgment, and problem solving, community affairs, home and hobbies, and personal care) on a 5-point scale for which 0=no impairment, 0.5=questionable impairment, and 1, 2, and 3=mild, moderate, and severe impairment, respectively. From the 6 individual category ratings, or box scores, the Clinical Dementia Rating Scale-Global Score is established by clinical scoring rules, for which the Clinical Dementia Rating of 0=no dementia and Clinical Dementia Rating of 0.5, 1, 2, or 3=questionable, mild, moderate, or severe dementia, respectively (Morris, 1993). The CDR-SB score is a detailed quantitative general index that provides more information than the Clinical Dementia Rating Scale-Global Score in participants with early (prodromal to mild) dementia (Cedarbaum et al, 2013; Coley et al, 2011). In particular, the CDR-SB has been proposed for use in longitudinal assessment of dementia and is widely used in AD studies as a global measure of disease progression (Williams et al, 2013).

As used herein, the term Cognitive Drug Research battery refers to an automated battery amenable to measurement of cognitive deficits in patients with AD. It consists of performance tasks measuring attention, working memory, episodic memory, and executive function. It contains 11 tests and is performed on a tablet-like device. The average duration of the battery is approximately 25 minutes.

As used herein, the terms “physical” or “physical examination” may refer to assessments of the cardiovascular, respiratory, gastrointestinal, and neurological systems, assessments of the abdomen (liver and spleen), and/or height and weight measurements. In some embodiments a physical examination comprises one or more of the preceding assessments. It will be understood that term contemplates any assessment commonly understood to be part of a physical examination. It will be understood that term contemplates any assessment commonly understood to be part of a neurological examination.

As used herein, the term “neurological examination” may assess mental status, motor and sensory skills, hearing and speech, vision, coordination, and balance.

As used herein, the term “vital signs” refers to temperature, oral temperature, skin temperature, heart rate, respiratory rate, blood pressure, and orthostatic blood pressure, or any assessment or measurement commonly understood to be a measure of a subject or patient's vitality.

As used herein, the term “adverse event” refers to any untoward medical occurrence in a clinical study/patient, temporally associated with the use of study intervention, whether or not considered related to the study intervention. An AE can therefore be any unfavorable and unintended sign (including an abnormal laboratory finding), symptom, or disease (new or exacerbated) temporally associated with the use of study intervention.

As used herein, the term “unsolicited AE” refers to an AE that was not solicited using a patient diary and that is communicated by a patient/patient's parent(s)/legally authorized representative (LAR) who has signed the informed consent. Unsolicited AEs include serious and nonserious AEs. Potential unsolicited AEs may be medically attended (i.e., symptoms or illnesses requiring a hospitalization, emergency room visit, or visit to/by a healthcare provider). The patients/patient's parent(s)/LAR(s) is instructed to contact the site as soon as possible to report medically attended event(s), as well as any events that, though not medically attended, are of patient/patient's parent(s)/LAR(s) concern. Detailed information about reported unsolicited AEs is collected by qualified site personnel and documented in the patient's records.

Unsolicited AEs that are not medically attended nor perceived as a concern by the patient/patient's parent(s)/LAR(s) are collected during an interview with the patients/patient's parent(s)/LAR(s) and by review of available medical records at the next visit.

As used herein, the term “solicited AE” refers to predefined local at the injection site and systemic events for which the patient is specifically questioned, and which are noted by the patient in their diary.

Events meeting the definition of AE include any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment of the investigator (i.e., not related to progression of underlying disease, or more severe than expected for the patient's condition). Exacerbation of a chronic or intermittent pre-existing condition including either an increase in frequency and/or intensity of the condition. New condition detected or diagnosed after administration of a pharmaceutical or therapeutic even though it may have been present before the start of the administration. Signs, symptoms, or the clinical sequelae of a suspected drug-drug interaction. Signs, symptoms, or the clinical sequelae of a suspected overdose of either study intervention or a concomitant medication. Overdose per se is not considered itself as an AE/Serious Adverse Event (SAE) unless it is an intentional overdose taken with possible suicidal/self-harming intent.

Events not Meeting the AE Definition include any clinically significant abnormal laboratory findings or other abnormal safety assessments that are associated with the underlying disease, unless judged to be more severe than expected for the patient's condition. The disease/disorder being studied or expected progression, signs, or symptoms of the disease/disorder being studied, unless more severe than expected for the patient's condition. Medical or surgical procedure (e.g., endoscopy, appendectomy): the condition that leads to the procedure is the AE. Situations in which an untoward medical occurrence did not occur (social and/or convenience admission to a hospital). Anticipated day-to-day fluctuations of pre-existing disease(s) or condition(s) present or detected at the start of the study that do not worsen.

As used herein the term “serious adverse event” (SAE) refers to any untoward medical occurrence that, at any dose, results in death or is life threatening, requires inpatient hospitalization or prolongation of existing hospitalization. In general, hospitalization signifies that the patient has been admitted (usually involving at least an overnight stay) at the hospital or emergency ward for observation and/or treatment that would not have been appropriate in the physician's office or outpatient setting. Complications that occur during hospitalization are AEs. If a complication prolongs hospitalization or fulfills any other serious criteria, the event is serious. When in doubt as to whether hospitalization occurred or was necessary, the AE should be considered serious. Hospitalization for elective treatment of a pre-existing condition that did not worsen from baseline is not considered an AE. Results in persistent or significant disability/incapacity are a SAE. Congenital anomaly/birth defects are SAEs. This definition is not intended to include experiences of relatively minor medical significance such as uncomplicated headache, nausea, vomiting, diarrhea, influenza, and accidental trauma (e.g., sprained ankle) that may interfere with or prevent everyday life functions but do not constitute a substantial disruption.

Other situations that may be considered a SAE include, but are not limited to, important medical events that that may not be immediately life-threatening or result in death or hospitalization but may jeopardize the patient or may require medical or surgical intervention to prevent one of the other outcomes listed in the above definition. These events should usually be considered serious. Examples of such events include invasive or malignant cancers, intensive treatment in an emergency room or at home for allergic bronchospasm, blood dyscrasias, convulsions not resulting in hospitalization, or development of intervention dependency or intervention abuse.

As used herein, the term “life threatening” refers to an event in which the patient was at risk of death at the time of the event. It does not refer to an event, which hypothetically might have caused death, if it were more severe.

The intensity for each AE and SAE may be assessed based on a grading of Grade 1 through Grade 5. Grade 1: Mild; asymptomatic or mild symptoms; clinical or diagnostic observations only; intervention not indicated. Grade 2: Moderate; minimal, local or noninvasive intervention indicated; limiting age appropriate instrumental activities of daily living (ADL) (e.g., preparing meals, shopping for groceries or clothes, using the telephone, managing money). Grade 3: Severe or medically significant but not immediately life-threatening; hospitalization or prolongation of hospitalization indicated; disabling; limiting self-care ADL (e.g., bathing dressing and undressing, feeding self, using the toilet, taking medications). An AE that is assessed as severe should not be confused with an SAE. Severe is a category utilized for rating the severity of an event; and both AEs and SAEs can be assessed as severe. An event is defined as “serious” when it meets at least 1 of the predefined outcomes as described in the definition of an SAE, NOT when it is rated as severe. Grade 4: Life-threatening consequences; urgent intervention indicated. Grade 5: Death related to AE.

As used herein, the term “interventional trial” refers to a trial designed to find out more about a particular intervention, or treatment. In some embodiments, people taking part in an interventional trial are put into different treatment groups. In some embodiments, a computer puts people taking part in an interventional trial are put into different treatment groups.

As used herein, the term “observational studies” refers to studies designed to find out what happens to people in different situations. The research team observes the people taking part, but they don't influence what treatments people have. The people taking part aren't put into treatment groups.

As used herein, the term “feasibility studies” refers to studies that are designed to see if it is possible to do the main study. They aim to find out things such as whether patients and doctors are happy to take part, and how long it might take to collect and analyze the information. They don't answer the main research question about how well a treatment works.

As used herein, the term “pilot study” refers to small versions of the main study. Pilot studies help to test that all the main parts of the study work together. They may also help answer the research question. Sometimes the research team include the information collected during the pilot study in the results of the main study.

As used herein, the term “prevention trials” refers to trials that look at whether a particular treatment can help prevent one or more diseases. These trials can be for the general population or for people who have a higher than normal risk of developing a certain disease.

As used herein, the term “screening trials” refers to trails that test people for the early signs of one or more diseases before they have any symptoms. As with prevention trials, screening trials can be for the general population. Or they can be for a group of people who have a higher than normal risk of developing a certain disease. Researchers may plan screening trials to see if new tests are reliable enough to detect particular diseases. Or they may try to find out if there is an overall benefit in detecting the disease early.

As used herein, the term “treatment trials” refers to trials designed to find out more about the safety and efficacy of new treatments. Treatment trials are generally ran in stages. These stages are called phases. The early phases aim to find out more about the safety and side effects of a new treatments. Later phases aim to see if a new treatment works better than the current treatment. For trials that compare two or more treatments, subjects are put into a treatment group at random. This is a randomized trial. Randomized trials are the best way to get reliable information about how well a new treatment works.

As used herein, the term “multi-arm multi-stage” (MAMS) trials refers to trials that have several treatment groups as well as the standard treatment group. Multi-arm multi-stage (MAMS) trials have the same control group all the way through. The other treatment groups can change as the trial goes on. The research team may decide to stop recruiting people to a particular group. The researchers may add new treatment groups as new drugs become available to look at. This means they don't have to design and launch a brand new trial each time they want to research a new treatment. So it helps get results quicker.

Cohort studies, case control studies and cross sectional studies are all types of observational studies.

As used herein, the term “cohort” refers to a group of people.

As used herein, the term “cohort studies” refers to a study that looks at groups of people. A cohort study may follow the group over a period of time. A research team may recruit people who do not have a certain disease or diseases and collect information about them for a number of years. The researchers see who in the group develops the disease and who doesn't. They then look to see whether the people who developed the disease had anything in common. Cohort studies are very useful ways of finding out more about risk factors. But they are expensive and time consuming. They can be used when it wouldn't be possible to test a theory any other way.

As used herein, the term “case control study” refers to the study of a group of people who have a disease (cases) and a group of people who don't (controls). They then look back to see how many people in each group were exposed to a certain risk factor. Researchers want to make the results as reliable as possible. So they try to make sure the people in each group have the same general factors such as age or gender.

As used herein, the term “cross sectional study” refers to a study carried out at one point in time, or over a short period of time. Cross sectional studies aim to determine which subject's have been exposed to a risk factor for a certain disease or disease and who has developed said disease or diseases, and determine if there is a link between the risk factor and development of the disease.

As used herein, the term “disability” means a substantial disruption of a person's ability to conduct normal life functions.

As used herein, the term “microglia” refers to a specialized population of macrophage-like cells in the central nervous system (CNS). Microglia are considered immune sentinels that are the primary innate immune effector cells of the CNS. Microglia are also involved in synaptic organization, trophic neuronal support during development, phagocytosis of apoptotic cells in the developing brain, myelin turnover, control of neuronal excitability, phagocytic debris removal as well as brain protection and repair. Activated microglia have been identified surrounding lesions of various neurodegenerative diseases, such as Alzheimer's disease, Parkinson's disease, muscular amyotrophic lateral sclerosis, and multiple sclerosis.

As used herein, the term “macrophage” refers to a type of white blood cell that surrounds and kills microorganisms, removes dead cells, and stimulates the action of other immune system cells.

As used herein, the term “central nervous system” (CNS) refers to part of the nervous system that consists of the brain and spinal cord.

As used herein, the term “neurogenesis” refers to the process by which new neurons are formed in the brain. Neurogenesis is crucial when an embryo is developing, but also continues in certain brain regions after birth and throughout a subject's lifespan. Altered neurogenesis has been identified in Alzheimer's disease (AD), in both human AD brains and AD rodent models.

The term “% w/w” or “% wt/wt” means a percentage expressed in terms of the weight of the ingredient or agent over the total weight of the composition multiplied by 100.

As used herein, the term “Neurofilament Light chain (NFL) refers to neuronal cytoplasmic proteins highly expressed in large caliber myelinated axons. Motor neuron degeneration: Mutant neurofilament (NF) proteins are characterized by defective transport or assembly and NF aggregation or accumulation, leading to atrophy and motor neuron degeneration. Mutations in neurofilament light (NFL) subunit cause Charcot-Marie-Tooth disease, the most common inherited peripheral neuropathy, and NF mutations have been found in patients with early-onset PD, AD and sporadic Amyotrophic Lateral Sclerosis (ALS). In ALS, aggregation of NFL may also promote aggregation of wildly expressed proteins that are destabilized by missense mutations.

As used herein, the term “single-photon emission computerized tomography” (SPECT) refers to a nuclear imaging modality that produces a 3-dimensional image of the distribution of a radioactive tracer or probe injected into the bloodstream and subsequently taken up by certain tissues.

As used herein, the term “positron emission tomography” (PET) imaging refers to a versatile nuclear medicine technique to investigate the expression of molecular targets noninvasively. PET imaging tracks the spatial distribution of a positron-emitting radionuclide that is typically conjugated to a targeting molecule. The radioligands [¹¹C] PIB and [¹⁸F] florbetaben have been widely used to trace Aβ aggregation in Alzheimer's disease (AD) and mild cognitive impairment (MCI). Furthermore, radioligands targeting translocator protein (TSPO) for PET imaging, such as [¹⁸F] GE180 and [11C] ER176, are employed to detect neuroinflammation in neurodegenerative diseases. Antibody-based PET imaging has been extensively studied in oncology in decades. In neurodegenerative diseases, scientists developed radiolabeling of monoclonal antibodies (mAbs) with positron emitters for PET imaging to study the molecule biodistribution and related therapeutics.

As used herein, the term gamma camera,” also called “a “scintillation camera” or “Anger camera”, refers to an imaging device used to image gamma radiation-emitting radioisotopes. This technique is known as scintigraphy and is used to image and analyze the distribution of gamma-emitting radionuclides medically introduced into the human body. The gamma camera consists of a collimator, a crystal plane, and an array of photomultiplier tubes connected to a computer system. The collimator is typically a single plate of lead or tungsten with many holes through it; this allows only photons traveling parallel to the collimator holes to reach the crystal, which is located behind the collimator. Once photons reach the crystal, they are absorbed into the crystal and this absorbed energy is emitted as flashes of light, a process called scintillation. The brightness of light is proportional to the energy absorbed by the crystal. The light flashes are converted into an electronic signal that is ultimately processed to produce an image. Radionuclides can be ingested or injected into the body. The camera accumulates counts of gamma photons, which are detected by crystals in the camera. Just like an X-ray, the gamma camera will yield a two-dimensional projection of a three-dimensional object. A tomographic version of the gamma camera is called SPECT, which yields slices through the body. Because the detection techniques of gamma cameras and SPECT are based on the same concept, the same radioisotopes can be used for both techniques. Commonly used isotopes include technetium-99m, iodine-123, and indium-111.

As used herein, the term “MRI” refers to a procedure that uses radio waves, a powerful magnet, and a computer to make a series of detailed pictures of areas inside the body. A contrast agent, such as gadolinium, may be injected into a vein to help the tissues and organs show up more clearly in the picture. MRI may be used to help diagnose disease, plan treatment, or find out how well treatment is working. It is especially useful for imaging the brain and spinal cord, the heart and blood vessels, the bones, joints, and other soft tissues, the organs in the pelvis and abdomen, and the breast. Also called magnetic resonance imaging, NMRI, and nuclear magnetic resonance imaging.

As used herein, the term “computed tomography scan” (CT scan) refers to a procedure that uses a computer linked to an x-ray machine to make a series of detailed pictures of areas inside the body. The pictures are taken from different angles and are used to create 3-dimensional (3-D) views of tissues and organs. A dye may be injected into a vein or swallowed to help the tissues and organs show up more clearly. A CT scan may be used to help diagnose disease, plan treatment, or find out how well treatment is working. Also called CAT scan, computed tomography scan, computerized axial tomography scan, and computerized tomography.

As used herein, the term “Morris water maze” (MWM) refers to a test of spatial learning for rodents that is designed to test spatial memory and long term memory by observing and recording escape latency, thigmotaxis duration, distance moved, and velocity while in a water tank. The MWM relies on distal cues to navigate from start locations around the perimeter of an open swimming arena to locate a submerged escape platform. Spatial learning is assessed across repeated trials and reference memory is determined by preference for the platform area when the platform is absent. Reversal and shift trials enhance the detection of spatial impairments. Trial-dependent, latent and discrimination learning can be assessed using modifications of the basic protocol. Search-to-platform area determines the degree of reliance on spatial versus non-spatial strategies. Cued trials determine whether performance factors that are unrelated to place learning are present. Escape from water is relatively immune from activity or body mass differences, making it ideal for many experimental models. The MWM has proven to be a robust and reliable test that is strongly correlated with hippocampal synaptic plasticity and NMDA receptor function. We present protocols for performing variants of the MWM test, from which results can be obtained from individual animals in as few as 6 days.

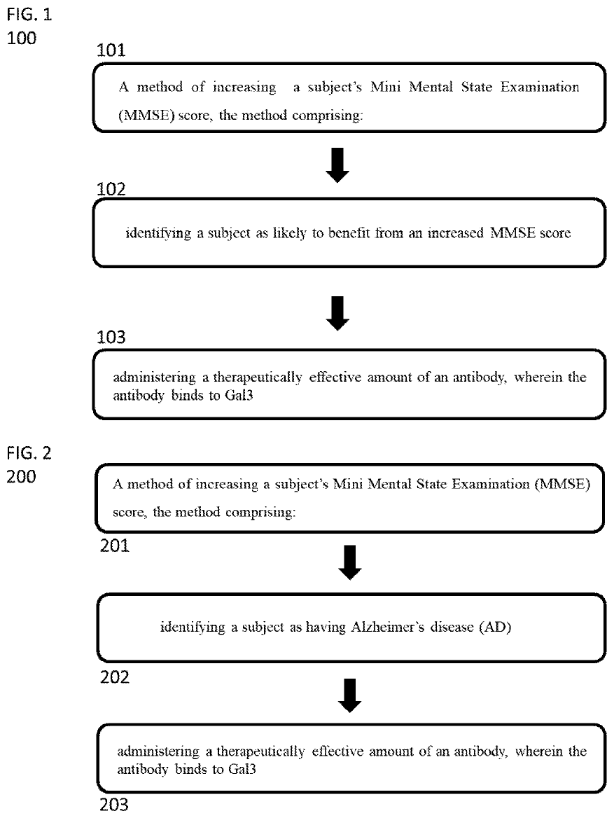

FIG. 1 is a flow chart depicting some embodiments of a method of increasing a subject's Mini Mental State Examination (MMSE) score.

In some embodiments, a method of increasing a subject's Mini Mental State Examination (MMSE) score 101 is disclosed. In some embodiments, the method comprises identifying a subject as likely to benefit from an increased MMSE score 102, and administering a therapeutically effective amount of an antibody, wherein the antibody binds to Gal3 103.

Some embodiments provided herein relate to a method of increasing a subject's Mini Mental State Examination (MMSE) score, the method comprising: identifying a subject as having Alzheimer's disease (AD), and administering a therapeutically effective amount of an antibody, wherein the antibody binds to Gal3.

FIG. 2 is a flow chart depicting some embodiments of a method of increasing a subject's Mini Mental State Examination (MMSE) score.

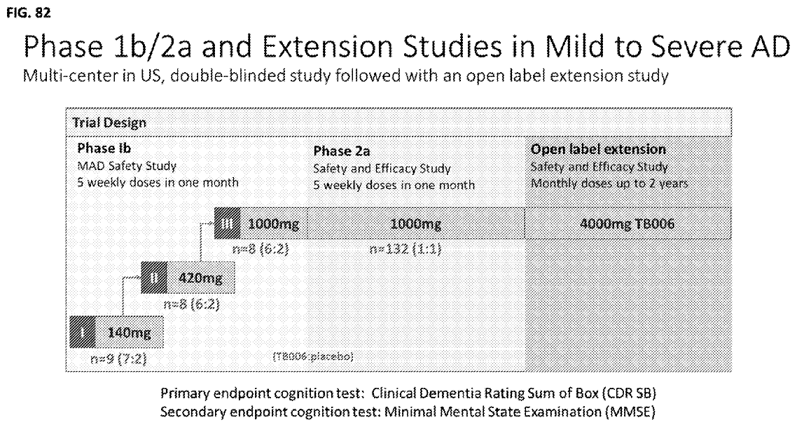

FIG. 82 is a chart depicting some embodiments of study design, treatment schema, and endpoints for parts 1b and 2a of a multi-center, double-blinded followed by an Open Label Extension (OLE) study.

In some embodiments, a method of increasing a subject's Mini Mental State Examination (MMSE) score 201 is disclosed. In some embodiments, the method comprises identifying a subject as having Alzheimer's disease 202, and administering a therapeutically effective amount of an antibody, wherein the antibody binds to Gal3 203.

In some embodiments, a method of increasing a subject's Mini Mental State Examination (MMSE) score is disclosed. In some embodiments, the method comprises identifying a subject as likely to benefit from an increased MMSE score, and administering 140, 420, or 1000 mg of an anti-Gal3 antibody once weekly for 5 weeks. In some embodiments, the antibody is administered 5 times in a month, once weekly at 140 mg. In some embodiments, the antibody is administered 5 times in a month, once weekly at 420 mg. In some embodiments, the antibody is administered 5 times in a month, once weekly at 1000 mg. In some embodiments, following the first month, the antibody is administered 5 times in a month, once weekly at 1000 mg. For example, in some embodiments, the antibody is administered 5 times in a month, once weekly at 140 mg, followed by administration 5 times in a month, once weekly at 1000 mg. In some embodiments, the antibody is administered 5 times in a month, once weekly at 420 mg, followed by administration 5 times in a month, once weekly at 1000 mg. In some embodiments, the antibody is administered 5 times in a month, once weekly at 1000 mg, followed by administration 5 times in a month, once weekly at 1000 mg. In some embodiments, following the first or second month, the antibody is administered once-monthly, about every 28 days at 4000 mg. For example, in some embodiments, the antibody is administered 5 times in a month, once weekly at 140 mg, followed by administration 5 times in a month, once weekly at 1000 mg, followed by administration once-monthly, about every 28 days at 4000 mg. In some embodiments, the antibody is administered 5 times in a month, once weekly at 420 mg, followed by administration 5 times in a month, once weekly at 1000 mg, followed by administration once-monthly, about every 28 days at 4000 mg. In some embodiments, the antibody is administered 5 times in a month, once weekly at 1000 mg, followed by administration 5 times in a month, once weekly at 1000 mg, followed by administration once-monthly, about every 28 days at 4000 mg. In some embodiments, the antibody is administered 5 times in a month, once weekly at 140 mg, followed by administration once-monthly, about every 28 days at 4000 mg. In some embodiments, the antibody is administered 5 times in a month, once weekly at 420 mg, followed by administration once-monthly, about every 28 days at 4000 mg. In some embodiments, the antibody is administered 5 times in a month, once weekly at 1000 mg, followed by administration once-monthly, about every 28 days at 4000 mg. In some embodiments, the treatment continues for about 1, 2, 3, 4, 5, 6, 7, 8, 9, 10, 11, 12, 12, 14, 15, 16, 17, 18, 19, 20, 21, 22, 23, or 24 months, or for a range that is defined by any two of the preceding values. For example, in some embodiments, the treatment continues for between about 1-24, 1-18, 1-12, 1-6, 1-3, 3-24, 3-18, 3-12, 3-6, 6-24, 6-18, 6-12, 12-24, 12-18, or 18-24, months. In some embodiments, the antibody is administered 5 times in a month, once weekly at 140 mg, followed by administration 5 times in a month, once weekly at 1000 mg, followed by administration once-monthly, about every 28 days at 4000 mg, for up to about 2 years. In some embodiments, the antibody is administered 5 times in a month, once weekly at 420 mg, followed by administration 5 times in a month, once weekly at 1000 mg, followed by administration once-monthly, about every 28 days at 4000 mg, for up to about 2 years. In some embodiments, the antibody is administered 5 times in a month, once weekly at 1000 mg, followed by administration 5 times in a month, once weekly at 1000 mg, followed by administration once-monthly, about every 28 days at 4000 mg, for up to about 2 years. In some embodiments, the antibody is administered 5 times in a month, once weekly at 140 mg, followed by administration once-monthly, about every 28 days at 4000 mg, for up to about 2 years. In some embodiments, the antibody is administered 5 times in a month, once weekly at 420 mg, followed by administration once-monthly, about every 28 days at 4000 mg, for up to about 2 years. In some embodiments, the antibody is administered 5 times in a month, once weekly at 1000 mg, followed by administration once-monthly, about every 28 days at 4000 mg, for up to about 2 years. In some embodiments, the treatment continues for the life of the patient. In some embodiments, the antibody is administered 5 times in a month, once weekly at 140 mg, followed by administration 5 times in a month, once weekly at 1000 mg, followed by administration once-monthly, about every 28 days at 4000 mg, for the life of the patient. In some embodiments, the antibody is administered 5 times in a month, once weekly at 420 mg, followed by administration 5 times in a month, once weekly at 1000 mg, followed by administration once-monthly, about every 28 days at 4000 mg, for the life of the patient. In some embodiments, the antibody is administered 5 times in a month, once weekly at 1000 mg, followed by administration 5 times in a month, once weekly at 1000 mg, followed by administration once-monthly, about every 28 days at 4000 mg, for the life of the patient. In some embodiments, the antibody is administered 5 times in a month, once weekly at 140 mg, followed by administration once-monthly, about every 28 days at 4000 mg, for the life of the patient. In some embodiments, the antibody is administered 5 times in a month, once weekly at 420 mg, followed by administration once-monthly, about every 28 days at 4000 mg, for the life of the patient. In some embodiments, the antibody is administered 5 times in a month, once weekly at 1000 mg, followed by administration once-monthly, about every 28 days at 4000 mg, for the life of the patient. In some embodiments, administration of the antibody results in an increase in the subject's MMSE score. In some embodiments, the subject's MMSE score increases by 1, 2, 3, 4, 5, 6, 7, 8, 9, 10, 11, 12, 12, 14, or 15, points, or by a range that is defined by any two of the preceding values. For example, in some embodiments, the subject's MMSE score is increased by about 1-15, 1-10, 1-7, 1-5, 1-3, 3-15, 3-10, 3-7, 3-5, 5-15, 5-10, 5-7, 7-15 7-10, or 10-15, points following one or more administrations of the antibody. In some embodiments, the antibody is administered by IV infusion. In some embodiments, the antibody is administered as a therapeutic formulation. In some embodiments, the antibody is administered by IV as therapeutic formulation comprising 20 mg/mL (in some embodiments, the 20 mg/ml can be diluted to 8 mg/ml in a total of 500 mL prior to I. V. administration) antibody, 20 mM L-Histidine, 5 mM Methionine, 100 mM NaCl, 0.02% PS80, and a pH of 5.8, over the course of 1-hour for a total administered dose of 4000 mg. In some embodiments, the antibody is administered subcutaneously. In some embodiments, the antibody is TB006.

In some embodiments, a method of increasing a subject's Mini Mental State Examination (MMSE) score is disclosed. In some embodiments, the method comprises identifying a subject as likely to benefit from an increased MMSE score, and administering 140, 420, or 1000 mg of TB006 antibody once weekly for 5 weeks. In some embodiments, TB006 is administered 5 times in a month, once weekly at 140 mg. In some embodiments, TB006 is administered 5 times in a month, once weekly at 420 mg. In some embodiments, TB006 is administered 5 times in a month, once weekly at 1000 mg. In some embodiments, following the first month, TB006 is administered 5 times in a month, once weekly at 1000 mg. For example, in some embodiments, TB006 is administered 5 times in a month, once weekly at 140 mg, followed by administration 5 times in a month, once weekly at 1000 mg. In some embodiments, TB006 is administered 5 times in a month, once weekly at 420 mg, followed by administration 5 times in a month, once weekly at 1000 mg. In some embodiments, TB006 is administered 5 times in a month, once weekly at 1000 mg, followed by administration 5 times in a month, once weekly at 1000 mg. In some embodiments, following the first or second month, TB006 is administered once-monthly, about every 28 days at 4000 mg. For example, in some embodiments, TB006 is administered 5 times in a month, once weekly at 140 mg, followed by administration 5 times in a month, once weekly at 1000 mg, followed by administration once-monthly, about every 28 days at 4000 mg. In some embodiments, TB006 is administered 5 times in a month, once weekly at 420 mg, followed by administration 5 times in a month, once weekly at 1000 mg, followed by administration once-monthly, about every 28 days at 4000 mg. In some embodiments, TB006 is administered 5 times in a month, once weekly at 1000 mg, followed by administration 5 times in a month, once weekly at 1000 mg, followed by administration once-monthly, about every 28 days at 4000 mg. In some embodiments, TB006 is administered 5 times in a month, once weekly at 140 mg, followed by administration once-monthly, about every 28 days at 4000 mg. In some embodiments, TB006 is administered 5 times in a month, once weekly at 420 mg, followed by administration once-monthly, about every 28 days at 4000 mg. In some embodiments, TB006 is administered 5 times in a month, once weekly at 1000 mg, followed by administration once-monthly, about every 28 days at 4000 mg. In some embodiments, the treatment continues for about 1, 2, 3, 4, 5, 6, 7, 8, 9, 10, 11, 12, 12, 14, 15, 16, 17, 18, 19, 20, 21, 22, 23, or 24 months, or for a range that is defined by any two of the preceding values. For example, in some embodiments, the treatment continues for between about 1-24, 1-18, 1-12, 1-6, 1-3, 3-24, 3-18, 3-12, 3-6, 6-24, 6-18, 6-12, 12-24, 12-18, or 18-24, months. In some embodiments, TB006 is administered 5 times in a month, once weekly at 140 mg, followed by administration 5 times in a month, once weekly at 1000 mg, followed by administration once-monthly, about every 28 days at 4000 mg, for up to about 2 years. In some embodiments, TB006 is administered 5 times in a month, once weekly at 420 mg, followed by administration 5 times in a month, once weekly at 1000 mg, followed by administration once-monthly, about every 28 days at 4000 mg, for up to about 2 years. In some embodiments, TB006 is administered 5 times in a month, once weekly at 1000 mg, followed by administration 5 times in a month, once weekly at 1000 mg, followed by administration once-monthly, about every 28 days at 4000 mg, for up to about 2 years. In some embodiments, TB006 is administered 5 times in a month, once weekly at 140 mg, followed by administration once-monthly, about every 28 days at 4000 mg, for up to about 2 years. In some embodiments, TB006 is administered 5 times in a month, once weekly at 420 mg, followed by administration once-monthly, about every 28 days at 4000 mg, for up to about 2 years. In some embodiments, TB006 is administered 5 times in a month, once weekly at 1000 mg, followed by administration once-monthly, about every 28 days at 4000 mg, for up to about 2 years. In some embodiments, the treatment continues for the life of the patient. In some embodiments, TB006 is administered 5 times in a month, once weekly at 140 mg, followed by administration 5 times in a month, once weekly at 1000 mg, followed by administration once-monthly, about every 28 days at 4000 mg, for the life of the patient. In some embodiments, TB006 is administered 5 times in a month, once weekly at 420 mg, followed by administration 5 times in a month, once weekly at 1000 mg, followed by administration once-monthly, about every 28 days at 4000 mg, for the life of the patient. In some embodiments, TB006 is administered 5 times in a month, once weekly at 1000 mg, followed by administration 5 times in a month, once weekly at 1000 mg, followed by administration once-monthly, about every 28 days at 4000 mg, for the life of the patient. In some embodiments, the antibody is administered 5 times in a month, once weekly at 140 mg, followed by administration once-monthly, about every 28 days at 4000 mg, for the life of the patient. In some embodiments, TB006 is administered 5 times in a month, once weekly at 420 mg, followed by administration once-monthly, about every 28 days at 4000 mg, for the life of the patient. In some embodiments, TB006 is administered 5 times in a month, once weekly at 1000 mg, followed by administration once-monthly, about every 28 days at 4000 mg, for the life of the patient. In some embodiments, administration of TB006 results in an increase in the subject's MMSE score. In some embodiments, the subject's MMSE score increases by 1, 2, 3, 4, 5, 6, 7, 8, 9, 10, 11, 12, 12, 14, or 15, points, or by a range that is defined by any two of the preceding values. For example, in some embodiments, the subject's MMSE score is increased by about 1-15, 1-10, 1-7, 1-5, 1-3, 3-15, 3-10, 3-7, 3-5, 5-15, 5-10, 5-7, 7-15 7-10, or 10-15, points following one or more administrations of TB006. In some embodiments, TB006 is administered by IV infusion. In some embodiments, TB006 is administered as a therapeutic formulation. In some embodiments, TB006 is administered by IV as therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, over the course of 1-hour for a total administered dose of 4000 mg. In some embodiments, TB006 is administered subcutaneously. In some embodiments, the dose of antibody administered is up to 5000 mg. In some embodiments, the dose of antibody administered is between 70 and 4000 mg. For example, in some embodiments, the dose of antibody administered is 70, 100, 140, 250, 420, 500, 600, 700, 750, 800, 900, 1000, 1250, 1500, 1750, 2000, 2250, 2500, 2750, 3000, 3250, 3500, 3750, 4000, 4250, 4500, 4750, or 5000 mg, or a range that is defined by any two of the preceding values. In some embodiments, the dose administered is chosen to achieve an improved MMSE score. In some embodiments, the dose administered is chosen to achieve an increase in the subject's MMSE score of between 1-15 points.

In some embodiments, a method of increasing a subject's Mini Mental State Examination (MMSE) score is disclosed. In some embodiments, the method comprises identifying a subject as likely to benefit from an increased MMSE score, and administering a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 140 mg once weekly for 5 weeks. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 420 mg once weekly for 5 weeks. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly 5 times in a month. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly 5 times in a month. In some embodiments, following the first month, a therapeutic formulation comprising 20 mg/ml (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly is administered 5 times in a month. For example, in some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 140 mg once weekly is administered 5 times in a month, followed by administration 5 times in a month, once weekly at 1000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 420 mg once weekly is administered 5 times in a month, followed by administration 5 times in a month, once weekly at 1000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly is administered 5 times in a month, followed by administration 5 times in a month, once weekly at 1000 mg. In some embodiments, following the first or second month, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 4000 mg is administered once-monthly, about every 28 days. For example, in some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour for a total administered dose of 140 mg 5 times in a month, followed by administration 5 times in a month, once weekly at 1000 mg, followed by administration once-monthly, about every 28 days at 4000 mg. In some embodiments, a therapeutic formulation comprising 20 mg/ml (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 420 mg once weekly is administered 5 times in a month, once weekly, followed by administration 5 times in a month, once weekly at 1000 mg, followed by administration once-monthly, about every 28 days at 4000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly is administered 5 times in a month, once weekly, followed by administration 5 times in a month, once weekly at 1000 mg, followed by administration once-monthly, about every 28 days at 4000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 140 mg once weekly is administered 5 times in a month, once weekly, followed by administration once-monthly, about every 28 days at 4000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 420 mg is administered 5 times in a month, once weekly, followed by administration once-monthly, about every 28 days at 4000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly is administered 5 times in a month, once weekly, followed by administration once-monthly, about every 28 days at 4000 mg. In some embodiments, the treatment continues for about 1, 2, 3, 4, 5, 6, 7, 8, 9, 10, 11, 12, 12, 14, 15, 16, 17, 18, 19, 20, 21, 22, 23, or 24 months, or for a range that is defined by any two of the preceding values. For example, in some embodiments, the treatment continues for between about 1-24, 1-18, 1-12, 1-6, 1-3, 3-24, 3-18, 3-12, 3-6, 6-24, 6-18, 6-12, 12-24, 12-18, or 18-24, months. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour for a total administered dose of 140 mg, once weekly, 5 times in a month, followed by administration 5 times in a month, once weekly at 1000 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methioninc, 100 mM NaCl, 0.02% PS80, and a pH of 5.8, is administered intravenously over the course of 1-hour for a total administered dose of 420 mg once weekly 5 times in a month, followed by administration 5 times in a month, once weekly at 1000 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 1000 mg, followed by administration 5 times in a month, once weekly at 1000 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 140 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 420 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 1000 mg, followed by administration once-monthly, about every 28 days at 4000 mg, for up to about 2 years. In some embodiments, the treatment continues for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 140 mg, followed by administration 5 times in a month, once weekly at 1000 mg, followed by administration once-monthly, about every 28 days at 4000 mg, for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 420 mg, followed by administration 5 times in a month, once weekly at 1000 mg, followed by administration once-monthly, about every 28 days at 4000 mg, for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 1000 mg, followed by administration 5 times in a month, once weekly at 1000 mg, followed by administration once-monthly, about every 28 days at 4000 mg, for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 140 mg, followed by administration once-monthly, about every 28 days at 4000 mg, for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 420 mg, followed by administration once-monthly, about every 28 days at 4000 mg, for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 1000 mg, followed by administration once-monthly, about every 28 days at 4000 mg, for the life of the patient. In some embodiments, administration of a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, results in an increase in the subject's MMSE score. In some embodiments, the subject's MMSE score increases by 1, 2, 3, 4, 5, 6, 7, 8, 9, 10, 11, 12, 12, 14, or 15, points, or by a range that is defined by any two of the preceding values. For example, in some embodiments, the subject's MMSE score is increased by about 1-15, 1-10, 1-7, 1-5, 1-3, 3-15, 3-10, 3-7, 3-5, 5-15, 5-10, 5-7, 7-15 7-10, or 10-15, points following one or more administrations of a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8. In some embodiments, the dose of antibody administered is between 70 and 4000 mg. For example, in some embodiments, the dose of antibody administered is 70, 100, 140, 250, 420, 500, 600, 700, 750, 800, 900, 1000, 1250, 1500, 1750, 2000, 2250, 2500, 2750, 3000, 3250, 3500, 3750, 4000, 4250, 4500, 4750, or 5000 mg, or a range that is defined by any two of the preceding values. In some embodiments, the dose administered is chosen to achieve an improved MMSE score. In some embodiments, the dose administered is chosen to achieve an increase in the subject's MMSE score of between 1-15 points.

Some embodiments provided herein relate to a method of increasing a subject's EQ-5D-5L score, the method comprising: identifying a subject as likely to benefit from an increased EQ-5D-5L score, and administering a therapeutically effective amount of an antibody, wherein the antibody binds to Gal3.

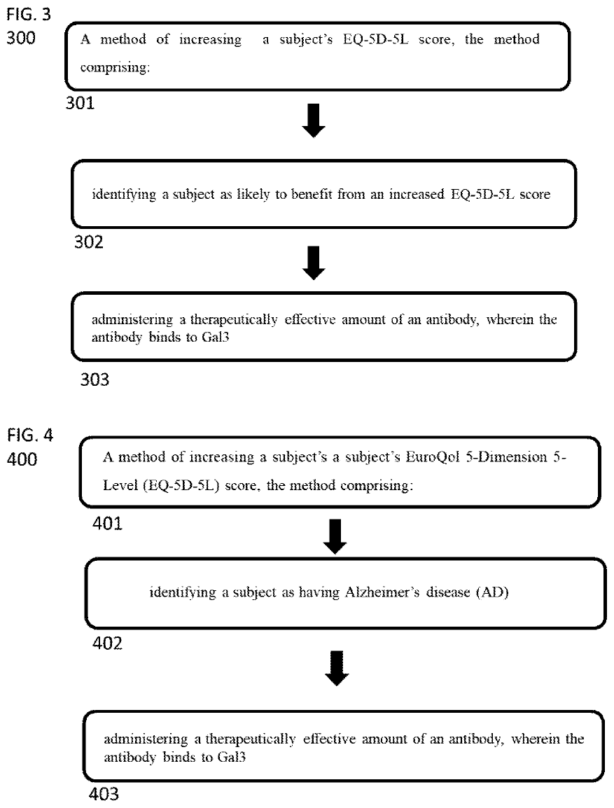

FIG. 3 is a flow chart depicting some embodiments of a method of increasing a subject's a subject's EQ-5D-5L score.

In some embodiments, a method of increasing a subject's a subject's EQ-5D-5L score 301 is disclosed. In some embodiments, the method comprises identifying a subject as likely to benefit from an increased EQ-5D-5L score 302, and administering a therapeutically effective amount of an antibody, wherein the antibody binds to Gal3 303.

Some embodiments provided herein relate to a method of increasing a subject's a subject's EuroQol 5-Dimension 5-Level (EQ-5D-5L) score, the method comprising: identifying a subject as having Alzheimer's disease (AD), and administering a therapeutically effective amount of an antibody, wherein the antibody binds to Gal3.

FIG. 4 is a flow chart depicting some embodiments of a method of increasing a subject's EQ-5D-5L score.

In some embodiments, a method of increasing a subject's EQ-5D-5L score 401 is disclosed. In some embodiments, the method comprises identifying a subject as having Alzheimer's disease 402, and administering a therapeutically effective amount of an antibody, wherein the antibody binds to Gal3 403.

Some embodiments provided herein relate to a method of decreasing a subject's Neuropsychiatric Inventory (NPI) score, the method comprising: identifying a subject as likely to benefit from an increased Neuropsychiatric Inventory (NPI) score, and administering a therapeutically effective amount of an antibody, wherein the antibody binds to Gal3.

FIG. 5 is a flow chart depicting some embodiments of a method of decreasing a subject's Neuropsychiatric Inventory (NPI) score.

In some embodiments, a method of decreasing subject's NPI score 501 is disclosed. In some embodiments, the method comprises identifying a subject as likely to benefit from a decreased NPI score 502, and administering a therapeutically effective amount of an antibody, wherein the antibody binds to Gal3 503.

Some embodiments provided herein relate to a method of decreasing a subject's a subject's Neuropsychiatric Inventory (NPI) score, the method comprising: identifying a subject as having Alzheimer's disease (AD), and administering a therapeutically effective amount of an antibody, wherein the antibody binds to Gal3.

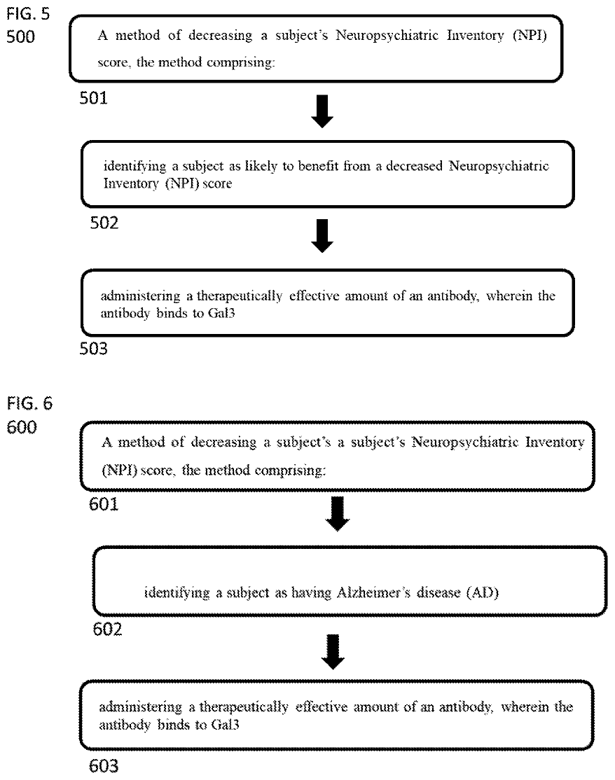

FIG. 6 is a flow chart depicting some embodiments of a method of decreasing a subject's Neuropsychiatric Inventory (NPI) score.

In some embodiments, a method of decreasing a subject's NPI score 601 is disclosed. In some embodiments, the method comprises identifying a subject as having Alzheimer's disease 602, and administering a therapeutically effective amount of an antibody, wherein the antibody binds to Gal3 603.

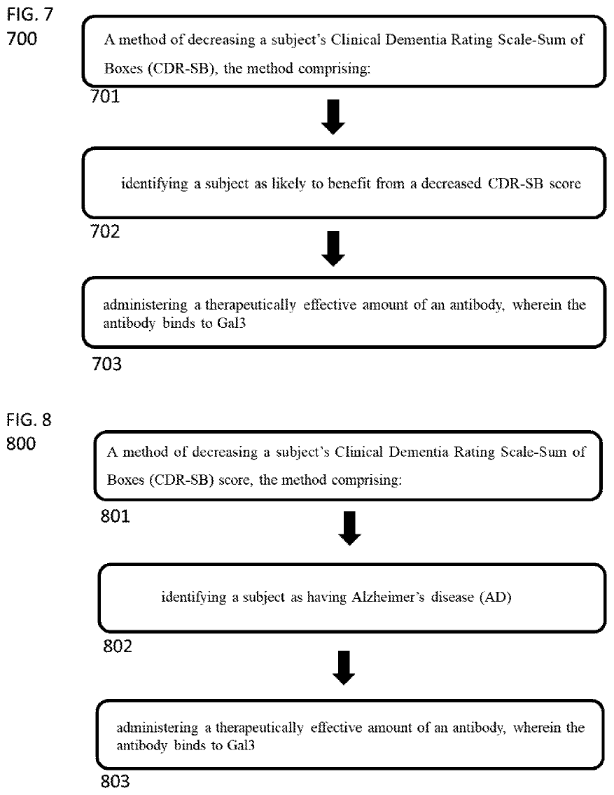

FIG. 7 is a flow chart depicting some embodiments of a method of decreasing a subject's Clinical Dementia Rating Scale-Sum of Boxes (CDR-SB).

In some embodiments, a method of decreasing a subject's a subject's CDR-SB score 701 is disclosed. In some embodiments, the method comprises identifying a subject as likely to benefit from an decreased CDR-SB score 702, and administering a therapeutically effective amount of an antibody, wherein the antibody binds to Gal3 703.

Some embodiments provided herein relate to a method of decreasing a subject's Clinical Dementia Rating Scale-Sum of Boxes (CDR-SB) score, the method comprising: identifying a subject as having Alzheimer's disease (AD), and Administering an antibody, wherein the antibody binds to Gal3.

FIG. 8 is a flow chart depicting some embodiments of a method of decreasing a subject's Clinical Dementia Rating Scale-Sum of Boxes (CDR-SB) score.

In some embodiments, a method of decreasing a subject's CDR-SB score 801 is disclosed. In some embodiments, the method comprises identifying a subject as having Alzheimer's disease 802, and administering a therapeutically effective amount of an antibody, wherein the antibody binds to Gal3 803.

Some embodiments provided herein relate to a method of decreasing a subject's Cognitive Drug Research battery score, the method comprising: identifying a subject as likely to benefit from an decreased Cognitive Drug Research battery score, and administering a therapeutically effective amount of an antibody, wherein the antibody binds to Gal3.

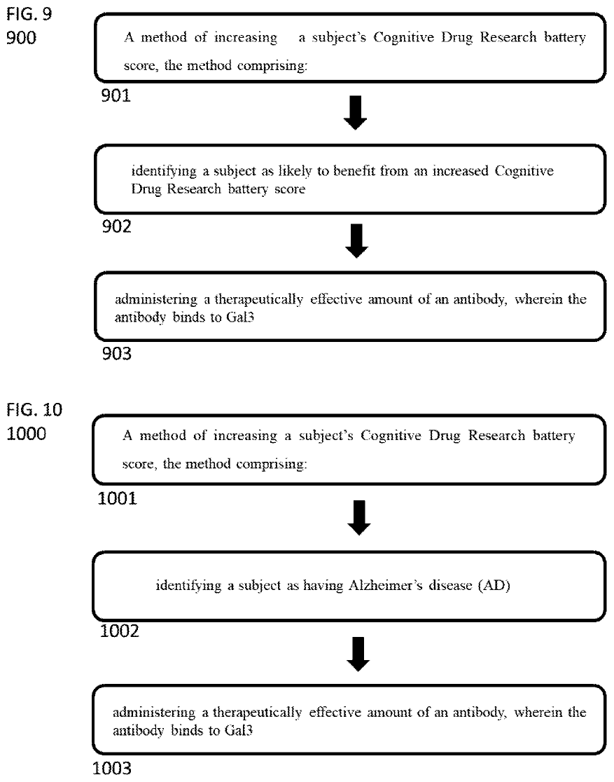

FIG. 9 is a flow chart depicting some embodiments of a method of increasing a subject's Cognitive Drug Research battery score.

In some embodiments, a method of increasing a subject's a subject's Cognitive Drug Research battery score 901 is disclosed. In some embodiments, the method comprises identifying a subject as likely to benefit from an increased Cognitive Drug Research battery score 902, and administering a therapeutically effective amount of an antibody, wherein the antibody binds to Gal3 903.

Some embodiments provided herein relate to a method of increasing a subject's Cognitive Drug Research battery score, the method comprising: identifying a subject as having Alzheimer's disease (AD), and administering a therapeutically effective amount of an antibody, wherein the antibody binds to Gal3.

FIG. 10 is a flow chart depicting some embodiments of a method of increasing a subject's Cognitive Drug Research battery score.

In some embodiments, a method of increasing a subject's Cognitive Drug Research battery score 1001 is disclosed. In some embodiments, the method comprises identifying a subject as having Alzheimer's disease 1002, and administering a therapeutically effective amount of an antibody, wherein the antibody binds to Gal3 1003.

Some embodiments provided herein relate to a method of increasing a subject's strength, locomotor functions, and/or balance, the method comprising: identifying a subject as likely to benefit from increased strength, locomotor functions, and/or balance, and administering a therapeutically effective amount of an antibody, wherein the antibody binds Gal3.

FIG. 11 is a flow chart depicting some embodiments of a method of increasing a subject's strength, locomotor functions, and/or balance.

In some embodiments, a method of increasing a subject's a strength, locomotor functions, and/or balance 1101 is disclosed. In some embodiments, the method comprises identifying a subject as likely to benefit from increased strength, locomotor functions, and/or balance 1102, and administering a therapeutically effective amount of an antibody, wherein the antibody binds to Gal3 1103.

Some embodiments provided herein relate to a method of increasing a subject's strength, locomotor functions, and/or balance, the method comprising: identifying a subject as having Alzheimer's disease (AD), and administering a therapeutically effective amount of an antibody, wherein the antibody binds Gal3.

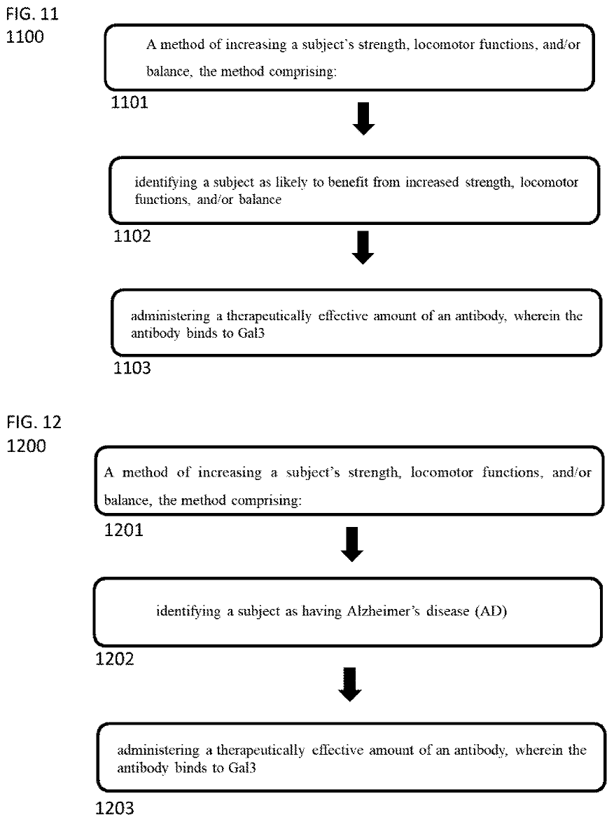

FIG. 12 is a flow chart depicting some embodiments of a method of increasing a subject's strength, locomotor functions, and/or balance.

In some embodiments, a method of increasing a subject's strength, locomotor functions, and/or balance 1201 is disclosed. In some embodiments, the method comprises identifying a subject as having Alzheimer's disease 1202, and administering a therapeutically effective amount of an antibody, wherein the antibody binds to Gal3 1203.

Some embodiments provided herein relate to a method of decreasing the frequency and/or severity of symptoms related to aging and/or aging related senescence in a subject in need thereof, the method comprising: identifying a subject as likely to benefit from decreased frequency and/or severity of symptoms related to aging and/or aging related senescence, and administering a therapeutically effective amount of an antibody, wherein the antibody binds Gal3.

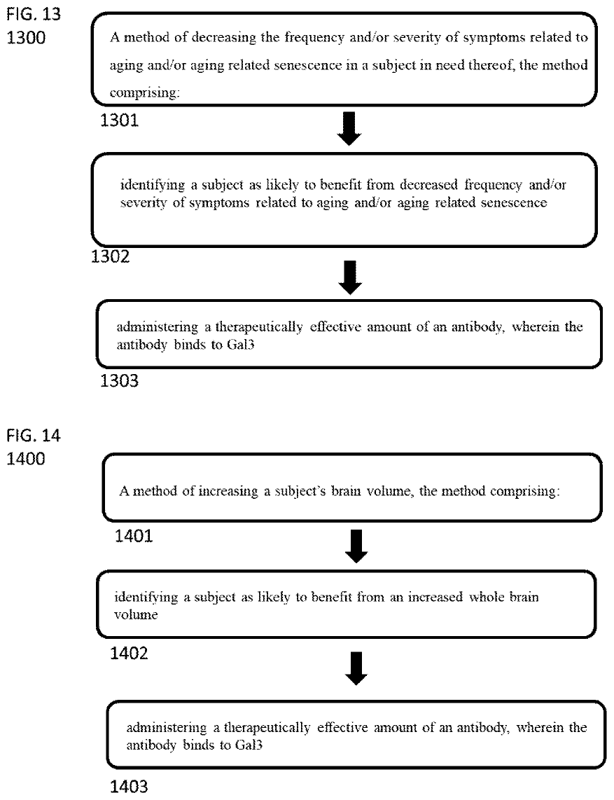

FIG. 13 is a flow chart depicting some embodiments of a method of decreasing the frequency and/or severity of symptoms related to aging and/or aging related senescence in a subject in need thereof.

In some embodiments, a method of decreasing the frequency and/or severity of symptoms related to aging and/or aging related senescence in a subject in need thereof 1301 is disclosed. In some embodiments, the method comprises identifying a subject as likely to benefit from decreasing the frequency and/or severity of symptoms related to aging and/or aging related senescence in a subject in need thereof 1302, and administering a therapeutically effective amount of an antibody, wherein the antibody binds to Gal3 1303.

Some embodiments provided herein relate to a method of increasing a subject's whole brain volume, the method comprising: identifying a subject as likely to benefit from an increased whole brain volume, and administering a therapeutically effective amount of an antibody, wherein the antibody binds to Gal3.

FIG. 14 is a flow chart depicting some embodiments of a method of increasing a subject's whole brain volume.

In some embodiments, a method of increasing a subject's whole brain volume 1401 is disclosed. In some embodiments, the method comprises identifying a subject as likely to benefit from increased whole brain volume 1402, and administering a therapeutically effective amount of an antibody, wherein the antibody binds to Gal3 1403.

Some embodiments provided herein relate to a method of decreasing brain atrophy in a subject, the method comprising: identifying a subject as likely to benefit from decreased brain atrophy, and administering a therapeutically effective amount of an antibody, wherein the antibody binds to Gal3.

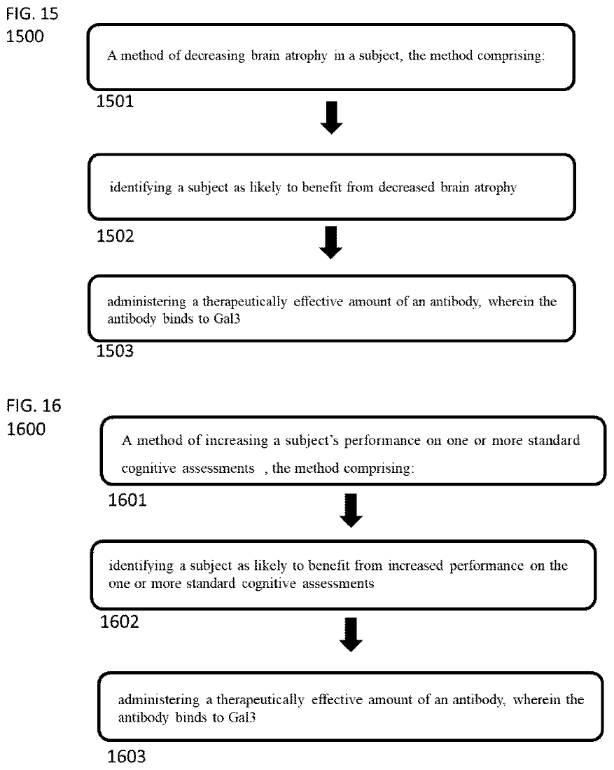

FIG. 15 is a flow chart depicting some embodiments of a method of decreasing brain atrophy in a subject.

In some embodiments, a method of decreasing brain atrophy in a subject 1501 is disclosed. In some embodiments, the method comprises identifying a subject as likely to benefit from decreased brain atrophy 1502, and administering a therapeutically effective amount of an antibody, wherein the antibody binds to Gal3 1503.

Some embodiments provided herein relate to a method of increasing a subject's performance on one or more standard cognitive assessments, the method comprising: identifying a subject as likely to benefit from increased performance on the one or more standard cognitive assessments, and administering a therapeutically effective amount of an antibody, wherein the antibody binds to Gal3.

FIG. 16 is a flow chart depicting some embodiments of a method of increasing a subject's performance on one or more standard cognitive assessments.

In some embodiments, a method of increasing a subject's performance on one or more standard cognitive assessments 1601 is disclosed. In some embodiments, the method comprises identifying a subject as likely to benefit from increased performance on one or more cognitive assessments 1602, and administering a therapeutically effective amount of an antibody, wherein the antibody binds to Gal3 1603.

Some embodiments provided herein relate to a method of increasing a subject's Mini Mental State Examination (MMSE) score, the method comprising: identifying a subject as having dementia, and administering a therapeutically effective amount of an antibody, wherein the antibody binds to Gal3.

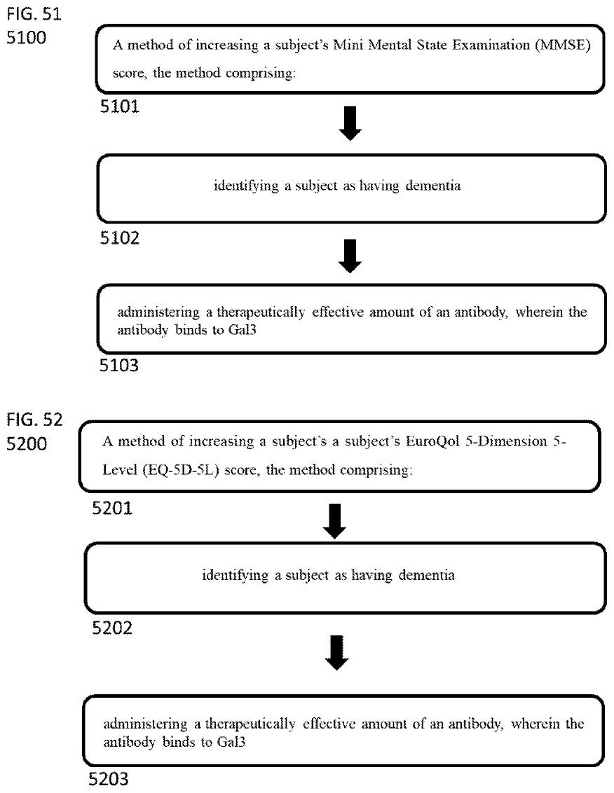

FIG. 51 is a flow chart depicting some embodiments of a method of increasing a subject's Mini Mental State Examination (MMSE) score.

In some embodiments, a method of increasing a subject's MMSE score 5101 is disclosed. In some embodiments, the method comprising: identifying a subject as having dementia 5102, and administering a therapeutically effective amount of an antibody, wherein the antibody binds to Gal3 5103.

Some embodiments provided herein relate to a method of increasing a subject's EuroQol 5-Dimension 5-Level (EQ-5D-5L), the method comprising: identifying a subject as having dementia, and administering a therapeutically effective amount of an antibody, wherein the antibody binds to Gal3.

FIG. 52 is a flow chart depicting some embodiments of a method of increasing a subject's EuroQol 5-Dimension 5-Level (EQ-5D-5L).

In some embodiments, a method of increasing a subject's EuroQol 5-Dimension 5-Level (EQ-5D-5L) 5201 is disclosed. In some embodiments, the method comprising: identifying a subject as having dementia 5202, and administering a therapeutically effective amount of an antibody, wherein the antibody binds to Gal3 5203.

Some embodiments provided herein relate to a method of decreasing a subject's a subject's Neuropsychiatric Inventory (NPI) score, the method comprising: identifying a subject as having dementia, and administering a therapeutically effective amount of an antibody, wherein the antibody binds to Gal3.

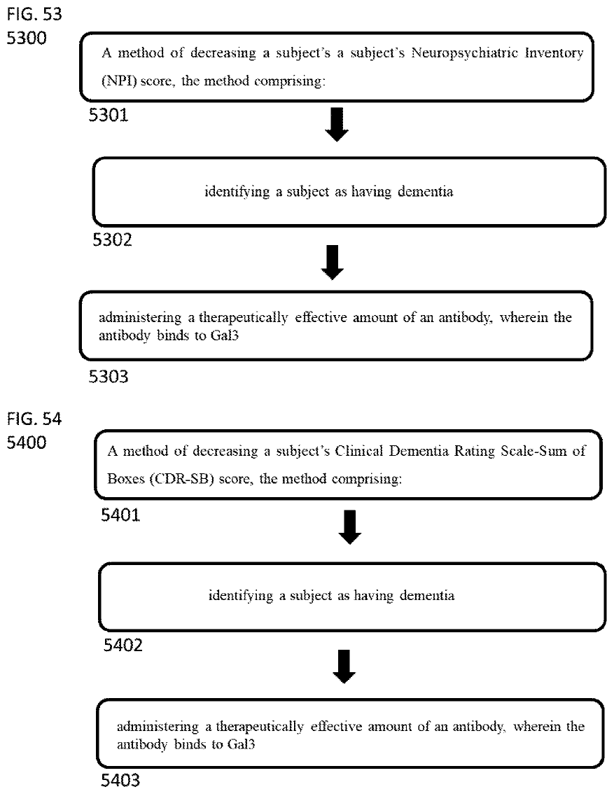

FIG. 53 is a flow chart depicting some embodiments of a method of decreasing a subject's NPI.

In some embodiments, a method of decreasing a subject's a subject's Neuropsychiatric Inventory (NPI) score 5301 is disclosed. In some embodiments, the method comprises identifying a subject as having dementia 5302, and administering a therapeutically effective amount of an antibody, wherein the antibody binds to Gal3 5303.

Some embodiments provided herein relate to a method of decreasing a subject's Clinical Dementia Rating Scale-Sum of Boxes (CDR-SB) score, the method comprising: identifying a subject as having dementia, and administering an antibody, wherein the antibody binds to Gal3.

FIG. 54 is a flow chart depicting some embodiments of a method of decreasing a subject's CDR-SB score.

In some embodiments, a method of decreasing a subject's a subject's CDR-SB score 5401 is disclosed. In some embodiments, the method comprises identifying a subject as having dementia 5402, and administering a therapeutically effective amount of an antibody, wherein the antibody binds to Gal3 5403.

Some embodiments provided herein relate to a method of increasing a subject's Cognitive Drug Research battery score, the method comprising: identifying a subject as having dementia, and administering a therapeutically effective amount of an antibody, wherein the antibody binds to Gal3.

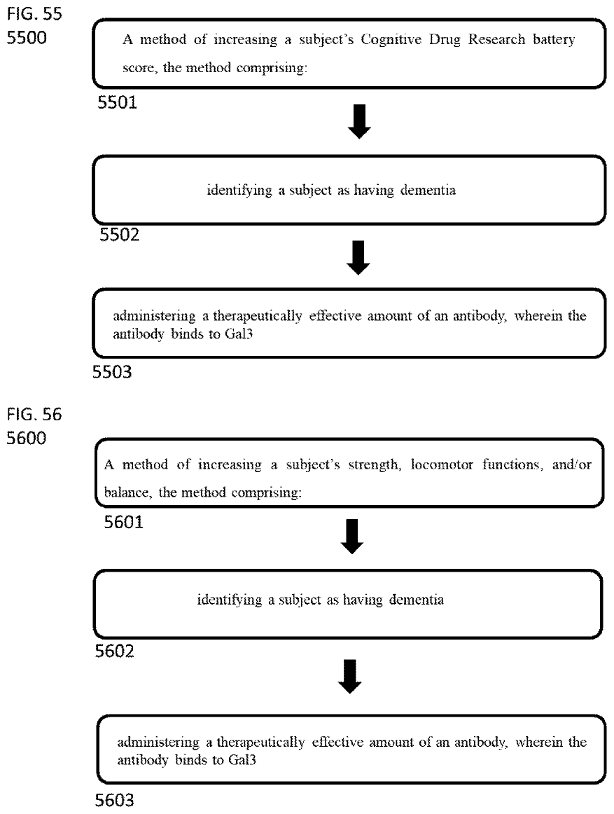

FIG. 55 is a flow chart depicting some embodiments of a method of increasing a subject's Cognitive Drug Research battery score.

In some embodiments, a method of increasing a subject's Cognitive Drug Research battery score 5501 is disclosed. In some embodiments, the method comprising: identifying a subject as having dementia 5502, and administering a therapeutically effective amount of an antibody, wherein the antibody binds to Gal3 5503.

Some embodiments provided herein relate to a method of increasing a subject's strength, locomotor functions, and/or balance, the method comprising: identifying a subject as having dementia, and administering a therapeutically effective amount of an antibody, wherein the antibody binds Gal3.

FIG. 56 is a flow chart depicting some embodiments of a method of increasing a subject's strength, locomotor functions, and/or balance.

In some embodiments, a method of increasing a subject's strength, locomotor functions, and/or balance 5601 is disclosed. In some embodiments, the method comprise identifying a subject as having dementia 5602, and administering a therapeutically effective amount of an antibody, wherein the antibody binds to Gal3 5603.

FIG. 82 is a chart depicting some embodiments of study design, treatment schema, and endpoints for parts 1b and 2a of a multi-center, double-blinded followed by an Open Label Extension (OLE) study.

In some embodiments, a method of decreasing a subject's Clinical Dementia Rating Scale-Sum of Boxes (CDR-SB) score is disclosed. In some embodiments, the method comprises identifying a subject as having dementia, and administering an antibody, wherein the antibody binds to Gal3. In some embodiments, the subjects are being administered anti-Gal3 antibody according to the protocols of the Examples herein, including Examples 1, 13, and 19. In some embodiments, the subject is administered 140 mg, 420 mg, or 1000 mg once weekly for 5 weeks. In some embodiments, the subject is administered 140 mg, 420, or 1000 mg once weekly for 5 weeks followed by administration of 4000 mg of the antibody once monthly for one or more months. In some embodiments, the subject is administered 140 mg, 420, or 1000 mg once weekly for 5 weeks followed by administration of 4000 mg of the antibody once monthly for one or more months decreases the subject's Clinical Dementia Rating Scale-Sum of Boxes (CDR-SB) score by up to about 0.5 points.

In some embodiments, a method of decreasing a subject's Clinical Dementia Rating Scale-Sum of Boxes (CDR-SB) score is disclosed. In some embodiments, the method comprises identifying a subject as likely to benefit from a decreased CDR-SB score, and administering 140, 420, or 1000 mg of an anti-Gal3 antibody once weekly for 5 weeks. In some embodiments, the antibody is administered 5 times in a month, once weekly at 140 mg. In some embodiments, the antibody is administered 5 times in a month, once weekly at 420 mg. In some embodiments, the antibody is administered 5 times in a month, once weekly at 1000 mg. In some embodiments, following the first month, the antibody is administered 5 times in a month, once weekly at 1000 mg. For example, in some embodiments, the antibody is administered 5 times in a month, once weekly at 140 mg, followed by administration 5 times in a month, once weekly at 1000 mg. In some embodiments, the antibody is administered 5 times in a month, once weekly at 420 mg, followed by administration 5 times in a month, once weekly at 1000 mg. In some embodiments, the antibody is administered 5 times in a month, once weekly at 1000 mg, followed by administration 5 times in a month, once weekly at 1000 mg. In some embodiments, following the first or second month, the antibody is administered once-monthly, about every 28 days at 4000 mg. For example, in some embodiments, the antibody is administered 5 times in a month, once weekly at 140 mg, followed by administration 5 times in a month, once weekly at 1000 mg, followed by administration once-monthly, about every 28 days at 4000 mg. In some embodiments, the antibody is administered 5 times in a month, once weekly at 420 mg, followed by administration 5 times in a month, once weekly at 1000 mg, followed by administration once-monthly, about every 28 days at 4000 mg. In some embodiments, the antibody is administered 5 times in a month, once weekly at 1000 mg, followed by administration 5 times in a month, once weekly at 1000 mg, followed by administration once-monthly, about every 28 days at 4000 mg. In some embodiments, the antibody is administered 5 times in a month, once weekly at 140 mg, followed by administration once-monthly, about every 28 days at 4000 mg. In some embodiments, the antibody is administered 5 times in a month, once weekly at 420 mg, followed by administration once-monthly, about every 28 days at 4000 mg. In some embodiments, the antibody is administered 5 times in a month, once weekly at 1000 mg, followed by administration once-monthly, about every 28 days at 4000 mg. In some embodiments, the treatment continues for about 1, 2, 3, 4, 5, 6, 7, 8, 9, 10, 11, 12, 12, 14, 15, 16, 17, 18, 19, 20, 21, 22, 23, or 24 months, or for a range that is defined by any two of the preceding values. For example, in some embodiments, the treatment continues for between about 1-24, 1-18, 1-12, 1-6, 1-3, 3-24, 3-18, 3-12, 3-6, 6-24, 6-18, 6-12, 12-24, 12-18, or 18-24, months. In some embodiments, the antibody is administered 5 times in a month, once weekly at 140 mg, followed by administration 5 times in a month, once weekly at 1000 mg, followed by administration once-monthly, about every 28 days at 4000 mg, for up to about 2 years. In some embodiments, the antibody is administered 5 times in a month, once weekly at 420 mg, followed by administration 5 times in a month, once weekly at 1000 mg, followed by administration once-monthly, about every 28 days at 4000 mg, for up to about 2 years. In some embodiments, the antibody is administered 5 times in a month, once weekly at 1000 mg, followed by administration 5 times in a month, once weekly at 1000 mg, followed by administration once-monthly, about every 28 days at 4000 mg, for up to about 2 years. In some embodiments, the antibody is administered 5 times in a month, once weekly at 140 mg, followed by administration once-monthly, about every 28 days at 4000 mg, for up to about 2 years. In some embodiments, the antibody is administered 5 times in a month, once weekly at 420 mg, followed by administration once-monthly, about every 28 days at 4000 mg, for up to about 2 years. In some embodiments, the antibody is administered 5 times in a month, once weekly at 1000 mg, followed by administration once-monthly, about every 28 days at 4000 mg, for up to about 2 years. In some embodiments, the treatment continues for the life of the patient. In some embodiments, the antibody is administered 5 times in a month, once weekly at 140 mg, followed by administration 5 times in a month, once weekly at 1000 mg, followed by administration once-monthly, about every 28 days at 4000 mg, for the life of the patient. In some embodiments, the antibody is administered 5 times in a month, once weekly at 420 mg, followed by administration 5 times in a month, once weekly at 1000 mg, followed by administration once-monthly, about every 28 days at 4000 mg, for the life of the patient. In some embodiments, the antibody is administered 5 times in a month, once weekly at 1000 mg, followed by administration 5 times in a month, once weekly at 1000 mg, followed by administration once-monthly, about every 28 days at 4000 mg, for the life of the patient. In some embodiments, the antibody is administered 5 times in a month, once weekly at 140 mg, followed by administration once-monthly, about every 28 days at 4000 mg, for the life of the patient. In some embodiments, the antibody is administered 5 times in a month, once weekly at 420 mg, followed by administration once-monthly, about every 28 days at 4000 mg, for the life of the patient. In some embodiments, the antibody is administered 5 times in a month, once weekly at 1000 mg, followed by administration once-monthly, about every 28 days at 4000 mg, for the life of the patient. In some embodiments, administration of the antibody results in a decrease in the subject's CDR-SB score. In some embodiments, the subject's CDR-SB score decreases by 0.1, 0.2, 0.3, 0.4, 0.5, 0.6, 0.7, 0.8, 0.9, 1, 1.5, 2, or 2.5 points, or by a range that is defined by any two of the preceding values. For example, in some embodiments, the subject's CDR-SB score is decreased by about 0.1-2.5, 0.1-2, 0.1-1.5, 0.1-1, 0.1-0.5, 0.5-2.5, 0.5-2, 0.5-1.5, 0.5-1, 1-2.5, 1-2, 1-1.5, 1.5-2.5, 1.5-2, or 2-2.5, points following one or more administrations of the antibody. In some embodiments, the antibody is administered by IV infusion. In some embodiments, the antibody is administered as a therapeutic formulation. In some embodiments, the antibody is administered by IV as therapeutic formulation comprising 20 mg/mL (in some embodiments, the 20 mg/ml can be diluted to 8 mg/ml in a total of 500 mL prior to I. V. administration) antibody, 20 mM L-Histidine, 5 mM Methionine, 100 mM NaCl, 0.02% PS80, and a pH of 5.8, over the course of 1-hour for a total administered dose of 4000 mg. In some embodiments, the antibody is administered subcutaneously. In some embodiments, the antibody is TB006. In some embodiments, the dose administered is chosen to achieve an improved CDR-SB score. In some embodiments, the dose administered is chosen to achieve a decrease in the subject's CDR-SB score of between 0.1-2.5 points.

In some embodiments, a method of decreasing a subject's Clinical Dementia Rating Scale-Sum of Boxes (CDR-SB) score is disclosed. In some embodiments, the method comprises identifying a subject as likely to benefit from a decreased CDR-SB score, and administering 140, 420, or 1000 mg of TB006 antibody once weekly for 5 weeks. In some embodiments, TB006 is administered 5 times in a month, once weekly at 140 mg. In some embodiments, TB006 is administered 5 times in a month, once weekly at 420 mg. In some embodiments, TB006 is administered 5 times in a month, once weekly at 1000 mg. In some embodiments, following the first month, TB006 is administered 5 times in a month, once weekly at 1000 mg. For example, in some embodiments, TB006 is administered 5 times in a month, once weekly at 140 mg, followed by administration 5 times in a month, once weekly at 1000 mg. In some embodiments, TB006 is administered 5 times in a month, once weekly at 420 mg, followed by administration 5 times in a month, once weekly at 1000 mg. In some embodiments, TB006 is administered 5 times in a month, once weekly at 1000 mg, followed by administration 5 times in a month, once weekly at 1000 mg. In some embodiments, following the first or second month, TB006 is administered once-monthly, about every 28 days at 4000 mg. For example, in some embodiments, TB006 is administered 5 times in a month, once weekly at 140 mg, followed by administration 5 times in a month, once weekly at 1000 mg, followed by administration once-monthly, about every 28 days at 4000 mg. In some embodiments, TB006 is administered 5 times in a month, once weekly at 420 mg, followed by administration 5 times in a month, once weekly at 1000 mg, followed by administration once-monthly, about every 28 days at 4000 mg. In some embodiments, TB006 is administered 5 times in a month, once weekly at 1000 mg, followed by administration 5 times in a month, once weekly at 1000 mg, followed by administration once-monthly, about every 28 days at 4000 mg. In some embodiments, TB006 is administered 5 times in a month, once weekly at 140 mg, followed by administration once-monthly, about every 28 days at 4000 mg. In some embodiments, TB006 is administered 5 times in a month, once weekly at 420 mg, followed by administration once-monthly, about every 28 days at 4000 mg. In some embodiments, TB006 is administered 5 times in a month, once weekly at 1000 mg, followed by administration once-monthly, about every 28 days at 4000 mg. In some embodiments, the treatment continues for about 1, 2, 3, 4, 5, 6, 7, 8, 9, 10, 11, 12, 12, 14, 15, 16, 17, 18, 19, 20, 21, 22, 23, or 24 months, or for a range that is defined by any two of the preceding values. For example, in some embodiments, the treatment continues for between about 1-24, 1-18, 1-12, 1-6, 1-3, 3-24, 3-18, 3-12, 3-6, 6-24, 6-18, 6-12, 12-24, 12-18, or 18-24, months. In some embodiments, TB006 is administered 5 times in a month, once weekly at 140 mg, followed by administration 5 times in a month, once weekly at 1000 mg, followed by administration once-monthly, about every 28 days at 4000 mg, for up to about 2 years. In some embodiments, TB006 is administered 5 times in a month, once weekly at 420 mg, followed by administration 5 times in a month, once weekly at 1000 mg, followed by administration once-monthly, about every 28 days at 4000 mg, for up to about 2 years. In some embodiments, TB006 is administered 5 times in a month, once weekly at 1000 mg, followed by administration 5 times in a month, once weekly at 1000 mg, followed by administration once-monthly, about every 28 days at 4000 mg, for up to about 2 years. In some embodiments, TB006 is administered 5 times in a month, once weekly at 140 mg, followed by administration once-monthly, about every 28 days at 4000 mg, for up to about 2 years. In some embodiments, TB006 is administered 5 times in a month, once weekly at 420 mg, followed by administration once-monthly, about every 28 days at 4000 mg, for up to about 2 years. In some embodiments, TB006 is administered 5 times in a month, once weekly at 1000 mg, followed by administration once-monthly, about every 28 days at 4000 mg, for up to about 2 years. In some embodiments, the treatment continues for the life of the patient. In some embodiments, TB006 is administered 5 times in a month, once weekly at 140 mg, followed by administration 5 times in a month, once weekly at 1000 mg, followed by administration once-monthly, about every 28 days at 4000 mg, for the life of the patient. In some embodiments, TB006 is administered 5 times in a month, once weekly at 420 mg, followed by administration 5 times in a month, once weekly at 1000 mg, followed by administration once-monthly, about every 28 days at 4000 mg, for the life of the patient. In some embodiments, TB006 is administered 5 times in a month, once weekly at 1000 mg, followed by administration 5 times in a month, once weekly at 1000 mg, followed by administration once-monthly, about every 28 days at 4000 mg, for the life of the patient. In some embodiments, the antibody is administered 5 times in a month, once weekly at 140 mg, followed by administration once-monthly, about every 28 days at 4000 mg, for the life of the patient. In some embodiments, TB006 is administered 5 times in a month, once weekly at 420 mg, followed by administration once-monthly, about every 28 days at 4000 mg, for the life of the patient. In some embodiments, TB006 is administered 5 times in a month, once weekly at 1000 mg, followed by administration once-monthly, about every 28 days at 4000 mg, for the life of the patient. In some embodiments, administration of the antibody results in a decrease in the subject's CDR-SB score. In some embodiments, the subject's CDR-SB score decreases by 0.1, 0.2, 0.3, 0.4, 0.5, 0.6, 0.7, 0.8, 0.9, 1, 1.5, 2, or 2.5 points, or by a range that is defined by any two of the preceding values. For example, in some embodiments, the subject's CDR-SB score is decreased by about 0.1-2.5, 0.1-2, 0.1-1.5, 0.1-1, 0.1-0.5, 0.5-2.5, 0.5-2, 0.5-1.5, 0.5-1, 1-2.5, 1-2, 1-1.5, 1.5-2.5, 1.5-2, or 2-2.5, points following one or more administrations of TB006. In some embodiments, TB006 is administered by IV infusion. In some embodiments, TB006 is administered as a therapeutic formulation. In some embodiments, TB006 is administered by IV as therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, over the course of 1-hour for a total administered dose of 4000 mg. In some embodiments, TB006 is administered subcutaneously. In some embodiments, the dose of antibody administered is up to 5000 mg. In some embodiments, the dose of antibody administered is between 70 and 4000 mg. For example, in some embodiments, the dose of antibody administered is 70, 100, 140, 250, 420, 500, 600, 700, 750, 800, 900, 1000, 1250, 1500, 1750, 2000, 2250, 2500, 2750, 3000, 3250, 3500, 3750, 4000, 4250, 4500, 4750, or 5000 mg, or a range that is defined by any two of the preceding values. In some embodiments, the dose administered is chosen to achieve an improved CDR-SB score. In some embodiments, the dose administered is chosen to achieve a decrease in the subject's CDR-SB score of between 0.1-2.5 points.

In some embodiments, a method of decreasing a subject's Clinical Dementia Rating Scale-Sum of Boxes (CDR-SB) score is disclosed. In some embodiments, the method comprises identifying a subject as likely to benefit from a decreased CDR-SB score, and administering a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 140 mg once weekly for 5 weeks. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 420 mg once weekly for 5 weeks. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly 5 times in a month. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly 5 times in a month. In some embodiments, following the first month, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly is administered 5 times in a month. For example, in some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 140 mg once weekly is administered 5 times in a month, followed by administration 5 times in a month, once weekly at 1000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 420 mg once weekly is administered 5 times in a month, followed by administration 5 times in a month, once weekly at 1000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly is administered 5 times in a month, followed by administration 5 times in a month, once weekly at 1000 mg. In some embodiments, following the first or second month, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 4000 mg is administered once-monthly, about every 28 days. For example, in some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour for a total administered dose of 140 mg 5 times in a month, followed by administration 5 times in a month, once weekly at 1000 mg, followed by administration once-monthly, about every 28 days at 4000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methioninc, 100 mM NaCl, 0.02% PS80, and a pH of 5.8, intravenously over the course of 1-hour for a total administered dose of 420 mg once weekly is administered 5 times in a month, once weekly, followed by administration 5 times in a month, once weekly at 1000 mg, followed by administration once-monthly, about every 28 days at 4000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly is administered 5 times in a month, once weekly, followed by administration 5 times in a month, once weekly at 1000 mg, followed by administration once-monthly, about every 28 days at 4000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 140 mg once weekly is administered 5 times in a month, once weekly, followed by administration once-monthly, about every 28 days at 4000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 420 mg is administered 5 times in a month, once weekly, followed by administration once-monthly, about every 28 days at 4000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly is administered 5 times in a month, once weekly, followed by administration once-monthly, about every 28 days at 4000 mg. In some embodiments, the treatment continues for about 1, 2, 3, 4, 5, 6, 7, 8, 9, 10, 11, 12, 12, 14, 15, 16, 17, 18, 19, 20, 21, 22, 23, or 24 months, or for a range that is defined by any two of the preceding values. For example, in some embodiments, the treatment continues for between about 1-24, 1-18, 1-12, 1-6, 1-3, 3-24, 3-18, 3-12, 3-6, 6-24, 6-18, 6-12, 12-24, 12-18, or 18-24, months. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour for a total administered dose of 140 mg, once weekly, 5 times in a month, followed by administration 5 times in a month, once weekly at 1000 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour for a total administered dose of 420 mg once weekly 5 times in a month, followed by administration 5 times in a month, once weekly at 1000 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 1000 mg, followed by administration 5 times in a month, once weekly at 1000 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 140 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 420 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methioninc, 100 mM NaCl, 0.02% PS80, and a pH of 5.8, is administered intravenously over the course of 1-hour, 5 times in a month, once weekly, for a total administered dose of 1000 mg, followed by administration once-monthly, about every 28 days at 4000 mg, for up to about 2 years. In some embodiments, the treatment continues for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 140 mg, followed by administration 5 times in a month, once weekly at 1000 mg, followed by administration once-monthly, about every 28 days at 4000 mg, for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 420 mg, followed by administration 5 times in a month, once weekly at 1000 mg, followed by administration once-monthly, about every 28 days at 4000 mg, for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 1000 mg, followed by administration 5 times in a month, once weekly at 1000 mg, followed by administration once-monthly, about every 28 days at 4000 mg, for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 140 mg, followed by administration once-monthly, about every 28 days at 4000 mg, for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 420 mg, followed by administration once-monthly, about every 28 days at 4000 mg, for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 1000 mg, followed by administration once-monthly, about every 28 days at 4000 mg, for the life of the patient. In some embodiments, administration of a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, results in an decrease in the subject's CDR-SB score. In some embodiments, administration of the antibody results in a decrease in the subject's CDR-SB score. In some embodiments, the subject's CDR-SB score decreases by 0.1, 0.2, 0.3, 0.4, 0.5, 0.6, 0.7, 0.8, 0.9, 1, 1.5, 2, or 2.5 points, or by a range that is defined by any two of the preceding values. For example, in some embodiments, the subject's CDR-SB score is decreased by about 0.1-2.5, 0.1-2, 0.1-1.5, 0.1-1, 0.1-0.5, 0.5-2.5, 0.5-2, 0.5-1.5, 0.5-1, 1-2.5, 1-2, 1-1.5, 1.5-2.5, 1.5-2, or 2-2.5, points following one or more administrations administration of a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8. In some embodiments, the dose of antibody administered is between 70 and 4000 mg. For example, in some embodiments, the dose of antibody administered is 70, 100, 140, 250, 420, 500, 600, 700, 750, 800, 900, 1000, 1250, 1500, 1750, 2000, 2250, 2500, 2750, 3000, 3250, 3500, 3750, 4000, 4250, 4500, 4750, or 5000 mg, or a range that is defined by any two of the preceding values. In some embodiments, the dose administered is chosen to achieve an improved CDR-SB score. In some embodiments, the dose administered is chosen to achieve an decrease in the subject's CDR-SB score of between 0.1-2.5 points.

In some embodiments, a method of increasing a subject's Mini Mental State Examination (MMSE) score and decreasing the subject's Clinical Dementia Rating Scale-Sum of Boxes (CDR-SB) score is disclosed. In some embodiments, the method comprises identifying a subject as likely to benefit from an increased MMSE score and decreased CDR-SB score, and administering 140, 420, or 1000 mg of an anti-Gal3 antibody once weekly for 5 weeks. In some embodiments, the antibody is administered 5 times in a month, once weekly at 140 mg. In some embodiments, the antibody is administered 5 times in a month, once weekly at 420 mg. In some embodiments, the antibody is administered 5 times in a month, once weekly at 1000 mg. In some embodiments, following the first month, the antibody is administered 5 times in a month, once weekly at 1000 mg. For example, in some embodiments, the antibody is administered 5 times in a month, once weekly at 140 mg, followed by administration 5 times in a month, once weekly at 1000 mg. In some embodiments, the antibody is administered 5 times in a month, once weekly at 420 mg, followed by administration 5 times in a month, once weekly at 1000 mg. In some embodiments, the antibody is administered 5 times in a month, once weekly at 1000 mg, followed by administration 5 times in a month, once weekly at 1000 mg. In some embodiments, following the first or second month, the antibody is administered once-monthly, about every 28 days at 4000 mg. For example, in some embodiments, the antibody is administered 5 times in a month, once weekly at 140 mg, followed by administration 5 times in a month, once weekly at 1000 mg, followed by administration once-monthly, about every 28 days at 4000 mg. In some embodiments, the antibody is administered 5 times in a month, once weekly at 420 mg, followed by administration 5 times in a month, once weekly at 1000 mg, followed by administration once-monthly, about every 28 days at 4000 mg. In some embodiments, the antibody is administered 5 times in a month, once weekly at 1000 mg, followed by administration 5 times in a month, once weekly at 1000 mg, followed by administration once-monthly, about every 28 days at 4000 mg. In some embodiments, the antibody is administered 5 times in a month, once weekly at 140 mg, followed by administration once-monthly, about every 28 days at 4000 mg. In some embodiments, the antibody is administered 5 times in a month, once weekly at 420 mg, followed by administration once-monthly, about every 28 days at 4000 mg. In some embodiments, the antibody is administered 5 times in a month, once weekly at 1000 mg, followed by administration once-monthly, about every 28 days at 4000 mg. In some embodiments, the treatment continues for about 1, 2, 3, 4, 5, 6, 7, 8, 9, 10, 11, 12, 12, 14, 15, 16, 17, 18, 19, 20, 21, 22, 23, or 24 months, or for a range that is defined by any two of the preceding values. For example, in some embodiments, the treatment continues for between about 1-24, 1-18, 1-12, 1-6, 1-3, 3-24, 3-18, 3-12, 3-6, 6-24, 6-18, 6-12, 12-24, 12-18, or 18-24, months. In some embodiments, the antibody is administered 5 times in a month, once weekly at 140 mg, followed by administration 5 times in a month, once weekly at 1000 mg, followed by administration once-monthly, about every 28 days at 4000 mg, for up to about 2 years. In some embodiments, the antibody is administered 5 times in a month, once weekly at 420 mg, followed by administration 5 times in a month, once weekly at 1000 mg, followed by administration once-monthly, about every 28 days at 4000 mg, for up to about 2 years. In some embodiments, the antibody is administered 5 times in a month, once weekly at 1000 mg, followed by administration 5 times in a month, once weekly at 1000 mg, followed by administration once-monthly, about every 28 days at 4000 mg, for up to about 2 years. In some embodiments, the antibody is administered 5 times in a month, once weekly at 140 mg, followed by administration once-monthly, about every 28 days at 4000 mg, for up to about 2 years. In some embodiments, the antibody is administered 5 times in a month, once weekly at 420 mg, followed by administration once-monthly, about every 28 days at 4000 mg, for up to about 2 years. In some embodiments, the antibody is administered 5 times in a month, once weekly at 1000 mg, followed by administration once-monthly, about every 28 days at 4000 mg, for up to about 2 years. In some embodiments, the treatment continues for the life of the patient. In some embodiments, the antibody is administered 5 times in a month, once weekly at 140 mg, followed by administration 5 times in a month, once weekly at 1000 mg, followed by administration once-monthly, about every 28 days at 4000 mg, for the life of the patient. In some embodiments, the antibody is administered 5 times in a month, once weekly at 420 mg, followed by administration 5 times in a month, once weekly at 1000 mg, followed by administration once-monthly, about every 28 days at 4000 mg, for the life of the patient. In some embodiments, the antibody is administered 5 times in a month, once weekly at 1000 mg, followed by administration 5 times in a month, once weekly at 1000 mg, followed by administration once-monthly, about every 28 days at 4000 mg, for the life of the patient. In some embodiments, the antibody is administered 5 times in a month, once weekly at 140 mg, followed by administration once-monthly, about every 28 days at 4000 mg, for the life of the patient. In some embodiments, the antibody is administered 5 times in a month, once weekly at 420 mg, followed by administration once-monthly, about every 28 days at 4000 mg, for the life of the patient. In some embodiments, the antibody is administered 5 times in a month, once weekly at 1000 mg, followed by administration once-monthly, about every 28 days at 4000 mg, for the life of the patient. In some embodiments, administration of the antibody results in an increase in the subject's MMSE score and/or a decrease in the subject's CDR-SB score. In some embodiments, the subject's MMSE score increases by 1, 2, 3, 4, 5, 6, 7, 8, 9, 10, 11, 12, 12, 14, or 15, points, or by a range that is defined by any two of the preceding values. For example, in some embodiments, the subject's MMSE score is increased by about 1-15, 1-10, 1-7, 1-5, 1-3, 3-15, 3-10, 3-7, 3-5, 5-15, 5-10, 5-7, 7-15 7-10, or 10-15, points following one or more administrations of the antibody. In some embodiments, the subject's CDR-SB score decreases by 0.1, 0.2, 0.3, 0.4, 0.5, 0.6, 0.7, 0.8, 0.9, 1, 1.5, 2, or 2.5 points, or by a range that is defined by any two of the preceding values. For example, in some embodiments, the subject's CDR-SB score is decreased by about 0.1-2.5, 0.1-2, 0.1-1.5, 0.1-1, 0.1-0.5, 0.5-2.5, 0.5-2, 0.5-1.5, 0.5-1, 1-2.5, 1-2, 1-1.5, 1.5-2.5, 1.5-2, or 2-2.5, following one or more administrations of the antibody. In some embodiments, the antibody is administered by IV infusion. In some embodiments, the antibody is administered as a therapeutic formulation. In some embodiments, the antibody is administered by IV as therapeutic formulation comprising 20 mg/mL (in some embodiments, the 20 mg/ml can be diluted to 8 mg/ml in a total of 500 mL prior to I. V. administration) antibody, 20 mM L-Histidine, 5 mM Methionine, 100 mM NaCl, 0.02% PS80, and a pH of 5.8, over the course of 1-hour for a total administered dose of 4000 mg. In some embodiments, the antibody is administered subcutaneously. In some embodiments, the antibody is TB006. In some embodiments, the dose administered is chosen to achieve an improved MMSE and/or CDR-SB score. In some embodiments, the dose administered is chosen to achieve an increase in the subject's MMSE score of between 1-15 points and a decrease in the subject's CDR-SB score of between 0.1 and 2.5 points.

In some embodiments, a method of increasing a subject's Mini Mental State Examination (MMSE) score and decreasing the subject's Clinical Dementia Rating Scale-Sum of Boxes (CDR-SB) score is disclosed. In some embodiments, the method comprises identifying a subject as likely to benefit from an increased MMSE score and decreased CDR-SB score, and administering 140, 420, or 1000 mg of TB006 antibody once weekly for 5 weeks. In some embodiments, TB006 is administered 5 times in a month, once weekly at 140 mg. In some embodiments, TB006 is administered 5 times in a month, once weekly at 420 mg. In some embodiments, TB006 is administered 5 times in a month, once weekly at 1000 mg. In some embodiments, following the first month, TB006 is administered 5 times in a month, once weekly at 1000 mg. For example, in some embodiments, TB006 is administered 5 times in a month, once weekly at 140 mg, followed by administration 5 times in a month, once weekly at 1000 mg. In some embodiments, TB006 is administered 5 times in a month, once weekly at 420 mg, followed by administration 5 times in a month, once weekly at 1000 mg. In some embodiments, TB006 is administered 5 times in a month, once weekly at 1000 mg, followed by administration 5 times in a month, once weekly at 1000 mg. In some embodiments, following the first or second month, TB006 is administered once-monthly, about every 28 days at 4000 mg. For example, in some embodiments, TB006 is administered 5 times in a month, once weekly at 140 mg, followed by administration 5 times in a month, once weekly at 1000 mg, followed by administration once-monthly, about every 28 days at 4000 mg. In some embodiments, TB006 is administered 5 times in a month, once weekly at 420 mg, followed by administration 5 times in a month, once weekly at 1000 mg, followed by administration once-monthly, about every 28 days at 4000 mg. In some embodiments, TB006 is administered 5 times in a month, once weekly at 1000 mg, followed by administration 5 times in a month, once weekly at 1000 mg, followed by administration once-monthly, about every 28 days at 4000 mg. In some embodiments, TB006 is administered 5 times in a month, once weekly at 140 mg, followed by administration once-monthly, about every 28 days at 4000 mg. In some embodiments, TB006 is administered 5 times in a month, once weekly at 420 mg, followed by administration once-monthly, about every 28 days at 4000 mg. In some embodiments, TB006 is administered 5 times in a month, once weekly at 1000 mg, followed by administration once-monthly, about every 28 days at 4000 mg. In some embodiments, the treatment continues for about 1, 2, 3, 4, 5, 6, 7, 8, 9, 10, 11, 12, 12, 14, 15, 16, 17, 18, 19, 20, 21, 22, 23, or 24 months, or for a range that is defined by any two of the preceding values. For example, in some embodiments, the treatment continues for between about 1-24, 1-18, 1-12, 1-6, 1-3, 3-24, 3-18, 3-12, 3-6, 6-24, 6-18, 6-12, 12-24, 12-18, or 18-24, months. In some embodiments, TB006 is administered 5 times in a month, once weekly at 140 mg, followed by administration 5 times in a month, once weekly at 1000 mg, followed by administration once-monthly, about every 28 days at 4000 mg, for up to about 2 years. In some embodiments, TB006 is administered 5 times in a month, once weekly at 420 mg, followed by administration 5 times in a month, once weekly at 1000 mg, followed by administration once-monthly, about every 28 days at 4000 mg, for up to about 2 years. In some embodiments, TB006 is administered 5 times in a month, once weekly at 1000 mg, followed by administration 5 times in a month, once weekly at 1000 mg, followed by administration once-monthly, about every 28 days at 4000 mg, for up to about 2 years. In some embodiments, TB006 is administered 5 times in a month, once weekly at 140 mg, followed by administration once-monthly, about every 28 days at 4000 mg, for up to about 2 years. In some embodiments, TB006 is administered 5 times in a month, once weekly at 420 mg, followed by administration once-monthly, about every 28 days at 4000 mg, for up to about 2 years. In some embodiments, TB006 is administered 5 times in a month, once weekly at 1000 mg, followed by administration once-monthly, about every 28 days at 4000 mg, for up to about 2 years. In some embodiments, the treatment continues for the life of the patient. In some embodiments, TB006 is administered 5 times in a month, once weekly at 140 mg, followed by administration 5 times in a month, once weekly at 1000 mg, followed by administration once-monthly, about every 28 days at 4000 mg, for the life of the patient. In some embodiments, TB006 is administered 5 times in a month, once weekly at 420 mg, followed by administration 5 times in a month, once weekly at 1000 mg, followed by administration once-monthly, about every 28 days at 4000 mg, for the life of the patient. In some embodiments, TB006 is administered 5 times in a month, once weekly at 1000 mg, followed by administration 5 times in a month, once weekly at 1000 mg, followed by administration once-monthly, about every 28 days at 4000 mg, for the life of the patient. In some embodiments, the antibody is administered 5 times in a month, once weekly at 140 mg, followed by administration once-monthly, about every 28 days at 4000 mg, for the life of the patient. In some embodiments, TB006 is administered 5 times in a month, once weekly at 420 mg, followed by administration once-monthly, about every 28 days at 4000 mg, for the life of the patient. In some embodiments, TB006 is administered 5 times in a month, once weekly at 1000 mg, followed by administration once-monthly, about every 28 days at 4000 mg, for the life of the patient. In some embodiments, administration of TB006 results in an increase in the subject's MMSE score and/or a decrease in the subject's CDR-SB score. In some embodiments, the subject's MMSE score increases by 1, 2, 3, 4, 5, 6, 7, 8, 9, 10, 11, 12, 12, 14, or 15, points, or by a range that is defined by any two of the preceding values. For example, in some embodiments, the subject's MMSE score is increased by about 1-15, 1-10, 1-7, 1-5, 1-3, 3-15, 3-10, 3-7, 3-5, 5-15, 5-10, 5-7, 7-15 7-10, or 10-15, points following one or more administrations of TB006. In some embodiments, TB006 is administered by IV infusion. In some embodiments, the subject's CDR-SB score decreases by 0.1, 0.2, 0.3, 0.4, 0.5, 0.6, 0.7, 0.8, 0.9, 1, 1.5, 2, or 2.5 points, or by a range that is defined by any two of the preceding values. For example, in some embodiments, the subject's CDR-SB score is decreased by about 0.1-2.5, 0.1-2, 0.1-1.5, 0.1-1, 0.1-0.5, 0.5-2.5, 0.5-2, 0.5-1.5, 0.5-1, 1-2.5, 1-2, 1-1.5, 1.5-2.5, 1.5-2, or 2-2.5, following one or more administrations of TB006. In some embodiments, TB006 is administered as a therapeutic formulation. In some embodiments, TB006 is administered by IV as therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, over the course of 1-hour for a total administered dose of 4000 mg. In some embodiments, TB006 is administered subcutaneously. In some embodiments, the dose of antibody administered is up to 5000 mg. In some embodiments, the dose of antibody administered is between 70 and 4000 mg. For example, in some embodiments, the dose of antibody administered is 70, 100, 140, 250, 420, 500, 600, 700, 750, 800, 900, 1000, 1250, 1500, 1750, 2000, 2250, 2500, 2750, 3000, 3250, 3500, 3750, 4000, 4250, 4500, 4750, or 5000 mg, or a range that is defined by any two of the preceding values. In some embodiments, the dose administered is chosen to achieve an improved MMSE and/or CDR-SB score. In some embodiments, the dose administered is chosen to achieve an increase in the subject's MMSE score of between 1-15 points and a decrease in the subject's CDR-SB score of between 0.1 and 2.5 points.

In some embodiments, a method of increasing a subject's Mini Mental State Examination (MMSE) score and decreasing the subject's Clinical Dementia Rating Scale-Sum of Boxes (CDR-SB) score is disclosed. In some embodiments, the method comprises identifying a subject as likely to benefit from an increased MMSE score and decreased CDR-SB score, and administering a therapeutic formulation comprising 20 mg/ml (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 140 mg once weekly for 5 weeks. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 420 mg once weekly for 5 weeks. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly 5 times in a month. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly 5 times in a month. In some embodiments, following the first month, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly is administered 5 times in a month. For example, in some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 140 mg once weekly is administered 5 times in a month, followed by administration 5 times in a month, once weekly at 1000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 420 mg once weekly is administered 5 times in a month, followed by administration 5 times in a month, once weekly at 1000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly is administered 5 times in a month, followed by administration 5 times in a month, once weekly at 1000 mg. In some embodiments, following the first or second month, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 4000 mg is administered once-monthly, about every 28 days. For example, in some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour for a total administered dose of 140 mg 5 times in a month, followed by administration 5 times in a month, once weekly at 1000 mg, followed by administration once-monthly, about every 28 days at 4000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 420 mg once weekly is administered 5 times in a month, once weekly, followed by administration 5 times in a month, once weekly at 1000 mg, followed by administration once-monthly, about every 28 days at 4000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly is administered 5 times in a month, once weekly, followed by administration 5 times in a month, once weekly at 1000 mg, followed by administration once-monthly, about every 28 days at 4000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 140 mg once weekly is administered 5 times in a month, once weekly, followed by administration once-monthly, about every 28 days at 4000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 420 mg is administered 5 times in a month, once weekly, followed by administration once-monthly, about every 28 days at 4000 mg. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, intravenously over the course of 1-hour for a total administered dose of 1000 mg once weekly is administered 5 times in a month, once weekly, followed by administration once-monthly, about every 28 days at 4000 mg. In some embodiments, the treatment continues for about 1, 2, 3, 4, 5, 6, 7, 8, 9, 10, 11, 12, 12, 14, 15, 16, 17, 18, 19, 20, 21, 22, 23, or 24 months, or for a range that is defined by any two of the preceding values. For example, in some embodiments, the treatment continues for between about 1-24, 1-18, 1-12, 1-6, 1-3, 3-24, 3-18, 3-12, 3-6, 6-24, 6-18, 6-12, 12-24, 12-18, or 18-24, months. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour for a total administered dose of 140 mg, once weekly, 5 times in a month, followed by administration 5 times in a month, once weekly at 1000 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour for a total administered dose of 420 mg once weekly 5 times in a month, followed by administration 5 times in a month, once weekly at 1000 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 1000 mg, followed by administration 5 times in a month, once weekly at 1000 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 140 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 420 mg, followed by administration once-monthly, about every 28 days at 4000 mg, for up to about 2 years. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 1000 mg, followed by administration once-monthly, about every 28 days at 4000 mg, for up to about 2 years. In some embodiments, the treatment continues for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 140 mg, followed by administration 5 times in a month, once weekly at 1000 mg, followed by administration once-monthly, about every 28 days at 4000 mg, for the life of the patient. In some embodiments, a therapeutic formulation comprising 20 mg/ml (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 420 mg, followed by administration 5 times in a month, once weekly at 1000 mg, followed by administration once-monthly, about every 28 days at 4000 mg, for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 1000 mg, followed by administration 5 times in a month, once weekly at 1000 mg, followed by administration once-monthly, about every 28 days at 4000 mg, for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 140 mg, followed by administration once-monthly, about every 28 days at 4000 mg, for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 420 mg, followed by administration once-monthly, about every 28 days at 4000 mg, for the life of the patient. In some embodiments, a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, is administered intravenously over the course of 1-hour, 5 times in a month, once weekly, for a total administered dose of 1000 mg, followed by administration once-monthly, about every 28 days at 4000 mg, for the life of the patient. In some embodiments, administration of a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8, results in an increase in the subject's MMSE score and/or a decrease in the subject's CDR-SB score. In some embodiments, the subject's MMSE score increases by 1, 2, 3, 4, 5, 6, 7, 8, 9, 10, 11, 12, 12, 14, or 15, points, or by a range that is defined by any two of the preceding values. For example, in some embodiments, the subject's MMSE score is increased by about 1-15, 1-10, 1-7, 1-5, 1-3, 3-15, 3-10, 3-7, 3-5, 5-15, 5-10, 5-7, 7-15 7-10, or 10-15, points following one or more administrations of a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8. In some embodiments, the subject's CDR-SB score decreases by 0.1, 0.2, 0.3, 0.4, 0.5, 0.6, 0.7, 0.8, 0.9, 1, 1.5, 2, or 2.5 points, or by a range that is defined by any two of the preceding values. For example, in some embodiments, the subject's CDR-SB score is decreased by about 0.1-2.5, 0.1-2, 0.1-1.5, 0.1-1, 0.1-0.5, 0.5-2.5, 0.5-2, 0.5-1.5, 0.5-1, 1-2.5, 1-2, 1-1.5, 1.5-2.5, 1.5-2, or 2-2.5, following one or more administrations of a therapeutic formulation comprising 20 mg/mL (in some embodiments, the 20 mg/ml can be diluted to 8 mg/ml in a total of 500 mL prior to I. V. administration) TB006, 20 mM L-Histidine, 5 mM Methionine, 100 mM NaCl, 0.02% PS80, and a pH of 5.8. In some embodiments, the dose of antibody administered is between 70 and 4000 mg. For example, in some embodiments, the dose of antibody administered is 70, 100, 140, 250, 420, 500, 600, 700, 750, 800, 900, 1000, 1250, 1500, 1750, 2000, 2250, 2500, 2750, 3000, 3250, 3500, 3750, 4000, 4250, 4500, 4750, or 5000 mg, or a range that is defined by any two of the preceding values. In some embodiments, the dose administered is chosen to achieve an improved MMSE and/or CDR-SB score. In some embodiments, the dose administered is chosen to achieve an increase in the subject's MMSE score of between 1-15 points and a decrease in the subject's CDR-SB score of between 0.1 and 2.5 points.

Some embodiments provided herein relate to a method of treating a patient, the method comprising: identifying a patient, wherein the patient is identified as having a risk of a decreased MMSE score, decreased EQ-5D-5L score, increased CDR-SB score, decreased NPI score, decreased Cognitive Drug Research battery, increased brain atrophy, decreased whole brain volume, increased symptoms related to aging and/or aging related senescence, decreased cognitive ability, decreased strength, decreased locomotor functions, decreased balance, or any combination thereof, and administering a therapeutically effective amount of an anti Gal3 Ab to the patient to reduce said risk.

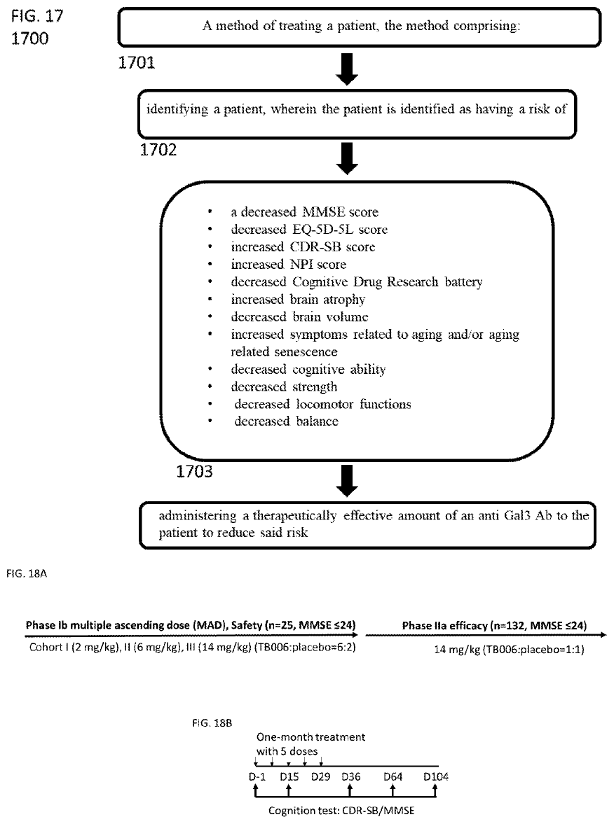

FIG. 17 is a flow chart depicting some embodiments of a method of treating a patient.

In some embodiments, a method of treating a patient 1701 is disclosed. In some embodiments, the method comprises identifying a patient 1702, wherein the patient is identified as having a risk of a decreased MMSE score, decreased EQ-5D-5L score, increased CDR-SB score, decreased NPI score, decreased Cognitive Drug Research battery, increased brain atrophy, decreased whole brain volume, increased symptoms related to aging and/or aging related senescence, decreased cognitive ability, decreased strength, decreased locomotor functions, decreased balance, or any combination thereof 1703, and administering a therapeutically effective amount of an anti Gal3 Ab to the patient to reduce said risk 1704.5

In some embodiments, the patients have one or more diseases. In some embodiments, one or more patients have AD. In some embodiments, the patients are screened based on a baseline assessment of the patient's MMSE score, EQ-5D-5L score, CDR-SB score, NPI score, Cognitive Drug Research battery, level or rate of brain atrophy, whole brain volume, one or more symptoms related to aging and/or aging related senescence, cognitive ability, strength, locomotor, balance, or any combination thereof. In some embodiments, one or more patients are administered an anti-Gal3 antibody. In some embodiments, one or more patients are administered a placebo. In some embodiments, the patients are administered the anti-Gal3 antibody and/or placebo at a consistent dose. In some embodiments, the patients are administered the anti-Gal3 antibody and/or placebo at different doses. In some embodiments, the dosages are sequentially increasing dosages. In some embodiments, the patients are separated into groups. In some embodiments each group gets administered the same dosage of anti-Gal3 antibody or placebo. In some embodiments each group gets administered a different dosage of anti-Gal3 antibody or placebo. In some embodiments, the study is conducted until a predetermined number of doses of anti-Gal3 antibody or placebo have been administered. In some embodiments, the study continues until a desired number or fraction of total patients reaches one or more endpoints. In some embodiments, the one or more endpoints include a mini-mental state examination (MMSE), neuropsychiatric inventory (NPI), Cognitive Drug Research battery, the presence, absence, or level of one or more plasma biomarkers, physical biomarkers, and imaging (MRI/PET) biomarkers. In some embodiments, testing is done at baseline and at predetermined intervals throughout the study. In some embodiments, safety assessments are conducted at each visit. In some embodiments, safety assessments are conducted at defined intervals throughout the study. In some embodiments, one or more patients are prematurely discharged from the study. In some embodiments, the one or more patients are prematurely discharged from the study due to an adverse event, or serious adverse event. In some embodiments, up to about 1, 2, 3, 4, 5, 6, 7, 8, 9, 10, 15, 20, 25, 30, 40, or 50% of patients, or a range that is defined by any two of the preceding values, are prematurely discontinued. In some embodiments, the anti-Gal3 antibody is safe and well tolerated at all dose levels. In some embodiments, the anti-Gal3 antibody is safe and well tolerated at some but not all dose levels. In some embodiments, none of the SAEs are related to the anti-Gal3 antibody treatment. In some embodiments, most AEs were mild, sporadic and self-limiting. In some embodiments, 5, 10, 15, 20, 25, 50, 60, 70, 80, 90, 95, 96, 97, 98, 99, or 100%, or a range that is defined by any two of the preceding values, of anti-Gal3 related AEs are mild, sporadic and self-limiting. In some embodiments, treatment and placebo patients are included in the efficacy analysis. In some embodiments, patients receiving anti-Gal3 antibody treatment show improvement in the subject's MMSE score, EQ-5D-5L score, CDR-SB score, NPI score, Cognitive Drug Research battery, level or rate of brain atrophy, whole brain volume, one or more symptoms related to aging and/or aging related senescence, cognitive ability, strength, locomotor, balance, or any combination thereof. In some embodiments, the study includes one or more secondary efficacy endpoints. In some embodiments, mean efficacy endpoint scores in the placebo group remains consistent throughout the observation period. In some embodiments, anti-Gal3 antibody demonstrates evidence of AD reversal in subjects participating in a short-term, and/or long-term treatment study. In some embodiments, the anti-Gal3 antibody demonstrates evidence of improved cognition in in subjects participating in a short-term and/or long-term treatment study.

In some embodiments, a method of treating microglia mediated inflammation in a subject in need thereof is disclosed. In some embodiments, the method comprises: identifying a subject as having or at risk of having microglia mediated inflammation and administering a therapeutically effective amount of an anti-Gal3 antibody to the subject, wherein administration of the antibody to the subject alleviates one or more symptoms of microglia mediated inflammation.

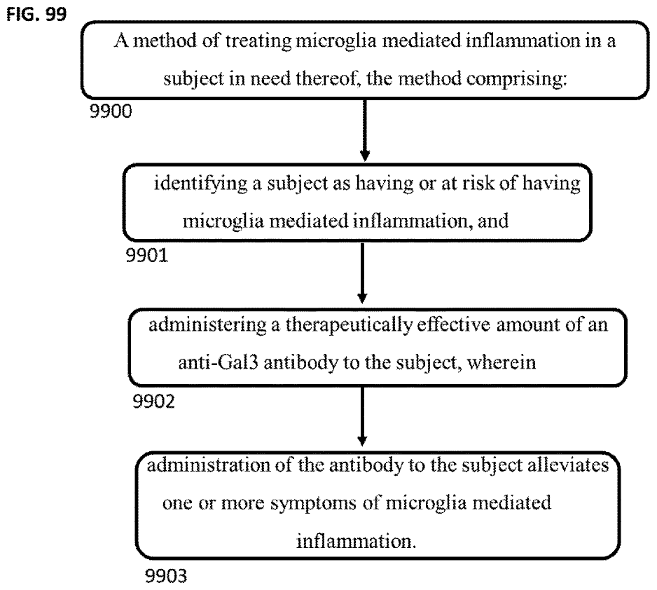

FIG. 99 is a flowchart depicting some embodiments of a method of treating microglia mediated inflammation.

In some embodiments, a method of treating microglia mediated inflammation 9900 in a subject in need thereof is disclosed. In some embodiments, the method comprises: identifying a subject as having or at risk of having microglia mediated inflammation 9901 and administering a therapeutically effective amount of an anti-Gal3 antibody to the subject 9902, wherein administration of the antibody to the subject alleviates one or more symptoms of microglia mediated inflammation 9903. The anti-Gal3 antibody may be administered through any effective means, for example, intravenously, intraperitoneally, or topically. In some embodiments, the microglia mediated inflammation is a cause or symptom of one or more diseases, disorders, or disabilities, including AD. related senescence, cognitive ability, strength, locomotor, balance, or any combination thereof. In some embodiments, one or more patients are administered an anti-Gal3 antibody. In some embodiments, one or more patients are administered a placebo. In some embodiments, the patients are administered the anti-Gal3 antibody and/or placebo at a consistent dose. In some embodiments, the patients are administered the anti-Gal3 antibody and/or placebo at different doses. In some embodiments, the dosages are sequentially increasing dosages. In some embodiments, the patients are separated into groups. In some embodiments each group gets administered the same dosage of anti-Gal3 antibody or placebo. In some embodiments each group gets administered a different dosage of anti-Gal3 antibody or placebo. In some embodiments, the study is conducted until a predetermined number of doses of anti-Gal3 antibody or placebo have been administered. In some embodiments, the study continues until a desired number or fraction of total patients reaches one or more endpoints. In some embodiments, the one or more endpoints include a mini-mental state examination (MMSE), neuropsychiatric inventory (NPI), Cognitive Drug Research battery, the presence, absence, or level of one or more plasma biomarkers, physical biomarkers, and imaging (MRI/PET) biomarkers. In some embodiments, testing is done at baseline and at predetermined intervals throughout the study. In some embodiments, safety assessments are conducted at each visit. In some embodiments, safety assessments are conducted at defined intervals throughout the study. In some embodiments, one or more patients are prematurely discharged from the study. In some embodiments, the one or more patients are prematurely discharged from the study due to an adverse event, or serious adverse event. In some embodiments, up to about 1, 2, 3, 4, 5, 6, 7, 8, 9, 10, 15, 20, 25, 30, 40, or 50% of patients, or a range that is defined by any two of the preceding values, are prematurely discontinued. In some embodiments, the anti-Gal3 antibody is safe and well tolerated at all dose levels. In some embodiments, the anti-Gal3 antibody is safe and well tolerated at some but not all dose levels. In some embodiments, none of the SAEs are related to the anti-Gal3 antibody treatment. In some embodiments, most AEs were mild, sporadic and self-limiting. In some embodiments, 5, 10, 15, 20, 25, 50, 60, 70, 80, 90, 95, 96, 97, 98, 99, or 100%, or a range that is defined by any two of the preceding values, of anti-Gal3 related AEs are mild, sporadic and self-limiting. In some embodiments, treatment and placebo patients are included in the efficacy analysis. In some embodiments, patients receiving anti-Gal3 antibody treatment show improvement in the subject's MMSE score, EQ-5D-5L score, CDR-SB score, NPI score, Cognitive Drug Research battery, level or rate of brain atrophy, whole brain volume, one or more symptoms related to aging and/or aging related senescence, cognitive ability, strength, locomotor, balance, or any combination thereof. In some embodiments, the study includes one or more secondary efficacy endpoints. In some embodiments, mean efficacy endpoint scores in the placebo group remains consistent throughout the observation period. In some embodiments, anti-Gal3 antibody demonstrates evidence of AD reversal in subjects participating in a short-term, and/or long-term treatment study. In some embodiments, the anti-Gal3 antibody demonstrates evidence of improved cognition in in subjects participating in a short-term and/or long-term treatment study. In some embodiments, the patients are screened based on a baseline assessment of the patient's plasma Gal3 levels, plasma Aβ levels, Aβ plaques, Gal3 concentration on blood vessels surrounding Aβ plaques, MMSE score, EQ-5D-5L score, CDR-SB score, NPI score, Cognitive Drug Research battery, level or rate of brain atrophy, whole brain volume, one or more symptoms related to aging and/or aging related senescence, cognitive ability, strength, locomotor, balance, or any combination thereof. In some embodiments, identifying a subject at risk of having microglia mediated inflammation may comprise identifying the subject as at risk of having AD. In some embodiments, the subject's are screened based on a baseline assessment of the subject's plasma Gal3 levels, plasma Aβ levels, Aβ plaques, Gal3 concentration on blood vessels surrounding Aβ plaques, MMSE score, EQ-5D-5L score, CDR-SB score, NPI score, Cognitive Drug Research battery, level or rate of brain atrophy, whole brain volume, one or more symptoms related to aging and/or aging related senescence, cognitive ability, strength, locomotor, balance, or any combination thereof. In some embodiments, administration of the anti-Gal3 antibody to the subject may relieve one or more symptoms associated with microglia mediate inflammation. In some embodiments the anti-Gal3 antibody comprises TB001, TB006, or a fragment thereof. In some embodiments, treatment of the microglia mediated inflammation causes or contributes to an increase in cognition. In some embodiments, the microglia mediated inflammation causes or contributes to AD treatment, halting or slowing of AD progression, treatment of one or more AD symptoms, or any combination thereof. In some embodiments, treatment of the microglia mediated inflammation is related to a decrease in a subject's CDR-SB score. In some embodiments, the treatment of the microglia mediated inflammation causes or contributes to an increase in a subject's MMSE score. In some embodiments, treatment of the microglia mediated inflammation causes or contributes to an increase in a subject's EQ-5D-5L score. In some embodiments, treatment of the microglia mediated inflammation causes or contributes to a decrease in a subject's NPI score. In some embodiments, treatment of the microglia mediated inflammation causes or contributes to a decrease in a subject's CDR-SB score. In some embodiments, treatment of the microglia mediated inflammation causes or contributes to an increase in a subject's cognitive drug research battery score. In some embodiments, treatment of the microglia mediated inflammation causes or contributes to an increase in a subject's strength, locomotor functions, and/or balance. In some embodiments, treatment of the microglia mediated inflammation causes or contributes to a decrease in the frequency and/or severity of symptoms related to aging and/or aging related senescence. In some embodiments, treatment of the microglia mediated inflammation causes or contributes to an increase in a subject's brain volume. In some embodiments, treatment of the microglia mediated inflammation causes or contributes to a decrease in brain atrophy in the subject. In some embodiments, treatment of the microglia mediated inflammation causes or contributes to an increase in the subject's performance on one or more standard cognitive assessments.

In some embodiments, microglia activation or microglia mediated inflammation decrease by about 1, 2, 3, 4, 5, 6, 7, 8, 9, 10, 11, 12, 13, 14, 15, 20, 25, 30, 40, 50, 60, 70, 75, 80, 90, 100, 150, 200, 250, 300, 400, 500, 600, 700, 750, 800, 900, or 1000%, or by a range that is defined by any two of the preceding values, following administration of the anti-Gal3 antibody as compared to microglia activation or microglia mediated inflammation in the subject prior to administration of the antibody. For example, in some embodiments, microglia activation or microglia mediated inflammation decrease by between about 1-1000, 1-750, 1-500, 1-250, 1-150, 1-100, 1-75, 1-50, 1-25, 1-10, 1-5, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000%, following administration of the anti-Gal3 antibody as compared to microglia activation or microglia mediated inflammation in the subject prior to administration of the antibody.

In some embodiments, microhemorrhages in the subject decrease by about 1, 2, 3, 4, 5, 6, 7, 8, 9, 10, 11, 12, 13, 14, 15, 20, 25, 30, 40, 50, 60, 70, 75, 80, 90, 100, 150, 200, 250, 300, 400, 500, 600, 700, 750, 800, 900, or 1000%, or by a range that is defined by any two of the preceding values, following administration of the anti-Gal3 antibody as compared to microhemorrhages in the subject prior to administration of the antibody. For example, in some embodiments, microhemorrhages decrease by between about 1-1000, 1-750, 1-500, 1-250, 1-150, 1-100, 1-75, 1-50, 1-25, 1-10, 1-5, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000%, following administration of the anti-Gal3 antibody as compared to microhemorrhages in the subject prior to administration of the antibody.

In some embodiments, the subjects are at least about 25, 30, 40, 50, 55, 56, 60, 65, 70, 71, 72, 72.4, 73, 74, 75, 80, 85, 88, 90, 95, 100, 105, 110, or 115 years old, or an age that is in a range defined by any two of the preceding values. For example, in some embodiments, the subjects are at least about 25-115, 25-100, 25-95, 25-90, 25-75, 25-70, 25-50, 50-115, 50-111, 50-95, 50-90, 50-75, 50-60, 56-88, 60-115, 60-100, 60-95, 60-90, 60-80, 60-70, 70-115, 70-100, 70-95, 70-90, or 90-115 years old. In some embodiments, the age is between 50-90, such as 56-88.

As shown in the examples, provided herein is a study that confirms the use of anti-Gal3 antibodies in improving one or more of the subject's MMSE score, EQ-5D-5L score, CDR-SB score, NPI score, Cognitive Drug Research battery, level or rate of brain atrophy, whole brain volume, one or more symptoms related to aging and/or aging related senescence, cognitive ability, strength, locomotor, balance, or any combination thereof. The study is a seamless Ph 1b/2a double-blinded, placebo controlled, multicenter study. AD patients with a screening MMSE<24 and without confounding neurologic or psychiatric disease are eligible. In Part 1, 3 groups (140 mg, 420 mg, 1000 mg) of 8 patients in sequential escalating fashion receive either weekly TB006 (6) or placebo (2) infusions for 5 doses. In Part 2, participants are randomized (1:1) to receive either TB006 (1000 mg) or placebo weekly for 5 doses. Part 2 uses the clinical dementia rating-sum of boxes (CDR-SB) score as the primary endpoint. Other potential endpoints are the mini-mental state examination (MMSE), neuropsychiatric inventory (NPI), CDR battery and plasma and imaging (MRI/PET) biomarkers. Cognition testing is done at baseline and on Days 15, 36, 64, and 104. Safety assessments are conducted at each visit. The sample size provides 80% power to detect a mean difference between TB006 and placebo of 0.25 point at Day 104 on the CDR-SB.

In the study described in the examples herein, 157 patients, including 24 in Part 1, are randomized at 15 US sites. Nine subjects prematurely discontinued. TB006 is safe and well tolerated at all dose levels. There are 9 severe adverse events (SAEs), including 1 death. None of the SAEs, including the 1 death, are related to TB006 treatment. Most other AEs are mild, sporadic and self-limiting. Patients in Part 1, Group 3 (1000 mg), as well as all Part 1 placebo patients are included in the efficacy analysis. The primary endpoint is met. Patients receiving TB006 show a dramatic reduction on the CDR-SB score compared with placebo (p<0.01). Secondary efficacy endpoints are equally robust. Mean efficacy endpoint scores in the placebo group remain consistent throughout the observation period. The conclusion is reached that TB006 demonstrates evidence of AD reversal in this short-term treatment study. TB006 is determined to be safe and well tolerated. TB006 is determined to improve subject's score on one or more cognitive assessments.

In some embodiments, the anti-Gal3 antibodies or binding fragments thereof disclosed herein are administered for therapeutic applications, such as in embodiments of the methods disclosed herein. In some embodiments, the anti-Gal3 antibody or binding fragment thereof is administered once per day, twice per day, three times per day or more. The anti-Gal3 antibody or binding fragment thereof is administered daily, every day, every alternate day, five days a week, once a week, every other week, two weeks per month, three weeks per month, once a month, twice a month, three times per month, or more. In some embodiments, the anti-Gal3 antibody or binding fragment thereof is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the anti-Gal3 antibody or binding fragment thereof is given continuously; alternatively, the dose of the anti-Gal3 antibody or binding fragment thereof being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's condition has occurred, a maintenance dose is administered if necessary. Subsequently, the dosage or the frequency of administration, or both, can be reduced, as a function of the symptoms, to a level at which the treated disease, disorder, or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

In some embodiments, the one or more cognitive assessments comprise a MMSE, CDR-SB, EQ-5D-5L, NPI, a Cognitive Drug Research battery, or any combination thereof. In some embodiments, a MMSE, CDR-SB, EQ-5D-5L, NPI, Cognitive Drug Research battery, or any combination thereof, is performed on the subject prior to administration of the antibody.

In some embodiments, a MMSE, CDR-SB, EQ-5D-5L, NPI, Cognitive Drug Research battery, or any combination thereof, is performed on the subject up to 1, 5, 10, 15, 28, 29, 30, 31, 36, 40, 45, 50, 55, 60, 64, 65, 70, 75, 80, 85, 90, 95, 100, 104, 105, 110, 120, 130, 140, 150, 160, 170, or 180 days after administration of the antibody, or at intervals that are defined by any two of the preceding values. For example, in some embodiments, the MMSE, CDR-SB, EQ-5D-5L, NPI, Cognitive Drug Research battery, or any combination thereof, is performed on the subject up to 1-180, 1-120, 1-90, 1-60, 1-31, 1-30, 1-29, 1-28, 28-180, 28-120, 28-90, 28-26, 28-31, 31-180, 31-150, 31-120, 31-90, 31-60, 60-180, 6-150, 6-120, 60-90, 90-180, 90-150, 90-120, or 120-180 days. In some embodiments, the subject's score on the MMSE, CDR-SB, EQ-5D-5L, NPI, Cognitive Drug Research battery, or any combination thereof is improved, i.e., increased or decreased, by at least 5, 10, 15, 20, 25, 30, 40, 50, 60, 70, 75, 80, 90, 95, 96, 97, 98, 99, 100, 200, 250, 300, 400, 500, 750, or 1000%, or by a range that is defined by any two of the preceding values, as compared to the subject's score on a MMSE, CDR-SB, EQ-5D-5L, NPI, Cognitive Drug Research battery, or any combination thereof, conducted up to at least 1, 5, 10, 15, 28, 29, 30, 31, 36, 40, 45, 50, 55, 60, 64, 65, 70, 75, 80, 85, 90, 95, 100, 104, 105, 110, 120, 130, 140, 150, 160, 170, or 180 days prior to administration of the antibody. For example, in some embodiments, the subject's score on the MMSE, CDR-SB, EQ-5D-5L, NPI, Cognitive Drug Research battery, or any combination thereof is improved; i.e. increased or decreased, by at least 5-1000, 5-750, 5-500, 5-250, 5-100, 5-95, 5-90, 5-75, 5-50, 5-25, 5-10, 10-100, 10-95, 10-90, 10-75, 10-50, 10-25, 25-100, 25-95, 25-90, 25-75, 25-50, 50-100, 50-95, 50-90, 50-75, 75-100, 75-95, or 75-90%, as compared to the subject's score on a MMSE, CDR-SB, EQ-5D-5L, NPI, Cognitive Drug Research battery, or any combination thereof, conducted up to at least up to 1, 5, 10, 15, 28, 29, 30, 31, 36, 40, 45, 50, 55, 60, 64, 65, 70, 75, 80, 85, 90, 95, 100, 104, 105, 110, 120, 130, 140, 150, 160, 170, or 180 days prior to administration of the antibody. In some embodiments, the subject's score on the MMSE, CDR-SB, EQ-5D-5L, NPI, Cognitive Drug Research battery, or any combination thereof is improved; i.e. increased or decreased, by at least 1-fold, 2-fold, 3-fold, 4-fold, 5-fold, 6-fold, 7-fold, 8-fold, 9-fold, or 10-fold, or by a range that is defined by any two of the preceding values, as compared to the subject's score on a MMSE, CDR-SB, EQ-5D-5L, NPI, Cognitive Drug Research battery, or any combination thereof, conducted up to at least 1, 5, 10, 15, 28, 29, 30, 31, 36, 40, 45, 50, 55, 60, 64, 65, 70, 75, 80, 85, 90, 95, 100, 104, 105, 110, 120, 130, 140, 150, 160, 170, or 180 days prior to administration of the antibody. For example, in some embodiments, the subject's score on the MMSE, CDR-SB, EQ-5D-5L, NPI, Cognitive Drug Research battery, or any combination thereof is improved; i.e. increased or decreased, by at least 1-10, 1-7, 1-5, 1-3, 3-10, 3-7, 3-5, 5-10, or 5-7-fold, as compared to the subject's score on one or more MMSE, CDR-SB, EQ-5D-5L, NPI, Cognitive Drug Research battery, or any combination thereof, conducted up to 1, 5, 10, 15, 28, 29, 30, 31, 36, 40, 45, 50, 55, 60, 64, 65, 70, 75, 80, 85, 90, 95, 100, 104, 105, 110, 120, 130, 140, 150, 160, 170, or 180 days prior to administration of the antibody.

In some embodiments, a MMSE, CDR-SB, EQ-5D-5L, NPI, Cognitive Drug Research battery, or any combination thereof, on the subject at least once every 1, 2, 3, 4, 5, 6, 7, 8, 9, 10, 11, or 12 months after administration of the antibody, or at intervals that are defined by any two of the preceding values. For example, in some embodiments, a MMSE, CDR-SB, EQ-5D-5L, NPI, Cognitive Drug Research battery, or any combination thereof, on the subject at least once every 1-12, 1-9, 1-6, 1-3, 3-12, 3-9, 3-6, 6-12, 6-9, or 9-12 months after administration of the antibody. In some embodiments, the subject's score on the MMSE, CDR-SB, EQ-5D-5L, NPI, Cognitive Drug Research battery, or any combination thereof is improved, i.e., increased or decreased, by at least 5, 10, 15, 20, 25, 30, 40, 50, 60, 70, 75, 80, 90, 95, 96, 97, 98, 99, 100, 200, 250, 300, 400, 500, 750, or 1000%, or by a range that is defined by any two of the preceding values, as compared to the subject's score on a MMSE, CDR-SB, EQ-5D-5L, NPI, Cognitive Drug Research battery, or any combination thereof, conducted 1, 2, 3, 4, 5, 6, 7, 8, 9, 10, 11, or 12 months prior to administration of the antibody. For example, in some embodiments, the subject's score on the MMSE, CDR-SB, EQ-5D-5L, NPI, Cognitive Drug Research battery, or any combination thereof is improved; i.e. increased or decreased, by at least 5-1000%, 5-750, 5-500, 5-250, 5-100, 5-95, 5-90, 5-75, 5-50, 5-25, 5-10, 10-100, 10-95, 10-90, 10-75, 10-50, 10-25, 25-100, 25-95, 25-90, 25-75, 25-50, 50-100, 50-95, 50-90, 50-75, 75-100, 75-95, or 75-90%, as compared to the subject's score on a MMSE, CDR-SB, EQ-5D-5L, NPI, Cognitive Drug Research battery, or any combination thereof, conducted 1, 2, 3, 4, 5, 6, 7, 8, 9, 10, 11, or 12 months prior to administration of the antibody. In some embodiments, the subject's score on the MMSE, CDR-SB, EQ-5D-5L, NPI, Cognitive Drug Research battery, or any combination thereof is improved; i.e. increased or decreased, by at least 1-fold, 2-fold, 3-fold, 4-fold, 5-fold, 6-fold, 7-fold, 8-fold, 9-fold, or 10-fold, or by a range that is defined by any two of the preceding values, as compared to the subject's score on a MMSE, CDR-SB, EQ-5D-5L, NPI, Cognitive Drug Research battery, or any combination thereof, conducted 1, 2, 3, 4, 5, 6, 7, 8, 9, 10, 11, or 12 months prior to administration of the antibody. For example, in some embodiments, the subject's score on the MMSE, CDR-SB, EQ-5D-5L, NPI, Cognitive Drug Research battery, or any combination thereof is improved; i.e. increased or decreased, by at least 1-10, 1-7, 1-5, 1-3, 3-10, 3-7, 3-5, 5-10, or 5-7-fold, as compared to the subject's score on one or more MMSE, CDR-SB, EQ-5D-5L, NPI, Cognitive Drug Research battery, or any combination thereof, conducted 1, 2, 3, 4, 5, 6, 7, 8, 9, 10, 11, or 12 months prior to administration of the antibody.

In some embodiments, the subject has dementia. In some embodiments, the subject has Alzheimer's disease, vascular dementia, Dementia With Lewy Bodies (DLB), Parkinson's Disease Dementia. Mixed Dementia, Frontotemporal Dementia (FTD), Huntington's Disease, Creutzfeldt-Jakob Disease, normal pressure hydrocephalus, and Wernicke-Korsakoff Syndrome.

In some embodiments, the subject has Alzheimer's disease. In some embodiments, the subject is identified as having Alzheimer's disease. In some embodiments, identifying a subject as having AD comprises an assessment based on the National Institute of Neurological and Communicative Disorders and Stroke-Alzheimer's Disease and Related Disorders Association (NINCDS-ADRDA)-Criteria for Diagnosis of Probable Alzheimer's Disease). In some embodiments, the criteria for diagnosis of probable AD includes identifying the subject as having dementia established by clinical examination and documented by a standard test of cognitive function (e.g., MMSE, Blessed Dementia Scale, etc.) and confirmed by neuropsychological tests. In some embodiments, the subject is identified as having significant deficiencies in 2 or more areas of cognition, for example, word comprehension and task-completion ability. In some embodiments, the subject is identified as having progressive deterioration of memory and other cognitive functions with no loss of consciousness. In some embodiments, the identification of a subject as having AD, includes an assessment of the subject's age. In some embodiments, the subject has no other diseases or disorders that could account for the loss of memory and cognition.

Diagnosis of a subject as probably having AD is supported by progressive deterioration of specific cognitive functions: language (aphasia), motor skills (apraxia), and perception (agnosia); impaired activities of daily living and altered patterns of behavior; a family history of similar problems, particularly if confirmed by neurological testing; the following laboratory results: CSF (lumbar puncture test); normal electroencephalogram test of brain activity; evidence of cerebral atrophy in a series of CT scans.

Other features consistent with AD include plateaus in the course of illness progression; CT findings normal for the person's age; associated symptoms including depression, insomnia, incontinence, delusions, hallucinations, weight loss, sex problems, and significant verbal, emotional, and physical outbursts; other neurological abnormalities, especially in advanced disease, including increased muscle tone and a shuffling gait.

Features that decrease the likelihood of AD include sudden onset; such early symptoms as seizures, gait problems, and loss of vision and coordination.

In some embodiments, the subject has, or has had, inflammation, an autoimmune disorder, diabetes, cardiovascular disease, organ dysfunction, organ damage, kidney dysfunction, kidney damage, liver dysfunction, liver damage, heart dysfunction, heart damage, heart failure, a bone disorder, anemia, a hematopoietic disorder, a metabolic disease, cancer, an immune deficiency, neurodegenerative disease, Parkinson's disease, Alzheimer's disease, fibrosis, frailty, respiratory system disease or disorder, or any combination thereof.

In some embodiments, one or more of the subject's biomarkers is monitored following antibody administration.

Table 1 lists some embodiments of biomarkers that may be used in the systems/kits/methods provided herein.

TABLE 1

| | | | rc- | c- |
|---|---|---|---|---|
| Potential biomarkers | Material | Age linked processes | score | score |
| Lymphocytes/WBC [CDC] | blood/EDTA | Inflammation | 202 | 2240 |
| [PA] | | autoimmune disorders | | |
| Insulin | blood/serum | Diabetic state | 148 | 1143 |
| Glucose/glucose fastened | blood/glucose | Diabetic state | 111 | 1175 |
| [PA] | monovette | | | |
| C-reactive protein | blood/plasma | Inflammation, cancer, | 71 | 1146 |
| (CRP/hsCRP) [IA] [PA] | | cardiovascular disease | | |
| Cholesterol | blood/plasma | Cardiovascular disease | 67 | 896 |
| Albumin [PA] | blood/plasma | Kidney and liver | 65 | 1062 |
| | | dysfunction | | |
| IL6 [IA] | blood/plasma | Inflammation | 58 | 979 |
| Tumor necrosis factor alpha | blood/serum | Inflammation, cancer | 51 | 751 |
| (TNFα) [IA] | | | | |
| Hemoglobin [CDC] | blood/EDTA | Anemia, other | 39 | 471 |
| | | hematopoietic | | |
| | | disorders | | |
| Insulin-like growth factor 1 | blood/serum | Metabolic disease | 29 | 263 |
| (IGF-1) | | | | |
| LDL-cholesterol | blood/plasma | Cardiovascular disease | 24 | 280 |
| Triglycerides | blood/plasma | Cardiovascular disease | 23 | 498 |
| HDL-cholesterol | blood/plasma | Cardiovascular disease | 23 | 349 |
| Creatinine [PA] | blood/plasma | Kidney dysfunction | 19 | 479 |
| Monocytes | blood/EDTA | Inflammation | 16 | 378 |
| Glycated hemoglobin | blood/EDTA | Diabetic state | 13 | 220 |
| (Hba1c) | | | | |
| Cystatin C | blood/plasma | Kidney dysfunction | 12 | 142 |
| N-terminal prohormone of | blood/EDTA | Heart failure | 10 | 119 |
| brain natriuretic peptide (NT- | | | | |
| proBNP) | | | | |
| Alkaline phosphatase [PA] | blood/plasma | Liver damage, bone | 9 | 252 |
| | | disorder | | |
| Hematocrit/RBC [CDC] | blood/EDTA | Anemia | 8 | 159 |
| D-dimer | blood/citrate | Hypercoagulable state | 8 | 91 |
| | monovette | | | |
| IL8 [IA] | blood/plasma | Inflammation | 7 | 164 |
| Plasminogen activator | blood/EDTA | Prothrombotic state in | 6 | 72 |
| inhibitor-1 (PAI1) | | cancer and other acute | | |
| | | phases | | |
| Bilirubin | blood/plasma | Liver dysfunction | 5 | 46 |
| Urea | blood/plasma | Renal dysfunction | 3 | 137 |
| IL15 | blood/plasma | Inflammation | 3 | 55 |
| Mean corpuscular | blood/EDTA | Anemia, other | 2 | 42 |
| volume/MCV [CDC] [PA] | | hematopoietic | | |
| | | disorders | | |
| Mean corpuscular | blood/EDTA | Anemia, other | 2 | 32 |
| hemoglobin | | hematopoietic | | |
| concentration/MCHC [CDC] | | disorders | | |
| CD4/CD8 ratio | blood/EDTA | Immune deficiency, | 1 | 103 |
| | | autoimmunity | | |
| C-peptide (preferable to | blood/serum | Diabetic state | 1 | 32 |
| insulin) | | | | |
| IL1-β [IA] | blood/plasma | inflammation | 1 | 5 |

Table 2 lists some embodiments of biomarkers that may be used in the systems/kits/methods provided herein.

TABLE 2

| Potential | | | | rc- | c- |
|---|---|---|---|---|---|
| biomarkers | Material | Methods | Age linked processes | score | score |
| Telomere | | | Morbidity, mortality, | 191 | 932 |
| length (TL): | | | cell stress | | |
| Average TL | DNA | Q-PCR, TRF, TCA | | | ** |
| TL structure | DNA | Q-FISH, Flow- | | | ** |
| | | FISH | | | |
| Shortest TL | DNA | STELA, TeSLA | | | ** |
| DNA damage | DNA | Various methods | Morbidity, mortality | 174 | 713 |
| Reactive | Tissue mitochondria | Various methods | Morbidity, cell stress, | 168 | 712 |
| oxygen species | | | DNA/protein damage | | |
| (ROS) | | | | | |
| Mitochondrial | living cells, | Various methods | Morbidity, mortality, | 86 | 289 |
| dysfunction | mitochondrial DNA | | neurodegenerative | | |
| | | | diseases | | |
| Evs | blood/plasma, | Immuno- | Cellular senescence, | 65 | 194 |
| (extracellular | liquor, cell culture | histochemistry | cancer | | |
| vesicles) | supernatant | Western Blot, | | | |
| | | FACS | | | |
| Autophagy | cells, cell extract | Electron | Morbidity, cancer, | 46 | 207 |
| | | microscopy | Parkinson's and | | |
| | | immunoblotting | Alzheimer's disease | | |
| | | flow cytometry | | | |
| Transforming | blood/serum | ELISA | Inflammation, | 45 | 315 |
| growth factor | | | fibrosis, cellular | | |
| beta (TGF-β) | | | senescence, cancer | | |
| Telomerase | cell extract, | PCR-ELIDA, | Morbidity, mortality, | 41 | 169 |
| activity | DNA | TRAP | tumor progression | | |
| Gut | fecal specimen | Next generation | Morbidity, mortality | 29 | 101 |
| microbiome | | sequencing | | | |
| α-Klotho | blood/plasma tissue | Immuno- | Morbidity, mortality, | 20 | 107 |
| | | histochemistry | renal function | | |
| | | ELISA | | | |
| Adiponectin | blood/plasma | ELISA | Morbidity, mortality, | 14 | 217 |
| | blood/EDTA | | frailty, metabolic | | |
| | | | syndrome, liver | | |
| | | | cirrhosis, diabetes | | |
| | | | type 2 | | |
| Sirtuin 1 | blood/serum | ELISA immuno- | Morbidity, mortality, | 12 | 112 |
| (SIRT1) | | histochemistry | inflammation, cancer | | |
| | | PCR | | | |
| Growth | blood/plasma | Proteomics | Morbidity, organ | 12 | 63 |
| differentiation | | immunoassays | damage (liver, heart, | | |
| factor 15 | | | kidney) | | |
| (GDF15) | | | | | |
| Sirtuin6 | blood/serum | ELISA immuno- | Morbidity, mortality, | 4 | 50 |
| (SIRT6) | | histochemistry | diabetic risk, arthritis | | |
| | | PCR | | | |
| Growth | blood/plasma | Proteomics | Morbidity | 3 | 22 |
| differentiation | | immunoassays | | | |
| factor 11 | | | | | |
| (GDF11) | | | | | |
| CXCL1 | blood/plasma | Immunoassays, | Immune response, | 0 | 15 |
| | | ELISA | inflammation, cancer, | | |
| | | | Alzheimer's disease | | |
| Skin | skin swab | Next generation | Morbidity, mortality | 0 | 4 |
| microbiome | | sequencing | | | |

Table 3 lists some embodiments of biomarkers that that may be used in the systems/kits/methods provided herein.

TABLE 3

| Potential | | | | rc- | c- |
|---|---|---|---|---|---|
| biomarkers | Material | Methods | Prediction | score | score* |
| DNA methylation and | | | | n.a. | 2158 |
| aging clocks: | | | | | |
| Horvath's clock | DNA (broad | DNA methylation analysis | Chronological | n.a. | 214 |
| | spectrum of tissues) | | age | | |
| Hannum's clock | DNA (blood) | | Chronological | n.a. | 190 |
| | | | age | | |
| DNAm GrimAge | DNA (blood) | | Biological age | n.a. | 31 |
| DNAm PhenoAge | DNA (blood) | | Biological age | n.a. | 26 |
| Weidner clock | DNA (blood) | | Chronological | n.a. | 8 |
| | | | age | | |
| EpiTOC | DNA (blood) | | Biological age | n.a. | 2 |
| miRNA (microRNA) | RNA (blood/plasma | Next generation sequencing | Morbidity, | 198 | 635 |
| | PBMCs) | microarrays | mortality | | |
| Non-coding RNA | RNA | RNA sequencing | Chronological | 167 | 602 |
| expression profiles | | | age | | |
| exRNA (extracellular | blood/plasma | Next generation sequencing | Morbidity, | 25 | 119 |
| RNA) | | | mortality | | |
| Histone modifications: | | | | 36 | 73 |
| H4K20 methylation | | DNA methylation analysis mass | Cell stress | n.a. | n.a. |
| | | spectrometry, HPLC, ChIP | | | |
| | | Immunohisto-chemistry | | | |
| H4K16 acetylation | | | | n.a. | n.a. |
| H3K4 methylation | protein extract | | | n.a. | n.a. |
| H3K9 methylation | from tissue DNA | | | n.a. | n.a. |
| H3K27 methylation | | | | n.a. | n.a. |
| Chromatin remodeling | DNA | Chromatin remodeling assays | Chronological | 13 | 26 |
| | | | age | | |

Table 4 lists some embodiments of biomarkers that may be used in the systems/kits/methods provided herein.

TABLE 4

| | | | | | |
|---|---|---|---|---|---|
| Potential | | | | rc- | c- |
| biomarkers | Method | Age linked processes^{#} | Domain | score* | score |

Physical capability

| | | | | | |
|---|---|---|---|---|---|
| Grip strength | Physical exam | Mortality, morbidity | Strength | 11 | 229 |
| Walking speed | Physical exam | Mortality, morbidity | Locomotor | 3 | 106 |
| | | | function | | |
| Standing balance | Physical exam | Mortality, morbidity | Balance | 1 | 26 |
| Timed up and go | Physical exam | Mortality, morbidity | Locomotor | 0 | 11 |
| test | | | function | | |

Organ function

| | | | | | |
|---|---|---|---|---|---|
| Atherosclerotic | IMT, ultrasound | Mortality, CAD | Cardiovascular | 158 | 680 |
| lesions | | | system | | |
| Muscle mass | MRI | Mortality, | Body composition | 81 | 495 |
| | | cardiovascular risk | | | |
| Systolic blood | Auscultatory | Mortality, | Cardiovascular | 65 | 844 |
| pressure | method | cardiovascular risk | system | | |
| Cognitive function | Various | Mortality, morbidity | Brain function | 56 | 581 |
| Body mass index | Calculated | Mortality CAD | Body composition | 24 | 1280 |
| Bone density | Bone density test | Mortality, morbidity | Body composition | 17 | 84 |
| Lung function | Spirometry | Mortality, morbidity | Respiratory system | 16 | 84 |
| Waist | Tape measure | Mortality, | Body composition | 3 | 202 |
| circumference | | cardiovascular risk | | | |

General well being

| | | | | | |
|---|---|---|---|---|---|
| Health assessments | Questionnaire | Mortality, morbidity | General | n.a. | n.a. |

Table 5 lists some embodiments of biomarkers that may be used in the systems/kits/methods provided herein.

TABLE 5

| | | | rc- | c- |
|---|---|---|---|---|
| Potential biomarker | | Material and Methods | score* | score |
| SASP | | | 442 | 2646 |
| | Cytokines (e.g., IL-6, | ELISA from Serum or EDTA plasma | n.a. | n.a. |
| | IL-7, IL-15) | samples proteomics | | |
| | Chemokines (e.g., IL- | | n.a. | n.a. |
| | 8, CCL3, CCL4) | | | |
| | Growth factors (e.g., | | n.a. | n.a. |
| | GDF-15, activin A) | | | |
| Cell cycle arrest | p53 | qPCR from blood samples/staining of | 66 | 561 |
| | p16 | cultured cells/flow cytometry | 27 | 422 |
| | p21 | NGS/microarray | 21 | 435 |
| SA-βGal | | Microscopy/flow cytometry | 9 | 359 |
| SAHF | Histone fragments | DAPI/heterochromatin staining | 3 | 19 |
| | (H3K9Me2, HP1γ) | | | |
| Lamin B1 | | Immunohistochemistry Western Blot | 0 | 12 |
| Cell morphology | Cell shape | Microscopy of cultured cells | n.a. | n.a. |
| (e.g., progerin) | | | | |

In some embodiments, the one or more biomarkers comprises a biomarker for inflammation, autoimmune disorders, diabetes, cardiovascular disease, organ dysfunction, organ damage, kidney dysfunction, kidney damage, liver dysfunction, liver damage, heart dysfunction, heart damage, heart failure, bone disorder, anemia, hematopoietic disorders, metabolic disease, hypercoagulable state, cancer, cancer progression, tumor formation, tumor progression, prothrombotic state in cancer and other acute phases, immune deficiency, morbidity, mortality, cell stress, DNA/protein damage, neurodegenerative disease, cellular senescence, cancer, Parkinson's disease, Alzheimer's disease, fibrosis, frailty, chronological age, biological age, physical capability, strength, locomotor function, balance, brain function, body composition, cardiovascular system, or respiratory system, and/or any combination of biomarkers thereof. In some embodiments, the one or more biomarkers comprise plasma Aβ40, tau (phosphorylated), NFL, NFH, and/or GAL-3, or any combination thereof. In some embodiments, the one or more morbidity, biomarkers comprise telomere length (TL), average TL, TL structure, shortest TL, DNA damage, Reactive Oxygen Species (ROS), mitochondrial dysfunction, autophagy, telomerase activity, gut microbiome, α-Klotho, adiponectin, sirtuin 1 (SIRT1), growth differentiation factor 15 (GDF15), sirtuin 6 (SIRT6), growth differentiation factor 11 (GDF11), skin microbiome, microRNA (miRNA), extracellular RNA (exRNA), grip strength, walking speed, standing balance, timed up and go test, atherosclerotic lesions, muscle mass, systolic blood pressure, cognitive function, body mass index, bone density, lung function, waist circumference, health assessments, or any combination thereof.

In some embodiments, the one or more biomarkers in a subject treated with TB006 is decreased by at least 1, 2, 3, 4, 5, 10, 15, 20, 25, 30, 40, 50, 60, 70, 75, 80, 90, 95, 96, 97, 98, 99, 100, 150, 200, 250, 300, 400, 500, 600, 700, 750, 800, 900 950, or 1000%, or by a range that is defined by any two of the preceding values. For example, in some embodiments, the one or more biomarkers in a subject treated with TB006 is decreased by at least 1-1000, 1-500, 1-250, 1-100, 1-75, 1-50, 1-25, 1-10, 1-5, 5-1000, 5-750, 5-500, 5-250, 5-100, 5-95, 5-90, 5-75, 5-50, 5-25, 5-10, 10-100, 10-95, 10-90, 10-75, 10-50, 10-25, 25-100, 25-95, 25-90, 25-75, 25-50, 50-100, 50-95, 50-90, 50-75, 75-100, 75-95, or 75-90%. In some embodiments, the one or more biomarkers in a subject treated with TB006 is decreased by at least 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, or 10-fold, or by a range that is defined by any two of the preceding values. For example, in some embodiments, the one or more biomarkers in a subject treated with TB006 is decreased by at least 0.1-10, 0.1-7, 0.1-5, 0.1-3, 0.1-1, 0.1-0.7, 0.1-0.5, 0.1-0.3, 0.3-10, 0.3-7, 0.3-5, 0.3-3, 0.3-1, 0.3-0.7, 0.3-0.5, 0.5-10, 0.5-7, 0.5-5, 0.5-3, 0.5-1, 0.5-0.7, 1-10, 1-7, 1-5, 1-3, 3-10, 3-7, 3-5, 5-10, or 5-7-fold.

In some embodiments, the one or more biomarkers in a subject treated with TB006 is increased by at least 1, 2, 3, 4, 5, 10, 15, 20, 25, 30, 40, 50, 60, 70, 75, 80, 90, 95, 96, 97, 98, 99, 100, 150, 200, 250, 300, 400, 500, 600, 700, 750, 800, 900 950, or 1000%, or by a range that is defined by any two of the preceding values. For example, in some embodiments, the one or more biomarkers in a subject treated with TB006 is increased by at least 1-1000, 1-500, 1-250, 1-100, 1-75, 1-50, 1-25, 1-10, 1-5, 5-1000, 5-750, 5-500, 5-250, 5-100, 5-95, 5-90, 5-75, 5-50, 5-25, 5-10, 10-100, 10-95, 10-90, 10-75, 10-50, 10-25, 25-100, 25-95, 25-90, 25-75, 25-50, 50-100, 50-95, 50-90, 50-75, 75-100, 75-95, or 75-90%. In some embodiments, the one or more biomarkers in a subject treated with TB006 is increased by at least 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, or 10-fold, or by a range that is defined by any two of the preceding values. For example, in some embodiments, the one or more biomarkers in a subject treated with the anti-body is increased by at least 0.1-10, 0.1-7, 0.1-5, 0.1-3, 0.1-1, 0.1-0.7, 0.1-0.5, 0.1-0.3, 0.3-10, 0.3-7, 0.3-5, 0.3-3, 0.3-1, 0.3-0.7, 0.3-0.5, 0.5-10, 0.5-7, 0.5-5, 0.5-3, 0.5-1, 0.5-0.7, 1-10, 1-7, 1-5, 1-3, 3-10, 3-7, 3-5, 5-10, or 5-7-fold.

In some embodiments, the level of one or more biomarkers increases by at least 1, 2, 3, 4, 5, 10, 15, 20, 25, 30, 40, or 50%, or by a range that is defined by any two of the preceding values, within about 1, 5, 7, 8, 10, 14, 15, 21, 28, 29, 30, 31, 36, 59, 60, 61, 64, 89, 90, 91, 100, 104, or 105 days of initial administration of TB006, or a time frame that is defined by any two of the preceding values, and then decreases by at least 1, 2, 3, 4, 5, 10, 15, 20, 25, 30, 40, or 50%, following prolonged treatment with TB006.

In some embodiments, the level of Gal3 biomarker in increases by about 1, 5, 10, 20, 30, 40, 50, 60, 70, 80, 90, 100, 110, 120, 130, 140, 150, 160, 170, or 180 ng/mL, or by a range that is defined by any two of the preceding values. For example, in some embodiments, the level of plasma Gal3 increases by about 1-180, 1-150, 1-120, 1-90, 1-60, 1-30, 1-10, 10-180, 10-150, 10-120, 10-90, 10-60, 10-30, 30-180, 30-150, 30-120, 30-90, 30-60, 60-180, 60-150, 60-120, 60-90, 90-189, 90-150, 90-120, 120-180, 120-150, or 150-108 ng/mL. In some embodiments, plasma Gal3 levels rise following initial administration of anti-Gal3 antibodies as Gal3 is cleared from the brain of AD subjects. In some embodiments, as Gal3 is cleared from the brain, Gal3 plasma levels will initially increase, but will then decrease as there becomes less and less Gal3 to clear. For example, in some embodiments, the level of plasma Gal3 will increase by about 1, 5, 10, 20, 30, 40, 50, 60, 70, 80, 90, 100, 110, 120, 130, 140, 150, 160, 170, or 180 ng/ml, or by a range that is defined by any two of the preceding values, and then decrease by 1, 5, 10, 20, 30, 40, 50, 60, 70, 80, 90, 100, 110, 120, 130, 140, 150, 160, 170, or 180 ng/mL. For example, in some embodiments, the level of plasma Gal3 increases by about 1-180, 1-150, 1-120, 1-90, 1-60, 1-30, 1-10, 10-180, 10-150, 10-120, 10-90, 10-60, 10-30, 30-180, 30-150, 30-120, 30-90, 30-60, 60-180, 60-150, 60-120, 60-90, 90-189, 90-150, 90-120, 120-180, 120-150, or 150-108 ng/ml; and then decrease by about 1-180, 1-150, 1-120, 1-90, 1-60, 1-30, 1-10, 10-180, 10-150, 10-120, 10-90, 10-60, 10-30, 30-180, 30-150, 30-120, 30-90, 30-60, 60-180, 60-150, 60-120, 60-90, 90-189, 90-150, 90-120, 120-180, 120-150, or 150-108 ng/ml.

In some embodiments, the one or more biomarkers comprise Aβ42 levels. In some embodiments, the level of Aβ42 in a subject treated with TB006 is decreased by at least 1, 2, 3, 4, 5, 10, 15, 20, 25, 30, 40, 50, 60, 70, 75, 80, 90, 95, 96, 97, 98, 99, 100, 150, 200, 250, 300, 400, 500, 600, 700, 750, 800, 900 950, or 1000%, or by a range that is defined by any two of the preceding values. For example, in some embodiments, the level of Aβ42 in a subject treated with TB006 is decreased by at least 1-1000, 1-500, 1-250, 1-100, 1-75, 1-50, 1-25, 1-10, 1-5, 5-1000, 5-750, 5-500, 5-250, 5-100, 5-95, 5-90, 5-75, 5-50, 5-25, 5-10, 10-100, 10-95, 10-90, 10-75, 10-50, 10-25, 25-100, 25-95, 25-90, 25-75, 25-50, 50-100, 50-95, 50-90, 50-75, 75-100, 75-95, or 75-90%. In some embodiments, the level of Aβ42 in a subject treated with TB006 is decreased by at least 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, or 10-fold, or by a range that is defined by any two of the preceding values. For example, in some embodiments, the level of Aβ42 in a subject treated with TB006 is decreased by at least 0.1-10, 0.1-7, 0.1-5, 0.1-3, 0.1-1, 0.1-0.7, 0.1-0.5, 0.1-0.3, 0.3-10, 0.3-7, 0.3-5, 0.3-3, 0.3-1, 0.3-0.7, 0.3-0.5, 0.5-10, 0.5-7, 0.5-5, 0.5-3, 0.5-1, 0.5-0.7, 1-10, 1-7, 1-5, 1-3, 3-10, 3-7, 3-5, 5-10, or 5-7-fold. In some embodiments, the level of Aβ42 in a subject treated with TB006 is decreased by at least 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.75, 1.8, 1.9, 2.0, 2.25, or 2.5 pg/mL, or by a range that is defined by any two of the preceding values, as compared to the level of Aβ42 in a subject prior to administration of TB006. For example, in some embodiments, the level of Aβ42 in a subject treated with TB006 is decreased by at least 0.05-2.5, 0.05, −2, 0.05-1.5, 0.05-1, 0.05-0.75, 0.05-0.5, 0.05, 0.25, 0.05-0.1, 0.1-2.5, 0.1-2, 0.1-1.5, 0.1-1, 0.1-0.75, 0.1-0.5, 0.1-0.25, 0.25-2.5, 0.25-2, 0.25-1.5, 0.25-1, 0.25-0.75, 0.25-0.5, 0.5-2.5, 0.5-2, 0.5-1.5, 0.5-1, 0.5-0.75, 0.75-2.5, 0.75-2, 0.75-1.5, 0.75-1, 1-2.5, 1-2, 1-1.5, 1.5-2.5, 1.5-2, or 2-2.5 μg/mL, as compared to the level of Aβ42 in a subject prior to administration of TB006. In some embodiments, a subject treated with TB006 until the level of Aβ42 in in the subject is decreased by at least 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.75, 1.8, 1.9, 2.0, 2.25, or 2.5 μg/mL, or by a range that is defined by any two of the preceding values, as compared to the level of Aβ42 in a subject prior to administration of TB006. For example, in some embodiments, a subject treated with TB006 until the level of Aβ42 in in the subject is decreased by at least 0.05-2.5, 0.05, −2, 0.05-1.5, 0.05-1, 0.05-0.75, 0.05-0.5, 0.05, 0.25, 0.05-0.1, 0.1-2.5, 0.1-2, 0.1-1.5, 0.1-1, 0.1-0.75, 0.1-0.5, 0.1-0.25, 0.25-2.5, 0.25-2, 0.25-1.5, 0.25-1, 0.25-0.75, 0.25-0.5, 0.5-2.5, 0.5-2, 0.5-1.5, 0.5-1, 0.5-0.75, 0.75-2.5, 0.75-2, 0.75-1.5, 0.75-1, 1-2.5, 1-2, 1-1.5, 1.5-2.5, 1.5-2, or 2-2.5 μg/mL, as compared to the level of Aβ42 in a subject prior to administration of TB006. In some embodiments, the level of Aβ42 in a subject treated with TB006 is decreased by at least 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.75, 1.8, 1.9, 2.0, 2.25, or 2.5 μg/mL, or by a range that is defined by any two of the preceding values, within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of TB006. For example, in some embodiments, the level of Aβ42 in a subject treated with TB006 is decreased by at least 0.05-2.5, 0.05, −2, 0.05-1.5, 0.05-1, 0.05-0.75, 0.05-0.5, 0.05, 0.25, 0.05-0.1, 0.1-2.5, 0.1-2, 0.1-1.5, 0.1-1, 0.1-0.75, 0.1-0.5, 0.1-0.25, 0.25-2.5, 0.25-2, 0.25-1.5, 0.25-1, 0.25-0.75, 0.25-0.5, 0.5-2.5, 0.5-2, 0.5-1.5, 0.5-1, 0.5-0.75, 0.75-2.5, 0.75-2, 0.75-1.5, 0.75-1, 1-2.5, 1-2, 1-1.5, 1.5-2.5, 1.5-2, or 2-2.5 μg/mL, within about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of TB006.

In some embodiments, the level of Aβ42 in a subject treated with TB006 is decreased by at least 0.05-1 μg/mL. In some embodiments, the level of Aβ42 in a subject treated with TB006 is decreased by at least 0.05-1 μg/mL within at least 104 days following administration of the TB006.

In some embodiments, the subject is given one or more physical exams. In some embodiments, the physical exam comprises assessments of the cardiovascular, respiratory, gastrointestinal, and neurological systems. In some embodiments, height and weight will also be measured and recorded. In some embodiments, the neurological examination will assess mental status, motor and sensory skills, hearing and speech, vision, coordination, and balance.

In some embodiments the physical exam comprises an electrocardiograms. In some embodiments, ECGs (12-lead) will be obtained using an ECG machine that automatically calculates the heart rate and measures PR, QRS, QT, and QTc intervals. Single measurements are acceptable at all-time points. In some embodiments, on non-dosing days, effort should be made to perform 12-lead ECGs time-matched with the pre-dose time point on dosing days.

In some embodiments, the one or more physical exams comprises measuring one or more of the subject's vital signs. In some embodiments, the vital signs assessed comprise temperature, oral temperature, heart rate, respiratory rate, blood pressure, and orthostatic blood pressure. In some embodiments, blood pressure and pulse measurements will be assessed with a patient in sitting position with a completely automated device. Manual techniques will be used only if an automated device is not available. In some embodiments, blood pressure and pulse measurements should be preceded by at least 5 minutes of rest for the patient in a quiet setting without distractions (e.g., television, cell phones). Vital signs will be taken before blood collection for laboratory tests, if that is scheduled for the same time point.

In some embodiments, improvement, i.e., increase or decrease, in the subject's score on the MMSE, CDR-SB, EQ-5D-5L, NPI, Cognitive Drug Research battery, or any combination thereof, is determined by comparing the subject's score on the MMSE, CDR-SB, EQ-5D-5L, NPI, or Cognitive Drug Research battery, prior to administration of the antibody to the subject's score at one or more timepoints following administration of the antibody and detecting an improved score in the later administered MMSE, CDR-SB, EQ-5D-5L, NPI, or Cognitive Drug Research battery, or any combination thereof. In some embodiments, the subject's score on the MMSE, CDR-SB, EQ-5D-5L, NPI, Cognitive Drug Research battery, or any combination thereof, is determined by comparing the subject's score on the MMSE, CDR-SB, EQ-5D-5L, NPI, or Cognitive Drug Research battery, prior to administration of the antibody to the subject's score at 1, 2, 3, 4, 5, 6, 7, 10, 14, 15, 20, 21, 25, 28, 29, 30, or 31 days following administration of the antibody, or at an interval that is defined by any two of the preceding values. For example, in some embodiments, the subject's score on the MMSE, CDR-SB, EQ-5D-5L, NPI, Cognitive Drug Research battery, or any combination thereof, is determined by comparing the subject's score on the MMSE, CDR-SB, EQ-5D-5L, NPI, or Cognitive Drug Research battery, prior to administration of the antibody to the subject's score at least every 1-31, 1-30, 1-29, 1-28, 1-25, 1-21, 1-15, 1-14, 1-10, 1-7, 1-5, 1-3, 3-31, 3-30, 3-29, 3-28, 3-25, 3-21, 3-15, 3-14, 3-10, 3-7, 3-5, 5-31, 5-30, 5-29, 5-28, 5-25, 5-21, 5-20, 5-15, 5-14, 5-10, 5-7, 7-31, 7-30, 7-29, 7-28, 7-25, 7-21, 7-15, 7-14, 7-10, 10-31, 10-30, 10-29, 10-28, 10-25, 10-21, 10-25, 10-14, 14-31, 14-10, 14-29, 14-28, 14-21, 21-31, 21-30, 21-29, or 21-28 days. In some embodiments, the subject's whole brain volume is measured at least every 1, 2, 3, 4, 5, 6, 7, 8, 9, 10, 11, or 12 months following administration of the antibody, or at an interval that is defined by any two of the preceding values. For example, in some embodiments, the subject's score on the MMSE, CDR-SB, EQ-5D-5L, NPI, Cognitive Drug Research battery, or any combination thereof, is determined by comparing the subject's score on the MMSE, CDR-SB, EQ-5D-5L, NPI, or Cognitive Drug Research battery, prior to administration of the antibody to the subject's score at least every 1-12, 1-9, 1-6, 1-4, 1-3, 3-12, 3-9, 3-6, 6-12, 6-9, or 9-12 months following administration of the antibody. In some embodiments, the subject's score on the MMSE, CDR-SB, EQ-5D-5L, NPI, Cognitive Drug Research battery, or any combination thereof is improved, i.e., increased or decreased, by at least 5, 10, 15, 20, 25, 30, 40, 50, 60, 70, 75, 80, 90, 95, 96, 97, 98, 99, 100, 150, 200, 250, 300, 400, 500, 600, 700, 750, 800, 900 950, or 1000% or by a range that is defined by any two of the preceding values, as compared to the subject's score on one or more previous MMSE, CDR-SB, EQ-5D-5L, NPI, Cognitive Drug Research battery, or any combination thereof. For example, in some embodiments, the subject's score on the MMSE, CDR-SB, EQ-5D-5L, NPI, Cognitive Drug Research battery, or any combination thereof is improved; i.e. increased or decreased, by at least 5-1000, 5-750, 5-500, 5-250, 5-100, 5-95, 5-90, 5-75, 5-50, 5-25, 5-10, 10-100, 10-95, 10-90, 10-75, 10-50, 10-25, 25-100, 25-95, 25-90, 25-75, 25-50, 50-100, 50-95, 50-90, 50-75, 75-100, 75-95, or 75-90%, as compared to the subject's score on one or more previous MMSE, CDR-SB, EQ-5D-5L, NPI, Cognitive Drug Research battery, or any combination thereof. In some embodiments, the subject's score on the MMSE, CDR-SB, EQ-5D-5L, NPI, Cognitive Drug Research battery, or any combination thereof is improved; i.e. increased or decreased, by at least 1-fold, 2-fold, 3-fold, 4-fold, 5-fold, 6-fold, 7-fold, 8-fold, 9-fold, or 10-fold, or by a range that is defined by any two of the preceding values, as compared to the subject's score on one or more previous MMSE, CDR-SB, EQ-5D-5L, NPI, Cognitive Drug Research battery, or any combination thereof. For example, in some embodiments, the subject's score on the MMSE, CDR-SB, EQ-5D-5L, NPI, Cognitive Drug Research battery, or any combination thereof is improved; i.e. increased or decreased, by at least 1-10, 1-7, 1-5, 1-3, 3-10, 3-7, 3-5, 5-10, or 5-7-fold, as compared to the subject's score on one or more previous MMSE, CDR-SB, EQ-5D-5L, NPI, Cognitive Drug Research battery, or any combination thereof.

In some embodiments, decreased amyloid plaque formation is observed in patients treated with anti-Gal3 antibody as compared to amyloid plaque formation measured in the patients prior to administration of one or more doses of the anti-Gal3 antibody. In some embodiments, amyloid plaque formation is measured by PET scan. In some embodiments, one or more PET scans is performed on the subject. In some embodiments, the one or more PET scans are performed at least every 1, 2, 3, 4, 5, 6, 7, 10, 14, 15, 20, 21, 25, 28, 29, 30, or 31 days following administration of the antibody, or at an interval that is defined by any two of the preceding values. For example, in some embodiments, the one or more PET scans are performed at least every 1-31, 1-30, 1-29, 1-28, 1-25, 1-21, 1-15, 1-14, 1-10, 1-7, 1-5, 1-3, 3-31, 3-30, 3-29, 3-28, 3-25, 3-21, 3-15, 3-14, 3-10, 3-7, 3-5, 5-31, 5-30, 5-29, 5-28, 5-25, 5-21, 5-20, 5-15, 5-14, 5-10, 5-7, 7-31, 7-30, 7-29, 7-28, 7-25, 7-21, 7-15, 7-14, 7-10, 10-31, 10-30, 10-29, 10-28, 10-25, 10-21, 10-25, 10-14, 14-31, 14-10, 14-29, 14-28, 14-21, 21-31, 21-30, 21-29, or 21-28 days. In some embodiments, the one or more PET scans are performed at least every 1, 2, 3, 4, 5, 6, 7, 8, 9, 10, 11, or 12 months following administration of the antibody, or at an interval that is defined by any two of the preceding values. For example, in some embodiments, the one or more PET scans are performed at least every 1-12, 1-9, 1-6, 1-4, 1-3, 3-12, 3-9, 3-6, 6-12, 6-9, or 9-12 months following administration of the antibody. In some embodiments, amyloid plaque formation measured following administration of one or more doses of anti-Gal3 antibody is decreased by at least 5, 10, 15, 20, 25, 30, 40, 50, 60, 70, 75, 80, 90, 95, 96, 97, 98, 99, 100, 150, 200, 250, 300, 400, 500, 600, 700, 750, 800, 900 950, or 1000%, or by a range that is defined by any two of the preceding values, as compared to amyloid plaque formation in the subject prior to administration of one or more doses of anti-Gal3 antibody. For example, in some embodiments, amyloid plaque formation measured following administration of one or more doses of anti-Gal3 antibody is decreased by at least 5-1000, 5-75-, 5-500, 5-250, 5-100, 5-95, 5-90, 5-75, 5-50, 5-25, 5-10, 10-100, 10-95, 10-90, 10-75, 10-50, 10-25, 25-100, 25-95, 25-90, 25-75, 25-50, 50-100, 50-95, 50-90, 50-75, 75-100, 75-95, or 75-90%, as compared to amyloid plaque formation in the subject prior to administration of one or more doses of anti-Gal3 antibody. In some embodiments, amyloid plaque formation measured following administration of one or more doses of anti-Gal3 antibody is decreased by at least by at least 1-fold, 2-fold, 3-fold, 4-fold, 5-fold, 6-fold, 7-fold, 8-fold, 9-fold, or 10-fold, or by a range that is defined by any two of the preceding values, as compared to amyloid plaque formation in the subject prior to administration of one or more doses of anti-Gal3 antibody. In some embodiments, amyloid plaque formation measured following administration of one or more doses of anti-Gal3 antibody is decreased by at least by at least 1-10, 1-7, 1-5, 1-3, 3-10, 3-7, 3-5, 5-10, or 5-7-fold, as compared to amyloid plaque formation in the subject prior to administration of one or more doses of anti-Gal3 antibody.

In some embodiments, whole brain volume is increased in a subject following anti-Gal3 antibody administration. In some embodiments, whole brain volume is measured by MRI. In some embodiments, one or more MRI scans is performed on the subject. In some embodiments, the MRIs are performed without the use of ionizing radiation, so patients are not exposed to the harmful effects of ionizing radiation. AEs for MRI scans are very rare. Millions of MRI scans are performed in the US every year, and the FDA receives around 300 AE reports for MRI scanners and coils each year from manufacturers, distributors, user facilities, and patients. The majority of these reports describe heating and/or burns (thermal injuries). To produce good quality images, patients must generally remain very still throughout the entire MRI procedure. In some embodiments, the MRI procedure will be performed by medical staff experienced with the equipment and the technique to minimize any risk to patients. In some embodiments, the one or more MRI scans are performed at least every 1, 2, 3, 4, 5, 6, 7, 10, 14, 15, 20, 21, 25, 28, 29, 30, or 31 days following administration of the antibody, or at an interval that is defined by any two of the preceding values. For example, in some embodiments, the one or more MRI scans are performed at least every 1-31, 1-30, 1-29, 1-28, 1-25, 1-21, 1-15, 1-14, 1-10, 1-7, 1-5, 1-3, 3-31, 3-30, 3-29, 3-28, 3-25, 3-21, 3-15, 3-14, 3-10, 3-7, 3-5, 5-31, 5-30, 5-29, 5-28, 5-25, 5-21, 5-20, 5-15, 5-14, 5-10, 5-7, 7-31, 7-30, 7-29, 7-28, 7-25, 7-21, 7-15, 7-14, 7-10, 10-31, 10-30, 10-29, 10-28, 10-25, 10-21, 10-25, 10-14, 14-31, 14-10, 14-29, 14-28, 14-21, 21-31, 21-30, 21-29, or 21-28 days. In some embodiments, the one or more MRI scans are performed at least every 1, 2, 3, 4, 5, 6, 7, 8, 9, 10, 11, or 12 months following administration of the antibody, or at an interval that is defined by any two of the preceding values. For example, in some embodiments, the one or more MRI scans are performed at least every 1-12, 1-9, 1-6, 1-4, 1-3, 3-12, 3-9, 3-6, 6-12, 6-9, or 9-12 months following administration of the antibody. In some embodiments, the subject's whole brain volume is measured prior to administration of the antibody. In some embodiments, the subject's whole brain volume is measured following administration of the antibody. In some embodiments, the subjects whole brain volume is measured at least every 1, 2, 3, 4, 5, 6, 7, 10, 14, 15, 20, 21, 25, 28, 29, 30, or 31 days following administration of the antibody, or at an interval that is defined by any two of the preceding values. For example, in some embodiments, the subject's whole brain volume is measured every 1-31, 1-30, 1-29, 1-28, 1-25, 1-21, 1-15, 1-14, 1-10, 1-7, 1-5, 1-3, 3-31, 3-30, 3-29, 3-28, 3-25, 3-21, 3-15, 3-14, 3-10, 3-7, 3-5, 5-31, 5-30, 5-29, 5-28, 5-25, 5-21, 5-20, 5-15, 5-14, 5-10, 5-7, 7-31, 7-30, 7-29, 7-28, 7-25, 7-21, 7-15, 7-14, 7-10, 10-31, 10-30, 10-29, 10-28, 10-25, 10-21, 10-25, 10-14, 14-31, 14-10, 14-29, 14-28, 14-21, 21-31, 21-30, 21-29, or 21-28 days. In some embodiments, the subject's whole brain volume is measured at least every 1, 2, 3, 4, 5, 6, 7, 8, 9, 10, 11, or 12 months following administration of the antibody, or at an interval that is defined by any two of the preceding values. For example, in some embodiments, the subject's whole brain volume is measured at least every 1-12, 1-9, 1-6, 1-4, 1-3, 3-12, 3-9, 3-6, 6-12, 6-9, or 9-12 months following administration of the antibody. In some embodiments, whole brain volume measured following administration of one or more doses of anti-Gal3 antibody is increased by at least 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 75, 80, 90, or 100%, or by a range that is defined by any two of the preceding values, as compared to whole brain volume in the subject prior to administration of one or more doses of anti-Gal3 antibody. For example, in some embodiments, whole brain volume measured following administration of one or more doses of anti-Gal3 antibody is increased by at least 1-100, 1-75, 1-50, Jan. 1, 2025, 1-20, 1-15, 1-10, 1-5, 1-3, 3-35, 3-20, 2-15, 3-10, 3-5, 5-100, 5-75, 5-50, 5-25, 5-20, 5-15, 5-10, 10-100, 10-75, 10-50, 10-25, 10-20, 10-15, 15-25, 15-20, 20-25, 25-50, 25-75, 25-100, 50-75, or 50-100%, as compared to whole brain volume in the subject prior to administration of one or more doses of anti-Gal3 antibody.

In some embodiments, the measure of whole brain volume comprises measuring the volume of one or more brain subparts. In some embodiments, the measure of whole brain volume comprises measuring a subject's hippocampal and/or intracranial brain volume. In some embodiments, the measure of whole brain volume comprises measuring a subject's left lateral ventricle volume. In some embodiments, the subject's hippocampal brain volume is measured following administration of the antibody. In some embodiments, the subjects hippocampal brain volume is measured at least every 1, 2, 3, 4, 5, 6, 7, 10, 14, 15, 20, 21, 25, 28, 29, 30, or 31 days following administration of the antibody, or at an interval that is defined by any two of the preceding values. For example, in some embodiments, the subject's hippocampal brain volume is measured every 1-31, 1-30, 1-29, 1-28, 1-25, 1-21, 1-15, 1-14, 1-10, 1-7, 1-5, 1-3, 3-31, 3-30, 3-29, 3-28, 3-25, 3-21, 3-15, 3-14, 3-10, 3-7, 3-5, 5-31, 5-30, 5-29, 5-28, 5-25, 5-21, 5-20, 5-15, 5-14, 5-10, 5-7, 7-31, 7-30, 7-29, 7-28, 7-25, 7-21, 7-15, 7-14, 7-10, 10-31, 10-30, 10-29, 10-28, 10-25, 10-21, 10-25, 10-14, 14-31, 14-10, 14-29, 14-28, 14-21, 21-31, 21-30, 21-29, or 21-28 days. In some embodiments, the subject's hippocampal brain volume is measured at least every 1, 2, 3, 4, 5, 6, 7, 8, 9, 10, 11, or 12 months following administration of the antibody, or at an interval that is defined by any two of the preceding values. For example, in some embodiments, the subject's hippocampal brain volume is measured at least every 1-12, 1-9, 1-6, 1-4, 1-3, 3-12, 3-9, 3-6, 6-12, 6-9, or 9-12 months following administration of the antibody. In some embodiments, hippocampal brain volume measured following administration of one or more doses of anti-Gal3 antibody is increased by at least 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 75, 80, 90, or 100%, or by a range that is defined by any two of the preceding values, as compared to hippocampal volume in the subject prior to administration of one or more doses of anti-Gal3 antibody. For example, in some embodiments, hippocampal brain volume measured following administration of one or more doses of anti-Gal3 antibody is increased by at least 1-100, 1-75, 1-50, Jan. 1, 2025, 1-20, 1-15, 1-10, 1-5, 1-3, 3-35, 3-20, 2-15, 3-10, 3-5, 5-100, 5-75, 5-50, 5-25, 5-20, 5-15, 5-10, 10-100, 10-75, 10-50, 10-25, 10-20, 10-15, 15-25, 15-20, 20-25, 25-50, 25-75, 25-100, 50-75, or 50-100%, as compared to hippocampal brain volume in the subject prior to administration of one or more doses of anti-Gal3 antibody.

In some embodiments, hippocampal brain volume increases by at least about 0.01, 0.02, 0.03, 0.04, 0.05, 0.06, 0.07, 0.08, 0.09, 0.10, 0.12, 0.13, 0.14, 0.15, 0.16, 0.17, 0.18, 0.19, 0.20, 0.21, 0.22, 0.23, 0.24, or 0.25 mL, or by a range that is defined by any two of the preceding values, as compared to hippocampal brain volume in the subject prior to administration of one or more doses of anti-Gal3 antibody. For example, in some embodiments, hippocampal brain volume increases by at least about 0.01-0.25, 0.01-0.2, 0.01-0.15, 0.01-0.1, 0.01-0.08, 0.01-0.06, 0.01-0.04, 0.01-0.02, 0.02-0.25, 0.02-0.2, 0.02-0.15, 0.02-0.1, 0.02-0.08, 0.02-0.06, 0.02-0.04, 0.04-0.25, 0.04-0.2, 0.04-0.15, 0.04-0.1, 0.04-0.08, 0.04-0.06, 0.06-0.25, 0.06-0.2, 0.06-0.15, 0.06-0.1, 0.06-0.08, 0.08-0.25, 0.08-0.2, 0.08-0.15, 0.08-0.1, 0.1-0.25, 0.1-0.2, 0.1-0.15, 0.15-0.25, 0.15-0.2, or 0.2-0.25 mL, as compared to hippocampal brain volume in the subject prior to administration of one or more doses of anti-Gal3 antibody. In some embodiments, hippocampal brain volume increases by at least about 0.01, 0.02, 0.03, 0.04, 0.05, 0.06, 0.07, 0.08, 0.09, 0.10, 0.12, 0.13, 0.14, 0.15, 0.16, 0.17, 0.18, 0.19, 0.20, 0.21, 0.22, 0.23, 0.24, or 0.25 mL, or by a range that is defined by any two of the preceding values, within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody. For example, in some embodiments, the subject's hippocampal brain volume increases by at least about 0.01-0.25, 0.01-0.2, 0.01-0.15, 0.01-0.1, 0.01-0.08, 0.01-0.06, 0.01-0.04, 0.01-0.02, 0.02-0.25, 0.02-0.2, 0.02-0.15, 0.02-0.1, 0.02-0.08, 0.02-0.06, 0.02-0.04, 0.04-0.25, 0.04-0.2, 0.04-0.15, 0.04-0.1, 0.04-0.08, 0.04-0.06, 0.06-0.25, 0.06-0.2, 0.06-0.15, 0.06-0.1, 0.06-0.08, 0.08-0.25, 0.08-0.2, 0.08-0.15, 0.08-0.1, 0.1-0.25, 0.1-0.2, 0.1-0.15, 0.15-0.25, 0.15-0.2, or 0.2-0.25 mL, within about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody.

In some embodiments, hippocampal brain volume in subject's treated with TB006 increases by at least about 0.02-0.08 mL. In some embodiments, hippocampal brain volume in subject's treated with TB006 increases by at least about 0.02-0.08 mL within at least about 104 days following administration of the TB006.

In some embodiments, the subject's hippocampal brain volume increases remains increased for at least 1, 2, 3, 4, 5, 6, 7, 8, 9, 10, 11, or 12 months, or for a time period that is defined by any two of the preceding values, following administration of the antibody. For example, in some embodiments, the subject's increased hippocampal brain volume increases remains increased for at least 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12 months following administration of the antibody. In some embodiments, the subject's hippocampal brain volume increases remains increased for greater than 12 months. In some embodiments, the subject's hippocampal brain volume increases within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody and remains increased for at least about 1, 2, 3, 4, 5, 6, 7, 8, 9, 10, 11, or 12 months, or for a time period that is defined by any two of the preceding values, following administration of the antibody. For example, in some embodiments, the subject's hippocampal brain volume increases within at least about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody and remains increased for at least about 1, 2, 3, 4, 5, 6, 7, 8, 9, 10, 11, or 12 months, or for a time period that is defined by any two of the preceding values, following administration of the antibody.

In some embodiments, the subject's intracranial brain volume is measured following administration of the antibody. In some embodiments, the subject's intracranial brain volume is measured at least every 1, 2, 3, 4, 5, 6, 7, 10, 14, 15, 20, 21, 25, 28, 29, 30, or 31 days following administration of the antibody, or at an interval that is defined by any two of the preceding values. For example, in some embodiments, the subject's intracranial brain volume is measured every 1-31, 1-30, 1-29, 1-28, 1-25, 1-21, 1-15, 1-14, 1-10, 1-7, 1-5, 1-3, 3-31, 3-30, 3-29, 3-28, 3-25, 3-21, 3-15, 3-14, 3-10, 3-7, 3-5, 5-31, 5-30, 5-29, 5-28, 5-25, 5-21, 5-20, 5-15, 5-14, 5-10, 5-7, 7-31, 7-30, 7-29, 7-28, 7-25, 7-21, 7-15, 7-14, 7-10, 10-31, 10-30, 10-29, 10-28, 10-25, 10-21, 10-25, 10-14, 14-31, 14-10, 14-29, 14-28, 14-21, 21-31, 21-30, 21-29, or 21-28 days. In some embodiments, the subject's intracranial brain volume is measured at least every 1, 2, 3, 4, 5, 6, 7, 8, 9, 10, 11, or 12 months following administration of the antibody, or at an interval that is defined by any two of the preceding values. For example, in some embodiments, the subject's intracranial brain volume is measured at least every 1-12, 1-9, 1-6, 1-4, 1-3, 3-12, 3-9, 3-6, 6-12, 6-9, or 9-12 months following administration of the antibody. In some embodiments, intracranial brain volume measured following administration of one or more doses of anti-Gal3 antibody is decreased by at least 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 75, 80, 90, or 100%, or by a range that is defined by any two of the preceding values, as compared to intracranial volume in the subject prior to administration of one or more doses of anti-Gal3 antibody. For example, in some embodiments, intracranial brain volume measured following administration of one or more doses of anti-Gal3 antibody is decreased by at least 1-100, 1-75, 1-50, Jan. 1, 2025, 1-20, 1-15, 1-10, 1-5, 1-3, 3-35, 3-20, 2-15, 3-10, 3-5, 5-100, 5-75, 5-50, 5-25, 5-20, 5-15, 5-10, 10-100, 10-75, 10-50, 10-25, 10-20, 10-15, 15-25, 15-20, 20-25, 25-50, 25-75, 25-100, 50-75, or 50-100%, as compared to intracranial brain volume in the subject prior to administration of one or more doses of anti-Gal3 antibody.

In some embodiments, intercranial brain volume decreases by at least about 1, 5, 10, 15, 20, 25, 30, 35, 40, 45, 50, 55, 60, 65, 70, 75, 80, 85, 90, 95, or 100 mL, or by a range that is defined by any two of the preceding values, as compared to intracranial brain volume in the subject prior to administration of one or more doses of anti-Gal3 antibody. For example, in some embodiments, intercranial brain volume decreases by at least about 1-100, 1-80, 1-75, 1-60, 1-50, 1-40, 1-25, 1-20, 1-10, 10-100, 10-80, 10-75, 10-60, 10-50, 10-40, 10-25, 10-20, 20-100, 20-80, 20-75, 20-60, 20-50, 20-40, 20-25, 25-100, 25-80, 25-75, 25-60, 25-50, 25-40, 40-100, 40-80, 40-75, 40-60, 40-50, 50-100, 50-80, 50-75, 50-60, 60-100, 60-80, 60-75, 75-100, 75-80, or 80-100 mL, as compared to intracranial brain volume in the subject prior to administration of one or more doses of anti-Gal3 antibody. In some embodiments, intercranial brain volume decreases by at least about 1, 5, 10, 15, 20, 25, 30, 35, 40, 45, 50, 55, 60, 65, 70, 75, 80, 85, 90, 95, or 100 mL, or by a range that is defined by any two of the preceding values, within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody. For example, in some embodiments, intercranial brain volume decreases by at least about 1-100, 1-80, 1-75, 1-60, 1-50, 1-40, 1-25, 1-20, 1-10, 10-100, 10-80, 10-75, 10-60, 10-50, 10-40, 10-25, 10-20, 20-100, 20-80, 20-75, 20-60, 20-50, 20-40, 20-25, 25-100, 25-80, 25-75, 25-60, 25-50, 25-40, 40-100, 40-80, 40-75, 40-60, 40-50, 50-100, 50-80, 50-75, 50-60, 60-100, 60-80, 60-75, 75-100, 75-80, or 80-100 mL, within about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody.

In some embodiments, intracranial brain volume in subject's treated with TB006 decreases by at least about 20 mL. In some embodiments, intracranial brain volume in subject's treated with TB006 decreases by at least about 20 mL within at least about 104 days following administration of the TB006.

In some embodiments, the subject's intercranial brain volume remains decreased for at least 1, 2, 3, 4, 5, 6, 7, 8, 9, 10, 11, or 12 months, or for a time period that is defined by any two of the preceding values, following administration of the antibody. For example, in some embodiments, the subject's decreased intercranial brain volume remains decreased for at least 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12 months following administration of the antibody. In some embodiments, the subject's intercranial brain volume remains decreased for greater than 12 months. In some embodiments, the subject's intercranial brain volume decreases within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody and remains decreased for at least about 1, 2, 3, 4, 5, 6, 7, 8, 9, 10, 11, or 12 months, or for a time period that is defined by any two of the preceding values, following administration of the antibody. For example, in some embodiments, the subject's intercranial brain volume decreases within at least about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody and remains decreased for at least about 1, 2, 3, 4, 5, 6, 7, 8, 9, 10, 11, or 12 months, or for a time period that is defined by any two of the preceding values, following administration of the antibody.

In some embodiments, the subject's left lateral ventricle volume is measured following administration of the antibody. In some embodiments, the subject's left lateral ventricle volume is measured at least every 1, 2, 3, 4, 5, 6, 7, 10, 14, 15, 20, 21, 25, 28, 29, 30, or 31 days following administration of the antibody, or at an interval that is defined by any two of the preceding values. For example, in some embodiments, the subject's left lateral ventricle volume is measured every 1-31, 1-30, 1-29, 1-28, 1-25, 1-21, 1-15, 1-14, 1-10, 1-7, 1-5, 1-3, 3-31, 3-30, 3-29, 3-28, 3-25, 3-21, 3-15, 3-14, 3-10, 3-7, 3-5, 5-31, 5-30, 5-29, 5-28, 5-25, 5-21, 5-20, 5-15, 5-14, 5-10, 5-7, 7-31, 7-30, 7-29, 7-28, 7-25, 7-21, 7-15, 7-14, 7-10, 10-31, 10-30, 10-29, 10-28, 10-25, 10-21, 10-25, 10-14, 14-31, 14-10, 14-29, 14-28, 14-21, 21-31, 21-30, 21-29, or 21-28 days. In some embodiments, the subject's left lateral ventricle volume is measured at least every 1, 2, 3, 4, 5, 6, 7, 8, 9, 10, 11, or 12 months following administration of the antibody, or at an interval that is defined by any two of the preceding values. For example, in some embodiments, the subject's left lateral ventricle volume is measured at least every 1-12, 1-9, 1-6, 1-4, 1-3, 3-12, 3-9, 3-6, 6-12, 6-9, or 9-12 months following administration of the antibody. In some embodiments, left lateral ventricle volume is measured following administration of one or more doses of anti-Gal3 antibody is decreased by at least 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 75, 80, 90, or 100%, or by a range that is defined by any two of the preceding values, as compared to intracranial volume in the subject prior to administration of one or more doses of anti-Gal3 antibody. For example, in some embodiments, left lateral ventricle volume measured following administration of one or more doses of anti-Gal3 antibody is decreased by at least 1-100, 1-75, 1-50, Jan. 1, 2025, 1-20, 1-15, 1-10, 1-5, 1-3, 3-35, 3-20, 2-15, 3-10, 3-5, 5-100, 5-75, 5-50, 5-25, 5-20, 5-15, 5-10, 10-100, 10-75, 10-50, 10-25, 10-20, 10-15, 15-25, 15-20, 20-25, 25-50, 25-75, 25-100, 50-75, or 50-100%, as compared to intracranial brain volume in the subject prior to administration of one or more doses of anti-Gal3 antibody.

In some embodiments, left lateral ventricle volume decreases by at least about 1, 2, 3, 4, 5, 6, 7, 8, 9, or 10 mL, or by a range that is defined by any two of the preceding values, as compared to left lateral ventricle volume in the subject prior to administration of one or more doses of anti-Gal3 antibody. In some embodiments, left lateral ventricle volume decreases by at least about 1, 2, 3, 4, 5, 6, 7, 8, 9, or 10 mL, or by a range that is defined by any two of the preceding values, within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody.

In some embodiments, the subject's left lateral ventricle volume remains decreased for at least 1, 2, 3, 4, 5, 6, 7, 8, 9, 10, 11, or 12 months, or for a time period that is defined by any two of the preceding values, following administration of the antibody. For example, in some embodiments, the subject's decreased left lateral ventricle volume remains decreased for at least 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12 months following administration of the antibody. In some embodiments, the subject's left lateral ventricle volume remains decreased for greater than 12 months. In some embodiments, the subject's left lateral ventricle volume decreases within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody and remains decreased for at least about 1, 2, 3, 4, 5, 6, 7, 8, 9, 10, 11, or 12 months, or for a time period that is defined by any two of the preceding values, following administration of the antibody.

In some embodiments, the subjects left lateral ventricle volume increases by at least about 1, 2, 3, 4, 5, 6, 7, 8, 9, or 10 mL, or by a range that is defined by any two of the preceding values, as compared to left lateral ventricle volume in the subject prior to administration of one or more doses of anti-Gal3 antibody. In some embodiments, left lateral ventricle volume increases by at least about 1, 2, 3, 4, 5, 6, 7, 8, 9, or 10 mL, or by a range that is defined by any two of the preceding values, within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody.

In some embodiments, the CDR-SB test comprises one or more tests of the subject's memory, orientation, judgement and problem solving, community affairs, home and hobbies, personal care, or any combination thereof. In some embodiments, the CDR-SB test is performed at least every 1, 2, 3, 4, 5, 6, 7, 10, 14, 15, 20, 21, 25, 28, 29, 30, or 31 days following administration of the antibody, or at an interval that is defined by any two of the preceding values. For example, in some embodiments, the CDR-SB test is performed at least every 1-31, 1-30, 1-29, 1-28, 1-25, 1-21, 1-15, 1-14, 1-10, 1-7, 1-5, 1-3, 3-31, 3-30, 3-29, 3-28, 3-25, 3-21, 3-15, 3-14, 3-10, 3-7, 3-5, 5-31, 5-30, 5-29, 5-28, 5-25, 5-21, 5-20, 5-15, 5-14, 5-10, 5-7, 7-31, 7-30, 7-29, 7-28, 7-25, 7-21, 7-15, 7-14, 7-10, 10-31, 10-30, 10-29, 10-28, 10-25, 10-21, 10-25, 10-14, 14-31, 14-10, 14-29, 14-28, 14-21, 21-31, 21-30, 21-29, or 21-28 days. In some embodiments, the CDR-SB test is performed at least every 1, 2, 3, 4, 5, 6, 7, 8, 9, 10, 11, or 12 months following administration of the antibody, or at an interval that is defined by any two of the preceding values. For example, in some embodiments, the CDR-SB test is performed at least every 1-12, 1-9, 1-6, 1-4, 1-3, 3-12, 3-9, 3-6, 6-12, 6-9, or 9-12 months following administration of the antibody.

In some embodiments, the subject's global CDR-SB score is decreased by at least 0.5, 1, 1.5, 2, 2.5, 3, 3.5, 4, 4.5, 5, 6, 7, 8, or nine points, or by a range that is defined by any two of the preceding values. For example, in some embodiments, the subject's global CDR-SB score is decreased by at least 0.5-9, 0.5-7, 0.5-5, 0.5-3, 3-9, 3-7, 3-5, 5-9, 5-7, or 7-9 points.

In some embodiments, the subject's global CDR-SB score is decreased by at least 1 point. In some embodiments, the subject's global CDR-SB score is decreased by at least 1 point within at least about 104 days following administration of the antibody. In some embodiments, the subject's global CDR-SB score is decreased by at least about 0.23 points from baseline within about 15 days of administration of the antibody. In some embodiments, the subject's global CDR-SB score is decreased by at least about 0.34 points from baseline within about 36 days of administration of the antibody. In some embodiments, the subject's global CDR-SB score is decreased by at least about 0.27 points from baseline within about 64 days of administration of the antibody. In some embodiments, the subject's global CDR-SB score is decreased by at least about 0.43 points from baseline within about 104 days of administration of the antibody. In some embodiments, the subject's global CDR-SB score is decreased by at least about 0.56 points from baseline within about 13 weeks of administration of the antibody. In some embodiments, the subject's global CDR-SB score is decreased by at least about 0.56 points from baseline following about 13 weeks of once-monthly administration of the antibody. In some embodiments, the subject's global CDR-SB score is decreased by at least about 0.01, 0.03, 0.06, 0.09, 0.1, 0.12, 0.15, 0.2, 0.25, 0.3, 0.4, 0.5, 0.51, 0.52, 0.53, 0.54, 0.55, 0.56, 0.57, 0.58, 0.59, 0.6, 0.7, 0.75, 0.8, 0.9, or 1 points, from baseline following about 13 weeks of once-monthly administration of the antibody, or by a range that is defined by any two of the preceding values. For example, in some embodiments, the subject's global CDR-SB score is decreased by between about 0.01-1, 0.01-0.75, 0.01-0.56, 0.01-0.5, 0.01-0.25, 0.01-0.12, 0.01-0.1, 0.1-1, 0.1-0.75, 0.1-0.56, 0.1-0.5, 0.1-0.25, 0.25-1, 0.25-0.75, 0.25-0.5, 0.5-1, 0.5-0.75, or 0.75-1, point.

In some embodiments, the subject's global CDR-SB score is decreased by at least 5, 10, 15, 20, 25, 30, 40, 50, 60, 70, 75, 80, 90, 95, 96, 97, 98, 99, 100, 150, 200, 250, 300, 400, 500, 600, 700, 750, 800, 900 950, or 1000%, or by a range that is defined by any two of the preceding values. For example, in some embodiments, the subject's global CDR-SB score is decreased by at least 5-1000, 5-750, 5-500, 5-250, 5-100, 5-95, 5-90, 5-75, 5-50, 5-25, 5-10, 10-100, 10-95, 10-90, 10-75, 10-50, 10-25, 25-100, 25-95, 25-90, 25-75, 25-50, 50-100, 50-95, 50-90, 50-75, 75-100, 75-95, or 75-90%. In some embodiments, the subject's global CDR-SB score is decreased by at least 1-fold, 2-fold, 3-fold, 4-fold, 5-fold, 6-fold, 7-fold, 8-fold, 9-fold, or 10-fold, or by a range that is defined by any two of the preceding values. For example, in some embodiments, the subject's global CDR-SB score is decreased by at least 1-10, 1-7, 1-5, 1-3, 3-10, 3-7, 3-5, 5-10, or 5-7-fold.

In some embodiments, the subject's mean global CDR-SB score is decreased by at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 points, or by a range that is defined by any two of the preceding values, as compared to the subject's mean global CDR-SB score prior to administration of the antibody. For example, in some embodiments, the subject's mean global CDR-SB score is decreased by at least 0.1-3, 0.1-2.5, 0.1-2, 0.1-1.5, 0.1-1, 0.1-0.75, 0.1-0.5, 0.1-0.25, 0.25-3, 0.25-2.5, 0.25-2, 0.25-1.5, 0.25-1, 0.25-0.75, 0.25-0.5, 0.5-3, 0.5-2.5, 0.5-2, 0.5-1.5, 0.5-1, 1-3, 1-2.5, 1-2, 1-1.5, 1.5-3, 1.5-2.5, 1.5-2, 2-3, 2-2.5, or 2.5-3 points, as compared to the subject's mean global CDR-SB score prior to administration of the antibody. In some embodiments, the subject's mean global CDR-SB score is decreased by at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 points, or by a range that is defined by any two of the preceding values, within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody. For example, in some embodiments, the subject's mean global CDR-SB score is decreased by at least 0.1-3, 0.1-2.5, 0.1-2, 0.1-1.5, 0.1-1, 0.1-0.75, 0.1-0.5, 0.1-0.25, 0.25-3, 0.25-2.5, 0.25-2, 0.25-1.5, 0.25-1, 0.25-0.75, 0.25-0.5, 0.5-3, 0.5-2.5, 0.5-2, 0.5-1.5, 0.5-1, 1-3, 1-2.5, 1-2, 1-1.5, 1.5-3, 1.5-2.5, 1.5-2, 2-3, 2-2.5, or 2.5-3 points, within about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody.

In some embodiments, the subject's mean global CDR-SB score is decreased by at least 0.1-1 point following administration of the antibody. In some embodiments, the subject's mean global CDR-SB score is decreased by at least 0.1-1 point within at least 104 days following administration of the antibody.

In some embodiments, the subject's global CDR score remains decreased for at least 1, 2, 3, 4, 5, 6, 7, 8, 9, 10, 11, or 12 months, or for a time period that is defined by any two of the preceding values, following administration of the antibody. For example, in some embodiments, the subject's decreased global CDR score remains decreased for at least 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12 months following administration of the antibody. In some embodiments, the subject's global CDR score remains decreased for greater than 12 months. In some embodiments, the subject's global CDR score decreases within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody and remains decreased for at least about 1, 2, 3, 4, 5, 6, 7, 8, 9, 10, 11, or 12 months, or for a time period that is defined by any two of the preceding values, following administration of the antibody. For example, in some embodiments, the subject's global CDR score decreases within at least about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody and remains decreased for at least about 1, 2, 3, 4, 5, 6, 7, 8, 9, 10, 11, or 12 months, or for a time period that is defined by any two of the preceding values, following administration of the antibody.

In some embodiments, the subject's score on tests of memory, orientation, judgement and problem solving, community affairs, home and hobbies, and/or personal care score on the CDR-SB, are decreased by at least 0.5, 1, 2, or 3 points, or by a range that is defined by any two of the preceding values. For example, in some embodiments, the subject's score on tests of memory, orientation, judgement and problem solving, community affairs, home and hobbies, and/or personal care score on the CDR-SB, are decreased by at least 0.5-3, 0.5-2, 0.5-1, 1-3, 1-2, or 2-3 points.

In some embodiments, the subject's Memory CDR-SB subdomain score is decreased by at least 1, 2, 3, 4, 5, 10, 15, 20, 25, 30, 40, 50, 60, 70, 75, 80, 90, 95, 96, 97, 98, 99, 100, 150, 200, 250, 300, 400, 500, 600, 700, 750, 800, 900 950, or 1000%, or by a range that is defined by any two of the preceding values. For example, in some embodiments, the subject's Memory CDR-SB subdomain score is decreased by at least 1-1000, 1-500, 1-250, 1-100, 1-75, 1-50, 1-25, 1-10, 1-5, 5-1000, 5-750, 5-500, 5-250, 5-100, 5-95, 5-90, 5-75, 5-50, 5-25, 5-10, 10-100, 10-95, 10-90, 10-75, 10-50, 10-25, 25-100, 25-95, 25-90, 25-75, 25-50, 50-100, 50-95, 50-90, 50-75, 75-100, 75-95, or 75-90%. In some embodiments, the subject's Memory CDR-SB subdomain score is decreased by at least 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, or 10-fold, or by a range that is defined by any two of the preceding values. For example, in some embodiments, the subject's Memory CDR-SB subdomain score is decreased by at least 0.1-10, 0.1-7, 0.1-5, 0.1-3, 0.1-1, 0.1-0.7, 0.1-0.5, 0.1-0.3, 0.3-10, 0.3-7, 0.3-5, 0.3-3, 0.3-1, 0.3-0.7, 0.3-0.5, 0.5-10, 0.5-7, 0.5-5, 0.5-3, 0.5-1, 0.5-0.7, 1-10, 1-7, 1-5, 1-3, 3-10, 3-7, 3-5, 5-10, or 5-7-fold.

In some embodiments, the subject's mean Memory CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5, or by a range that is defined by any two of the preceding values, as compared to the subject's mean Memory CDR-SB subdomain score prior to administration of the antibody. For example, in some embodiments, the subject's mean Memory CDR-SB subdomain score is decreased by at least 0.01-0.5, 0.01-0.25, 0.01-0.2, 0.01-0.15, 0.01-0.1, 0.01-0.05, 0.01-0.025, 0.01-0.1, 0.01-0.05, 0.05-0.5, 0.05-0.25, 0.05-0.02, 0.05-0.15, 0.05-0.1, 0.1-0.5, 0.1-0.25, 0.1-0.2, 0.1-0.15, 0.15-0.5, 0.15-0.25, 0.15-0.2, 0.2-0.5, 0.2-0.25, or 0.25-0.5 points, as compared to the subject's mean subject's mean Memory CDR-SB subdomain score prior to administration of the antibody. In some embodiments, the subject's mean Memory CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5 points, or by a range that is defined by any two of the preceding values, within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody. For example, in some embodiments, the subject's mean Memory CDR-SB subdomain score is decreased by at least 0.01-0.5, 0.01-0.25, 0.01-0.2, 0.01-0.15, 0.01-0.1, 0.01-0.05, 0.01-0.025, 0.01-0.1, 0.01-0.05, 0.05-0.5, 0.05-0.25, 0.05-0.02, 0.05-0.15, 0.05-0.1, 0.1-0.5, 0.1-0.25, 0.1-0.2, 0.1-0.15, 0.15-0.5, 0.15-0.25, 0.15-0.2, 0.2-0.5, 0.2-0.25, or 0.25-0.5 points, within about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody.

In some embodiments, the subject's Memory CDR-SB subdomain score is decreased by at least 0.05-0.3 points following administration of the antibody. In some embodiments, the subject's Memory CDR-SB subdomain score is decreased by at least 0.05-0.3 points within at least 104 days following administration of the antibody. In some embodiments, the subject's Memory CDR-SB subdomain score is decreased by at least 0.05-0.3 points within at least 64 days following administration of the antibody.

In some embodiments, the subject's decreased Memory CDR-Subdomain score remains decreased for at least 1, 2, 3, 4, 5, 6, 7, 8, 9, 10, 11, or 12 months, or for a time period that is defined by any two of the preceding values, following administration of the antibody. For example, in some embodiments, the subject's decreased Memory CDR-Subdomain score remains decreased for at least 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12 months following administration of the antibody. In some embodiments, the subject's Memory CDR-Subdomain score remains decreased for greater than 12 months. In some embodiments, the subject's Memory CDR-Subdomain score decreases within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody and remains decreased for at least about 1, 2, 3, 4, 5, 6, 7, 8, 9, 10, 11, or 12 months, or for a time period that is defined by any two of the preceding values, following administration of the antibody. For example, in some embodiments, the subject's Memory CDR-Subdomain score decreases within at least about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody and remains decreased for at least about 1, 2, 3, 4, 5, 6, 7, 8, 9, 10, 11, or 12 months, or for a time period that is defined by any two of the preceding values, following administration of the antibody.

In some embodiments, the subject's Orientation CDR-SB subdomain score is decreased by at least 1, 2, 3, 4, 5, 10, 15, 20, 25, 30, 40, 50, 60, 70, 75, 80, 90, 95, 96, 97, 98, 99, 100, 150, 200, 250, 300, 400, 500, 600, 700, 750, 800, 900 950, or 1000%, or by a range that is defined by any two of the preceding values. For example, in some embodiments, the subject's Orientation CDR-SB subdomain score is decreased by at least 1-1000, 1-500, 1-250, 1-100, 1-75, 1-50, 1-25, 1-10, 1-5, 5-1000, 5-750, 5-500, 5-250, 5-100, 5-95, 5-90, 5-75, 5-50, 5-25, 5-10, 10-100, 10-95, 10-90, 10-75, 10-50, 10-25, 25-100, 25-95, 25-90, 25-75, 25-50, 50-100, 50-95, 50-90, 50-75, 75-100, 75-95, or 75-90%. In some embodiments, the subject's Orientation CDR-SB subdomain score is decreased by at least 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, or 10-fold, or by a range that is defined by any two of the preceding values. For example, in some embodiments, the subject's Orientation CDR-SB subdomain score is decreased by at least 0.1-10, 0.1-7, 0.1-5, 0.1-3, 0.1-1, 0.1-0.7, 0.1-0.5, 0.1-0.3, 0.3-10, 0.3-7, 0.3-5, 0.3-3, 0.3-1, 0.3-0.7, 0.3-0.5, 0.5-10, 0.5-7, 0.5-5, 0.5-3, 0.5-1, 0.5-0.7, 1-10, 1-7, 1-5, 1-3, 3-10, 3-7, 3-5, 5-10, or 5-7-fold.

In some embodiments, the subject's mean Orientation CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5, or by a range that is defined by any two of the preceding values, as compared to the subject's mean Orientation CDR-SB subdomain score prior to administration of the antibody. For example, in some embodiments, the subject's mean Orientation CDR-SB subdomain score is decreased by at least 0.01-0.5, 0.01-0.3, 0.01-0.25, 0.01-0.2, 0.01-0.15, 0.01-0.1, 0.01-0.05, 0.01-0.025, 0.01-0.1, 0.01-0.05, 0.05-0.5, 00.05-0.3.05-0.25, 0.05-0.02, 0.05-0.15, 0.05-0.1, 0.1-0.5, 0.1-0.3, 0.1-0.25, 0.1-0.2, 0.1-0.15, 0.15-0.5, 0.15-0.25, 0.15-0.2, 0.2-0.5, 0.2-0.25, 0.25-0.5, or 0.3-0.5 points, as compared to the subject's mean subject's mean Orientation CDR-SB subdomain score prior to administration of the antibody. In some embodiments, the subject's mean Orientation CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5 points, or by a range that is defined by any two of the preceding values, within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of TB006. For example, in some embodiments, the subject's mean Orientation CDR-Subdomain score is decreased by at least 0.01-0.5, 0.01-0.3, 0.01-0.25, 0.01-0.2, 0.01-0.15, 0.01-0.1, 0.01-0.05, 0.01-0.025, 0.01-0.1, 0.01-0.05, 0.05-0.5, 00.05-0.3.05-0.25, 0.05-0.02, 0.05-0.15, 0.05-0.1, 0.1-0.5, 0.1-0.3, 0.1-0.25, 0.1-0.2, 0.1-0.15, 0.15-0.5, 0.15-0.25, 0.15-0.2, 0.2-0.5, 0.2-0.25, 0.25-0.5, or 0.3-0.5 points, within about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody.

In some embodiments, the subject's Orientation CDR-SB subdomain score is decreased by at least 0.05-0.3 points following administration of the antibody. In some embodiments, the subject's Orientation CDR-SB subdomain score is decreased by at least 0.05-0.3 points within at least 104 days following administration of the antibody. In some embodiments, the subject's Orientation CDR-SB subdomain score is decreased by at least 0.05-0.3 points within at least about 15 days following administration of the antibody. In some embodiments, the subject's Orientation CDR-SB subdomain score is decreased by at least 0.05-0.3 points within at least about 36 days following administration of the antibody.

In some embodiments, the subject's decreased Orientation CDR-Subdomain score remains decreased for at least 1, 2, 3, 4, 5, 6, 7, 8, 9, 10, 11, or 12 months, or for a time period that is defined by any two of the preceding values, following administration of the antibody. For example, in some embodiments, the subject's decreased Orientation CDR-Subdomain score remains decreased for at least 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12 months following administration of the antibody. In some embodiments, the subject's Orientation CDR-Subdomain score remains decreased for greater than 12 months. In some embodiments, the subject's Orientation CDR-Subdomain score decreases within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody and remains decreased for at least about 1, 2, 3, 4, 5, 6, 7, 8, 9, 10, 11, or 12 months, or for a time period that is defined by any two of the preceding values, following administration of the antibody. For example, in some embodiments, the subject's Orientation CDR-Subdomain score decreases within at least about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody and remains decreased for at least about 1, 2, 3, 4, 5, 6, 7, 8, 9, 10, 11, or 12 months, or for a time period that is defined by any two of the preceding values, following administration of the antibody.

In some embodiments, the subject's Judgement and Problem-Solving CDR-SB subdomain score is decreased by at least 1, 2, 3, 4, 5, 10, 15, 20, 25, 30, 40, 50, 60, 70, 75, 80, 90, 95, 96, 97, 98, 99, 100, 150, 200, 250, 300, 400, 500, 600, 700, 750, 800, 900 950, or 1000%, or by a range that is defined by any two of the preceding values. For example, in some embodiments, the subject's Judgement and Problem-Solving CDR-SB subdomain score is decreased by at least 1-1000, 1-500, 1-250, 1-100, 1-75, 1-50, 1-25, 1-10, 1-5, 5-1000, 5-750, 5-500, 5-250, 5-100, 5-95, 5-90, 5-75, 5-50, 5-25, 5-10, 10-100, 10-95, 10-90, 10-75, 10-50, 10-25, 25-100, 25-95, 25-90, 25-75, 25-50, 50-100, 50-95, 50-90, 50-75, 75-100, 75-95, or 75-90%. In some embodiments, the subject's Judgement and Problem-Solving CDR-SB subdomain score is decreased by at least 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, or 10-fold, or by a range that is defined by any two of the preceding values. For example, in some embodiments, the subject's Judgement and Problem-Solving CDR-SB subdomain score is decreased by at least 0.1-10, 0.1-7, 0.1-5, 0.1-3, 0.1-1, 0.1-0.7, 0.1-0.5, 0.1-0.3, 0.3-10, 0.3-7, 0.3-5, 0.3-3, 0.3-1, 0.3-0.7, 0.3-0.5, 0.5-10, 0.5-7, 0.5-5, 0.5-3, 0.5-1, 0.5-0.7, 1-10, 1-7, 1-5, 1-3, 3-10, 3-7, 3-5, 5-10, or 5-7-fold.

In some embodiments, the subject's mean Judgement and Problem-Solving CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5, or by a range that is defined by any two of the preceding values, as compared to the subject's mean Judgement and Problem-Solving CDR-SB subdomain score prior to administration of the antibody. For example, in some embodiments, the subject's mean Judgement and Problem-Solving CDR-SB subdomain score is decreased by at least 0.01-0.5, 0.01-0.3, 0.01-0.25, 0.01-0.2, 0.01-0.15, 0.01-0.1, 0.01-0.05, 0.01-0.025, 0.01-0.1, 0.01-0.05, 0.05-0.5, 00.05-0.3.05-0.25, 0.05-0.02, 0.05-0.15, 0.05-0.1, 0.1-0.5, 0.1-0.3, 0.1-0.25, 0.1-0.2, 0.1-0.15, 0.15-0.5, 0.15-0.25, 0.15-0.2, 0.2-0.5, 0.2-0.25, 0.25-0.5, or 0.3-0.5 points, as compared to the subject's mean subject's mean Judgement and Problem-Solving CDR-SB subdomain score prior to administration of the antibody. In some embodiments, the subject's mean Judgement and Problem-Solving CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5 points, or by a range that is defined by any two of the preceding values, within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody. For example, in some embodiments, the subject's mean Judgement and Problem-Solving CDR-Subdomain score is decreased by at least 0.01-0.5, 0.01-0.3, 0.01-0.25, 0.01-0.2, 0.01-0.15, 0.01-0.1, 0.01-0.05, 0.01-0.025, 0.01-0.1, 0.01-0.05, 0.05-0.5, 00.05-0.3.05-0.25, 0.05-0.02, 0.05-0.15, 0.05-0.1, 0.1-0.5, 0.1-0.3, 0.1-0.25, 0.1-0.2, 0.1-0.15, 0.15-0.5, 0.15-0.25, 0.15-0.2, 0.2-0.5, 0.2-0.25, 0.25-0.5, or 0.3-0.5 points, within about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody.

In some embodiments, the subject's Judgement and Problem-Solving CDR-SB subdomain score is decreased by at least 0.05-0.3 points following administration of the antibody. In some embodiments, the subject's Judgement and Problem-Solving CDR-SB subdomain score is decreased by at least 0.05-0.3 points within at least 104 days following administration of the antibody.

In some embodiments, the subject's decreased Judgement and Problem-Solving CDR-Subdomain score remains decreased for at least 1, 2, 3, 4, 5, 6, 7, 8, 9, 10, 11, or 12 months, or for a time period that is defined by any two of the preceding values, following administration of the antibody. For example, in some embodiments, the subject's decreased Judgement and Problem-Solving CDR-Subdomain score remains decreased for at least 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12 months following administration of the antibody. In some embodiments, the subject's Judgement and Problem-Solving CDR-Subdomain score remains decreased for greater than 12 months. In some embodiments, the subject's Judgement and Problem-Solving CDR-Subdomain score decreases within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody and remains decreased for at least about 1, 2, 3, 4, 5, 6, 7, 8, 9, 10, 11, or 12 months, or for a time period that is defined by any two of the preceding values, following administration of the antibody. For example, in some embodiments, the subject's Judgement and Problem-Solving CDR-Subdomain score decreases within at least about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody and remains decreased for at least about 1, 2, 3, 4, 5, 6, 7, 8, 9, 10, 11, or 12 months, or for a time period that is defined by any two of the preceding values, following administration of the antibody.

In some embodiments, the subject's Community Affairs CDR-SB subdomain score is decreased by at least 1, 2, 3, 4, 5, 10, 15, 20, 25, 30, 40, 50, 60, 70, 75, 80, 90, 95, 96, 97, 98, 99, 100, 150, 200, 250, 300, 400, 500, 600, 700, 750, 800, 900 950, or 1000%, or by a range that is defined by any two of the preceding values. For example, in some embodiments, the subject's Community Affairs CDR-SB subdomain score is decreased by at least 1-1000, 1-500, 1-250, 1-100, 1-75, 1-50, 1-25, 1-10, 1-5, 5-1000, 5-750, 5-500, 5-250, 5-100, 5-95, 5-90, 5-75, 5-50, 5-25, 5-10, 10-100, 10-95, 10-90, 10-75, 10-50, 10-25, 25-100, 25-95, 25-90, 25-75, 25-50, 50-100, 50-95, 50-90, 50-75, 75-100, 75-95, or 75-90%. In some embodiments, the subject's Community Affairs CDR-SB subdomain score is decreased by at least 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, or 10-fold, or by a range that is defined by any two of the preceding values. For example, in some embodiments, the subject's Community Affairs CDR-SB subdomain score is decreased by at least 0.1-10, 0.1-7, 0.1-5, 0.1-3, 0.1-1, 0.1-0.7, 0.1-0.5, 0.1-0.3, 0.3-10, 0.3-7, 0.3-5, 0.3-3, 0.3-1, 0.3-0.7, 0.3-0.5, 0.5-10, 0.5-7, 0.5-5, 0.5-3, 0.5-1, 0.5-0.7, 1-10, 1-7, 1-5, 1-3, 3-10, 3-7, 3-5, 5-10, or 5-7-fold.

In some embodiments, the subject's mean Community Affairs CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5, or by a range that is defined by any two of the preceding values, as compared to the subject's mean Community Affairs CDR-SB subdomain score prior to administration of the antibody. For example, in some embodiments, the subject's mean Community Affairs CDR-SB subdomain score is decreased by at least 0.01-0.5, 0.01-0.3, 0.01-0.25, 0.01-0.2, 0.01-0.15, 0.01-0.1, 0.01-0.05, 0.01-0.025, 0.01-0.1, 0.01-0.05, 0.05-0.5, 00.05-0.3, 0.05-0.25, 0.05-0.02, 0.05-0.15, 0.05-0.1, 0.1-0.5, 0.1-0.3, 0.1-0.25, 0.1-0.2, 0.1-0.15, 0.15-0.5, 0.15-0.25, 0.15-0.2, 0.2-0.5, 0.2-0.25, 0.25-0.5, or 0.3-0.5 points, as compared to the subject's mean subject's mean Community Affairs CDR-SB subdomain score prior to administration of the antibody. In some embodiments, the subject's mean Community Affairs CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5 points, or by a range that is defined by any two of the preceding values, within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody. For example, in some embodiments, the subject's mean Community Affairs CDR-Subdomain score is decreased by at least 0.01-0.5, 0.01-0.3, 0.01-0.25, 0.01-0.2, 0.01-0.15, 0.01-0.1, 0.01-0.05, 0.01-0.025, 0.01-0.1, 0.01-0.05, 0.05-0.5, 00.05-0.3.05-0.25, 0.05-0.02, 0.05-0.15, 0.05-0.1, 0.1-0.5, 0.1-0.3, 0.1-0.25, 0.1-0.2, 0.1-0.15, 0.15-0.5, 0.15-0.25, 0.15-0.2, 0.2-0.5, 0.2-0.25, 0.25-0.5, or 0.3-0.5 points, within about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody.

In some embodiments, the subject's Community Affairs CDR-SB subdomain score is decreased by at least 0.05-0.3 points following administration of the antibody. In some embodiments, the subject's Community Affairs CDR-SB subdomain score is decreased by at least 0.05-0.3 points within at least 104 days following administration of the antibody. In some embodiments, the subject's Community Affairs CDR-SB subdomain score is decreased by at least 0.05-0.3 points within at least 36 days following administration of the antibody.

In some embodiments, the subject's decreased Community Affairs CDR-Subdomain score remains decreased for at least 1, 2, 3, 4, 5, 6, 7, 8, 9, 10, 11, or 12 months, or for a time period that is defined by any two of the preceding values, following administration of the antibody. For example, in some embodiments, the subject's decreased Community Affairs CDR-Subdomain score remains decreased for at least 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12 months following administration of the antibody. In some embodiments, the subject's decreased Community Affairs CDR-Subdomain score remains decreased for greater than 12 months. In some embodiments, the subject's Community Affairs CDR-Subdomain score decreases within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody and remains increased for at least about 1, 2, 3, 4, 5, 6, 7, 8, 9, 10, 11, or 12 months, or for a time period that is defined by any two of the preceding values, following administration of the antibody. For example, in some embodiments, the subject's Community Affairs CDR-Subdomain score decreases within at least about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody and remains decreased for at least about 1, 2, 3, 4, 5, 6, 7, 8, 9, 10, 11, or 12 months, or for a time period that is defined by any two of the preceding values, following administration of the antibody.

In some embodiments, the subject's Home and Hobbies CDR-SB subdomain score is decreased by at least 1, 2, 3, 4, 5, 10, 15, 20, 25, 30, 40, 50, 60, 70, 75, 80, 90, 95, 96, 97, 98, 99, 100, 150, 200, 250, 300, 400, 500, 600, 700, 750, 800, 900 950, or 1000%, or by a range that is defined by any two of the preceding values. For example, in some embodiments, the subject's Home and Hobbies CDR-SB subdomain score is decreased by at least 1-1000, 1-500, 1-250, 1-100, 1-75, 1-50, 1-25, 1-10, 1-5, 5-1000, 5-750, 5-500, 5-250, 5-100, 5-95, 5-90, 5-75, 5-50, 5-25, 5-10, 10-100, 10-95, 10-90, 10-75, 10-50, 10-25, 25-100, 25-95, 25-90, 25-75, 25-50, 50-100, 50-95, 50-90, 50-75, 75-100, 75-95, or 75-90%. In some embodiments, the subject's Home and Hobbies CDR-SB subdomain score is decreased by at least 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, or 10-fold, or by a range that is defined by any two of the preceding values. For example, in some embodiments, the subject's Home and Hobbies CDR-SB subdomain score is decreased by at least 0.1-10, 0.1-7, 0.1-5, 0.1-3, 0.1-1, 0.1-0.7, 0.1-0.5, 0.1-0.3, 0.3-10, 0.3-7, 0.3-5, 0.3-3, 0.3-1, 0.3-0.7, 0.3-0.5, 0.5-10, 0.5-7, 0.5-5, 0.5-3, 0.5-1, 0.5-0.7, 1-10, 1-7, 1-5, 1-3, 3-10, 3-7, 3-5, 5-10, or 5-7-fold.

In some embodiments, the subject's mean Home and Hobbies CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5, or by a range that is defined by any two of the preceding values, as compared to the subject's mean Home and Hobbies CDR-SB subdomain score prior to administration of the antibody. For example, in some embodiments, the subject's mean Home and Hobbies CDR-SB subdomain score is decreased by at least 0.01-0.5, 0.01-0.3, 0.01-0.25, 0.01-0.2, 0.01-0.15, 0.01-0.1, 0.01-0.05, 0.01-0.025, 0.01-0.1, 0.01-0.05, 0.05-0.5, 00.05-0.3, 0.05-0.25, 0.05-0.02, 0.05-0.15, 0.05-0.1, 0.1-0.5, 0.1-0.3, 0.1-0.25, 0.1-0.2, 0.1-0.15, 0.15-0.5, 0.15-0.25, 0.15-0.2, 0.2-0.5, 0.2-0.25, 0.25-0.5, or 0.3-0.5 points, as compared to the subject's mean Home and Hobbies CDR-SB subdomain score prior to administration of the antibody. In some embodiments, the subject's mean Home and Hobbies CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5 points, or by a range that is defined by any two of the preceding values, within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody. For example, in some embodiments, the subject's mean Home and Hobbies CDR-Subdomain score is decreased by at least 0.01-0.5, 0.01-0.3, 0.01-0.25, 0.01-0.2, 0.01-0.15, 0.01-0.1, 0.01-0.05, 0.01-0.025, 0.01-0.1, 0.01-0.05, 0.05-0.5, 00.05-0.3, 0.05-0.25, 0.05-0.02, 0.05-0.15, 0.05-0.1, 0.1-0.5, 0.1-0.3, 0.1-0.25, 0.1-0.2, 0.1-0.15, 0.15-0.5, 0.15-0.25, 0.15-0.2, 0.2-0.5, 0.2-0.25, 0.25-0.5, or 0.3-0.5 points, within about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody.

In some embodiments, the subject's Home and Hobbies CDR-SB subdomain score is decreased by at least 0.05-0.3 points following administration of the antibody. In some embodiments, the subject's Home and Hobbies CDR-SB subdomain score is decreased by at least 0.05-0.3 points within at least 104 days following administration of the antibody.

In some embodiments, the subject's decreased Home and Hobbies CDR-Subdomain score remains decreased for at least 1, 2, 3, 4, 5, 6, 7, 8, 9, 10, 11, or 12 months, or for a time period that is defined by any two of the preceding values, following administration of the antibody. For example, in some embodiments, the subject's decreased Home and Hobbies CDR-Subdomain score remains decreased for at least 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12 months following administration of the antibody. In some embodiments, the subject's decreased Home and Hobbies CDR-Subdomain score remains decreased for greater than 12 months. In some embodiments, the subject's Home and Hobbies CDR-Subdomain score decreases within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody and remains increased for at least about 1, 2, 3, 4, 5, 6, 7, 8, 9, 10, 11, or 12 months, or for a time period that is defined by any two of the preceding values, following administration of the antibody. For example, in some embodiments, the subject's Home and Hobbies CDR-Subdomain score decreases within at least about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody and remains decreased for at least about 1, 2, 3, 4, 5, 6, 7, 8, 9, 10, 11, or 12 months, or for a time period that is defined by any two of the preceding values, following administration of the antibody.

In some embodiments, the subject's Personal Care CDR-SB subdomain score is decreased by at least 1, 2, 3, 4, 5, 10, 15, 20, 25, 30, 40, 50, 60, 70, 75, 80, 90, 95, 96, 97, 98, 99, 100, 150, 200, 250, 300, 400, 500, 600, 700, 750, 800, 900 950, or 1000%, or by a range that is defined by any two of the preceding values. For example, in some embodiments, the subject's Personal Care CDR-SB subdomain score is decreased by at least 1-1000, 1-500, 1-250, 1-100, 1-75, 1-50, 1-25, 1-10, 1-5, 5-1000, 5-750, 5-500, 5-250, 5-100, 5-95, 5-90, 5-75, 5-50, 5-25, 5-10, 10-100, 10-95, 10-90, 10-75, 10-50, 10-25, 25-100, 25-95, 25-90, 25-75, 25-50, 50-100, 50-95, 50-90, 50-75, 75-100, 75-95, or 75-90%. In some embodiments, the subject's Personal Care CDR-SB subdomain CDR-SB subdomain score is decreased by at least 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, or 10-fold, or by a range that is defined by any two of the preceding values. For example, in some embodiments, the subject's Personal Care CDR-SB subdomain score is decreased by at least 0.1-10, 0.1-7, 0.1-5, 0.1-3, 0.1-1, 0.1-0.7, 0.1-0.5, 0.1-0.3, 0.3-10, 0.3-7, 0.3-5, 0.3-3, 0.3-1, 0.3-0.7, 0.3-0.5, 0.5-10, 0.5-7, 0.5-5, 0.5-3, 0.5-1, 0.5-0.7, 1-10, 1-7, 1-5, 1-3, 3-10, 3-7, 3-5, 5-10, or 5-7-fold.

In some embodiments, the subject's mean Personal Care CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5, or by a range that is defined by any two of the preceding values, as compared to the subject's mean Personal Care CDR-SB subdomain score prior to administration of the antibody. For example, in some embodiments, the subject's mean Personal Care CDR-SB subdomain score is decreased by at least 0.01-0.5, 0.01-0.3, 0.01-0.25, 0.01-0.2, 0.01-0.15, 0.01-0.1, 0.01-0.05, 0.01-0.025, 0.01-0.1, 0.01-0.05, 0.05-0.5, 00.05-0.3, 0.05-0.25, 0.05-0.02, 0.05-0.15, 0.05-0.1, 0.1-0.5, 0.1-0.3, 0.1-0.25, 0.1-0.2, 0.1-0.15, 0.15-0.5, 0.15-0.25, 0.15-0.2, 0.2-0.5, 0.2-0.25, 0.25-0.5, or 0.3-0.5 points, as compared to the subject's mean subject's mean Personal Care CDR-SB subdomain score prior to administration of the antibody. In some embodiments, the subject's mean Personal Care CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5 points, or by a range that is defined by any two of the preceding values, within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody. For example, in some embodiments, the subject's mean Personal Care CDR-Subdomain score is decreased by at least 0.01-0.5, 0.01-0.3, 0.01-0.25, 0.01-0.2, 0.01-0.15, 0.01-0.1, 0.01-0.05, 0.01-0.025, 0.01-0.1, 0.01-0.05, 0.05-0.5, 00.05-0.3, 0.05-0.25, 0.05-0.02, 0.05-0.15, 0.05-0.1, 0.1-0.5, 0.1-0.3, 0.1-0.25, 0.1-0.2, 0.1-0.15, 0.15-0.5, 0.15-0.25, 0.15-0.2, 0.2-0.5, 0.2-0.25, 0.25-0.5, or 0.3-0.5 points, within about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody.

In some embodiments, the subject's Personal Care CDR-SB subdomain score is decreased by at least 0.05-0.3 points following administration of the antibody. In some embodiments, the subject's Personal Care CDR-SB subdomain score is decreased by at least 0.05-0.3 points within at least 104 days following administration of the antibody.

In some embodiments, the subject's decreased Personal Care CDR-Subdomain score remains decreased for at least 1, 2, 3, 4, 5, 6, 7, 8, 9, 10, 11, or 12 months, or for a time period that is defined by any two of the preceding values, following administration of the antibody. For example, in some embodiments, the subject's decreased Personal Care CDR-Subdomain score remains decreased for at least 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12 months following administration of the antibody. In some embodiments, the subject's decreased Personal Care CDR-Subdomain score remains decreased for greater than 12 months. In some embodiments, the subject's Personal Care CDR-Subdomain score decreases within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody and remains increased for at least about 1, 2, 3, 4, 5, 6, 7, 8, 9, 10, 11, or 12 months, or for a time period that is defined by any two of the preceding values, following administration of the antibody. For example, in some embodiments, the subject's Personal Care CDR-Subdomain score decreases within at least about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody and remains decreased for at least about 1, 2, 3, 4, 5, 6, 7, 8, 9, 10, 11, or 12 months, or for a time period that is defined by any two of the preceding values, following administration of the antibody.

In some embodiments, the MMSE test comprises one or more tests of the subject's orientation to time, orientation to place, registration, attention and calculation, recall, naming, repetition, comprehension, reading, writing, and/or drawing, or any combination thereof.

In some embodiments, the subject's MMSE score is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 points, or by a range that is defined by any two of the preceding values. For example, in some embodiments, the subject's MMSE score is increased by at least 1-30, 1-29, 1-25, 1-20, 1-15, 1-10, 1-5, 1-3, 3-30, 3-29, 3-25, 3-20, 3-15, 3-10, 3-7, 3-5, 5-30, 5-29, 5-25, 5-20, 5-15, 5-10, 5-7, 7-30, 7-29, 7-25, 7-20, 7-15, 7-10, 10-30, 10-29, 10-25, 10-20, 10-15, 15-30, 15-29, 15-25, 15-20, 20-30, 20-29, 20-25, 25-30, or 25-29 points.

In some embodiments, the subject's orientation to time, orientation to place, and/or attention and calculation score on the MMSE, are increased by at least 1, 2, 3, 4, or 5 points, or by a range that is defined by any two of the preceding values. For example, in some embodiments the subject's orientation to time, orientation to place, and/or attention and calculation score on the MMSE, are increased by at least 1-5, 1-4, 1-3, 1-2, 2-5, 2-4, or 2-3 points.

In some embodiments, the subject's registration, recall, or comprehension score on the MMSE is increased by at least 1, 2, or 3 points, or by a range that is defined by any two of the preceding values. For example, in some embodiments, the subject's registration, recall, or comprehension score on the MMSE is increased by at least 1-3, 1-2, or 2-3 points. In some embodiments, the subject's repetition, reading, writing, or drawing score on the MMSE is increased by at least 1 point.

In some embodiments, the MMSE score is increased by at least 5, 10, 15, 20, 25, 30, 40, 50, 60, 70, 75, 80, 90, 95, 96, 97, 98, 99, 100, 150, 200, 250, 300, 400, 500, 600, 700, 750, 800, 900 950, or 1000%, or by a range that is defined by any two of the preceding values.

In some embodiments, the subject's MMSE score is increased by at least 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, or 10-fold, or by a range that is defined by any two of the preceding values. For example, in some embodiments, the subject's MMSE score is increased by at least 0.1-10, 0.1-7, 0.1-5, 0.1-3, 0.1-1, 0.1-0.7, 0.1-0.5, 0.1-0.3, 0.3-10, 0.3-7, 0.3-5, 0.3-3, 0.3-1, 0.3-0.7, 0.3-0.5, 0.5-10, 0.5-7, 0.5-5, 0.5-3, 0.5-1, 0.5-0.7, 1-10, 1-7, 1-5, 1-3, 3-10, 3-7, 3-5, 5-10, or 5-7-fold.

In some embodiments, the subject's mean MMSE score is increased by at least 1, 2, 3, 4, 5, 6, 7, 8, 9, or 10 points, or by a range that is defined by any two of the preceding values, as compared to the subject's mean MMSE score prior to administration of the antibody. For example, in some embodiments, the subject's mean MMSE score is increases by at least 1-10, 1-7, 1-5, 1-3, 3-10, 3-7, 3-5, 5-10, 5-7, or 7-10 points, as compared to the subject's mean MMSE score prior to administration of the antibody. In some embodiments, the subject's mean MMSE score is increased by at least 1, 2, 3, 4, 5, 6, 7, 8, 9, or 10 points, or by a range that is defined by any two of the preceding values, within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody. For example, in some embodiments, the subject's mean MMSE score is increased by at least 1, 2, 3, 4, 5, 6, 7, 8, 9, or 10 points, or by a range that is defined by any two of the preceding values, within about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody.

In some embodiments, the subject's increased MMSE score remains increased for at least 1, 2, 3, 4, 5, 6, 7, 8, 9, 10, 11, or 12 months, or for a time period that is defined by any two of the preceding values, following administration of the antibody. For example, in some embodiments, the subject's increased MMSE score remains increased for at least 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12 months following administration of the antibody. In some embodiments, the subject's increased MMSE score remains increased for greater than 12 months. In some embodiments, the subject's MMSE score increases within at least about 1, 5, 7, 8, 10, 14, 15, 21, 28, 29, 30, 31, 36, 59, 60, 61, 64, 89, 90, 91, 100, 104, or 105 days, or a time frame that is defined by any two of the preceding values, following administration of the antibody and remains increased for at least about 1, 2, 3, 4, 5, 6, 7, 8, 9, 10, 11, or 12 months, or for a time period that is defined by any two of the preceding values, following administration of the antibody. For example, in some embodiments, the subject's MMSE score increases within at least about 1-105, 1-104, 1-100, 1-90, 1-66, 1-60, 1-36, 1-30, 1-15, 1-8, 8-105, 1-104, 8-90, 8-66, 8-60, 8-30, 8-15, 15-105, 15-104, 15-90, 15-66, 15-60, 15-34, 15-30, 30-105, 30-104, 30-90, 30-66, 30-34, 34-105, 34-104, 34-90, 34-66, 34-60, 60-105, 60-104, 60-90, 60-66, 66-105, 66-104, 66-90, 90-105, 90-104, or 104-105 days following administration of the antibody and remains increased for at least about 1, 2, 3, 4, 5, 6, 7, 8, 9, 10, 11, or 12 months, or for a time period that is defined by any two of the preceding values, following administration of the antibody.

In some embodiments, the one or more inflammation biomarkers comprise lymphocytes, white blood cells, C-reactive protein (CRP/hsCRP), interleukins, IL6, tumor necrosis factor alpha (TNFα), monocytes, interleukins, IL1, IL2, IL3, IL4, IL5, IL6, IL7, IL8, IL9, IL10, IL11, IL12, IL13, IL14, IL15, IL16, IL17, IL18, IL19, IL20, IL21, IL22, IL23, IL24, IL25, IL26, IL27, IL28, IL29, IL30, IL31, IL32, IL33, IL34, IL35, IL36, IL37, IL38, IL39, IL 40, IL1-β, transforming growth factor beta (TGF-β), SIRT1, CXCL1, or any combination thereof.

In some embodiments, the one or more biomarkers comprise amyloid beta peptide 40, amyloid beta peptide 42, pTau181, GFAP, NFL, Gal3, and/or any combination thereof.

In some embodiments, the one or more diabetes biomarkers comprise insulin, glucose, fasted glucose, glycated hemoglobin (Hba1c), C-peptide, adiponectin, SIRT 6, or any combination thereof. In some embodiments, the one or more cancer biomarkers comprise TNFα, extracellular vesicles (EVs), autophagy, TGF-β, SIRT1, plasminogen activator inhibitor-1 (PAI1), CXCL1, or any combination thereof. In some embodiments, the one or more cardiovascular disease biomarkers comprise CRP, hsCRP, cholesterol, LDL-cholesterol, triglycerides, HDL-cholesterol, atherosclerosis, atherosclerotic legions, blood pressure, systolic blood pressure, or any combination thereof. In some embodiments, the one or more kidney damage or dysfunction biomarkers comprise albumin, creatinine, GDF15, cystatin c, urea, or any combination thereof. In some embodiments, the one or more liver damage and/or disfunction biomarkers comprise albumin, alkaline phosphatase, bilirubin, adiponectin, GDF15, or any combination thereof. In some embodiments, the one or more anemia biomarkers comprise hemoglobin, hematocrit, RBC, mean corpuscular volume (MCV), mean corpuscular hemoglobin concentration (MCHC), or any combination thereof. In some embodiments, the one or more physical capability biomarkers comprise grip strength, walking speed, standing balance, a timed up and go test, or any combination thereof. In some embodiments, the one or more organ function biomarkers comprise atherosclerotic lesions, muscle mass, systolic blood pressure, cognitive function, body mass index, bone density, lung function, waist circumference, or any combination thereof. In some embodiments, the one or more biomarkers are measured within at least 1, 2, 3, 4, 5, 6, 7, 8, 9, 10, 11, 12, 13, or 14 days after antibody administration, or at an interval that is defined by any two of the preceding values. For example, in some embodiments, the one or more biomarkers are measured within at least 1-14, 1-10, 1-7, 1-5, 1-3, 3-14, 3-10, 3-7, 3-5, 5-14, 5-10, 5-7, 7-14, 7-10, or 10-14 days following antibody administration. In some embodiments, the one or more biomarkers are measured within at least 1, 2, 3, 4, 5, 6, 7, 8, 9, 10, 11, or 12 months following administration of the antibody, or at an interval that is defined by any two of the preceding values. For example, in some embodiments, the one or more biomarkers are measured within at least 1-12, 1-9, 1-6, 1-4, 1-3, 3-12, 3-9, 3-6, 6-12, 6-9, or 9-12 months following administration of the antibody.

In some embodiments, the antibody is at a dose of at least 70, 75, 80, 90, 100, 110, 120, 125, 130, 140, 150, 160, 170, 180, 190, 200, 250, 300, 350, 360, 370, 380, 390, 400, 410, 420, 430, 440, 450, 500, 600, 700, 800, 900, 950, 960, 970, 980, 990, 1000, 1010, 1020, 1025, 1030, 1040, 1050, 1100, 1200, 1300, 1400, 1500, 2000, 2100, 2200, 2300, 2400, 2500, 2600, 2700, 2750, 2800, 3900, 3000, 3500, 66,00, 7300, 3750, 3800, 3900, 4000, 5000, or 7500 mg, or at a range that is defined by any two of the preceding values. For example, in some embodiments, the antibody is at a dose of at least 70-7500, 70-5000, 70-4000, 70-3000, 70-2500, 70-2000, 70-1500, 70-1000, 70-500, 70-250, 250-7500, 250-5000, 250-4000, 250-3000, 250-2500, 250-2000, 250-1500, 250-1000, 250-750, 250-500, 500-7500, 500-5000, 500-4000, 500-3000, 500-2500, 500-2000, 500-1500, 500-1000, 500-750, 1000-7500, 1000-5000, 1000-4000, 1000-3000, 1000-2500, 1000-2000, 1000-1500, 2500-7500, 2500-5000, or 5000-7500 mg. In some embodiments, the anti-Gal3 antibody is at a dose of at least 70, 140, or 1000 mg, or at a range that is defined by any two of the preceding values.

In some embodiments, the antibody is administered at a dose of at least 0.1, 0.5, 1, 2, 3, 4, 5, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 175, 200, 225, 250, 275, or 300 mg/kg, or by a range that is defined by any two of the preceding values. For example, in some embodiments, the antibody is administered at a dose of at least 0.1-300, 0.1-200, 0.1-100, 0.1-50, 0.1-25, 25-300, 25-200, 25-100, 25-50, 50-300, 50-200, 50-100, 100-300, 100-200, or 200-300 mg/kg. In some embodiments, the antibody is administered at a dose of at least 0.1, 2, 4, 6, 10, 14, 15, 20, 25, 30, 40, 50, 60, 70 mg/kg, or by a range that is defined by any two of the preceding values. For example, in some embodiments, the antibody is administered at a dose of at least 0.1-70, 0.1-50, 0.1-25, 0.1-15, 0.1-10, 0.1-6, 0.1-4, 4-70, 4-50, 4-25, 4-15, 4-14, 4-6, 6-70, 6-50, 6-25, 6-15, 6-14, 14-70, 14-50, 14-25, 25-70, or 25-50 mg/kg. In some embodiments, the antibody is administered at a dose of at least 2, 6, or 14 mg/kg, or by a range that is defined by any two of the preceding values. For example, in some embodiments, the antibody is administered at a dose of at least 2-14, 2-6, or 6-14 mg/kg.

In some embodiments, the antibody is administered over the course of at least 10, 20, 30, 40, 50, 60, 70, 80, 90, 100, 120, 130, 140, 150, 160, 170, 180, 190, or 200 minutes, or by a range that is defined by any two of the preceding values. For example, in some embodiments, the antibody is administered over the course of at least 10-200, 10-180, 10-150, 10-120, 10-90, 10-60, 10-30, 10-20, 30-200, 30-180, 30-150, 30-120, 30-90, 30-60, 60-200, 60-180, 60-120, 60-90, 90-200, 90-180, or 90-120 minutes.

In some embodiments, the antibody is formulated at 20 mg/ml. In some embodiments, the 20 mg/ml can be diluted to 8 mg/ml in a total of 500 mL prior to I. V. administration.

In some embodiments, the method comprises diluting the antibody prior to administration, wherein the antibody is diluted in sterile normal saline for injection. In some embodiments, the method comprises diluting a 20 mg/mL (in some embodiments, the 20 mg/ml can be diluted to 8 mg/ml in a total of 500 mL prior to I. V. administration) antibody formulation in an IV bag containing 0.9% sodium chloride and normal saline. In some embodiments, the antibody diluted in sterile normal saline is administered to the subject by IV infusion over one hour. In some embodiments, the antibody diluted in sterile saline is administered to the subjects at least once every 28, 29, 30, or 31 days.

In some embodiments, the antibody is administered over the course of at least 60 minutes. In some embodiments, the antibody is administered at least every 2 days, 3 days, 4 days, 5 days, 6 days, 7 days, 10 days, 12 days, 15 days, 20 days, or 28 days, 29 days, 30 days, 31 days, or by a range that is defined by any two of the preceding values. In some embodiments, the antibody is administered at least every 28, 29, 30, and/or 31 days. In some embodiments, the antibody is administered at least 1, 2, 3, 4, 5, 6, 7, 8, 9, 10, 11, 12 times, or at an interval that is defined by any two of the preceding values.

In some embodiments, the antibody is administered at least 1, 2, 3, 4, 5, 6, 7, 8, 9, or 10 times per 28 day cycle, or at an interval that is defined by any two of the preceding values. For example, in some embodiments, the antibody is administered at least 1-10, 1-7, 1-5, 1-3, 3-10, 3-7, 3-5, 5-10, or 5-7 times per 28, 29, 30, or 31 day cycle. In some embodiments, the antibody is administered more than 10 times per 28, 29, 30, or 31 day cycle. In some embodiments, the antibody is administered at least once per every 28, 29, 30, or 31 day cycle for up to 113 weeks. In some embodiments, the antibody is administered at least once per every 28, 29, 30, or 31 day cycle indefinitely. In some embodiments, the antibody is administered at least daily, weekly, bi-weekly, every 10 days, monthly, bi-monthly, semi-annually, or even annually.

In some embodiments, the antibody is administered monthly at a dose of 5000 mg.

In some embodiments, the method comprises administration of one or more pharmaceuticals. In some embodiments, the one or more pharmaceuticals comprises anti-infectives, antibiotics, corticosteroids, opioid analgesics, anxiolytics, muscle relaxants, paracetamol, acetaminophen, or any combination thereof.

In some embodiments, one or more physical exams is administered on the subject.

In some embodiments, the pharmacokinetics of the antibody in the subject's blood is evaluated following antibody administration.

In some embodiments, the subject is at least 35, 40, 45, 50, 55, 60, 65, 60, 75, 80, 85, or 90 years old, or is at an age that is defined by any two of the preceding values. For example, in some embodiments, the subject is at least 35-90, 35-80, 35-75, 35-70, 35-65, 35-60, 35-50, 35-40, 40-90, 40-80, 40-75, 40-60, 40-50, 50-90, 50-75, or 50-60 years old.

In some embodiments, the antibody comprises (1) a heavy chain variable region comprising a VH-CDR1, a VH-CDR2, and a VH-CDR3; and (2) a light chain variable region comprising a VL-CDR1, a VL-CDR2, and a VL-CDR3, wherein the VH-CDR1 comprises an amino acid sequence having at least 90% identity to SEQ ID NO: 31; the VH-CDR2 comprises an amino acid sequence having at least 90% identity to SEQ ID NO: 72; the VH-CDR3 comprises an amino acid sequence having at least 90% identity to SEQ ID NO: 113; the VH-CDR1 comprises an amino acid sequence having at least 90% identity to SEQ ID NO: 170; the VH-CDR1 comprises an amino acid sequence having at least 90% identity to SEQ ID NO: 221; and the VH-CDR1 comprises an amino acid sequence having at least 90%, to SEQ ID NO: 248.

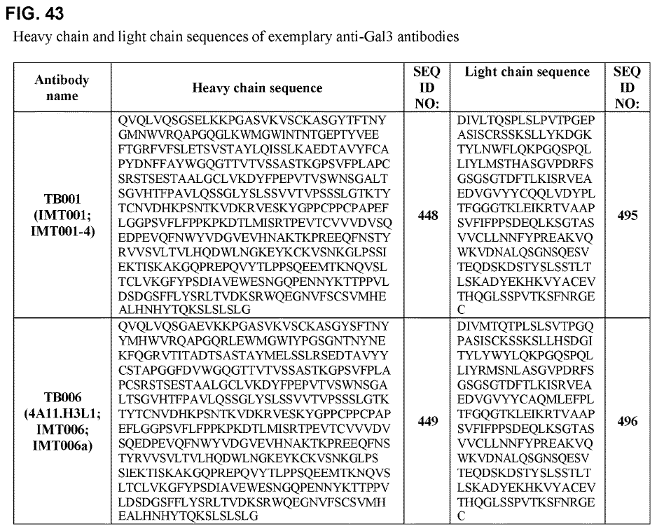

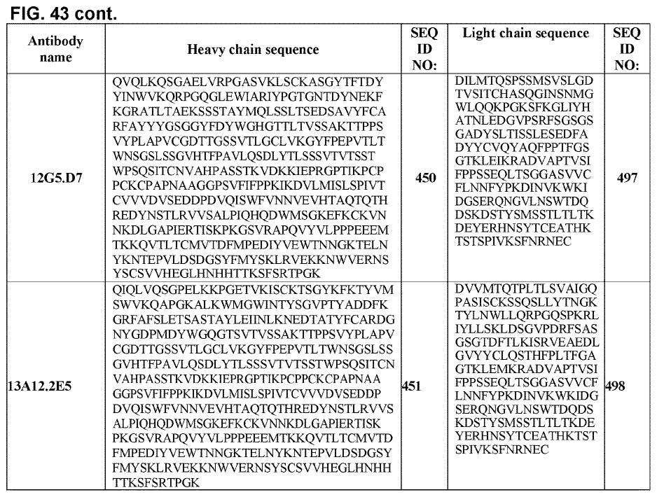

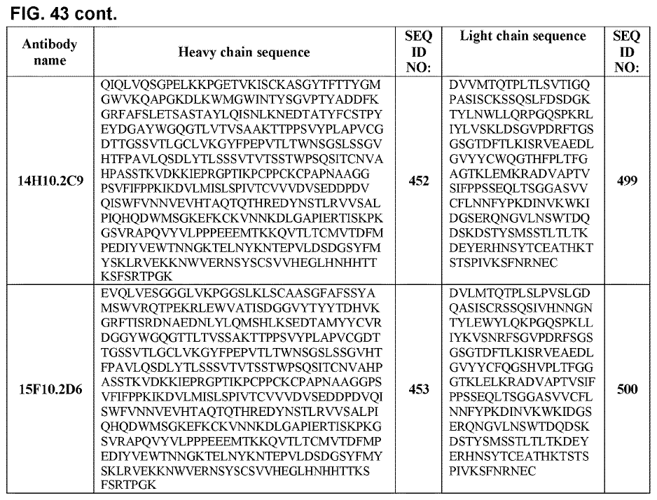

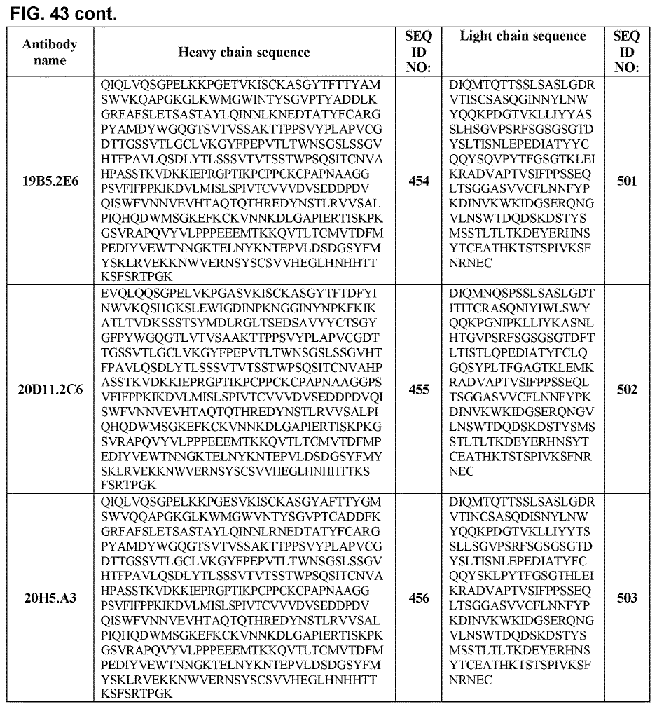

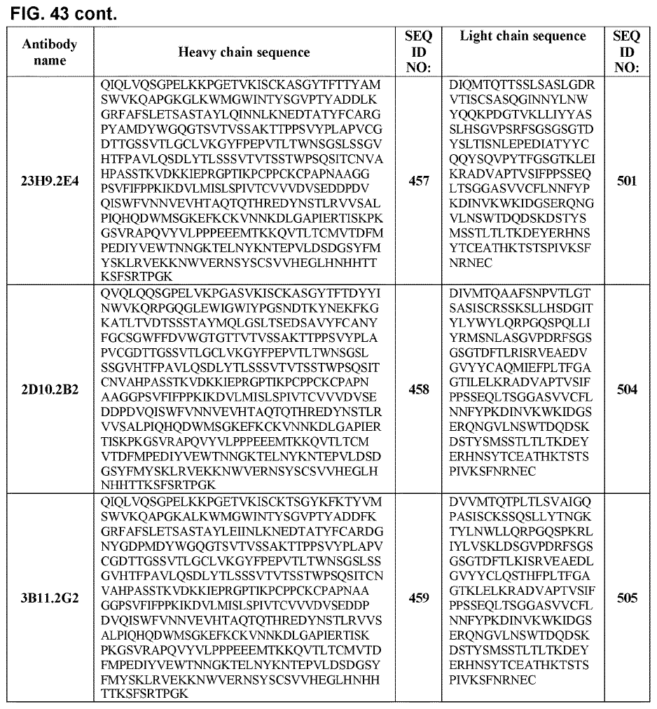

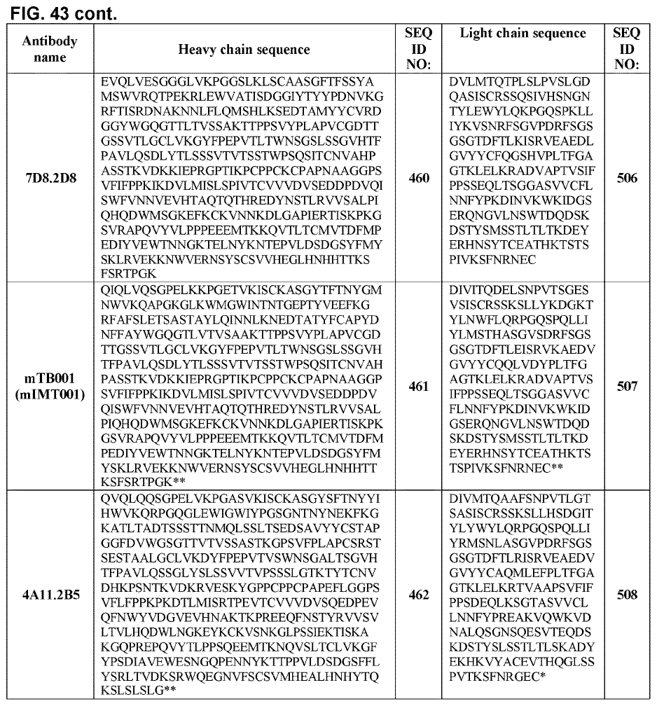

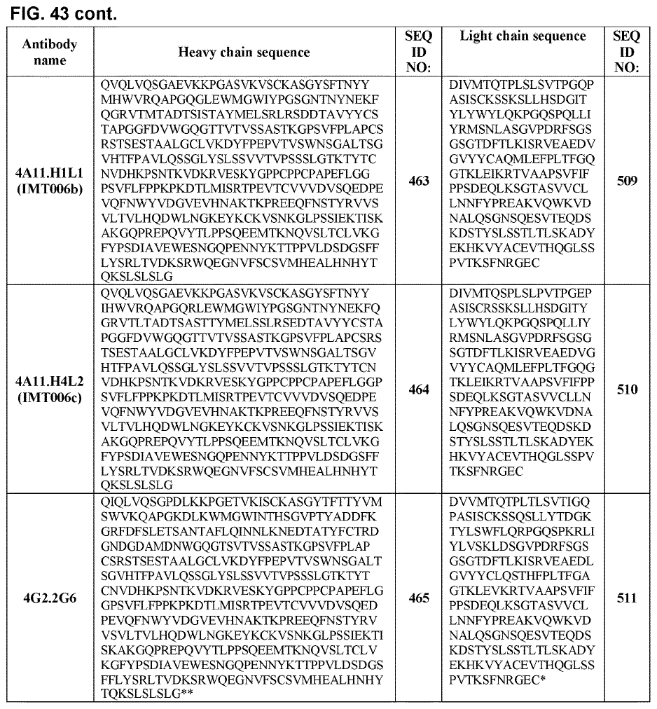

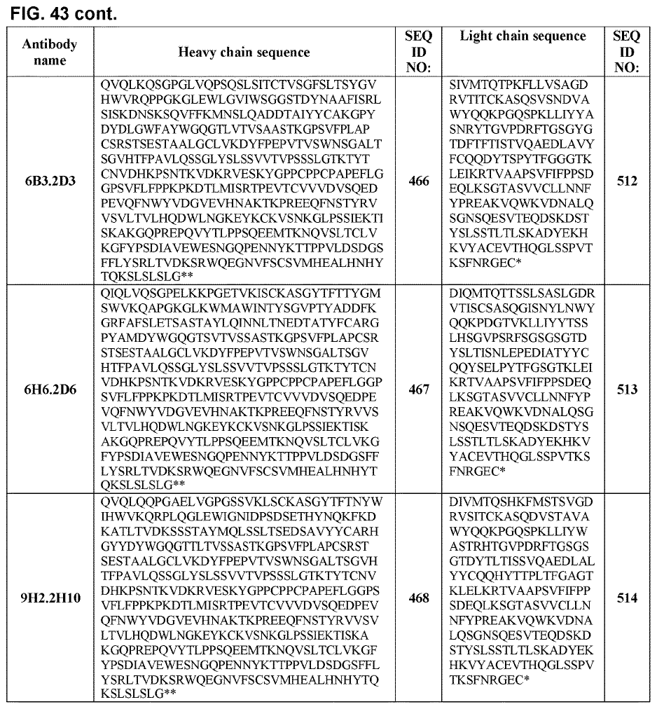

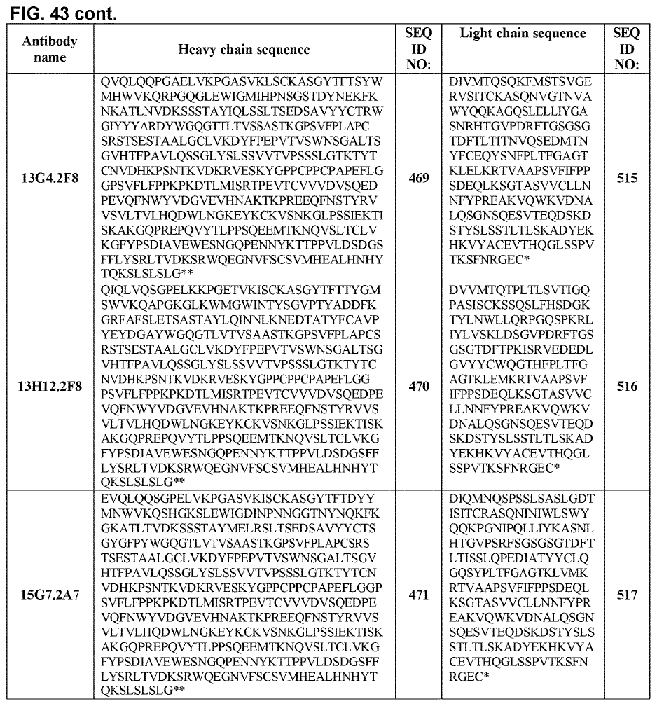

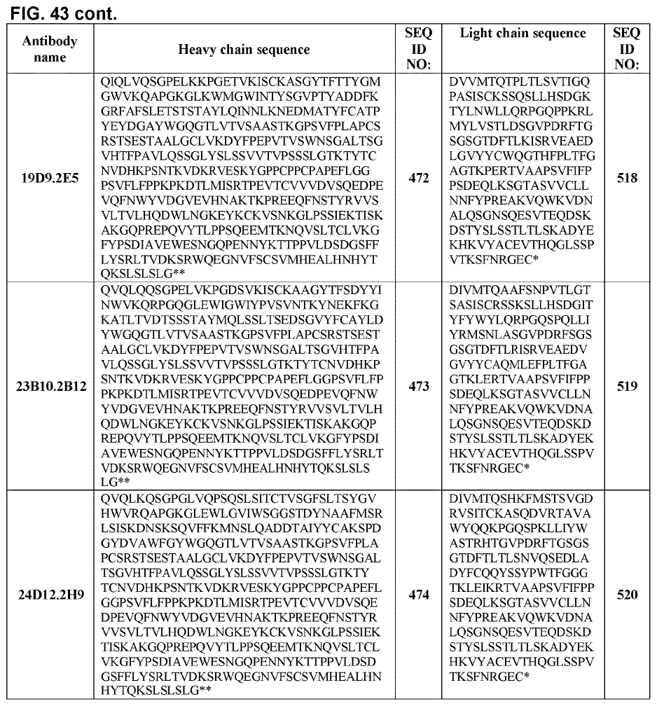

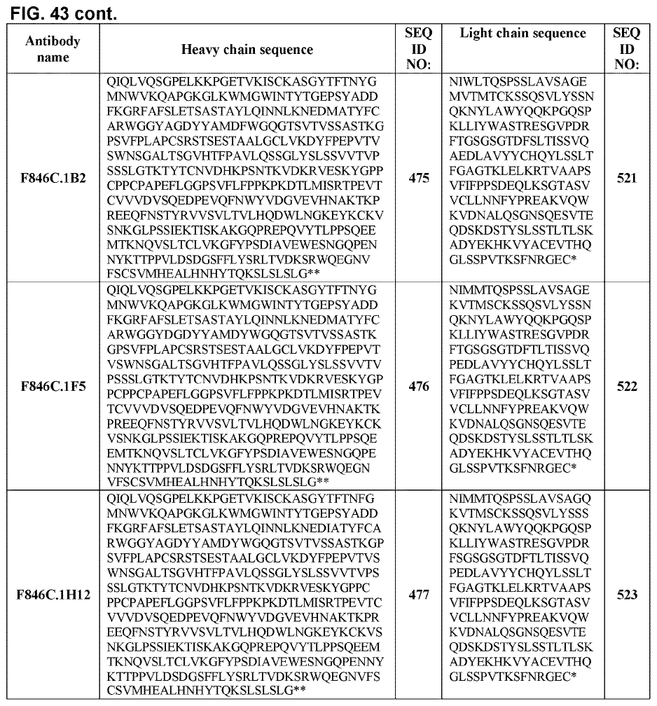

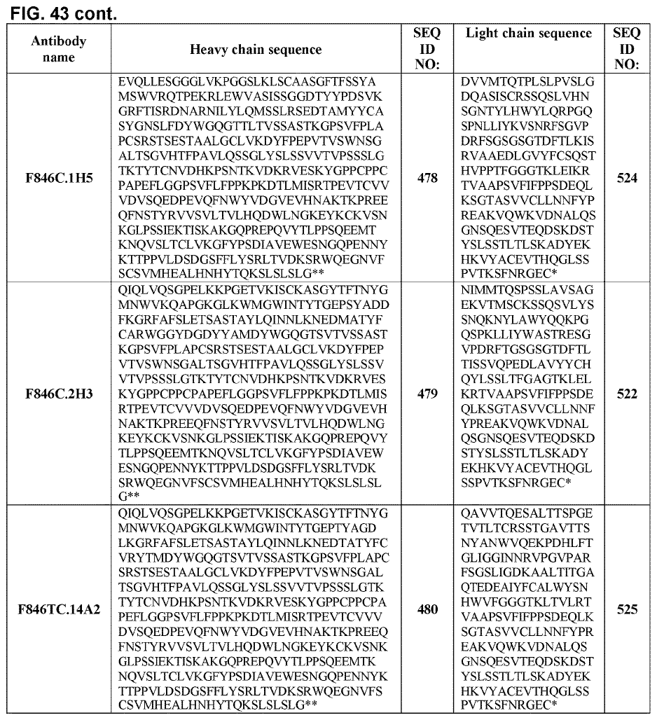

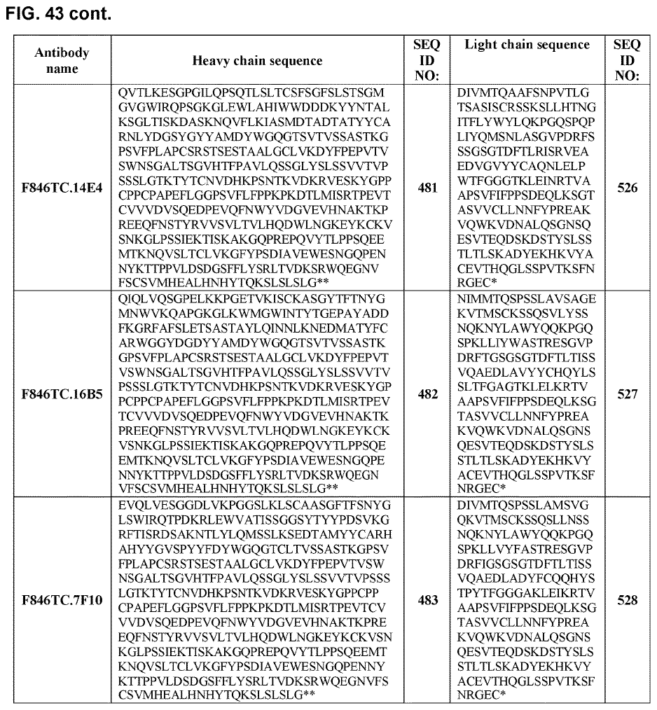

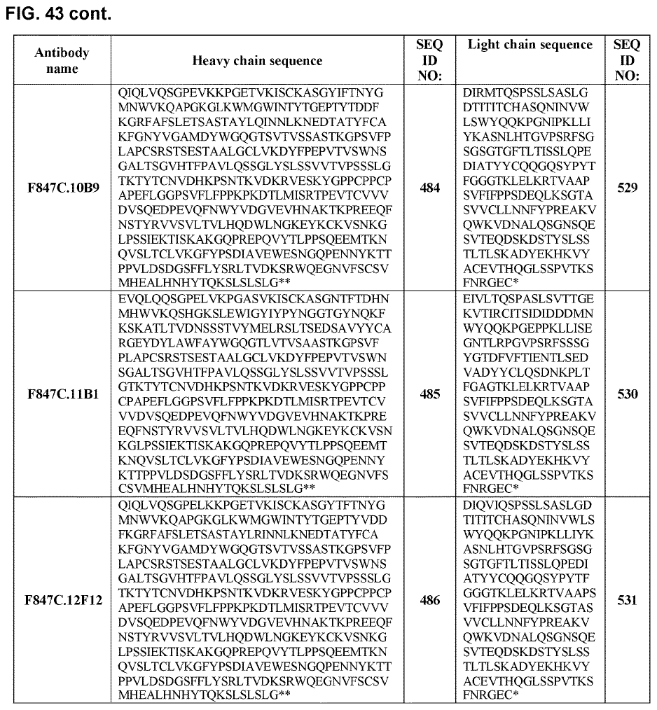

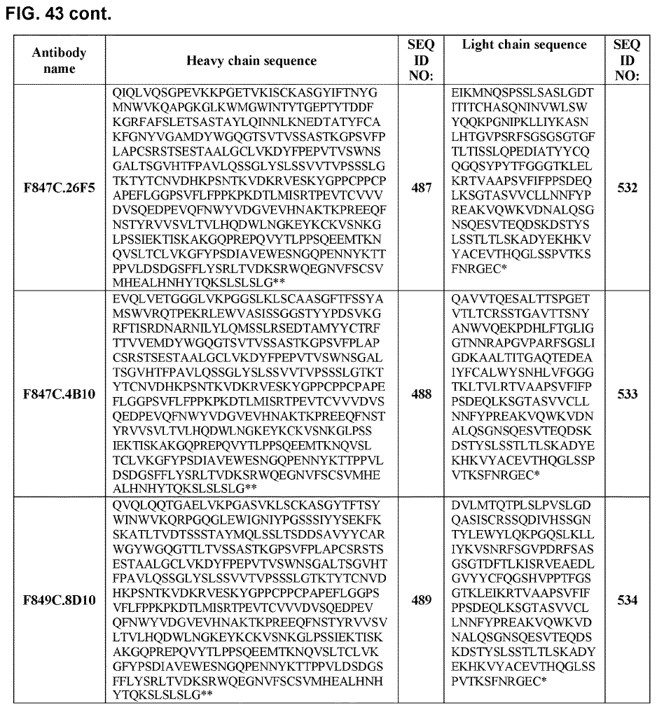

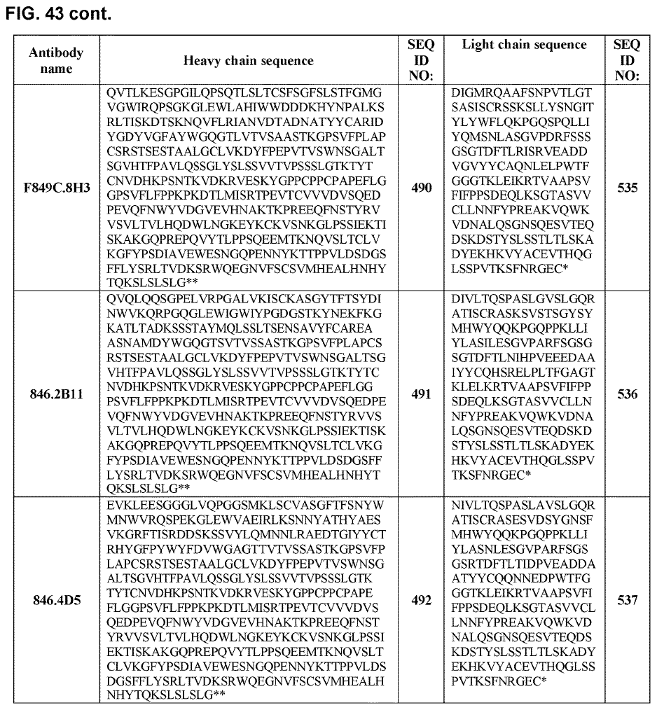

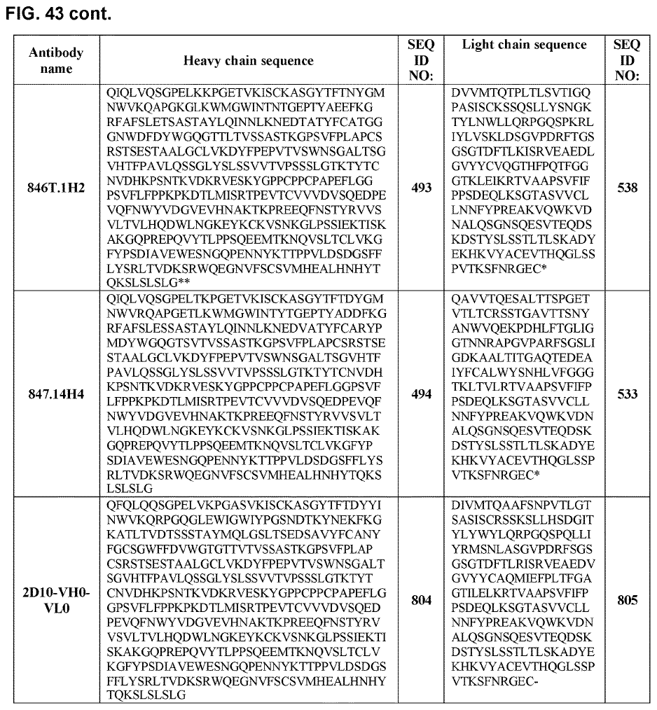

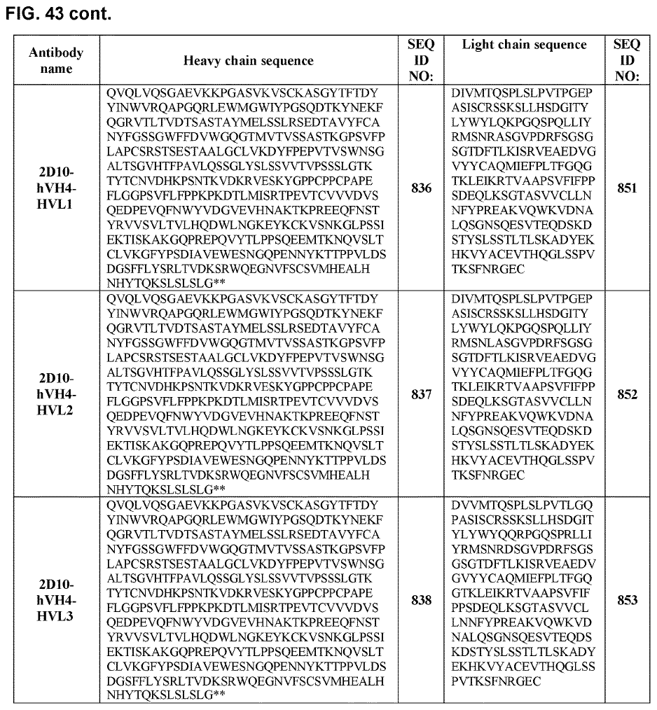

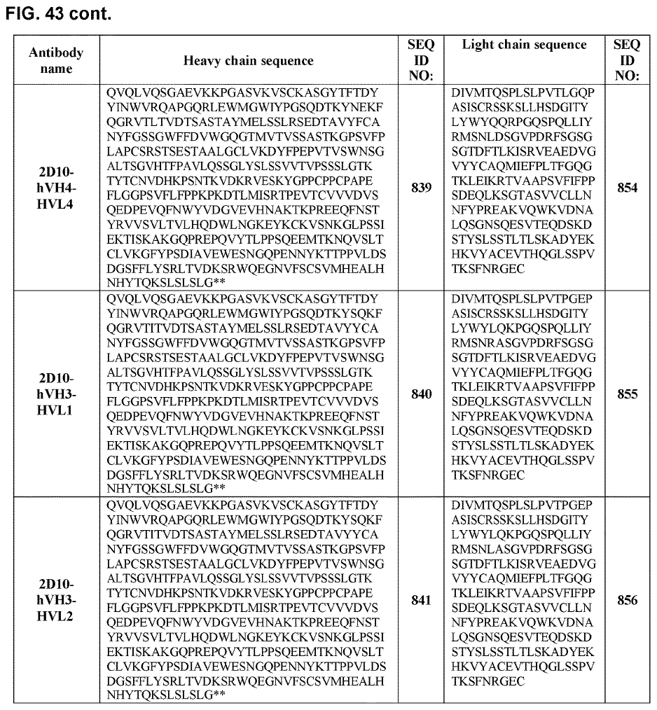

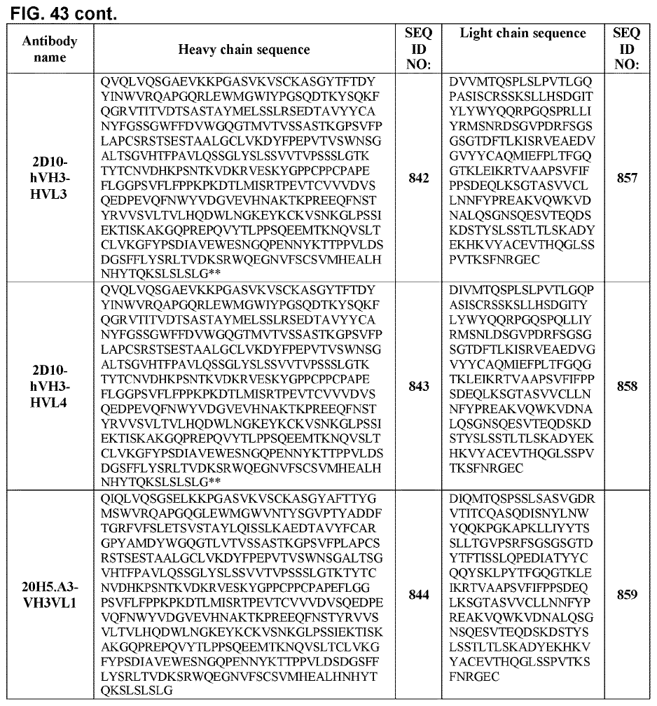

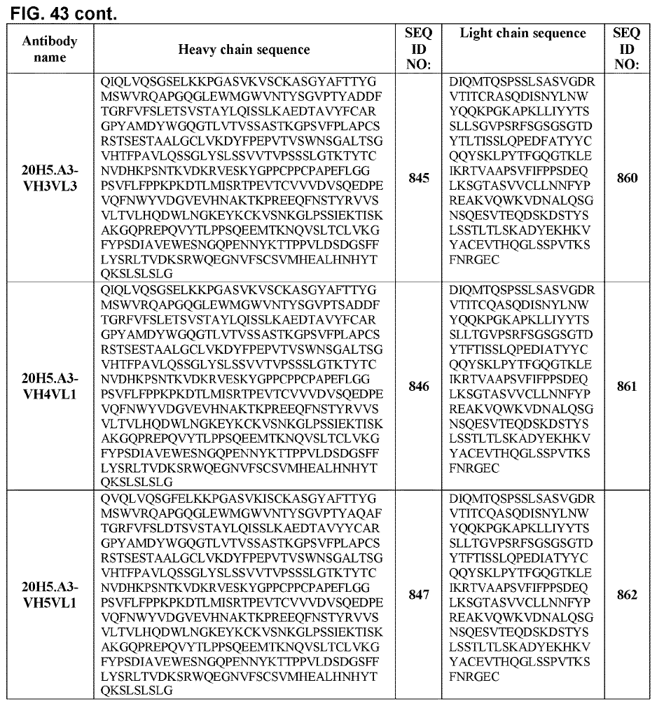

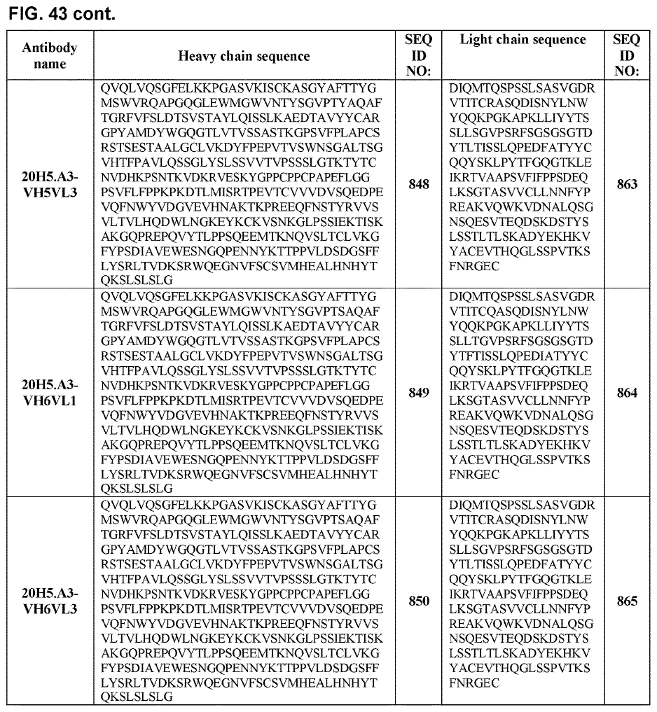

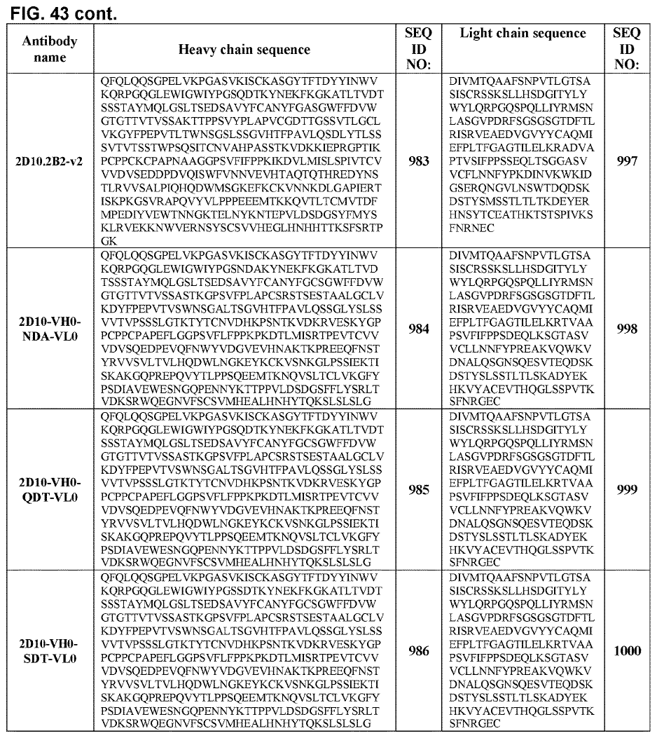

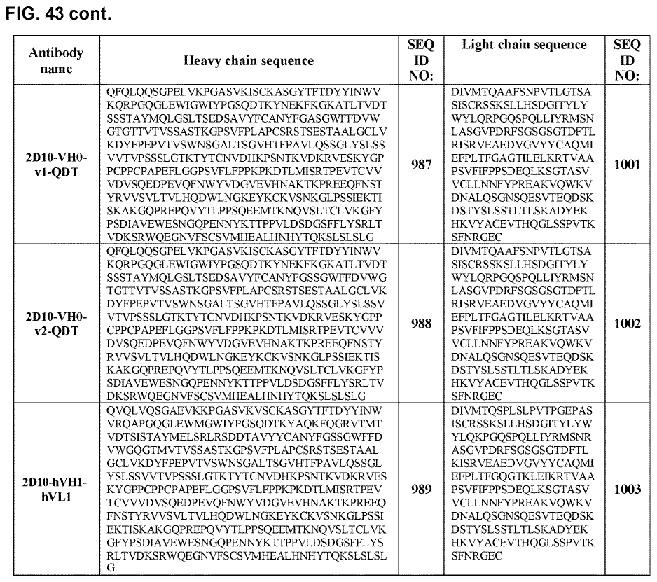

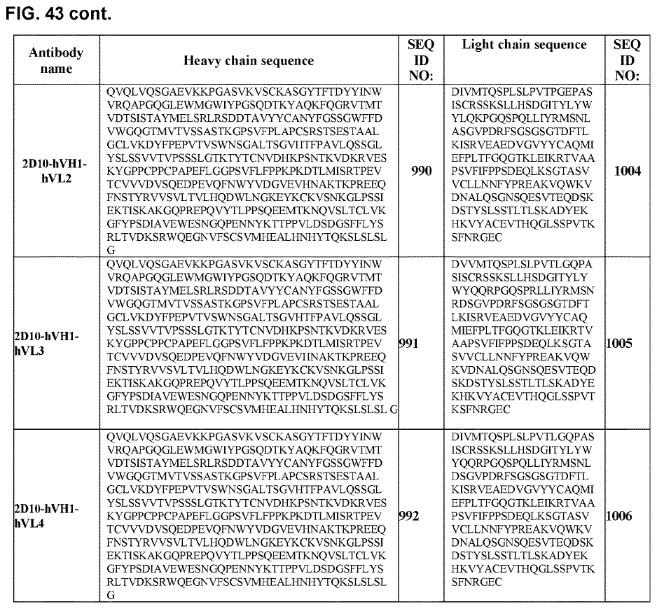

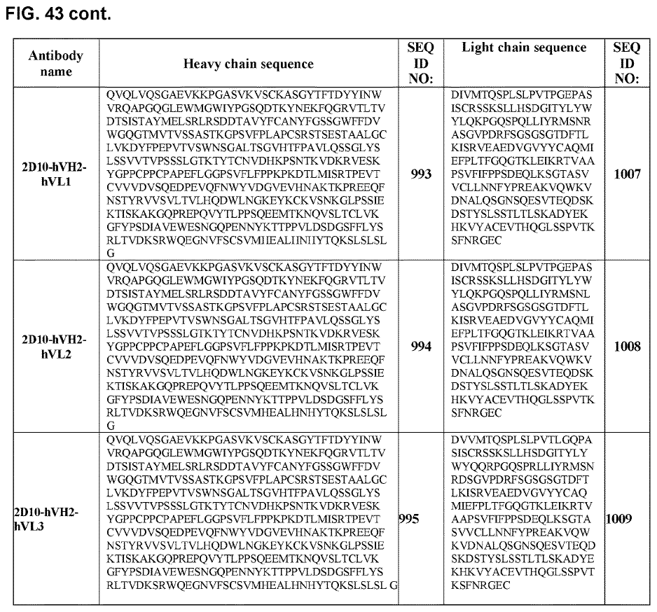

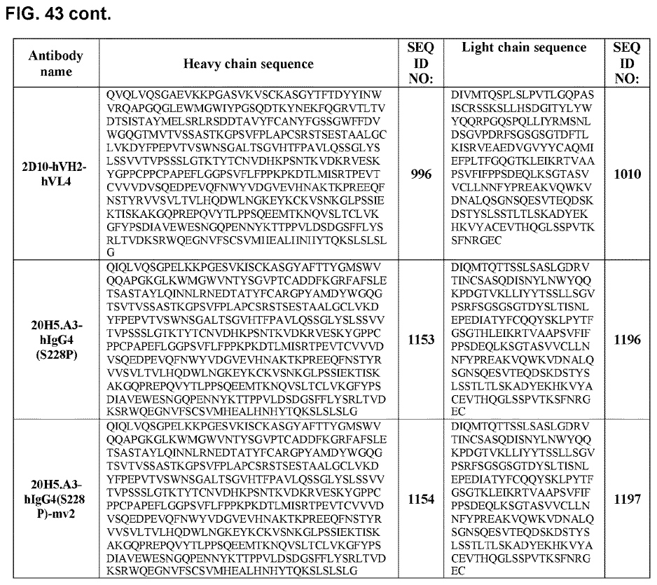

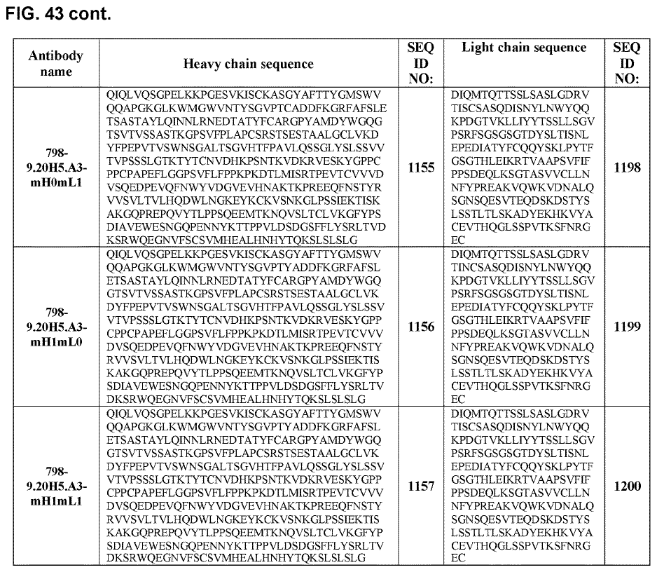

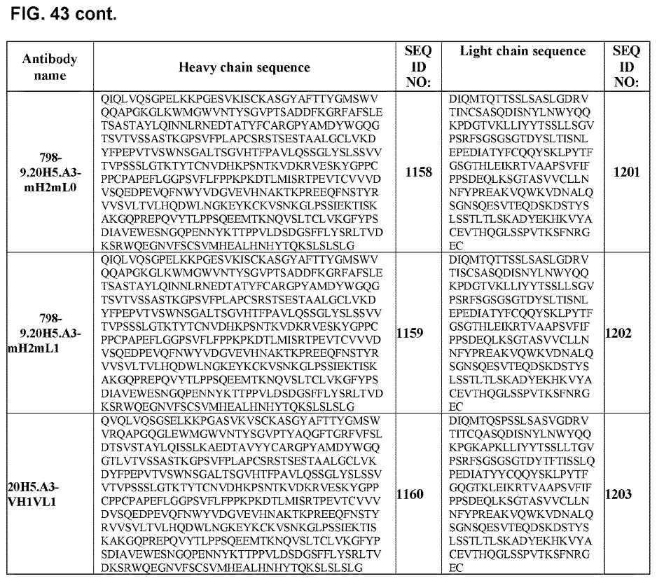

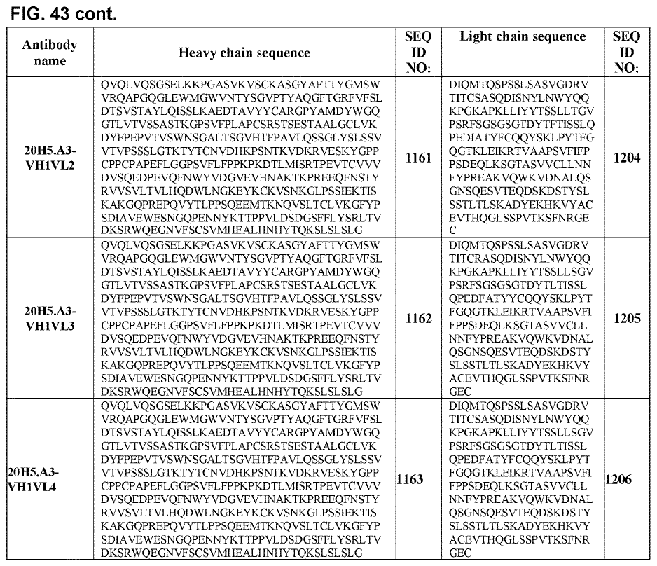

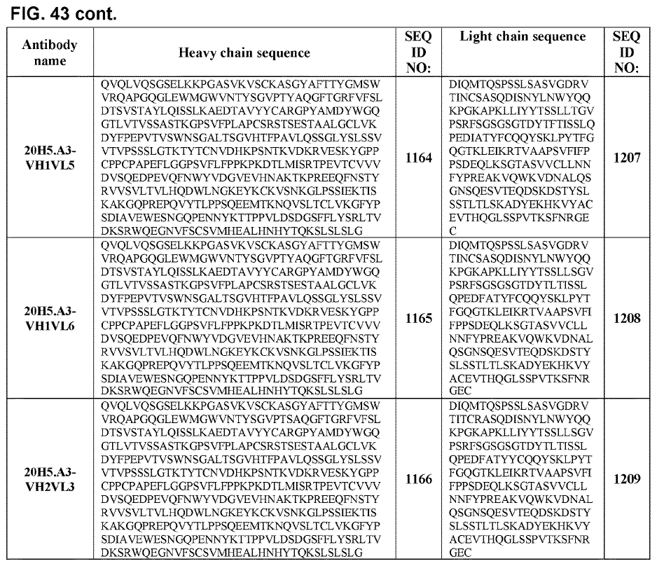

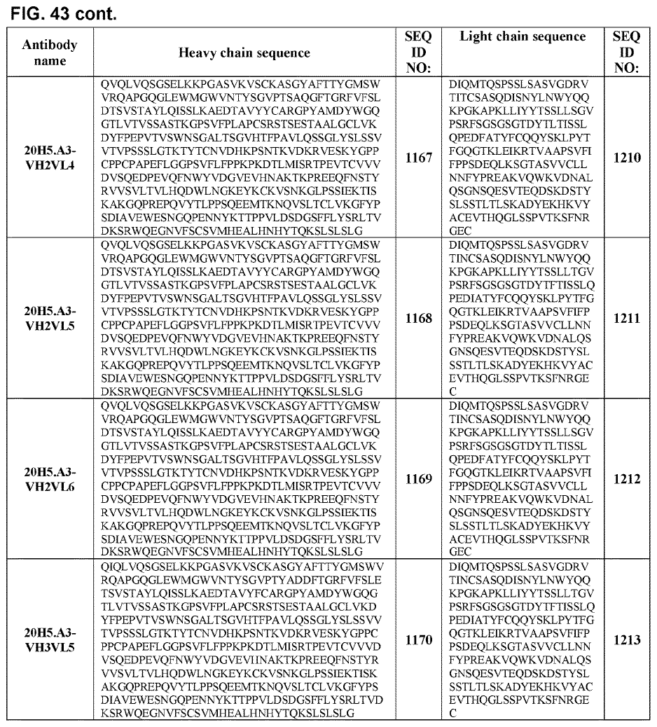

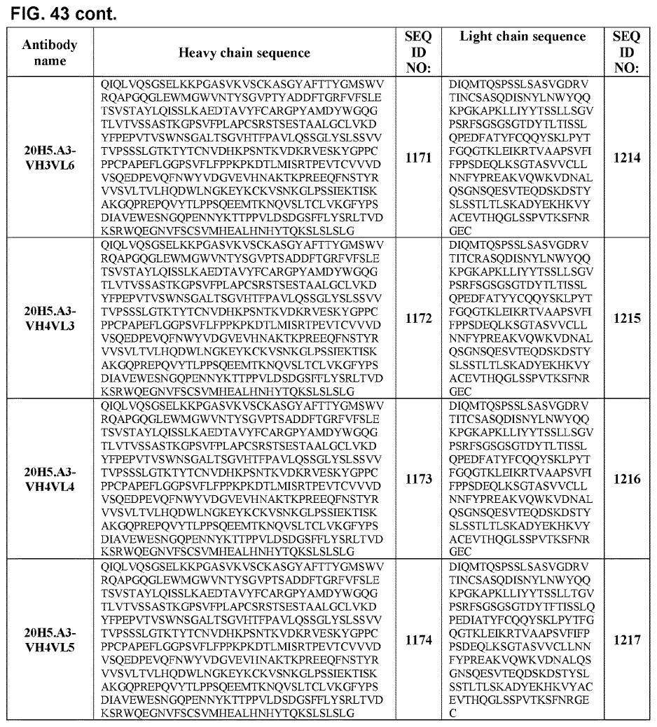

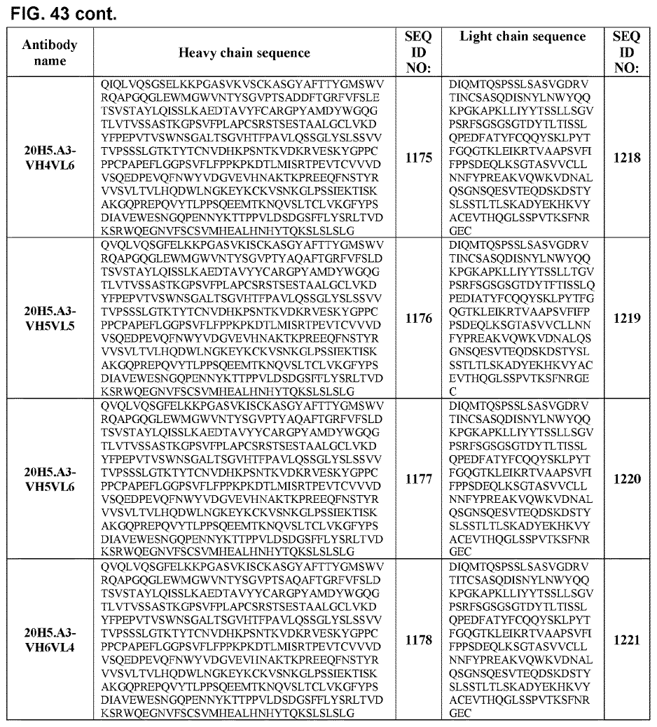

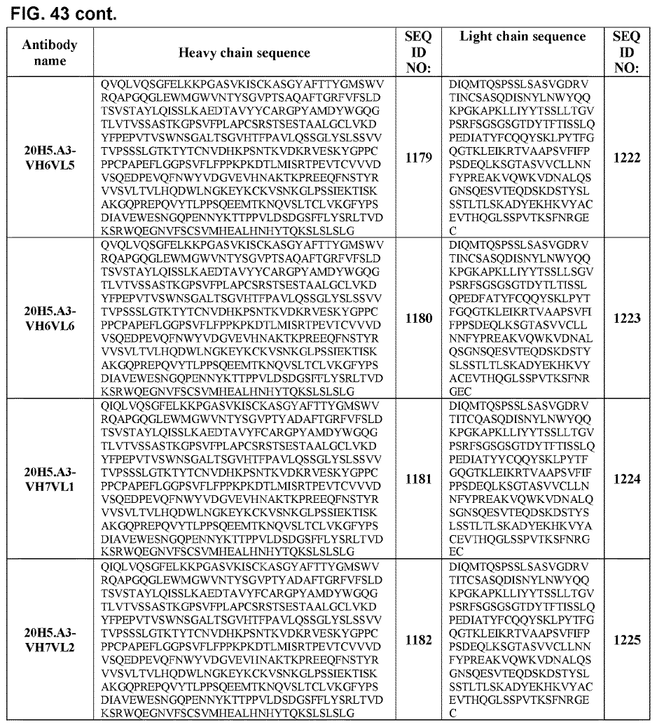

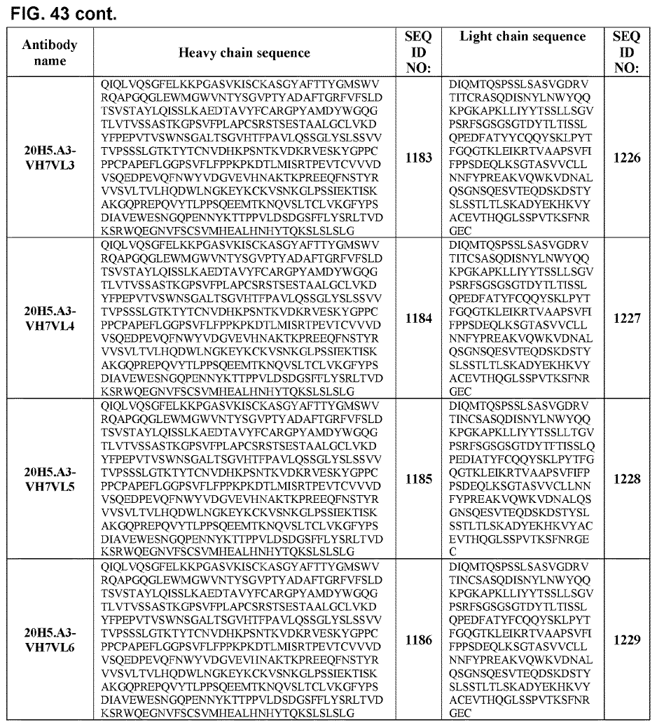

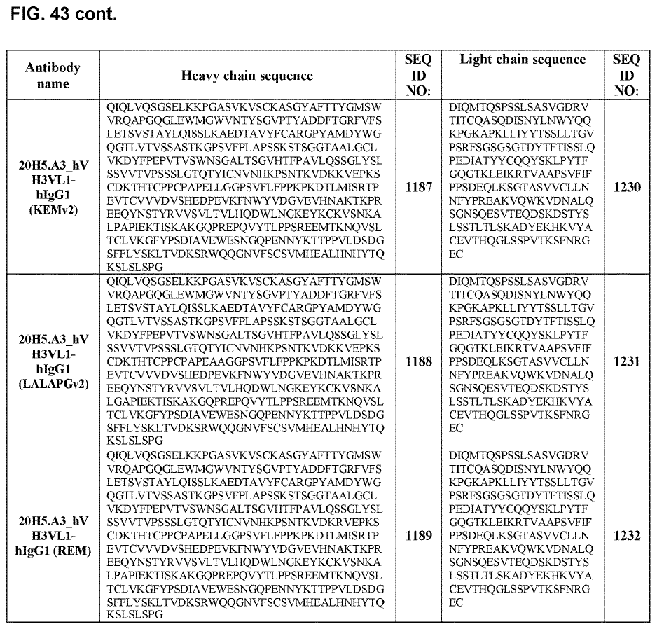

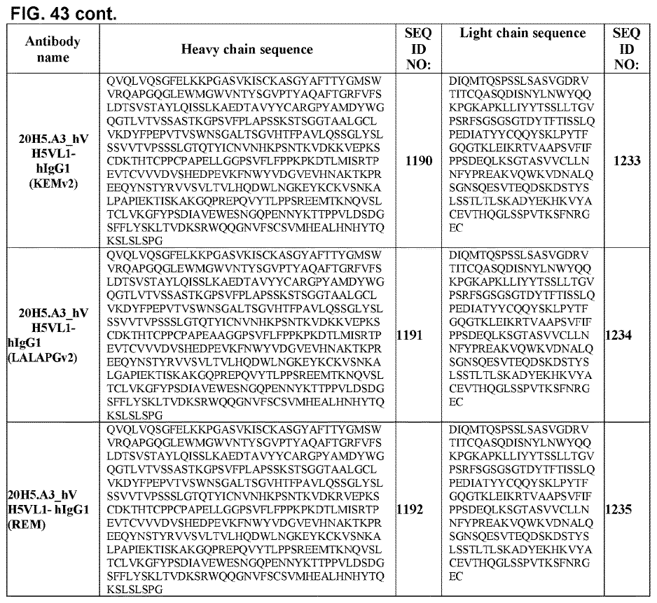

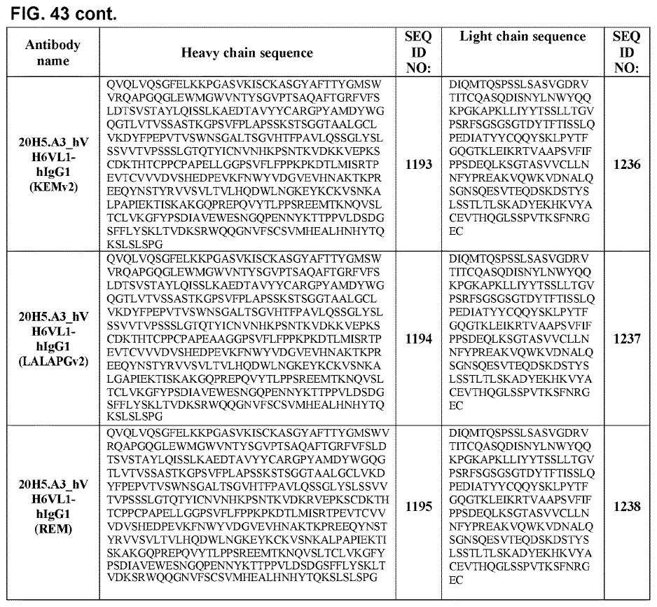

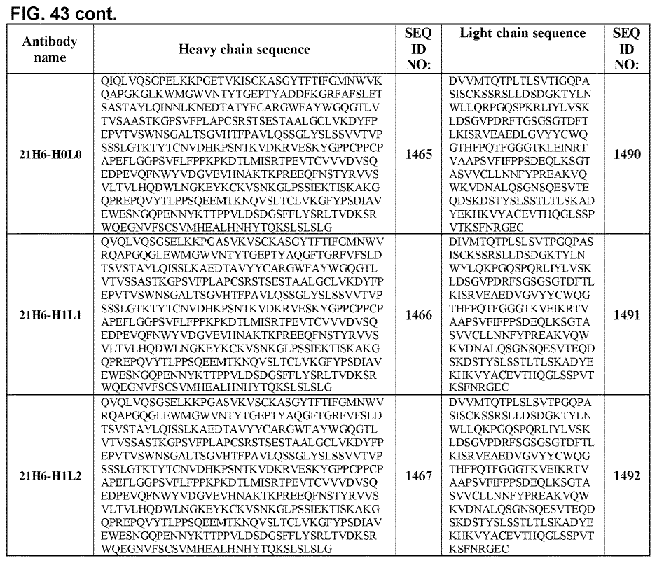

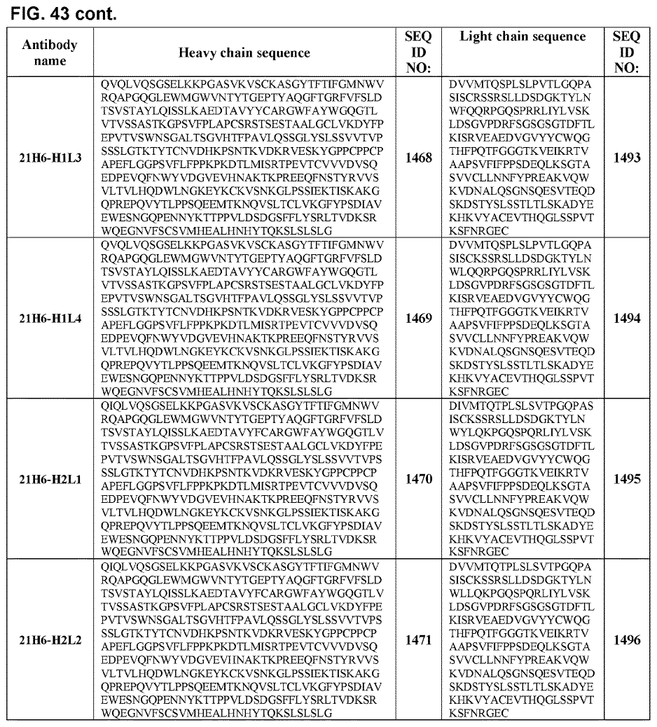

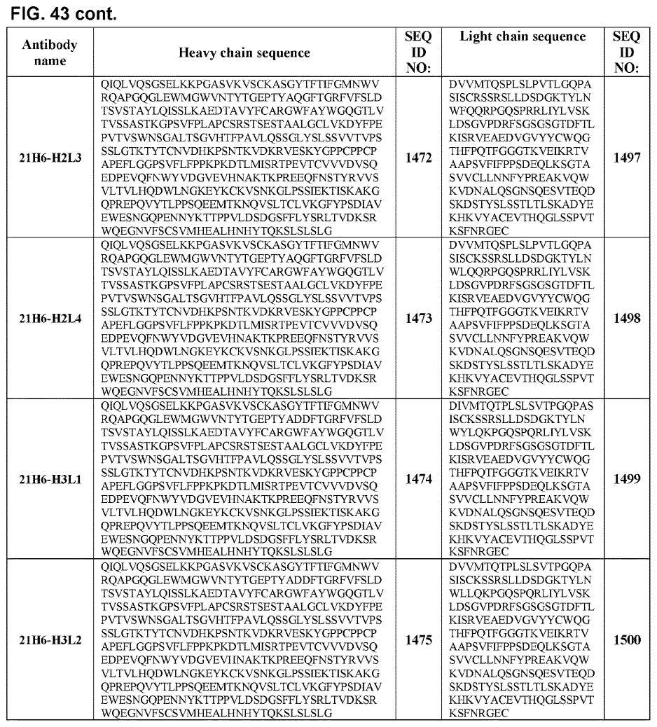

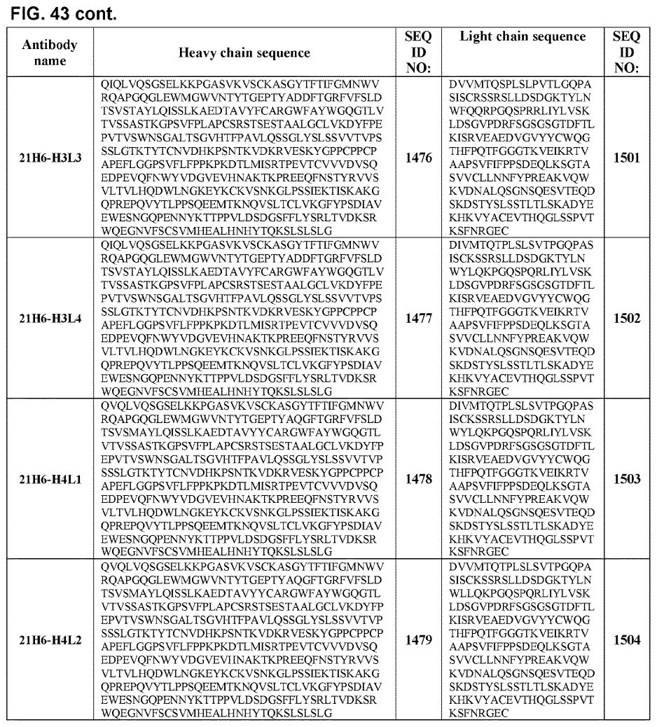

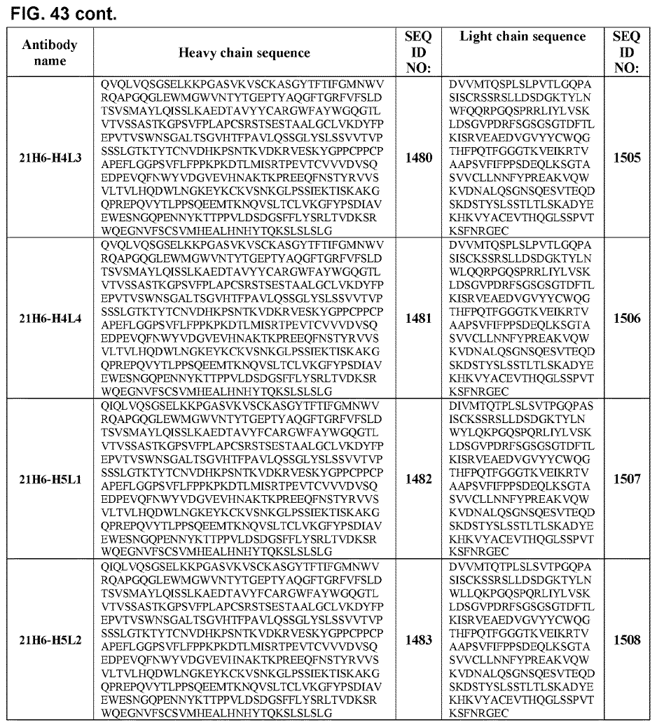

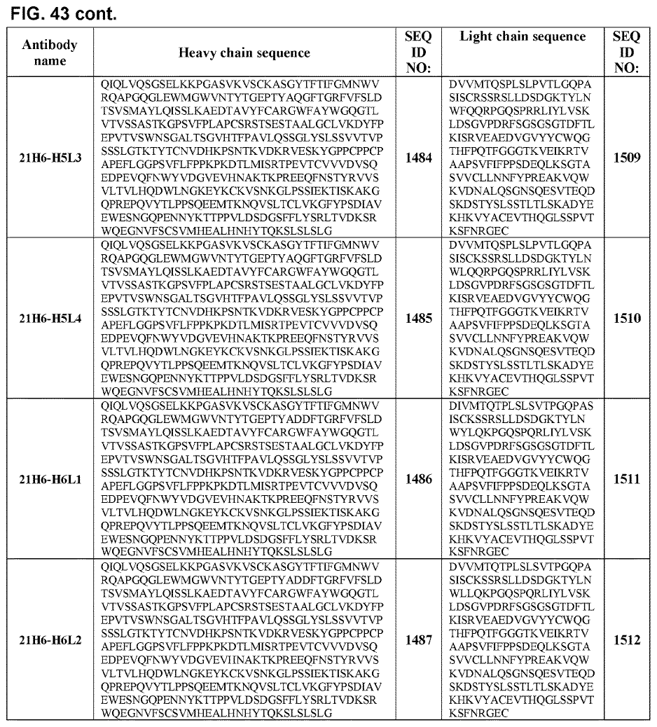

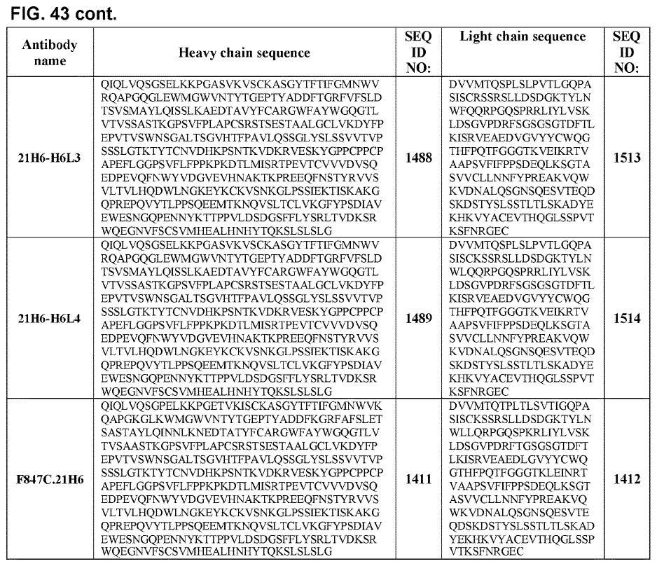

In some embodiments, the anti-Gal3 antibodies or binding fragments thereof comprise a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 27-70. In some embodiments, the V_{H}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 71-111, 801, 951, 952. In some embodiments, the V_{H}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 112-169, 802, 953, 954. In some embodiments, the V_{L}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 170-220. In some embodiments, the V_{L}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 211-247. In some embodiments, the V_{L}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 248-296. In some embodiments, the antibodies comprise one or more sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a VL sequence, a VH sequence, a VL/VH pairing, and/or V_{H}-CDR1, V_{H}-CDR2, V_{H}-CDR3, V_{L}-CDR1, V_{L}-CDR2, V_{L}-CDR3 (including 1, 2, 3, 4, or 5 amino acid substitutions of any one or more of these CDRs) set from the heavy chain and light chain sequences as depicted in FIG. 43.

In some embodiments, antibodies or binding fragments thereof are provided. In some embodiments, the antibodies or binding fragments thereof are anti-Gal3 antibodies or binding fragments thereof. In some embodiments, the anti-Gal3 antibodies or binding fragments thereof comprises a heavy chain variable region comprising a VH-CDR1, a VH-CDR2, and a VH-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the VH-CDR1 comprises an amino acid sequence having at least 0, 1, 2, 3, 4, 5, or 6 substitutions relative to any amino acid sequence according to SEQ ID NOs: 27-70. In some embodiments, the VH-CDR2 comprises an amino acid sequence having at least 0, 1, 2, 3, 4, 5, or 6 substitutions relative to any amino acid sequence according to SEQ ID NOs: 71-111, 801, 951, 952. In some embodiments, the VH-CDR3 comprises an amino acid sequence having at least 0, 1, 2, 3, 4, 5, or 6 substitutions relative to any amino acid sequence according to SEQ ID NOs: 112-169, 802, 953, 954. In some embodiments, the V_{L}-CDR1 comprises an amino acid sequence having at least 0, 1, 2, 3, 4, 5, or 6 substitutions relative to any amino acid sequence according to SEQ ID NOs: 170-220. In some embodiments, the V_{L}-CDR2 comprises an amino acid sequence having at least 0, 1, 2, 3, 4, 5, or 6 substitutions relative to any amino acid sequence according to SEQ ID NOs: 211-247. In some embodiments, the V_{L}-CDR3 comprises an amino acid sequence having at least 0, 1, 2, 3, 4, 5, or 6 substitutions relative to any amino acid sequence according to SEQ ID NOs: 248-296.

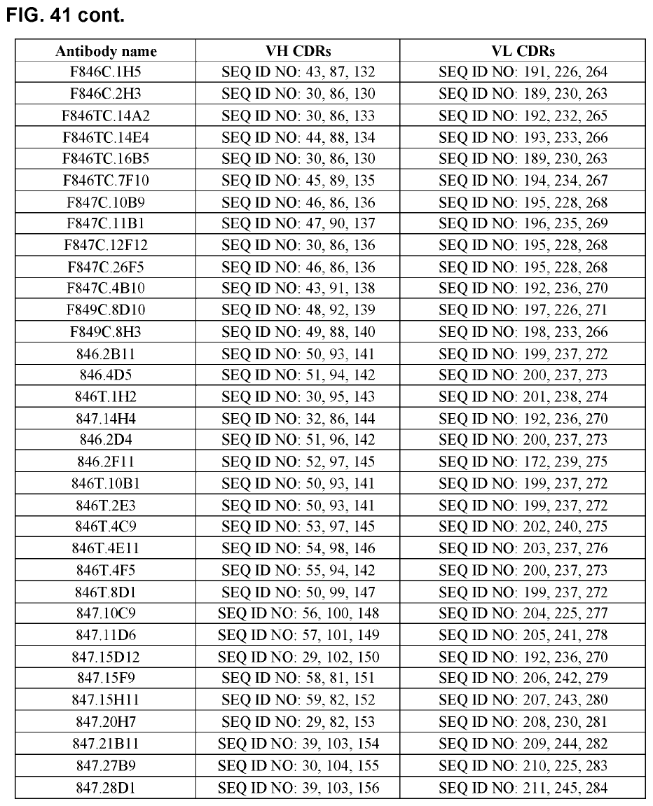

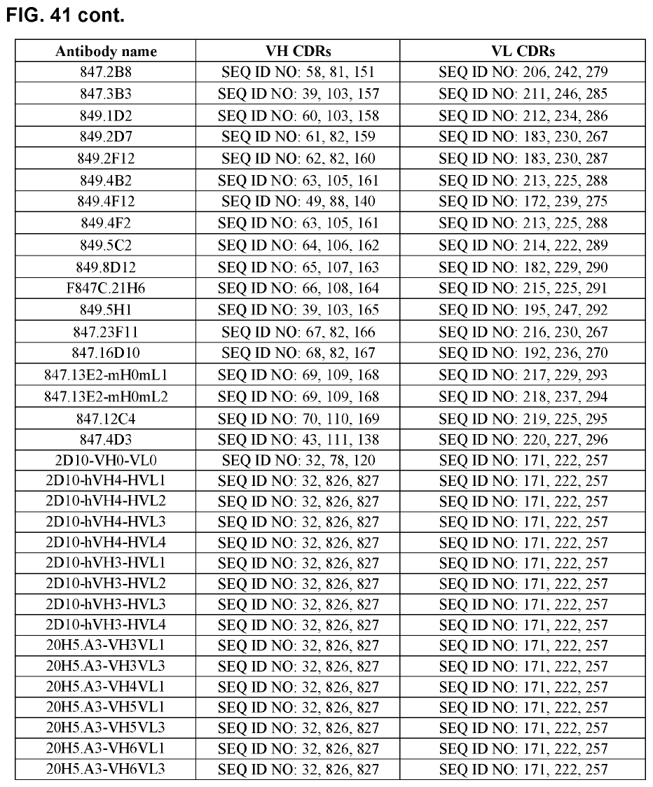

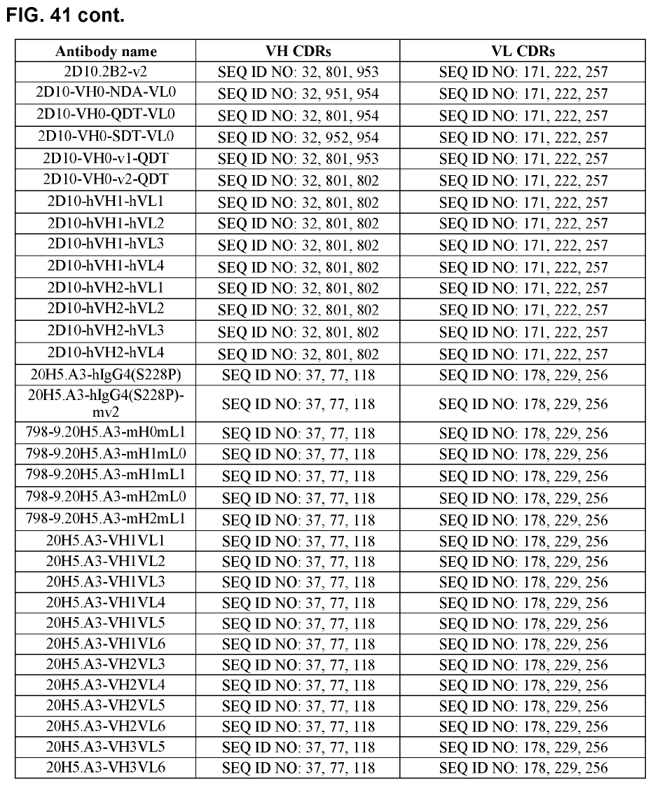

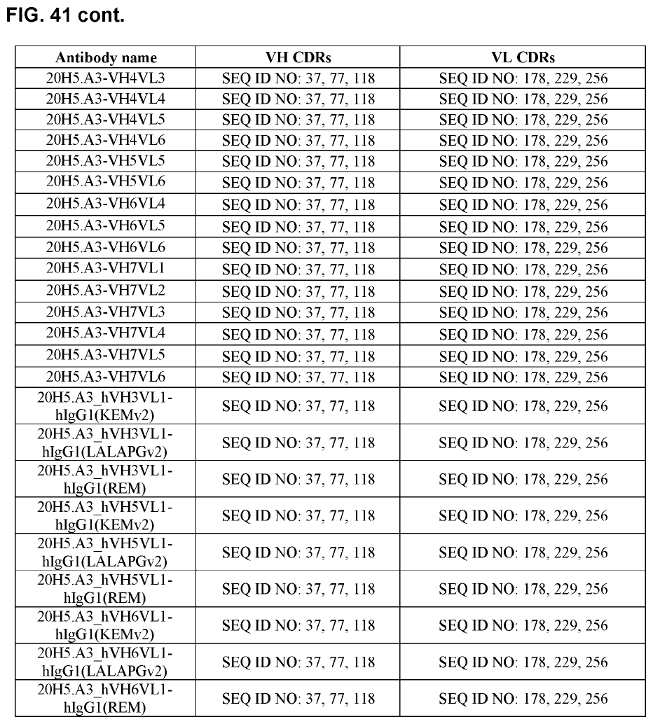

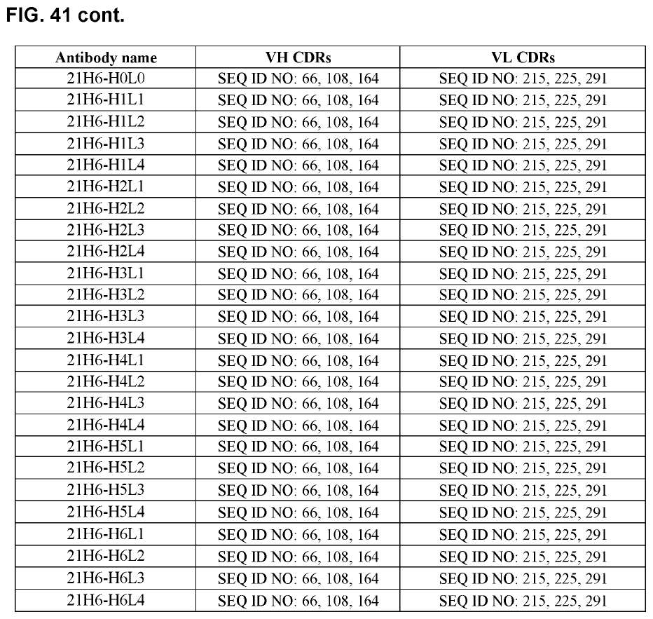

In some embodiments, the antibody or binding fragment thereof comprises a combination of a V_{L}-CDR1, a V_{L}-CDR2, a V_{L}-CDR3, a VH-CDR1, a VH-CDR2, and a VH-CDR3 as illustrated in FIG. 41.

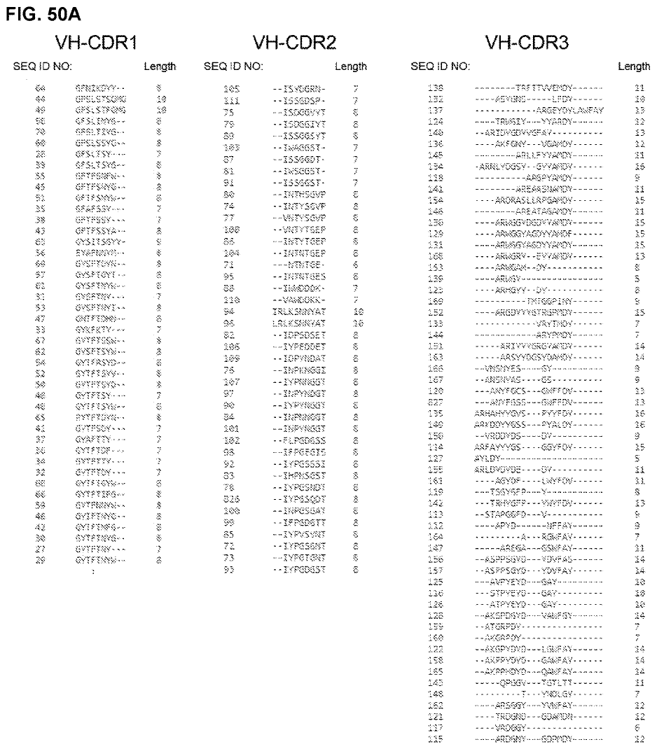

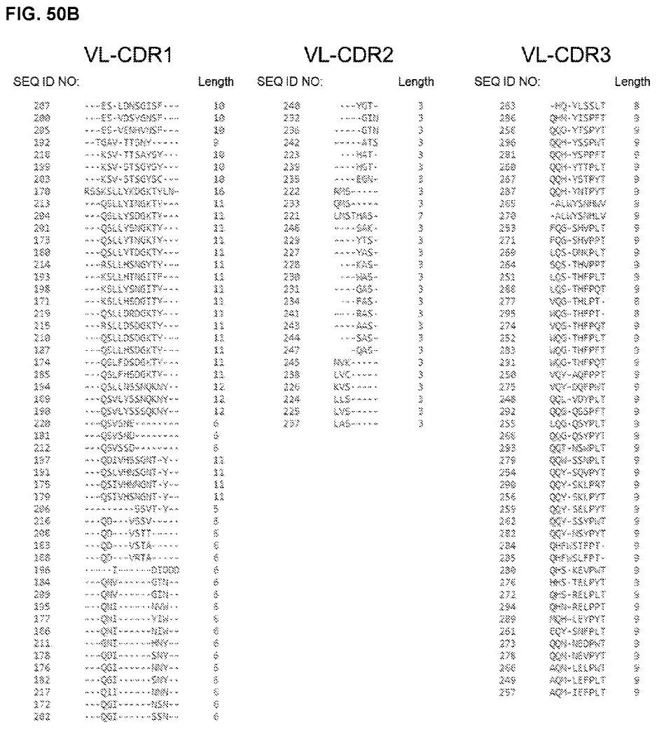

In some embodiments, the antibody or binding fragment thereof comprises a combination of a VH-CDR1, a VH-CDR2, a VH-CDR3, V_{L}-CDR1, a V_{L}-CDR2, and a V_{L}-CDR3, where one or more of these CDRs is defined by a consensus sequence. The consensus sequences provided herein have been derived from the alignments of CDRs depicted in FIG. 50A-B. However, it is envisioned that alternative alignments may be done (e.g. using global or local alignment, or with different algorithms, such as Hidden Markov Models, seeded guide trees, Needleman-Wunsch algorithm, or Smith-Waterman algorithm) and as such, alternative consensus sequences can be derived.

In some embodiments, the V_{H}-CDR1 is defined by the formula X₁X₂X₃X₄X₅X₆X₇X₈X₉X₁₀ (SEQ ID NO. 1915), where X₁ is E, G, or R; X₂ is F, N, or Y; X₃ is A, I, K, N, S, or T; X₄ is F, I, or L; X₅ is I, K, N, R, S, or T; X₆ is D, G, I, N, S, or T; X₇ is F, G, H, S, or Y; X₈ is no amino acid, A, D, G, I, M, N, T, V, W, or Y; X₉ is no amino acid, M, or Y; X₁₀ is no amino acid or G; In some embodiments, the VH-CDR1 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the VH-CDR1 comprises a sequence having 0, 1, 2, 3, 4, 5, or 6 substitutions from this consensus sequence.

In some embodiments, the VH-CDR2 is defined by the formula X₁X₂X₃X₄X₅X₆X₇X₈X₉X₁₀ (SEQ ID NO. 1916), where X₁ is no amino acid, I, or L; X₂ is no amino acid or R; X₃ is no amino acid, F, I, L, or V; X₄ is A, D, F, H, K, L, N, S, W, or Y; X₅ is A, D, P, S, T, W, or Y; X₆ is D, E, G, H, K, N, S, V, or Y; X₇ is D, E, G, N, S, or T; X₈ is D, G, I, K, N, Q, R, S, V, or Y; X₉ is A, D, E, G, I, K, N, P, S, T, V, or Y; X₁₀ is no amino acid, I, P, S, or T. In some embodiments, the V_{H}-CDR2 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the VH-CDR2 comprises a sequence having 0, 1, 2, 3, 4, 5, or 6 substitutions from this consensus sequence.

In some embodiments, the V_{H}-CDR3 is defined by the formula X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅ (SEQ ID NO. 1917), where X₁ is no amino acid or A; X₂ is no amino acid, A, R, or Y; X₃ is no amino acid, A, F, H, K, L, R, S, or V; X₄ is no amino acid, A, D, K, N, R, S, or T; X₅ is no amino acid, A, D, G, H, I, L, N, P, R, S, T, V, or Y; X₆ is no amino acid, A, D, G, H, K, N, P, Q, R, S, or Y; X₇ is no amino acid, D, F, G, H, P, R, S, W, or Y; X₈ is no amino acid, A, D, E, G, I, R, or S; X₉ is no amino acid, A, C, D, E, F, G, I, N, R, S, T, V, or Y; X₁₀ is no amino acid, A, D, M, P, R, S, T, V, or Y; X₁₁ is no amino acid, A, D, E, F, L, T, V, or Y; X₁₂ is no amino acid, A, G, L, M, R, or T; X₁₃ is no amino acid, A, D, E, F, G, R, S, T, or V; X₁₄ is no amino acid, A, D, G, L, P, Q, R, S, T, V, or Y; X₁₅ is no amino acid, A, D, G, N, S, V, W, or Y; X₁₆ is no amino acid, A, D, E, F, L, P, T, V, W, or Y; X₁₇ is no amino acid, F, I, L, M, R, or Y; X₁₈ is no amino acid, A, D, G, N, or T; X₁₉ is no amino acid, F, N, S, T, V, or Y; X₂₀ is no amino acid or L; X₂₁ is no amino acid or A; X₂₂ is no amino acid or W; X₂₃ is no amino acid or F; X₂₄ is no amino acid or A; X₂₅ is no amino acid or Y. In some embodiments, the V_{H}-CDR3 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{H}-CDR3 comprises a sequence having 0, 1, 2, 3, 4, 5, or 6 substitutions from this consensus sequence.

In some embodiments, the V_{L}-CDR1 is defined by the formula X₁X₂X₃X₄X₅X₆X₇X₈X₉X₁₀X₁₁X₁₂X₁₃X₁₄X₁₅X₁₆X₁₇ (SEQ ID NO. 1918), where X₁ is no amino acid or R; X₂ is no amino acid or S; X₃ is no amino acid, S, or T; X₄ is no amino acid, E, G, K, Q, or R; X₅ is no amino acid, A, D, G, I, N, or S; X₆ is no amino acid, I, L, or V; X₇ is no amino acid, F, L, S, or V; X₈ is no amino acid, D, E, H, N, S, T, or Y; X₉ is no amino acid, D, E, I, K, N, R, S, T, or V; X₁₀ is no amino acid, D, H, N, R, S, or Y; X₁₁ is no amino acid, A, G, N, S, T, or V; X₁₂ is no amino acid, A, I, K, N, Q. T, V, or Y; X₁₃ is no amino acid, D, G, H, K, N, S, T, or Y; X₁₄ is no amino acid, C, F, I, N, S, T, V, or Y; X₁₅ is no amino acid, D, L, N, W, or Y; X₁₆ is no amino acid, N, or D; X₁₇ is no amino acid or D. In some embodiments, the V_{L}-CDR1 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{L}-CDR1 comprises a sequence having 0, 1, 2, 3, 4, 5, or 6 substitutions from this consensus sequence.

In some embodiments, the V_{L}-CDR2 is defined by the formula X₁X₂X₃X₄X₅X₆X₇X₈ (SEQ ID NO. 1919), where X₁ is no amino acid, K, L, N, Q, or R; X₂ is no amino acid, A, L, M, or V; X₃ is no amino acid, C, K, or S; X₄ is no amino acid or T; X₅ is no amino acid, A, E, F, G, H, K, Q, R, S, W, or Y; X₆ is no amino acid, A, G, or T; X₇ is no amino acid, I, K, N, S, or T; X₈ is no amino acid, N, or S. In some embodiments, the V_{L}-CDR2 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{L}-CDR2 comprises a sequence having 0, 1, 2, 3, 4, 5, or 6 substitutions from this consensus sequence.

In some embodiments, the V_{L}-CDR3 is defined by the formula X₁X₂X₃X₄X₅X₆X₇X₈X₉X₁₀ (SEQ ID NO. 1920), where X₁ is no amino acid, A, E, F, H, L, M, Q, S, V, or W; X₂ is A, H, or Q; X₃ is D, F, G, H, L, M, N, Q, S, T, W, or Y; X₄ is no amino acid or W; X₅ is A, D, I, K, L, N, Q, R, S, T, V, or Y; X₆ is D, E, H, I, K, L, N, Q, S, or T; X₇ is D, F, K, L, N, P. S, T, V, W, or Y; X₈ is H, P, or S; X₉ is F, L, P, Q, R, T, W, or Y; X₁₀ is no amino acid, T, or V. In some embodiments, the V_{L}-CDR3 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the VL-CDR3 comprises a sequence having 0, 1, 2, 3, 4, 5, or 6 substitutions from this consensus sequence.

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence selected from SEQ ID NOs: 297-373, 803, 806-820, 955-968, 1067-1109, 1415-1439. In some embodiments, the light chain variable region of the antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence selected from SEQ ID NOs: 374-447, 821-835, 941-943, 969-982, 1110-1152, 1440-1464. In some embodiments, the antibodies or binding fragments thereof are anti-Gal3 antibodies or binding fragments thereof.

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to the sequence selected from SEQ ID NOs: 297-373, 803, 806-820, 955-968, 1067-1109, 1415-1439. In some embodiments, the light chain variable region of the antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to the sequence selected from SEQ ID NOs: 374-447, 821-835, 941-943, 969-982, 1110-1152, 1440-1464. In some embodiments, the antibodies or binding fragments thereof are anti-Gal3 antibodies or binding fragments thereof.

In some embodiments, the antibodies comprise one or more sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a VL sequence, a VH sequence, a VL/VH pairing, and/or V_{L}-CDR1, V_{L}-CDR2, V_{L}-CDR3, V_{H}-CDR1, V_{H}-CDR2, V_{H}-CDR3 (including 1, 2, 3, 4, or 5 amino acid substitutions of any one or more of these CDRs) set from the heavy chain and light chain sequences as depicted in FIG. 43.

In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises one of the amino acid sequences of SEQ ID NOs: 27-70, the V_{H}-CDR2 comprises one of the amino acid sequences of SEQ ID NOs: 71-111, 801, 951, 952, the V_{H}-CDR3 comprises one of the amino acid sequences of SEQ ID NO: 112-169, 802, 953, 954, the V_{L}-CDR1 comprises one of the amino acid sequences of SEQ ID NOs: 170-220, the V_{L}-CDR2 comprises one of the amino acid sequences of SEQ ID NOs: 211-247, the V_{L}-CDR3 comprises one of the amino acid sequences of SEQ ID NOs: 248-296, the heavy chain variable region has a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to one of the amino acid sequences of SEQ ID NOs: 374-447, 821-835, 969-982, 1110-1152, 1440-1464, and the light chain variable region has a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to one of the amino acid sequences of SEQ ID NOS: 374-447, 821-835, 927-929.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence selected from SEQ ID NOs: 448-494, 804, 836-850, 983-996, 1153-1195, 1465-1489. In some embodiments, the antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1490-1514. In some embodiments, the antibodies or binding fragments thereof are anti-Gal3 antibodies or binding fragments thereof.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to the sequence selected from SEQ ID NOs: 448-494, 804, 836-850, 983-996, 1153-1195, 1465-1489. In some embodiments, the antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% similarity to the sequence selected from SEQ ID NOs: 495-538, 805, 851-865, 997-1010, 1196-1238, 1490-1514. In some embodiments, the antibodies or binding fragments thereof are anti-Gal3 antibodies or binding fragments thereof.

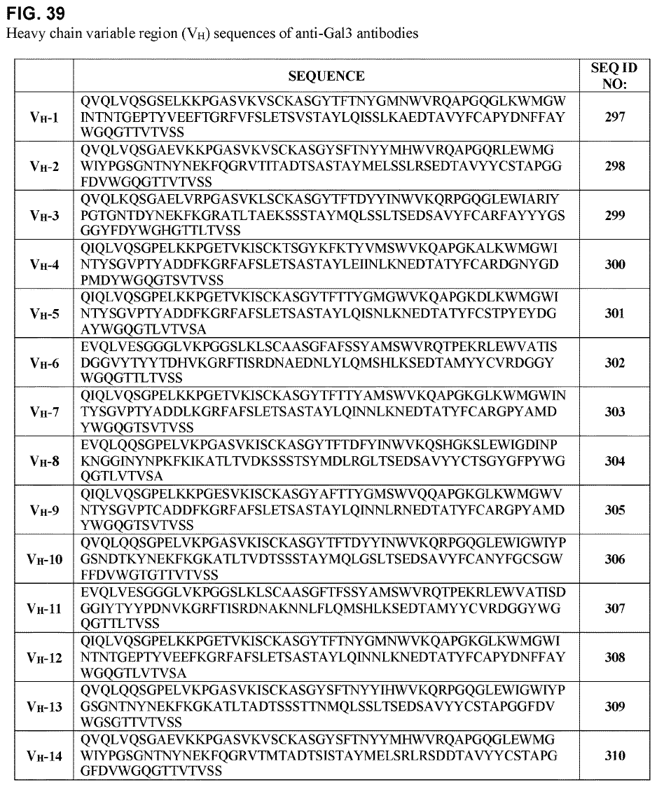

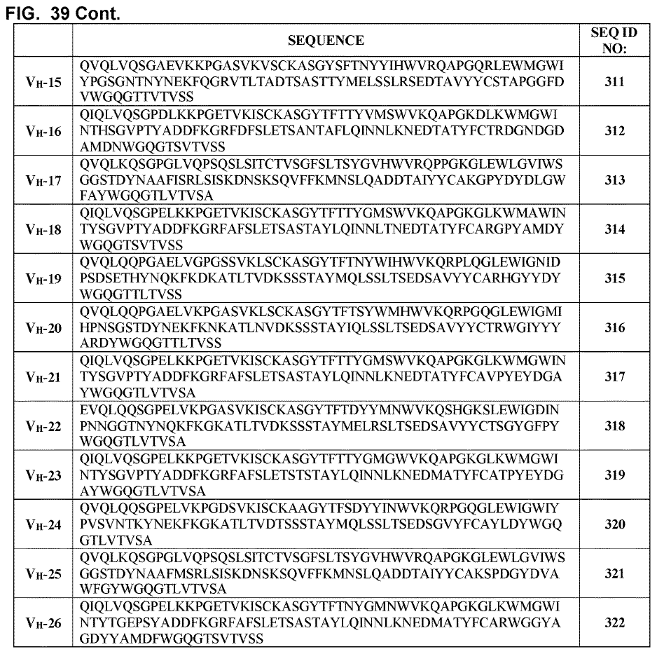

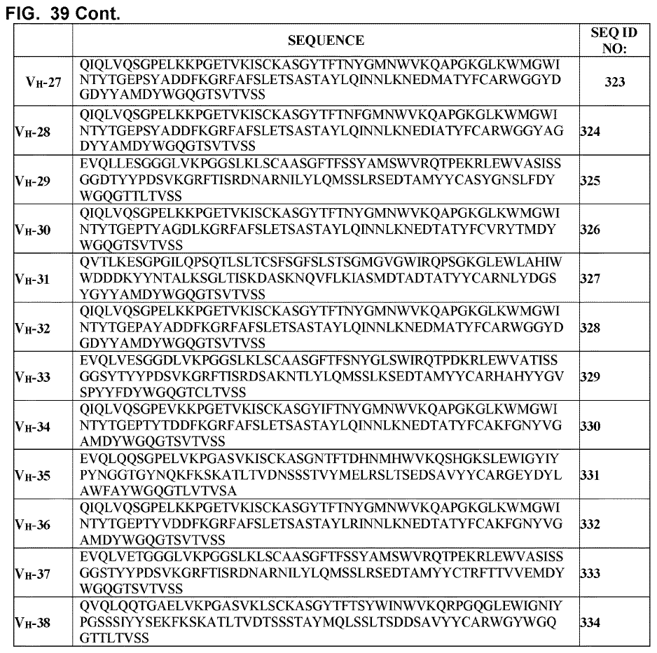

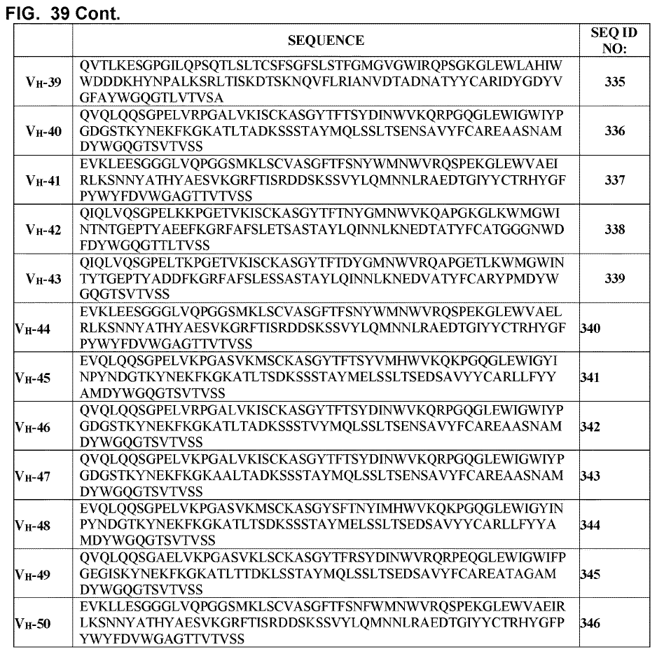

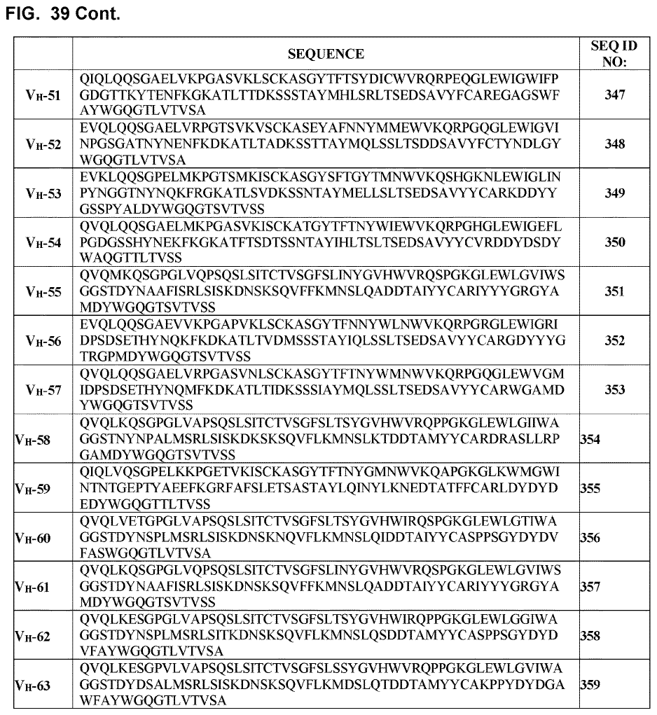

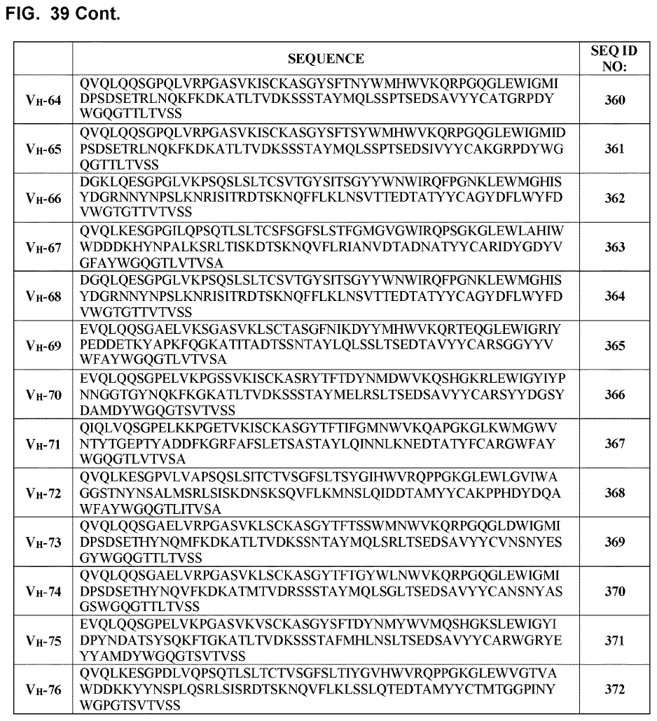

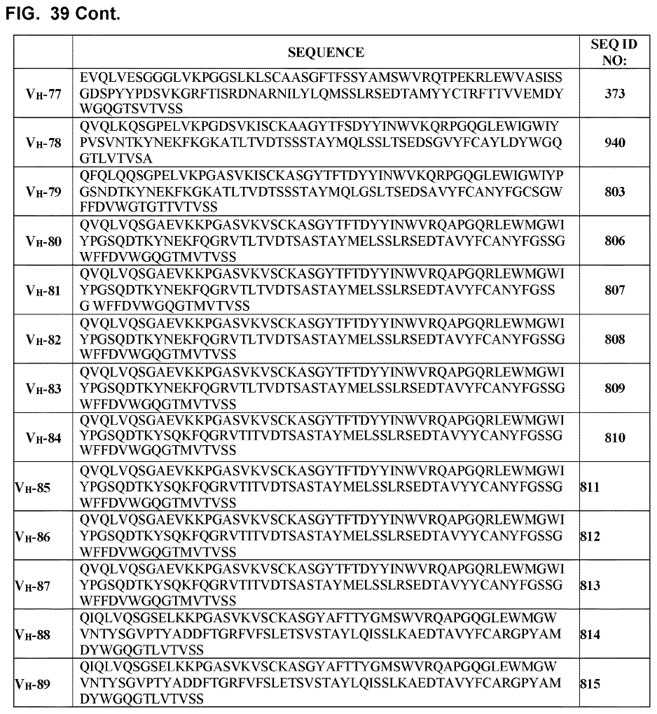

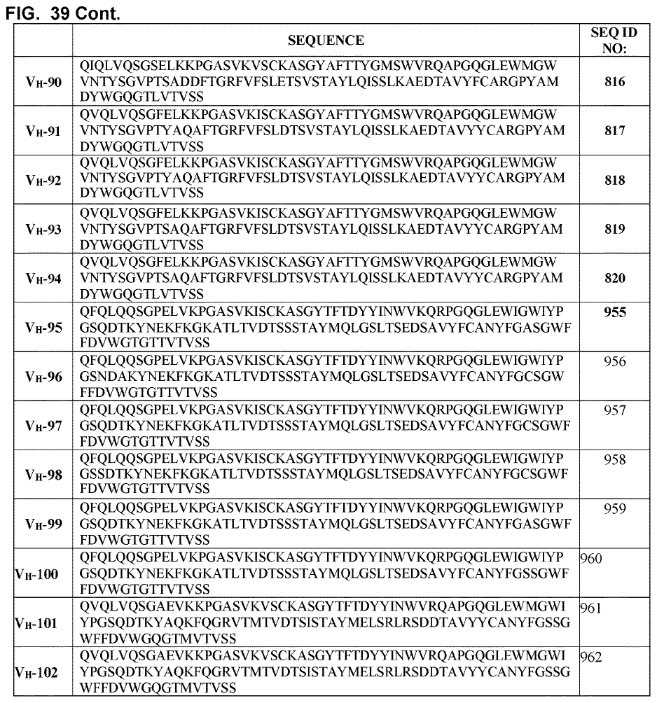

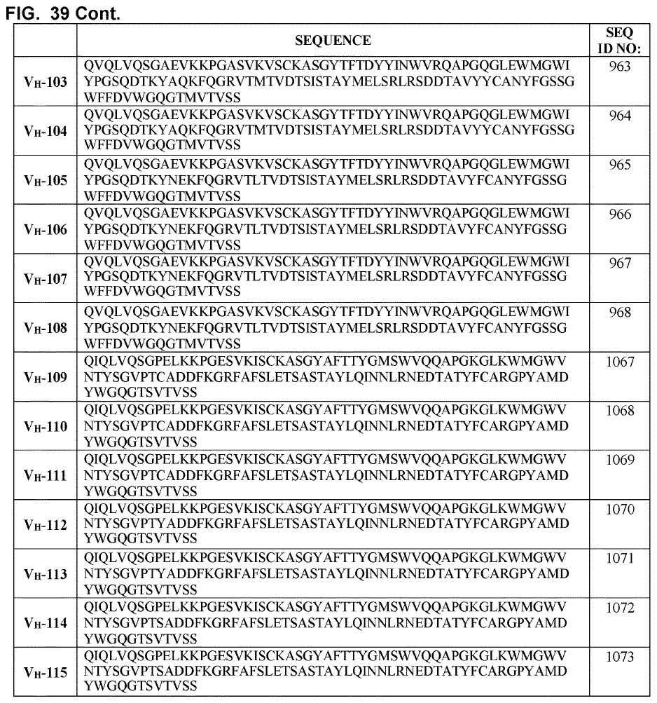

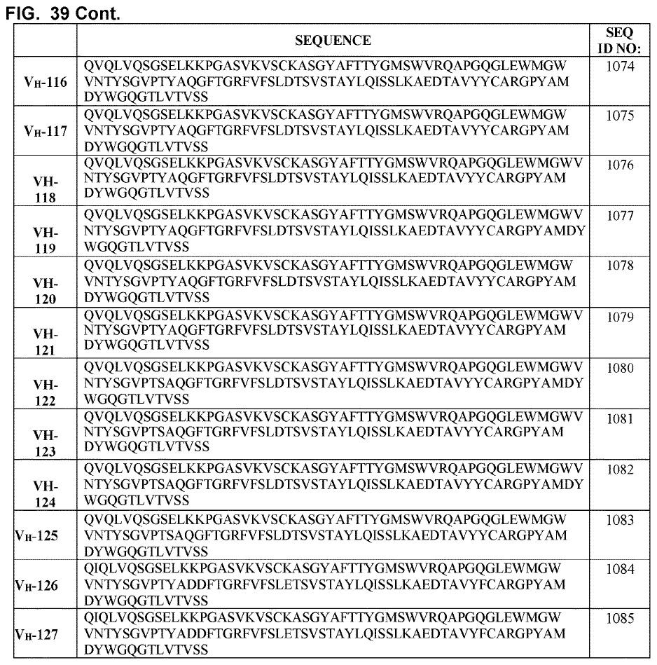

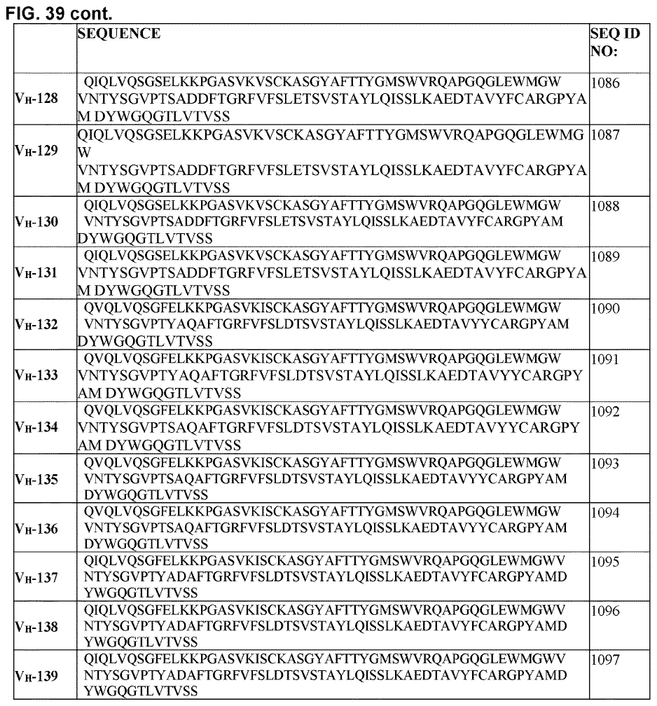

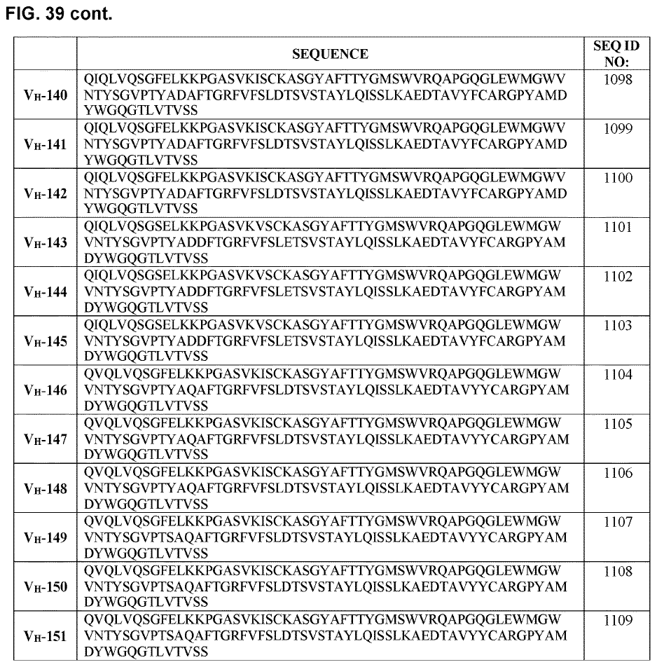

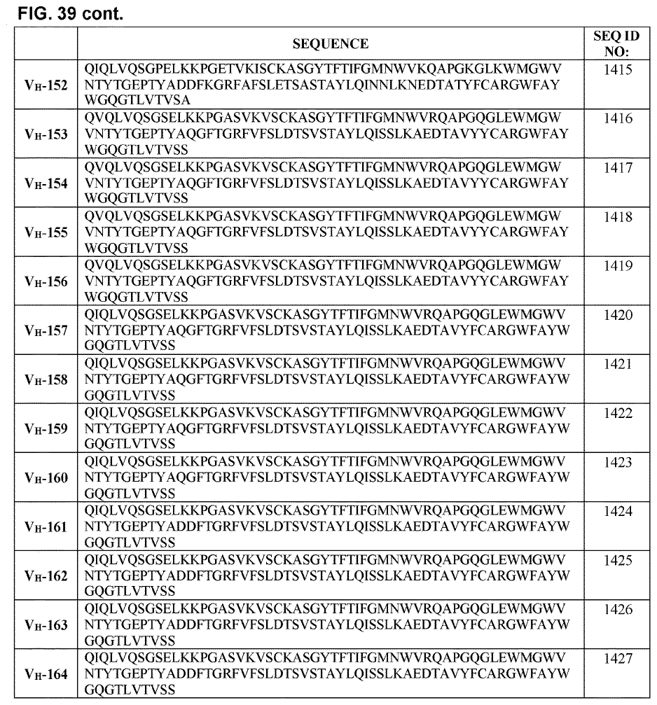

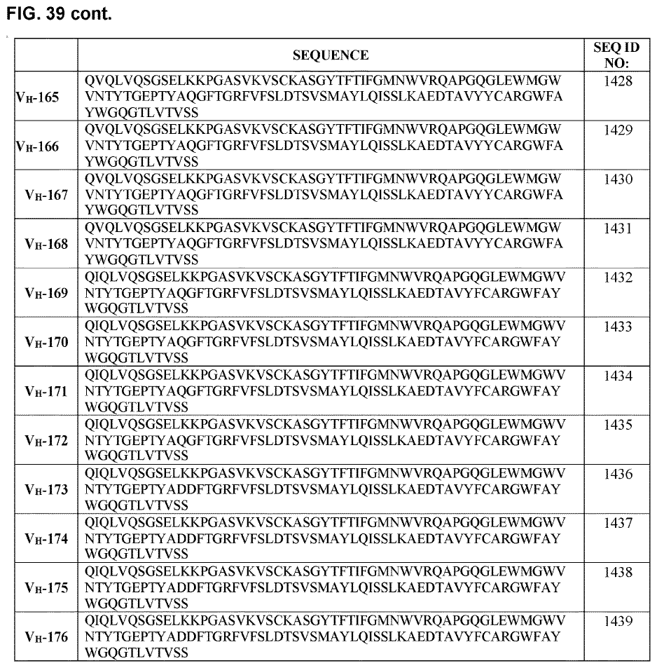

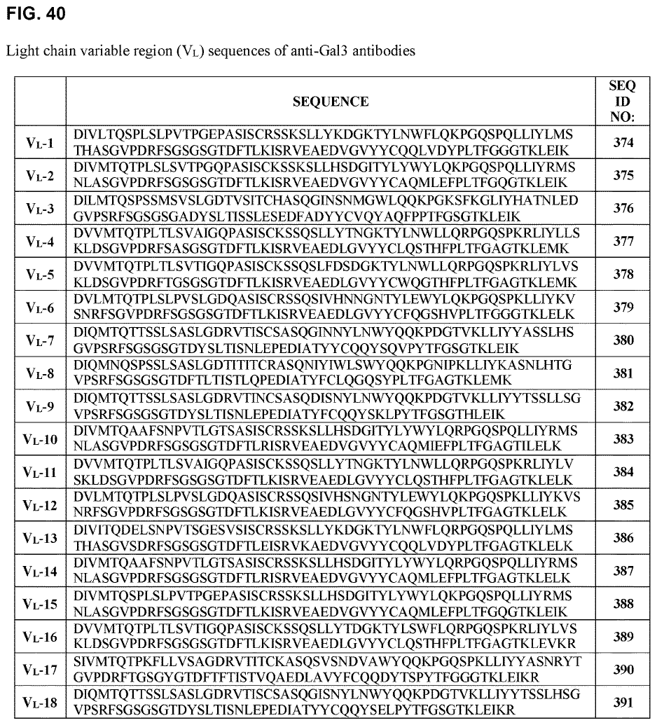

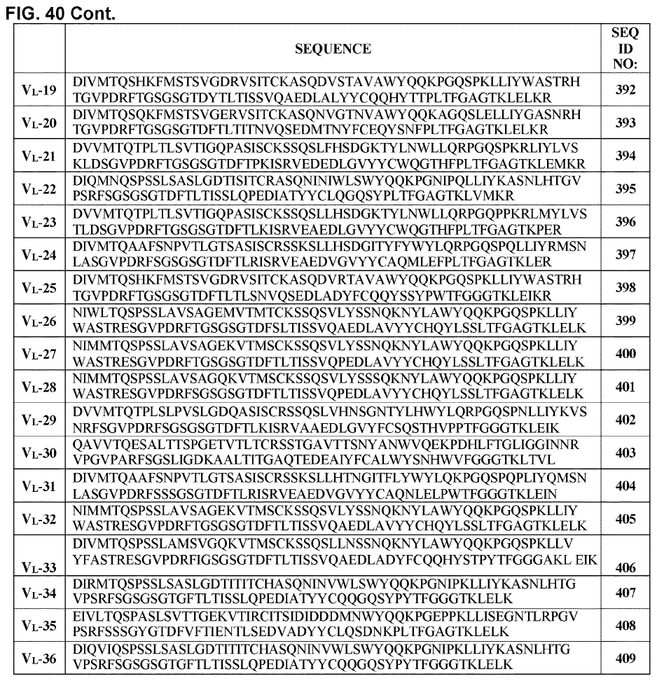

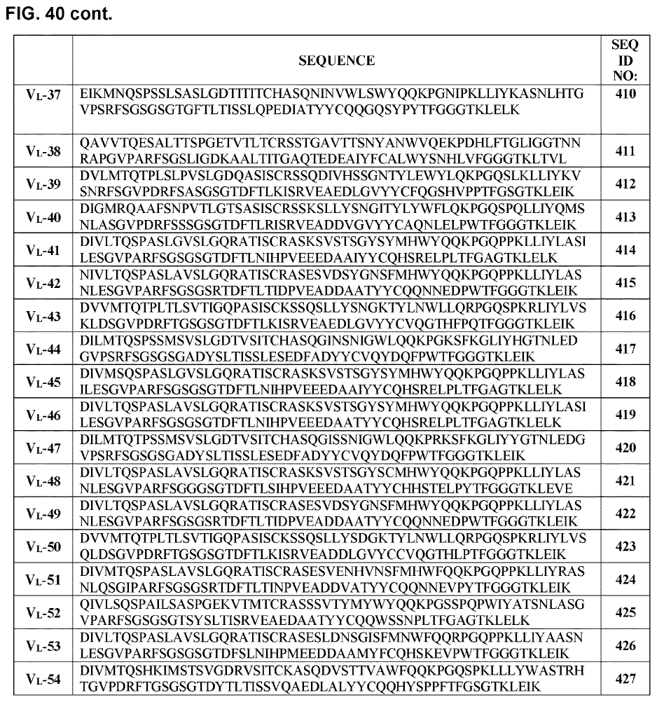

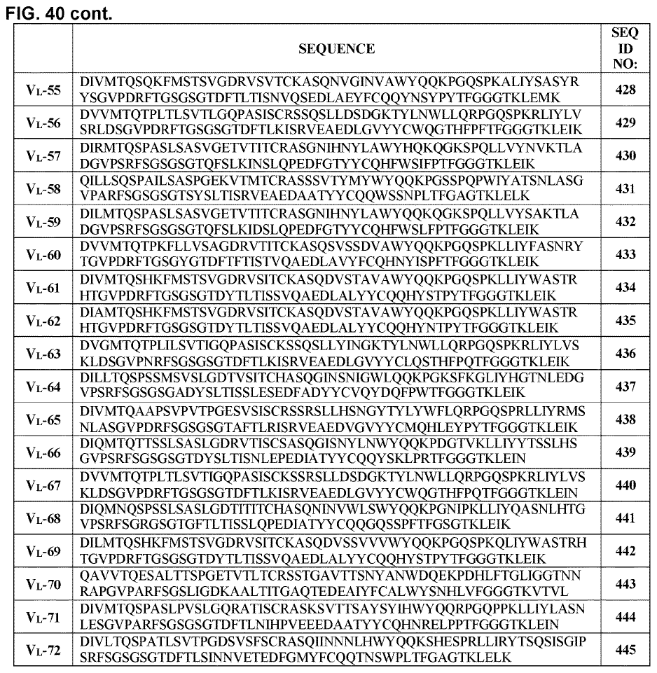

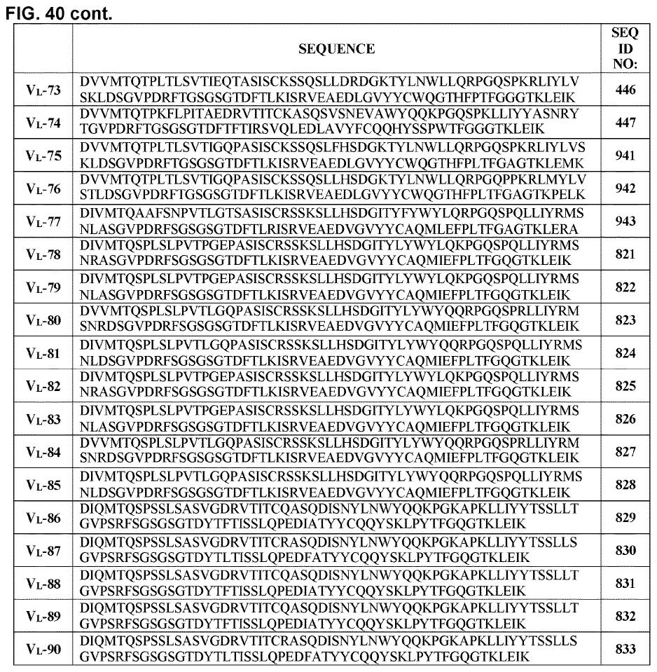

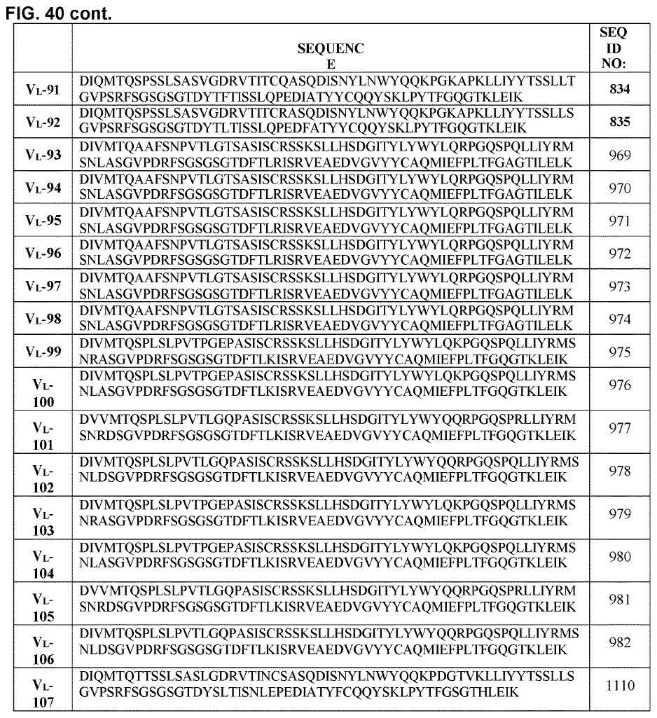

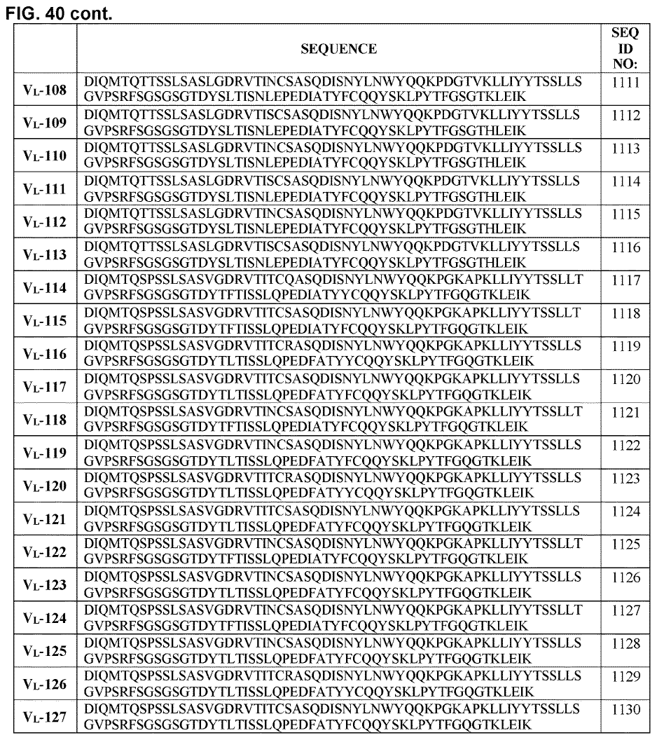

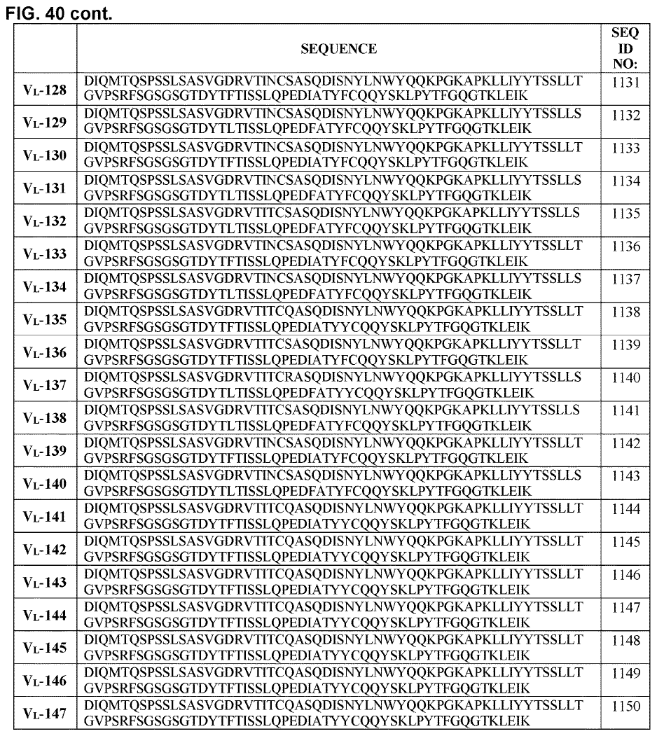

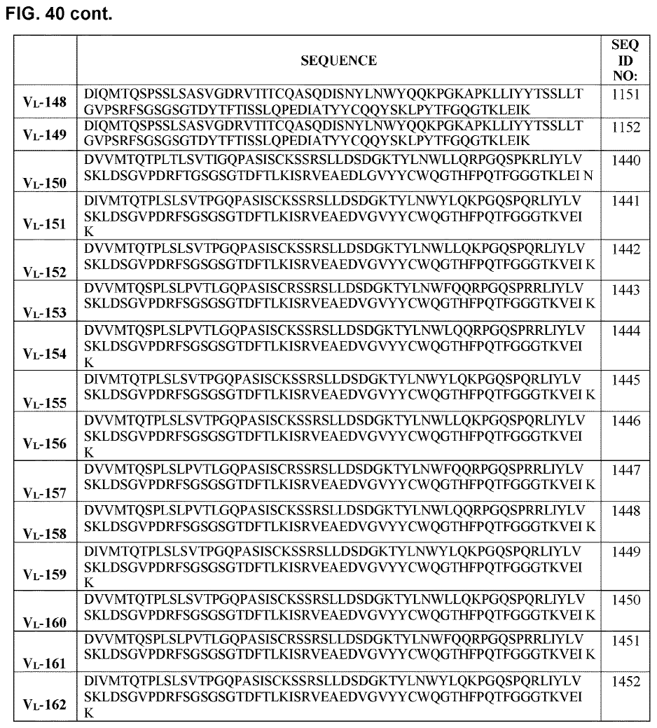

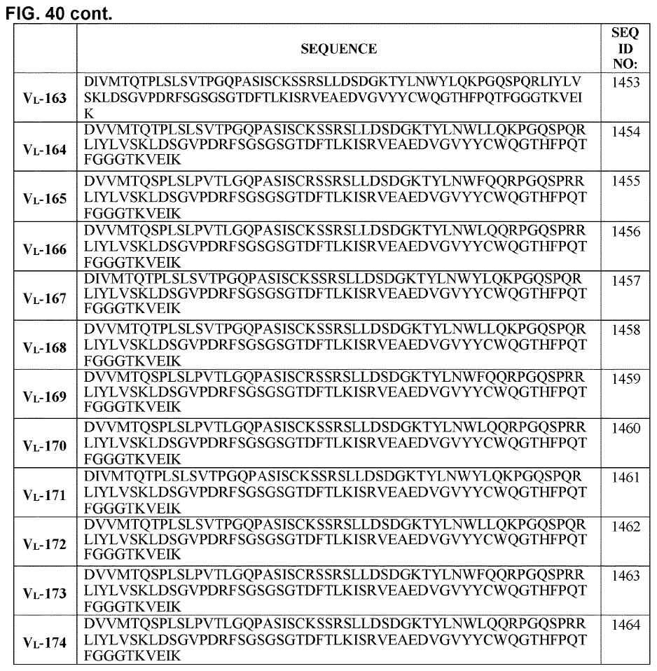

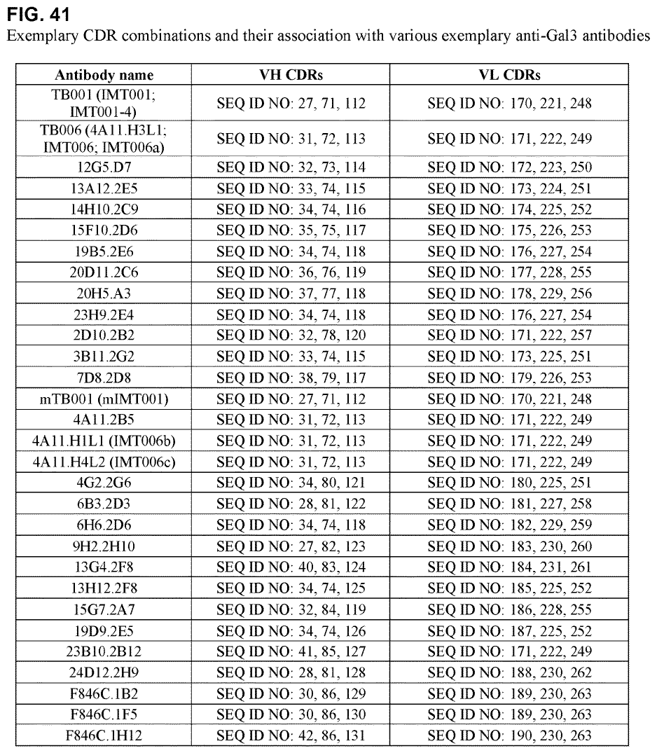

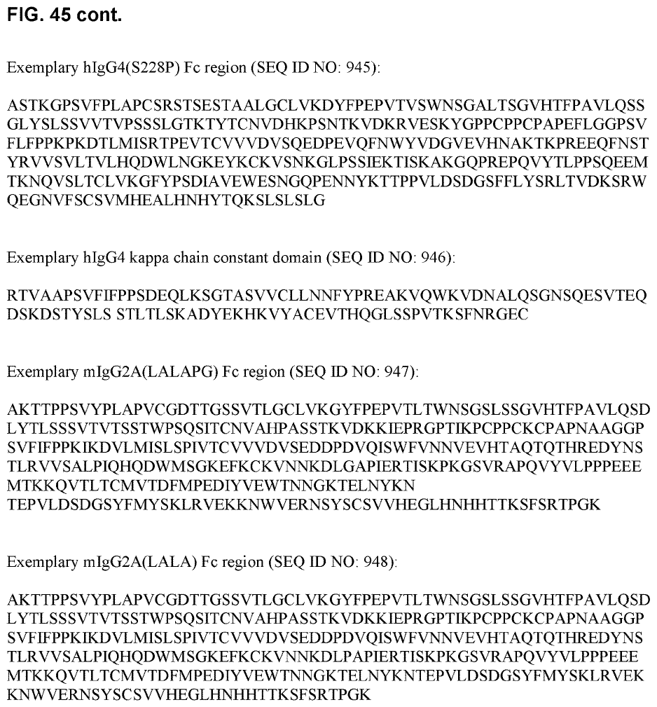

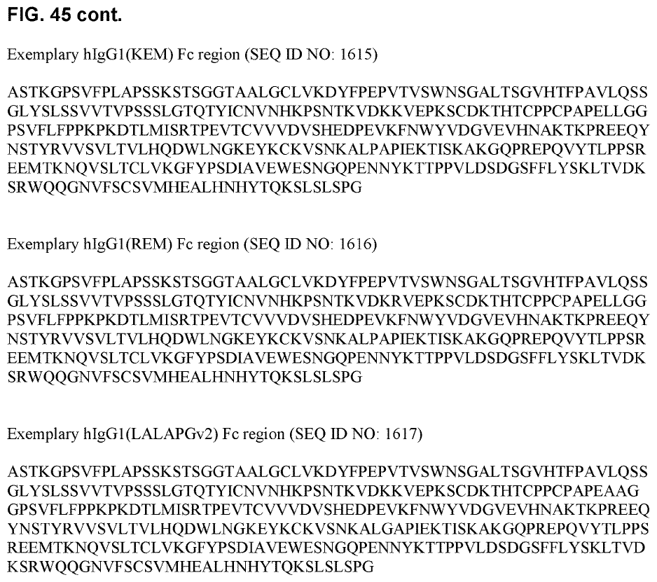

In some embodiments, the anti-Gal3 antibodies or binding fragments thereof comprise a heavy chain variable region and a light chain variable region. In some embodiments, the heavy chain variable region is paired with an IgG4 heavy chain constant domain or an IgG2 heavy chain constant domain. In some embodiments, the IgG4 heavy chain constant domain or IgG2 heavy chain constant domain are human or murine. In some embodiments, the IgG4 heavy chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 931. In some embodiments, the IgG4 heavy chain constant domain is an S228P mutant. In some embodiments, the IgG2 heavy chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 933 or SEQ ID NO: 934. In some embodiments, the IgG2 heavy chain constant domain is a LALAPG or a LALA mutant. In some embodiments, the light chain variable region is paired with an IgG4 kappa chain constant domain. In some embodiments, the IgG4 kappa chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 932. Exemplary heavy chain and light chain constant domains can be found in FIG. 45. In some embodiments, the light chain variable region and/or heavy chain variable region may be selected from those depicted in FIGS. 39 and 40 and/or the combinations of light chain variable region and heavy chain variable region as depicted in FIG. 45. In some embodiments, the light chain variable region and/or heavy chain variable regions comprise one or more CDRs depicted in FIG. 12A-C, 13A-C and/or the combinations of CDRs depicted in FIG. 41.

In some embodiments, the antibody or binding fragment thereof is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of at least one of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of at least one of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

In some embodiments, the antibody or binding fragment thereof comprises a sequence (e.g. CDR, VL, VH, LC, HC) having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of at least one of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of at least one of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

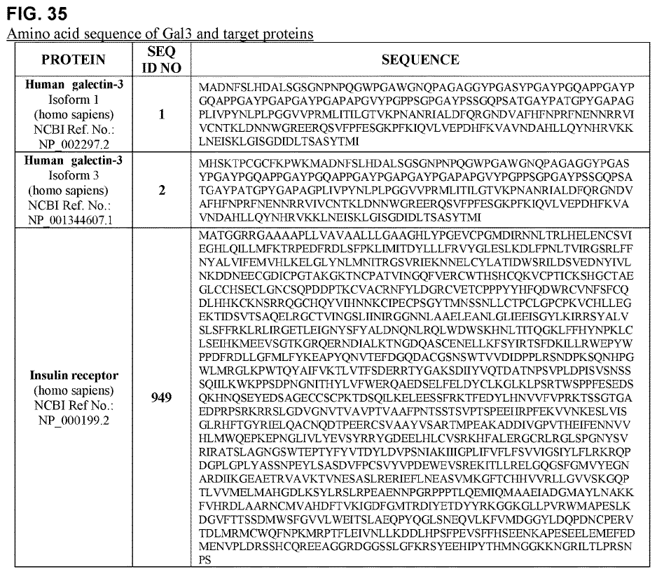

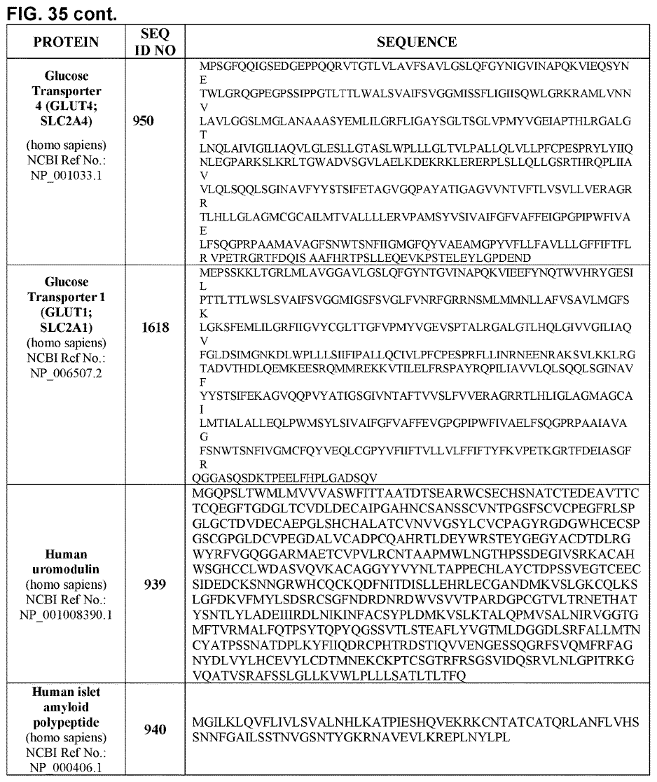

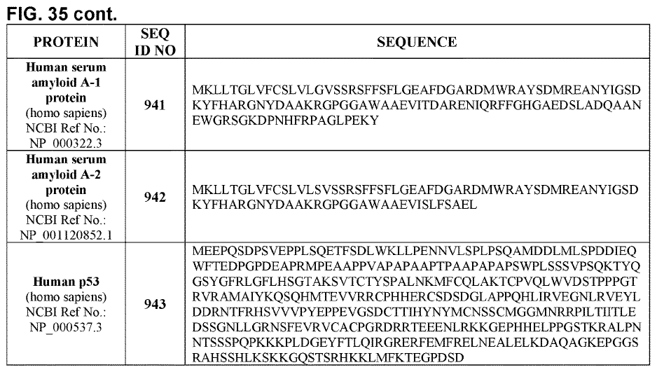

In some embodiments, the anti-Gal3 antibody or binding fragment thereof binds to specific epitopes within a Gal3 protein. In some embodiments, the anti-Gal3 antibody or binding fragment thereof binds to a specific epitope within a Gal3 protein having an amino acid sequence according to SEQ ID NO: 1-2, provided in FIG. 35.

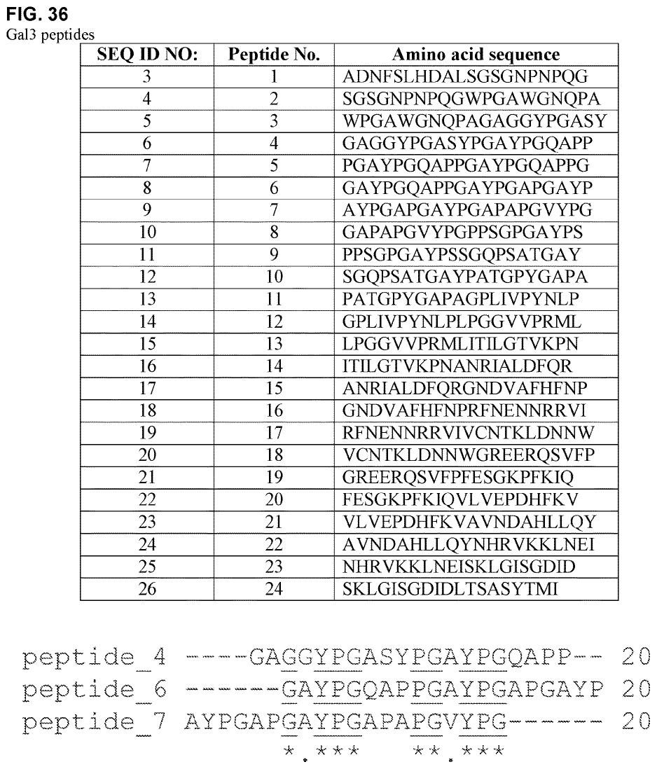

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within a peptide illustrated in FIG. 36 (SEQ ID NOs: 3-26).

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 1-20 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 31-50 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 51-70 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 61-80 of SEQ ID NO: 1-2.

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 1 (SEQ ID NO: 3), Peptide 4 (SEQ ID NO: 6), Peptide 6 (SEQ ID NO: 8), or Peptide 7 (SEQ ID NO: 9). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 1 (SEQ ID NO: 3). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 4 (SEQ ID NO: 6). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 6 (SEQ ID NO: 8). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 7 (SEQ ID NO: 9). In some embodiments, the anti-Gal3 antibody or binding fragment thereof binds to an epitope present within a region of Gal3 defined by Peptide 1 (SEQ ID NO: 3). In some embodiments, the anti-Gal3 antibody or binding fragment thereof binds to an epitope present within a region of Gal3 defined by Peptide 4 (SEQ ID NO: 6). In some embodiments, the anti-Gal3 antibody or binding fragment thereof binds to an epitope present within a region of Gal3 defined by Peptide 6 (SEQ ID NO: 8). In some embodiments, the anti-Gal3 antibody or binding fragment thereof binds to an epitope present within a region of Gal3 defined by Peptide 7 (SEQ ID NO: 9). In some embodiments, the antibody is one that binds to 1, 2, or all 3 of peptides 1, 6, and/or 7.

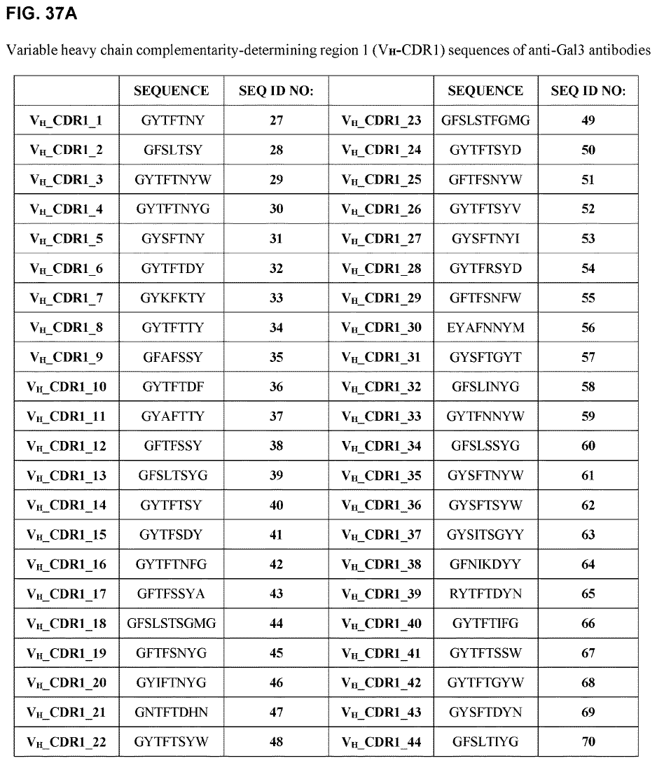

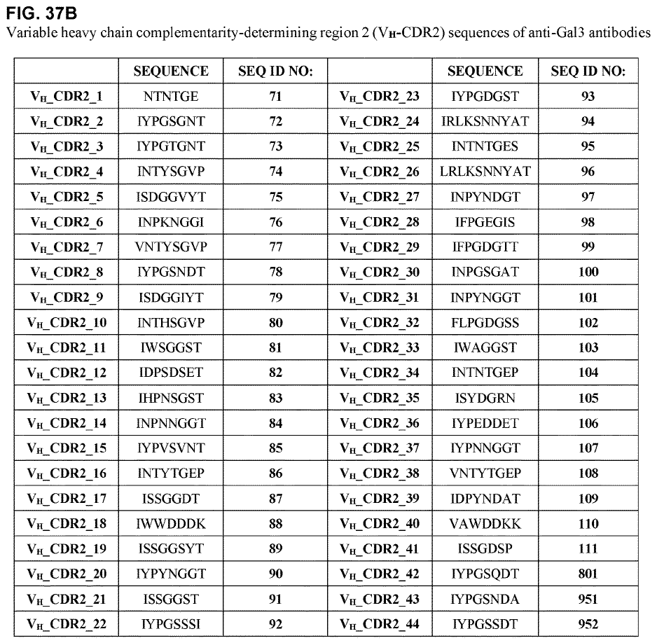

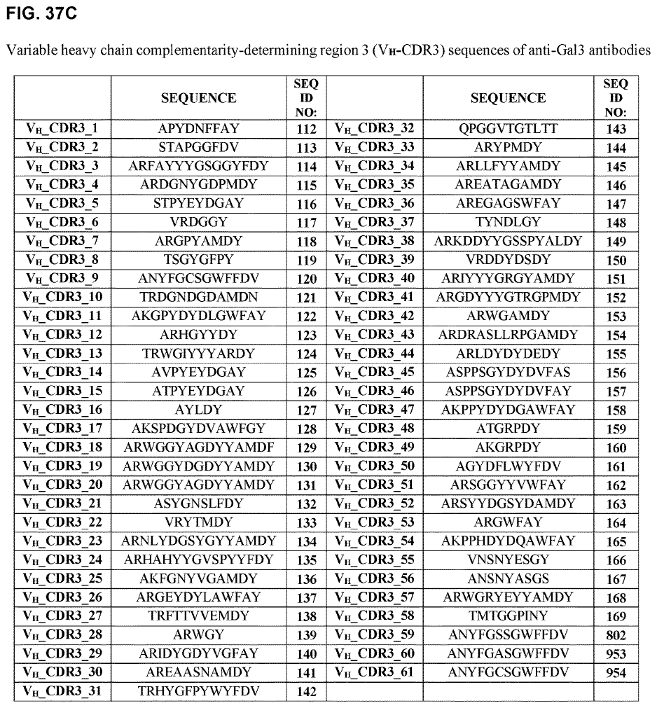

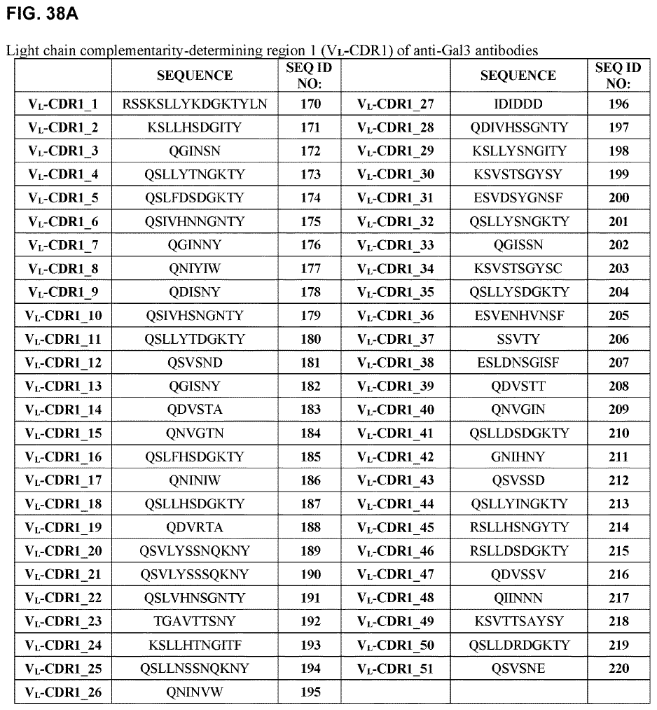

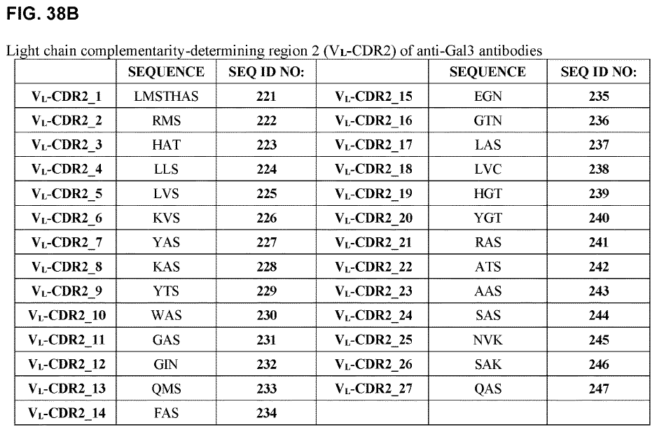

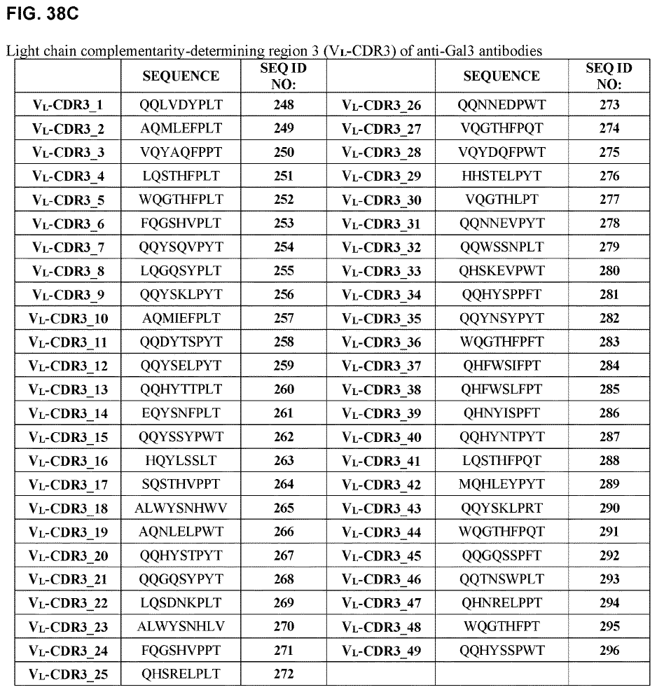

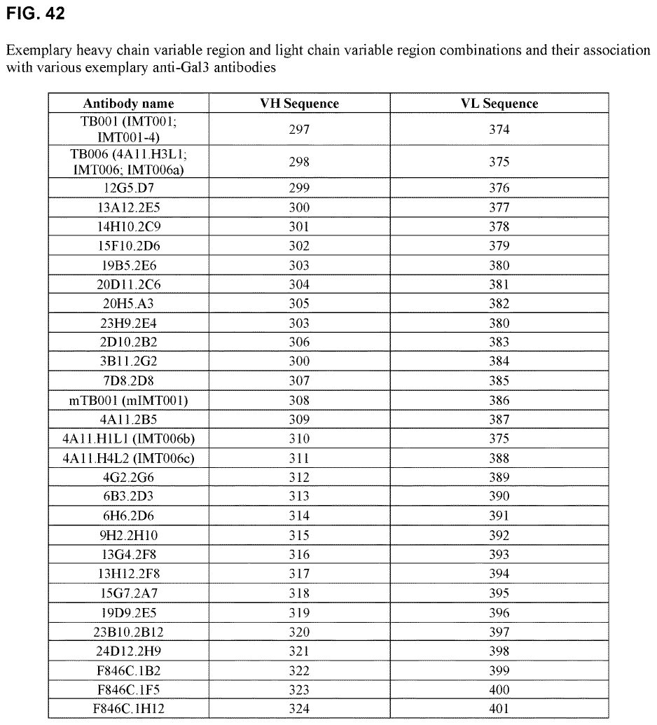

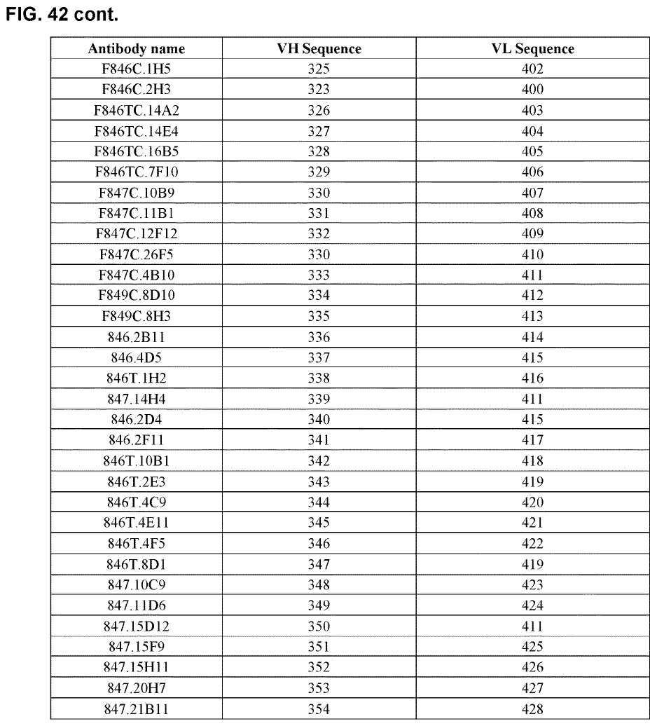

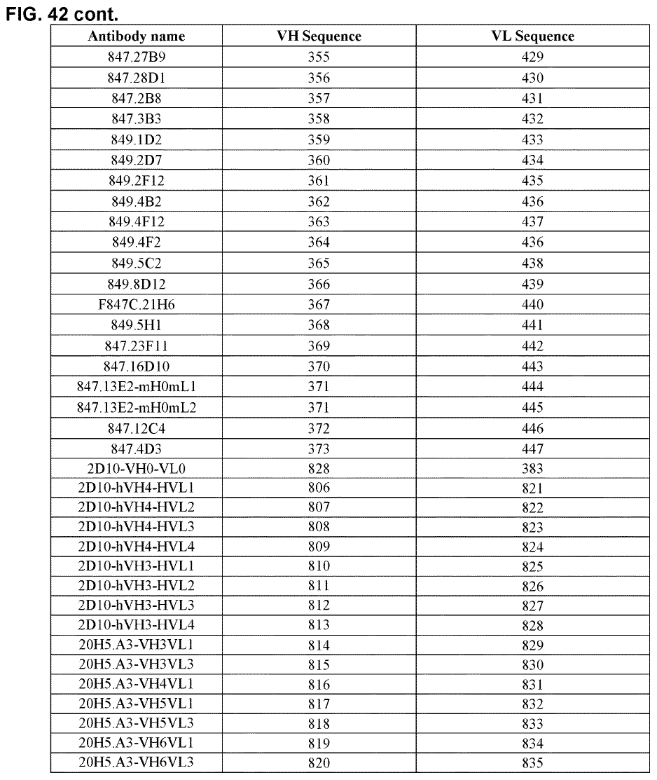

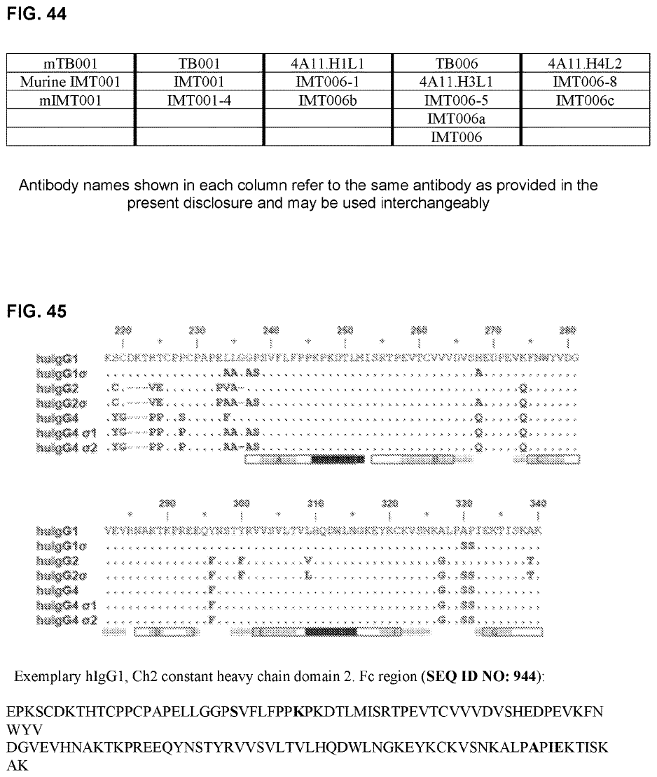

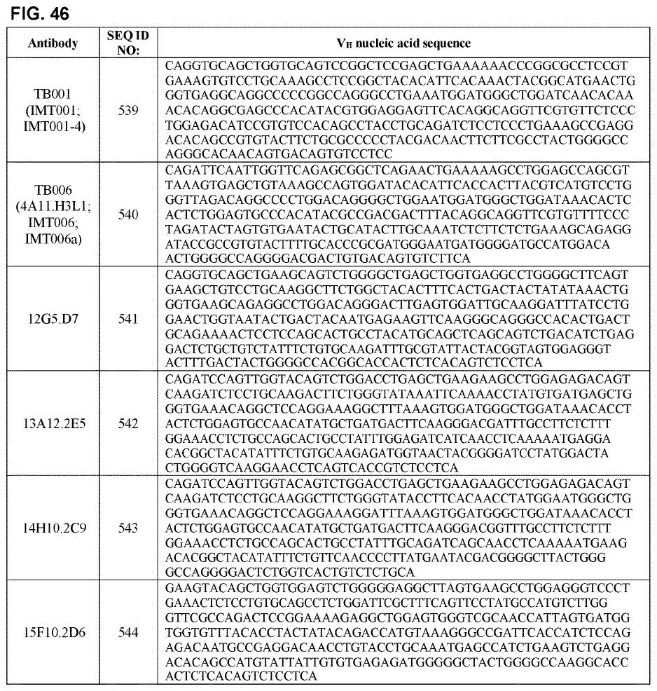

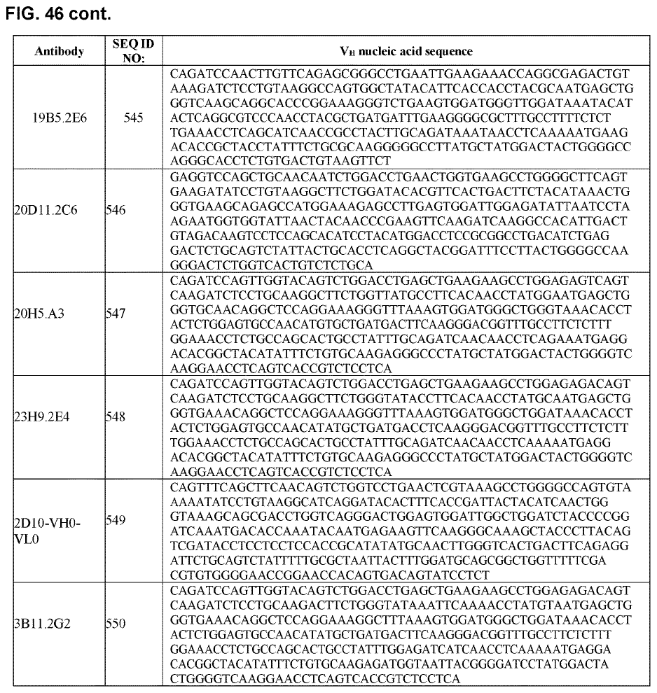

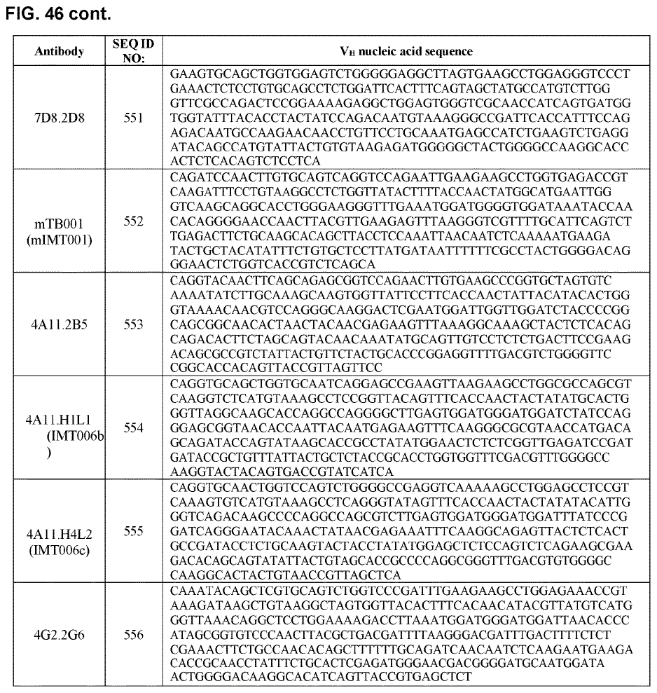

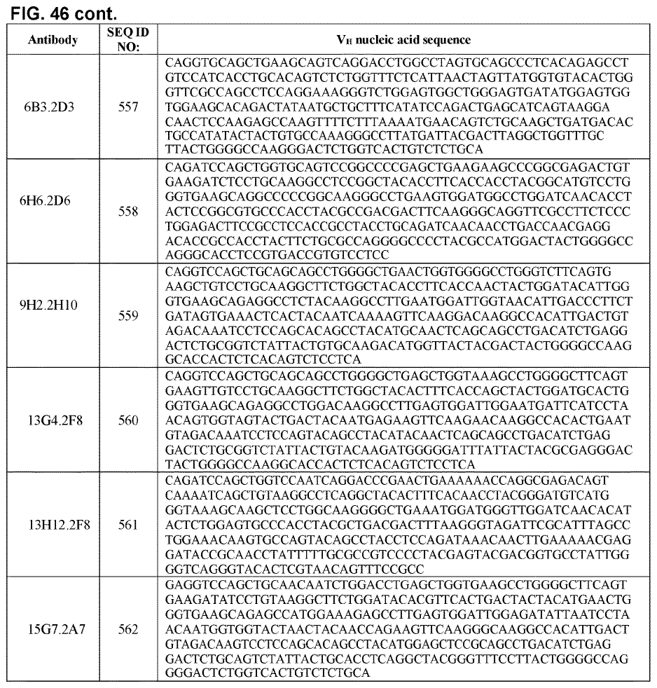

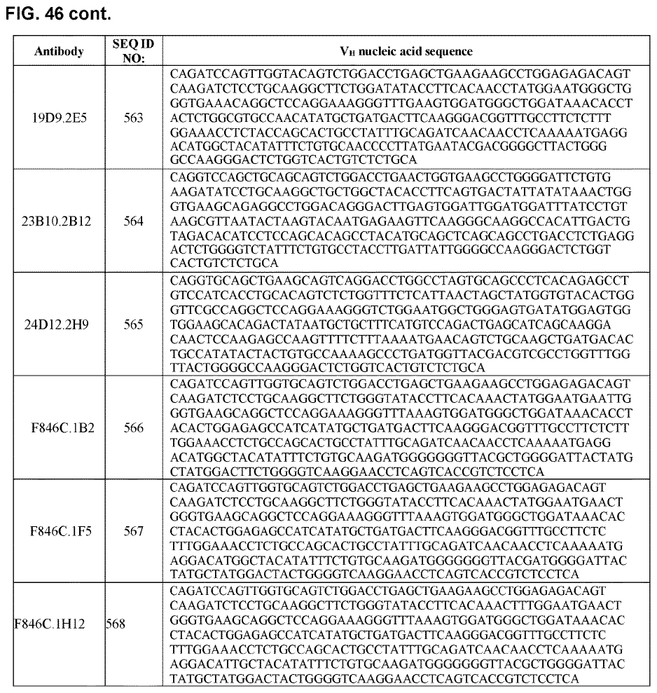

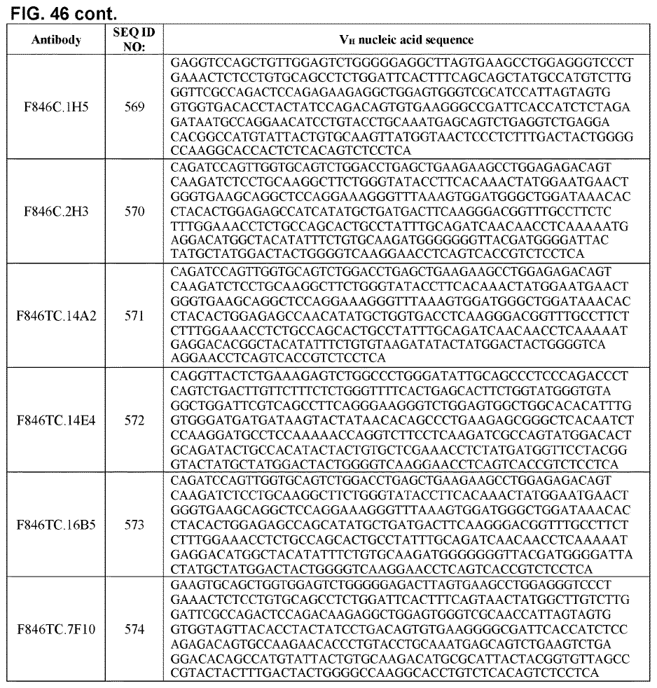

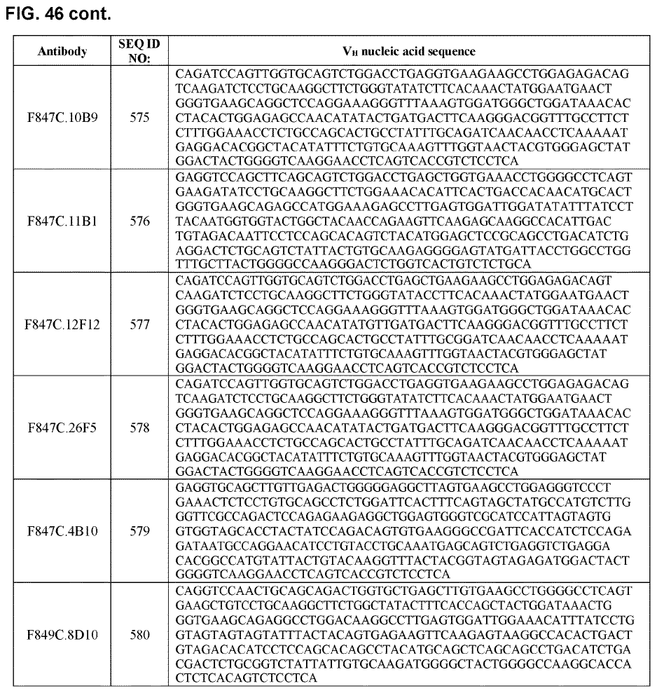

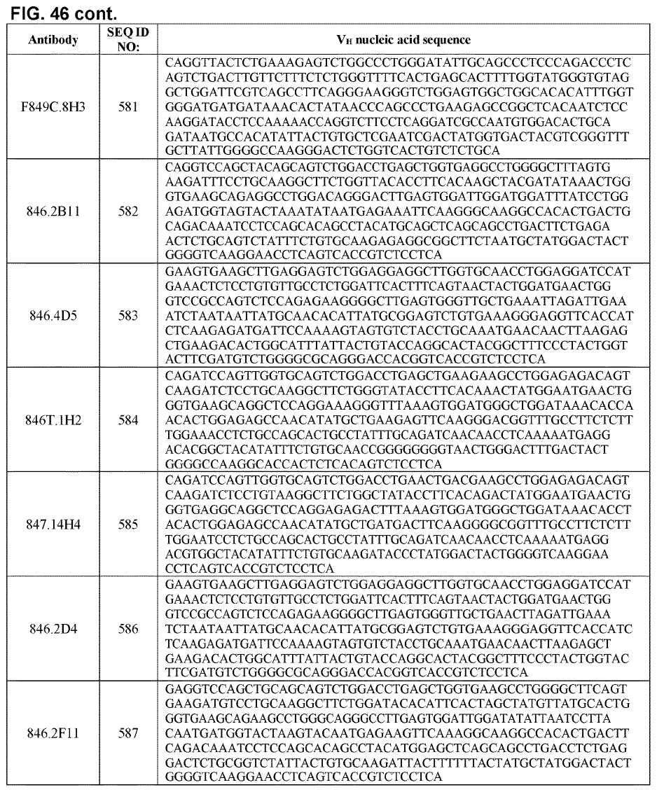

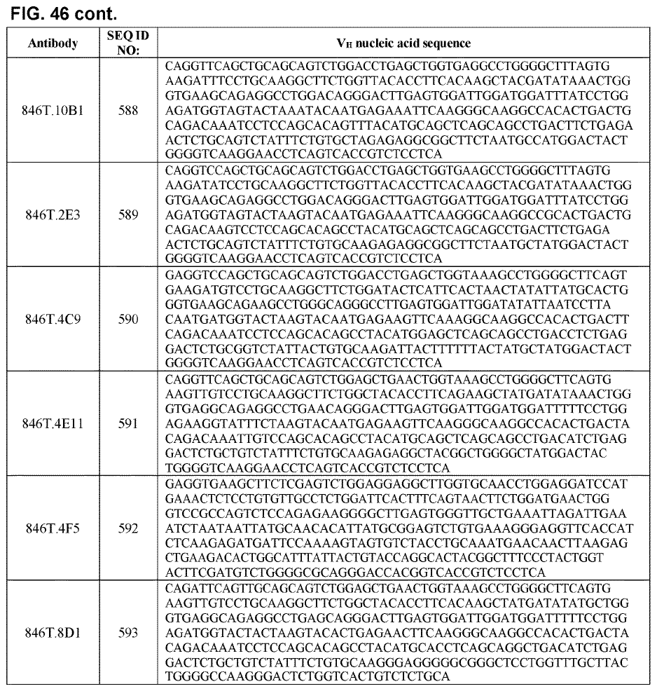

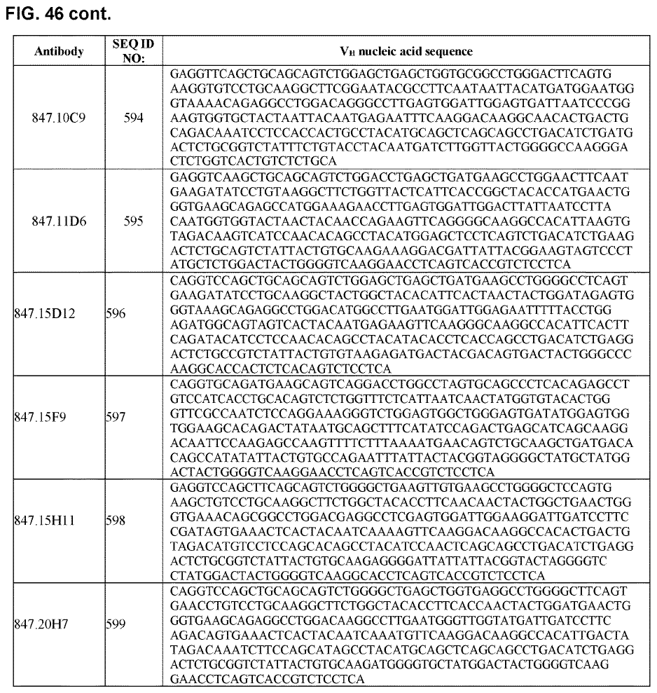

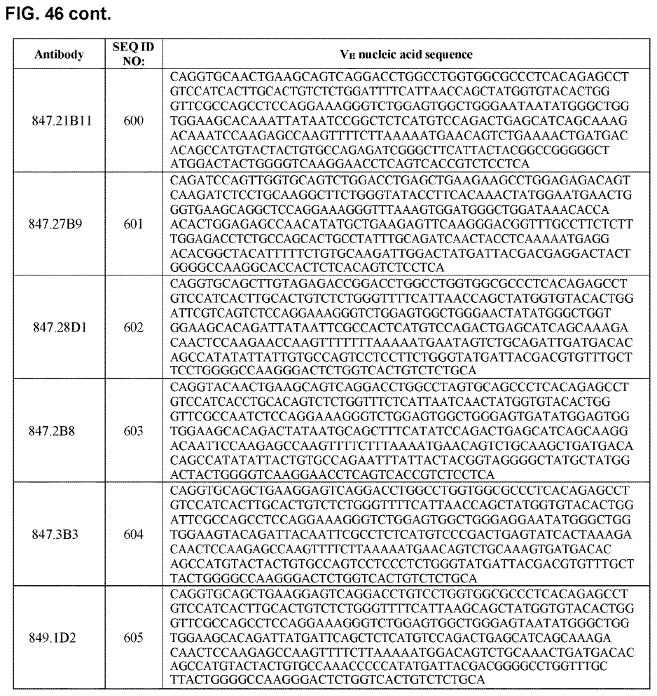

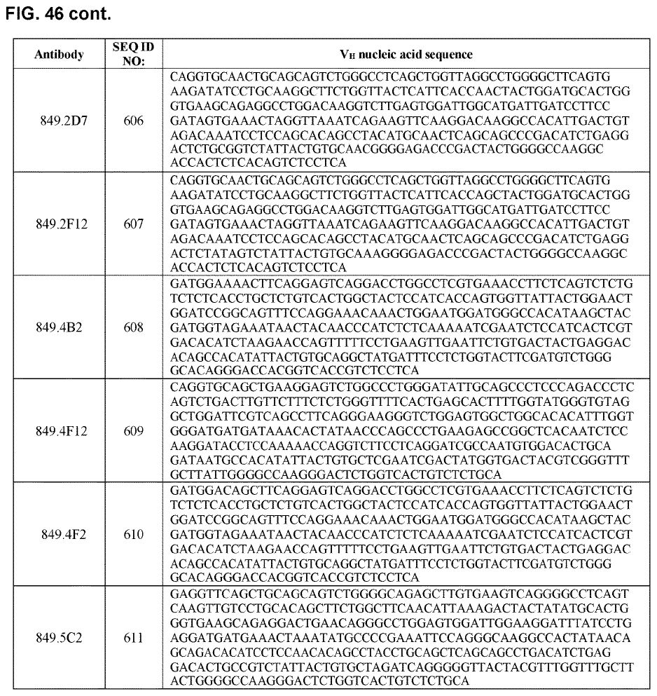

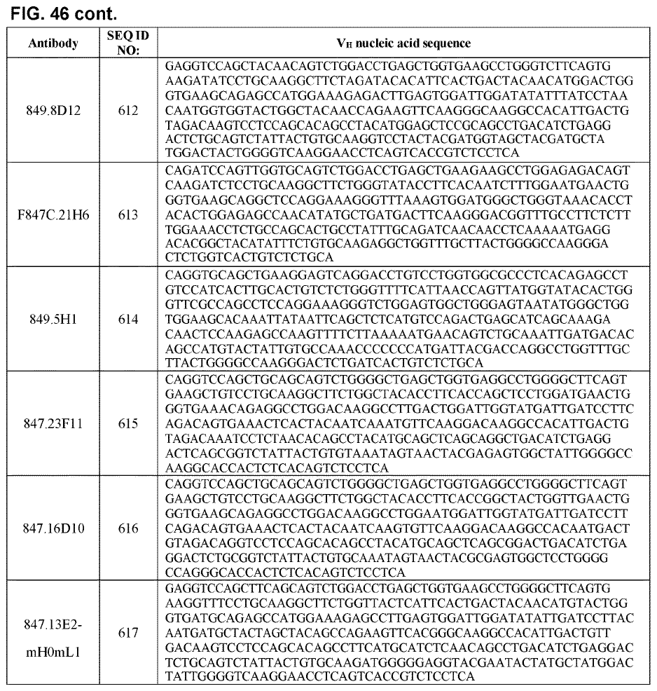

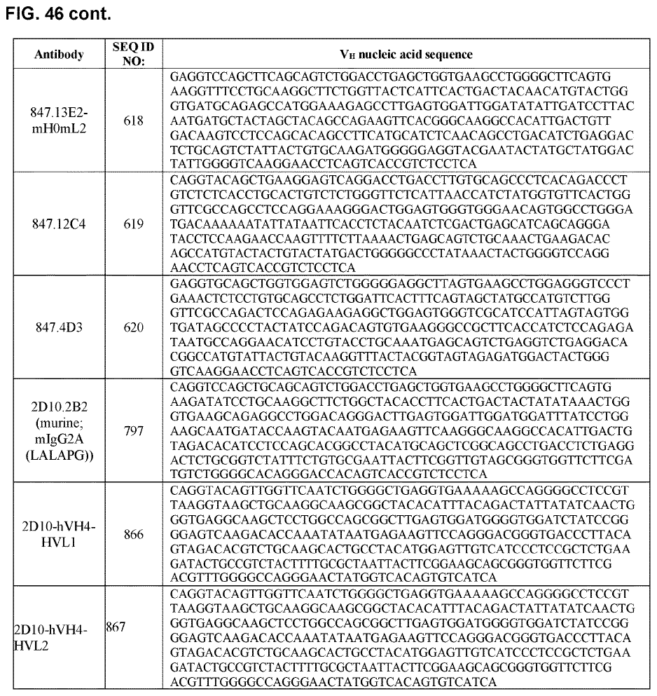

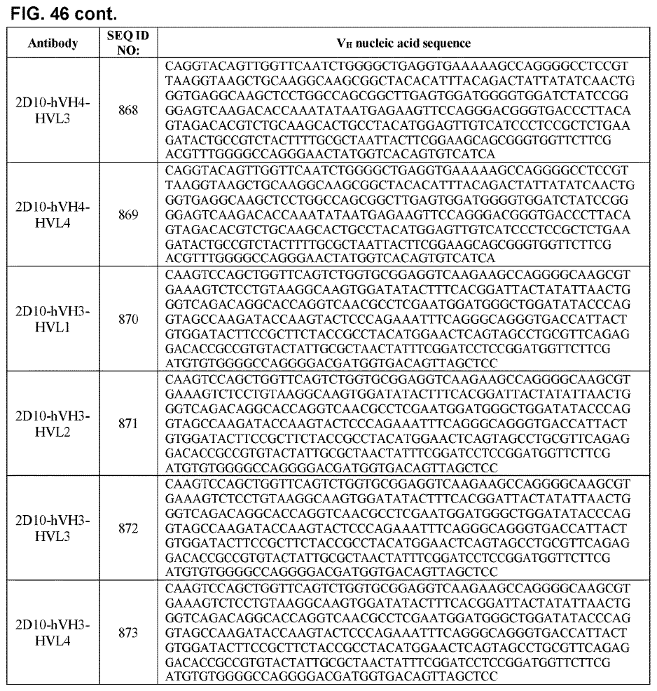

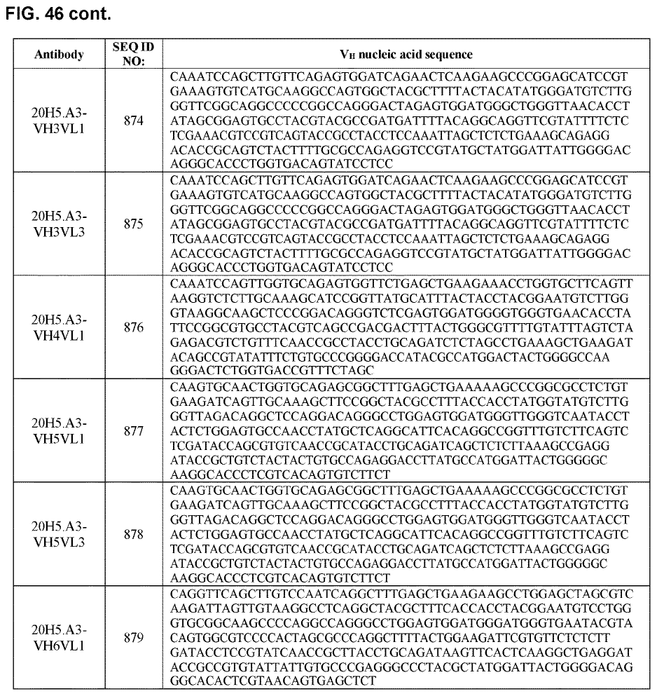

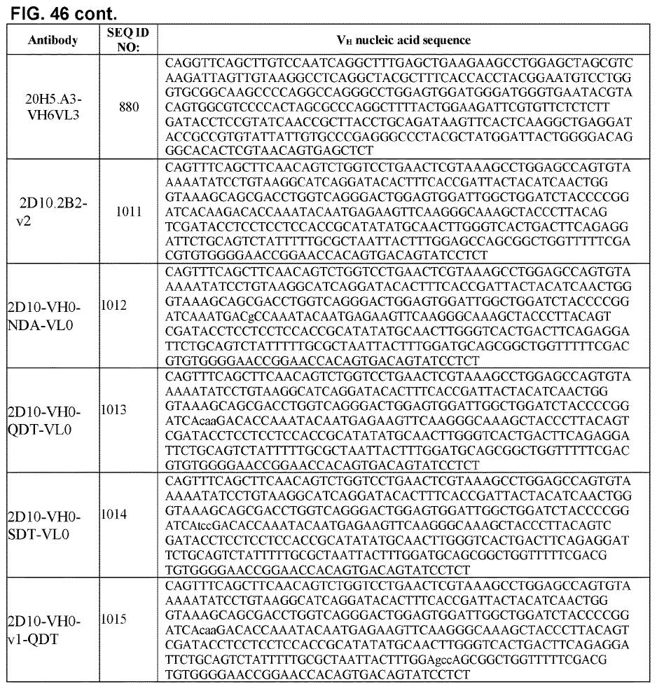

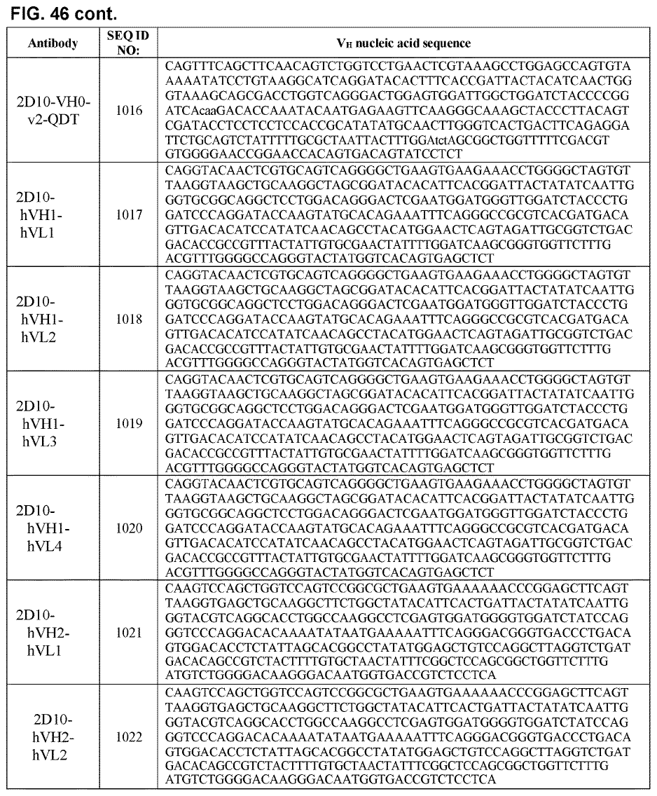

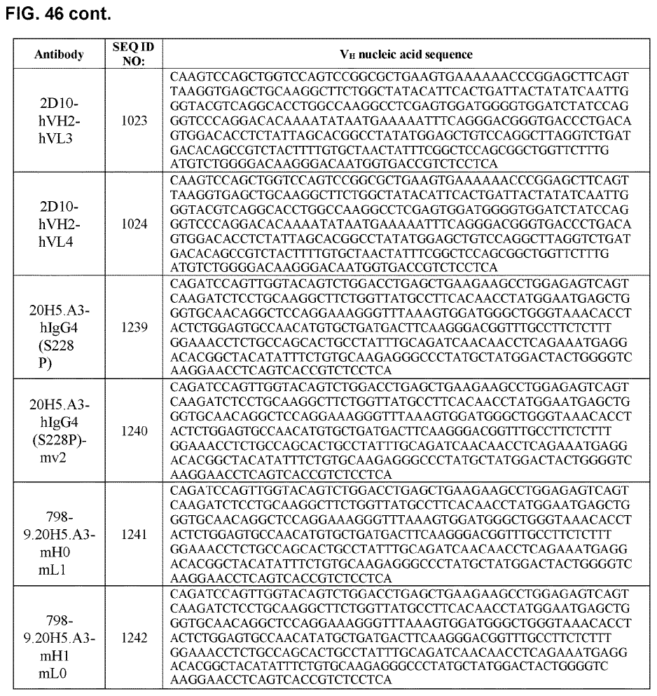

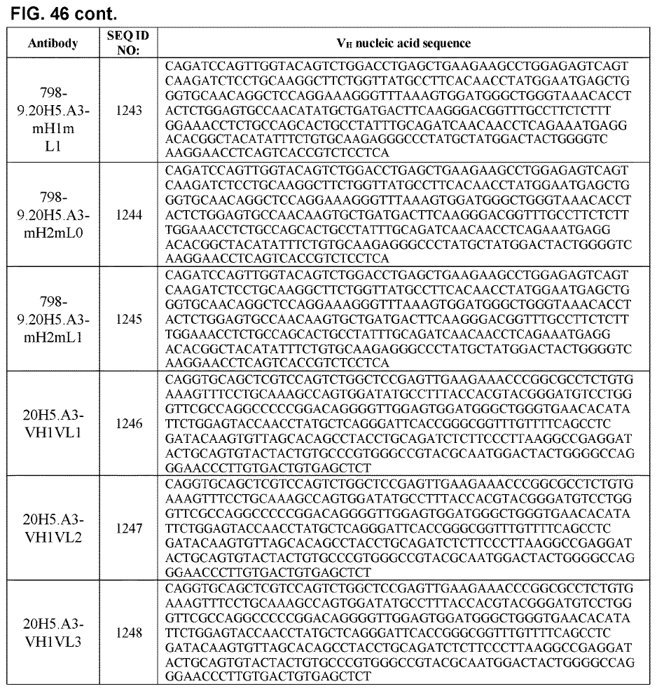

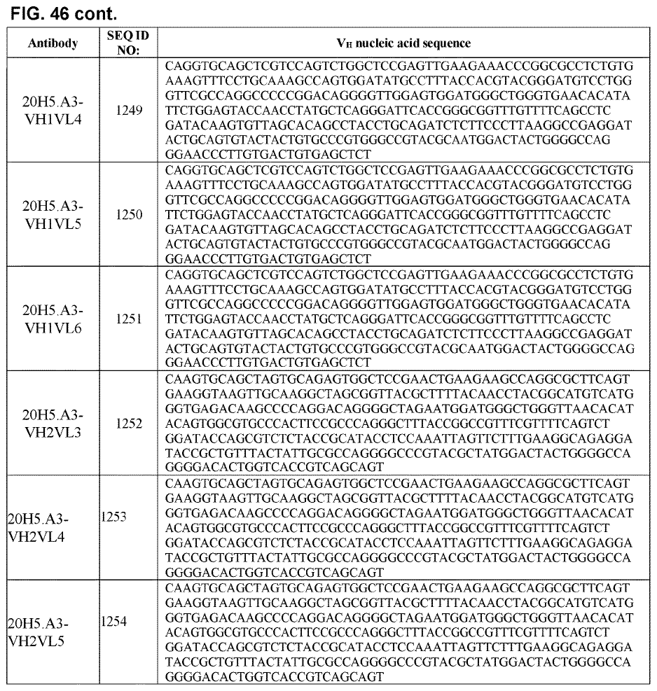

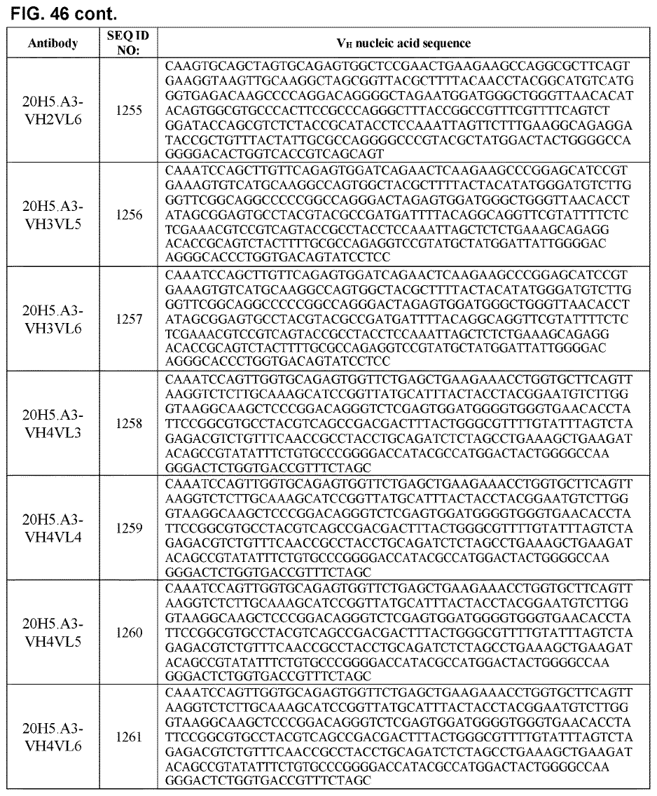

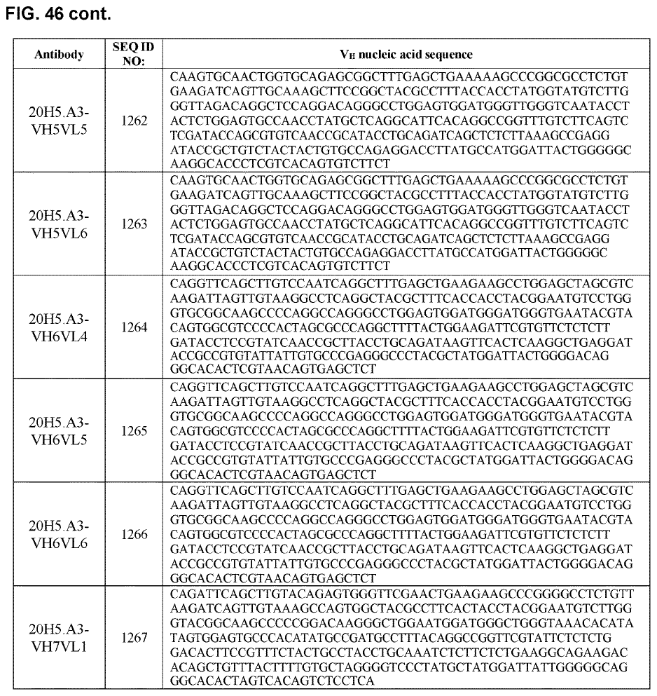

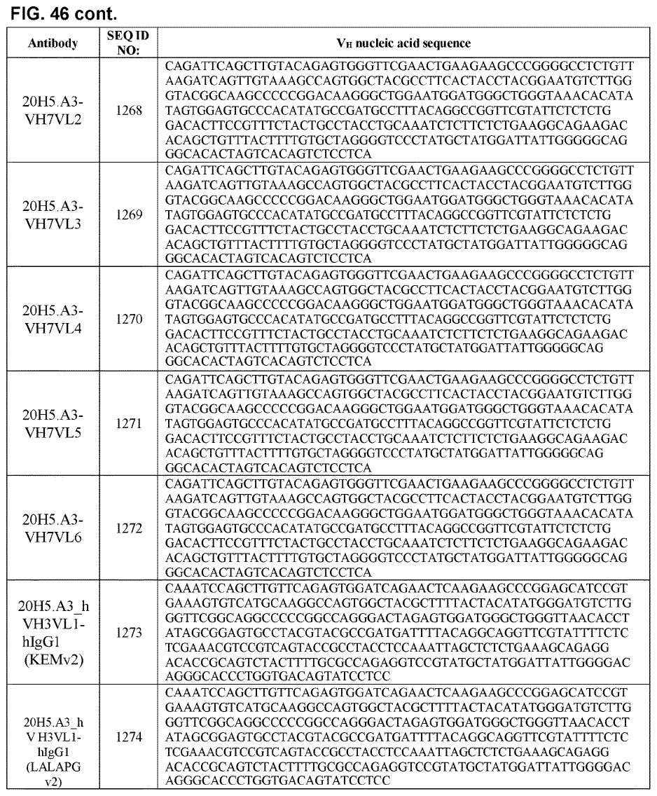

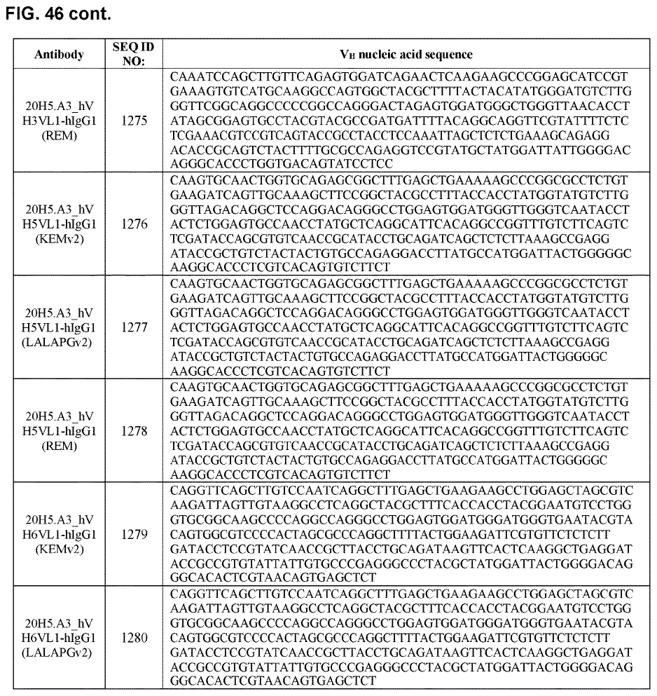

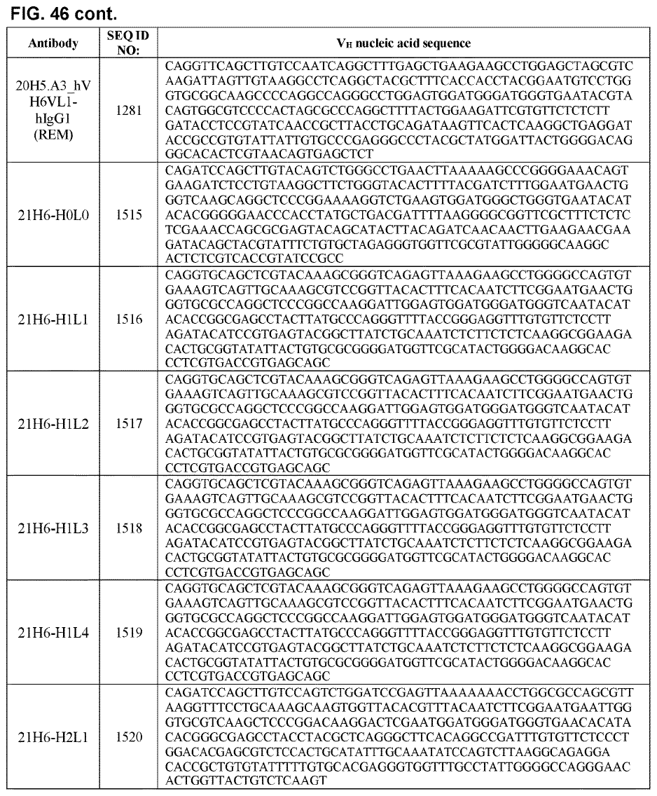

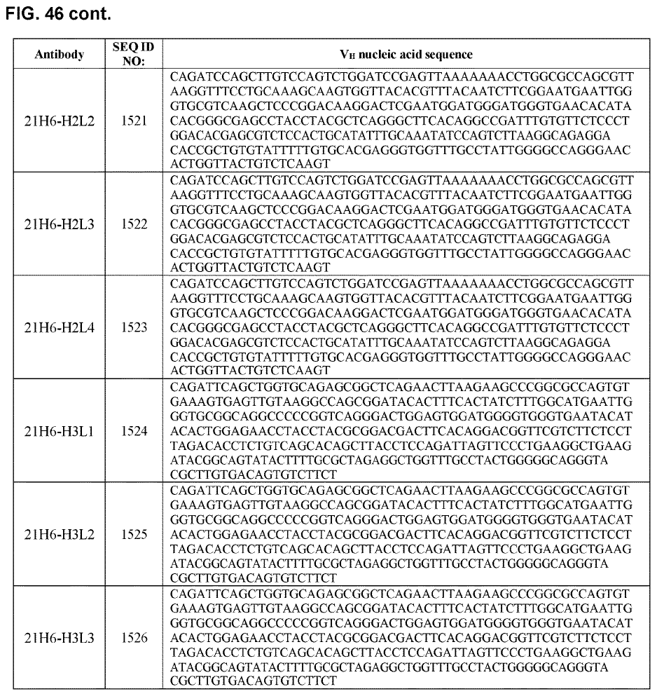

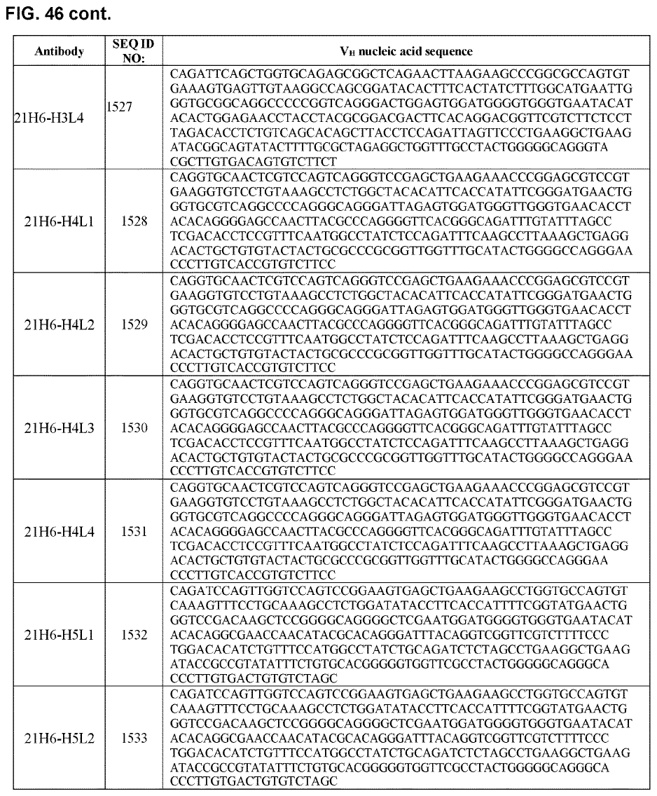

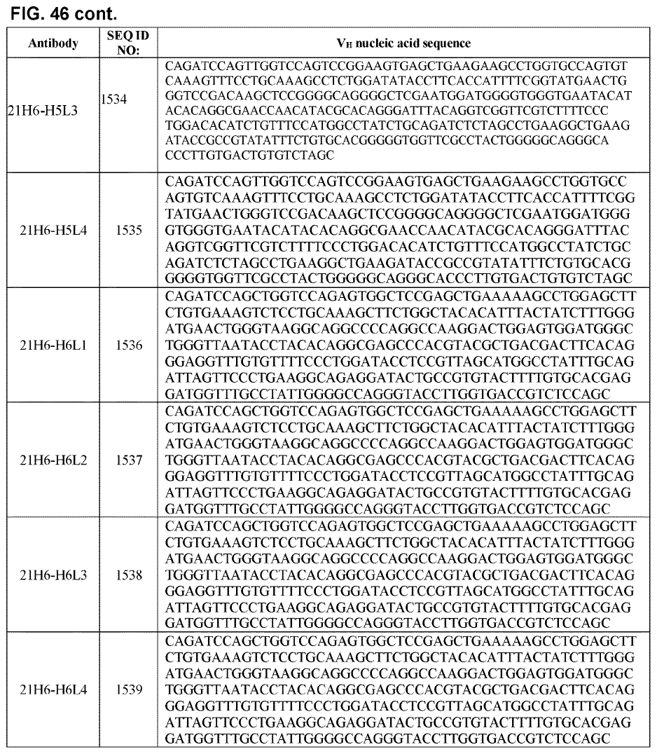

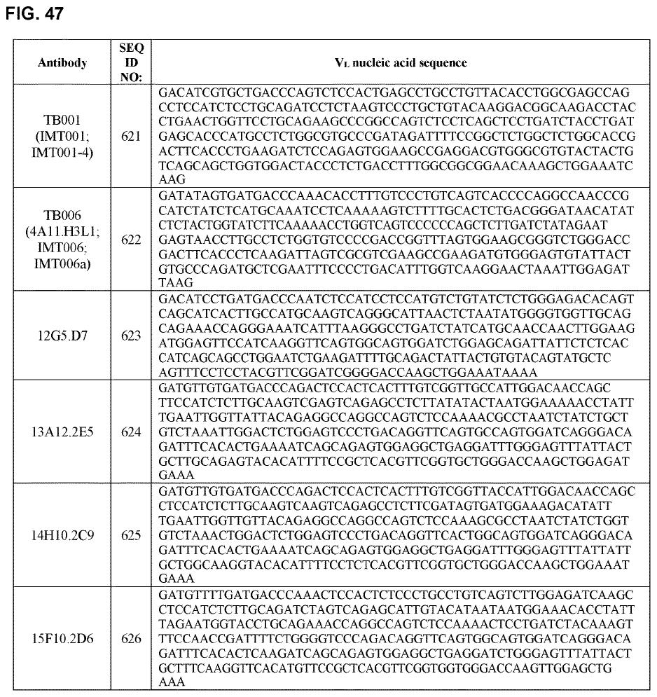

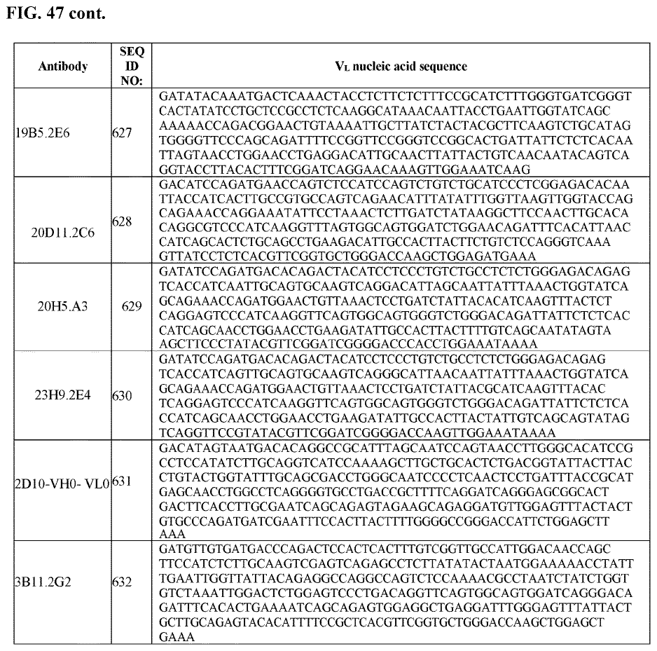

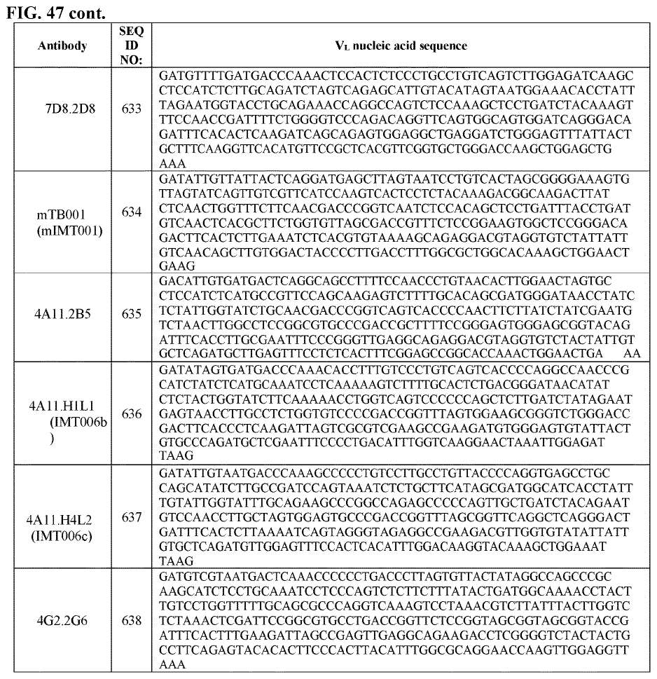

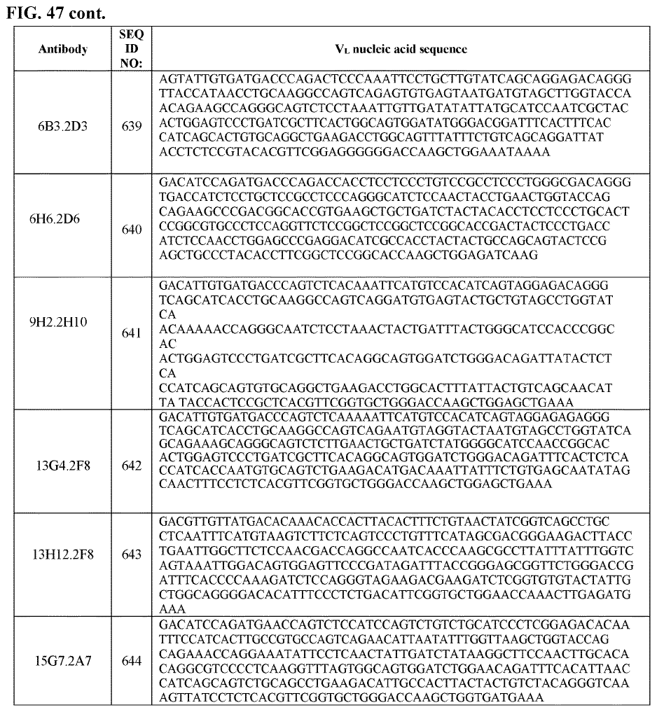

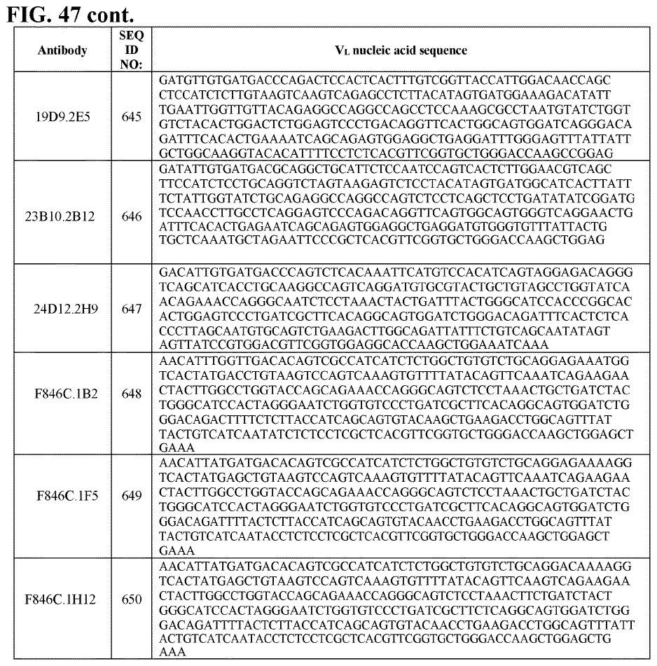

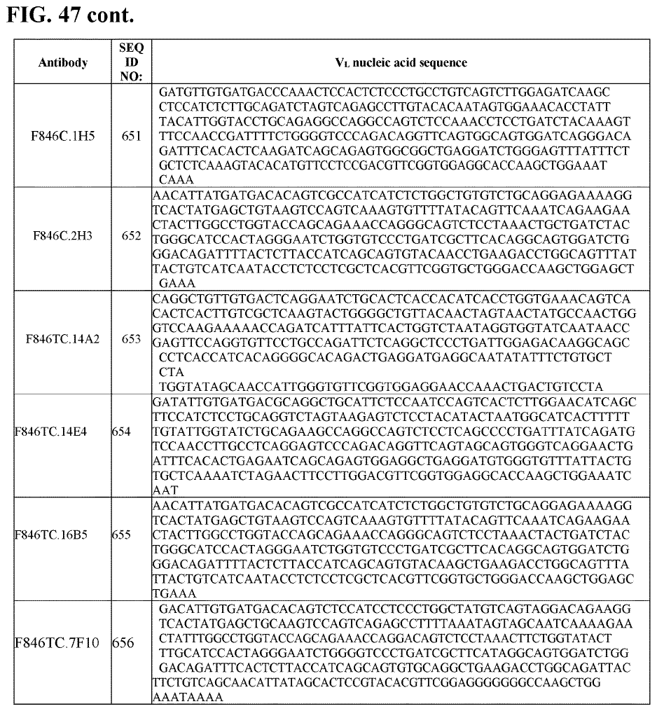

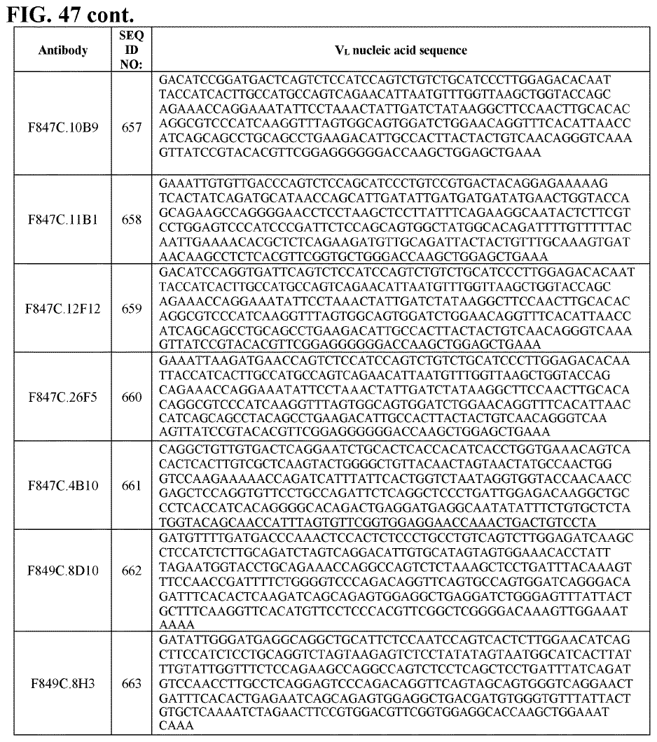

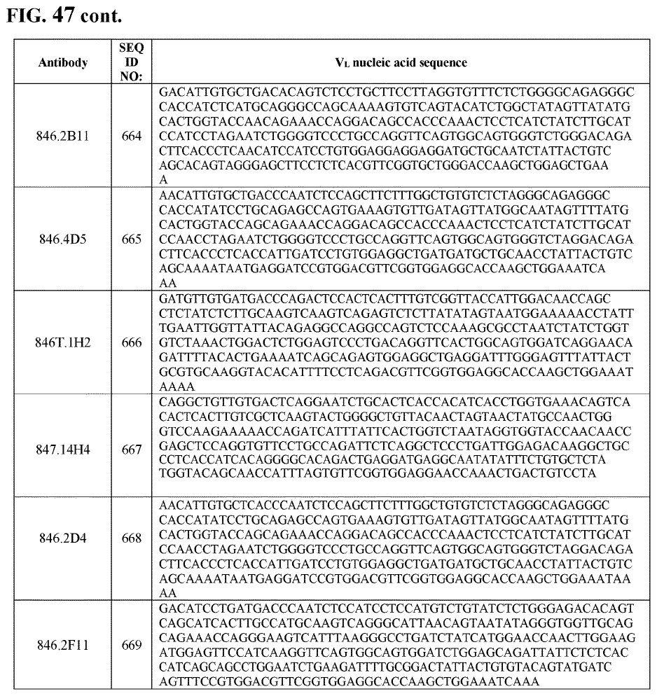

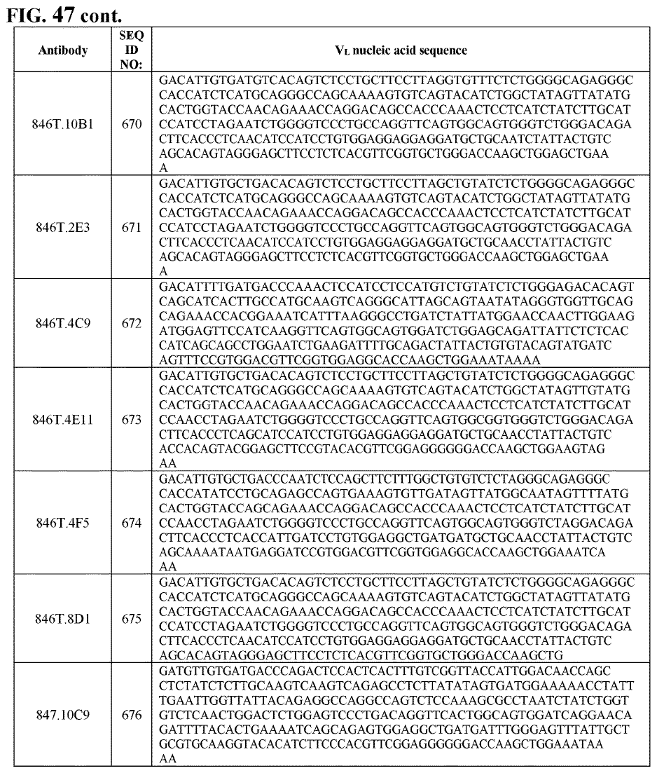

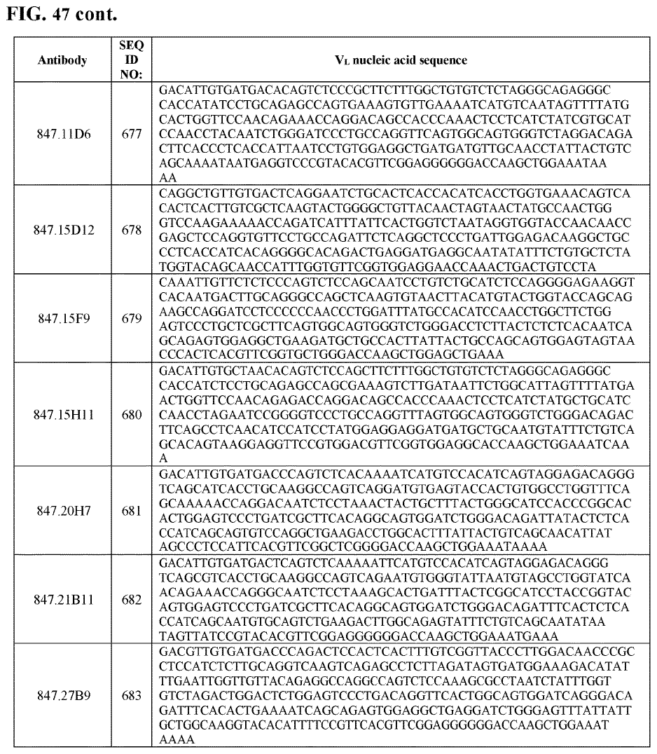

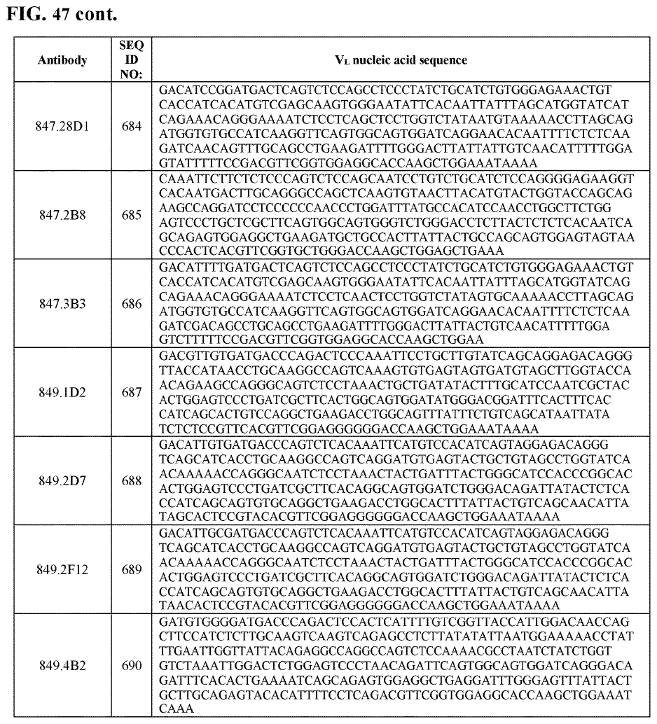

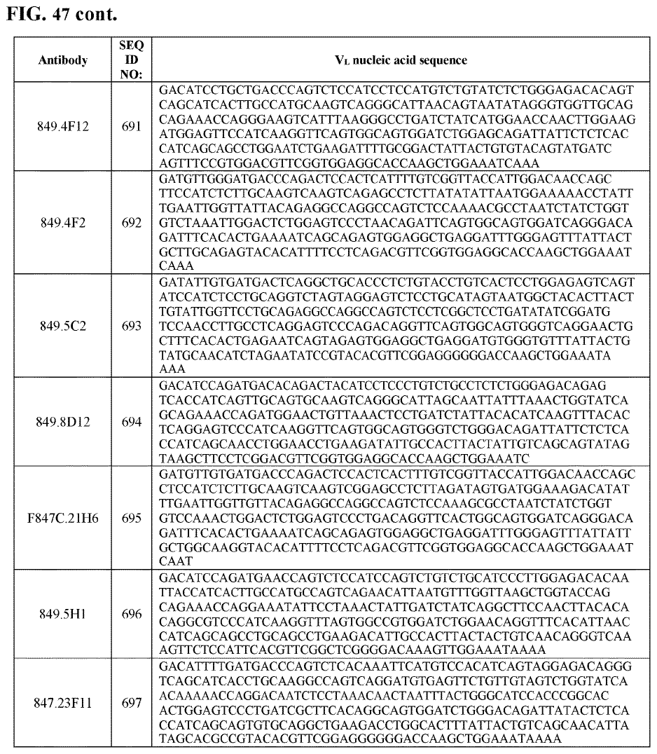

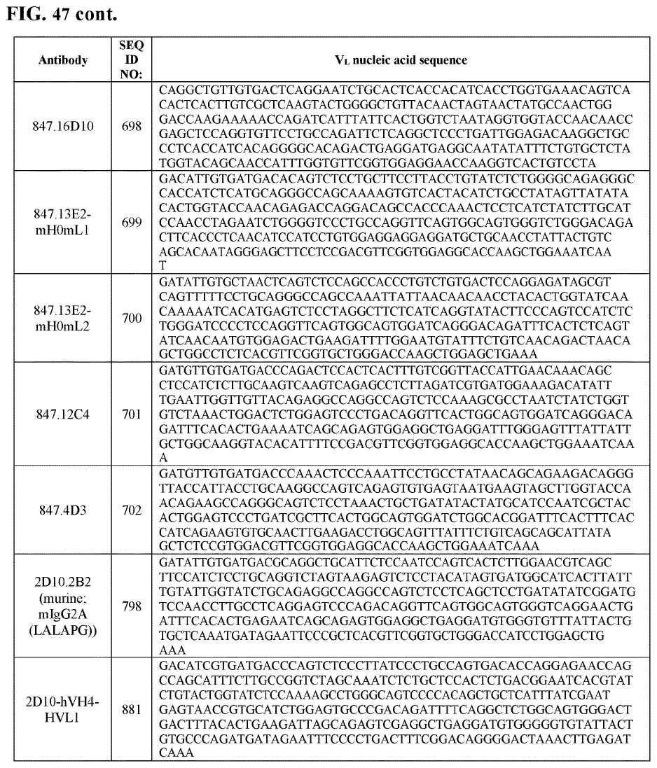

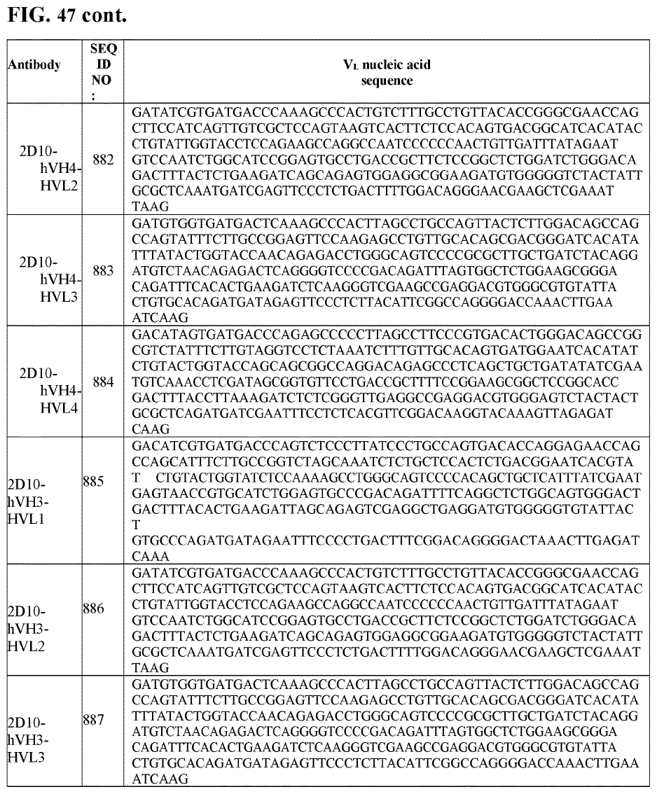

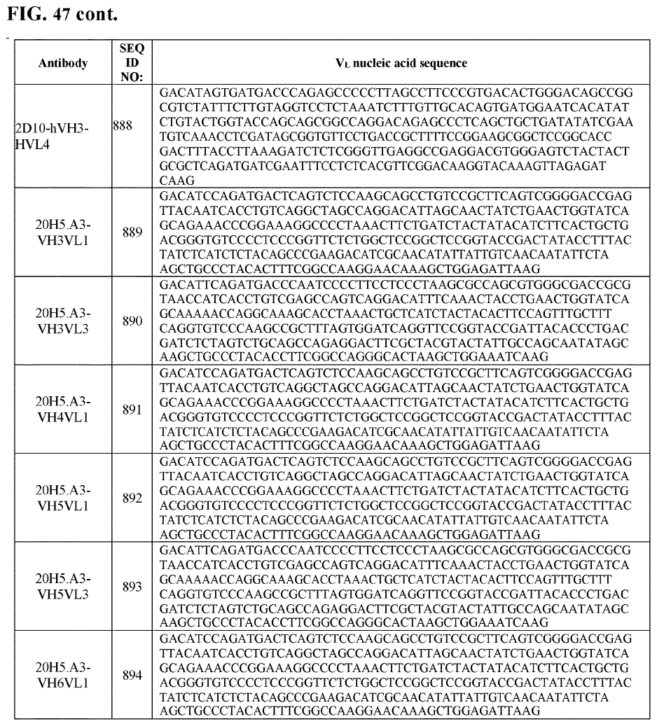

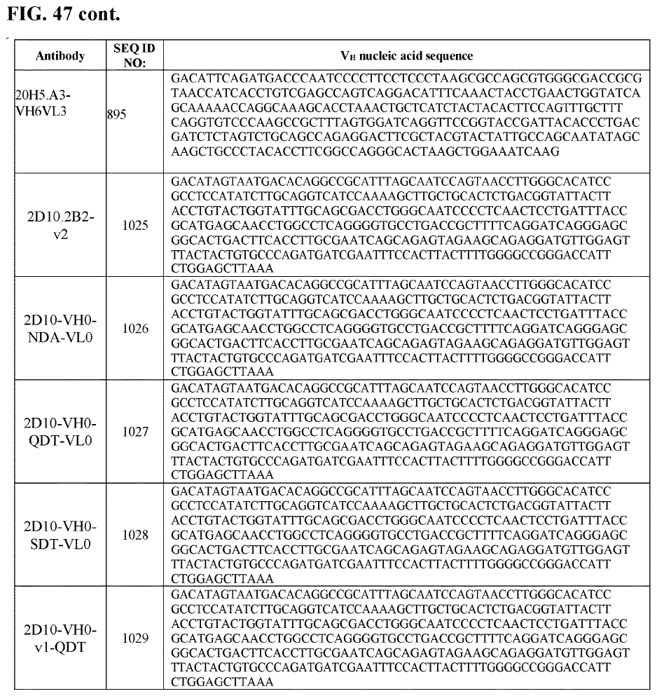

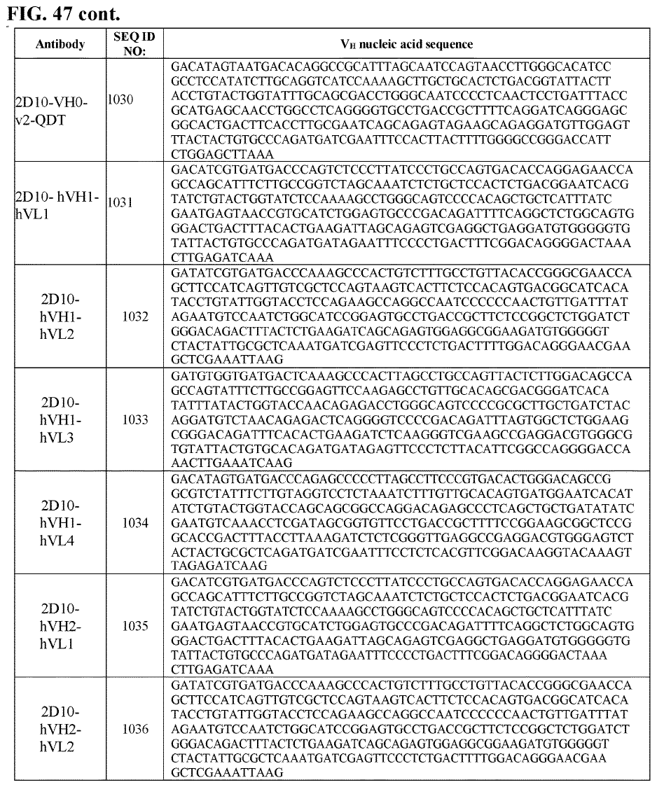

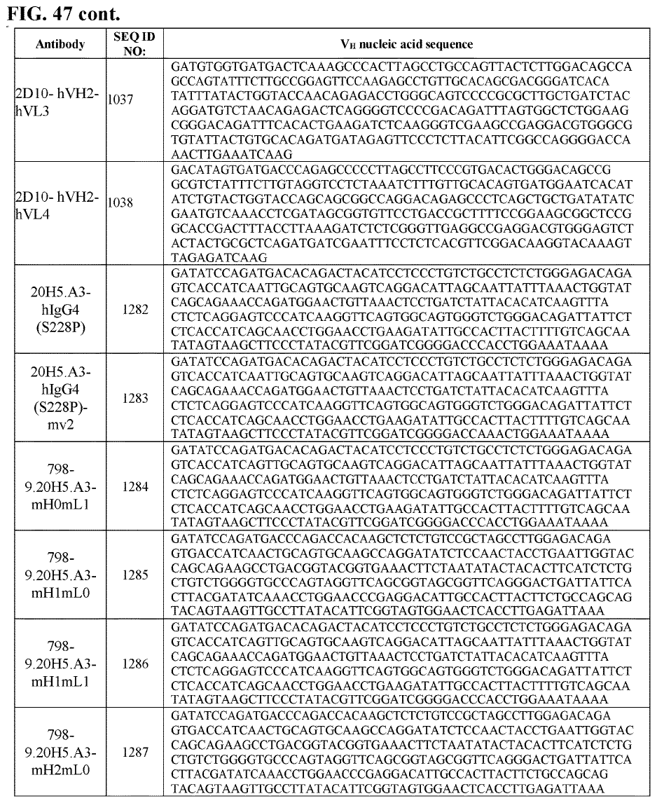

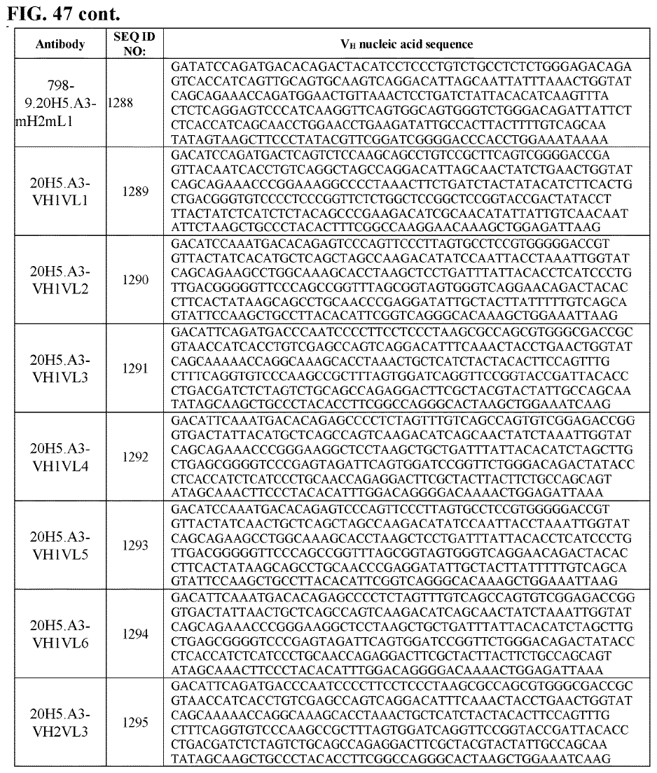

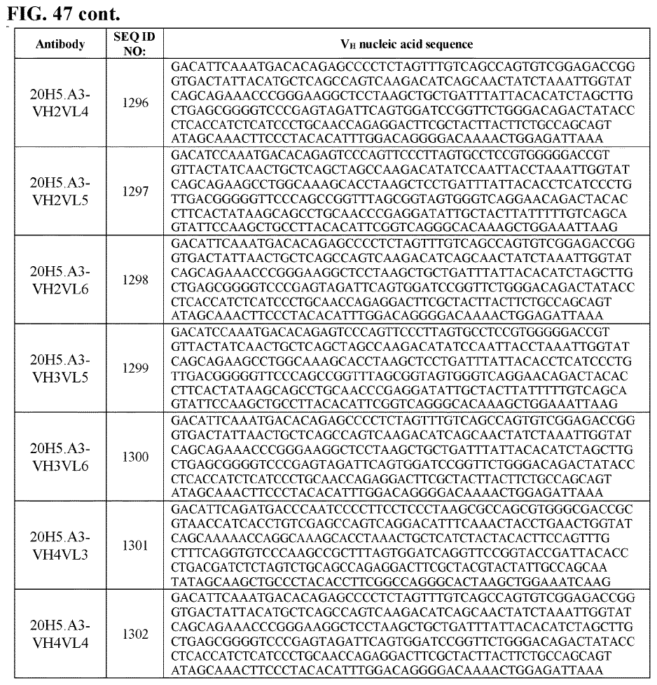

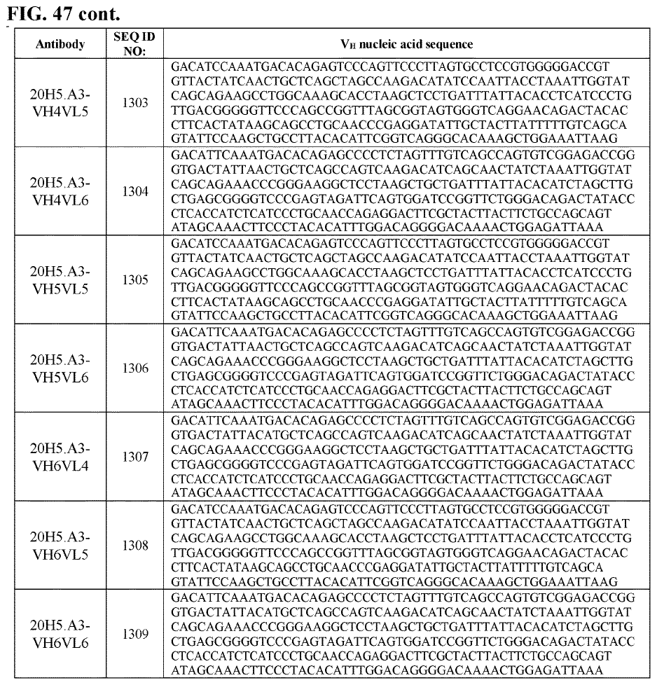

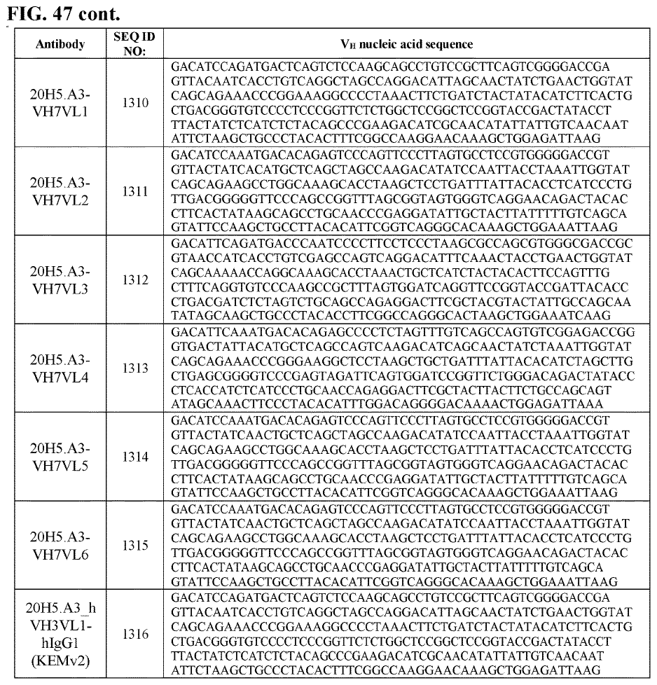

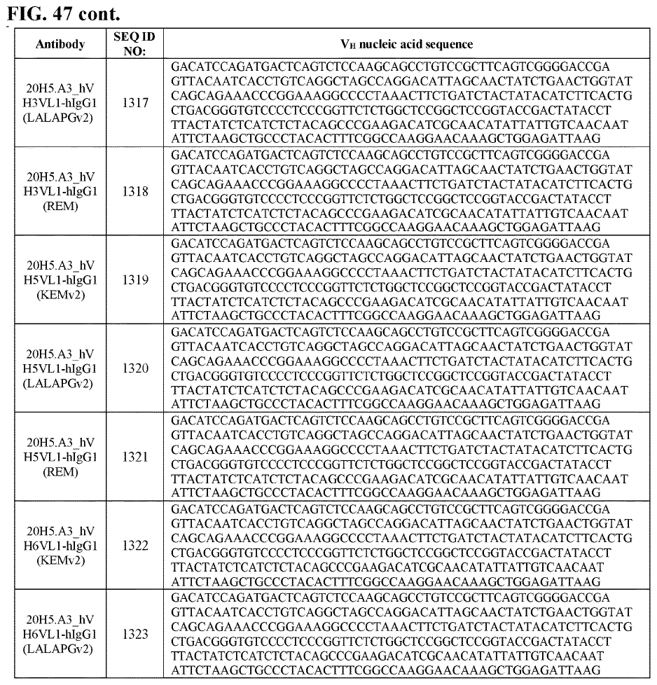

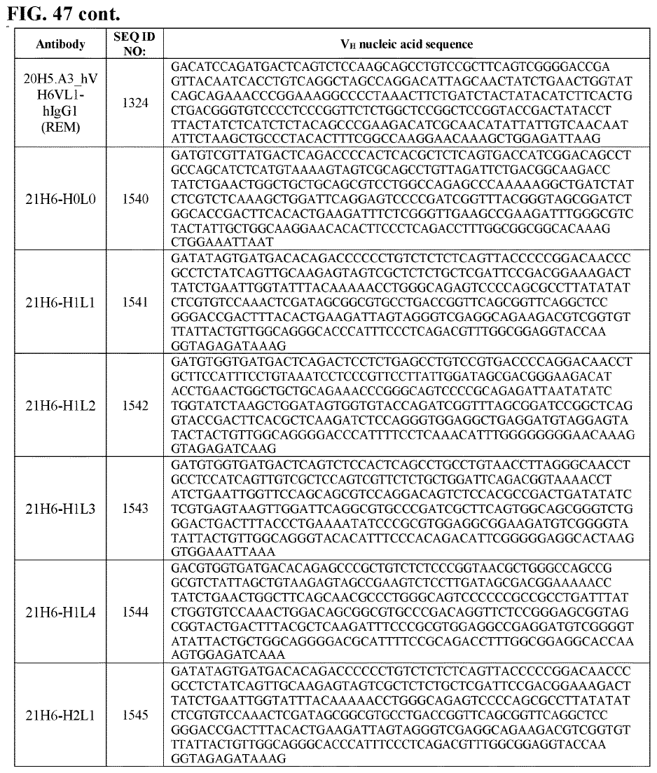

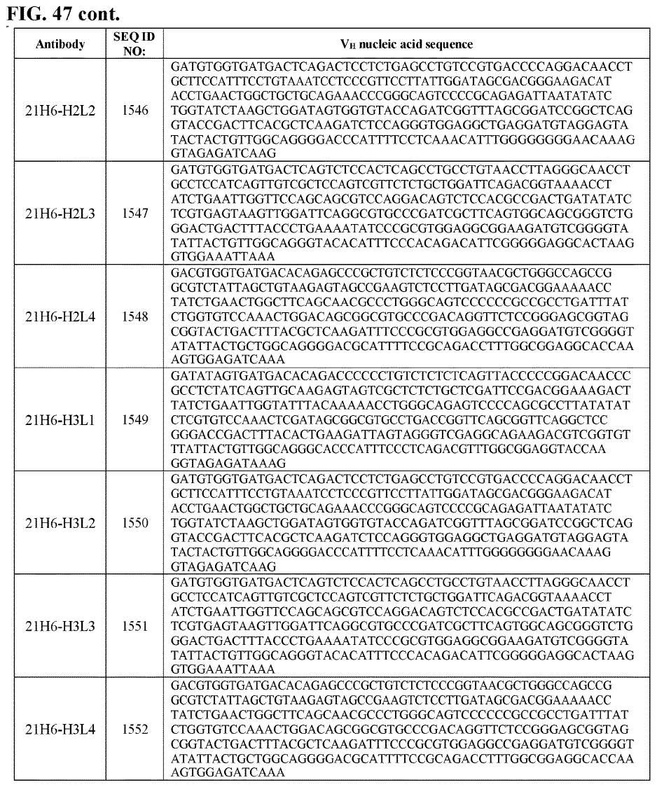

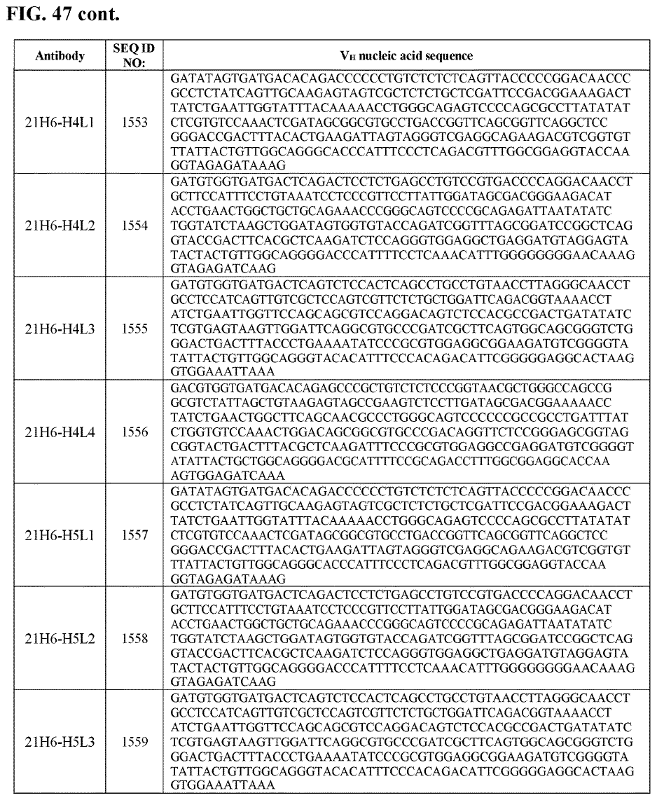

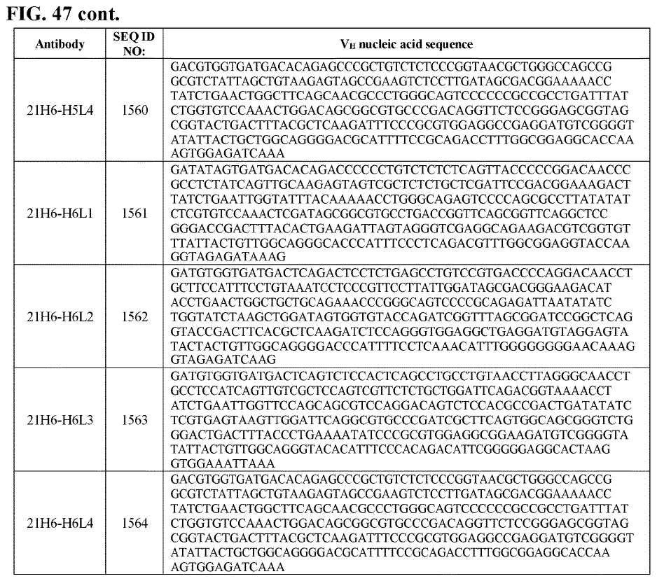

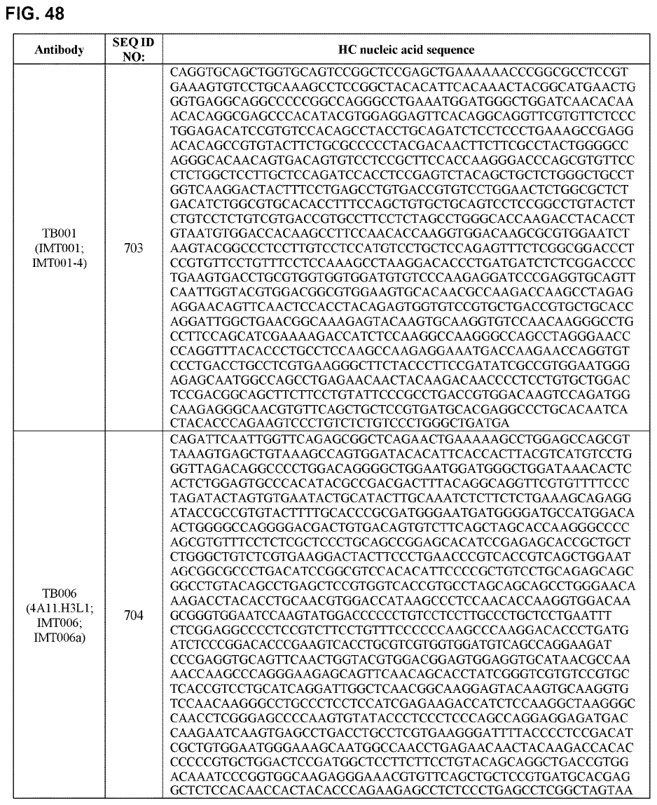

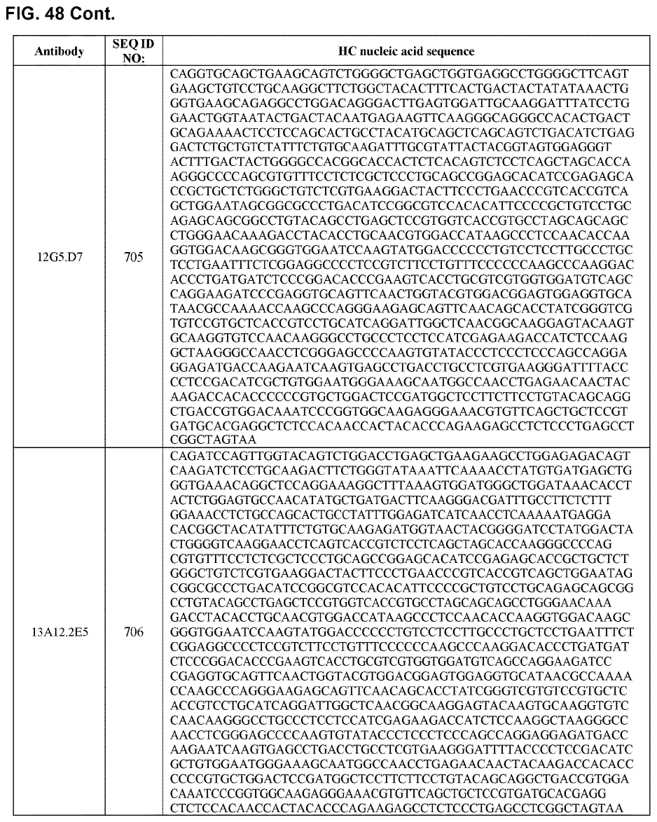

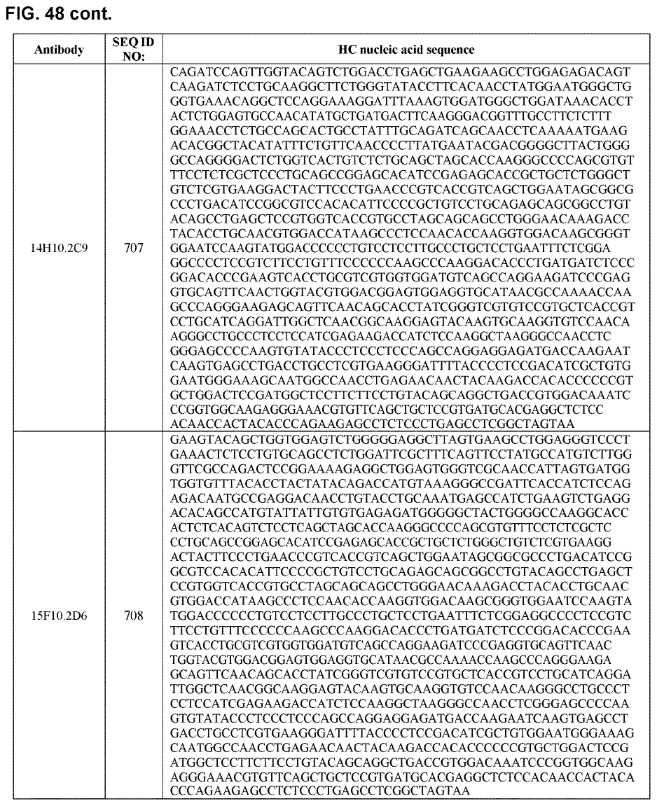

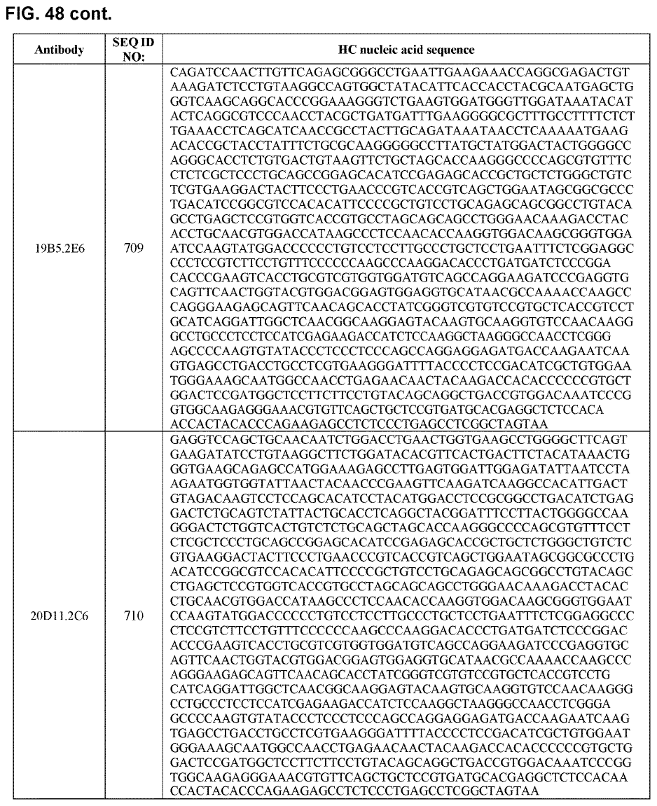

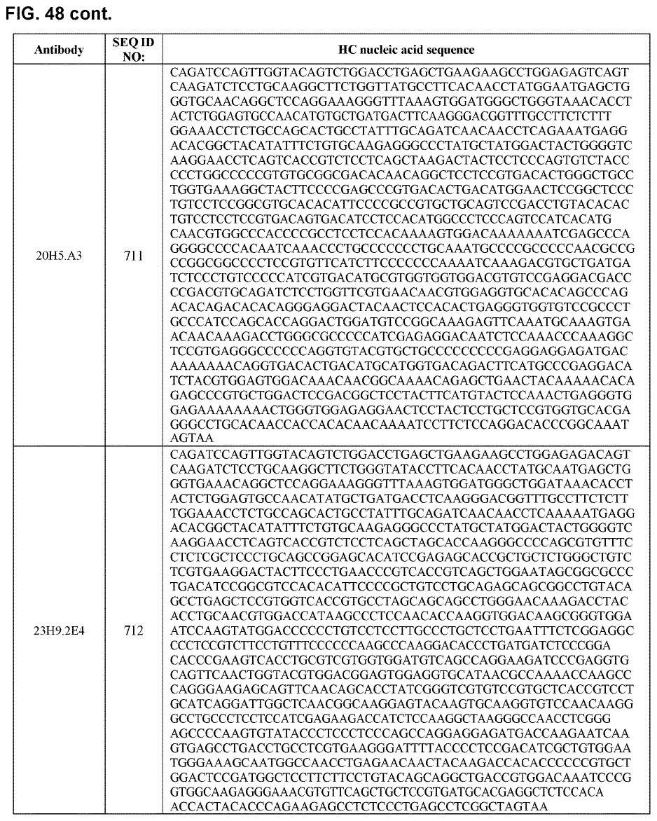

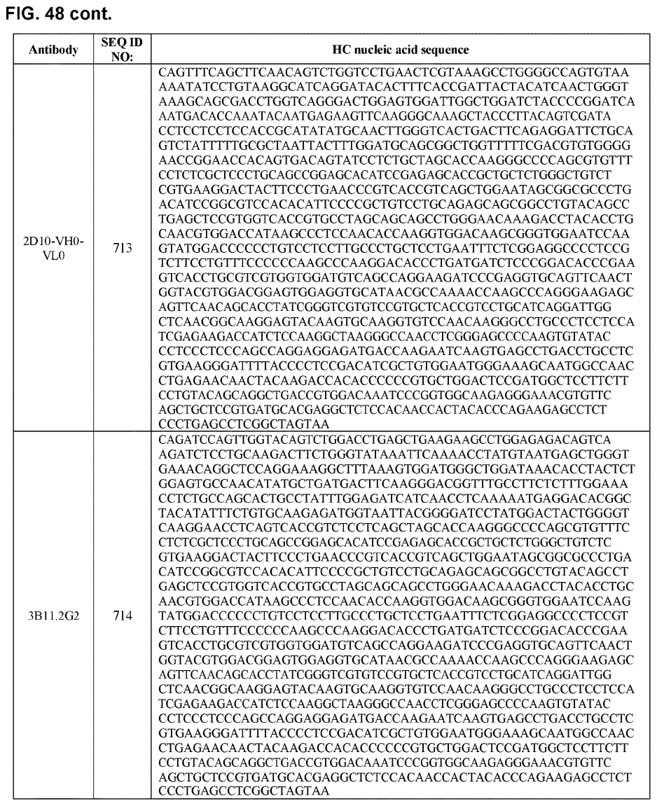

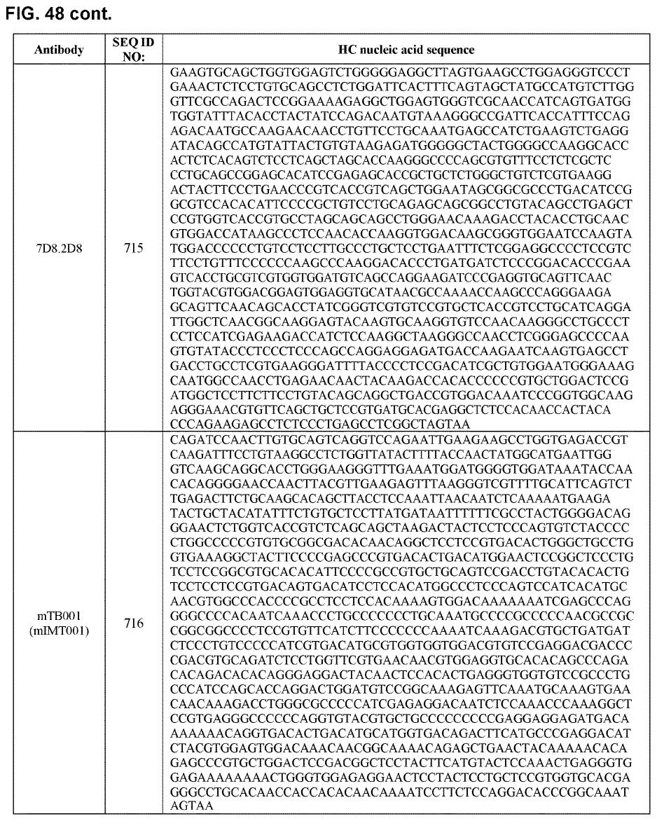

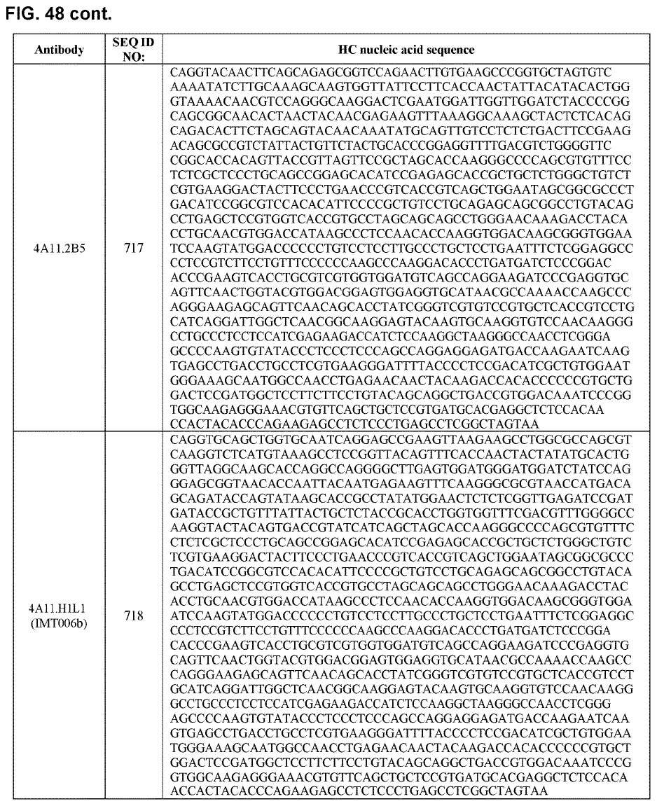

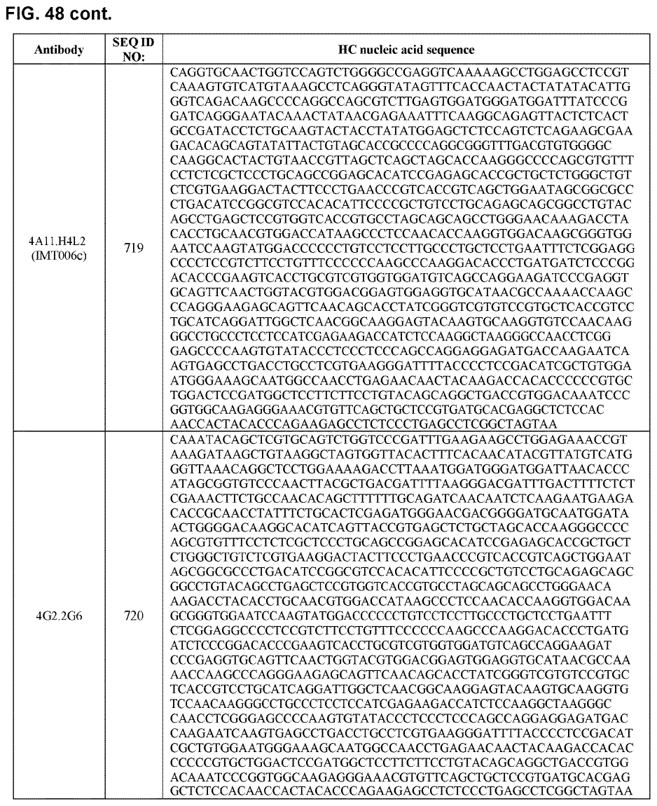

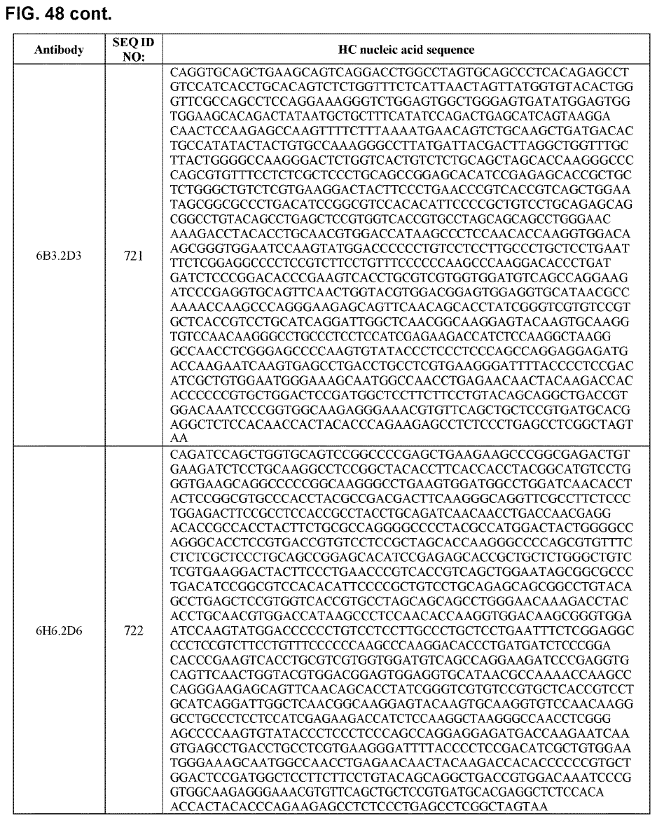

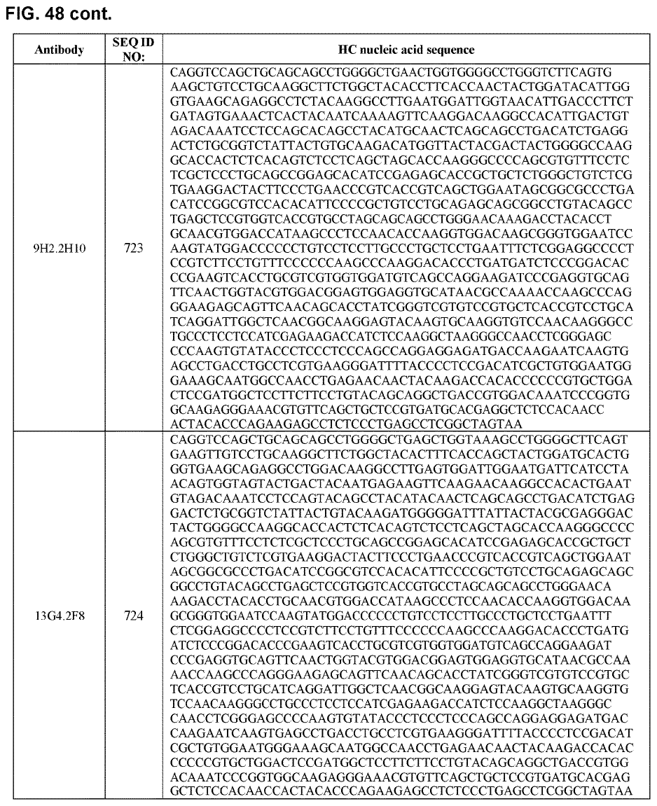

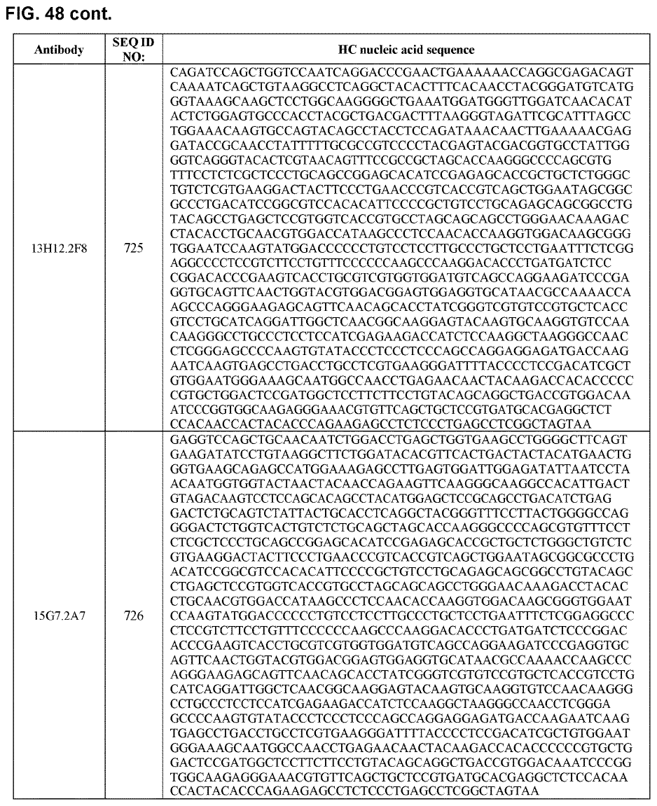

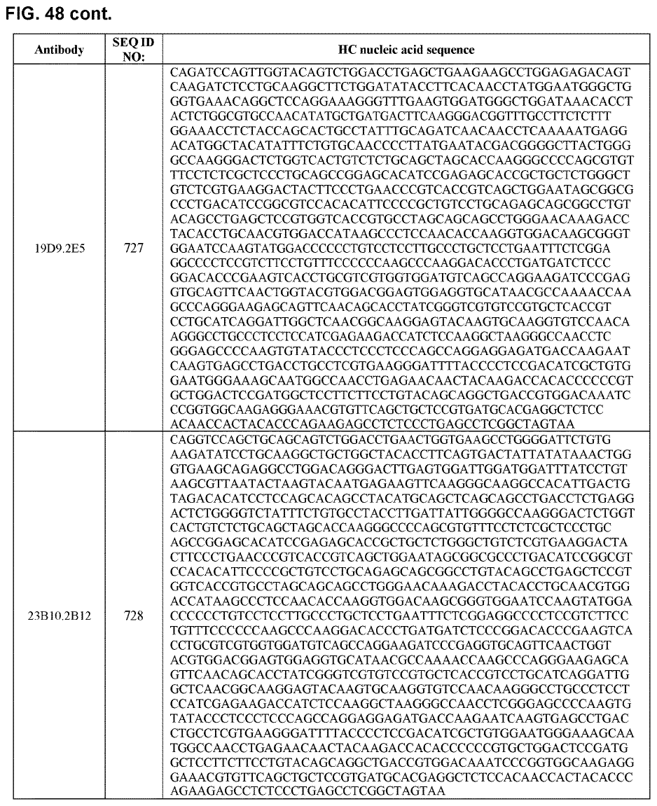

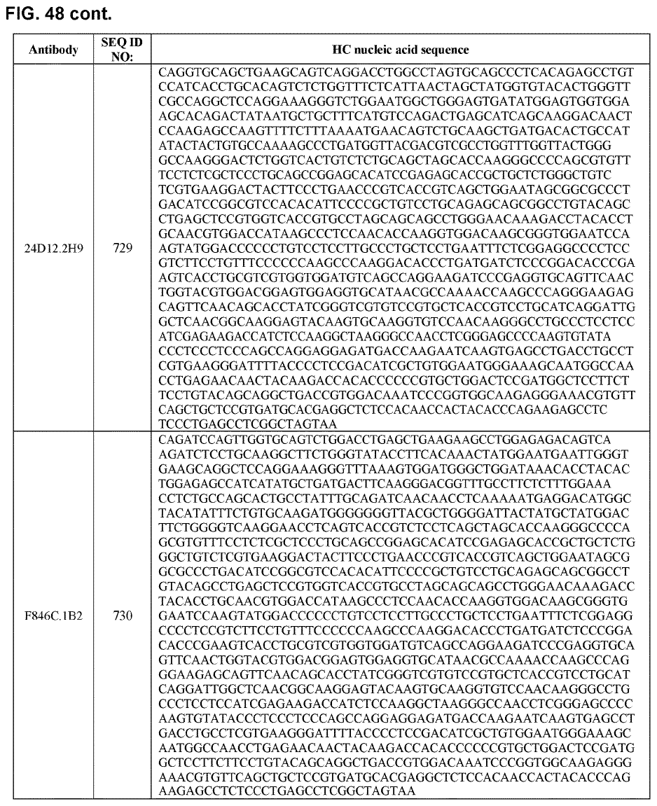

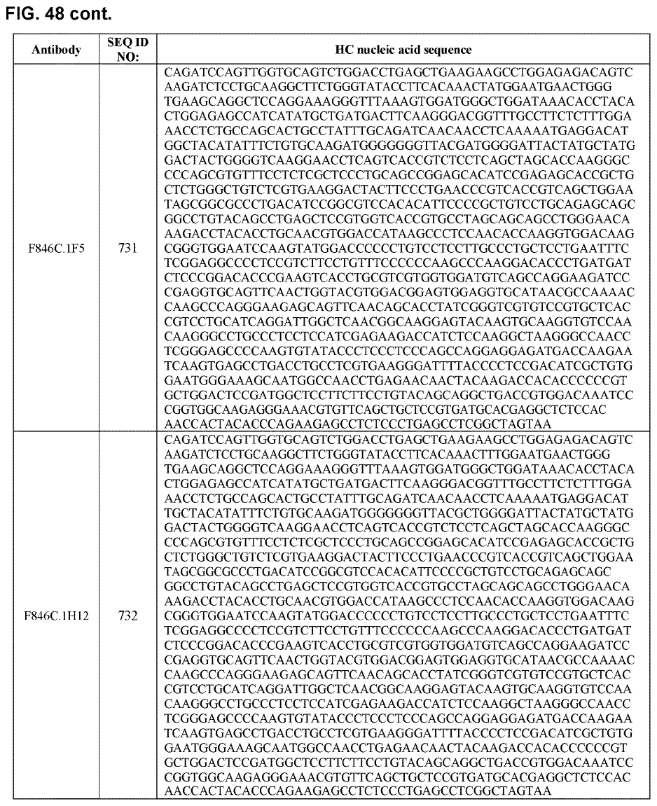

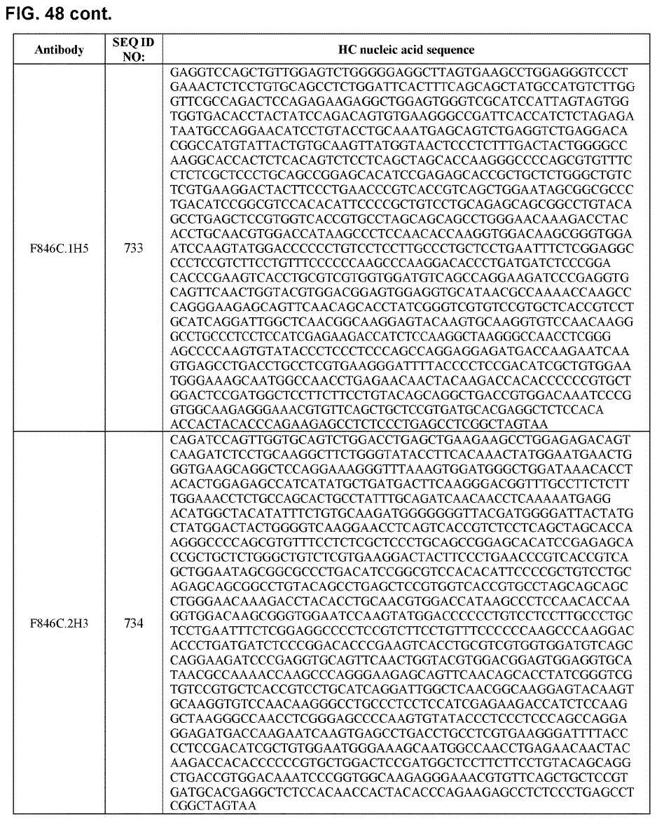

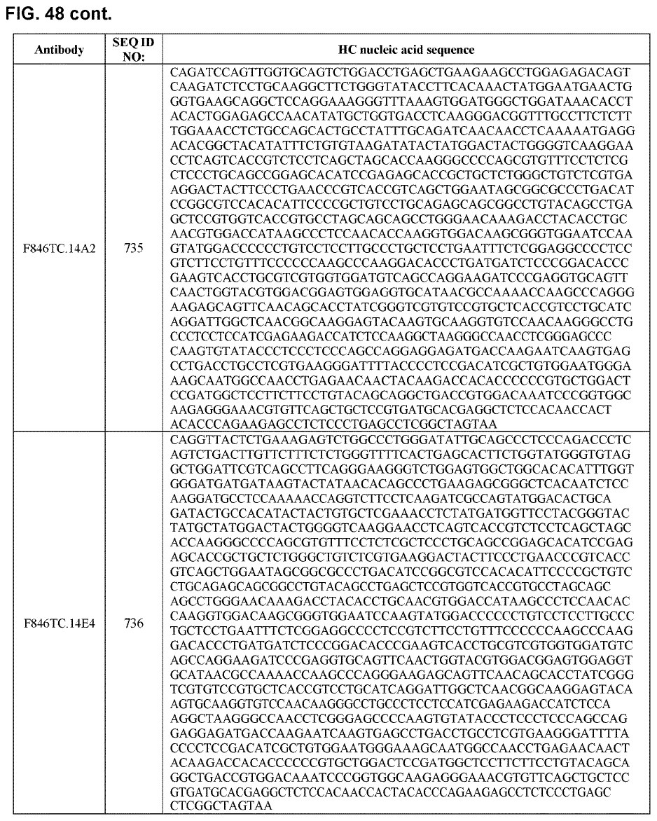

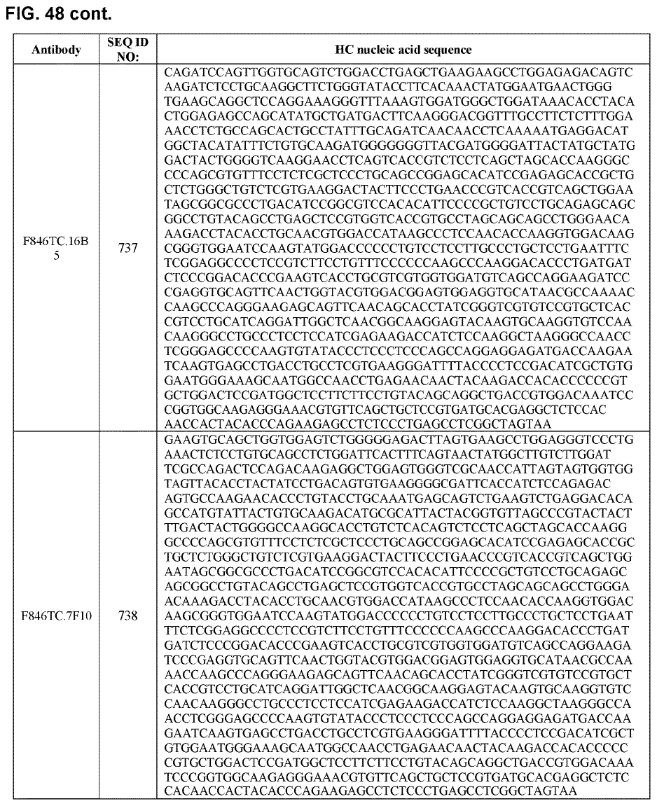

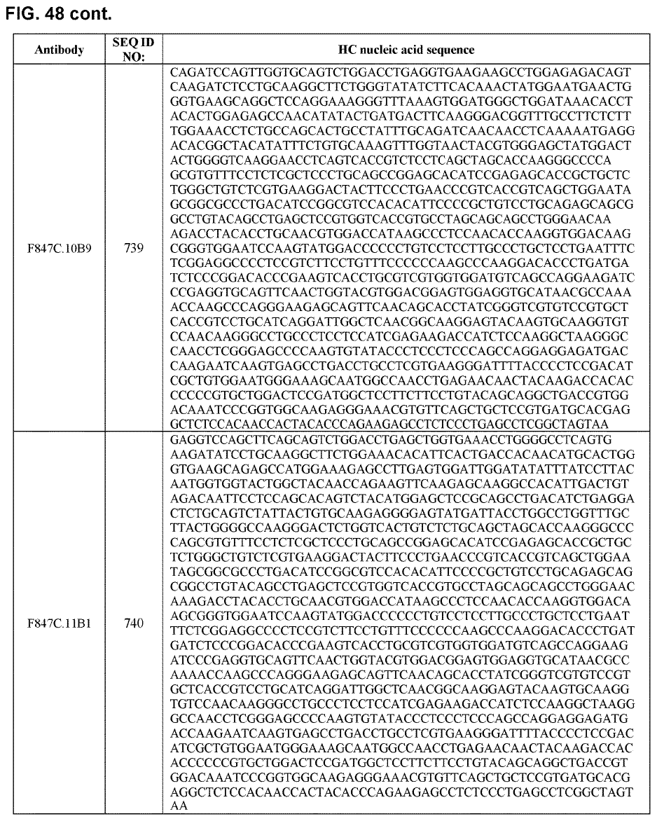

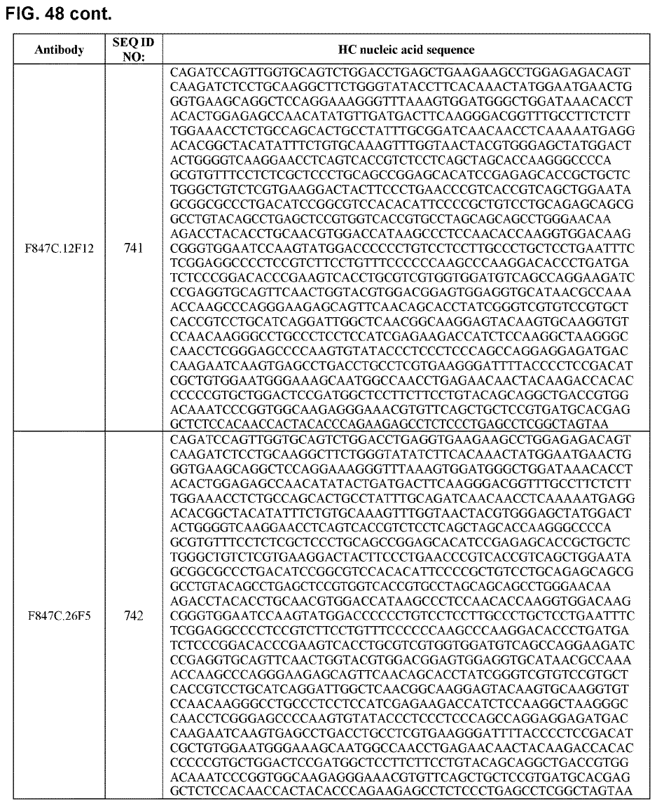

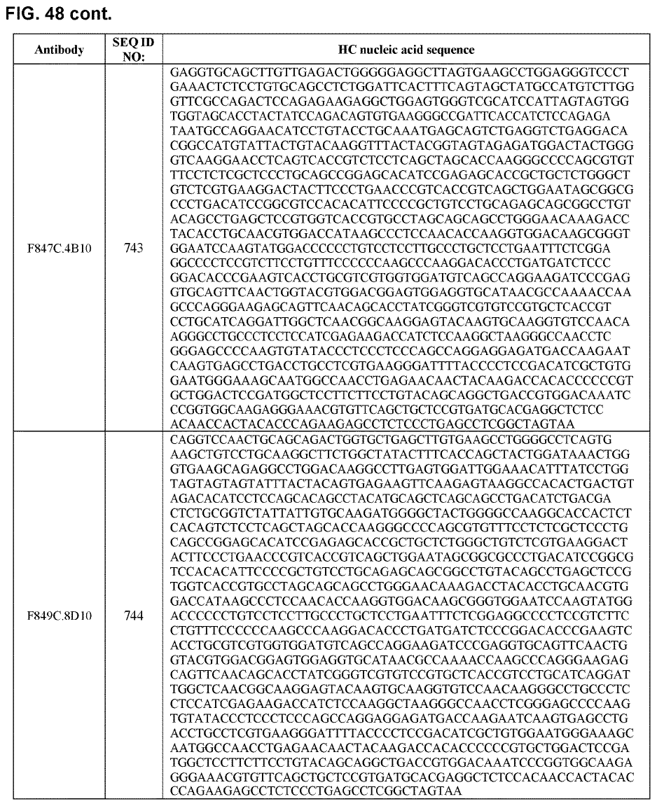

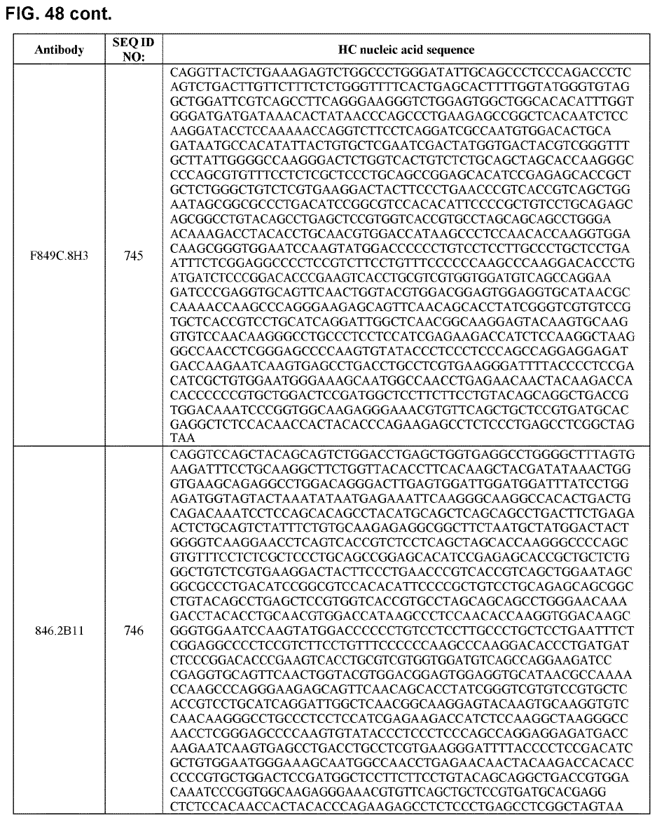

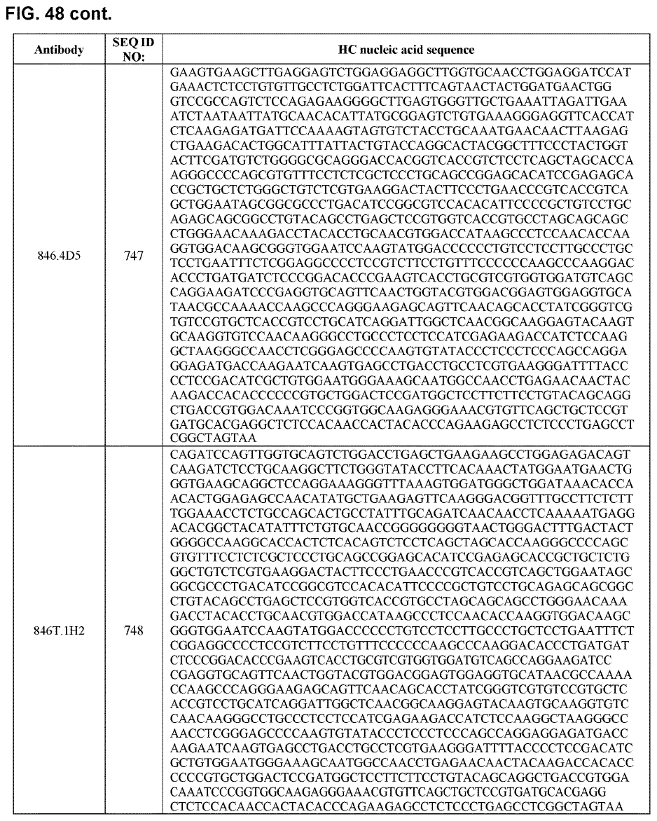

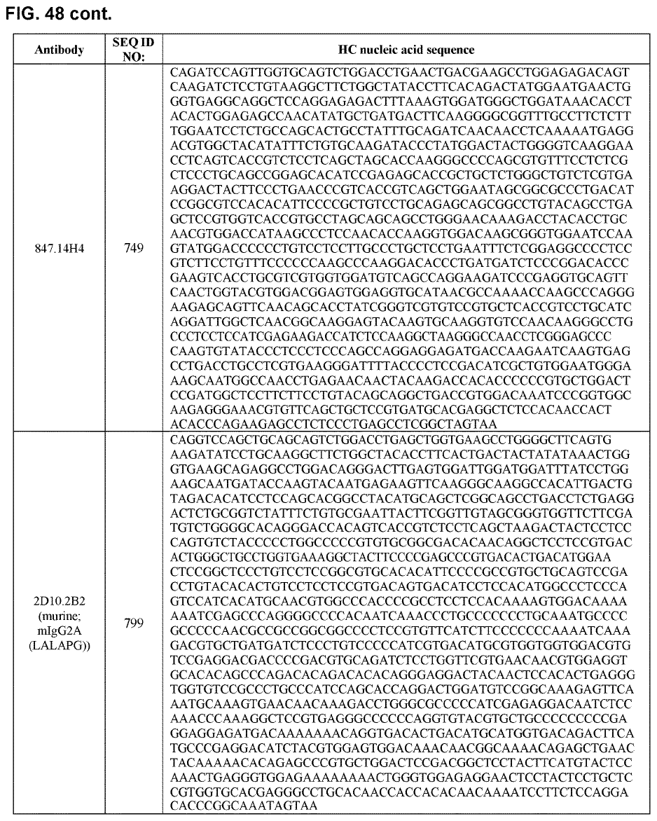

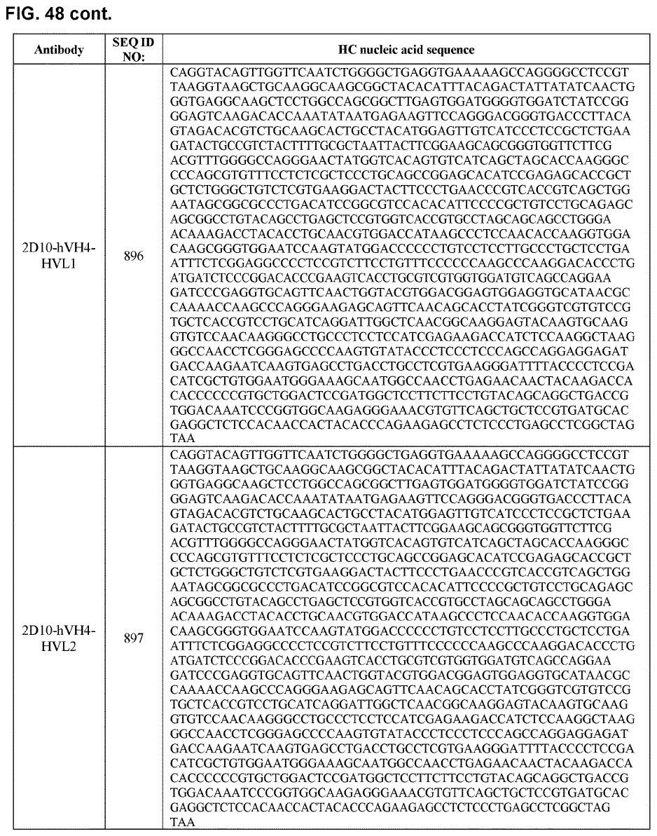

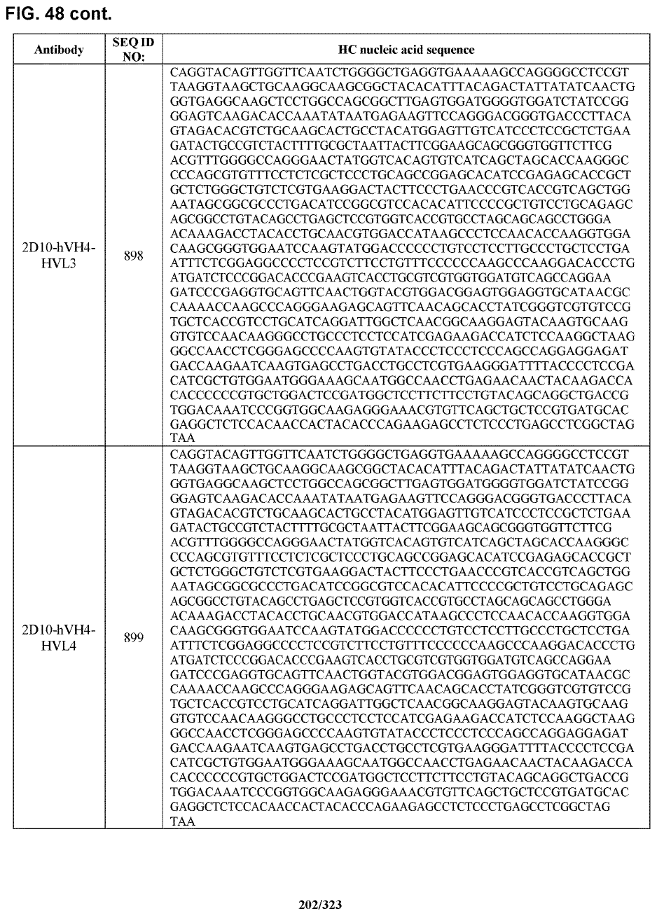

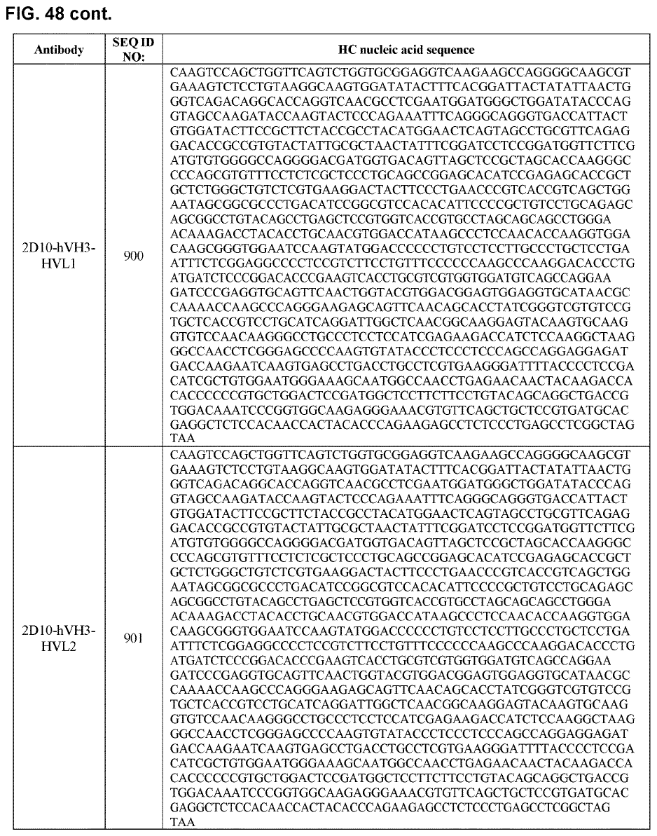

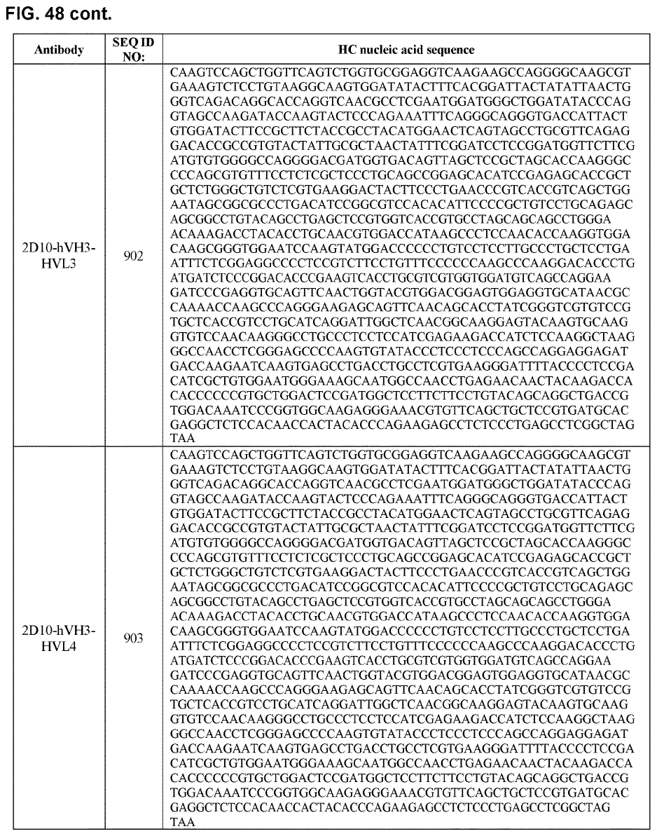

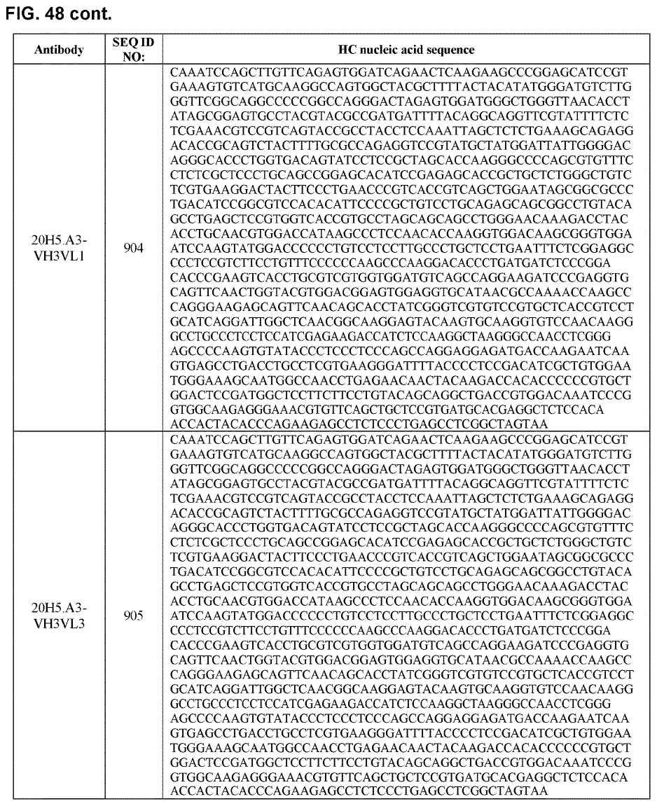

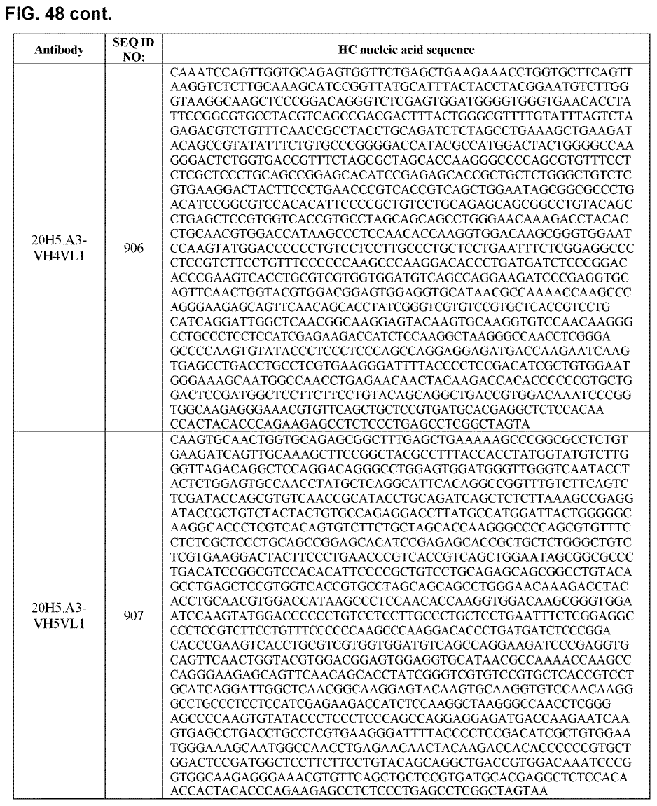

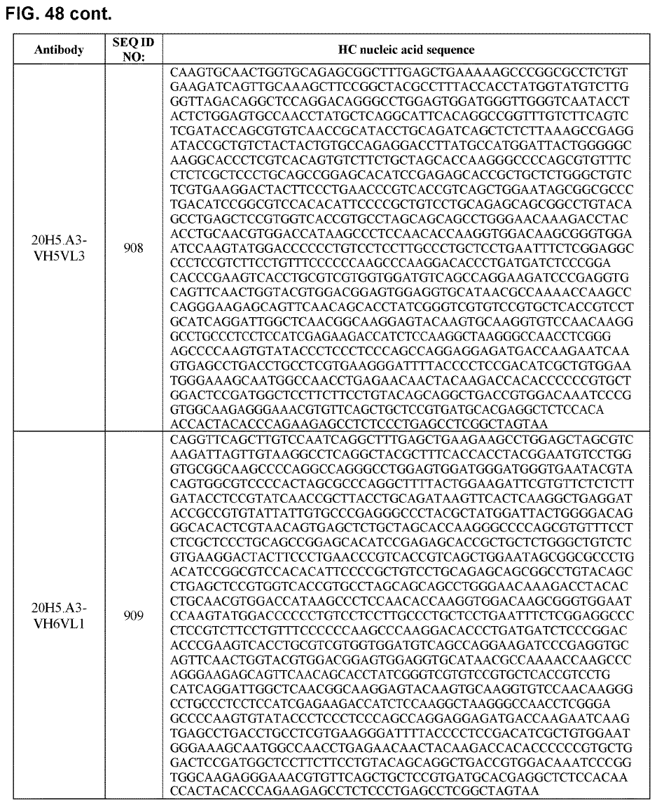

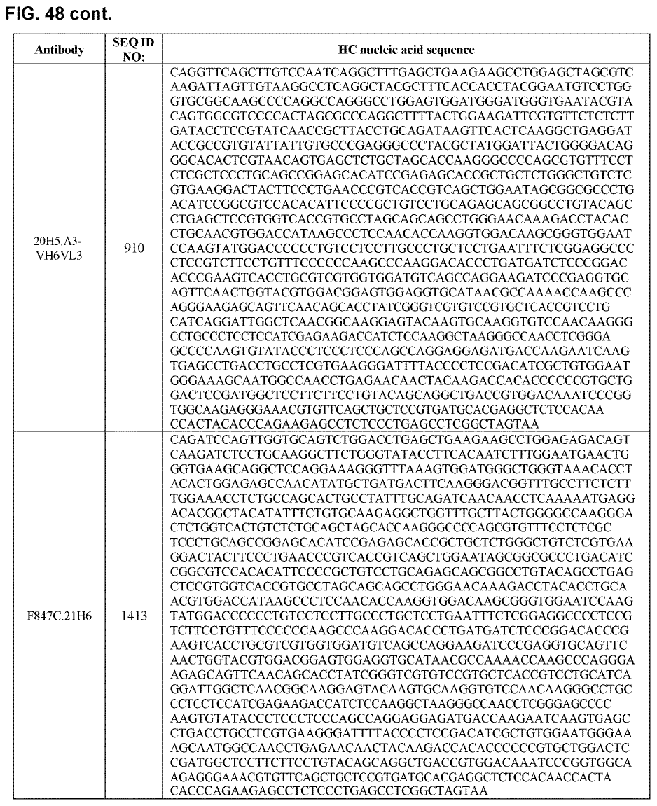

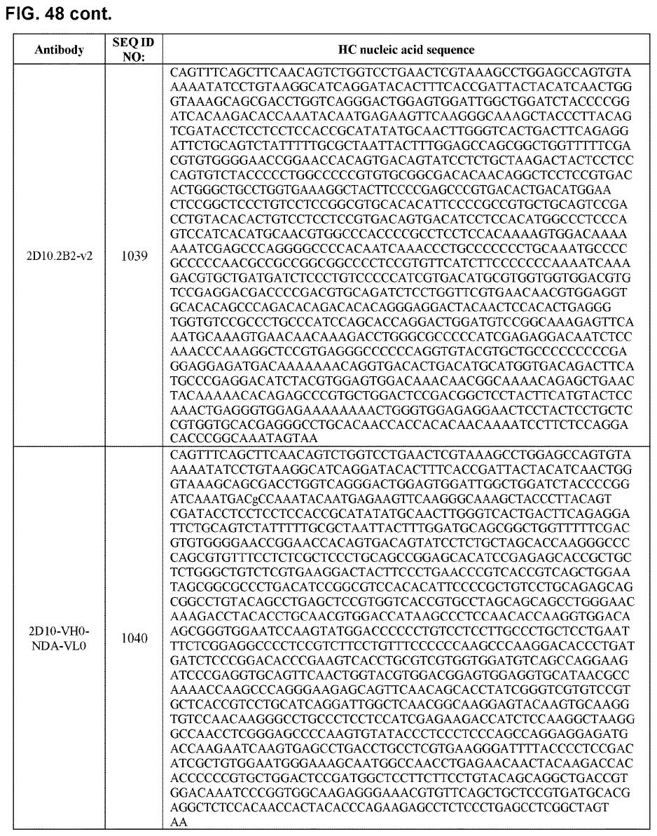

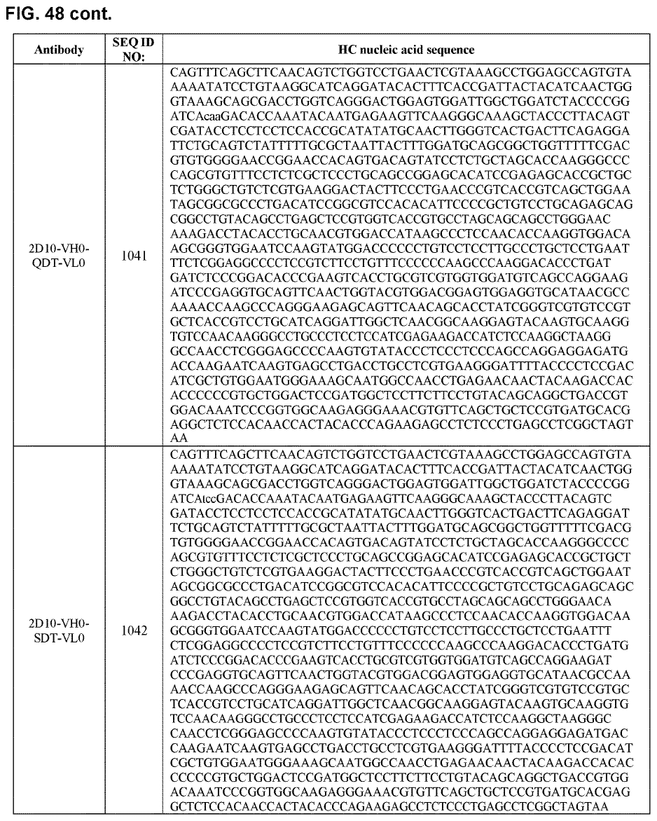

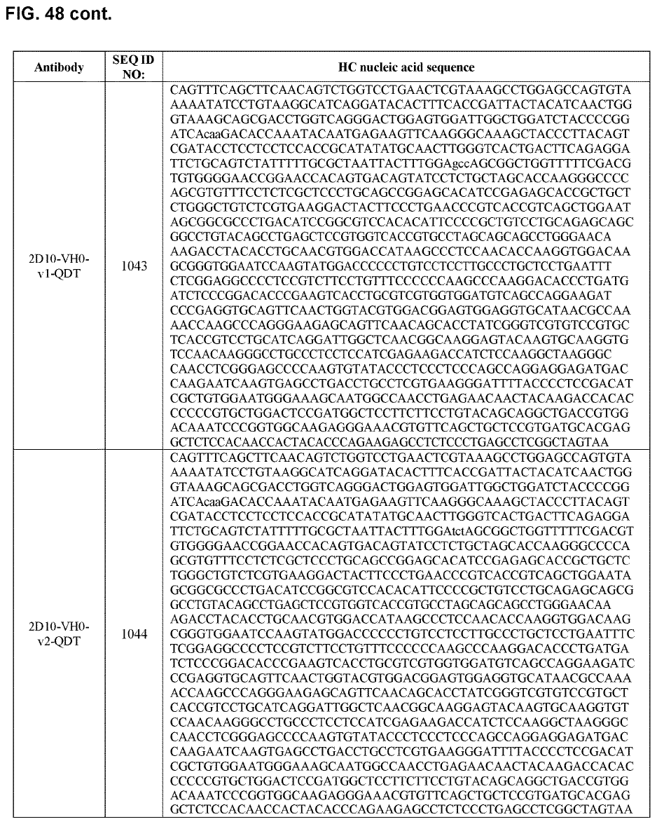

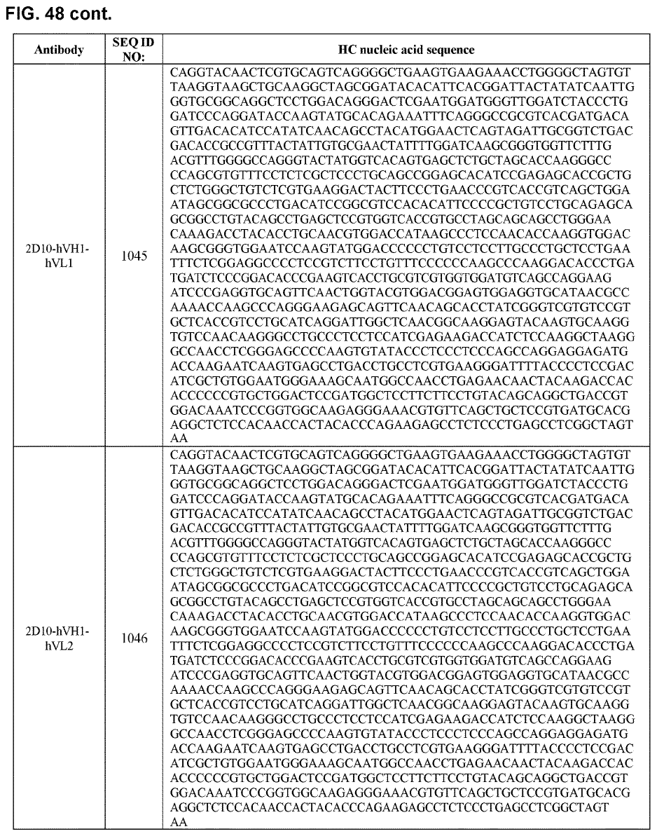

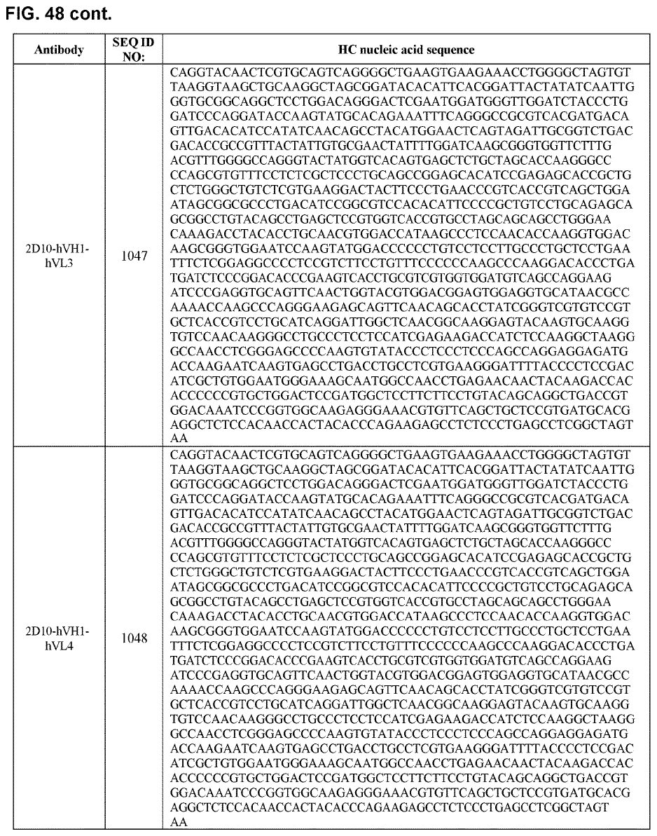

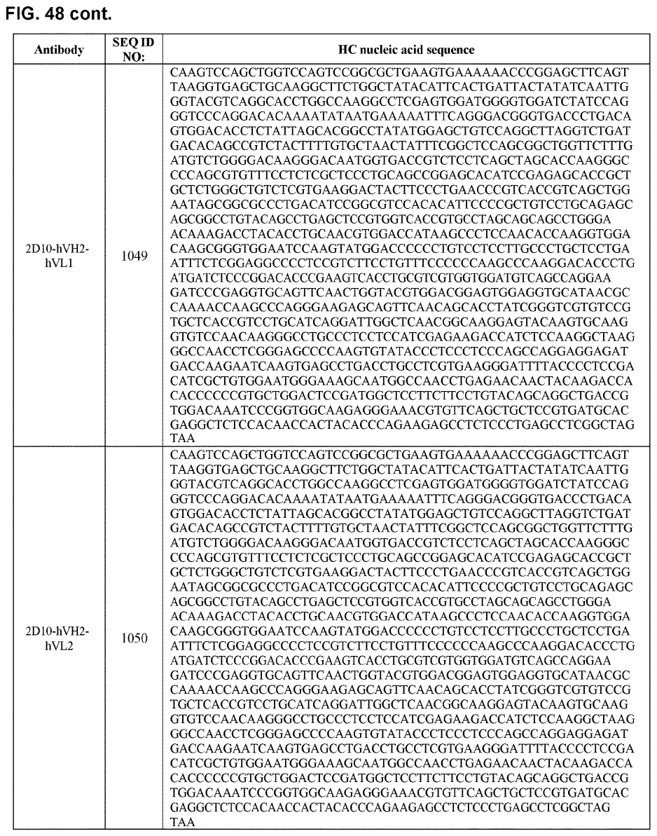

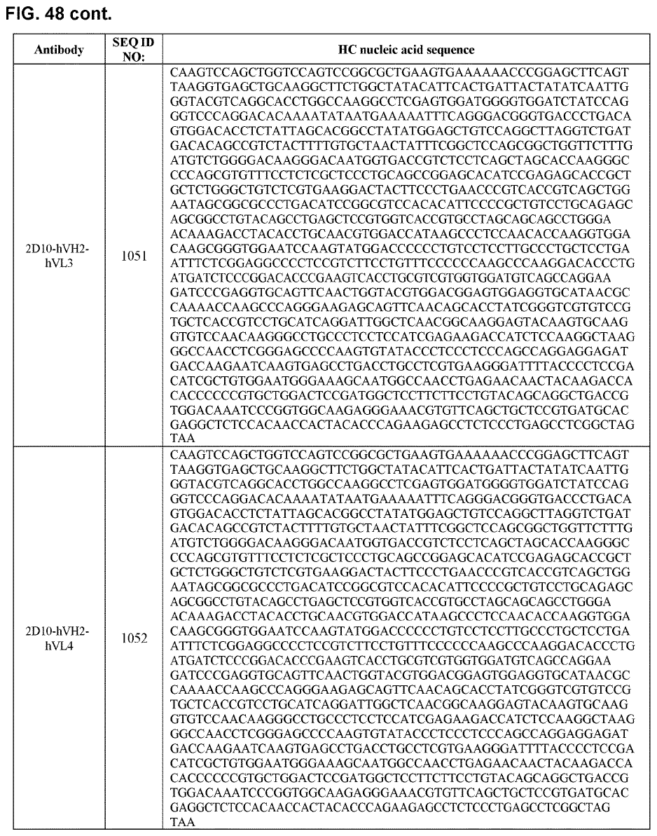

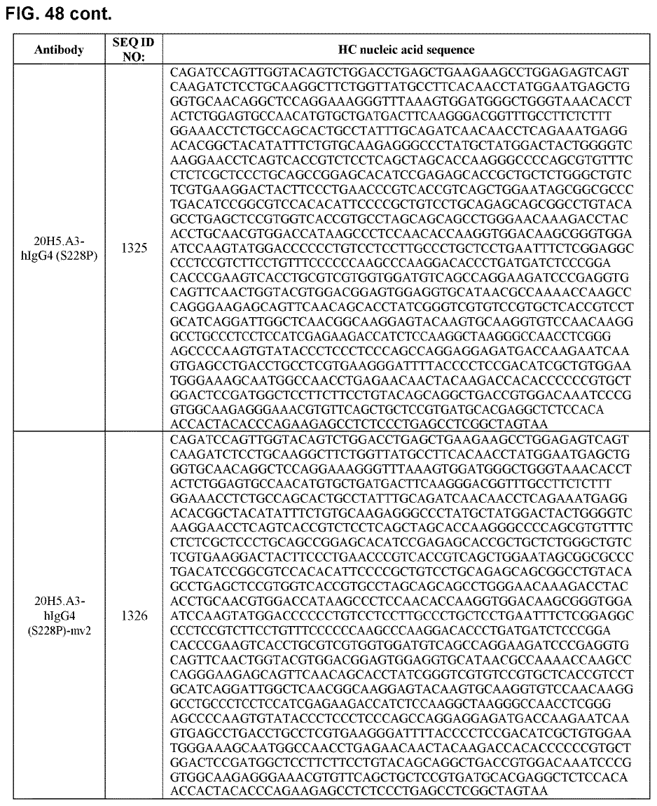

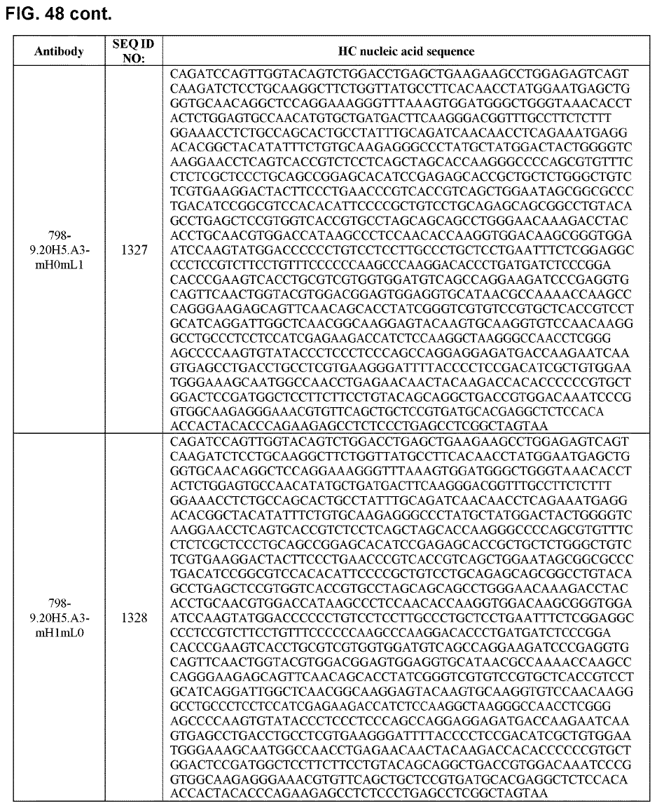

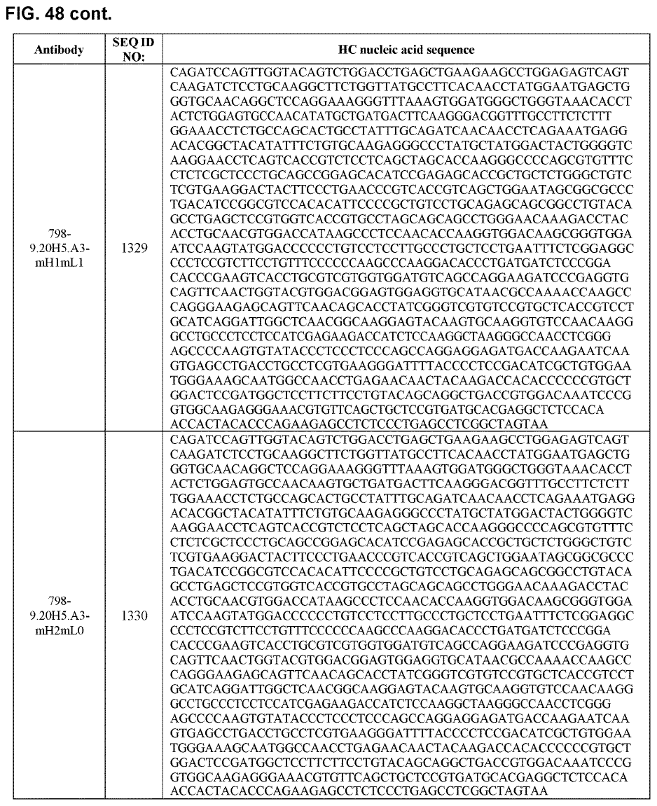

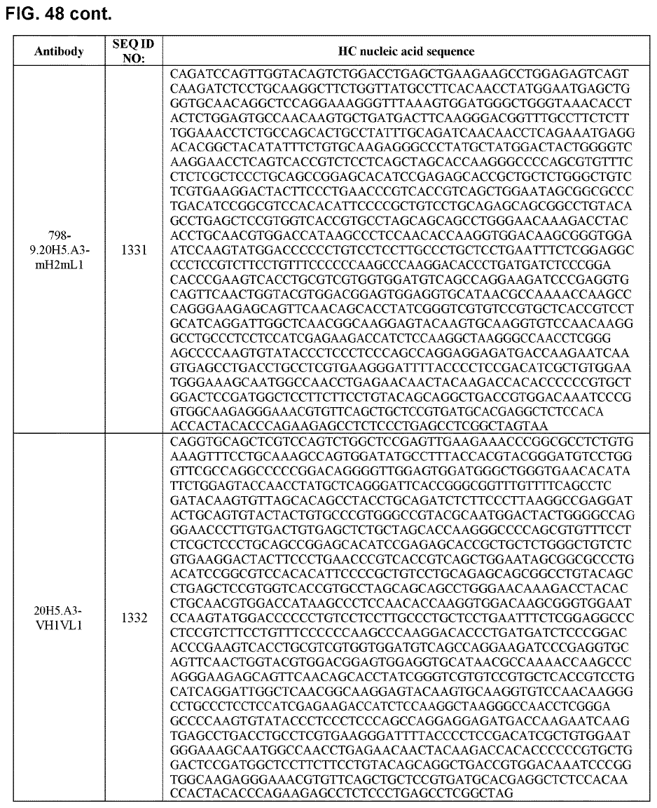

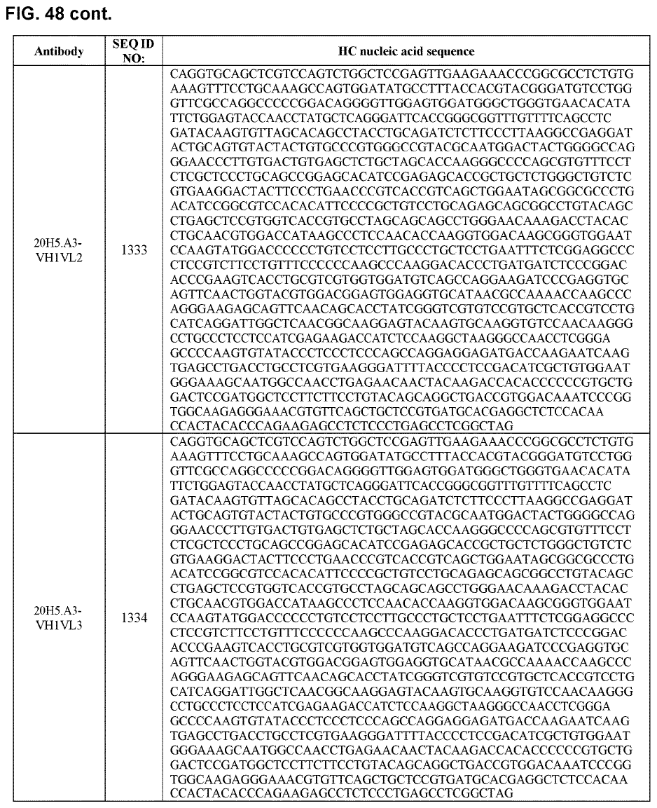

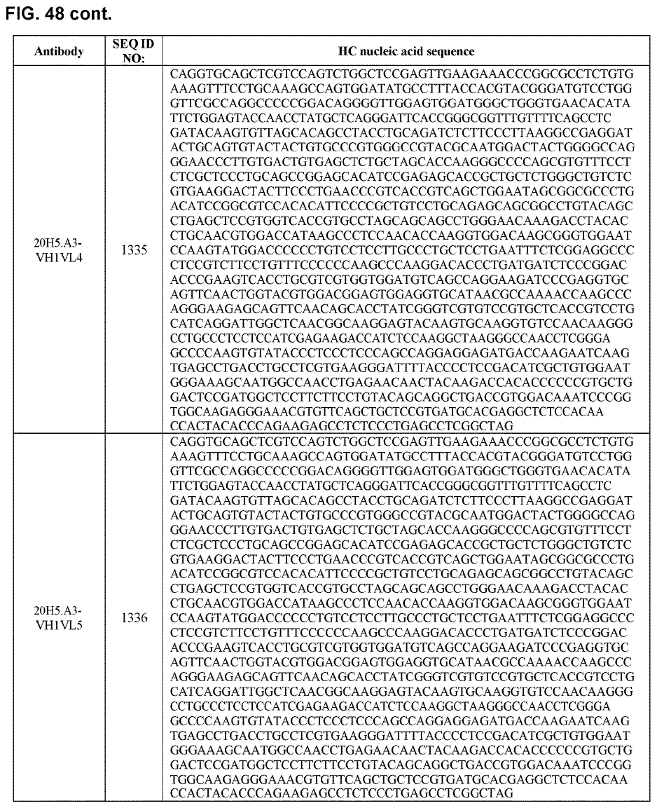

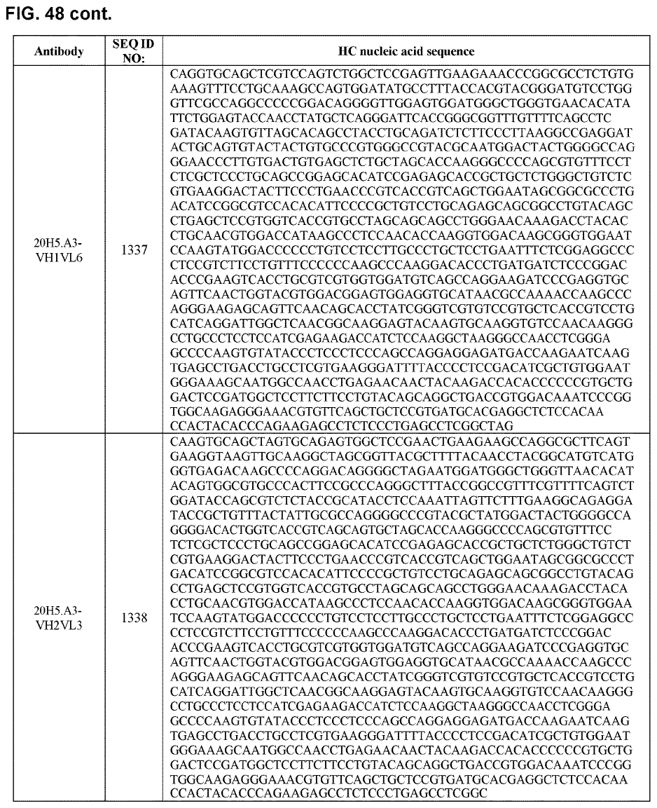

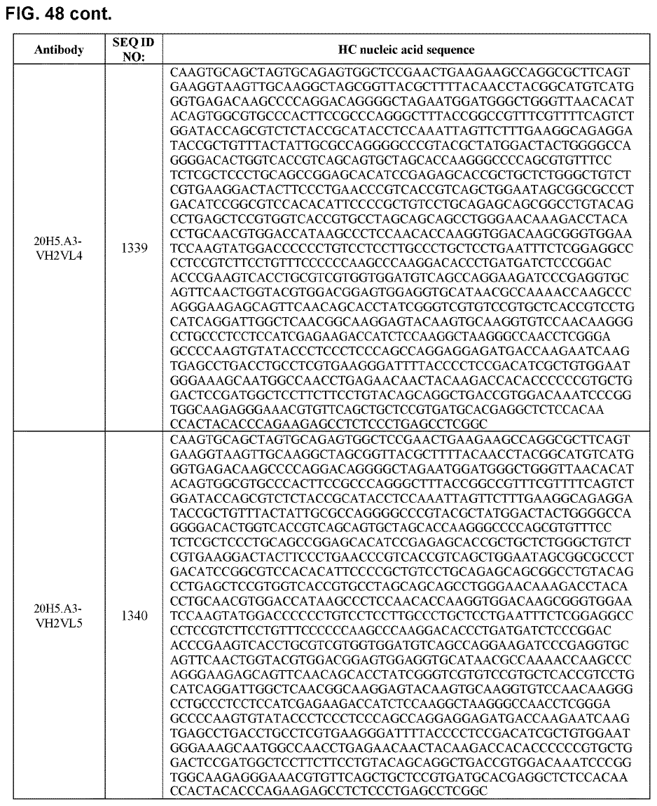

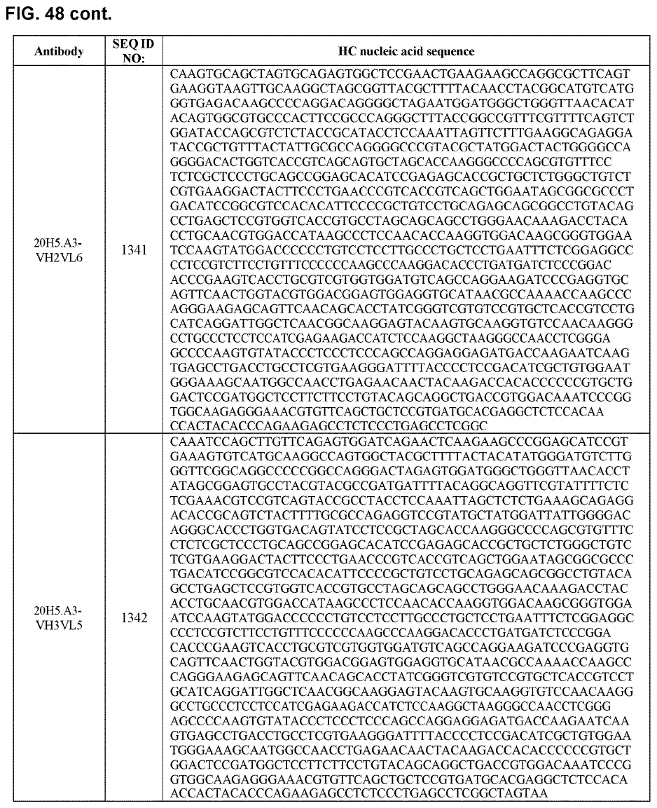

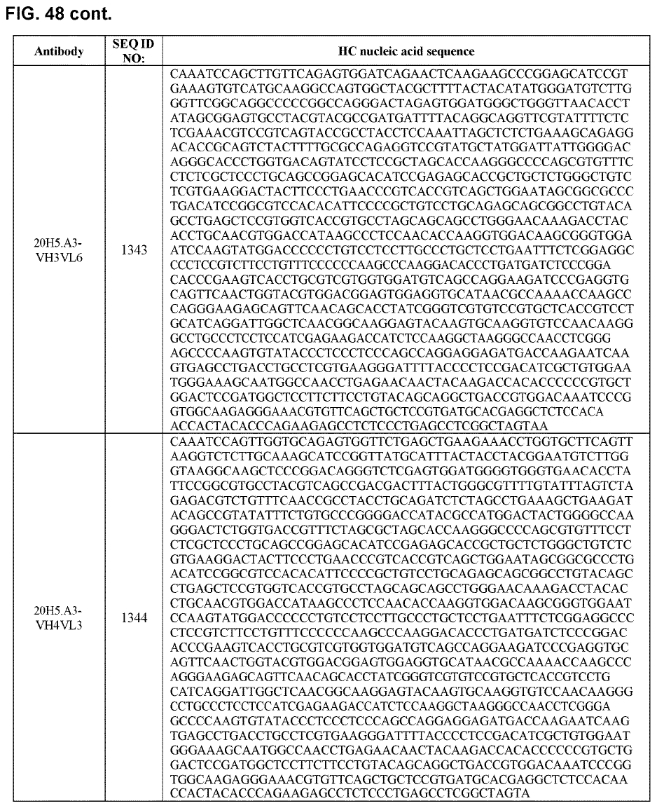

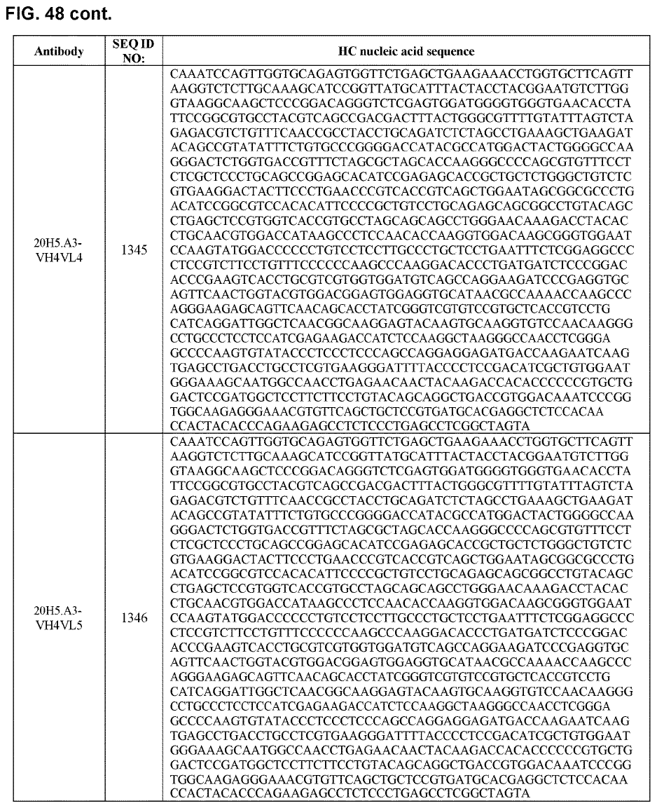

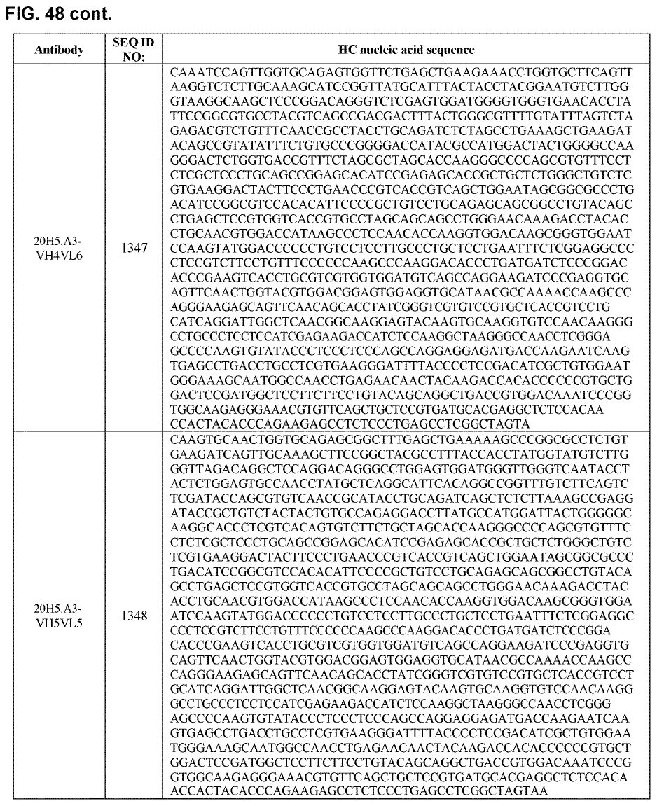

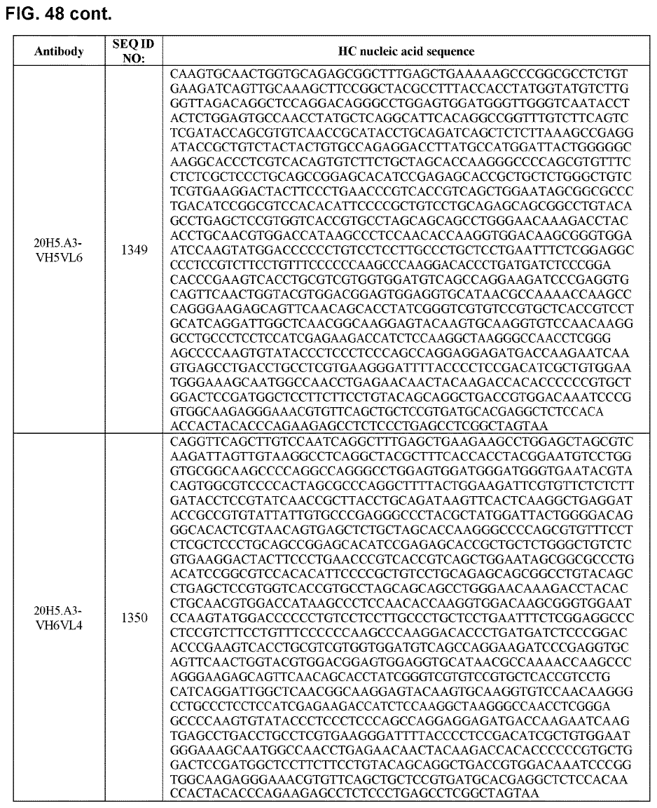

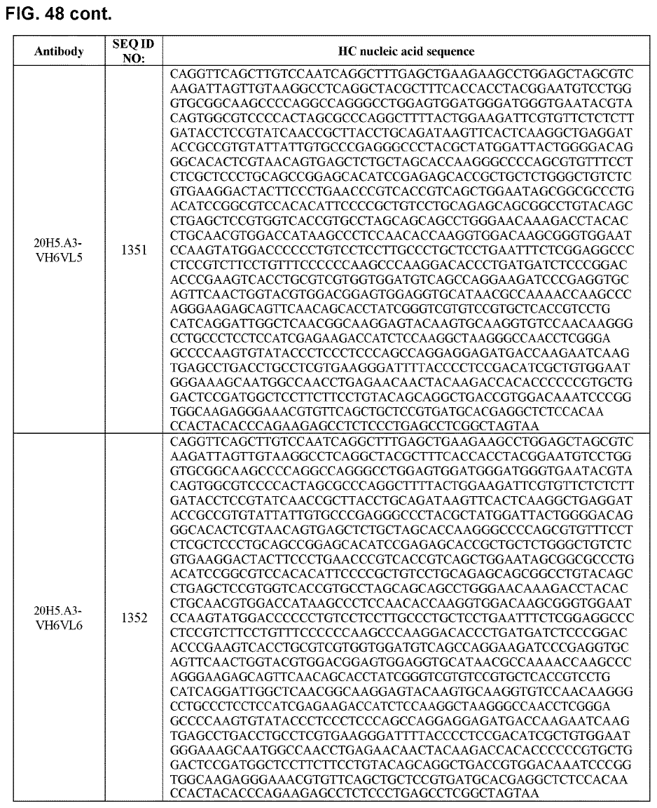

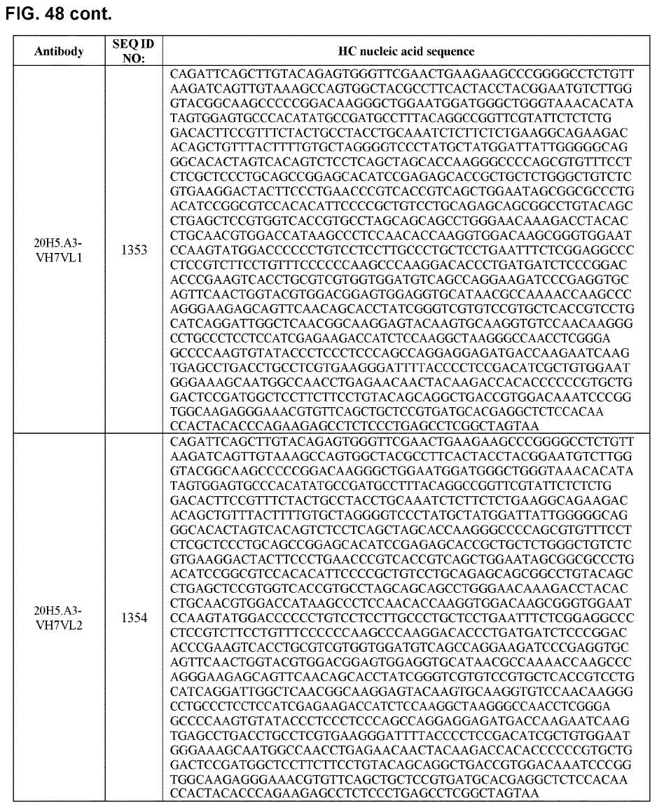

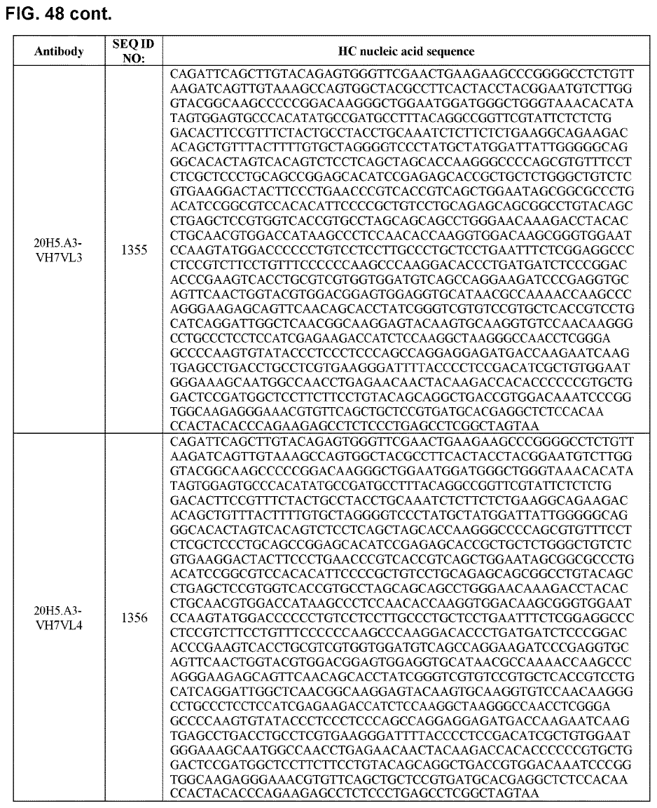

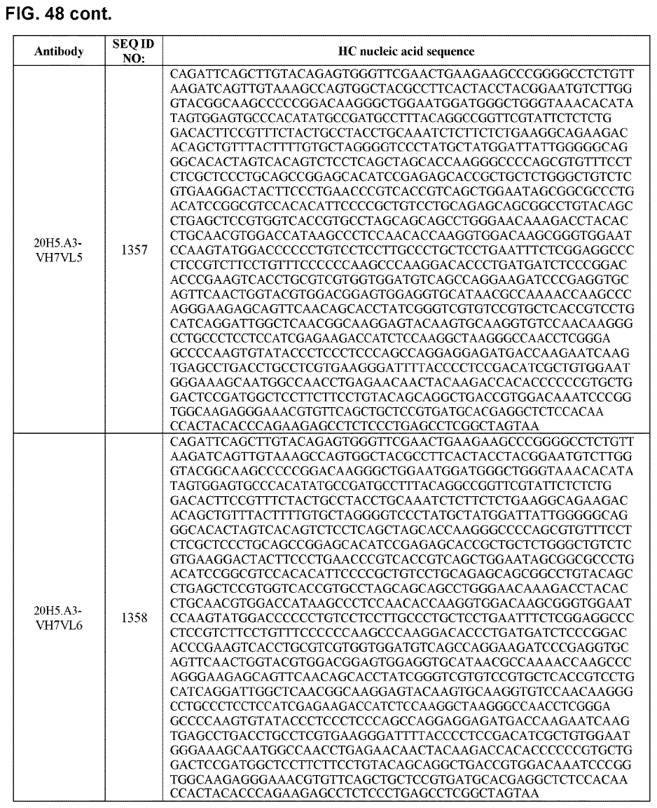

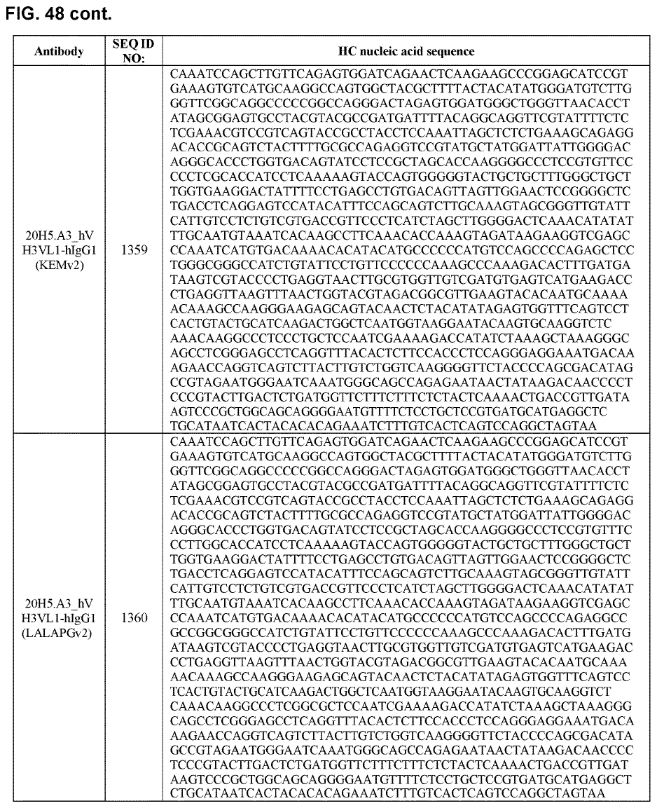

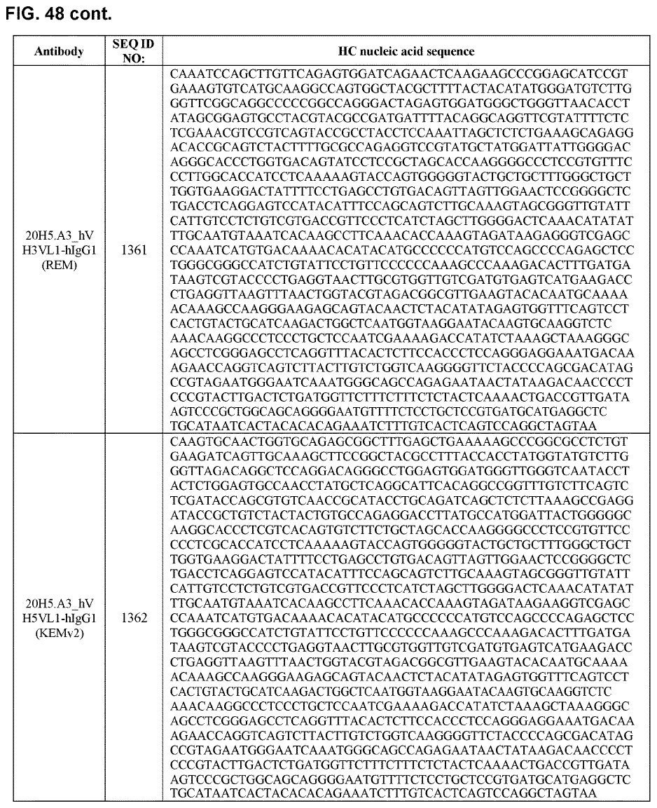

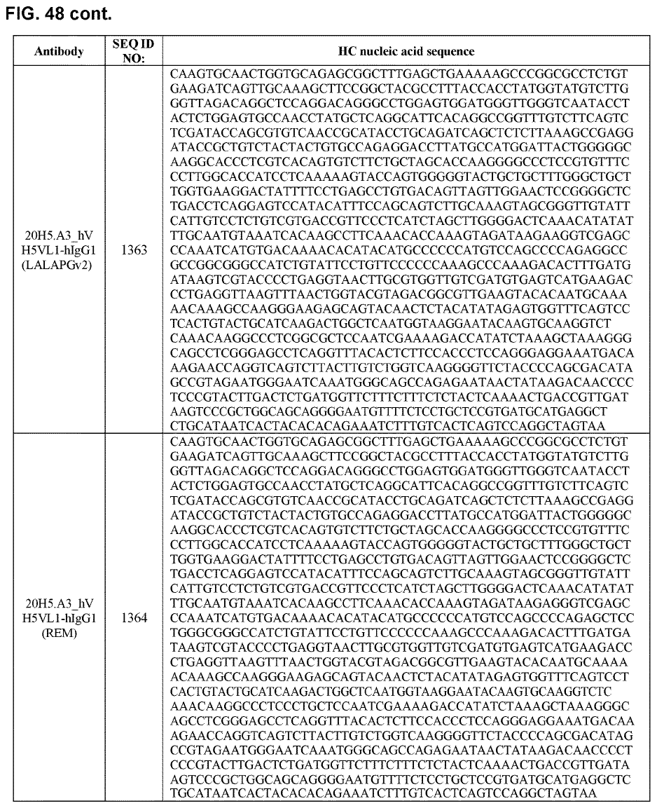

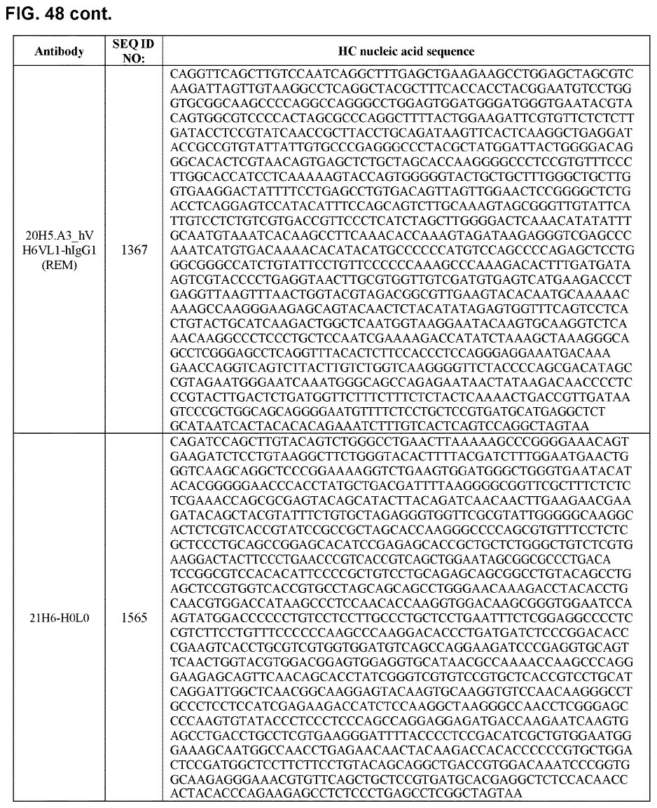

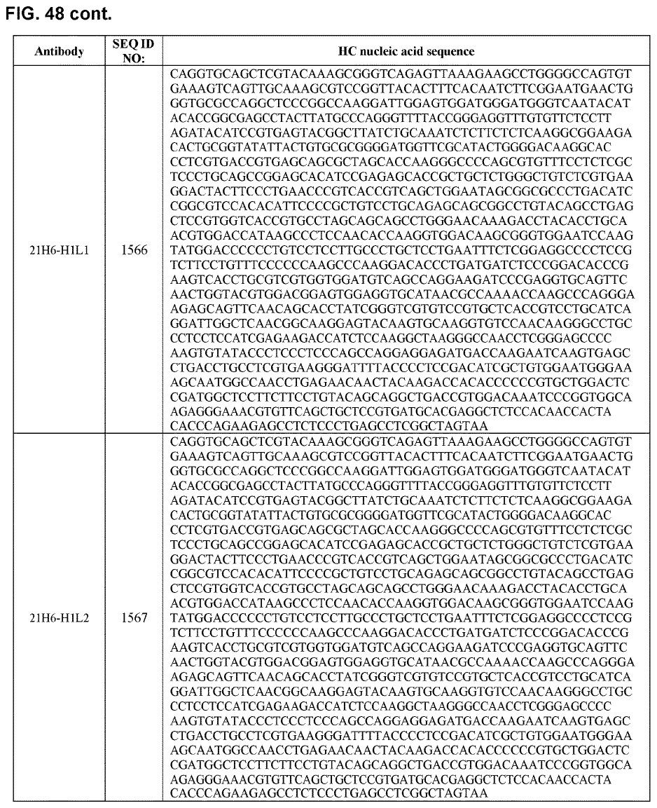

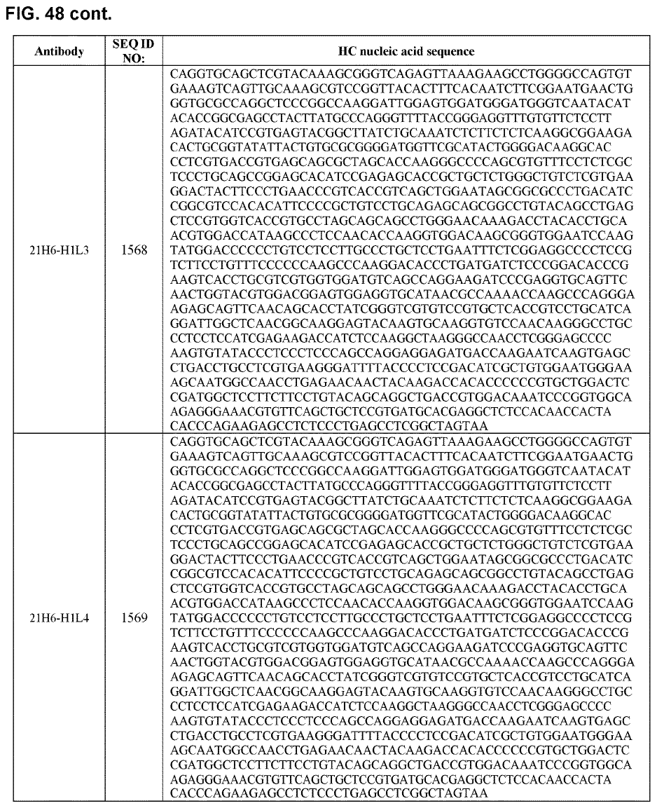

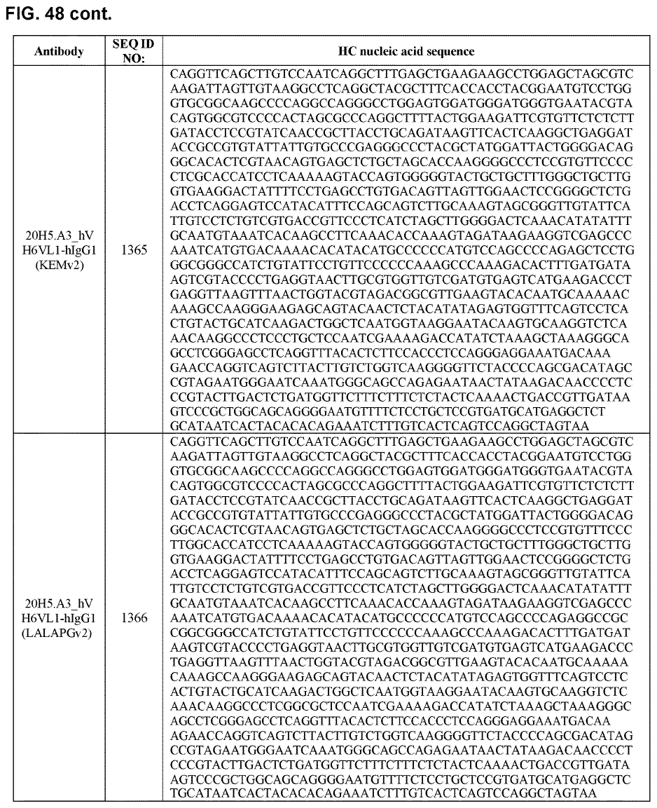

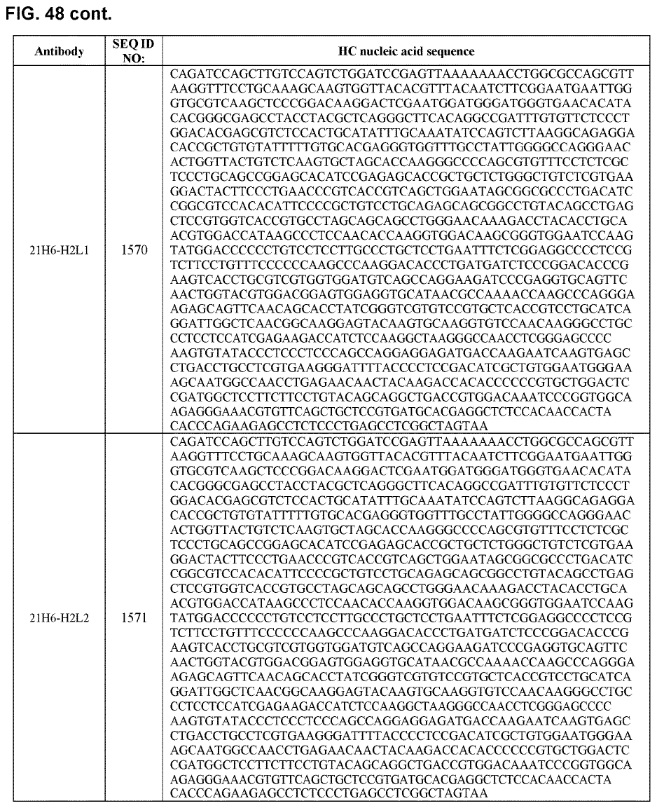

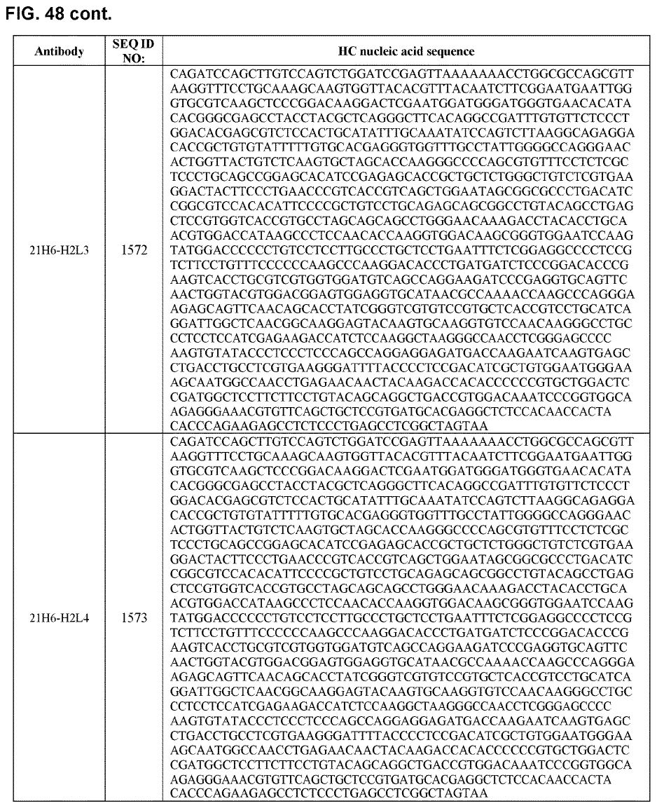

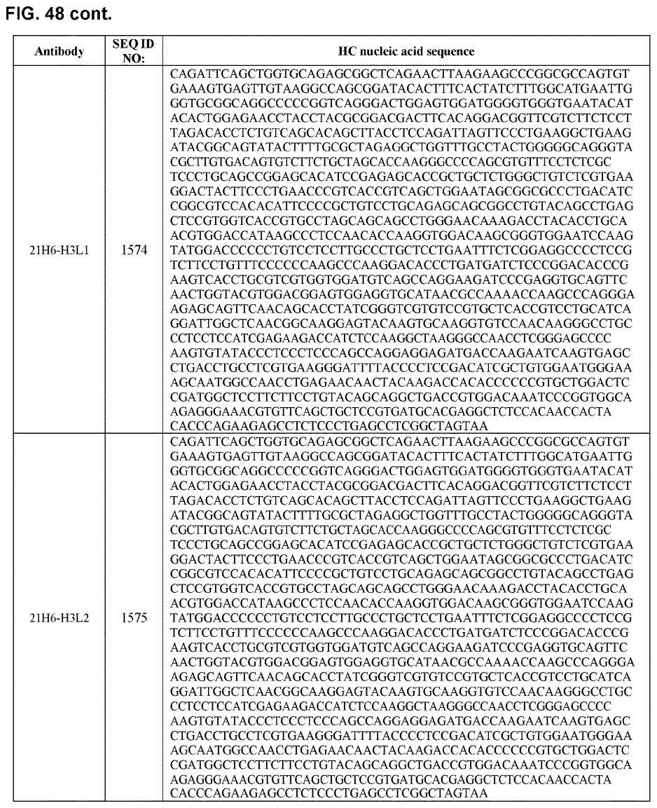

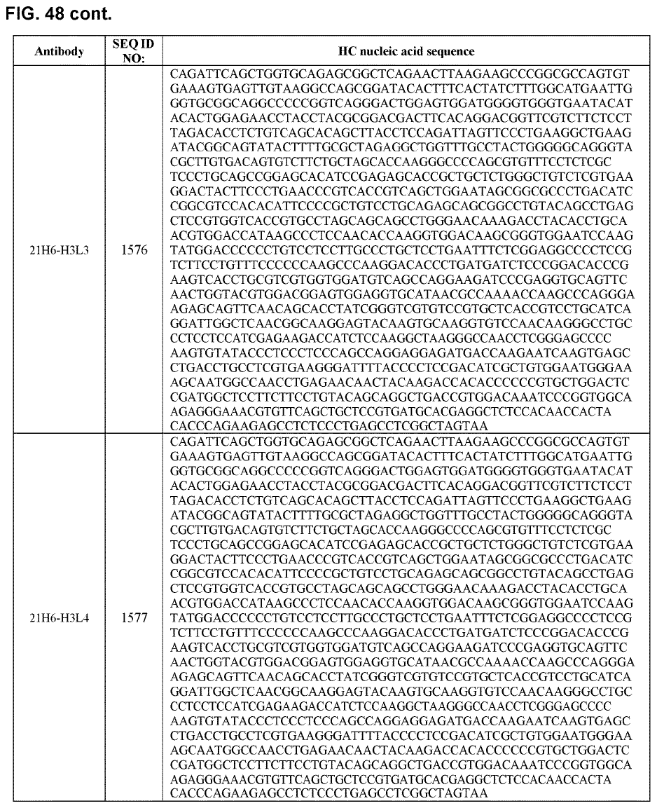

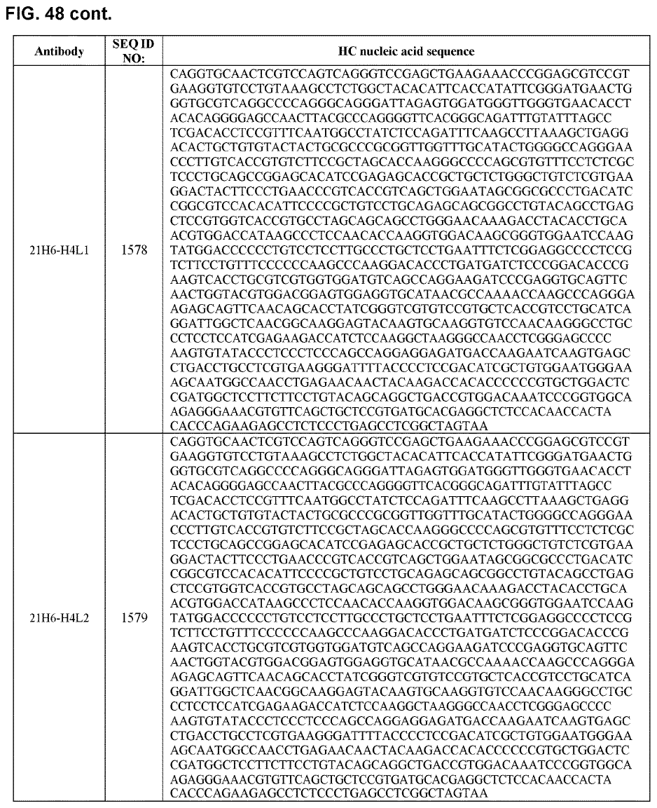

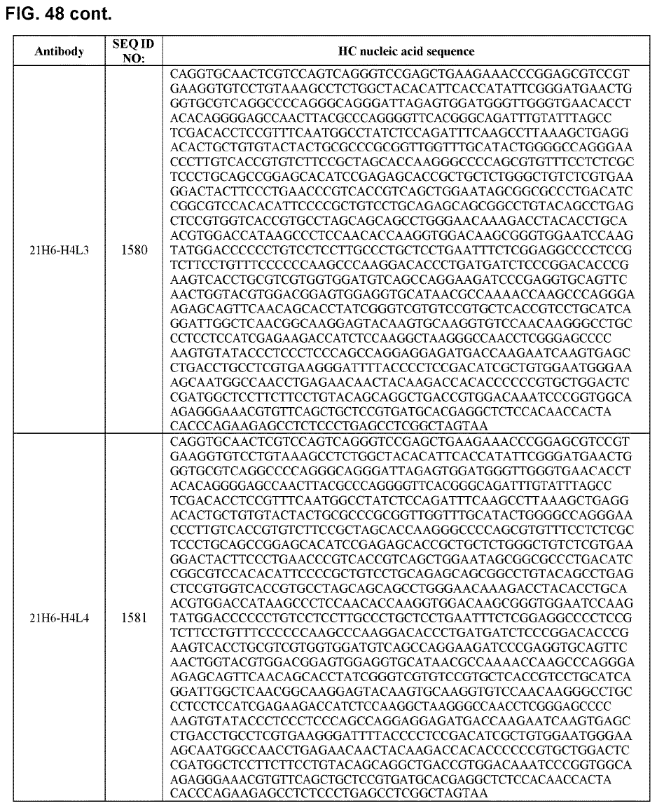

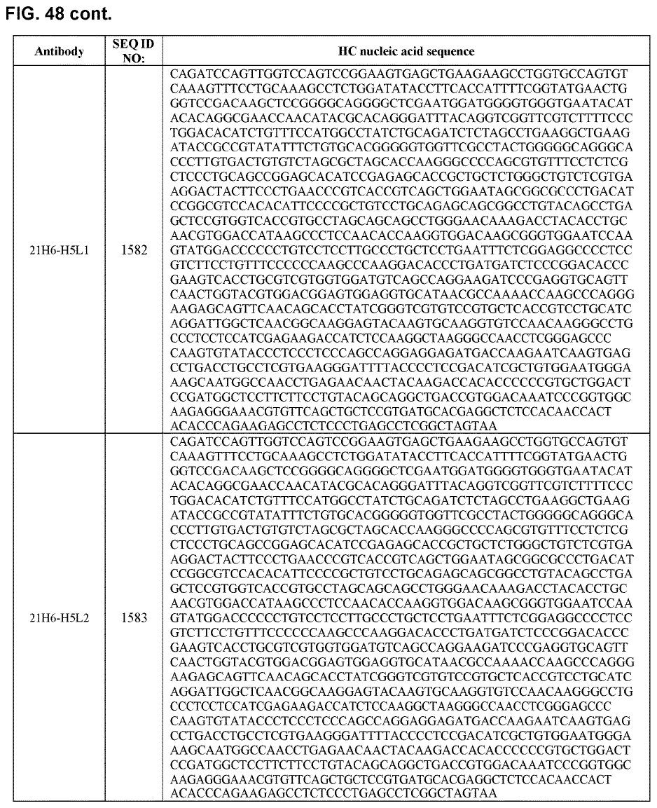

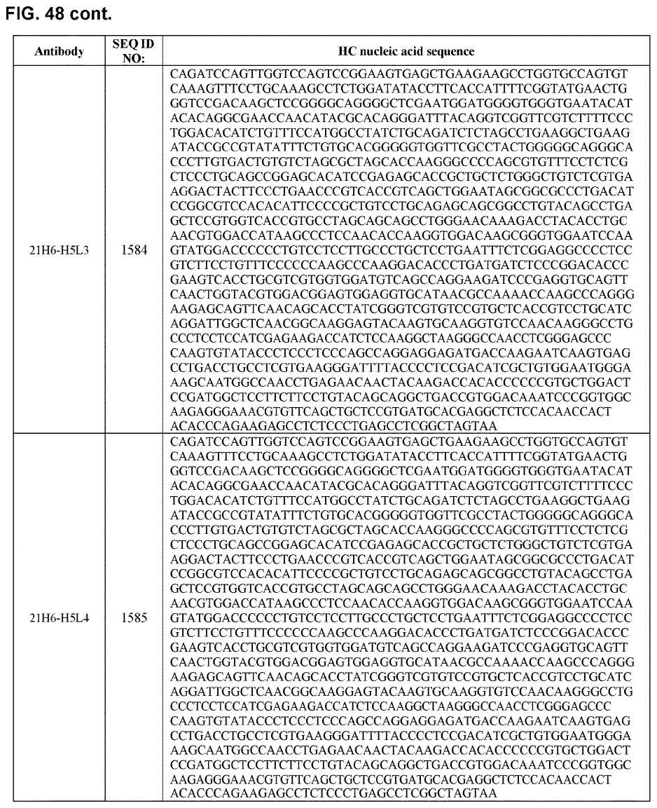

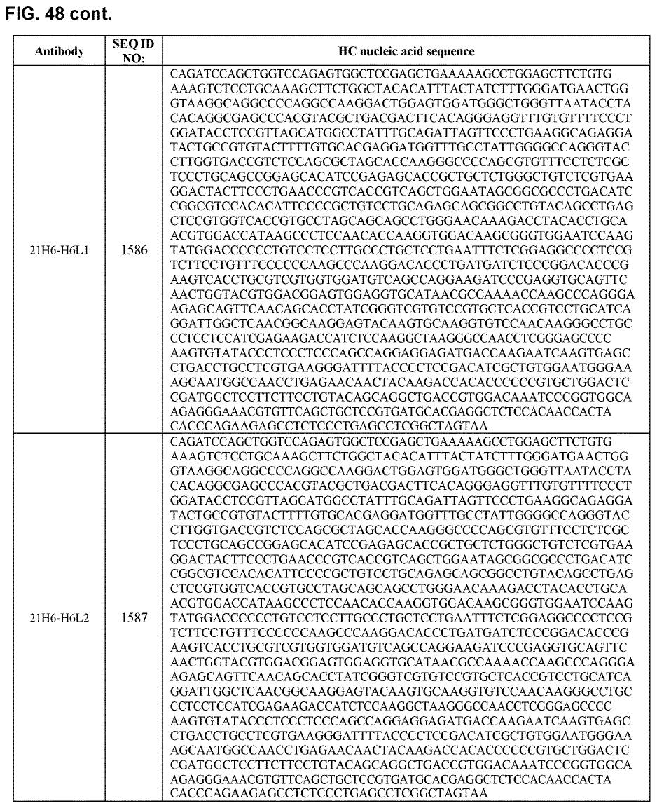

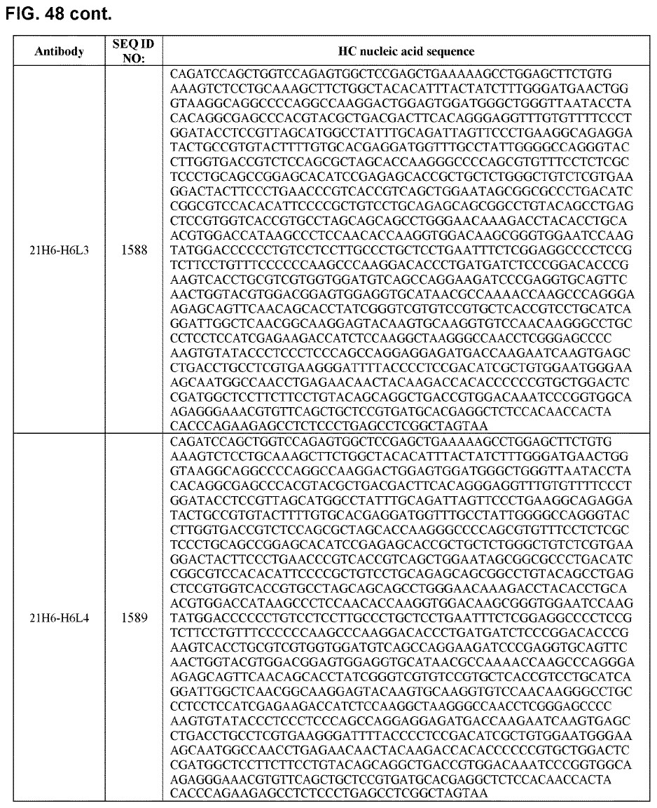

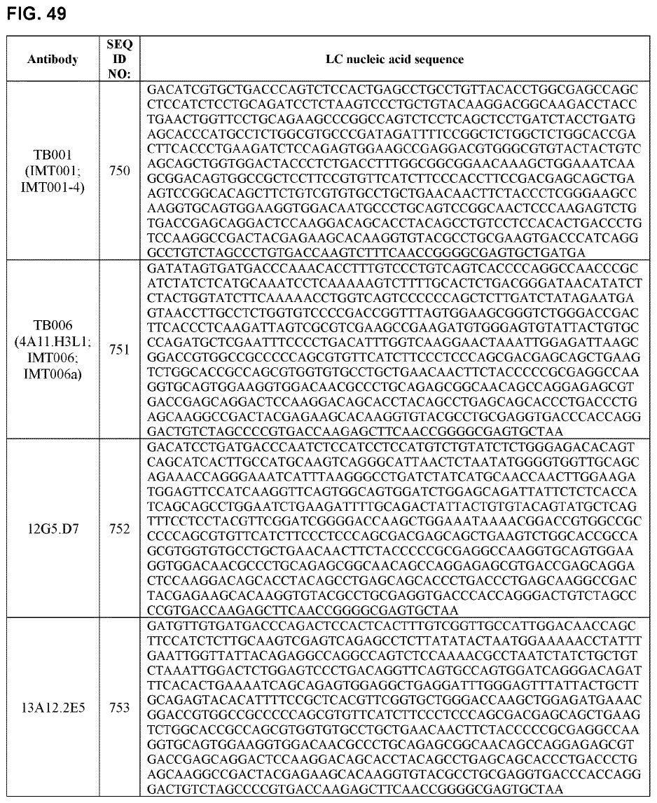

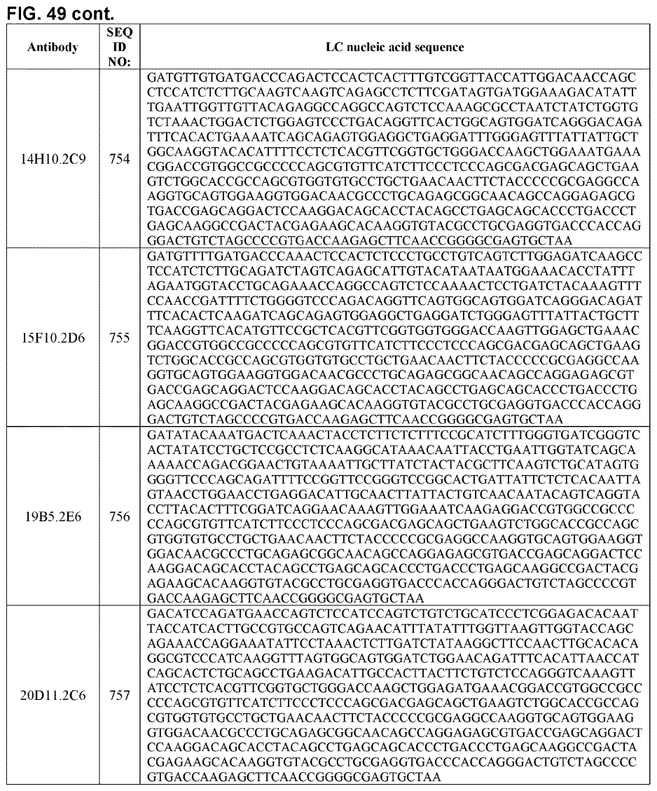

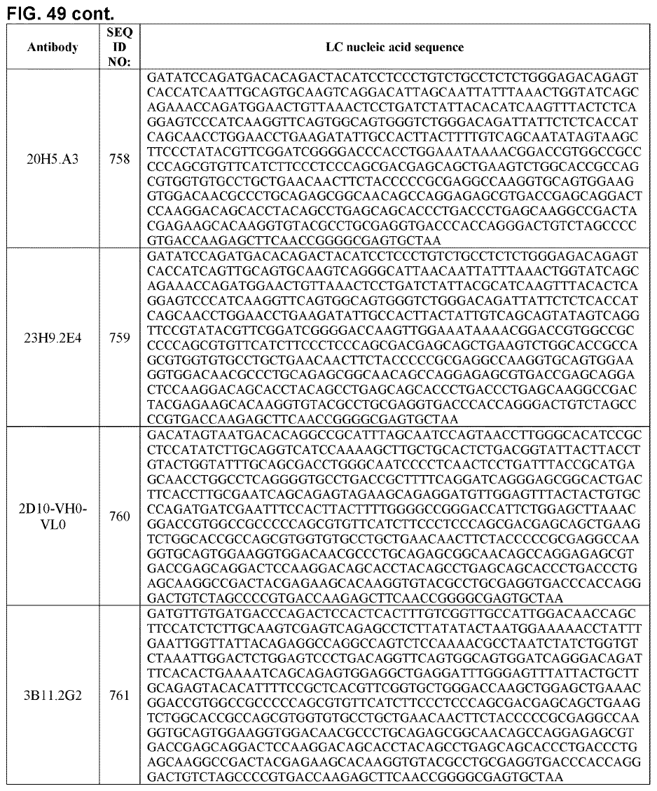

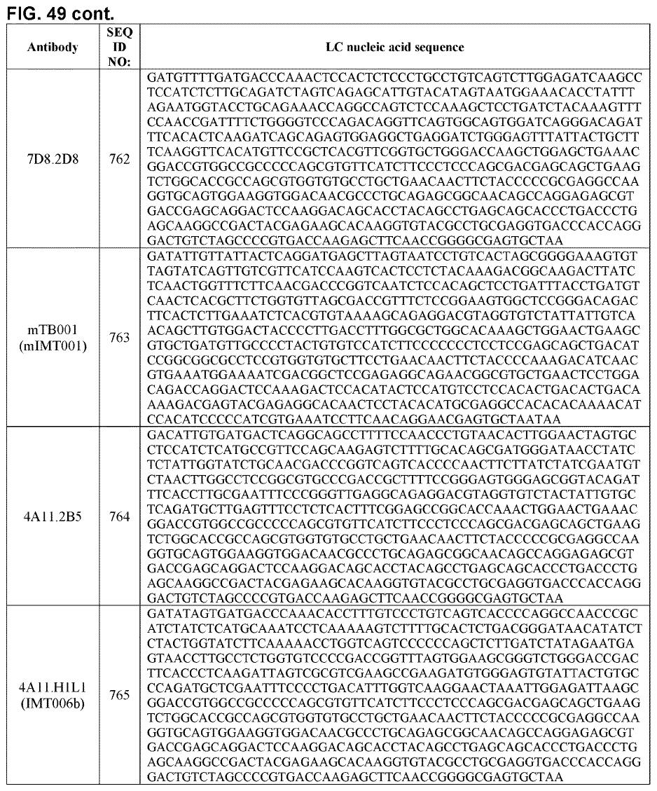

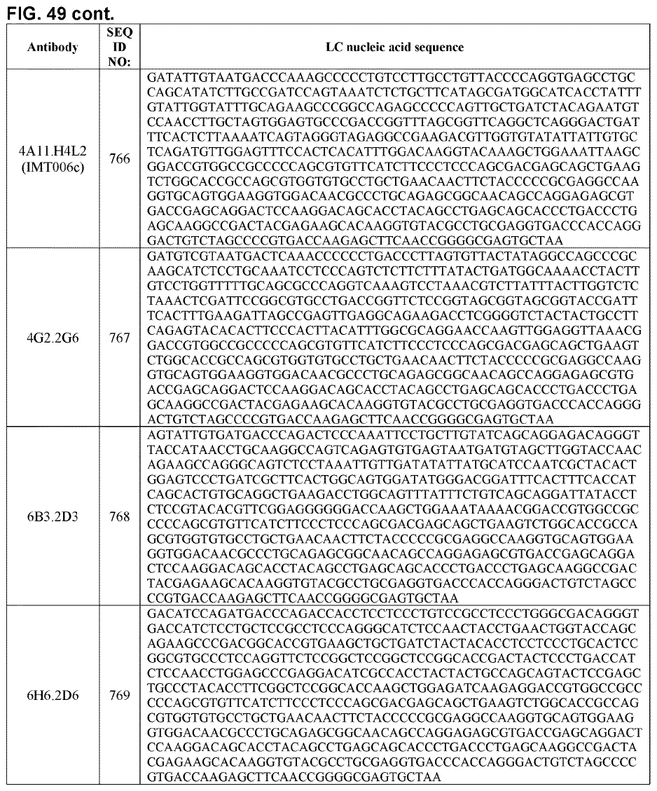

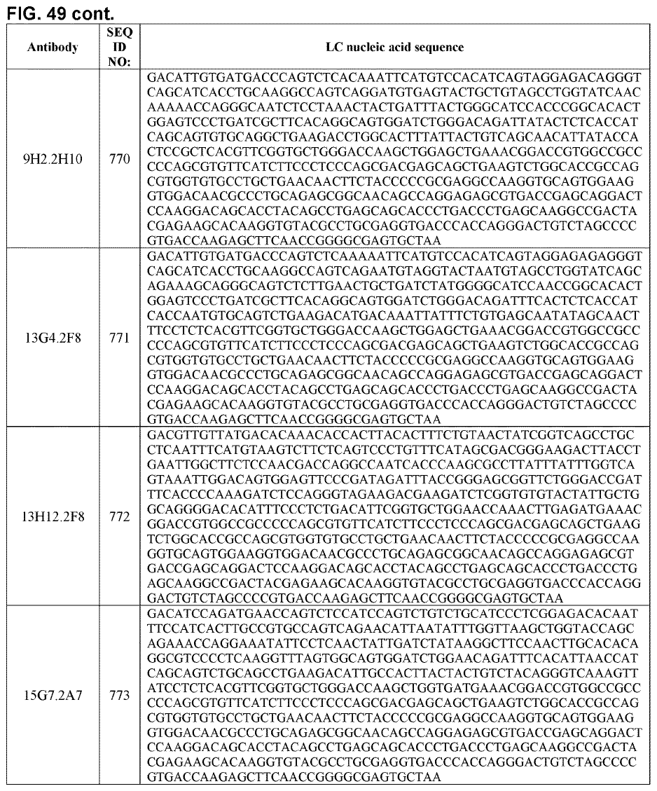

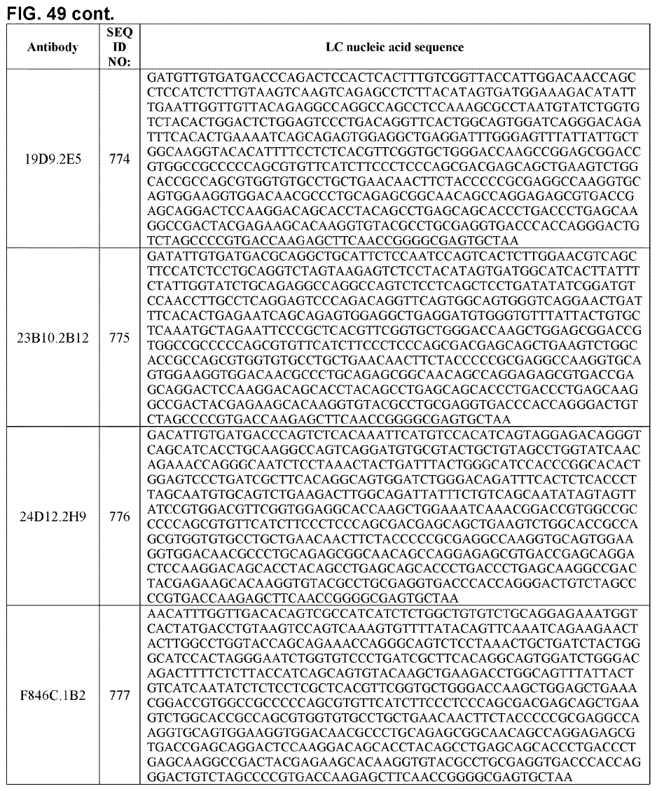

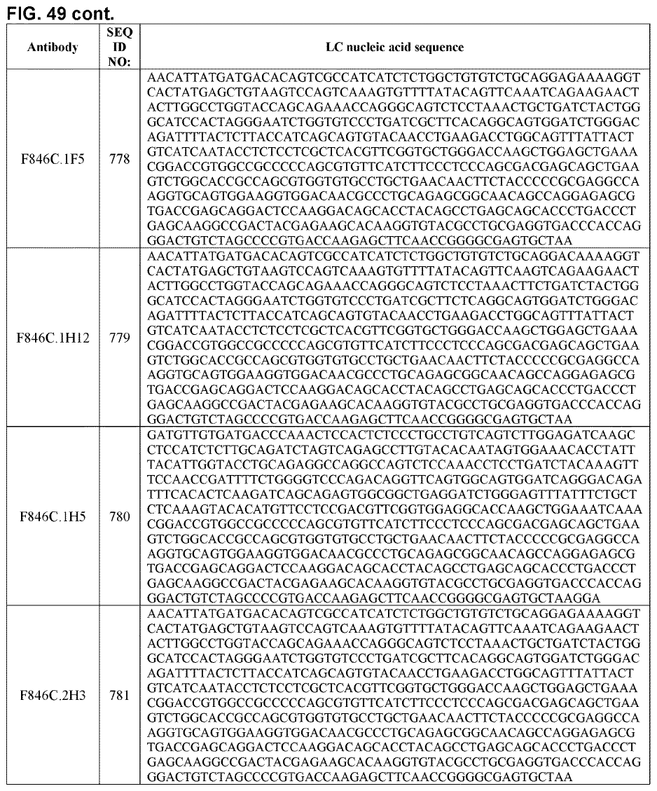

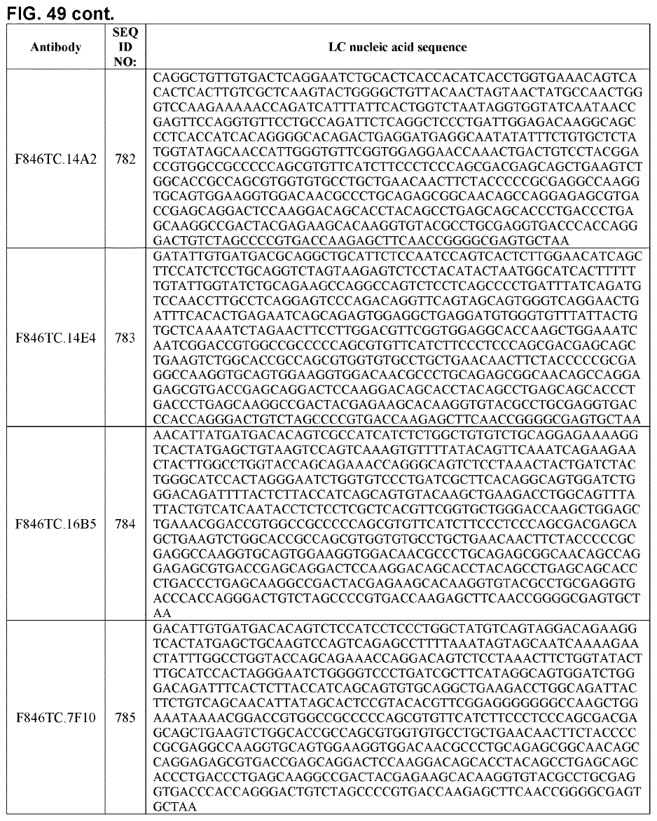

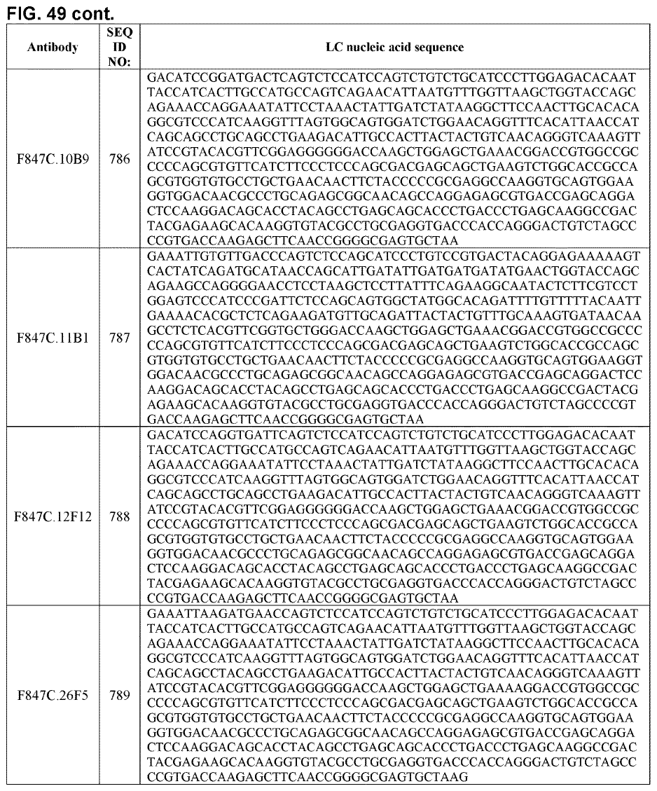

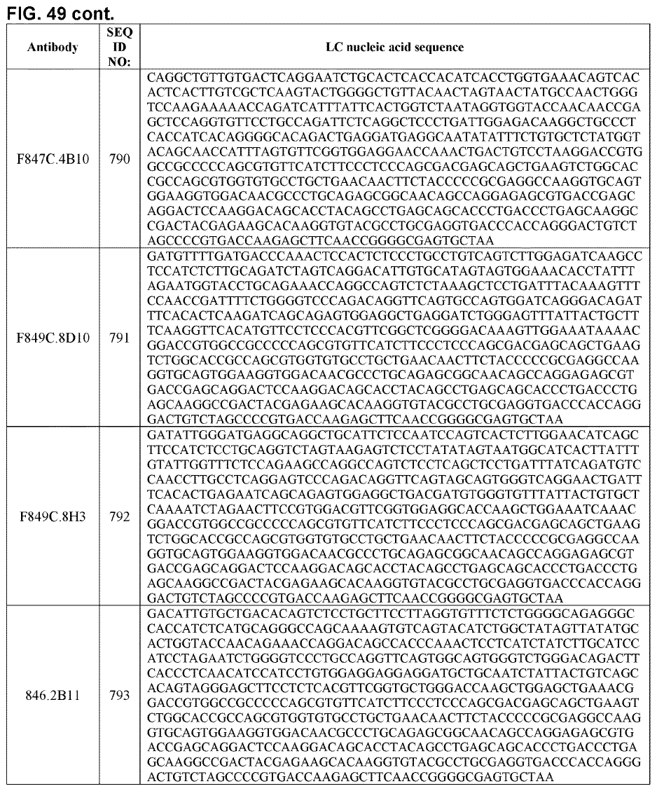

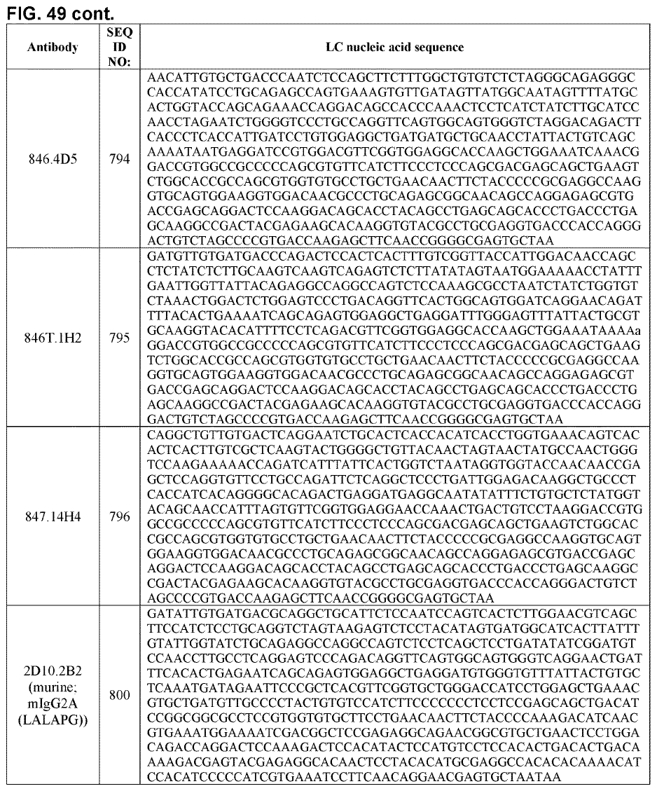

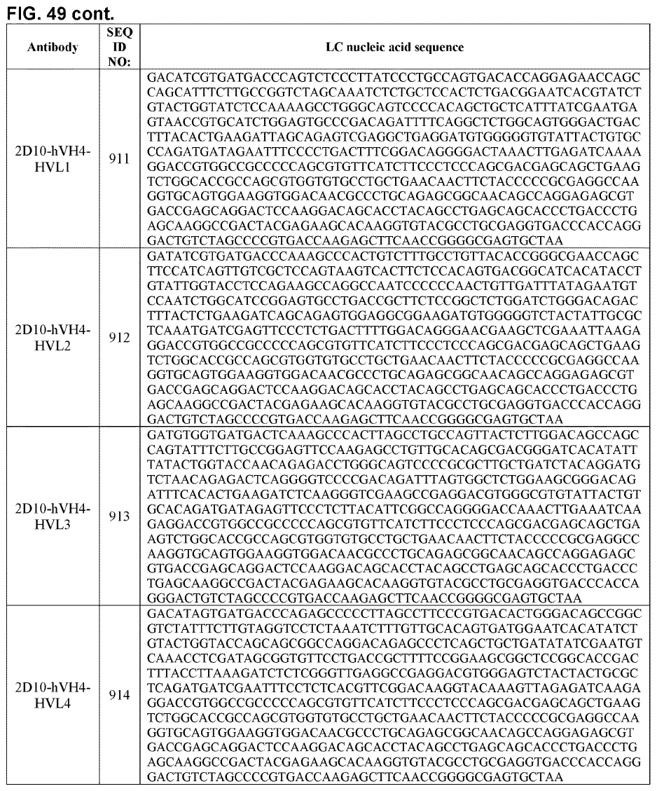

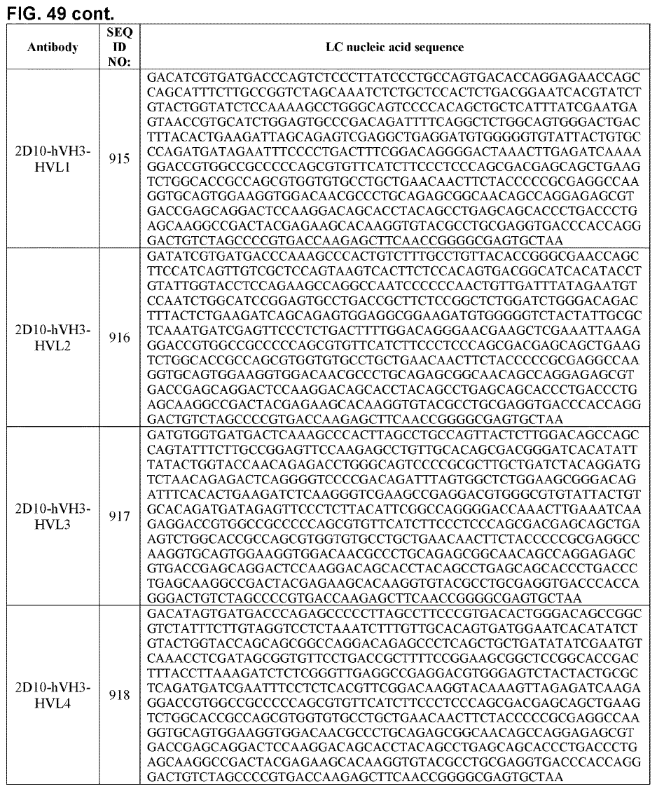

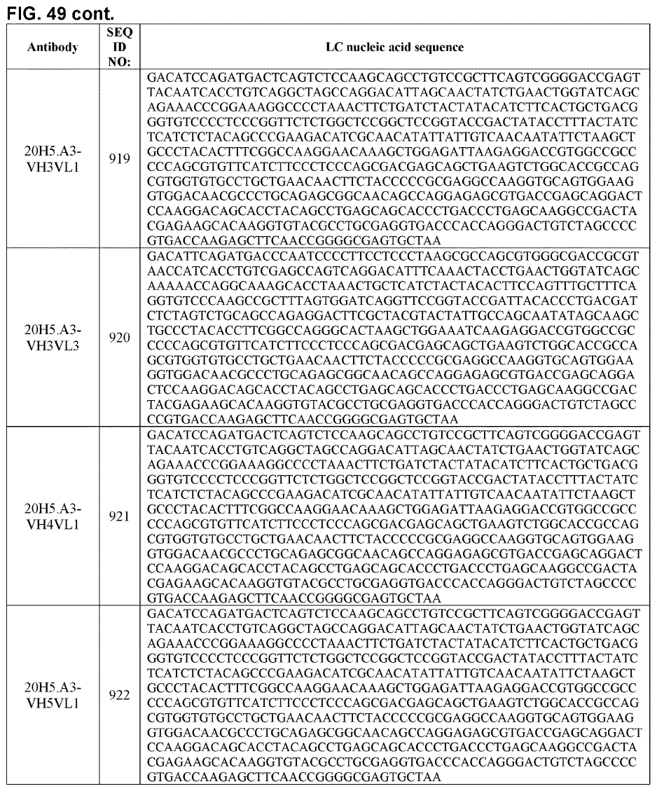

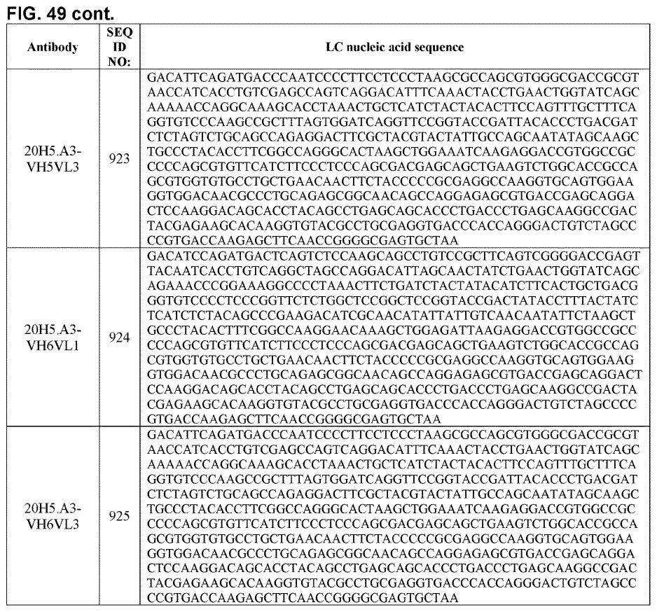

In some embodiments, any one or more of the constructs shown in any one or more of FIG. 35-50 can be used in any one or more of the embodiments provided herein (e.g., methods of treatment or uses in the preparation of a medicament for a therapy, etc.).

### Payload

In some embodiments, any anti-Gal3 antibody disclosed herein further comprises a payload (e.g., is attached to a payload). In some cases, the payload comprises a small molecule, a protein or functional fragment thereof, a peptide, or a nucleic acid polymer.

In some cases, the number of payloads conjugated to the anti-Gal3 antibody (e.g., the drug-to-antibody ratio or DAR) is about 1:1, one payload to one anti-Gal3 antibody. In some cases, the ratio of the payloads to the anti-Gal3 antibody is about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some cases, the ratio of the payloads to the anti-Gal3 antibody is about 2:1. In some cases, the ratio of the payloads to the anti-Gal3 antibody is about 3:1. In some cases, the ratio of the payloads to the anti-Gal3 antibody is about 4:1. In some cases, the ratio of the payloads to the anti-Gal3 antibody is about 6:1. In some cases, the ratio of the payloads to the anti-Gal3 antibody is about 8:1. In some cases, the ratio of the payloads to the anti-Gal3 antibody is about 12:1.

In some embodiment, the payload is a small molecule. In some instances, the small molecule is a cytotoxic payload. Exemplary cytotoxic payloads include, but are not limited to, microtubule disrupting agents, DNA modifying agents, or Akt inhibitors.

In some embodiments, the payload comprises a microtubule disrupting agent. Exemplary microtubule disrupting agents include, but are not limited to, 2-methoxyestradiol, auristatin, chalcones, colchicine, combretastatin, cryptophycin, dictyostatin, discodermolide, dolastain, eleutherobin, epothilone, halichondrin, laulimalide, maytansine, noscapinoid, paclitaxel, peloruside, phomopsin, podophyllotoxin, rhizoxin, spongistatin, taxane, tubulysin, vinca alkaloid, vinorelbine, or derivatives or analogs thereof.

In some embodiments, the maytansine is a maytansinoid. In some embodiments, the maytansinoid is DM1, DM4, or ansamitocin. In some embodiments, the maytansinoid is DM1. In some embodiments, the maytansinoid is DM4. In some embodiments, the maytansinoid is ansamitocin. In some embodiments, the maytansinoid is a maytansionid derivative or analog such as described in U.S. Pat. Nos. 5,208,020, 5,416,064, 7,276,497, and 6,716,821 or U.S. Publication Nos. 2013029900 and US20130323268.

In some embodiments, the payload is a dolastatin, or a derivative or analog thereof. In some embodiments, the dolastatin is dolastatin 10 or dolastatin 15, or derivatives or analogs thereof. In some embodiments, the dolastatin 10 analog is auristatin, soblidotin, symplostatin 1, or symplostatin 3. In some embodiments, the dolastatin 15 analog is cemadotin or tasidotin.

In some embodiments, the dolastatin 10 analog is auristatin or an auristatin derivative. In some embodiments, the auristatin or auristatin derivative is auristatin E (AE), auristatin F (AF), auristatin E5-benzoylvaleric acid ester (AEVB), monomethyl auristatin E (MMAE), monomethyl auristatin F (MMAF), or monomethyl auristatin D (MMAD), auristatin PE, or auristatin PYE. In some embodiments, the auristatin derivative is monomethyl auristatin E (MMAE). In some embodiments, the auristatin derivative is monomethyl auristatin F (MMAF). In some embodiments, the auristatin is an auristatin derivative or analog such as described in U.S. Pat. Nos. 6,884,869, 7,659,241, 7,498,298, 7,964,566, 7,750,116, 8,288,352, 8,703,714, and 8,871,720.

In some embodiments, the payload comprises a DNA modifying agent. In some embodiments, the DNA modifying agent comprises DNA cleavers, DNA intercalators, DNA transcription inhibitors, or DNA cross-linkers. In some instances, the DNA cleaver comprises bleomycin A2, calicheamicin, or derivatives or analogs thereof. In some instances, the DNA intercalator comprises doxorubicin, epirubicin, PNU-159682, duocarmycin, pyrrolobenzodiazepine, oligomycin C, daunorubicin, valrubicin, topotecan, or derivatives or analogs thereof. In some instances, the DNA transcription inhibitor comprises dactinomycin. In some instances, the DNA cross-linker comprises mitomycin C.

In some embodiments, the DNA modifying agent comprises amsacrine, anthracycline, camptothecin, doxorubicin, duocarmycin, enediyne, etoposide, indolinobenzodiazepine, netropsin, teniposide, or derivatives or analogs thereof.

In some embodiments, the anthracycline is doxorubicin, daunorubicin, epirubicin, idarubicin, mitomycin-C, dactinomycin, mithramycin, nemorubicin, pixantrone, sabarubicin, or valrubicin.

In some embodiments, the analog of camptothecin is topotecan, irinotecan, silatecan, cositecan, exatecan, lurtotecan, gimatecan, belotecan, rubitecan, or SN-38.

In some embodiments, the duocarmycin is duocarmycin A, duocarmycin B1, duocarmycin B2, duocarmycin C1, duocarmycin C2, duocarmycin D, duocarmycin SA, or CC-1065. In some embodiments, the enediyne is a calicheamicin, esperamicin, or dynemicin A.

In some embodiments, the pyrrolobenzodiazepine is anthramycin, abbeymycin, chicamycin, DC-81, mazethramycin, neothramycins A, neothramycin B, porothramycin, prothracarcin, sibanomicin (DC-102), sibiromycin, or tomaymycin. In some embodiments, the pyrrolobenzodiazepine is a tomaymycin derivative, such as described in U.S. Pat. Nos. 8,404,678 and 8,163,736. In some embodiments, the pyrrolobenzodiazepine is such as described in U.S. Pat. Nos. 8,426,402, 8,802,667, 8,809,320, 6,562,806, 6,608,192, 7,704,924, 7,067,511, 7,612,062, 7,244,724, 7,528,126, 7,049,311, 8,633,185, 8,501,934, and 8,697,688 and U.S. Publication No. US20140294868.

In some embodiments, the pyrrolobenzodiazepine is a pyrrolobenzodiazepine dimer. In some embodiments, the PBD dimer is a symmetric dimer. Examples of symmetric PBD dimers include, but are not limited to, SJG-136 (SG-2000), ZC-423 (SG2285), SJG-720, SJG-738, ZC-207 (SG2202), and DSB-120. In some embodiments, the PBD dimer is an unsymmetrical dimer. Examples of unsymmetrical PBD dimers include, but are not limited to, SJG-136 derivatives such as described in U.S. Pat. Nos. 8,697,688 and 9,242,013 and U.S. Publication No. 20140286970.

In some embodiments, the payload comprises an Akt inhibitor. In some cases, the Akt inhibitor comprises ipatasertib (GDC-0068) or derivatives thereof.

In some embodiments, the payload comprises a polymerase inhibitor, including, but not limited to polymerase II inhibitors such as a-amanitin, and poly(ADP-ribose) polymerase (PARP) inhibitors. Exemplary PARP inhibitors include, but are not limited to Iniparib (BSI 201), Talazoparib (BMN-673), Olaparib (AZD-2281), Olaparib, Rucaparib (AG014699, PF-01367338), Veliparib (ABT-888), CEP 9722, MK 4827, BGB-290, or 3-aminobenzamide.

In some embodiments, the payload comprises a detectable moiety. As used herein, a “detectable moiety” may comprise an atom, molecule, or compound that is useful in diagnosing, detecting or visualizing a location and/or quantity of a target molecule, cell, tissue, organ, and the like. Detectable moieties that can be used in accordance with the embodiments herein include, but are not limited to, radioactive substances (e.g. radioisotopes, radionuclides, radiolabels or radiotracers), dyes, contrast agents, fluorescent compounds or molecules, bioluminescent compounds or molecules, enzyme and enhancing agents (e.g. paramagnetic ions), or specific binding moieties such as streptavidin, avidin, or biotin. In addition, some nanoparticles, for example quantum dots or metal nanoparticles can be suitable for use as a detectable moiety.

Exemplary radioactive substances that can be used as detectable moieties in accordance with the embodiments herein include, but are not limited to, ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, 189Re, ¹⁹⁴Ir, ¹⁹⁸Au. ¹⁹⁹Au, ²¹¹At, ²¹¹Pb, ²¹²Bi, ²¹²Pb, ²¹³Bi, ²²³Ra and ²²⁵Ac. Exemplary paramagnetic ions substances that can be used as detectable markers include, but are not limited to ions of transition and lanthanide metals (e.g. metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the detectable marker is a radioactive metal or paramagnetic ion, in some embodiments, the marker can be reacted with a reagent having a long tail with one or more chelating groups attached to the long tail for binding these ions. The long tail can be a polymer such as a polylysine, polysaccharide, or other derivatized or derivatizable chain having pendant groups to which may be bound to a chelating group for binding the ions. Examples of chelating groups that may be used according to the embodiments herein include, but are not limited to, ethylenediaminetetraacetic acid (EDTA), diethylenetriaminepentaacetic acid (DTPA), DOTA, NOTA, NOGADA, NETA, deferoxamine (DfO), porphyrins, polyamines, crown ethers, bis-thiosemicarbazones, polyoximes, and like groups. The chelate can be linked to the antigen binding construct by a group which allows formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antigen binding constructs and carriers described herein. Macrocyclic chelates such as NOTA, NOGADA, DOTA, and TETA are of use with a variety of metals and radiometals including, but not limited to, radionuclides of gallium, yttrium and copper, respectively. Other ring-type chelates such as macrocyclic polyethers, which are of interest for stably binding radionuclides, such as Radium-223 for RAIT may be used. In certain embodiments, chelating moieties may be used to attach a PET imaging agent, such as an Aluminum-18F complex, to a targeting molecule for use in PET analysis.

Exemplary contrast agents that can be used as detectable moieties in accordance with the embodiments of the disclosure include, but are not limited to, barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, thallous chloride, or combinations thereof.

Bioluminescent and fluorescent compounds or molecules and dyes that can be used as detectable moieties in accordance with the embodiments of the disclosure include, but are not limited to, allophycocyanin (APC), phycoerythrin (PE), fluorescein, fluorescein isothiocyanate (FITC), OREGON GREEN™, rhodamine, Texas red, tetrarhodimine isothiocynate (TRITC), Cy3, Cy5, and the like), fluorescent markers (e.g., green fluorescent protein (GFP) and the like), autoquenched fluorescent compounds that are activated by tumor-associated proteases, enzymes (e.g., luciferase, horseradish peroxidase, alkaline phosphatase, and the like), nanoparticles, biotin, digoxigenin or combinations thereof.

Enzymes that can be used as detectable moieties in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

In some embodiments, the payload is a nanoparticle. The term “nanoparticle” refers to a microscopic particle whose size is measured in nanometers, e.g., a particle with at least one dimension less than about 100 nm. Nanoparticles can be used as detectable substances because they are small enough to scatter visible light rather than absorb it. For example, gold nanoparticles possess significant visible light extinction properties and appear deep red to black in solution. As a result, compositions comprising antigen binding constructs conjugated to nanoparticles can be used for the in vivo imaging of T-cells in a subject. At the small end of the size range, nanoparticles are often referred to as clusters. Metal, dielectric, and semiconductor nanoparticles have been formed, as well as hybrid structures (e.g. core-shell nanoparticles). Nanospheres, nanorods, and nanocups are just a few of the shapes that have been grown. Semiconductor quantum dots and nanocrystals are examples of additional types of nanoparticles. Such nanoscale particles can be used as payloads to be conjugated to any one of the anti-Gal3 antibodies disclosed herein.

In some embodiments, the payload is an antimicrobial agent, a therapeutic agent, a prodrug, a peptide, a protein, an enzyme, a lipid, a biological response modifier, a pharmaceutical agent, a lymphokine, a heterologous antibody or fragment thereof, a detectable label, a polyethylene glycol (PEG) molecule, or a combination of two or more of the agents. In some embodiments, the payload comprises a neuroactive polypeptide, for example, a neurotrophic factors, endocrine factors, growth factors, paracrine factors, hypothalamic release factors, neurotransmitter polypeptides, polypeptide agonists for a receptor expressed by a CNS cell, polypeptides involved in lysosomal storage disease or any combination thereof. In some embodiments, the payload comprises an IL-1 receptor antagonist (IL-IRa), dalargin, an interferon-β, Glial-derived neurotrophic factor (GDNF), tumor necrosis factor receptor (TNFR), nerve growth factor (NGF), brain derived neurotrophic factor (BDNF), neurotrophin-4/5, neurotrophin (NT)-3, a neurturin, neuregulin, a netrin, ciliary neurotrophic factor (CNTF), stem cell factor (SCF), a semaphorin, hepatocyte growth factor (HGF), epidermal growth factor (EGF), transforming growth factor (TGF)-cx, TGF-β, vascular endothelial growth factor (VEGF), platelet-derived growth factor (PDGF), heregulin, artemin, persephin, interleukins, granulocyte-colony stimulating factor (CSF), granulocyte-macrophage-CSF, cardiotrophin-1, hedgehogs, leukemia inhibitory factor (LIF), midkine, pleiotrophin, erythropoictin (EPO), bone morphogenetic proteins (BMPs), netrins, saposins, any fragment thereof, or any combination thereof. In some embodiments, the payload is another antibody, or a heavy and/or light chain, or any other fragment thereof.

In some embodiments, the payload comprises a heterologous antibody or fragment thereof, for example, a heterologous antibody or fragment thereof specifically binds to one or more of beta-secretase 1 (BACE1), CD20, CD25, CD52, CD33, CTLA-4, tenascin, alpha-4 (a4) integrin, IL-12, IL-23, the p40 subunit of IL-12/IL-23, amyloid-13 (AI3), Huntingtin, nerve growth factor (NGF), epidermal growth factor receptor (EGFR/HER1), human epidermal growth factor receptor 2 (HER2/neu), vascular endothelial growth factor (VEGF), TrkA, TNF-α, TNF-β, a-synuclein Tau, apolipoprotein E4 (ApoE4), prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), caspase 6, a neurotrophic factor and/or a neurotrophic factor receptor.

In some embodiments, the payload comprises an immunomodulatory agent. Useful immunomodulatory agents include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4 (5)-imidazoles, 4-hydroxytamoxifen, trioxifene, keoxifene, LY 117018, onapristone, and toremifene; and anti-androgens such as flutamide, nilutamide, bicalutamide, leuprolide, and goserelin; and anti-adrenal agents. Illustrative immunosuppressive agents include, but are not limited to 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, streptokinase, or rapamycin.

In some embodiments, the payload comprises an immune modulator. Exemplary immune modulators include, but are not limited to, gancyclovir, etanercept, tacrolimus, sirolimus, voclosporin, cyclosporine, rapamycin, cyclophosphamide, azathioprine, mycophenolate mofetil, methotrexate, glucocorticoid and its analogs, xanthines, stem cell growth factors, lymphotoxins, hematopoietic factors, tumor necrosis factor (TNF) (e.g., TNFα), interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-alpha, interferon-beta, interferon-gamma), the stem cell growth factor designated “S1 factor,” erythropoietin and thrombopoietin, or a combination thereof.

In some embodiments, the payload comprises an immunotoxin. Immunotoxins include, but are not limited to, ricin, radionuclides, pokeweed antiviral protein, Pseudomonas exotoxin A, diphtheria toxin, ricin A chain, fungal toxins such as restriction and phospholipase enzymes. See, generally, “Chimeric Toxins,” Olsnes and Pihl, Pharmac. Ther. 15:355-381 (1981); and “Monoclonal Antibodies for Cancer Detection and Therapy,” eds. Baldwin and Byers, pp. 159-179, 224-266, Academic Press (1985).

In some instances, the payload comprises a nucleic acid polymer. In such instances, the nucleic acid polymer comprises short interfering nucleic acid (siNA), short interfering RNA (siRNA), double-stranded RNA (dsRNA), micro-RNA (miRNA), short hairpin RNA (shRNA), an antisense oligonucleotide. In other instances, the nucleic acid polymer comprises an mRNA, encoding, e.g., a cytotoxic protein or peptide or an apoptotic triggering protein or peptide. Exemplary cytotoxic proteins or peptides include a bacterial cytotoxin such as an alpha-pore forming toxin (e.g., cytolysin A from E. coli), a beta-pore-forming toxin (e.g., α-Hemolysin, PVL—panton Valentine leukocidin, aerolysin, clostridial Epsilon-toxin, Clostridium perfringens enterotoxin), binary toxins (anthrax toxin, edema toxin, C. botulinum C2 toxin, C spirofome toxin, C. perfringens iota toxin, C. difficile cyto-lethal toxins (A and B)), prion, parasporin, a cholesterol-dependent cytolysins (e.g., pneumolysin), a small pore-forming toxin (e.g., Gramicidin A), a cyanotoxin (e.g., microcystins, nodularins), a hemotoxin, a neurotoxin (e.g., botulinum neurotoxin), a cytotoxin, cholera toxin, diphtheria toxin, Pseudomonas exotoxin A, tetanus toxin, or an immunotoxin (idarubicin, ricin A, CRM9, Pokeweed antiviral protein, DT). Exemplary apoptotic triggering proteins or peptides include apoptotic protease activating factor-1 (Apaf-1), cytochrome-c, caspase initiator proteins (CASP2, CASP8, CASP9, CASP10), apoptosis inducing factor (AIF), p53, p73, p63, Bcl-2, Bax, granzyme B, poly-ADP ribose polymerase (PARP), and P 21-activated kinase 2 (PAK2). In additional instances, the nucleic acid polymer comprises a nucleic acid decoy. In some instances, the nucleic acid decoy is a mimic of protein-binding nucleic acids such as RNA-based protein-binding mimics. Exemplary nucleic acid decoys include transactivating region (TAR) decoy and Rev response element (RRE) decoy.

In some cases, the payload is an aptamer. Aptamers are small oligonucleotide or peptide molecules that bind to specific target molecules. Exemplary nucleic acid aptamers include DNA aptamers, RNA aptamers, or XNA aptamers which are RNA and/or DNA aptamers comprising one or more unnatural nucleotides. Exemplary nucleic acid aptamers include ARC19499 (Archemix Corp.), REG1 (Regado Biosciences), and ARC1905 (Ophthotech).

Nucleic acids in accordance with the embodiments described herein optionally include naturally occurring nucleic acids, or one or more nucleotide analogs or have a structure that otherwise differs from that of a naturally occurring nucleic acid. For example, 2′-modifications include halo, alkoxy, and allyloxy groups. In some embodiments, the 2′-OH group is replaced by a group selected from H, OR, R, halo, SH, SR, NH₂, NHR, NR₂ or CN, wherein R is C₁-C₆ alkyl, alkenyl, or alkynyl, and halo is F, Cl, Br, or I. Examples of modified linkages include phosphorothioate and 5′-N-phosphoramidite linkages.

Nucleic acids having a variety of different nucleotide analogs, modified backbones, or non-naturally occurring internucleoside linkages are utilized in accordance with the embodiments described herein. In some cases, nucleic acids include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, O(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some cases, nucleic acids comprising such modifications display enhanced properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Different nucleotide modifications and/or backbone structures may exist at various positions in the nucleic acid. Such modifications include morpholinos, peptide nucleic acids (PNAs), methylphosphonate nucleotides, thiolphosphonate nucleotides, 2′-fluoro N3-P5′-phosphoramidites, 1′,5′-anhydrohexitol nucleic acids (HNAs), or a combination thereof.

Any of the anti-Gal3 antibodies disclosed herein may be conjugated to one or more (e.g. 1, 2, 3, 4, 5, 6, 7, 8, 9, or 10, or more) payloads described herein.

### Diagnostic Antibodies and Methods of Diagnosis

In some embodiments, anti-Gal3 diagnostic antibodies are disclosed. In some embodiments, the diagnostic antibody comprises an anti-Gal3 antibody comprising a detectable moiety. In some embodiments, the detectable moiety comprise radioactive material.

In some embodiments, diagnostic antibody is selected for use in diagnosing one or more neurological disorders. In some embodiments, the neurological disorder has a physical cause, such as external or internal mechanical trauma (e.g. stroke or concussion), biological trauma (e.g. infection), chemical trauma (e.g. toxins or drugs), aging and age-related senescence, genetics, and many other causes. In some embodiments, the neurological disorder is caused by the effect or accumulation of mutated or misfolded proteins. In some embodiments, the neurological disorder comprises inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer, or otherwise known by a person skilled in the art. Some neurological disorders can also be categorized as protcopathics.

In some embodiments, the diagnostic antibody is selected for use in diagnosing one or more proteopathies. In some embodiments, the proteopathy includes Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, tauopathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Friedreich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or otherwise known by a person skilled in the art, or any combination thereof.

In some embodiments, an anti-Gal3 antibody conjugated to a radioactive substance for use in detecting and/or diagnosing a subject as having, or at risk of having, a disease is disclosed. In some embodiments, the radioactive substance comprise, but are not limited to, ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ¹⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. In some embodiments, the anti-Gal3 antibody conjugated to a radioactive substance is used to detect and/or diagnose a subject as having, or being at risk of having, one or more neurological disorders. In some embodiments, the anti-Gal3 antibody conjugated to ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac, or another radioactive substance is used to detect and/or diagnose a subject as having, or being at risk of having, a neurological disorder having a physical cause, such as external or internal mechanical trauma (e.g. stroke or concussion), biological trauma (e.g. infection), chemical trauma (e.g. toxins or drugs), aging and age-related senescence, genetics, and many other causes. In some embodiments, the neurological disorder is caused by the effect or accumulation of mutated or misfolded proteins. In some embodiments, the anti-Gal3 antibody conjugated to a radioactive substance is used to detect and/or diagnose a subject as having, or being at risk of having, inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer, or otherwise known by a person skilled in the art. In some embodiments, the anti-Gal3 antibody conjugated to a radioactive substance is used to detect and/or diagnose a subject as having, or being at risk of having, one or more neurological disorders categorized as proteopathies. In some embodiments, the anti-Gal3 antibody conjugated to ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac, or another radioactive substance is used to detect and/or diagnose a subject as having, or being at risk of having, inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer, or otherwise known by a person skilled in the art.

In some embodiments, the anti-Gal3 antibody conjugated to a radioactive substance is used to detect and/or diagnose a subject as having, or being at risk of having, one or more proteopathies. In some embodiments, the anti-Gal3 antibody conjugated to ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ³⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸ Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac, or another radioactive substance is used to detect and/or diagnose a subject as having, or being at risk of having, one or more proteopathies. In some embodiments, the anti-Gal3 antibody conjugated to a radioactive substance is used to detect and/or diagnose a subject as having, or being at risk of having, Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Friedreich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or otherwise known by a person skilled in the art, or any combination thereof. In some embodiments, the anti-Gal3 antibody conjugated to ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au. ¹⁹⁹Au, ²¹¹At, ²¹¹Pb, ²¹²Bi, ²¹²Pb, ²¹³Bi, ²²³Ra and ²²⁵Ac, or another radioactive substance is used to detect and/or diagnose a subject as having, or being at risk of having Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Friedreich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or otherwise known by a person skilled in the art, or any combination thereof.

In some embodiments, the methods disclosed herein further comprise detecting a detectable marker in the subject. In some embodiments, the detectable marker is detected in the brain, heart, lungs, kidney, bones, plasma, other body systems, and/or any combination thereof. In some embodiments, the detectable marker comprises a radioactive substance. In some embodiments, the radioactive substance is detected in the subject's brain, heart, lungs, kidney, bones, plasma, blood, and other body systems. In some embodiments, the radioactive substance is detected using one or more imaging devices. For example, in some embodiments, the detectable marker comprises a radioactive substance that is detected by gamma camera, SPECT, PET, MRI, CT, or any combination thereof. In some embodiments, the detection device is chosen for its suitability in detecting a given detectable marker. It is contemplated that any suitable imaging or detection method would be suitable for use with the methods disclosed herein. In some embodiments, the anti-Gal3 antibody conjugated to a detectable marker is used as a primary means of diagnosis or risk assessment. In some embodiments, the anti-Gal3 antibody conjugated to a detectable marker is used as a supplement to one or more additional methods of diagnosis and/or risk assessment.

In some embodiments, diagnosing a subject as having one or more diseases comprises an assessment of Gal3 detection in the subject and in a healthy subject. In some embodiments, some embodiments, the Gal3 detection comprises administering an anti-Gal3 antibody that is conjugated to a detectable moiety to a healthy subject and detecting the detectable moiety. In some embodiments, the detectable moiety comprises a radioactive substance. In some embodiments, the diagnosis of one or more diseases comprises an assessment of Gal3 levels in a subject having, at risk of having, or suspected of having or being at risk of having, one or more diseases. In some embodiments, detectable Gal3 levels in a subject having or at risk of having one or more diseases is indicated by a subject having about 1, 2, 3, 4, 5, 6, 7, 8, 9, 10, 15, 20, 25, 30, 40, 50, 60, 70, 75, 80, 90, 100, 125, 150, 175, 200, 250, 300, 400, 500, 600, 750, 800, 900, or 1000%, more detectable Gal3 than a health subject, or having a detectable Gal3 level that is in a range defined by any two of the preceding values. For example, in some embodiments, detectable Gal3 levels in a subject having or at risk of having one or more diseases is indicated by a subject having between about 1-1000, 1-750, 1-500, 1-250, 1-100, 1-75, 1-50, 1-25, 1-10, −15, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000%, more detectable Gal3 than a healthy subject.

In some embodiments, the diagnosis of a subject as having one or more diseases comprises an assessment of Gal3 levels in a subject having, at risk of having, or suspected of having or being at risk of having, a neurological disorder having a physical cause, such as external or internal mechanical trauma (e.g. stroke or concussion), biological trauma (e.g. infection), chemical trauma (e.g. toxins or drugs), aging and age-related senescence, genetics, and many other causes. In some embodiments, the anti-Gal3 antibody conjugated to a radioactive substance is used to detect and/or diagnose a subject as having, or being at risk of having, one or more neurological disorders. In some embodiments, the anti-Gal3 antibody conjugated to ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I. ¹⁴²Pr. ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Tr, ¹⁹⁸Au, ¹⁹⁹Au, ²¹¹At, ²¹¹Pb, ²¹²Bi, ²¹²Pb, ²¹³Bi, ²²³Ra and ²²⁵Ac, or another radioactive substance is used to detect and/or diagnose a subject as having, or being at risk of having, inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer, or otherwise known by a person skilled in the art. In some embodiments, detectable Gal3 levels in a subject having or at risk of having inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer, or otherwise known by a person skilled in the art is indicated by a subject having about 1, 2, 3, 4, 5, 6, 7, 8, 9, 10, 15, 20, 25, 30, 40, 50, 60, 70, 75, 80, 90, 100, 125, 150, 175, 200, 250, 300, 400, 500, 600, 750, 800, 900, or 1000%, more detectable Gal3 than a health subject, or having a detectable Gal3 level that is in a range defined by any two of the preceding values. For example, in some embodiments, detectable Gal3 levels in a subject having or at risk of having inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer, or otherwise known by a person skilled in the artis indicated by a subject having between about 1-1000, 1-750, 1-500, 1-250, 1-100, 1-75, 1-50, 1-25, 1-10, −15, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000%, more detectable Gal3 than a health subject.

In some embodiments, the anti-Gal3 antibody conjugated to a radioactive substance is used to detect and/or diagnose a subject as having, or being at risk of having, one or more neurological disorders. In some embodiments, the anti-Gal3 antibody conjugated to ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac, or another radioactive substance is used to detect and/or diagnose a subject as having, or being at risk of having, Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Friedreich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or otherwise known by a person skilled in the art, or any combination thereof. In some embodiments, detectable Gal3 levels in a subject having or at risk of having Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Friedreich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or otherwise known by a person skilled in the art, or any combination thereof, is indicated by a subject having about 1, 2, 3, 4, 5, 6, 7, 8, 9, 10, 15, 20, 25, 30, 40, 50, 60, 70, 75, 80, 90, 100, 125, 150, 175, 200, 250, 300, 400, 500, 600, 750, 800, 900, or 1000%, more detectable Gal3 than a health subject, or having a detectable Gal3 level that is in a range defined by any two of the preceding values. For example, in some embodiments, detectable Gal3 levels in a subject having or at risk of having, Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Friedreich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or otherwise known by a person skilled in the art, or any combination thereof, is indicated by a subject having between about 1-1000, 1-750, 1-500, 1-250, 1-100, 1-75, 1-50, 1-25, 1-10, −15, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000%, more detectable Gal3 than a health subject.

In some embodiments, the anti-Gal3 antibody conjugated to a radioactive substance is used to detect and/or diagnose a subject as having, or being at risk of having, one or more diseases. In some embodiments, the anti-Gal3 antibody conjugated to ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁸Re. ¹⁸⁹Re, ¹⁹⁴Ir, ¹⁸⁶Re, ¹⁹⁸Au, ¹⁹⁹Au, ²¹¹At, ²¹¹Pb, ²¹²Bi, ²¹²Pb, ²¹³Bi, ²²³Ra and ²²⁵Ac, or another radioactive substance is used to detect and/or diagnose a subject as having, or being at risk of having one or more diseases. In some embodiments, diagnosing a subject as having one or more diseases comprises an assessment of Gal3 levels in the subject's brain and in the brain of a healthy subject. In some embodiments, the Gal3 detection comprises administering an anti-Gal3 antibody that is conjugated to a detectable moiety to a healthy subject and detecting or failing to detect the detectable moiety in the healthy subject's brain. In some embodiments, the detectable moiety comprises a radioactive substance. In some embodiments, the diagnosis of one or more diseases comprises an assessment of Gal3 levels in a subject having, at risk of having, or suspected of having or being at risk of having, one or more diseases. In some embodiments, detectable Gal3 levels in a subject having or at risk of having one or more diseases is indicated by a subject having about 1, 2, 3, 4, 5, 6, 7, 8, 9, 10, 15, 20, 25, 30, 40, 50, 60, 70, 75, 80, 90, 100, 125, 150, 175, 200, 250, 300, 400, 500, 600, 750, 800, 900, or 1000%, more detectable Gal3 in their brain than a health subject, or having a detectable Gal3 level in their brain that is in a range defined by any two of the preceding values. For example, in some embodiments, detectable Gal3 levels in a subject having or at risk of having one or more diseases is indicated by a subject having between about 1-1000, 1-750, 1-500, 1-250, 1-100, 1-75, 1-50, 1-25, 1-10, −15, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000%, more detectable Gal3 in their brain than a health subject.

In some embodiments, the anti-Gal3 antibody conjugated to a radioactive substance is used to detect and/or diagnose a subject as having, or being at risk of having, one or more diseases. In some embodiments, the anti-Gal3 antibody conjugated to ¹⁸F, ¹⁸F-FAC, ³²p, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac, or another radioactive substance is used to detect and/or diagnose a subject as having, or being at risk of having, one or more diseases. In some embodiments, diagnosing a subject as having one or more diseases comprises an assessment of Gal3 levels in the subject's heart and in the heart of a healthy subject. In some embodiments, the Gal3 detection comprises administering an anti-Gal3 antibody that is conjugated to a detectable moiety to a healthy subject and detecting or failing to detect the detectable moiety in the healthy subject's heart. In some embodiments, the detectable moiety comprises a radioactive substance. In some embodiments, the diagnosis of one or more diseases comprises an assessment of Gal3 levels in a subject having, at risk of having, or suspected of having or being at risk of having, one or more diseases. In some embodiments, detectable Gal3 levels in a subject having or at risk of having one or more diseases is indicated by a subject having about 1, 2, 3, 4, 5, 6, 7, 8, 9, 10, 15, 20, 25, 30, 40, 50, 60, 70, 75, 80, 90, 100, 125, 150, 175, 200, 250, 300, 400, 500, 600, 750, 800, 900, or 1000%, more detectable Gal3 in their heart than a health subject, or having a detectable Gal3 level in their heart that is in a range defined by any two of the preceding values. For example, in some embodiments, detectable Gal3 levels in a subject having or at risk of having one or more diseases is indicated by a subject having between about 1-1000, 1-750, 1-500, 1-250, 1-100, 1-75, 1-50, 1-25, 1-10, −15, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000%, more detectable Gal3 in their heart than a healthy subject.

In some embodiments, the anti-Gal3 antibody conjugated to a radioactive substance is used to detect and/or diagnose a subject as having, or being at risk of having, one or more diseases. In some embodiments, the anti-Gal3 antibody conjugated to ¹⁸F, ¹⁸F-FAC, ³²p, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ¹⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm. ¹⁵⁴⁻¹⁵⁸ Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac, or another radioactive substance is used to detect and/or diagnose a subject as having, or being at risk of having, one or more diseases. In some embodiments, diagnosing a subject as having one or more diseases comprises an assessment of Gal3 levels in the subject's plasma and in the plasma of a healthy subject. In some embodiments, the Gal3 detection comprises administering an anti-Gal3 antibody that is conjugated to a detectable moiety to a healthy subject and detecting or failing to detect the detectable moiety in the healthy subject's plasma. In some embodiments, the detectable moiety comprises a radioactive substance. In some embodiments, the diagnosis of one or more diseases comprises an assessment of Gal3 levels in a subject having, at risk of having, or suspected of having or being at risk of having, one or more diseases. In some embodiments, detectable Gal3 levels in a subject having or at risk of having one or more diseases is indicated by a subject having about 1, 2, 3, 4, 5, 6, 7, 8, 9, 10, 15, 20, 25, 30, 40, 50, 60, 70, 75, 80, 90, 100, 125, 150, 175, 200, 250, 300, 400, 500, 600, 750, 800, 900, or 1000%, more detectable Gal3 in their plasma than a health subject, or having a detectable Gal3 level in their plasma that is in a range defined by any two of the preceding values. For example, in some embodiments, detectable Gal3 levels in a subject having or at risk of having one or more diseases is indicated by a subject having between about 1-1000, 1-750, 1-500, 1-250, 1-100, 1-75, 1-50, 1-25, 1-10, −15, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000%, more detectable Gal3 in their plasma than a healthy subject.

In some embodiments, the anti-Gal3 antibody conjugated to a radioactive substance is used to detect and/or diagnose a subject as having, or being at risk of having, one or more diseases. In some embodiments, the anti-Gal3 antibody conjugated to ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸ Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re. ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac, or another radioactive substance is used to detect and/or diagnose a subject as having, or being at risk of having, one or more diseases. In some embodiments, diagnosing a subject as having one or more diseases comprises an assessment of Gal3 levels in the subject's blood and in the blood of a healthy subject. In some embodiments, the Gal3 detection comprises administering an anti-Gal3 antibody that is conjugated to a detectable moiety to a healthy subject and detecting or failing to detect the detectable moiety in the healthy subject's plasma. In some embodiments, the detectable moiety comprises a radioactive substance. In some embodiments, the diagnosis of one or more diseases comprises an assessment of Gal3 levels in a subject having, at risk of having, or suspected of having or being at risk of having, one or more diseases. In some embodiments, detectable Gal3 levels in a subject having or at risk of having one or more diseases is indicated by a subject having about 1, 2, 3, 4, 5, 6, 7, 8, 9, 10, 15, 20, 25, 30, 40, 50, 60, 70, 75, 80, 90, 100, 125, 150, 175, 200, 250, 300, 400, 500, 600, 750, 800, 900, or 1000%, more detectable Gal3 in their blood than a health subject, or having a detectable Gal3 level in their plasma that is in a range defined by any two of the preceding values. For example, in some embodiments, detectable Gal3 levels in a subject having or at risk of having one or more diseases is indicated by a subject having between about 1-1000, 1-750, 1-500, 1-250, 1-100, 1-75, 1-50, 1-25, 1-10,-15, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000%, more detectable Gal3 in their blood than a healthy subject.

In some embodiments, the anti-Gal3 antibody conjugated to a radioactive substance is used to detect and/or diagnose a subject as having, or being at risk of having, one or more diseases. In some embodiments, the anti-Gal3 antibody conjugated to ¹⁸F, ¹⁸F-FAC, ³²p, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac, or another radioactive substance is used to detect and/or diagnose a subject as having, or being at risk of having, one or more diseases. In some embodiments, diagnosing a subject as having one or more diseases comprises an assessment of Gal3 levels in one or both of the subject's lungs and in one or both of the lungs of a healthy subject. In some embodiments, the Gal3 detection comprises administering an anti-Gal3 antibody that is conjugated to a detectable moiety to a healthy subject and detecting or failing to detect the detectable moiety in one or both of the healthy subject's lungs. In some embodiments, the detectable moiety comprises a radioactive substance. In some embodiments, the diagnosis of one or more diseases comprises an assessment of Gal3 levels in a subject having, at risk of having, or suspected of having or being at risk of having, one or more diseases. In some embodiments, detectable Gal3 levels in a subject having or at risk of having one or more diseases is indicated by a subject having about 1, 2, 3, 4, 5, 6, 7, 8, 9, 10, 15, 20, 25, 30, 40, 50, 60, 70, 75, 80, 90, 100, 125, 150, 175, 200, 250, 300, 400, 500, 600, 750, 800, 900, or 1000%, more detectable Gal3 in one or both of their lungs than a healthy subject, or having a detectable Gal3 level in one or both of their lungs that is in a range defined by any two of the preceding values. For example, in some embodiments, detectable Gal3 levels in a subject having or at risk of having one or more diseases is indicated by a subject having between about 1-1000, 1-750, 1-500, 1-250, 1-100, 1-75, 1-50, 1-25, 1-10, −15, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000%, more detectable Gal3 in one or both of their lungs than a healthy subject.

In some embodiments, the anti-Gal3 antibody conjugated to a radioactive substance is used to detect and/or diagnose a subject as having, or being at risk of having, one or more diseases. In some embodiments, the anti-Gal3 antibody conjugated to ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re. ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac, or another radioactive substance is used to detect and/or diagnose a subject as having, or being at risk of having, one or more diseases. In some embodiments, diagnosing a subject as having one or more diseases comprises an assessment of Gal3 levels in one or both of the subject's kidneys and in one or both of the kidneys of a healthy subject. In some embodiments, the Gal3 detection comprises administering an anti-Gal3 antibody that is conjugated to a detectable moiety to a healthy subject and detecting or failing to detect the detectable moiety in one or both of the healthy subject's kidneys. In some embodiments, the detectable moiety comprises a radioactive substance. In some embodiments, the diagnosis of one or more diseases comprises an assessment of Gal3 levels in a subject having, at risk of having, or suspected of having or being at risk of having, one or more diseases. In some embodiments, detectable Gal3 levels in a subject having or at risk of having one or more diseases is indicated by a subject having about 1, 2, 3, 4, 5, 6, 7, 8, 9, 10, 15, 20, 25, 30, 40, 50, 60, 70, 75, 80, 90, 100, 125, 150, 175, 200, 250, 300, 400, 500, 600, 750, 800, 900, or 1000%, more detectable Gal3 in one or both of their kidneys than a healthy subject, or having a detectable Gal3 level in one or both of their kidneys that is in a range defined by any two of the preceding values. For example, in some embodiments, detectable Gal3 levels in a subject having or at risk of having one or more diseases is indicated by a subject having between about 1-1000, 1-750, 1-500, 1-250, 1-100, 1-75, 1-50, 1-25, 1-10, −15, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000%, more detectable Gal3 in one or both of their kidneys than a healthy subject.

In some embodiments, the anti-Gal3 antibody conjugated to a radioactive substance is used to detect and/or diagnose a subject as having, or being at risk of having, one or more diseases. In some embodiments, the anti-Gal3 antibody conjugated to ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸ Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac, or another radioactive substance is used to detect and/or diagnose a subject as having, or being at risk of having, one or more diseases. In some embodiments, diagnosing a subject as having one or more diseases comprises an assessment of Gal3 levels in one or more of the subject's bones and in one or more of the bones of a healthy subject. In some embodiments, the Gal3 detection comprises administering an anti-Gal3 antibody that is conjugated to a detectable moiety to a healthy subject and detecting or failing to detect the detectable moiety in one or more of the healthy subject's bones. In some embodiments, the detectable moiety comprises a radioactive substance. In some embodiments, the diagnosis of one or more diseases comprises an assessment of Gal3 levels in a subject having, at risk of having, or suspected of having or being at risk of having, one or more diseases. In some embodiments, detectable Gal3 levels in a subject having or at risk of having one or more diseases is indicated by a subject having about 1, 2, 3, 4, 5, 6, 7, 8, 9, 10, 15, 20, 25, 30, 40, 50, 60, 70, 75, 80, 90, 100, 125, 150, 175, 200, 250, 300, 400, 500, 600, 750, 800, 900, or 1000%, more detectable Gal3 in one or more of their bones than a healthy subject, or having a detectable Gal3 level in their heart that is in a range defined by any two of the preceding values. For example, in some embodiments, detectable Gal3 levels in a subject having or at risk of having one or more diseases is indicated by a subject having between about 1-1000, 1-750, 1-500, 1-250, 1-100, 1-75, 1-50, 1-25, 1-10, −15, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000%, more detectable Gal3 in one or more of their bones than a healthy subject.

### Kit/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include an anti-Gal3 antibody as disclosed herein, host cells for producing one or more antibodies described herein, and/or vectors comprising nucleic acid molecules that encode the antibodies described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### NUMBERED ARRANGEMENTS

Some embodiments provided herein are described by way of the following provided numbered arrangements and also provided as possible combinations or overlapping embodiments:
1. A method of increasing a subject's Mini Mental State Examination (MMSE) score, the method comprising:
   identifying a subject as likely to benefit from an increased MMSE score, and
   administering a therapeutically effective amount of an antibody, wherein the antibody binds to Gal3.
2. A method of increasing a subject's Mini Mental State Examination (MMSE) score, the method comprising:
   identifying a subject as having Alzheimer's disease (AD), and
   administering a therapeutically effective amount of an antibody, wherein the antibody binds to Gal3.
3. A method of increasing a subject's Mini Mental State Examination (MMSE) score, the method comprising:
   identifying a subject as having dementia, and
   administering a therapeutically effective amount of an antibody, wherein the antibody binds to Gal3.
4. A method of increasing a subject's Mini Mental State Examination (MMSE) score, the method comprising:
   identifying a subject as likely to benefit from an increased MMSE score, and
   administering 420 mg of an anti-Gal3 antibody, wherein
   the antibody is administered once weekly for 5 weeks.
5. A method of increasing a subject's Mini Mental State Examination (MMSE) score, the method comprising:
   identifying a subject as likely to benefit from an increased MMSE score, and
   administering 1000 mg of an anti-Gal3 antibody, wherein
   the antibody is administered once weekly for 5 weeks.
6. A method of increasing a subject's Mini Mental State Examination (MMSE) score, the method comprising:
   identifying a subject as likely to benefit from an increased MMSE score, and
   administering 140 mg of an anti-Gal3 antibody once weekly for 5 weeks followed by administration of 4000 mg of the antibody once monthly for one or more months.
7. A method of increasing a subject's Mini Mental State Examination (MMSE) score, the method comprising:
   identifying a subject as likely to benefit from an increased MMSE score, and
   administering 420 mg of an anti-Gal3 antibody once weekly for 5 weeks followed by administration of 4000 mg of the antibody once monthly for one or more months.
8. A method of increasing a subject's Mini Mental State Examination (MMSE) score, the method comprising:
   identifying a subject as likely to benefit from an increased MMSE score, and
   administering 1000 mg of an anti-Gal3 antibody once weekly for 5 weeks followed by administration of 4000 mg of the antibody once monthly for one or more months.
9. The method of any one of the preceding arrangements, wherein
   administration of 1000 mg of an anti-Gal3 antibody once weekly for 5 weeks followed by administration of 4000 mg of the antibody once monthly for one or more months increases the subject's MMSE score by up to about 11 points.
10. A method of increasing a subject's Mini Mental State Examination (MMSE) score, the method comprising:
   identifying a subject as likely to benefit from an increased MMSE score, and
   administering 1000 mg of an anti-Gal3 antibody once weekly for 5 weeks followed by administration of 4000 mg of the antibody once monthly for one or more months, and wherein
   administration of the antibody increases the subject's MMSE score by up to about 11 points.
11. A method of increasing a subject's EQ-5D-5L score, the method comprising:
   identifying a subject as likely to benefit from an increased EQ-5D-5L score, and
   administering a therapeutically effective amount of an antibody, wherein the antibody binds to Gal3.
12. A method of increasing a subject's a subject's EuroQol 5-Dimension 5-Level (EQ-5D-5L) score, the method comprising:
   identifying a subject as having Alzheimer's disease (AD), and
   administering a therapeutically effective amount of an antibody, wherein the antibody binds to Gal3.
13. A method of increasing a subject's a subject's EuroQol 5-Dimension 5-Level (EQ-5D-5L) score, the method comprising:
   identifying a subject as having dementia, and
   administering a therapeutically effective amount of an antibody, wherein the antibody binds to Gal3.
14. A method of decreasing a subject's Neuropsychiatric Inventory (NPI) score, the method comprising:
   identifying a subject as likely to benefit from a decreased Neuropsychiatric Inventory (NPI) score, and
   administering a therapeutically effective amount of an antibody, wherein the antibody binds to Gal3.
15. A method of decreasing a subject's a subject's Neuropsychiatric Inventory (NPI) score, the method comprising:
   identifying a subject as having Alzheimer's disease (AD), and
   administering a therapeutically effective amount of an antibody, wherein the antibody binds to Gal3.
16. A method of decreasing a subject's a subject's Neuropsychiatric Inventory (NPI) score, the method comprising:
   identifying a subject as having dementia, and
   administering a therapeutically effective amount of an antibody, wherein the antibody binds to Gal3.
17. A method of decreasing a subject's Clinical Dementia Rating Scale-Sum of Boxes (CDR-SB), the method comprising:
   identifying a subject as likely to benefit from a decreased CDR-SB score, and
   administering a therapeutically effective amount of an antibody, wherein the antibody binds to Gal3.
18. A method of decreasing a subject's Clinical Dementia Rating Scale-Sum of Boxes (CDR-SB) score, the method comprising:
   identifying a subject as having Alzheimer's disease (AD), and
   Administering an antibody, wherein the antibody binds to Gal3.
19. A method of decreasing a subject's Clinical Dementia Rating Scale-Sum of Boxes (CDR-SB) score, the method comprising:
   identifying a subject as having dementia, and
   administering an antibody, wherein the antibody binds to Gal3.
20. A method of decreasing a subject's Clinical Dementia Rating Scale-Sum of Boxes (CDR-SB) score, the method comprising:
   identifying a subject as likely to benefit from a decreased CDR-SB score, and
   administering 140 mg of an anti-Gal3 antibody, wherein
   the antibody is administered once weekly for 5 weeks.
21. A method of decreasing a subject's Clinical Dementia Rating Scale-Sum of Boxes (CDR-SB) score, the method comprising:
   identifying a subject as likely to benefit from a decreased CDR-SB score, and
   administering 420 mg of an anti-Gal3 antibody, wherein
   the antibody is administered once weekly for 5 weeks.
22. A method of decreasing a subject's Clinical Dementia Rating Scale-Sum of Boxes (CDR-SB) score, the method comprising:
   identifying a subject as likely to benefit from a decreased CDR-SB score, and
   administering 1000 mg of an anti-Gal3 antibody, wherein
   the antibody is administered once weekly for 5 weeks.
23. A method of decreasing a subject's Clinical Dementia Rating Scale-Sum of Boxes (CDR-SB) score, the method comprising:
   identifying a subject as likely to benefit from a decreased CDR-SB score, and
   administering an anti-Gal3 antibody, wherein
   140 mg of the antibody is administered once weekly for 5 weeks followed by administration of 4000 mg of the antibody once monthly for one or more months.
24. A method of decreasing a subject's Clinical Dementia Rating Scale-Sum of Boxes (CDR-SB) score, the method comprising:
   identifying a subject as likely to benefit from a decreased CDR-SB score, and
   administering an anti-Gal3 antibody, wherein
   420 mg of the antibody is administered once weekly for 5 weeks followed by administration of 4000 mg of the antibody once monthly for one or more months.
25. A method of decreasing a subject's Clinical Dementia Rating Scale-Sum of Boxes (CDR-SB) score, the method comprising:
   identifying a subject as likely to benefit from a decreased CDR-SB score, and
   administering an anti-Gal3 antibody, wherein
   1000 mg of the antibody is administered once weekly for 5 weeks followed by administration of 4000 mg of the antibody once monthly for one or more months.
26. The method of any one of the preceding arrangements, wherein
   administration of 1000 mg of an anti-Gal3 antibody once weekly for 5 weeks followed by administration of 4000 mg of the antibody once monthly for one or more months decreases the subject's Clinical Dementia Rating Scale-Sum of Boxes (CDR-SB) score by up to about 0.5 points.
27. A method of decreasing a subject's Clinical Dementia Rating Scale-Sum of Boxes (CDR-SB) score, the method comprising:
   identifying a subject as likely to benefit from a decreased CDR-SB score, and
   administering an anti-Gal3 antibody, wherein
   1000 mg of the antibody is administered once weekly for 5 weeks followed by administration of 4000 mg of the antibody once monthly for one or more months, and wherein
   administration of the antibody decreases the subject's Clinical Dementia Rating Scale-Sum of Boxes (CDR-SB) score by up to about 0.5 points.
28. A method of increasing a subject's Cognitive Drug Research battery score, the method comprising:
   identifying a subject as likely to benefit from an increased Cognitive Drug Research battery score, and
   administering a therapeutically effective amount of an antibody, wherein the antibody binds to Gal3.
29. A method of increasing a subject's Cognitive Drug Research battery score, the method comprising:
   identifying a subject as having Alzheimer's disease (AD), and
   administering a therapeutically effective amount of an antibody, wherein the antibody binds to Gal3.
30. A method of increasing a subject's Cognitive Drug Research battery score, the method comprising:
   identifying a subject as having dementia, and
   administering a therapeutically effective amount of an antibody, wherein the antibody binds to Gal3.
31. A method of increasing a subject's strength, locomotor functions, and/or balance, the method comprising:
   identifying a subject as likely to benefit from increased strength, locomotor functions, and/or balance, and
   administering a therapeutically effective amount of an antibody, wherein the antibody binds Gal3.
32. A method of increasing a subject's strength, locomotor functions, and/or balance, the method comprising:
   identifying a subject as having Alzheimer's disease (AD), and
   administering a therapeutically effective amount of an antibody, wherein the antibody binds Gal3.
33. A method of increasing a subject's strength, locomotor functions, and/or balance, the method comprising:
   identifying a subject as having dementia, and
   administering a therapeutically effective amount of an antibody, wherein the antibody binds Gal3.
34. A method of decreasing the frequency and/or severity of symptoms related to aging and/or aging related senescence in a subject in need thereof, the method comprising:
   identifying a subject as likely to benefit from decreased frequency and/or severity of symptoms related to aging and/or aging related senescence, and
   administering a therapeutically effective amount of an antibody, wherein the antibody binds Gal3.
35. A method of increasing a subject's whole brain volume, the method comprising:
   identifying a subject as likely to benefit from an increased whole brain volume, and
   administering a therapeutically effective amount of an antibody, wherein the antibody binds to Gal3.
36. A method of decreasing brain atrophy in a subject, the method comprising:
   identifying a subject as likely to benefit from decreased brain atrophy, and
   administering a therapeutically effective amount of an antibody, wherein the antibody binds to Gal3.
37. A method of increasing a subject's performance on one or more standard cognitive assessments, the method comprising:
   identifying a subject as likely to benefit from increased performance on the one or more standard cognitive assessments, and
   administering a therapeutically effective amount of an antibody, wherein the antibody binds to Gal3.
38. A method of treating a patient, the method comprising:
   identifying a patient, wherein
      the patient is identified as having a risk of a decreased MMSE score, decreased EQ-5D-5L score, increased CDR-SB score, decreased NPI score, decreased Cognitive Drug Research battery, increased brain atrophy, decreased whole brain volume, increased symptoms related to aging and/or aging related senescence, decreased cognitive ability, decreased strength, decreased locomotor functions, decreased balance, or any combination thereof, and administering a therapeutically effective amount of an anti Gal3 Ab to the patient to reduce said risk.
39. The method of any of the preceding arrangements, wherein the one or more cognitive assessments comprise a MMSE, CDR-SB, EQ-5D-5L, NPI, a Cognitive Drug Research battery, or any combination thereof.
40. The method of any one of the preceding arrangements, further comprising performing a MMSE, CDR-SB, EQ-5D-5L, NPI, Cognitive Drug Research battery, or any combination thereof, on the subject prior to administration of the antibody.
41. The method of any one of the preceding arrangements, further comprising performing a MMSE, CDR-SB, EQ-5D-5L, NPI, Cognitive Drug Research battery, or any combination thereof, on the subject up to 1, 5, 10, 15, 28, 29, 30, 31, 36, 40, 45, 50, 55, 60, 64, 65, 70, 75, 80, 85, 90, 95, 100, 104, 105, 110, 120, 130, 140, 150, 160, 170, or 180 days after administration of the antibody, or at intervals that are defined by any two of the preceding values.
42. The method of any one of the preceding arrangements, further comprising performing a MMSE, CDR-SB, EQ-5D-5L, NPI, Cognitive Drug Research battery, or any combination thereof, on the subject at least once every 1, 2, 3, 4, 5, 6, 7, 8, 9, 10, 11, or 12 months after administration of the antibody, or at intervals that are defined by any two of the preceding values.
43. The method of any one of the preceding arrangements, wherein the subject has Alzheimer's disease.
44. The method of any one of the preceding arrangements, wherein the subject has, or has had, inflammation, an autoimmune disorder, diabetes, cardiovascular disease, organ dysfunction, organ damage, kidney dysfunction, kidney damage, liver dysfunction, liver damage, heart dysfunction, heart damage, heart failure, a bone disorder, anemia, a hematopoietic disorder, a metabolic disease, cancer, an immune deficiency, neurodegenerative disease, Parkinson's disease, Alzheimer's disease, fibrosis, frailty, respiratory system disease or disorder, or any combination thereof.
45. The method of any one of the preceding arrangements, further comprising performing one or more PET scans on the subject.
46. The method of any one of the preceding arrangements, further comprising performing one or more MRI scans on the subject.
47. The method of any one of the preceding arrangements, further comprising monitoring one or more of the subject's biomarkers following antibody administration.
48. The method of any one of the preceding arrangements, wherein changes in the subject's score on the MMSE, CDR-SB, EQ-5D-5L, NPI, Cognitive Drug Research battery, or any combination thereof, is determined by comparing the subject's score on the MMSE, CDR-SB, EQ-5D-5L, NPI, or Cognitive Drug Research battery, prior to administration of the antibody to the subject's score at one or more timepoints following administration of the antibody.
49. The method of any one of the preceding arrangements wherein a subject's whole brain volume is measured by MRI.
50. The method of any one of the preceding arrangements, wherein the CDR-SB test comprises one or more tests of the subject's memory, orientation, judgement and problem solving, community affairs, home and hobbies, personal care, or any combination thereof.
51. The method of any one of the preceding arrangements, wherein the subject's CDR-SB score is decreased by at least 0.5, 1, 1.5, 2, 2.5, 3, 3.5, 4, 4.5, 5, 6, 7, 8, or nine points, or by a range that is defined by any two of the preceding values.
52. The method of any one of the preceding arrangements, wherein the subject's mean global CDR-SB score is decreased by at least 0.5, 1, 1.5, 2, 2.5, 3, 3.5, 4, 4.5, 5, 6, 7, 8, or 9 fold, or by a range that is defined by any two of the preceding values.
53. The method of any one of the preceding arrangements, wherein the subject's mean global CDR-SB score is decreased by at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 points.
54. The method of any one of the preceding arrangements, wherein the subject's score on tests of memory, orientation, judgement and problem solving, community affairs, home and hobbies, and/or personal care score on the CDR-SB, are decreased by at least 0.5, 1, 2, or 3 points, or by a range that is defined by any two of the preceding values.
55. The method of any one of the preceding arrangements, wherein the subject's mean Memory CDR-SB subdomain score is decreased by at least 0.5, 1, 1.5, 2, 2.5, 3, 3.5, 4, 4.5, 5, 6, 7, 8, or 9 fold, or by a range that is defined by any two of the preceding values.
56. The method of any one of the preceding arrangements, wherein the subject's mean Memory CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5 points.
57. The method of any one of the preceding arrangements, wherein the subject's mean Orientation CDR-SB subdomain score is decreased by at least 0.5, 1, 1.5, 2, 2.5, 3, 3.5, 4, 4.5, 5, 6, 7, 8, or 9 fold, or by a range that is defined by any two of the preceding values.
58. The method of any one of the preceding arrangements, wherein the subject's mean Orientation CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5 points.
59. The method of any one of the preceding arrangements, wherein the subject's mean Judgement and Problem Solving CDR-SB subdomain score is decreased by at least 0.5, 1, 1.5, 2, 2.5, 3, 3.5, 4, 4.5, 5, 6, 7, 8, or 9 fold, or by a range that is defined by any two of the preceding values.
60. The method of any one of the preceding arrangements, wherein the subject's mean Judgement and Problem Solving CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5 points.
61. The method of any one of the preceding arrangements, wherein the subject's mean Community Affairs CDR-SB subdomain score is decreased by at least 0.5, 1, 1.5, 2, 2.5, 3, 3.5, 4, 4.5, 5, 6, 7, 8, or 9 fold, or by a range that is defined by any two of the preceding values.
62. The method of any one of the preceding arrangements, wherein the subject's mean Community Affairs CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5 points.
63. The method of any one of the preceding arrangements, wherein the subject's mean Home and Hobbies CDR-SB subdomain score is decreased by at least 0.5, 1, 1.5, 2, 2.5, 3, 3.5, 4, 4.5, 5, 6, 7, 8, or 9 fold, or by a range that is defined by any two of the preceding values.
64. The method of any one of the preceding arrangements, wherein the subject's mean Home and Hobbies CDR-SB subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5 points.
65. The method of any one of the preceding arrangements, wherein the subject's mean Personal Care CDR-SB subdomain score is decreased by at least 0.5, 1, 1.5, 2, 2.5, 3, 3.5, 4, 4.5, 5, 6, 7, 8, or 9 fold, or by a range that is defined by any two of the preceding values.
66. The method of any one of the preceding arrangements, wherein the subject's mean Personal Care CDR-Subdomain score is decreased by at least 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.25, 0.26, 0.28, 0.30, 0.35, 0.40, 0.45, or 0.5 points.
67. The method of any one of the previous arrangements, wherein the subject's CDR-SB score is decreased by at least 5, 10, 15, 20, 25, 30, 40, 50, 60, 70, 75, 80, 90, 95, 96, 97, 98, 99%, or 100%.
68. The method of any one of the preceding arrangements, wherein the MMSE test comprises one or more tests of the subject's orientation to time, orientation to place, registration, attention and calculation, recall, naming, repetition, comprehension, reading, writing, and/or drawing.
69. The method of any one of the preceding arrangements, wherein the subject's MMSE score is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 points, or by a range that is defined by any two of the preceding values.
70. The method of any one of the preceding arrangements, wherein the subject's mean MMSE score is increased by at least 0.5, 1, 1.5, 2, 2.5, 3, 3.5, 4, 4.5, 5, 6, 7, 8, or 9 fold, or by a range that is defined by any two of the preceding values.
71. The method of any one of the preceding arrangements, wherein the MMSE test comprises one or more tests of the subject's orientation to time, orientation to place, registration, attention and calculation, recall, naming, repetition, comprehension, reading, writing, and/or drawing.
72. The method of any one of the preceding arrangements, wherein the subject's MMSE score is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 points, or by a range that is defined by any two of the preceding values.
73. The method of any one of the preceding arrangements, wherein the subject's mean MMSE score is increased by at least 0.5, 1, 1.5, 2, 2.5, 3, 3.5, 4, 4.5, 5, 6, 7, 8, or 9 fold, or by a range that is defined by any two of the preceding values.
74. The method of any one of the previous arrangements, wherein the subject's orientation to time, orientation to place, and/or attention and calculation score on the MMSE, are increased by at least 1, 2, 3, 4, or 5 points, or by a range that is defined by any two of the preceding values.
75. The method of any one of the preceding arrangements, wherein the subject's registration, recall, or comprehension score on the MMSE is increased by at least 1, 2, or 3 points, or by a range that is defined by any two of the preceding values.
76. The method of any one of the preceding arrangements, wherein the subject's repetition, reading, writing, or drawing score on the MMSE is increased by at least 1 point.
77. The method of any one of the preceding arrangements, wherein the MMSE score is increased by at least 5, 10, 15, 20, 25, 30, 40, 50, 60, 70, 75, 80, 90, 95, 96, 97, 98, 99, or 100%, or by a range that is defined by any two of the preceding values.
78. The method of any of the preceding arrangements, wherein the one or more biomarkers comprises a biomarker for inflammation, autoimmune disorders, diabetes, cardiovascular disease, organ dysfunction, organ damage, kidney dysfunction, kidney damage, liver dysfunction, liver damage, heart dysfunction, heart damage, heart failure, bone disorder, anemia, hematopoietic disorders, metabolic disease, hypercoagulable state, cancer, cancer progression, tumor formation, tumor progression, prothrombotic state in cancer and other acute phases, immune deficiency, morbidity, mortality, cell stress, DNA/protein damage, neurodegenerative disease, cellular senescence, cancer, Parkinson's disease, Alzheimer's disease, fibrosis, frailty, chronological age, biological age, physical capability, strength, locomotor function, balance, brain function, body composition, cardiovascular system, or respiratory system, and/or any combination of biomarkers thereof.
79. The method of any one of the preceding arrangements, wherein the one or more biomarkers comprise plasma Aβ40, Aβ42, tau (phosphorylated), NFL, NFH, and/or GAL-3, or any combination thereof.
80. The method of any one of the preceding arrangements, wherein the one or more biomarkers comprise Aβ42 levels.
81. The method of any one of the preceding arrangements, wherein the level of Aβ42 in a subject treated with TB006 is decreased by at least about 1, 2, 3, 4, 5, 6, 7, 8, 9, 10, 20, 25, 30, 40, 50, 60, 70, 75, 80, 90, 95, or 100%, as compared to the level of Aβ42 in the subject prior to treatment.
82. The method of any one of the preceding arrangements, wherein the level of Aβ42 in a subject treated with TB006 is decreased by at least about 0.5, 1, 1.5, 2, 2.5, 3, 3.5, 4, 4.5, 5, 6, 7, 8, or 9 fold, or by a range that is defined by any two of the preceding values as compared to the level of Aβ42 in the subject prior to treatment.
   The method of any one of the preceding arrangements, wherein the number of Aβ plaques in a subject treated with TB006 is decreased by at least about 1, 2, 3, 4, 5, 6, 7, 8, 9, 10, 20, 25, 30, 40, 50, 60, 70, 75, 80, 90, 95, or 100%, as compared to the number of Aβ plaques in the subject prior to treatment.
83. The method of any one of the preceding arrangements, wherein the number of Aβ plaques in a subject treated with TB006 is decreased by at least about 0.5, 1, 1.5, 2, 2.5, 3, 3.5, 4, 4.5, 5, 6, 7, 8, or 9 fold, or by a range that is defined by any two of the preceding values.
84. The method of any one of the preceding arrangements, further comprising measuring the subject's whole brain volume.
85. The method of any one of the preceding arrangements, further comprising measuring the subject's hippocampal brain volume.
86. The method of any one of the preceding arrangements, wherein hippocampal brain volume is measured by MRI.
87. The method of any one of the preceding arrangements, wherein hippocampal brain volume increases by at least about 0.01, 0.02, 0.03, 0.04, 0.05, 0.06, 0.07, 0.08, 0.09, 0.10, 0.12, 0.13, 0.14, 0.15, 0.16, 0.17, 0.18, 0.19, 0.20, 0.21, 0.22, 0.23, 0.24, or 0.25 mL.
88. The method of any one of the preceding arrangements, further comprising measuring the subject's intracranial brain volume.
89. The method of any one of the preceding arrangements, wherein hippocampal brain volume is increased in a subject treated with TB006 as compared to hippocampal brain volume in the subject prior to treatment.
90. The method of any one of the preceding arrangements, wherein intracranial brain volume is decreased in a subject treated with TB006 as compared to intracranial brain volume in the subject prior to treatment.
91. The method of any of the preceding arrangements, wherein the one or more morbidity, biomarkers comprise telomere length (TL), average TL, TL structure, shortest TL, DNA damage, Reactive Oxygen Species (ROS), mitochondrial dysfunction, autophagy, telomerase activity, gut microbiome, α-Klotho, adiponectin, sirtuin 1 (SIRT1), growth differentiation factor 15 (GDF15), sirtuin 6 (SIRT6), growth differentiation factor 11 (GDF11), skin microbiome, microRNA (miRNA), extracellular RNA (exRNA), grip strength, walking speed, standing balance, timed up and go test, atherosclerotic lesions, muscle mass, systolic blood pressure, cognitive function, body mass index, bone density, lung function, waist circumference, health assessments, or any combination thereof.
92. The method of any of the preceding arrangements, wherein the one or more inflammation biomarkers comprise lymphocytes, white blood cells, C-reactive protein (CRP/hsCRP), interleukins, IL6, tumor necrosis factor alpha (TNFα), monocytes, IL8, IL15, IL1-β, transforming growth factor beta (TGF-β), SIRT1, CXCL1, or any combination thereof.
93. The method of any of the preceding arrangements, wherein the one or more diabetes biomarkers comprise insulin, glucose, fasted glucose, glycated hemoglobin (Hba1c), C-peptide, adiponectin, SIRT 6, or any combination thereof.
94. The method of any one of the preceding arrangements, wherein the one or more cancer biomarkers comprise TNFα, extracellular vesicles (EVs), autophagy, TGF-β, SIRT1, plasminogen activator inhibitor-1 (PAI1), CXCL1, or any combination thereof.
95. The method of any one of the preceding arrangements, wherein the one or more cardiovascular disease biomarkers comprise CRP, hsCRP, cholesterol, LDL-cholesterol, triglycerides, HDL-cholesterol, atherosclerosis, atherosclerotic legions, blood pressure, systolic blood pressure, or any combination thereof.
96. The method of any one of the preceding arrangements, wherein the one or more kidney damage or dysfunction biomarkers comprise albumin, creatinine, GDF15, cystatin c, urea, or any combination thereof.
97. The method of any one of the preceding arrangements, wherein the one or more liver damage and/or disfunction biomarkers comprise albumin, alkaline phosphatase, bilirubin, adiponectin, GDF15, or any combination thereof.
98. The method of any one of the preceding arrangements, wherein the one or more anemia biomarkers comprise hemoglobin, hematocrit, RBC, mean corpuscular volume (MCV), mean corpuscular hemoglobin concentration (MCHC), or any combination thereof.
99. The method of any one of the preceding arrangements wherein the one or more physical capability biomarkers comprise grip strength, walking speed, standing balance, a timed up and go test, or any combination thereof.
100. The method of any one of the preceding arrangements, wherein the one or more organ function biomarkers comprise atherosclerotic lesions, muscle mass, systolic blood pressure, cognitive function, body mass index, bone density, lung function, waist circumference, or any combination thereof.
101. The method of any one of the preceding arrangements, wherein the one or more biomarkers are measured within at least 1, 2, 3, 4, 5, 6, 7, 8, 9, 10, 11, 12, 13, or 14 days after antibody administration, or at an interval that is defined by any two of the preceding values.
102. The method of any one of the preceding arrangements, wherein the antibody is at a dose of at least 70, 75, 80, 90, 100, 110, 120, 125, 130, 140, 150, 160, 170, 180, 190, 200, 250, 300, 350, 360, 370, 380, 390, 400, 410, 420, 430, 440, 450, 500, 600, 700, 800, 900, 950, 960, 970, 980, 990, 1000, 1010, 1020, 1025, 1030, 1040, 1050, 1100, 1200, 1300, 1400, 1500, 2000, 2100, 2200, 2300, 2400, 2500, 2600, 2700, 2750, 2800, 3900, 3000, 3500, 66,00, 7300, 3750, 3800, 3900, 4000, 5000, or 7500 mg.
103. The method of any one of the preceding arrangements, wherein the antibody is at a dose of at least 70, 140, or 1000 mg, or at a range that is defined by any two of the preceding values.
104. The method of any one of the preceding arrangements, wherein the antibody is at a dose of at least 70, 140, 150, 175, 200, 250, 300, 350, 420, 500, 1000, 1500, 2000, 2100, 2500, 3000, 3500, 4000, or 5000 mg, or at a range that is defined by any two of the preceding values.
105. The method of any one of the preceding arrangements, wherein the antibody is administered at a dose of at least 0.1, 0.5, 1, 2, 3, 4, 5, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 175, 200, 225, 250, 275, or 300 mg/kg, or by a range that is defined by any two of the preceding values.
106. The method of any one of the preceding arrangements, wherein the antibody is administered at a dose of at least 0.1, 2, 4, 6, 10, 14, 15, 20, 25, 30, 40, 50, 60, 70 mg/kg, or by a range that is defined by any two of the preceding values.
107. The method of any one of the preceding arrangements, wherein the antibody is administered at a dose of at least 2, 6, or 14 mg/kg, or by a range that is defined by any two of the preceding values.
108. The method of any one of the preceding claims, wherein the antibody is administered over the course of at least 10, 20, 30, 40, 50, 60, 70, 80, 90, 100, 120, 130, 140, 150, 160, 170, 180, 190, or 200 minutes, or by a range that is defined by any two of the preceding values.
109. The method of any one of the preceding arrangements, wherein the antibody is formulated at 20 mg/ml (in some embodiments, the 20 mg/ml can be diluted to 8 mg/ml in a total of 500 mL prior to I. V. administration).
110. The method of any one of the preceding arrangements, further comprising diluting the antibody prior to administration, wherein the antibody is diluted in sterile normal saline for injection.
111. The method of any one of the preceding arrangements, further comprising diluting a 20 mg/mL (in some embodiments, the 20 mg/ml can be diluted to 8 mg/ml in a total of 500 mL prior to I. V. administration) antibody formulation in an IV bag containing 0.9% sodium chloride and normal saline.
112. The method of any one of the preceding arrangements, wherein the antibody diluted in sterile normal saline is administered to the subject by IV infusion over one hour.
113. The method of any one of the preceding arrangements, wherein the antibody diluted in sterile saline is administered to the subjects at least once every 28 days.
114. The method of any one of the preceding arrangements, wherein the antibody is administered over the course of at least 60 minutes.
115. The method of any one of the preceding arrangements, wherein the wherein the antibody is administered at least every 2 days, 3 days, 4 days, 5 days, 6 days, 7 days, 10 days, 12 days, 15 days, 20 days, or 28 days, 29 days, 30 days, 31 days, or by a range that is defined by any two of the preceding values.
116. The method of any one of the preceding arrangements, wherein the wherein the antibody is administered at least every 28, 29, 30, and/or 31 days.
117. The method of any one of the preceding arrangements, wherein the antibody is administered at least 1, 2, 3, 4, 5, 6, 7, 8, 9, 10, 11, 12 times, or at an interval that is defined by any two of the preceding values.
118. The method of any one of the preceding arrangements, wherein the antibody is administered at least 1, 2, 3, 4, 5, 6, 7, 8, 9, or 10 times per 28 day cycle, or at an interval that is defined by any two of the preceding values.
119. The method of any one of the preceding arrangements, wherein the antibody is administered at least once per every 28 day cycle for up to 113 weeks.
120. The method of any one of the preceding arrangements, wherein the antibody is administered at least once per every 28 day cycle indefinitely.
121. The method of any one of the preceding arrangements, wherein the antibody is administered at least daily, weekly, bi-weekly, or every 10 days.
122. The method of any one of the preceding arrangements, wherein the antibody is administered monthly at a dose of 5000 mg.
123. The method of any one of the preceding arrangements, further comprising administration of anti-infectives, antibiotics, corticosteroids, opioid analgesics, anxiolytics, muscle relaxants, paracetamol, acetaminophen, or any combination thereof.
124. The method of any one of the preceding arrangements, further comprising administering one or more physical exams on the subject.
125. The method of any one of the preceding arrangements, further comprising evaluating the pharmacokinetics of the antibody in the subject's blood following administration.
126. The method of any one of the preceding arrangements, wherein the subject is at least 35, 40, 45, 50, 55, 60, 65, 60, 75, 80, 85, or 90 years old, or is at an age that is defined by any two of the preceding values.
127. The method of any one of the preceding arrangements, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein the antibody comprises at least 1 V_{L}-CDR having at least 90% identity to any one of the amino acid sequences of SEQ ID NOs: 170-296.
128. The method of any one of the preceding arrangements, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
   the antibody comprises at least 3 V_{L}-CDR having at least 90% identity to any one of the amino acid sequences of SEQ ID NOs: 170-296.
129. The method of any one of the preceding arrangements, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
   the antibody comprises at least 1 V_{H}-CDR having at least 90% identity to any one of the amino acid sequences of SEQ ID NOs: 27-169, 801, 802, 953, 954.
130. The method of any one of the preceding arrangements, wherein the antibody CDR3; and (2) a light chain variable region comprising a V_{L}-CDR1, a V_{L}-CDR2, and a V_{L}-CDR3, wherein
   the antibody comprises at least 3 V_{H}-CDRs having at least 90% identity to any one of the amino acid sequences of SEQ ID NOs: 27-169, 801, 802, 953, 954.
131. The method of any one of the preceding arrangements, wherein the antibody comprises at least 1 CDR having at least 90% identity to any one of the amino acid sequences of SEQ ID NOs: 27-296, 801, 802, 953, 954.
132. The method of any one of the preceding arrangements, wherein the antibody comprises at least 6 CDRs having at least 90% identity to any one of the amino acid sequences of SEQ ID NOs: 27-296, 801, 802, 953, 954.
133. The method of any one of the preceding arrangements, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the V_{H}-CDR1 comprises an amino acid sequence having at least 90% identity to any one of the amino acid sequences of SEQ ID NOs: 27-70;
the V_{H}-CDR2 comprises an amino acid sequence having at least 90% identity to any one of the amino acid sequences of SEQ ID NOs: 71-111, 801, 951, 952;
the V_{H}-CDR3 comprises an amino acid sequence having at least 90% identity to any one of the amino acid sequences of SEQ ID NO: 112-169, 802, 953, 954;
the V_{L}-CDR1 comprises an amino acid sequence having at least 90% identity to any one of the amino acid sequences of SEQ ID NOs: 170-220;
the V_{L}-CDR2 comprises an amino acid sequence having at least 90% identity to any one of the amino acid sequences of SEQ ID NOs: 221-247; and
the V_{L}-CDR3 comprises an amino acid sequence having at least 90% identity to any one of the amino acid sequences of SEQ ID NOs: 248-296.
134. The method or composition of any one of the preceding arrangements, wherein the antibody comprises a combination of the V_{H}-CDR1, V_{H}-CDR2, V_{H}-CDR3, V_{L}-CDR1, V_{L}-CDR2, and V_{L}-CDR3 as illustrated in FIG. 43.
135. The method or composition of any one of the preceding arrangements, wherein the heavy chain variable region comprises a sequence having at least 90% identity to the sequence selected from SEQ ID NOs: 297-373, 803, 806-820, 940, 955-968, 1067-1109, 1415-1439.
136. The method of any one of the preceding arrangements, wherein the light chain variable region comprises a sequence having at least 90% identity to the sequence selected from SEQ ID NOs: 374-447, 821-835, 941-943, 969-982, 1110-1152, 1440-1464.
137. The method of any one of the preceding arrangements, wherein the antibody comprises a heavy chain, wherein the heavy chain comprises a sequence having at least 90% identity to the sequence selected from SEQ ID NOs: 448-494, 804, 836-850, 983-996, 1153-1195, 1411, 1465-1489.
138. The method or composition of any one of the preceding arrangements, wherein the antibody comprises a light chain, wherein the light chain comprises a sequence having at least 90% identity to the sequence selected from SEQ ID NOs: 495-538, 805, 851-865, 997-1010, 1196-1238, 1412, 1490-1514.
139. The method of any one of the preceding arrangements, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
   the V_{H}-CDR1 comprises an amino acid sequence having at least 90% identity to SEQ ID NO: 31;
   the V_{H}-CDR2 comprises an amino acid sequence having at least 90% identity to SEQ ID NO: 72;
   the V_{H}-CDR3 comprises an amino acid sequence having at least 90% identity to SEQ ID NO: 113;
   the V_{H}-CDR1 comprises an amino acid sequence having at least 90% identity to SEQ ID NO: 170;
   the V_{H}-CDR1 comprises an amino acid sequence having at least 90% identity to SEQ ID NO: 221; and
   the V_{H}-CDR1 comprises an amino acid sequence having at least 90%, to SEQ ID NO: 248.
140. The method of any one of the preceding arrangements, wherein the antibody CDR3; and (2) a light chain variable region comprising a V_{L}-CDR1, a V_{L}-CDR2, and a V_{L}-CDR3, wherein
   the antibody comprises at least 3 V_{H}-CDRs having at least 90% identity to any one of the amino acid sequences of SEQ ID NOs: 31, 72, 113.
141. The method of any one of the preceding arrangements, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
   the antibody comprises at least 3 V_{H}-CDRs having at least 90% identity to any one of the amino acid sequences of SEQ ID NOs: 170, 221, 248.
142. The method or composition of any one of the preceding arrangements, wherein the heavy chain variable region comprises a sequence having at least 90% identity to SEQ ID NO: 298.
143. The method or composition of any one of the preceding arrangements, wherein the heavy chain variable region comprises a sequence having at least 90% identity to SEQ ID NO: 375.
144. The method of any one of the preceding arrangements, wherein the antibody comprises a heavy chain, wherein the heavy chain comprises a sequence having at least 90% identity to the SEQ ID NO: 449.
145. The method or composition of any one of the preceding arrangements, wherein the antibody comprises a light chain, wherein the light chain comprises a sequence having at least 90% identity to SEQ ID NO: 496.
146. The method of any one of the preceding arrangements, wherein the antibody is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.
147. The method of any one of the preceding arrangements, wherein the antibody is a TB006 antibody comprising SEQ ID NO: 449 and/or SEQ ID NO: 495; wherein the TB006 antibody is formulated as a clear to slightly opalescent, sterile solution at unit dose strength of 20 mg/mL (in some embodiments, the 20 mg/ml can be diluted to 8 mg/ml in a total of 500 mL prior to I. V. administration) in a 8 mL vial (160 mg total); wherein the solution is suitable for injection.
148. The method of any one of the preceding arrangements, wherein a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8 is administered to the subject once monthly via intravenous injection over the course of 1-hour for a total administered dose of 4000 mg.
149. The method of any one of the preceding arrangements, wherein a subject's the subject's global CDR-SB score is decreased by at least about 0.23 points from baseline within about 15 days of administration of a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8 via intravenous injection over the course of 1-hour for a total administered dose of 4000 mg.
150. The method of any one of the preceding arrangements, wherein a subject's the subject's global CDR-SB score is decreased by at least about 0.34 points from baseline within about 36 days of administration of a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8 via intravenous injection over the course of 1-hour for a total administered dose of 4000 mg.
151. The method of any one of the preceding arrangements, wherein a subject's the subject's global CDR-SB score is decreased by at least about 0.27 points from baseline within about 64 days of administration of a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8 via intravenous injection over the course of 1-hour for a total administered dose of 4000 mg.
152. The method of any one of the preceding arrangements, wherein a subject's the subject's global CDR-SB score is decreased by at least about 0.43 points from baseline within about 104 days of administration of a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8 via intravenous injection over the course of 1-hour for a total administered dose of 4000 mg.
153. The method of any one of the preceding arrangements, wherein a subject's MMSE score is significantly increased from baseline within about 36 days of administration of a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8 via intravenous injection over the course of 1-hour for a total administered dose of 4000 mg.
154. The method of any one of the preceding arrangements, wherein a subject's Memory CDR-SB subdomain score is decreased by at least 0.05-0.3 points within at least 64 days following administration of a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8 via intravenous injection over the course of 1-hour for a total administered dose of 4000 mg.
155. The method of any one of the preceding arrangements, wherein a subject's Orientation CDR-SB subdomain score is decreased by at least 0.05-0.3 points within at least 15 days following administration of a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8 via intravenous injection over the course of 1-hour for a total administered dose of 4000 mg.
156. The method of any one of the preceding arrangements, wherein a subject's Orientation CDR-SB subdomain score is decreased by at least 0.05-0.3 points within at least 36 days following administration of a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8 via intravenous injection over the course of 1-hour for a total administered dose of 4000 mg.
157. The method of any one of the preceding arrangements, wherein a subject's Community Affairs CDR-SB subdomain score is decreased by at least 0.05-0.3 points within at least 36 days following administration of a therapeutic formulation comprising 20 mg/ml (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8 via intravenous injection over the course of 1-hour for a total administered dose of 4000 mg.
158. The method of any one of the preceding arrangements, wherein a subject's Home and Hobbies CDR-SB subdomain score is decreased by at least 0.05-0.3 points within at least 104 days following administration of a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8 via intravenous injection over the course of 1-hour for a total administered dose of 4000 mg.
159. The method of any one of the preceding arrangements, wherein a subject's Judgement and Problem Solving CDR-SB subdomain score is decreased by at least 0.05-0.3 points within at least 104 days following administration of a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8 via intravenous injection over the course of 1-hour for a total administered dose of 4000 mg.
160. The method of any one of the preceding arrangements, wherein a subject's Personal Care CDR-SB subdomain score is decreased by at least 0.05-0.3 points within at least 104 days following administration of a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8 via intravenous injection over the course of 1-hour for a total administered dose of 4000 mg.
161. The method of any one of the preceding arrangements, wherein a subject's the subject's global CDR-SB score is decreased by at least about 0.5 points from baseline following about 3 months of once-monthly administration of a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8 via intravenous injection over the course of 1-hour for a total administered dose of 4000 mg.
162. The method of any one of the preceding arrangements, wherein the antibody comprises TB006, wherein TB006 comprises SEQ ID NO: 449 and SEQ ID NO: 496, wherein the antibody is administered on approximately a 1 week basis for at least a month, wherein the antibody is administered via IV, and wherein the antibody is administered at least 3, 4, or 5 times over the month.
163. The method of arrangement 147, wherein the amount of the antibody and/or formulation used is a) 4000 mg q28 day and/or b) 8 mg/mL in 500 mL, per administration.
164. The method of arrangement 148, wherein the IV infusion is over an hour.
165. The method of any one of the preceding arrangements, wherein
   the anti-Gal3 antibody is administered as a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8; and wherein
   the anti-Gal3 antibody is administered 5 times in a month via intravenous injection over the course of 1-hour for a total administered dose of 140-4000 mg.
166. The method of any one of the preceding arrangements, wherein
   the anti-Gal3 antibody is administered as a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8; and wherein
   the anti-Gal3 antibody is administered 5 times in a month via intravenous injection over the course of 1-hour for a total administered dose of 140 mg.
167. The method of any one of the preceding arrangements, wherein
   the anti-Gal3 antibody is administered as a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8; and wherein
   the anti-Gal3 antibody is administered 5 times in a month via intravenous injection over the course of 1-hour for a total administered dose of 420 mg.
168. The method of any one of the preceding arrangements, wherein
   the anti-Gal3 antibody is administered as a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8; and wherein
   the anti-Gal3 antibody is administered 5 times in a month via intravenous injection over the course of 1-hour for a total administered dose of 1000 mg.
169. The method of any one of the preceding arrangements, wherein
   the anti-Gal3 antibody is administered as a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methioninc, 100 mM NaCl, 0.02% PS80, and a pH of 5.8; and wherein
   the anti-Gal3 antibody is administered 5 times in a month via intravenous injection over the course of 1-hour for a total administered dose of 140 mg, followed by administration 5 times in a month via intravenous injection over the course of 1-hour for a total administered dose of 1000 mg.
170. The method of any one of the preceding arrangements, wherein
   the anti-Gal3 antibody is administered as a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8; and wherein
   the anti-Gal3 antibody is administered 5 times in a month via intravenous injection over the course of 1-hour for a total administered dose of 420 mg followed by administration 5 times in a month via intravenous injection over the course of 1-hour for a total administered dose of 1000 mg.
171. The method of any one of the preceding arrangements, wherein
   the anti-Gal3 antibody is administered as a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8; and wherein
   the anti-Gal3 antibody is administered 5 times in a month via intravenous injection over the course of 1-hour for a total administered dose of 1000 mg followed by administration 5 times in a month via intravenous injection over the course of 1-hour for a total administered dose of 1000 mg.
172. The method of any one of the preceding arrangements, wherein
   the anti-Gal3 antibody is administered as a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8; and wherein
   the anti-Gal3 antibody is administered 5 times in a month via intravenous injection over the course of 1-hour for a total administered dose of 140 mg, followed by administration once monthly, every 28 days, via intravenous injection over the course of 1-hour for a total administered dose of 4000 mg.
173. The method of any one of the preceding arrangements, wherein
   the anti-Gal3 antibody is administered as a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8; and wherein
   the anti-Gal3 antibody is administered 5 times in a month via intravenous injection over the course of 1-hour for a total administered dose of 420 mg, followed by administration once monthly, every 28 days, via intravenous injection over the course of 1-hour for a total administered dose of 4000 mg.
174. The method of any one of the preceding arrangements, wherein
   the anti-Gal3 antibody is administered as a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8; and wherein
   the anti-Gal3 antibody is administered 5 times in a month via intravenous injection over the course of 1-hour for a total administered dose of 1000 mg followed by administration once monthly, every 28 days, via intravenous injection over the course of 1-hour for a total administered dose of 4000 mg.
175. The method of any one of the preceding arrangements, wherein
   the anti-Gal3 antibody is administered as a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8; and wherein
   the anti-Gal3 antibody is administered 5 times in a month via intravenous injection over the course of 1-hour for a total administered dose of 140 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg.
176. The method of any one of the preceding arrangements, wherein
   the anti-Gal3 antibody is administered as a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8; and wherein
   the anti-Gal3 antibody is administered 5 times in a month via intravenous injection over the course of 1-hour for a total administered dose of 420 mg followed by administration 5 times in a month via intravenous injection over the course of 1-hour for a total administered dose of 1000 mg, followed by administration once monthly, every 28 days, via intravenous injection over the course of 1-hour for a total administered dose of 4000 mg.
177. The method of any one of the preceding arrangements, wherein
   the anti-Gal3 antibody is administered as a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8; and wherein
   the anti-Gal3 antibody is administered 5 times in a month via intravenous injection over the course of 1-hour for a total administered dose of 1000 mg followed by administration 5 times in a month via intravenous injection over the course of 1-hour for a total administered dose of 1000 mg followed by administration once monthly, every 28 days, via intravenous injection over the course of 1-hour for a total administered dose of 4000 mg.
178. A method of decreasing a subject's Clinical Dementia Rating Scale-Sum of Boxes (CDR-SB), the method comprising:
   identifying a subject as likely to benefit from a decreased CDR-SB score, and
   administering a therapeutically effective amount of an antibody, wherein
      the antibody binds to Gal3, and wherein
      the anti-Gal3 antibody is administered 5 times in a month via intravenous injection over the course of 1-hour for a total administered dose of 140-4000 mg, followed by administration once monthly, every 28 days, via intravenous injection over the course of 1-hour for a total administered dose of 140-4000 mg.
179. A method of increasing a subject's Mini Mental State Examination (MMSE) score, the method comprising:
   identifying a subject as likely to benefit from an increased MMSE score, and
   administering a therapeutically effective amount of an antibody, wherein
      the antibody binds to Gal3, and wherein
      the anti-Gal3 antibody is administered 5 times in a month via intravenous injection over the course of 1-hour for a total administered dose of 140-4000 mg, followed by administration once monthly, every 28 days, via intravenous injection over the course of 1-hour for a total administered dose of 140-4000 mg.
180. A method of decreasing a subject's Clinical Dementia Rating Scale-Sum of Boxes (CDR-SB), the method comprising:
   identifying a subject as likely to benefit from a decreased CDR-SB score, and
   administering a therapeutically effective amount of TB006 antibody, wherein
      the TB006 antibody is administered 5 times in a month via intravenous injection over the course of 1-hour for a total administered dose of 140-4000 mg, followed by administration once monthly, every 28 days, via intravenous injection over the course of 1-hour for a total administered dose of 140-4000 mg.
181. A method of increasing a subject's Mini Mental State Examination (MMSE) score, the method comprising:
   identifying a subject as likely to benefit from an increased MMSE score, and
   administering a therapeutically effective amount of TB006 antibody, wherein
      the TB006 antibody is administered 5 times in a month via intravenous injection over the course of 1-hour for a total administered dose of 140-4000 mg, followed by administration once monthly, every 28 days, via intravenous injection over the course of 1-hour for a total administered dose of 140-4000 mg.
182. A method of decreasing a subject's Clinical Dementia Rating Scale-Sum of Boxes (CDR-SB), the method comprising:
   identifying a subject as likely to benefit from a decreased CDR-SB score, and
   administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein
      the therapeutic formulation is administered 5 times in a month via intravenous injection over the course of 1-hour for a total administered dose of 140-4000 mg, followed by administration once monthly, every 28 days, via intravenous injection over the course of 1-hour for a total administered dose of 140-4000 mg.
183. A method of increasing a subject's Mini Mental State Examination (MMSE) score, the method comprising:
   identifying a subject as likely to benefit from an increased MMSE score, and
   administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein
      the therapeutic formulation is administered 5 times in a month via intravenous injection over the course of 1-hour for a total administered dose of 140-4000 mg, followed by administration once monthly, every 28 days, via intravenous injection over the course of 1-hour for a total administered dose of 140-4000 mg.
184. A method of decreasing a subject's Clinical Dementia Rating Scale-Sum of Boxes (CDR-SB), the method comprising:
   identifying a subject as likely to benefit from a decreased CDR-SB score, and
   administering a therapeutically effective amount of an antibody, wherein
      the antibody binds to Gal3, and wherein
      the anti-Gal3 antibody is administered 5 times in a month via intravenous injection over the course of 1-hour for a total administered dose of 140 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg.
185. A method of increasing a subject's Mini Mental State Examination (MMSE) score, the method comprising:
   identifying a subject as likely to benefit from an increased MMSE score, and
   administering a therapeutically effective amount of an antibody, wherein
      the antibody binds to Gal3, and wherein
      the anti-Gal3 antibody is administered 5 times in a month via intravenous injection over the course of 1-hour for a total administered dose of 140 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg.
186. A method of decreasing a subject's Clinical Dementia Rating Scale-Sum of Boxes (CDR-SB), the method comprising:
   identifying a subject as likely to benefit from a decreased CDR-SB score, and
   administering a therapeutically effective amount of TB006 antibody, wherein
      the TB006 antibody is administered 5 times in a month via intravenous injection over the course of 1-hour for a total administered dose of 140 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg.
187. A method of increasing a subject's Mini Mental State Examination (MMSE) score, the method comprising:
   identifying a subject as likely to benefit from an increased MMSE score, and
   administering a therapeutically effective amount of TB006 antibody, wherein
      the TB006 antibody is administered 5 times in a month via intravenous injection over the course of 1-hour for a total administered dose of 140 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg.
188. A method of decreasing a subject's Clinical Dementia Rating Scale-Sum of Boxes (CDR-SB), the method comprising:
   identifying a subject as likely to benefit from a decreased CDR-SB score, and
   administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein
      the therapeutic formulation is administered 5 times in a month via intravenous injection over the course of 1-hour for a total administered dose of 140 mg, followed by administration 5 times in a month for a total administered dose of 1000 mg, followed by administration once monthly, every 28 days, via intravenous injection over the course of 1-hour for a total administered dose of 4000 mg.
189. A method of increasing a subject's Mini Mental State Examination (MMSE) score, the method comprising:
   identifying a subject as likely to benefit from an increased MMSE score, and
   administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein
      the therapeutic formulation is administered 5 times in a month via intravenous injection over the course of 1-hour for a total administered dose of 140 mg, followed by administration 5 times in a month for a total administered dose of 1000 mg, followed by administration once monthly, every 28 days, via intravenous injection over the course of 1-hour for a total administered dose of 4000 mg.
190. A method of decreasing a subject's Clinical Dementia Rating Scale-Sum of Boxes (CDR-SB), the method comprising:
   identifying a subject as likely to benefit from a decreased CDR-SB score, and
   administering a therapeutically effective amount of an antibody, wherein
      the antibody binds to Gal3, and wherein
      the anti-Gal3 antibody is administered 5 times in a month via intravenous injection over the course of 1-hour for a total administered dose of 420 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg.
191. A method of increasing a subject's Mini Mental State Examination (MMSE) score, the method comprising:
   identifying a subject as likely to benefit from an increased MMSE score, and
   administering a therapeutically effective amount of an antibody, wherein
      the antibody binds to Gal3, and wherein
      the anti-Gal3 antibody is administered 5 times in a month via intravenous injection over the course of 1-hour for a total administered dose of 420 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg.
192. A method of decreasing a subject's Clinical Dementia Rating Scale-Sum of Boxes (CDR-SB), the method comprising:
   identifying a subject as likely to benefit from a decreased CDR-SB score, and
   administering a therapeutically effective amount of TB006 antibody, wherein
      the TB006 antibody is administered 5 times in a month via intravenous injection over the course of 1-hour for a total administered dose of 420 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg.
193. A method of increasing a subject's Mini Mental State Examination (MMSE) score, the method comprising:
   identifying a subject as likely to benefit from an increased MMSE score, and
   administering a therapeutically effective amount of TB006 antibody, wherein
      the TB006 antibody is administered 5 times in a month via intravenous injection over the course of 1-hour for a total administered dose of 420 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg.
194. A method of decreasing a subject's Clinical Dementia Rating Scale-Sum of Boxes (CDR-SB), the method comprising:
   identifying a subject as likely to benefit from a decreased CDR-SB score, and
   administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein
      the therapeutic formulation is administered 5 times in a month via intravenous injection over the course of 1-hour for a total administered dose of 420 mg, followed by administration 5 times in a month for a total administered dose of 1000 mg, followed by administration once monthly, every 28 days, via intravenous injection over the course of 1-hour for a total administered dose of 4000 mg.
195. A method of increasing a subject's Mini Mental State Examination (MMSE) score, the method comprising:
   identifying a subject as likely to benefit from an increased MMSE score, and
   administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein
      the therapeutic formulation is administered 5 times in a month via intravenous injection over the course of 1-hour for a total administered dose of 420 mg, followed by administration 5 times in a month for a total administered dose of 1000 mg, followed by administration once monthly, every 28 days, via intravenous injection over the course of 1-hour for a total administered dose of 4000 mg.
196. A method of decreasing a subject's Clinical Dementia Rating Scale-Sum of Boxes (CDR-SB), the method comprising:
   identifying a subject as likely to benefit from a decreased CDR-SB score, and
   administering a therapeutically effective amount of an antibody, wherein
      the antibody binds to Gal3, and wherein
      the anti-Gal3 antibody is administered 5 times in a month via intravenous injection over the course of 1-hour for a total administered dose of 1000 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg.
197. A method of increasing a subject's Mini Mental State Examination (MMSE) score, the method comprising:
   identifying a subject as likely to benefit from an increased MMSE score, and
   administering a therapeutically effective amount of an antibody, wherein
      the antibody binds to Gal3, and wherein
      the anti-Gal3 antibody is administered 5 times in a month via intravenous injection over the course of 1-hour for a total administered dose of 14000 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg.
198. A method of decreasing a subject's Clinical Dementia Rating Scale-Sum of Boxes (CDR-SB), the method comprising:
   identifying a subject as likely to benefit from a decreased CDR-SB score, and administering a therapeutically effective amount of TB006 antibody, wherein
      the TB006 antibody is administered 5 times in a month via intravenous injection over the course of 1-hour for a total administered dose of 1000 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg.
199. A method of increasing a subject's Mini Mental State Examination (MMSE) score, the method comprising:
   identifying a subject as likely to benefit from an increased MMSE score, and
   administering a therapeutically effective amount of TB006 antibody, wherein
      the TB006 antibody is administered 5 times in a month via intravenous injection over the course of 1-hour for a total administered dose of 1000 mg, followed by administration 5 times in a month via intravenous injection over the course of 1-hour for a total administered dose of 1000 mg, followed by administration once monthly, every 28 days, via intravenous injection over the course of 1-hour for a total administered dose of 4000 mg.
200. A method of decreasing a subject's Clinical Dementia Rating Scale-Sum of Boxes (CDR-SB), the method comprising:
   identifying a subject as likely to benefit from a decreased CDR-SB score, and
   administering a therapeutic formulation comprising 20 mg/ml (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein
      the therapeutic formulation is administered 5 times in a month via intravenous injection over the course of 1-hour for a total administered dose of 1000 mg, followed by administration 5 times in a month for a total administered dose of 1000 mg, followed by administration once monthly, every 28 days, via intravenous injection over the course of 1-hour for a total administered dose of 4000 mg.
201. A method of increasing a subject's Mini Mental State Examination (MMSE) score, the method comprising:
   identifying a subject as likely to benefit from an increased MMSE score, and
   administering a therapeutic formulation comprising 20 mg/mL (in some embodiments, the 20 mg/ml can be diluted to 8 mg/ml in a total of 500 mL prior to I. V. administration) TB006 antibody, 20 mM L-Histidine, 5 mM Methionine, 100 mM NaCl, 0.02% PS80, and a pH of 5.8, wherein
      the therapeutic formulation is administered 5 times in a month via intravenous injection over the course of 1-hour for a total administered dose of 1000 mg, followed by administration 5 times in a month for a total administered dose of 1000 mg, followed by administration once monthly, every 28 days, via intravenous injection over the course of 1-hour for a total administered dose of 4000 mg.
202. The method of any one of the preceding arrangements, wherein the subject's plasma Gal3 levels increase following administration of the anti-Gal3 antibody or binding fragment thereof as compared to plasma Gal3 levels in the subject prior to administration.
203. The method of any one of the preceding arrangements, wherein the subject's plasma Gal3 levels increase by up to about 120 ng/ml following administration of the anti-Gal3 antibody or binding fragment thereof as compared to plasma Gal3 levels in the subject prior to administration.
204. A method of diagnosing a subject as having, or likely having one or more diseases, the method comprising:
   administering an anti-Gal3 antibody or binding fragment thereof to a subject; wherein
   the anti-Gal3 antibody or binding fragment thereof comprises a detectable payload; and
   detecting the payload; wherein
   detection of the payload indicates that the subject has or likely has one or more diseases.
205. A method of diagnosing a subject as having, or likely having a neurological disorder, the method comprising:
   administering an anti-Gal3 antibody or binding fragment thereof to a subject; wherein
   the anti-Gal3 antibody or binding fragment thereof comprises a detectable payload; and
   detecting the payload; wherein
   detection of the payload indicates that the subject has or likely has a neurological disorder.
206. A method of diagnosing a subject as having, or likely having a proteopathy, the method comprising:
   administering an anti-Gal3 antibody or binding fragment thereof to a subject; wherein
   the anti-Gal3 antibody or binding fragment thereof comprises a detectable payload; and
   detecting the payload; wherein
   detection of the payload indicates that the subject has or likely has a proteopathy.
207. A method of identifying a subject as having, or likely having Alzheimer's disease (AD), the method comprising,
   administering an anti-Gal3 antibody or binding fragment thereof to a subject; wherein
      the anti-Gal3 antibody or binding fragment thereof comprises a detectable payload; and
   detecting the payload; wherein
      detection of the payload in the subject's brain indicates that the subject has or likely has AD.
208. A method of identifying the severity of Alzheimer's disease (AD) in a subject as having AD, the method comprising,
   administering an anti-Gal3 antibody or binding fragment thereof to a subject; wherein
      the anti-Gal3 antibody or binding fragment thereof comprises a detectable payload; and
   detecting the payload; wherein
      the level of payload detection in the subject's brain indicates the severity of the subject's AD.
   amount of an antibody, wherein
      the antibody binds to Gal3.
209. An anti-Gal3 antibody or binding fragment thereof for use in diagnosing a subject with Alzheimer's disease (AD); wherein
   the anti-Gal3 antibody or binding fragment thereof binds to one or more peptides of SEQ ID NOs: 3-26; wherein
   the anti-Gal3 antibody or binding fragment thereof comprises a detectable payload; and wherein
   detection of the anti-Gal3 antibody in the subject's brain is indicates that the subject has or likely has AD.
210. The method of any one of the preceding arrangements, wherein the payload is detected through MRI and/or PET scan.
211. The anti-Gal3 antibody or binding fragment thereof of any one of the preceding arrangements, wherein the detectable payload comprises radioactive substances (e.g. radioisotopes, radionuclides, radiolabels or radiotracers).
212. The anti-Gal3 antibody or binding fragment thereof of any one of the preceding arrangements, wherein the detectable payload comprises 18F, 18F-FAC, 32P, 33P, 45Ti, 47Sc, 52Fe, 59Fe, 62Cu, 64Cu, 67Cu, 67Ga, 68Ga, 75Sc, 77As, 86Y, 90Y, 89Sr, 89Zr, 94Tc, 94Tc, 99mTc, 99Mo, 105Pd, 105Rh, 111Ag, 111 In, 123I, 124I, 125I, 131I, 142Pr, 143Pr, 149Pm, 153Sm, 154-158Gd, 161Tb, 166Dy, 166Ho, 169Er, 175Lu, 177Lu, 186Re, 188Re, 189Re, 194Ir, 198Au, 199Au, 211At, 211Pb, 212Bi, 212Pb, 213Bi, 223Ra, and/or 225Ac, or any combination thereof.
213. The anti-Gal3 antibody or binding fragment thereof of any one of the preceding arrangements, wherein the one or more diseases comprise fibrosis, liver fibrosis, kidney fibrosis, cardiac fibrosis, pulmonary fibrosis, non-alcoholic fatty liver disease, non-alcoholic steatohepatitis, sepsis, atopic dermatitis, psoriasis, cancer, brain cancer, breast cancer, colorectal cancer, kidney cancer, liver cancer, lung cancer, pancreatic cancer, bladder cancer, stomach cancer, a hematological malignancy, or any combination thereof.
214. The anti-Gal3 antibody or binding fragment thereof of any one of the preceding arrangements, wherein the neurological disorder comprises inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, brain cancer, or any combination thereof.
215. The anti-Gal3 antibody or binding fragment thereof of any one of the preceding arrangements, wherein the proteopathy comprises Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, tauopathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Friedreich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, odontogenic (Pindborg) tumor amyloid, or any combination thereof.

### EXAMPLES

### Example 1: An Open-Label Long Term Extension Study to Assess the Safety of Anti-Gal3 Antibodies in Patients with Alzheimer's Disease

The TB006 nonclinical pharmacology program establishes its potential as a therapeutic agent for Alzheimer's Disease (AD) by neutralizing Galectin-3 (Gal-3), which regulates the production and accumulation of amyloid beta (Aβ and tau proteins and is overexpressed in AD. The preclinical data have shown evidence of addressing underlying discase pathology and improving cognition.

The preclinical safety profile of TB006 further supports the clinical investigation of TB006. Accordingly an open-label long term extension of the double-blind (Example 19, TB006AD2102) study in patients with AD was performed in order to evaluate the long-term safety and tolerability of monthly dose of TB006 in patients with AD.

Patients who have completed lead-in Protocol TB006AD2102 (either Part 1 or Part 2) were eligible to participate. Patients with different comorbidities like, diabetes, and hypertension were eligible and included. For some patients, enrollment into this study began immediately upon completion of the lead-in study. For those patients, the EoS procedures conducted in Protocol TB006AD2102 were used as baseline values for this study.

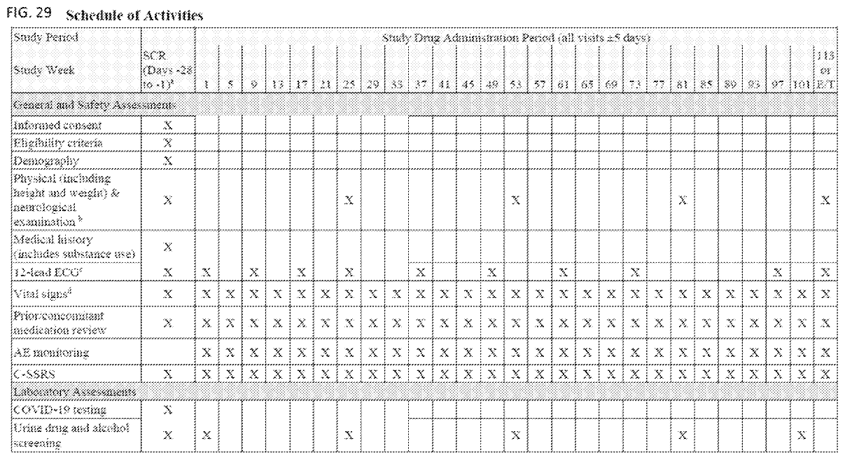

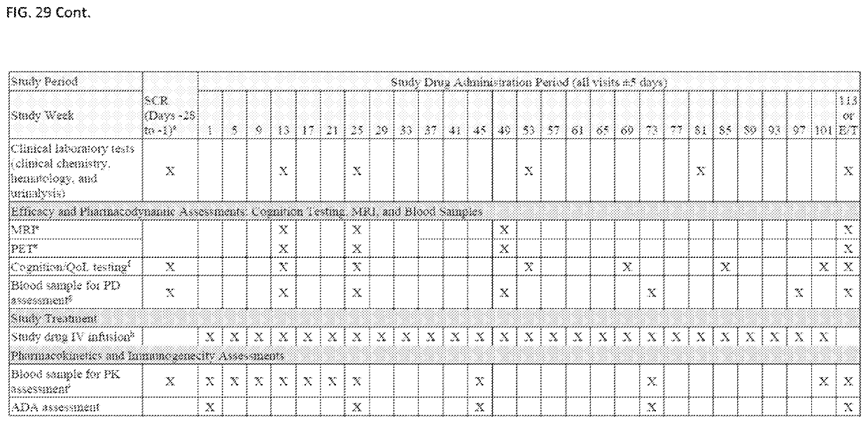

FIG. 29 is a table depicting a Schedule of Activities (SoA) according to visit number.

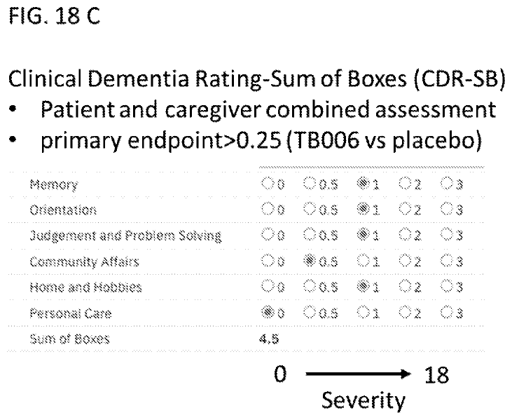

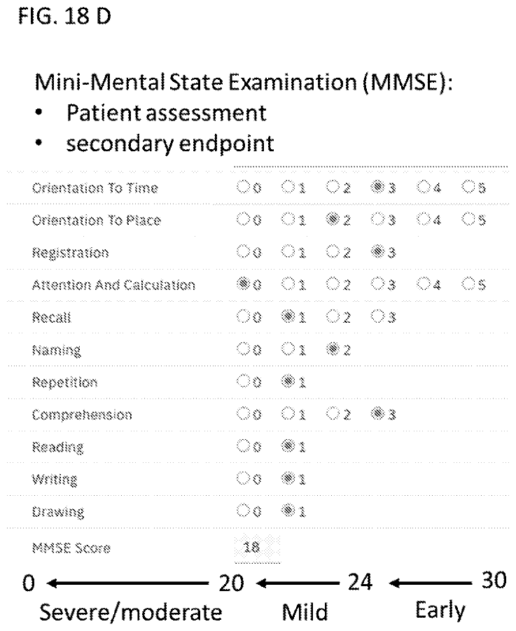

FIG. 18 is a flow chart depicting a method for testing the safety and efficacy of an anti-Gal3 antibody. FIG. 18A depicts some embodiments of a method for testing the safety and efficacy of an anti-Gal3 antibody.

The first dose may have been administered after completion of EoS procedures in Protocol TB006AD2102, either on the same day or within 28 days of the first dose for this study. The eligibility of patients who had completed the lead-in study before OLE was available and experienced a gap of more than 28 days between lead-in and OLE studies, was reconfirmed. These patients also underwent Screening procedures as outlined in the SoA (FIG. 29).

FIG. 27 is a table depicting study interventions administered to subjects.

FIG. 18B is a flow chart depicting a dosing and testing schedule.

In this study, the patients were administered TB006 according to FIG. 27. All patients received TB006 at a dose of 4000 mg via a 1-hour continuous IV infusion q28 day (±5 days). Patients were observed in the clinic for at least 2 hours following the end of the infusion for observation and safety assessments.

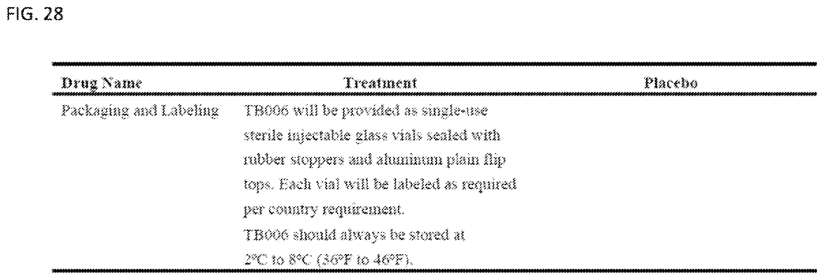

FIG. 28 is a table depicting packaging and labeling of an anti-Gal3 antibody.

TB006 was provided according to FIG. 28, as a sterilized liquid drug product was supplied in 8 mL glass vials, scaled with a rubber stopper and an aluminum flip top, containing 160 mg (20 mg/mL) (in some embodiments, the 20 mg/ml can be diluted to 8 mg/ml in a total of 500 mL prior to I. V. administration) TB006 drug product per vial. The TB006 was stored a 2C to 8C (36F to 46F).

The TB006 drug product was administered via IV infusion over 60 minutes, after adding the proper volume (200 mL) to an IV bag containing 300 mL of 0.9% Sodium Chloride Injection, USP (normal saline) for a total of 500 mL. The investigator or designee confirmed appropriate temperature conditions were maintained during transit for all study intervention received.

The does chosen were justified based on a SAD study (Protocol number TB006HV1101) that established the safety, tolerability, and PK of TB006 doses ranging from 70 to 5,000 mg in healthy volunteers. In the ongoing Phase 2 study in patients with AD, escalating doses up to 1,000 mg QW×5 doses (maximum total dose of 5,000 mg) were also safe and well tolerated). In both studies, exposures Cmax and AUC) at the highest dose afforded an 8- and 6-fold safety margin, respectively, from the exposures observed in the definitive GLP 1-month toxicology study.

The preclinical safety of TB006 chronic dosing was explored in a 6-month GLP toxicology study in cynomolgus monkeys. The doses used in this study were 3, 30, and 150 mg/kg, administered every two weeks for 6 months. This study included a 12-week recovery period and full histology.

Dosing throughout the study was well tolerated. There were no adverse findings of any type, either during the in-life phase or upon histology. Therefore, the NOAEL in cynomolgus monkeys dosed once biweekly for 6 months was 150 mg/kg. The mean steady state exposures associated with this dose across both males and females were Cmax of approximately 13,500 μg/mL and the (AUCO-τ) of approximately 1,990,000 h*ug/mL. Tau in the study was 2 weeks; when converted to 4 weeks as will be done in this study, the AUCO-τ is approximately 3,980,600 h*ug/mL.

The preclinical efficacy program established the potential of TB006 in several models of acute neurological and chronic neurodegenerative disorders. Although plasma therapeutic concentration ranges have not been established, the data indicate a positive dose-response relationship across these models. Therefore, the highest projected safe dose tested in the Phase 1 program will likely afford the greatest probability of success. For these reasons, a dose regimen of up to 4,000 mg q28 day is selected as the dose level for this study.

The mean steady-state Cmax and AUCO-τ from a dose of 4,000 mg q28 day (q28 day) are projected from non-parametric super positioning concentrations. The regimens were simulated up to 10,080 hours and took 9,408-10,080 hours for a multi-compartment model. These values are 2,640 μg/mL and 1,138,500 h*ug/mL, respectively. These values afford a 5-fold margin from the NOAEL on Cmax and a 3.5-fold margin on AUCO-τ at the planned dose of 4000 mg q28 day, adequate margins given the absence of any safety signals in the clinical studies to date.

When patients were dosed at the site, they received the study intervention directly from the investigator or designee, under medical supervision. The date and time of each dose administered in the clinic was recorded in the source documents. The dose of study intervention and study patient identification were confirmed at the time of dosing by a member of the study site staff other than the person administering the study intervention.

A record of the quantity of TB006 dispensed to and administered by each participant was maintained and reconciled with study intervention and compliance records. Intervention start and stop dates, including dates for intervention delays, were also recorded.

Patients returned to the clinic q28 day for dose administration and safety assessments. All visits will had a window of 11 days (±5 days from scheduled visit). The window was based on an original visit schedule planned from the start of the study. If a patient deviated within the window, the original schedule was retained. If a patient missed a visit or was outside the window, the visit was typically skipped and the monitor was notified for further instructions.

The efficacy assessment scale(s) selected for this study are also the list of scales for the efficacy endpoints. Planned timepoints for all efficacy assessments are provided in the SoA (FIG. 29).

Patients returned to the clinic over the course of approximately 3 months following the last dose according to FIG. 29 for the follow-up visit, which included safety assessments, cognition testing, QOL, imaging (PET and MRI), PD, and PK sampling.

If a patient reported any adverse events (AEs), they were required to return to the clinical unit at the discretion of the investigator. All adverse events were followed to adequate resolution.

The primary safety and tolerability endpoints for this study included: incidence of AEs and SAEs throughout the study; summary of ECGs throughout the study; summary of clinical laboratory assessments throughout the study; summary of vital signs assessments throughout the study; summary of physical and neurological examinations throughout the study.

All AEs were coded using the most recent version of the MedDRA. The incidence of AEs was summarized by SOC and PT. Similar summaries were produced for AEs by severity, SAEs, treatment related AEs, and AEs leading to discontinuation. AEs were collected from the first dose of study drug; thus, all AEs were considered treatment emergent AEs.

The ECG results were presented by visit, including the QT, QTc, QRS, RR, PR intervals, as well as change from baseline values, and clinically significant changes in heart rate and rhythm.

Reported values and change from baseline values of hematology, clinical chemistry, and urinalysis (and the determinations relevant to the normal ranges and appropriate clinically significant or CTCAE toxicity gradings), were summarized by laboratory test for each assessment day, using appropriate descriptive/summary statistics.

Vital signs (systolic and diastolic blood pressure, orthostatic blood pressure, pulse rate, respiratory rate, and oral temperature) were summarized by assessment day showing absolute values, change from baseline values, and clinically significant changes (including orthostasis) using appropriate descriptive statistics.

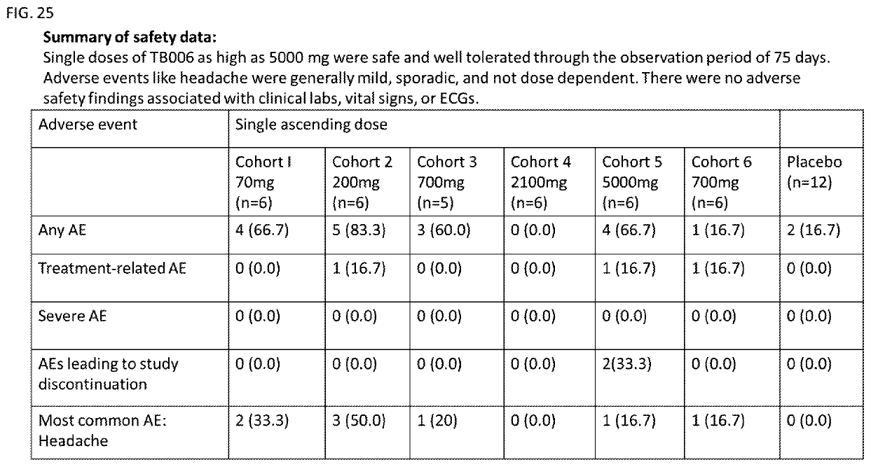

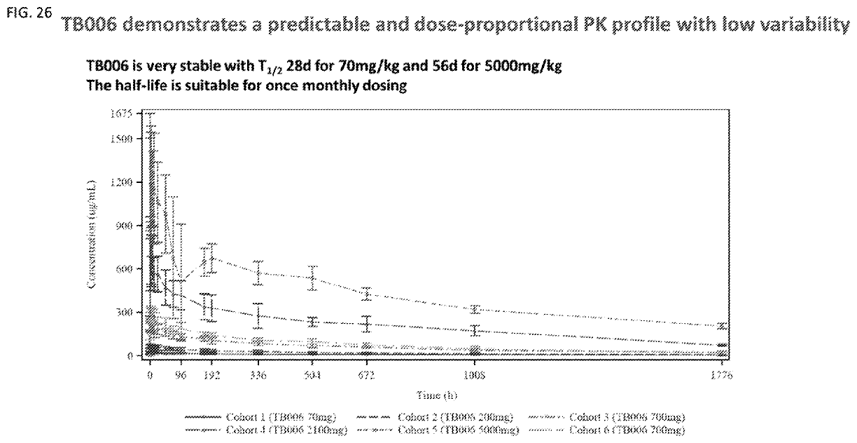

FIG. 25 is a table depicting the adverse events and their frequencies in a blinded cohort of the subject's treated with anti-Gal3 antibody.

As is clear from FIG. 25, single doses of TB006 as high as 5000 mg were safe and well tolerated through the observation period of 75 days. Adverse events like headache were generally mild, sporadic, and not dose dependent. There were no adverse safety findings associated with clinical labs, vital signs, or ECGs.

### Example 2: Anti-Gal3 Antibodies Promote Decreased Clinical Dementia Rating Scale-Sum of Boxes (CDR-SB) Scores

The longitudinal effect of TB006 on cognition was assessed in subjects undergoing an open-label long term extension of the double-blind (TB006AD2102) study in patients with AD according to the protocol of Example 1, above.

Approximately every 3 months, patients performed a battery of cognition and quality of life tests, and biomarker testing, including the Clinical Dementia Rating Scale-Sum of Boxes (CDR-SB), the Mini Mental State Examination (MMSE, Cognitive Drug Research system (CDR) battery, and the Neuropsychiatry Inventory (NPI), and the EuroQuality of life (EuroQol) 5-Dimension 5-Level (EQ-5D-5L).

FIG. 18C depicts a Clinical Dementia Rating-Sum of Boxes (CDR-SB) score keeping template.

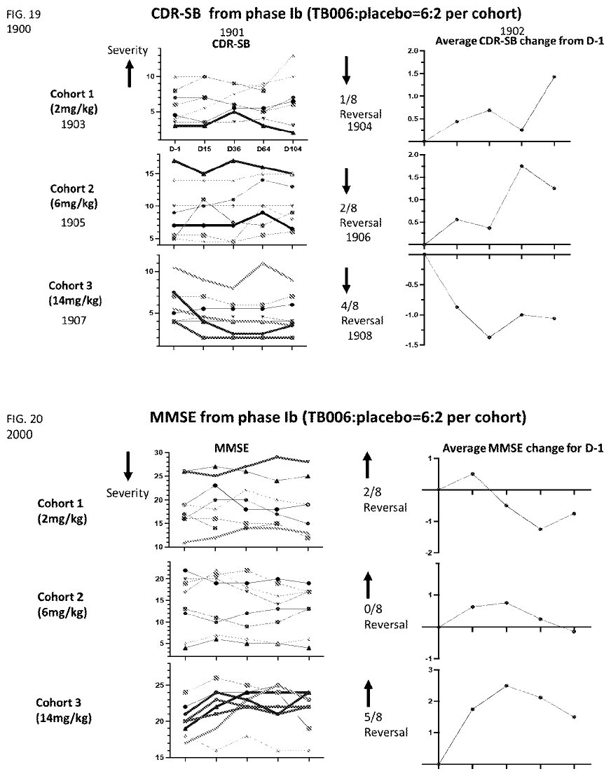

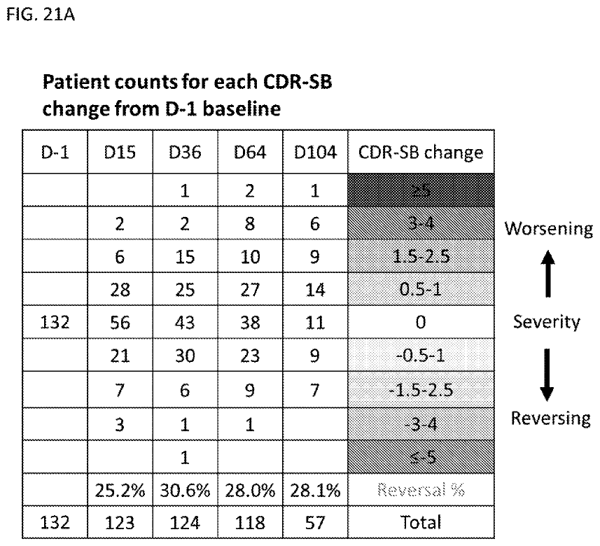

FIG. 19 depicts graphs of a subject's CDR-SB score following administration of an anti-Gal3 antibody at different doses.

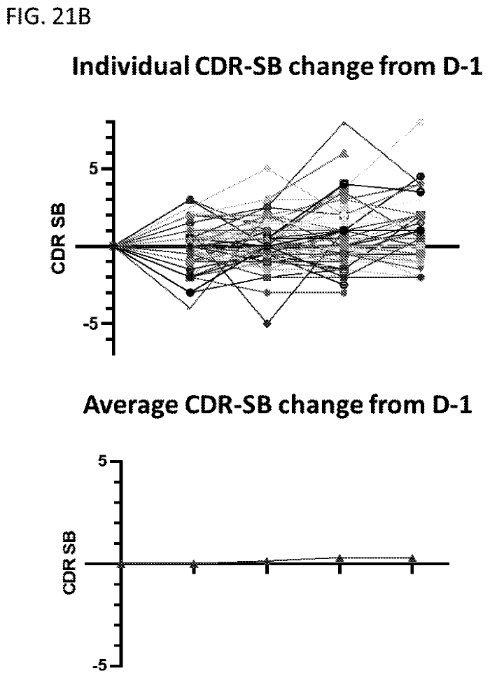

FIG. 21 depicts data from a cohort of subjects treated with an anti-Gal3 antibody. FIG. 21A is a heat map depicting some embodiments of the magnitude of change in a subject's CDR-SB score following administration of an anti-Gal3 antibody. FIG. 21B are embodiments of graphs depicting a subject's CDR-SB score following administration of an anti-Gal3 antibody.

As is clear from FIG. 19 and FIG. 21, administration of anti-Gal3 antibodies to a subject results in decreased, i.e., improved, CDR-SB scores.

### Example 3: Anti-Gal3 Antibodies Promote Increased Mini-Mental State Examination Scale (MMSE) Scores

The longitudinal effect of anti-Gal3 antibodies on cognition was assessed in subjects undergoing an open-label long term extension of the double-blind (TB006AD2102) study in patients with AD according to the protocol of Example 1, above.

Over approximately 3 months, patients performed a battery of cognition and quality of life tests, and biomarker testing, including the Clinical Dementia Rating Scale-Sum of Boxes (CDR-SB), the Mini Mental State Examination (MMSE, Cognitive Drug Research system (CDR) battery, and the Neuropsychiatry Inventory (NPI), and the EuroQuality of life (EuroQol) 5-Dimension 5-Level (EQ-5D-5L).

FIG. 18D depicts a Mini-Mental State Examination (MMSE) score keeping template.

FIG. 20 depicts graphs of a subject's MMSE score following administration of an anti-Gal3 antibody at different doses.

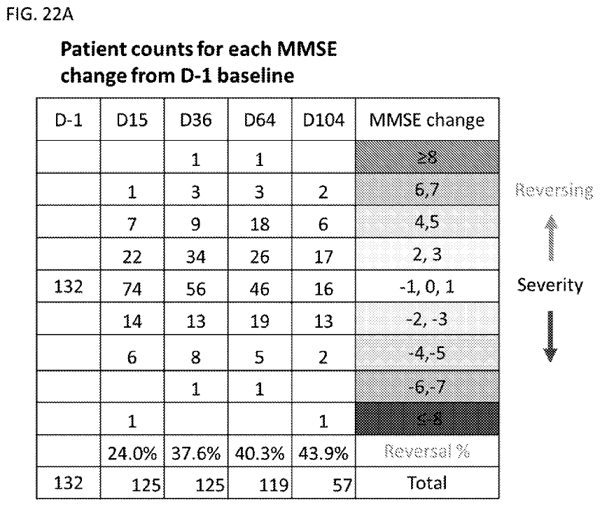

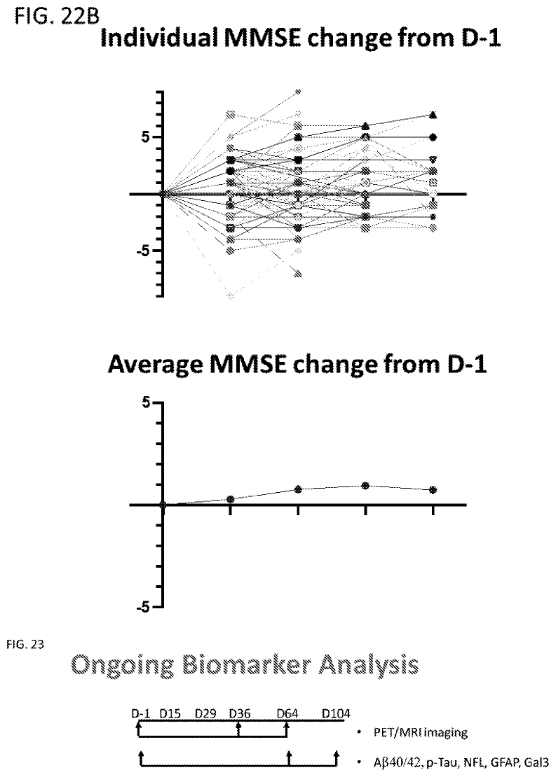

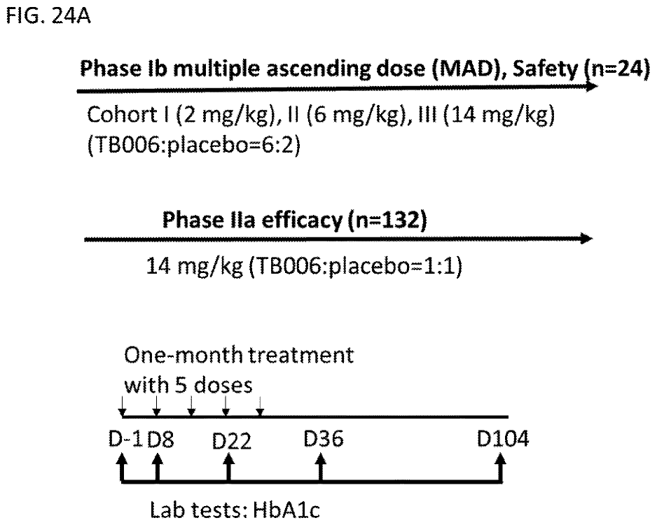

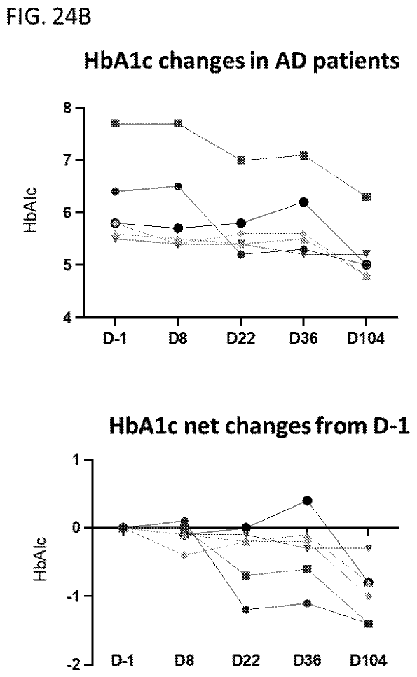

FIG. 22 depicts data from a cohort of subjects treated with an anti-Gal3 antibody. FIG. 22A is a heat map depicting some embodiments of the magnitude of change in a subject's MMSE score following administration of an anti-Gal3 antibody. FIG. 22B are graphs depicting embodiments of subject's MMSE score following administration of an anti-Gal3 antibody.

As is clear from FIG. 20 and FIG. 22, administration of anti-Gal3 antibodies to a subject results in increased, i.e., improved, MMSE scores.

### Example 4: Assessment of Plasma Biomarkers in Subjects Treated with an Anti-Gal3 Antibody

The effect of an anti-Gal3 antibody on plasma biomarkers Aβ40, tau (phosphorylated), neurofilament light chain (NFL), neurofilament heavy chain (NFH), and Galectin-3 (Gal-3), will be evaluated in AD patients undergoing an open-label long term extension of the double-blind (TB006AD2102) study according to example 1 above.

Venous blood samples of approximately 6 mL are collected for measurement of plasma Aβ40, tau (phosphorylated), NFL, NFH, Gal-3, and other relevant biomarkers at the timepoints specified in the SoA (FIG. 29).

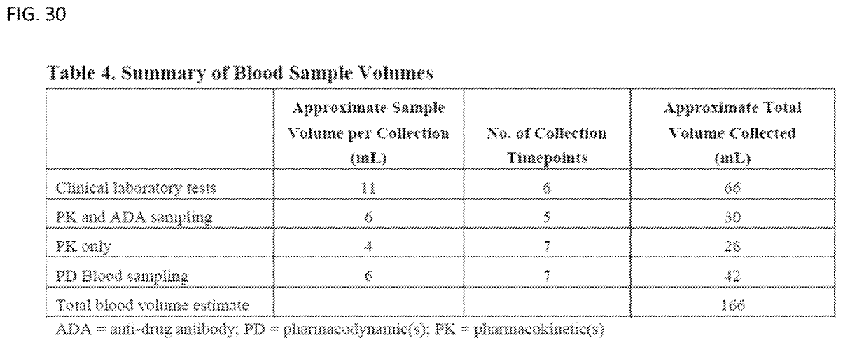

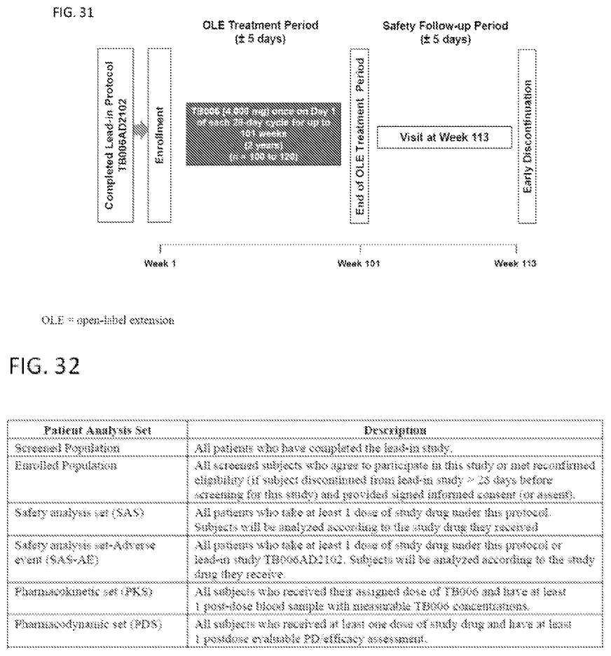

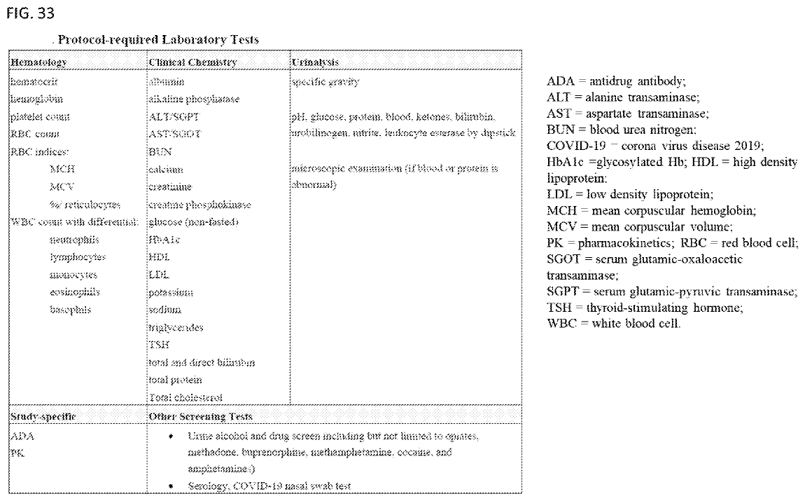

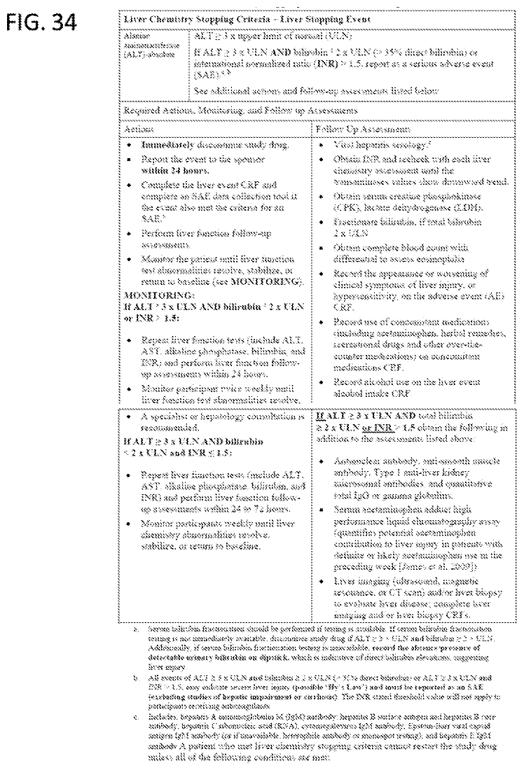

FIG. 30 is a table depicting blood volumes required for various assessments.

As can be seen in FIG. 30, The maximum amount of blood collected from each patient over the duration of the study, including any extra assessments that may be required, is not exceed 166 mL. Repeat or unscheduled samples may be taken for safety reasons or for technical issues with the samples.

It is expected that the administration of TB006 to AD patients undergoing an open-label long term extension of the double-blind (TB006AD2102) study according to example 1 above, results in decreased levels of plasma biomarkers Aβ40, tau (phosphorylated), neurofilament light chain (NFL), neurofilament heavy chain (NFH), and Galectin-3 (Gal-3).

### Example 5: Assessment of Brain Atrophy and Amyloid Plaque Formation in Subjects Treated with an Anti-Gal3 Antibody

Brain atrophy is measured, by volumetric measurements, as well amyloid plaque formation in AD patients undergoing an open-label long term extension of the double-blind (TB006AD2102) study according to example 1 above. Brain atrophy measured by volumetric measurements is assessed through magnetic resonance imaging (MRI). Amyloid plaque formation is assessed using positron emission tomography (PET).

Patients undergo imaging (PET and MRI) testing at months 3, 6, 12, and 24. These visits and all study assessments are presented in the SoA (FIG. 29).

The MRI is administered at the time points indicated in the SoA (FIG. 29). During the MRI, patients are instructed to lie still in an enclosed environment for the duration, which is usually approximately 20 minutes. Patients are informed that they may hear a buzzing sound throughout the procedure and may hear an occasional thumping sound. Otherwise, the procedure is noninvasive and painless. After the procedure, patients may need a few minutes to reorient themselves in a sitting position.

The PET scan is performed at the same time points as the MRI, as indicated in the SoA (FIG. 29). Patients undergoing PET scans may be asked to fast for 6 hours prior to the procedure, depending on the requirements of the facility. They may also be asked to reduce carbohydrate intake for 24 hours prior to the procedure. A central reader may be used in this study to interpret MRI and PET changes in amyloid deposits, volume, and other important clinical endpoints.

It is expected that the administration of TB006 to AD patients undergoing an open-label long term extension of the double-blind (TB006AD2102) study according to example 1 above, results in decreased brain atrophy and amyloid plaque formation.

### Example 6: Anti-Gal 3 Antibodies Demonstrates a Predictable and Dose-Proportional PK Profile with Low Variability

The pharmacokinetics of an anti-Gal3 antibody were evaluated in AD patients undergoing an open-label long term extension of the double-blind (TB006AD2102) study according to example 1 above.

FIG. 30 is a table depicting blood volumes required for various assessments.

As can be seen in FIG. 30, The maximum amount of blood collected from each patient over the duration of the study, including any extra assessments that may be required, did not exceed 166 mL. Repeat or unscheduled samples may have be taken for safety reasons or for technical issues with the samples.

Whole blood samples of approximately 4 mL was collected for measurement of plasma concentrations of TB006 as specified in the SoA (FIG. 29). Samples were used to evaluate the PK of TB006. Each plasma was divided into 2 aliquots (1 each for PK and a backup). Samples collected for analyses of TB006 (plasma) concentration were also to used to evaluate safety or efficacy aspects related to concerns arising during or after the study.

FIG. 26 is a graph showing a pharmacokinetic analysis of an anti-Gal3 antibody.

As is clear from FIG. 26, TB006 demonstrates a predictable and dose-proportional PK profile with low variability. TB006 is very stable with T½ 28 d for 70 mg/kg and 56 d for 5000 mg/kg. The half-life is suitable for once monthly dosing.

### Example 7: Assessment of the Immunogenicity of Anti-Gal 3 Antibodies

The immunogenicity of an anti-Gal3 antibody is evaluated in AD patients undergoing an open-label long term extension of the double-blind (TB006AD2102) study according to example 1 above.

Antibodies to TB006 are also evaluated in plasma samples collected from all patients according to the SoA (FIG. 29). Additionally, plasma samples are collected at the final visit from patients who discontinued study intervention or are withdrawn from the study.

Anti-TB006 antibodies are determined on plasma samples collected for plasma PK determinations, and no additional blood samples need to be obtained. Plasma samples are screened for antibodies binding to TB006 and the titer of confirmed positive samples is reported. Other analyses are performed to verify the stability of antibodies to TB006 and/or further characterize the immunogenicity of TB006.

The detection and characterization of antibodies to TB006 is performed using a validated assay method by or under the supervision of the sponsor. All samples collected for detection of antibodies to study intervention are also be evaluated for TB006 plasma concentration to enable interpretation of the antibody data. Antibodies are further characterized and/or evaluated for their ability to neutralize the activity of the study drug(s).

FIG. 30 is a table depicting blood volumes required for various assessments.

As can be seen in FIG. 30, The maximum amount of blood collected from each patient over the duration of the study, including any extra assessments that may be required, does not exceed 166 mL. Repeat or unscheduled samples may be taken for safety reasons or for technical issues with the samples.

It is expected that the administration of TB006 to AD patients undergoing an open-label long term extension of the double-blind (TB006AD2102) study according to example 1 above, will result induce an immune response.

### Example 8: Assessment of Safety and Efficacy of Anti-Gal 3 Antibodies in Patients with Moderate to Severe Alzheimer's Disease

Background: Galectin-3 has been reported to be highly expressed in Alzheimer's disease (AD) brain tissues. Our studies elucidated its intrinsic ability of acting as glue to promote oligomerization of Abeta, pTAU and other amyloid proteins in vitro. It's antagonist monoclonal antibodies showed dramatic cognition improvement and plaque reduction in AD mice after only two-week treatment. Dosing of the clinical lead antibody TB006 in a single ascending dose (SAD) study in healthy volunteers up to 5000 mg (70 mg/kg) was safe and well tolerated. This 2-part study was conducted in moderate to severe AD patients to assess the safety, tolerability, PK and efficacy of weekly TB006 doses.

Methods: This was a seamless Phase 1b/2a double-blinded, placebo controlled, multicenter study. AD patients with a screening MMSE<24 and without confounding neurologic or psychiatric disease were eligible. In Part 1, 3 groups (140 mg, 420 mg, 1000 mg) of 8 patients in sequential escalating fashion received either weekly TB006 (6) or placebo (2) infusions for 5 doses. In Part 2, participants were randomized (1:1) to receive either TB006 (1000 mg) or placebo weekly for 5 doses. Part 2 used the clinical dementia rating-sum of boxes (CDR-SB) score as the primary endpoint. Other endpoints were the mini-mental state examination (MMSE), neuropsychiatric inventory (NPI), CDR battery and plasma and imaging (MRI/PET) biomarkers. Cognition testing was done at baseline and on Days 15, 36, 64, and 104. Safety assessments were conducted at each visit. The sample size provided 80% power to detect a mean difference between TB006 and placebo of 0.25 point at Day 104 on the CDR-SB.

Results: 157 patients, including 24 in Part 1, were randomized at 15 US sites. Nine subjects prematurely discontinued. TB006 was safe and well tolerated at all dose levels. There were 9 severe adverse events (SAEs), including 1 death. None were related to TB006 treatment. Most other AEs were mild, sporadic and self-limiting. Patients in Part 1, Group 3 (1000 mg), as well as all Part 1 placebo patients were included in the efficacy analysis. The primary endpoint was met. Patients receiving TB006 showed a dramatic reduction on the CDR-SB score compared with placebo (p<0.01). Secondary efficacy endpoints were equally robust. Mean efficacy endpoint scores in the placebo group remained consistent throughout the observation period.

Conclusion: TB006 demonstrated evidence of AD reversal in this short-term treatment study. TB006 was safe and well tolerated.

### Example 9: Anti-Gal3 Antibodies Promote Increased EuroQol 5-Dimension 5-Level (EQ-5D-5L) Scores

The longitudinal effect of TB006 on cognition was assessed in subjects undergoing an open-label long term extension of the double-blind (TB006AD2102) study in patients with AD according to the protocol of Example 1, above.

Approximately every 3 months, patients performed a battery of cognition and quality of life tests, and biomarker testing, including the Clinical Dementia Rating Scale-Sum of Boxes (CDR-SB), the Mini Mental State Examination (MMSE, Cognitive Drug Research system (CDR) battery, and the Neuropsychiatry Inventory (NPI), and the EuroQuality of life (EuroQol) 5-Dimension 5-Level (EQ-5D-5L).

The EQ-5D-5L is a standardized instrument for use as a measure of health outcome. Mobility, self-care, usual activities, pain/discomfort, and anxiety/depression are each assessed on 5-point categorical scales ranging from “no problem” to “severe problem”. Two different administrations of the EQ-5D-5L will be performed at baseline and timepoints indicated in the SoA (FIG. 29).

First, the participant's caregiver will provide a proxy-rating of the patient's health status. Second, the caregiver will provide a self-report of his or her own health status. In both situations, the EQ-5D-5L questionnaire will be completed by the participant's caregiver.

It is expected that the administration of TB006 to AD patients undergoing an open-label long term extension of the double-blind (TB006AD2102) study according to example 1 above, will result in increased EuroQol 5-Dimension 5-Level (EQ-5D-5L) scores.

### Example 10: Anti-Gal3 Antibodies Promote Decreased Neuropsychiatric Inventory (NPI) Scores

The longitudinal effect of TB006 on cognition was assessed in subjects undergoing an open-label long term extension of the double-blind (TB006AD2102) study in patients with AD according to the protocol of Example 1, above.

Approximately every 3 months, patients performed a battery of cognition and quality of life tests, and biomarker testing, including the Clinical Dementia Rating Scale-Sum of Boxes (CDR-SB), the Mini Mental State Examination (MMSE, Cognitive Drug Research system (CDR) battery, and the Neuropsychiatry Inventory (NPI), and the EuroQuality of life (EuroQol) 5-Dimension 5-Level (EQ-5D-5L).

The NPI is a rater-administered, fully structured interview in which all questions are provided and read verbatim. The sole source of information is the interview with a caregiver who knows the patient well. This study uses the NPI version with 10 behavioral domains: delusions, hallucinations, agitation/aggression, depression/dysphoria, anxiety, elation/euphoria, apathy/indifference, disinhibition, irritability/lability, and aberrant motor behavior. The NPI total score is calculated by adding the scores of the domains (each domain scores range from 0 to 12). The NPI total score ranges from 0 to 120 with higher scores indicating greater behavioral impairment. The NPI-D scores in each of the domains are not included in the NPI total score. The NPI-D total score is calculated by adding the scores of caregiver distress in each of the domains (score ranges from 0 to 5 in each domain). The NPI-D total score ranges from 0 to 50 with higher scores indicating greater distress.

The NPI is administered at the time points indicated in the SoA (FIG. 29). The NPI is administered by an independent, trained, and certified member of the investigational team. At each site, the same individual, whenever possible, performs the NPI evaluation on a specific patient throughout the study.

It is expected that the administration of TB006 to AD patients undergoing an open-label long term extension of the double-blind (TB006AD2102) study according to example 1 above, will result in decreased NPI scores.

### Example 11: Anti-Gal3 Antibodies Promote Increased Cognitive Drug Research Battery Score Scores

The longitudinal effect of TB006 on cognition was assessed in subjects undergoing an open-label long term extension of the double-blind (TB006AD2102) study in patients with AD according to the protocol of Example 1, above.

Approximately every 3 months, patients performed a battery of cognition and quality of life tests, and biomarker testing, including the Clinical Dementia Rating Scale-Sum of Boxes (CDR-SB), the Mini Mental State Examination (MMSE, Cognitive Drug Research system (CDR) battery, and the Neuropsychiatry Inventory (NPI), and the EuroQuality of life (EuroQol) 5-Dimension 5-Level (EQ-5D-5L).

The CDR is an automated battery amenable to measurement of cognitive deficits in patients with AD. It consists of performance tasks measuring attention, working memory, episodic memory, and executive function. It contains 11 tests and is performed on a tablet-like device. The average duration of the battery is approximately 25 minutes.

The CDR is performed at the time points listed in the SoA (Table 1). Two training sessions will be conducted at least 30 minutes apart during the screening period to allow patients to become familiar with the procedure, reduce test anxiety, reduce learning effects, and produce a stable baseline assessment.

It is expected that the administration of TB006 to AD patients undergoing an open-label long term extension of the double-blind (TB006AD2102) study according to example 1 above, will result in increased Cognitive Drug Research battery score scores.

### Example 12: Anti-Gal3 Antibodies Promote Increased Whole Brain Volume

The effect of TB006 on whole brain volume was assessed in subjects undergoing an open-label long term extension of the double-blind (TB006AD2102) study in patients with AD according to the protocol of Example 1, above.

Subject 117-002's whole brain volume was assessed by MRI. The MRI was be administered at the time points indicated in the SoA (FIG. 29). The procedure was be performed at a center associated with site 117. Site staff coordinated the scheduling of the MRI and may have been asked to accompany the patient and caregiver to the facility. The MRI was scheduled after other screening procedures had been completed to ensure a high likelihood of patient eligibility.

During the MRI, subject 117-002 was instructed to lie still in an enclosed environment for the duration, which was usually approximately 20 minutes. The subject was informed that they may hear a buzzing sound throughout the procedure and may hear an occasional thumping sound.

Otherwise, the procedure was noninvasive and painless. After the procedure, the subject may have needed a few minutes to reorient themselves in a sitting position.

Table 6 depicts the whole brain volume of Subject 117-002 at 3 different time points following anti-Gal3 antibody administration.

TABLE 6

| Obs | SCAN_NUMBER | VISIT_ID | SCAN_DATE | MODALITY | BRAIN_REGION | VOLUME |
|---|---|---|---|---|---|---|
| 1338 | 1 | Screening | 22 Feb. 2022 | MRI | WHOLE_BRAIN | 9776.74 |
| 1348 | 2 | Day 36 | 27 Apr. 2022 | MRI | WHOLE_BRAIN | 9893.73 |
| 1358 | 3 | Day 64 | 24 May 2022 | MRI | WHOLE_BRAIN | 9832.33 |

As is clear from Table 6, anti-Gal3 antibodies promote increased whole brain volume within at least 36 and 64 days of anti-Gal3 antibody administration.

### Example 13: a Phase 1b/2a, Multi-Dose, Multi-Center Study to Assess the Safety, Tolerability, Pharmacokinetics, Pharmacodynamics, and Efficacy of TB006 in Patients with Alzheimer's Disease

A Seamless Phase 1b/2a Double-blind, Randomized, Multiple dose, Multi-center, Sequential Dose-escalation Study was conducted to Assess the Safety, Tolerability, Pharmacokinetics, Pharmacodynamics and Efficacy of TB006 in Patients with Mild to Severe Alzheimer's Disease.

Rationale: TB006 is an IgG4 type humanized antibody that is highly selective and has high affinity (8.4 nM) for human Gal-3. In vitro studies have shown that TB006 has virtually no affinity to any other tested protein. The TB006 nonclinical pharmacology program establishes its potential as a therapeutic agent for AD by neutralizing Gal-3, which regulates the production and accumulation of Aβ and tau proteins and is overexpressed in AD. In vivo preclinical studies in 10-month-old transgenic mice have demonstrated a pro-cognitive response in a variety of models and nearly total clearance of amyloid pathology on histology, with only 2 weeks of treatment at a dose of 10 mg/kg. GLP toxicology showed no adverse effects, cither clinically or histologically, in 1-month or 6-month studies at doses up to 200 and 150 mg/kg, respectively. Single doses of up to 5000 mg TB006 have been administered safely to healthy volunteers with few, if any, treatment-related adverse effects. The pharmacokinetic (PK) profile in plasma and cerebral spinal fluid (CSF) is characterized by low variability and predictable linearity over the dose range. The preclinical data have shown evidence of addressing underlying disease pathology and improving cognition.

Risk Assessment: A standard battery of toxicology studies was conducted with TB006. The pivotal safety assessment was a 1 month GLP toxicology study in cynomolgus monkeys. Doses ranged from 10 to 200 mg/kg, and evaluation included clinical and laboratory observations, a 5 week recovery period, and full histopathology. No treatment related adverse findings were observed. Mild lymphoid hyperplasia occurred in males at the 10, 100, and 200 mg/kg dose levels, but based on mild severity, lack of dose dependency and historical test facility data from similar studies in cynomolgus monkeys, this finding was considered a species specific mild, reactive immune response and not an adverse toxicological finding. It is likely an immunogenicity reaction. No other findings were observed in this or any of the other toxicology studies.

TB006 is a first in class monoclonal antibody, therefore there are no known class adverse effects or risks to report from clinical studies. In general, risks associated with other monoclonal antibodies include allergic reactions such as hives or itching, flu like symptoms such as chills, fatigue, fever, and muscle aches and pains, nausea and vomiting, skin rashes and decreases in blood pressure. These effects have not been observed in preclinical studies to date. Other marketed IgG4 humanized monoclonal antibodies, such as natalizumab, eculizumab, and pembrolizumab, are used clinically. These antibodies largely target the immune system. There appears to be a spectrum of target-related negative effects when the total antibody dose gets too high. This pattern is not evident with TB006 in the preclinical toxicology profile, but will be closely observed in Phase 1 studies.

When performed correctly and according to industry standards, vein puncture complications are rare. However, even when properly performed, complications can include fainting, dizziness, hematoma, nerve injury, and arterial puncture or laceration. Blood draws and administration of the IV infusion in this study will be performed adequately trained medical staff to minimize any risk to patients.

Risks associated with a lumbar puncture can include headache, pain or numbness, bleeding, infection, puncture pain, and herniation. The lumbar puncture procedure will be performed by medical staff experienced with the technique and using aseptic techniques to minimize any risk to patients.

MR images are performed without the use of ionizing radiation, so patients are not exposed to the harmful effects of ionizing radiation. AEs for MRI scans are very rare. Millions of MRI scans are performed in the US every year, and the FDA receives around 300 AE reports for MRI scanners and coils each year from manufacturers, distributors, user facilities, and patients. The majority of these reports describe heating and/or burns (thermal injuries). To produce good quality images, patients must generally remain very still throughout the entire MRI procedure. The MRI procedure was be performed by medical staff experienced with the equipment and the technique to minimize any risk to patients.

The safety assessments to be performed in this study included clinical chemistry, hematology and urinalysis analyses, vital signs, ECGs, physical and neurological examinations, and assessments of AEs, SAEs, and C-SSRS are standard evaluations to ensure patient safety in this clinical study.

Benefit Assessment: TB006 is a humanized IgG4 (S228P) type monoclonal antibody that is highly specific and has high affinity to human Gal-3. It has demonstrated efficacy in preclinical models of neurodegenerative disorders such as AD and traumatic brain injury, and results are consistent with or superior to other compounds that have advanced into late-stage clinical studies. Thus, there is potential for TB006 to be effective in these disorders. The preclinical safety profile of TB006 supports the further investigation of TB006 in clinical studies.

Understanding that this therapy may have no clinical benefits to patients themselves, patients in this study contributed to the process of developing new therapies in an area of unmet need.

Patients underwent clinical evaluations/assessments associated with study procedures (e.g., safety laboratory tests, vital sign and ECG measurements, and physical and neurological examinations), which would be a potential benefit for their own health awareness.

During confinement in the clinic, meals and access to forms of entertainment were provided to patients. Patient expenses for time, travel, and other costs were offset by a monetary compensation, consistent with standards for such study participations and after approval from the IRB to ensure that no coercion or undue influence are imposed on patients.

Overall Benefit Risk Conclusion: The above benefit risk assessment supports the conduct of this clinical study.

The preclinical data on TB006 to date suggests a wide margin of safety and minimal risk of adverse effects. The risks associated with study procedures, including lumbar puncture and blood draws, are also very low. Any potential adverse effects associated with the study drug and procedures are further minimized by intense safety monitoring and medical oversight by the investigator and study staff. Therefore, the potential risks identified in association with TB006 are justified by the anticipated benefits that may be afforded to patients with AD, if TB006 is successfully developed.

This study was the second clinical study conducted with TB006. This study was conducted in parallel and combined with the Phase 1 SAD study (Protocol number TB006HV1101). Each group in this study commenced after the safety, tolerability, and available PK of the single dose equivalent to the combined total of 5 qw doses planned in this study has been established.

This study characterized TB006 safety, tolerability, plasma and CSF PK, and efficacy. In addition, the effect of TB006 on CSF and plasma biomarkers Aβ40, tau (phosphorylated), NFL, NFH, and Gal-3, as well as cognition, using the Clinical Dementia Rating Scale, the MMSE, the Cognitive Drug Research system battery, and the NPI, was evaluated. Brain atrophy measured by volumetric measurements as well amyloid plaque formation was assessed through MRI and PET.

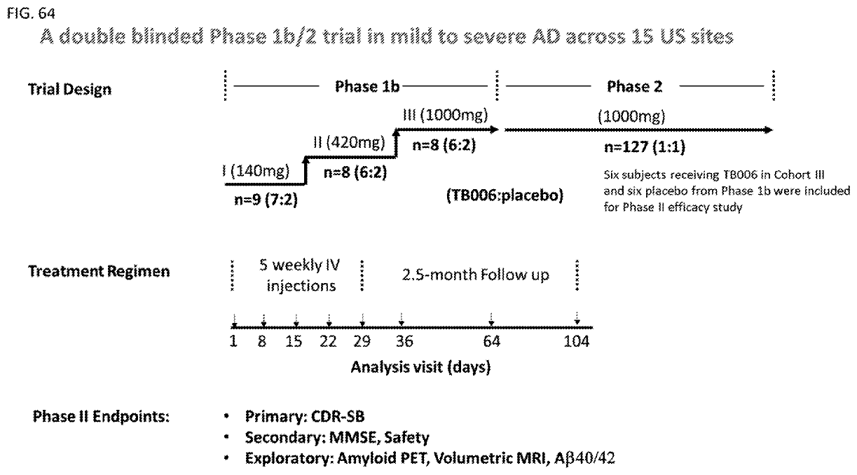

FIG. 58, FIG. 64A, and FIG. 64B, are charts depicting study design and treatment schema for parts 1 and 2 of a two part study of the safety efficacy of anti-Gal3 antibodies.

FIG. 59 is a chart depicting some embodiments of study design and treatment schema interleaving parts 1 and 2 of a two part study of the safety efficacy of anti-Gal3 antibodies.

Study Design: This was a seamless, two-part Phase 1b/Phase 2a, multi-center, randomized, double-blind, placebo-controlled study. Part 1 was a-MAD escalation study to evaluate the safety, tolerability, PK, PD, and efficacy of TB006 in patients with AD. Part 1 of this study assessed the safety and tolerability of three TB006 dose levels. The highest tolerated dose level from Part 1 was used in Part 2 of the study. Part 2 assessed the efficacy of TB006 on cognition using the Clinical Dementia Rating scale-Sum of Boxes (CDR-SB) as the primary endpoint. Secondary efficacy endpoints included the Mini Mental Status Examination (MMSE), the Cognitive Drug Research (CDR) System, the Neuropsychiatric Inventory (NPI), and imaging (magnetic resonance imaging [MRI]/positron emission tomography [PET]) and fluid (Gal-3 and Aβ42) biomarkers. A total of 5 doses were administered intravenously (IV) over 1 hour at weekly intervals to all patients. Dosing duration was limited to one month due to available toxicology coverage at the time. Part 1 patients who received the active 1000 mg dose, all Part 1 patients who received placebo, and all Part 2 patients were included in the efficacy analysis set (EAS). Randomization was done centrally via an Interactive Voice Response System (IVRS) and was stratified according to patient severity (baseline MMSE≥20-24; <20). Part 2 was a multi-center, randomized, double-blind, placebo-controlled, parallel-group study to evaluate the efficacy of TB006 after 1 month of treatment. The total study duration for each patient was up to approximately 19 weeks.

Table 7 depicts characteristics of the unblinded participants in the efficacy study at baseline.

TABLE 7

| | TB006 | | Placebo | | Total | |
|---|---|---|---|---|---|---|
| Variable | (N = 67) | | (N = 73) | | (N = 140) | |
| Female-n (%) | 33 | (49.3) | 41 | (57.5) | 75 | (53.6) |
| Age--yr-n | 73 | (50-90) | 71 | (56-88) | 72.4 | (50-90) |
| (range) | | | | | | |
| Race or ethnic | | | | | | |
| group-n (%) | | | | | | |
| Asian | 1 | (1.5) | 0 | | 1 | (0.7) |
| Black | 4 | (6.0) | 7 | (9.6) | 11 | (7.8) |
| White | 61 | (91.0) | 66 | (90.4) | 127 | (90.7) |
| Hispanic ethnic | 31 | (46.3) | 41 | (56.2) | 72 | (51.4) |
| group | | | | | | |
| Other | 1 | (1.5) | 0 | | 1 | (0.7) |
| CDR-SB-mean | 6.1 +/− 3.3 | | 5.7 +/− 3.5 | | 5.87 | (1-16) |
| (range) | (1-16) | | (0.5-15) | | | |
| MMSE-mean | 17.6 | (5-24) | 18 | (2-24) | 17.85 | (2-24) |
| (range) | | | | | | |
| MMSE 21-24-n | 21 | (31.3) | 26 | (35.6) | 47 | |
| (%) | | | | | | |
| MMSE <=20-n | 46 | (68.7) | 47 | (64.4) | 93 | |
| (%) | | | | | | |
| Plaque positive-n | 19 | (28.4) | 26 | (35.6) | 45 | |
| (%) | | | | | | |
| Plaque negative- | 48 | (71.6) | 47 | (64.4) | 95 | |
| n (%) | | | | | | |

Of the 140 total study participants, 67 received TB006 and 73 received a placebo. Of the 140 total participants, 74 (53.6%) were females and 65 (46.4%) were males between 50-90 years old with an average age of 72.4 years. Of those participants receiving TB006, 33 (49.3%) were females and 34 (50.7%) were males between 50-90 years old with an average age of 73 years. Of those participants receiving placebo, 42 (57.5%) were females and 31 (42.5%) were males between 56-88 years old with an average age of 71 years.

Of the 140 total participants, 7 (9.6%) were black, 25 (34.2%) were white, 71 (50.7%) were Hispanic, and 1 (0.7%) was of another racial or ethnic background. Of those participants receiving TB006, 1 (1.5%) was Asian, 4 (6%) were black, 30 (44.8%) were white, 41 (56.2%) were Hispanic, and 1 (1.5%) was of another racial or ethnic background. Of those participants receiving placebo, 1 (1.5%) was Asian, 4 (6%) were black, 30 (44.8%) were white, and 31 (46.3%) were Hispanic.

Of the 140 total participants, the baseline CDR-SB score ranged from 0.5-16 with a mean score of 5.7. Of those participants receiving TB006, the baseline CDR-SB score ranged from 1-16 with a mean score of 6.1. Of those participants receiving placebo, the baseline CDR-SB score ranged from 0.5-15 with a mean score of 5.7.

Of the 140 total participants, the baseline MMSE ranged from 2-24 with a mean MMSE of 17.85. Of all participants, 47 (33.6%) had an MMSE between 21 and 24. Of those participants receiving TB006, the baseline MMSE ranged from 5-24 with a mean MMSE of 17.6. Of those participants receiving TB006, 21 (31.3%) had an MMSE between 21 and 24. Of those participants receiving placebo, the baseline MMSE ranged from 2-24 with a mean MMSE of 18. Of those participants receiving placebo, 26 (35.6%) had a baseline MMSE between 21 and 24.

Of the 140 total participants, 45 (32.1%) were plaque positive. Of those participants receiving TB006, 19 (28.4%) were plaque positive. Of those participants receiving placebo, 26 (35.6%) were plaque positive.

Patients received a total of 5 doses, given in qw intervals. In Part 1, the planned starting dose in Group 1 was 140 mg qw for 5 weeks. Subsequent planned doses were 420 and 1,000 mg qw for Groups 2 and 3, respectively. The planned dose in Part 2 was the highest safe and well-tolerated dose from Part 1. All doses were infused IV over 1 hour. Part 1 was interleaved with the SAD study (Study TB006HV1101). Each group in Part 1 was commenced after the safety, tolerability, and PK of the single dose equivalent to the combined total of 5 qw doses planned in this study had been established (minimum of 14 days from last patient dosing). Explicitly, dosing in Group 1 (140 mg qw) commenced when the safety and tolerability of the 700 mg single dose in the SAD study had been established. Groups 2 (420 mg qw) and 3 (1,000 mg qw) began after a review of the available safety, tolerability and PK data from the previous group; and after the 2,100 and 5,000 mg single dose, respectively, had been administered. Dose levels may have been adjusted depending on the safety, tolerability, and PK of doses in the SAD study and previous groups in this study. Patients were enrolled across all active centers into each dose group sequentially.

In Part 1, 8 patients were enrolled into each dose group; 6 patients in each group were randomized to active TB006 treatment, and 2 patients to placebo.

Dose escalation decisions used safety, tolerability and available PK data through the first dose administered to the last patient in each group and a minimum of 14 days from the respective dose group in the SAD study (Protocol number TB006HV1101). Data was reviewed by a blinded SRC.

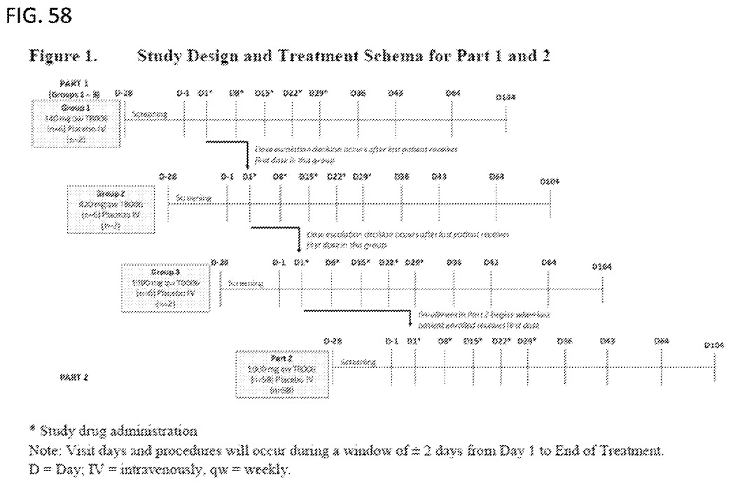

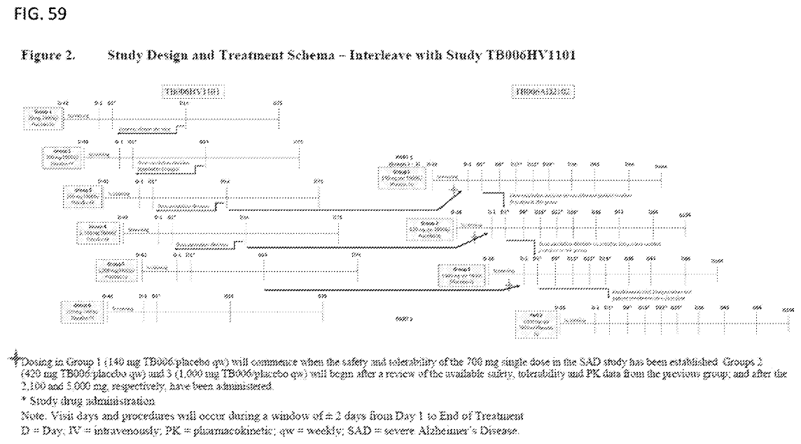

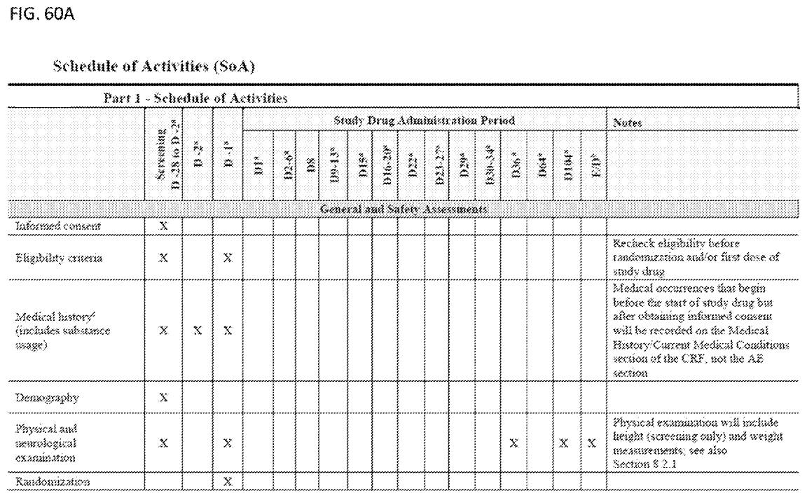

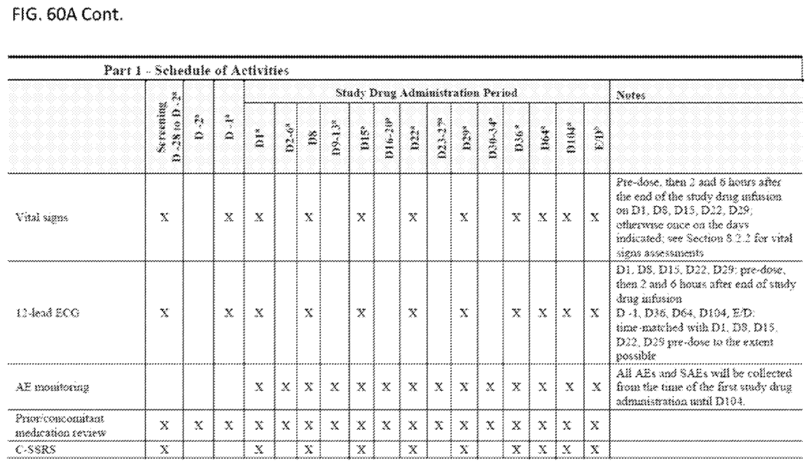

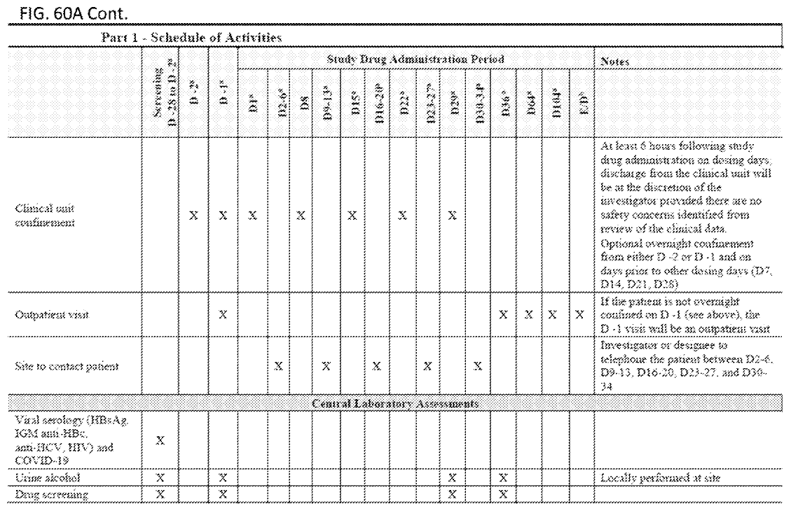

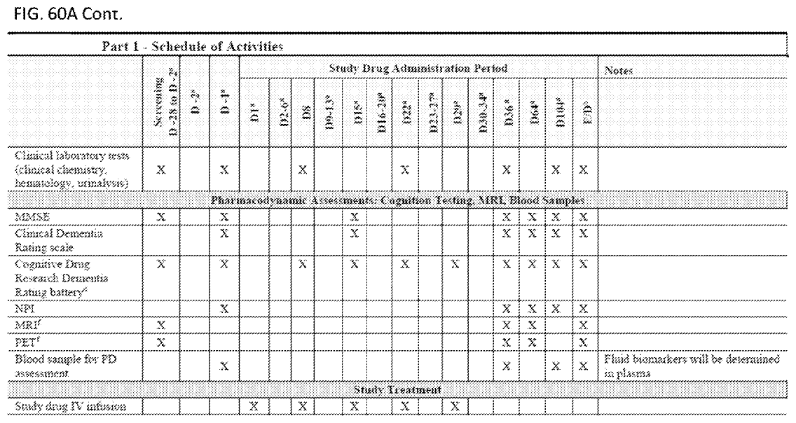

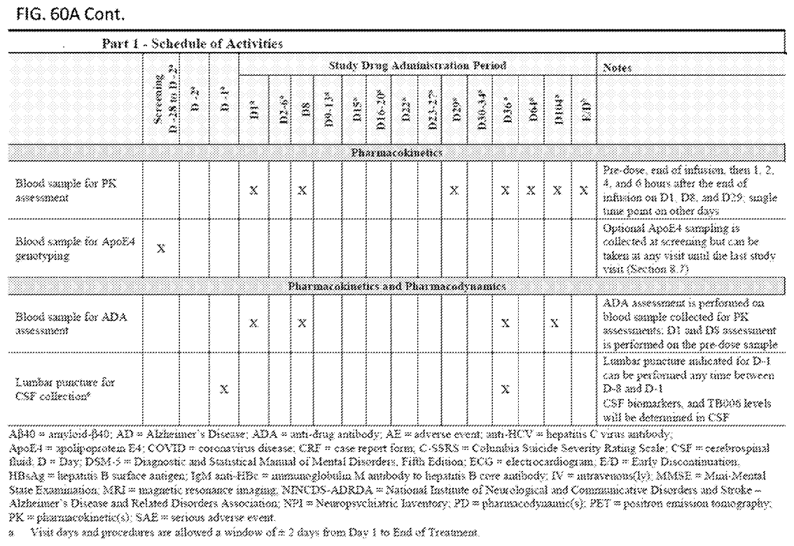

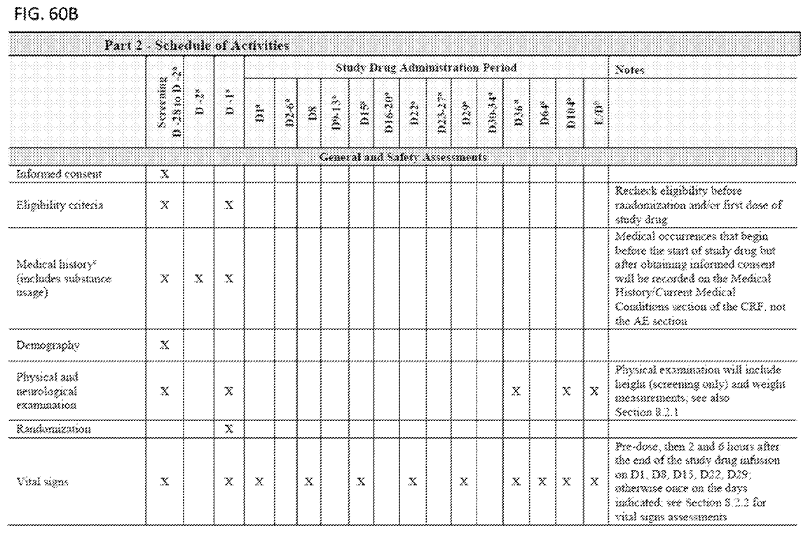

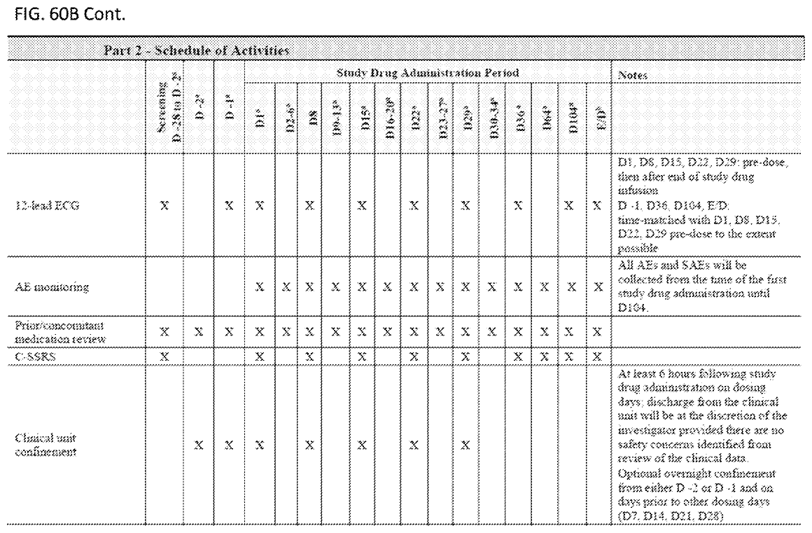

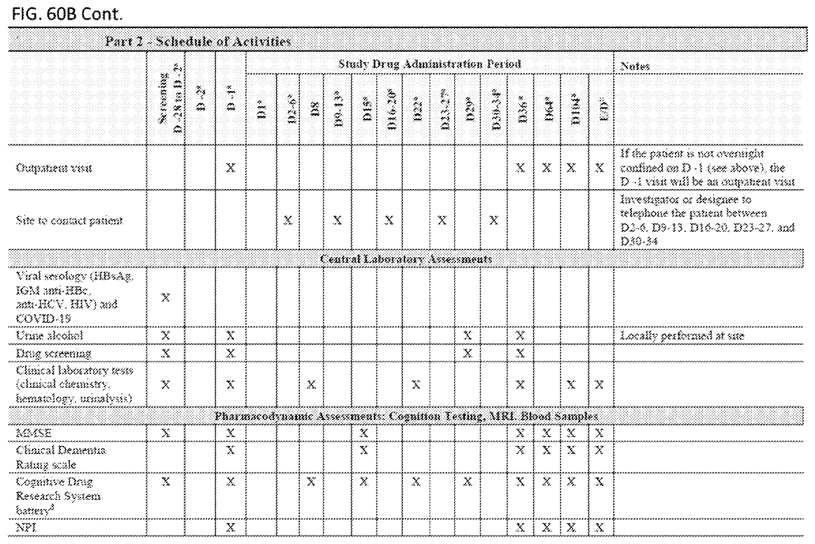

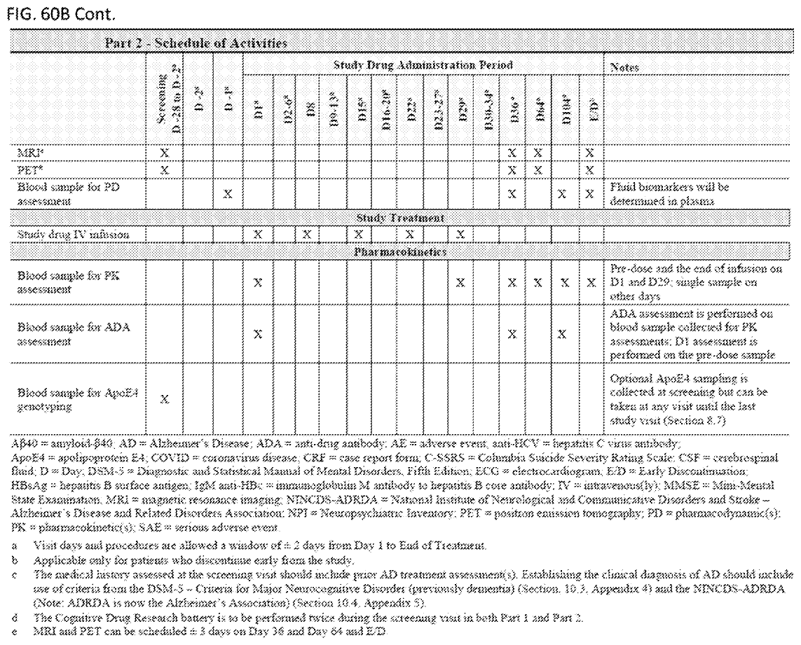

Part 2 commenced after the last patient in Part 1 had received their first dose. The planned dose in Part 2 was 1,000 mg qw×5 doses, but may have been adjusted depending on the safety and tolerability of the doses in Part 1. Patients were randomized to active TB006 or placebo (1:1) and followed the same dosing schedule and procedures (with some exceptions-see Part 2 SoA (FIG. 60) as in Part 1. Randomization in Part 2 was stratified according to baseline patient severity, with mild AD patients (MMSE 21-24) and moderate-severe AD patients (MMSE≤20) in each strata.

This was an outpatient study. Patients returned to the clinic for screening and baseline procedures, on dosing days, and on follow-up visits. Patients were given the option of checking into the clinic and staying overnight on Day-2 or Day-1, as well as on days prior to the other dosing days.

Assessments for safety and tolerability, efficacy, PK, and PD were performed throughout the study. If a patient reported any AEs, they may have been required to return to the clinical unit at the discretion of the investigator for additional assessments. All AEs were followed to adequate resolution.

Disclosure Statement: This was a randomized, placebo-controlled, sequential and parallel group treatment study that was patient and investigator blinded.

Number of Participants: In Part 1, 8 patients were enrolled into each dose group, thus a total of approximately 24 patients were randomized in this portion of the study. In Part 2, 116 patients were enrolled, 58 into active TB006 treatment and 58 to placebo.

Dose Groups and Duration: In Part 1, three dose groups were planned, each representing one dose level of TB006 or placebo. In Part 2, two groups (active TB006 and placebo) were planned.

Justification for Dose: The selection of doses in the SAD study was made after a careful assessment of the preclinical pharmacology and toxicology data while allowing for cautious escalation to the highest safe exposures or the maximum tolerated dose (FDA, 2005). The doses planned for this study were tentative and may have been adjusted based on safety, tolerability, and available PK data from the SAD study and the previous group(s) in this study. The intent was to maintain the safety margins as described here.

The preclinical safety of TB006 was explored in a definitive 1 month GLP toxicology study in cynomolgus monkeys. Doses used in this study were 10, 30, 100, and 200 mg/kg, administered qw for 5 doses. Mild lymphoid hyperplasia occurred in males at the 10, 100, and 200 mg/kg dose levels, but based on mild severity, lack of dose dependency and historical test facility data from similar studies in cynomolgus monkeys, this finding was considered a species specific mild, reactive immune response and not an adverse toxicologic finding. No other adverse effects occurred, and so the NOAEL in cynomolgus monkeys dosed once qw for 4 weeks was 200 mg/kg. The mean Day 29 exposures associated with this dose were Cmax and AUCO-τ of ~13,000 μg/mL and ~135,000 h*ug/mL, respectively.

Using a body surface area calculation and a 10×safety factor, the maximum recommended safe starting dose would be 6.4 mg/kg, ~440 mg in a 70 kg person in the SAD study. Consideration, however, was given to preclinical efficacy data, where activity was demonstrated at a dose level of 10 mg/kg in mice. The HED conversion of this dose was approximately 70 mg in a 70 kg person. Subsequent planned doses in the SAD study were 200, 700, 2,100 and 5,000 mg. At the highest dose, a predicted safety margin from the NOAEL was estimated to be 8-fold on Cmax and a 6-fold margin on AUCO-τ based on early PK projections. Planned doses are to be modified based on safety and tolerability and PK (to retain these margins).

If TB006 behaves like other IgG4 antibodies, the half-life will likely be longer than the approximate 10 to 12 days observed in monkeys. Qw dosing will result in accumulation. Despite this, the safety and tolerability of a single dose is a conservative approach to estimating the safety and tolerability of the same dose divided into qw administration for 1 month. In addition, a similar conservative approach can be applied to establishing initial safety margin estimates by using one compartment simulations derived from monkey toxicokinetic data. The highest proposed dose of 1,000 mg qw (total regimen=5,000 mg) affords a safety margin of >8-fold on the Cmax and 6-fold on the AUC from the NOAEL in the 1 month toxicology study. The lowest dose of 140 mg qw (total regimen=700 mg) results in a safety margin of nearly 60- and 30-fold on the Cmax and AUC, respectively. Using the HED conversion from the 200 mg/kg dose level in monkeys, the margins from the NOAEL range from ~30-fold at the lowest dose to ~5-fold at the highest dose for a 70 kg person.

The mid dose of 420 mg qw enables a cautious escalation between doses. Evidence of intolerance or other safety signals, or PK differences that result in reductions in safety margins from the SAD study or previous doses in this study, may necessitate a change in planned doses. While TB006 is a first in class anti-Gal-3 antibody, a wider array of approved IgG4 human monoclonal antibodies have been used clinically. Most of these target the immune system, and there appears to be a spectrum of negative effects, largely immune-mediated, when the total antibody dose gets too high. These and all other toxicities will be closely monitored. Dose escalation will either be modified or will cease when evidence of toxicity appears.

The chosen dose range covers a potentially efficacious dose to a projected supratherapeutic dose. Successful safety, tolerability and PK characterization of a wide dose range will enable efficient drug development. Traditionally, MAD studies employ the planned clinical dosing interval, and patients are dosed to steady state to determine steady state PK and safety. The projected half-life of TB006 is expected to enable monthly dosing in later clinical studies. The qw dosing schedule used in this study will enable a determination of multiple dose PK and provide an estimate of repeat dose safety and tolerability.

Each patient was screened for eligibility up to 28 days prior to first dosing, and study assessments were performed up to 104 days after first dosing. The total study duration for each patient was therefore be up to approximately 19 weeks.

Data Monitoring/Other Committee: In Part 1 only.

End of Study (Individual Patient): A patient was considered to have completed the study if he/she had completed all study visits through the Day 104 assessments as shown in the SoA (FIG. 60).

End of Study (End of Trial): The end of the study was defined as the date of the last visit of the last patient in the study or last scheduled procedure shown in the SoA (FIG. 60) for the last patient in the study.

Study Inclusion Criteria: Patients were eligible to be included in the study only if all of the following criteria applied:
1.Male and/or female>50 years of age at the time of signing the informed consent.
2.Body weight of ≥50 kg and BMI between 18 and 35 kg/m2, inclusive.
3.MMSE score of 24 or less.
4.Must be ambulatory.
5.Clinical diagnosis of AD consistent with the following:
a. Probable AD, according to NINCDS-ADRDA (Section 10.5 [Appendix 5])
b. Meets the DSM 5-Criteria for Major Neurocognitive Disorder (previously dementia) (Section 10.4 [Appendix 4])
6.Contraceptive use by men should have been consistent with local regulations regarding the methods of contraception for those participating in clinical studies.
   a. Male patients:
male patients were eligible to participate if they agreed to the following from the first day of dosing through 6 months following the last dose of study drug: refrain from donating sperm PLUS, either: be abstinent from heterosexual intercourse as their preferred and usual lifestyle (abstinent on a long term and persistent basis) and agree to remain abstinent unless partner is not a WOCBP, OR, must have agreed to use contraception/barrier as detailed below:
   Agreed to use a male condom and female partner use of an additional highly effective contraceptive method with a failure rate of <1% per year when having sexual intercourse with a WOCBP who was not currently pregnant.
   b. Females must have been of non-childbearing potential defined as: at least 12 months post-menopausal; surgically sterile, resulting from but not limited to tubal ligation, hysterectomy, and oophorectomy. Documented infertility from other causes, such as endometriosis or uterine fibroids. The investigator was responsible for review of medical history, menstrual history, and recent sexual activity to decrease the risk for inclusion of a woman with an early undetected pregnancy. Patients or caregiver has the ability to understand the purpose and risks of the study and provide signed and dated informed consent, which includes compliance with the requirements and restrictions listed in the ICF and in this protocol. Patients whose caregiver signs the informed consent must provide their assent; may or may not be receiving 1 or more of the currently marketed cognition enhancing AD medications, including, but not limited to donepezil, memantine, rivastigmine, and galantamine. If receiving, it must be the same medication and dose the past 2 months prior to screening. Either currently or previously (in pre AD condition) literate and capable of reading, writing, and communicating effectively with others.

Patients with different comorbidities like diabetes, hypertension were eligible and included

Study Exclusion Criteria: Patients are excluded from the study if any of the following criteria apply:
1. Any medical or neurological condition other than AD that in the opinion of the investigator could be a contributing cause of the patient's dementia (e.g., medication use, vitamin B12 deficiency, abnormal thyroid function, stroke or other cerebrovascular condition, diffuse Lewy body disease, head trauma).
2. History within the past 6 months or evidence of clinically significant psychiatric illness (e.g., major depression, schizophrenia, or bipolar affective disorder). Diagnosis of a dementia-related CNS disease other than AD (e.g., Parkinson's Disease, Huntington's Disease, frontotemporal dementia, multi-infarct dementia, dementia with Lewy bodies, normal pressure hydrocephalus).
3. Identification of other known cause of dementia or any other clinically significant contributing co-morbid pathologies at screening MRI, in the opinion of the investigator.
4. Participation in any other drug, biologic, device, or clinical study or treatment with any investigational drug or approved therapy for investigational use within 30 days (or 5 half lives, whichever is longer) prior to screening, and/or participation in any other clinical study involving experimental medications for AD within the 60 days (or 5 half lives, whichever is longer) prior to screening.
5. Any contraindications to having a brain MRI e.g., pacemaker; non-MRI-compatible aneurysm clips, artificial heart valves, or other metal foreign body; claustrophobia).
6. Any untreated or unstable clinically significant medical condition (i.e., hypertension, diabetes, chronic obstructive pulmonary disorder, asthma, depression, etc.) as judged by the investigator.
7. Any clinically significant findings in medical examination, including physical examination, 12-lead ECG, clinical laboratory tests (specifically, ALT>1.5×ULN, bilirubin>1.5×ULN [bilirubin>1.5×ULN is acceptable if bilirubin is fractionated and direct bilirubin<35%], QTcB>450 msec for male patients or >470 msec for female patients). Undergone major surgery≤2 months before study drug administration.
8. Abnormalities in lumbar spine previously known or determined by a screening lumbar X-ray (if conducted). History of clinically significant back pain, back pathology, and/or back injury (e.g., degenerative disease, spinal deformity, or spinal surgery) that may predispose patient to complications or technical difficulty with lumbar puncture.
9. Evidence or history of significant active bleeding or coagulation disorder or use of non-steroidal anti-inflammatory drugs or other drugs that affect coagulation or platelet function within 14 days prior to lumbar catheter insertion.
10. Allergy to lidocaine (Xylocaine®) or its derivatives. Medical or surgical conditions for which lumbar puncture is contraindicated.
11. Loss of more than 100 mL blood (e.g., a blood donation) within 2 months before first study drug administration, or has received any blood, plasma, or platelet transfusions within 3 months before Day 1, or plans to donate blood during the study or within 3 months after the study.
12. Recent (1-month) history of a positive COVID-19 test result or disease symptoms of COVID-19 disease such as shortness of breath, cough, rhinorrhea, and sore throat, etc.
13. Known history of, or a positive test result for, HBsAg, IgM anti HBc, anti HCV, or HIV types 1 or 2 at screening. Patients with a documented history of treatment for Hepatitis C are otherwise eligible to participate.
14. Regular alcohol consumption within 6 months prior to the study defined as: an average weekly intake of >20 units for males or >16 units for females. One unit is equivalent to 8 g of alcohol: a half pint (~240 mL) of beer, 1 glass (125 mL) of wine or 1 (25 mL) measure of spirits.
15. Meets DSM-5 criteria for moderate or severe substance use disorder within the past 12 months, or has a positive test for substances of abuse, or has used substances, including but not limited to opiates, methadone, buprenorphine, methamphetamine, cocaine, amphetamines recreationally within the past 12 months.
16. Unable to complete this study for other reasons or the investigator believes that he or she should be excluded.

### Lifestyle Considerations:

### Meals and Dietary Restrictions:

No fasting or other dietary restrictions, including caffeine, were applicable. Dietary restrictions may have applied to patients undergoing PET scans, depending on the requirements of the facility. These restrictions could have included reduced carbohydrate intake for up to 24 hours and fasting for 6 hours prior to the procedure.

### Alcohol:

Moderate alcohol consumption (up to 2 units/day [males] and 1 unit/day [females]) was allowed throughout the study. One unit is equivalent to 8 g of alcohol: a half pint (~240 mL) of beer, 1 glass (125 mL) of wine or 1 (25 mL) measure of spirits.

### Activity:

Patients abstained from strenuous exercise for 24 hours before each blood collection for clinical laboratory tests. Patients may have participated in light recreational activities during the study (e.g., moderate exercise such as walking or bicycling).

### Screen Failures:

Screen failures were defined as patients who consented to participate in the clinical study but were not subsequently randomized. A minimal set of screen failure information was required to ensure transparent reporting of screen failure patients to meet the CONSORT publishing requirements and to respond to queries from regulatory authorities. Minimal information includes demography, screen failure details, eligibility criteria, medical history, prior therapies, and any SAE.

Individuals who did not meet the criteria for participation in this study (screen failure) may have been rescreened if they have the potential to be included. Rescreened patients were assigned the same patient number as for the initial screening. Informed consent was required before patients could be rescreened.

Study Drug: Study drug as defined in this example refers to TB006 or placebo, intended to be administered to a study patient according to the study protocol.

Study Drug Administration: The study drug for this study was TB006 and the control drug was placebo.

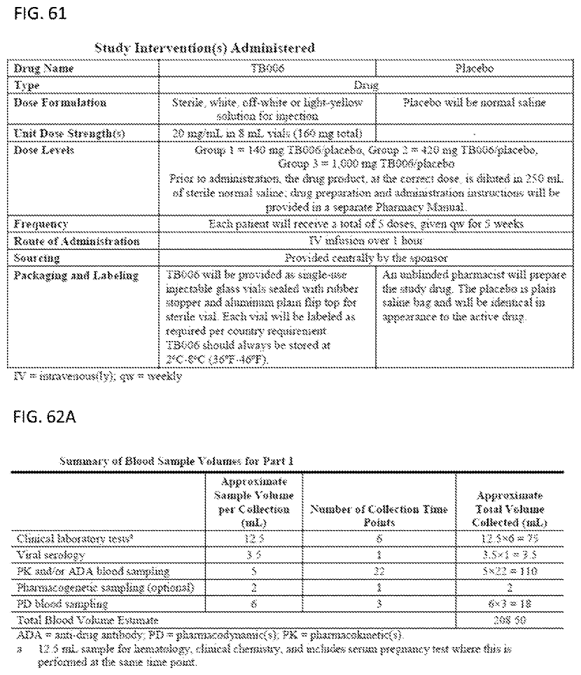

FIG. 61 is a table depicting study interventions administered in a study of the safety and efficacy of anti-Gal3 antibodies.

Study drug was administered as a 1 hour IV infusion, at planned dose levels as outlined in FIG. 61.

All planned dose levels may have been adjusted upon review of the available PK and safety data from the SAD and Part 1 of this study as referenced previously. Every effort was made to achieve the safety margins, discussed above, through the highest dose. In addition, safety, tolerability and available PK data from this study was taken into consideration for the second and third dose levels of Part 1, and the selected dose in Part 2. Lastly, dose groups in Part 1 may have been added or removed from the planned design based on emerging data.

Dose Escalation and Stopping Considerations: While concrete dose escalation stopping criteria was not included, the following variables (at a minimum) were taken into account by the SRC at the protocol-specified time points for these decisions, and when events arose during the course of study treatment: Number and frequency of treatment-related AEs characterized as moderate or severe in severity; number and frequency of treatment-related SAEs; discontinuation due to AEs; emergent cardiovascular events, such as QTc increases or vital sign changes; liver function enzyme increases.

Measures to Minimize Bias: Randomization and Blinding”

Randomization: All patients were centrally assigned to randomized study drug using an Interactive Voice Response System (IVRS). Before the study was initiated, the log in information and directions for the IVRS were provided to each site.

In Part 1, 8 patients were enrolled into each dose group; 6 patients in each group were randomized to active TB006 treatment, and 2 patients to placebo (with 3 dose groups planned, for a total of 18 patients receiving TB006 treatment at different dose levels, and a total of 6 patients receiving placebo). In Part 2, 116 patients were randomized to active TB006 or placebo (1:1) and followed the same dosing schedule and procedures (with some exceptions-see SoA) as in Part 1. Randomization in Part 2 was stratified according to baseline patient severity, with mild AD patients (MMSE 21-24) and moderate-severe AD patients (MMSE≤20) in each strata.

On Day 1, patients were assigned a unique number (randomization number) in ascending numerical order as patients are enrolled. Once a randomization number was assigned, it was not re-assigned. The randomization number encoded the patient's assignment to either active TB006 treatment or placebo, according to a randomization schedule generated prior to the study. Each patient received blinded study drug, labeled with his/her unique randomization number.

Blinding: In Part 1, in each dose group, 6 patients was randomized to active TB006 treatment, and 2 patients to placebo. In Part 2, 58 patients were randomized to active TB006 treatment, and 58 patients to placebo. Investigators remained blinded to each patient's assigned study drug throughout the course of the study. In order to maintain this blind, an unblinded pharmacist was responsible for the reconstitution and dispensation of all study drug. Active study drug and placebo were made to be identical in appearance.

In the event of a Quality Assurance audit, the auditor(s) allowed access to unblinded study drug records at the site(s) to verify that randomization/dispensing had been performed accurately.

The IVRS was programmed with blind-breaking instructions. In case of an emergency, the investigator had the sole responsibility for determining if unblinding of a patient's treatment assignment was warranted. Patient safety was always be the first consideration in making such a determination. If the investigator decided that unblinding was warranted, the investigator made every effort to contact the sponsor prior to unblinding a patient's treatment assignment unless this could have delayed emergency treatment of the patient. If a patient's treatment assignment was unblinded, the sponsor was notified within 24 hours after breaking the blind. The date and reason that the blind was broken was recorded in the source documentation and CRF, as applicable.

Study Assessments and Procedures: Study procedures and their timing are summarized in the SoA (FIG. 60). Protocol waivers or exemptions were not allowed. Procedures conducted as part of the patient's routine clinical management (e.g., blood count) and obtained before signing of the ICF may have been utilized for screening purposes, provided the procedures met the protocol-specified criteria and were performed within the time frame defined in the SoA (Table 1 and Table 2). The maximum amount of blood collected from each patient over the duration of the study, including any extra assessments that may be required, did not exceed 250 mL. Repeat or unscheduled samples may have been taken for safety reasons or for technical issues with the samples.

### General Study Periods:

Screening, Enrollment and/or Randomization:

Screening occurred between Day-42 and Day-2. The purpose of the screening period was to obtain informed consent and to establish protocol eligibility. Informed consent was obtained after the study had been fully explained to each patient and before the conduct of any screening procedures or assessments.

Screening assessments were performed as outlined in the SoA (FIG. 60).

Following randomization, patients received study drug as an IV infusion at qw intervals, for 5 doses. Study assessments were performed up to Day 104 as outlined in the SoA (FIG. 60).

Safety Assessments: Planned time points for all safety assessments are provided in the SoA (FIG. 60).

Physical and Neurological Examinations: A complete physical examination included, at a minimum, assessments of the cardiovascular, respiratory, gastrointestinal and neurological systems. The neurological examination assessed mental status, motor and sensory skills, hearing and speech, vision, coordination, and balance. Height and weight were also be measured and recorded. Height was only be measured at screening.

Vital Signs: Oral temperature, pulse rate, respiratory rate, and blood pressure were assessed as indicated in the SoA (FIG. 60). Blood pressure and pulse measurements were assessed with a completely automated device. Manual techniques were used only if an automated device was not available. Blood pressure and pulse measurements were preceded by at least 5 minutes of rest with the patient in a quiet setting without distractions (e.g., television, cell phones). Blood pressure measurements were taken with the patient in the sitting position. Vital signs were taken before blood collection for laboratory tests, if that was scheduled for the same time point.

Electrocardiograms: 12-lead ECG measurements were obtained as outlined in the SoA (FIG. 60) using an ECG machine that automatically calculated the heart rate and measures PR, QRS, QT, and QTc intervals (triplicate measurements will be obtained at all-time points, except at the screening visit where a single measurement was obtained). At each time point at which triplicate ECG were required, 3 individual ECG tracings were obtained at 2-minute intervals. On non-dosing days, effort was made to perform 12-lead ECGs time-matched with the pre-dose time point on dosing days. The PI or designated site physician reviewed and assessed all ECGs as Normal, Abnormal, or Not Evaluable. Once signed, the original ECG tracing was retained with the patient's source documents.

In Part 1 only, ECGs were transferred electronically to an ECG central reader for analysis according to the study-specific recommendations included in the ECG manual. The central ECG reader review machine read and provided corrected values as specified in the manual. The following parameters were recorded: PR interval, RR interval, QRS complex/duration, QT interval, QTc, heart rate, and clinical interpretation. The results from the central reader were captured in the study database, together with the investigator assessment of the ECG.

FIG. 63 is a table depicting protocol required clinical laboratory safety assessments.

Clinical Safety Laboratory Assessments: The tests detailed in FIG. 63 were performed by the central laboratory; see the SoA (FIG. 60) for the timing and frequency. Additional tests may have been performed at any time during the study as determined necessary by the investigator or required by local regulations. Clinically significant abnormal laboratory findings were those that were not associated with the underlying disease, unless judged by the investigator to be more severe than expected for the patient's condition. All laboratory tests with values considered clinically significantly abnormal during participation in the study were repeated at appropriate intervals until the values returned to normal or baseline or were no longer considered clinically significant by the investigator or Medical/Scientific Monitor. If such values did not return to normal/baseline within a period of time judged reasonable by the investigator, the etiology was identified and the sponsor notified. All protocol-required laboratory assessments, were conducted in accordance with the laboratory manual and the SoA (FIG. 60).

Pharmacokinetics: Whole blood samples of approximately 5 mL were collected for measurement of plasma concentrations of TB006 as specified in the SoA (FIG. 60). Additional samples may have been collected at additional time points during the study if warranted and agreed upon between the investigator and the sponsor. The timing of sampling may have been altered during the course of the study based on newly available data (e.g., to obtain data closer to the time of peak plasma concentrations) to ensure appropriate monitoring. Samples were used to evaluate the PK of TB006. Each plasma sample was divided into 2 aliquots (1 each for PK and a back-up). Samples collected for analyses of plasmaTB006 concentration may also have been used to evaluate safety or efficacy aspects related to concerns arising during or after the study. Other than ApoE4 genetic testing from a sample at the Screening visit, genetic analyses was not performed on these plasma samples. Patient confidentiality was maintained.

Lumbar puncture procedure was performed at the time points as specified in the SoA (Part 1 only) (FIG. 60) to obtain CSF samples for measurement of concentrations of TB006. The lumbar procedure was optional for participating patients.

### Pharmacodynamics:

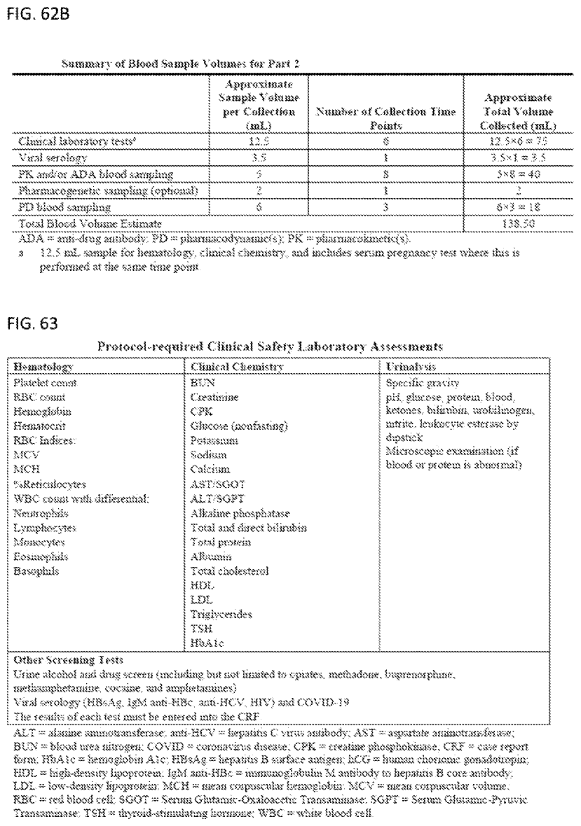

FIG. 62 depicts blood samples and volumes drawn from subject's in a two part study of the safety efficacy of anti-Gal3 antibodies. FIG. 62A is a table depicting blood samples and volumes drawn from subject's in part one of a two part study of the safety efficacy of anti-Gal3 antibodies. FIG. 62B is a table depicting blood samples and volumes drawn from subject's in part two of a two part study of the safety efficacy of anti-Gal3 antibodies.

Blood Samples for Biomarker Assessments: Venous blood samples of approximately 5 mL will be collected for measurement of plasma Aβ40, tau (phosphorylated), NFL, NFH, Gal-3, and other relevant biomarkers at the time points specified in the SoA (FIG. 60).

### Lumbar Puncture (Part 1 Only)

Lumbar puncture procedure (optional for participating patients) was performed at the time points as specified in the SoA (FIG. 60) to obtain CSF samples for measurement of Aβ40, tau (phosphorylated), NFL, NFH, Gal-3, and other relevant biomarkers.

Cognition Testing: Patients performed a battery of cognition tests, including the MMSE, the Clinical Dementia Rating Scale, the Cognitive Drug Research system battery, and the NPI throughout the study to assess cognition. Tests, with the exception of the Cognitive Drug Research system battery were performed by paper only. The Cognitive Drug Research system battery was performed using an electronic device.

Mini-mental State Examination: The MMSE was administered at the time points indicated in the SoA (FIG. 60). The MMSE was administered by a trained member of the investigational team. At each site, the same individual, whenever possible, performed the MMSE evaluation on a specific patient throughout the study. The screening MMSE was used for purposes of determining patient eligibility and did not need to be reconciled with the baseline MMSE score.

Cognitive Drug Research System: The Cognitive Drug Research system was performed at the time points listed in the SoA (FIG. 60). Two training sessions were conducted at least 30 minutes apart during the screening period to allow patients to become familiar with the procedure, reduce test anxiety, reduce learning effects, and produce a stable baseline assessment.

Neuropsychiatric Inventory: The NPI was administered at the time points indicated in the SoA (FIG. 60). The NPI was administered by an independent, trained, and certified member of the investigational team. At each site, the same individual, whenever possible, performed the NPI evaluation on a specific patient throughout the study.

Magnetic Resonance Imaging: MRI was administered at the time points indicated in the SoA (FIG. 60). The procedure was performed at a center associated with the site. The MRI was scheduled after other screening procedures had been completed to ensure a high likelihood of patient eligibility.

During the MRI, patients were instructed to lie still in an enclosed environment for the duration, which was usually approximately 20 minutes. Patients were informed that they may hear a buzzing sound throughout the procedure and may hear an occasional thumping sound. Otherwise, the procedure was noninvasive and painless. After the procedure, patients may have needed a few minutes to reorient themselves in a sitting position.

Positron Emission Tomography: A PET scan was performed at the same time points as the MRI, as indicated in the SoA (FIG. 60). Similar to MRI, the PET scan was scheduled after other screening procedures had been completed to ensure a high likelihood of patient eligibility. Patients undergoing PET scans may have been asked to fast for 6 hours prior to the procedure, depending on the requirements of the facility. They may also have been asked to reduce carbohydrate intake for 24 hours prior to the procedure. A central reader may have been used in this study to interpret MRI and PET changes in amyloid deposits, volume, and other important clinical endpoints.

Pharmacogenetics: A 2 ml sample of blood was taken for ApoE4 genotyping at screening from patients who give separate written informed consent for this optional research. If this sample was not taken at screening, it may have been taken at any visit until the last study visit.

Immunogenicity Assessments: Antibodies to TB006 were evaluated in plasma samples collected from all patients at the time points according to the SoA (FIG. 60). These samples were tested by the sponsor or sponsor's designee. Anti-TB006 antibodies were determined on plasma samples collected for plasma PK determinations, and no additional blood samples needed to be obtained. Plasma samples were screened for antibodies binding to TB006 and the titer of confirmed positive samples was reported. Other analyses may have been performed to verify the stability of antibodies to TB006 and/or further characterize the immunogenicity of TB006. The detection and characterization of antibodies to TB006 was performed using a validated assay method by or under the supervision of the sponsor. All samples collected for detection of antibodies to study drug will also be evaluated for TB006 plasma concentration to enable interpretation of the antibody data. Antibodies may have been further characterized and/or evaluated for their ability to neutralize the activity of the study drug(s).

Statistical Hypotheses: The primary endpoint in Part 2 was the change from baseline Clinical Dementia Rating Scale-Sum of Boxes score at through Day 104. The primary null hypothesis to be tested was as follows: H0: There is no difference between TB006 group and placebo group in change from baseline Clinical Dementia Rating Scale-Sum of Boxes score at through Day 104.

Sample Size Determination: In Part 1, patients were recruited sequentially in separate increasing dose groups, with each group randomizing 6 patients to a specific dose of TB006 and 2 randomized to placebo. If all 3 planned dose levels were investigated, the study recruited approximately 24 patients in total (18 receiving TB006 treatment at the different dose levels, and 6 receiving placebo).

In Part 2, patients were randomized to TB006 or placebo in a 1:1 ratio so that approximately 58 patients were randomized to each group. Randomization in Part 2 was stratified according to baseline patient severity, with mild AD patients (MMSE 21-24) and moderate-severe AD patients (MMSE≤20) in each strata. Patients enrolled at the same TB006 dose level in Part 1, as well as all placebo patients, were included in the efficacy analyses in Part 2. Thus, the number of patients in each group for the primary efficacy analysis was approximately 64.

Assuming that the standard deviation of change from baseline Clinical Dementia Rating Scale-Sum of Boxes at Day 104 is 0.5, a total of 128 patients provided 80% power to detect a mean change from baseline difference of 0.25 at Day 104 using a 2-sided, 2 sample t-test at the 5% level of significance.

Populations for Analyses: For the purposes of analysis, the following analysis sets are defined:

Full analysis set (FAS): All patients who are randomly assigned to study drug.

Safety analysis set (SAS): All patients randomly assigned to study drug and who take at least 1 dose of study drug. Patients will be analyzed according to the study drug they actually received.

PK analysis set (PKS): All patients who received at least 1 dose of TB006 and have at least 1 post-dose blood sample with measurable TB006 concentrations.

PD analysis set (PDS): All patients who received blinded study drug and have at least 1 post-dose evaluable PD assessment.

The FAS was used for the analysis of disposition and protocol deviations. The SAS was used for all other analyses, except specifically for PK and PD. The FAS and the SAS was the same if all randomized patients were dosed with study drug. The PKS was used for all PK analyses and the PDS was used for all PD and efficacy analyses.

Statistical Analyses: The SAP was finalized prior to database lock and it included a more technical and detailed description of the statistical analyses described in this example. This example provides a summary of the statistical analyses of the most important endpoints including primary, secondary, and exploratory endpoints.

General Considerations: Descriptive statistics were used to summarize the results of the study. Continuous variables were summarized by reporting the number of observations, mean, standard deviation, median, minimum, and maximum. For the PK parameters and plasma concentrations, geometric mean and coefficient of variation were also presented. Categorical/discrete variables were summarized using frequency tables showing the number and percentage of patients within a particular category. All summaries were presented by dose group and treatment group.

Baseline values were taken as the most recent assessment prior to dosing, reported during screening and up to and including Day 1.

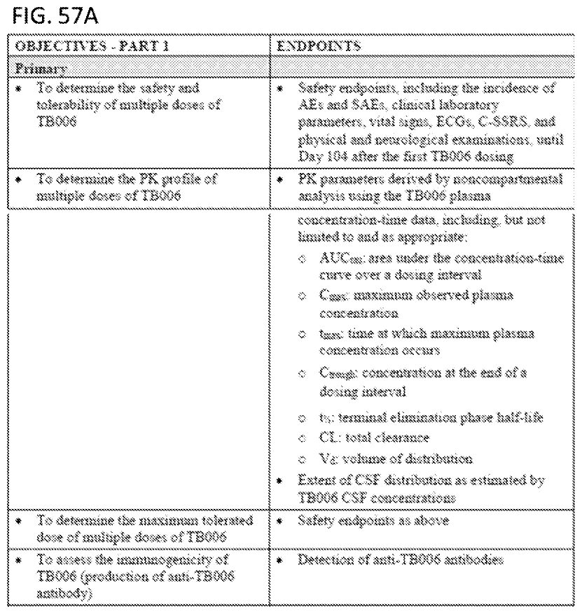

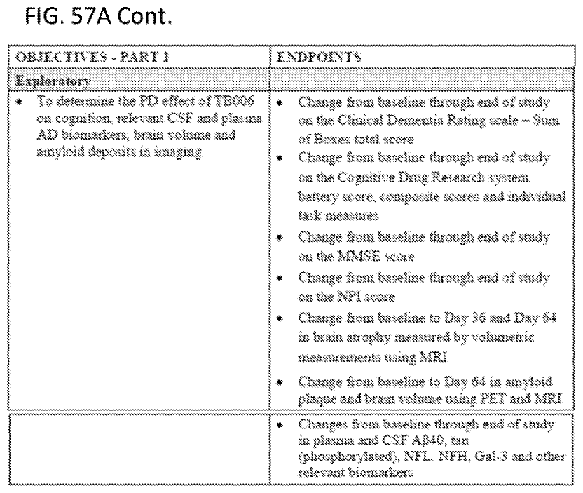

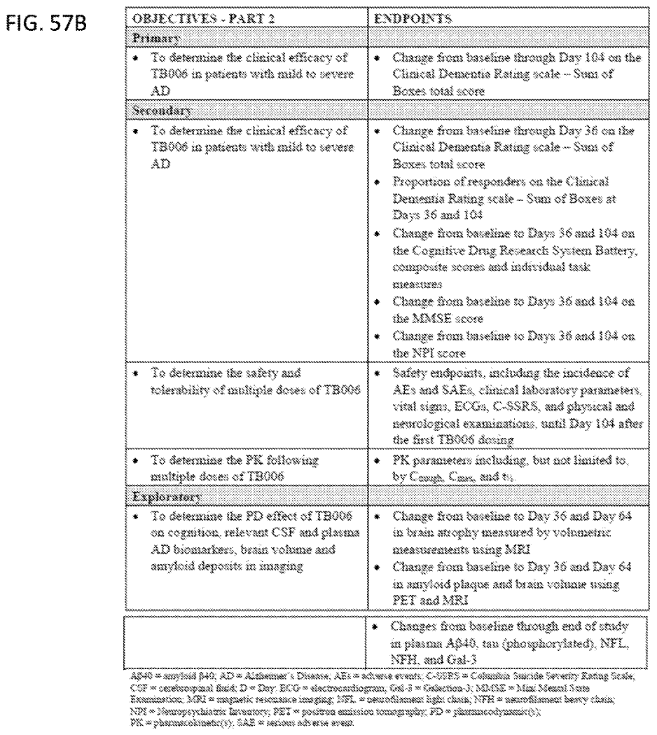

FIG. 57 is a table depicting some embodiments of endpoints in a study of the safety efficacy of anti-Gal3 antibodies. FIG. 57A is a table depicting some embodiments of endpoints in part one of a two part study of the safety efficacy of anti-Gal3 antibodies. FIG. 57B is a table depicting some embodiments of endpoints in part two of a two part study of the safety efficacy of anti-Gal3 antibodies.

### Part 1 Primary Endpoints:

Safety and Tolerability: All AEs were coded using the most recent version of MedDRA. The incidence of AEs was summarized by SOC and PT. Similar summaries were produced for AEs by severity, SAEs, treatment related AEs, and AEs leading to discontinuation. AEs were collected from the first dose of study drug, thus all AEs were considered treatment emergent AEs.

Table 8 depicts the frequency of AEs for study participants treated with TB006 or a placebo.

TABLE 8

| | TB006 (n = 80) | | Placebo (n = 73) |
|---|---|---|---|
| Patients with any AE, n (%) | 28 | (44%) | 17 (26%) |
| Patients with treatment- | 4^{a} | (6.3%) | 0 (0%) |
| related AE, n (%) | | | |
| Patients with an SAE, n (%) | 5 | (8%) | 1 (1%) |
| Patients with treatment- | 0 | (0%) | 0 (0%) |
| related SAE, n (%) | | | |
| Patients withdrawn from | 1^{b} | (1%) | 0 (0%) |
| study due to AE, n (%) | | | |

AE: Adverse event
SAE: Serious adverse event
4^{a} These four grade 1 AEs include one low energy, one phlebitis and two infusion reactions
1^{b} One patient withdrawn due to infusion reaction

Twenty eight patients (44%) in the active treatment group reported 52 AEs; whereas 17 patients, (26%) in the placebo group reported 30 AEs. The most common AEs reported in the “all” adverse events category were Covid-19, urinary tract infection, back pain, blood creatinine increase, infusion-related reaction, and upper respiratory infection in descending order and were the only AEs reported more than once. There were 4 “related” AEs (AEs thought to be either definitely or probably related to study drug treatment): infusion-related reaction (2), dizziness (1), and phlebitis (1). Five patients experienced serious adverse events (SAE) in the active treatment groups; 1 in the placebo group. None of these SAEs were considered “related” to study drug treatment by the investigator. There was one patient death and one SAE that led to study discontinuation, both in the active treatment group. There were no image-related abnormalities, either edema or hemorrhage.

Table 9 depicts the most frequent AEs for study participants treated with TB006 or a placebo.

TABLE 9
Most Frequent “All” Adverse Events in Descending Order

| | | |
|---|---|---|
| | TB006 1000 mg | Placebo |
| Preferred Term | (n = 63) | (n = 66) |

Most Frequent “All” Adverse Events in Descending Order

| | | |
|---|---|---|
| Covid-19 | 5 | 2 |
| Urinary tract infection | 3 | 1 |
| Back pain | 2 | 0 |
| Blood creatinine increased | 2 | 1 |
| Infusion-related reaction | 2 | 0 |
| Upper respiratory reaction | 2 | 0 |

Most Frequent “Related” Adverse Events in Descending Order

| | | |
|---|---|---|
| Infusion-related reaction | 2 | 0 |
| Dizziness | 1 | 0 |
| Phlebitis | 1 | 0 |

As is clear from Table 8 and Table 9, TB006 was well tolerated during the study.

The ECG results were presented by visit, summarizing the continuous measurements based on the average of triplicate values, including the QT, QTc, QRS, RR, PR intervals, as well as change from baseline values, and clinically significant changes in heart rate and rhythm.

Reported values and change from baseline values of hematology, clinical chemistry and urinalysis (and the determinations relevant to the normal ranges and appropriate clinically significant or CTCAE toxicity gradings) were summarized by laboratory test for each assessment day, using appropriate descriptive statistics.

Vital signs (systolic and diastolic blood pressure, pulse rate, respiratory rate, and oral temperature) were summarized by assessment day showing absolute values, change from baseline values, and clinically significant changes (including orthostasis) using appropriate descriptive statistics.

It is expected that treatment of subject's with anti-Gal3 antibody in part 1 and/or part 2 of the present example resulted in the subjects having improve, i.e., increased or decreased, MMSE, CDR-SB, EQ-5D-5L, NPI, Cognitive Drug Research battery, strength, balance, locomotor function, whole brain volume, brain atrophy, or any combination thereof.

### Example 14: Anti-Gal3 Antibodies Promote Increased Whole Brain Volume

The effect of TB006 on whole brain volume was assessed in subjects undergoing an open-label long term extension of the double-blind (TB006AD2102) study in patients with AD according to the protocol of Example 1, above.

Subject's whole brain volume were assessed by MRI. The MRI was be administered at the time points indicated in the SoA (FIG. 29). The procedure was be performed at a center associated with site 117. Site staff coordinated the scheduling of the MRI and may have been asked to accompany the patient and caregiver to the facility. The MRI was scheduled after other screening procedures had been completed to ensure a high likelihood of patient eligibility.

During the MRI, subjects were instructed to lie still in an enclosed environment for the duration, which was usually approximately 20 minutes. The subjects were informed that they may hear a buzzing sound throughout the procedure and may hear an occasional thumping sound.

Otherwise, the procedure was noninvasive and painless. After the procedure, the subjects may have needed a few minutes to reorient themselves in a sitting position.

Table 10 depicts the whole brain volume of 7 subjects at 3 different time points following anti-Gal3 antibody administration.

TABLE 10

| | MRI whole brain vol | | |
|---|---|---|---|
| Subject | D −1 | D 36 | D 64 |
| 1 | 10509.92 | 10555.83 | 10374.89 |
| 2 | 10326.19 | 10344.1 | 10297.75 |
| 3 | 10899.12 | 10980.56 | 10971.21 |
| 4 | 10279.44 | 10479.03 | 10646.04 |
| 5 | 8093 | 8044.32 | 8175.66 |
| 6 | 11772.73 | 11747.22 | 11895.73 |
| 7 | 9776.74 | 9893.73 | 9832.33 |
| 8 | 10509.64 | 10531.78 | 10409.11 |
| 9 | 9774.03 | 10149.21 | 10442.13 |
| 10 | 11177.52 | 11336.97 | 11183.86 |
| 11 | 11599.53 | 11629.12 | 11782.41 |

As is clear from Table 10, anti-Gal3 antibodies promote increased whole brain volume within at least 36 and 64 days of anti-Gal3 antibody administration.

### Example 15: Anti-Gal3 Antibodies Promote Decreased Clinical Dementia Rating Scale-Sum of Boxes (CDR-SB) Scores

The longitudinal effect of TB006 on cognition was assessed in subjects undergoing a phase 1b/2a, multi-dose, multi-center study to assess the safety, tolerability, pharmacokinetics, pharmacodynamics, and efficacy of TB006 in patients with Alzheimer's Discase.

FIG. 78 depicts some exemplary embodiments of TB006 light chain (SEQ ID NO.: 495) and heavy chain (SEQ ID NO.: 449) amino acid sequences.

The TB006 antibody was formulated as a clear to slightly opalescent, sterile solution for injection. The TB006 antibody is administered at a unit dose strength of 20 mg/ml (in some embodiments, the 20 mg/ml can be diluted to 8 mg/ml in a total of 500 mL prior to I. V. administration) in 8 mL vial (160 mg total). Prior to administration, the correct dosage of TB006 was added to 300 mL of sterile normal saline for injection, USP, for a total of 500 mL, and then infused over 1 hour with the use of a 0.2-micron filter, q28 day±5 days.

Patients performed a battery of cognition and quality of life tests, and biomarker testing, including the Clinical Dementia Rating Scale-Sum of Boxes (CDR-SB), the Mini Mental State Examination (MMSE, Cognitive Drug Research system (CDR) battery, and the Neuropsychiatry Inventory (NPI), and the EuroQuality of life (EuroQol) 5-Dimension 5-Level (EQ-5D-5L).

The Clinical Dementia Rating Scale is derived from a semi-structured interview with the participant and an appropriate informant, and it rates impairment in 6 categories (memory, orientation, judgment and problem solving, community affairs, home and hobbies, and personal care) on a 5-point scale for which 0=no impairment, 0.5=questionable impairment, and 1, 2, and 3=mild, moderate, and severe impairment, respectively.

Cognition and biomarker testing was performed in the SoA (FIG. 60). As can be seen in FIG. 60, a CDR-SB was administered at Day-1 to establish a base line, and then again at D15, Day 36, Day 64, and Day 104. The CDR-SB change from baseline through Day 104 was analyzed using Mixed Model Repeated Measures (MMRM) with treatment group, randomization strata (MMSE≤20 and 21-24), visit, and treatment group by visit interaction as fixed effects and baseline CDR-SB score as a covariate. This yields a least square mean difference, a 95% confidence interval of the difference, and a p-value of the difference.

A responder was defined as a 1-point sustained improvement from baseline. Patients with missing data were included in the denominator and treated as non-responders. All secondary endpoints except responder analyses were analyzed similarly using the MMRM with treatment group, randomization strata (MMSE≤20 and 21-24), visit, and treatment group by visit interaction as fixed effects and a corresponding baseline score as covariates. The proportion of the responders between treatment groups were tested using the Cochran Mantel-Haenszel test, adjusting for randomization strata (MMSE≤20 and 21-24), yielding a common risk difference, a 95% confidence interval, and a p-value of the difference.

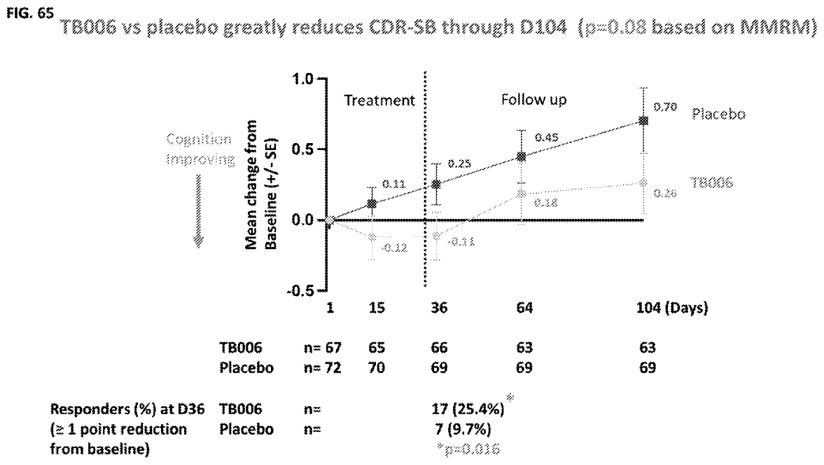

FIG. 65 is a line graph depicting the effect of TB006 on the global CDR-SB scores in patients undergoing a phase 1b/2a, multi-dose, multi-center study to assess the safety, tolerability, pharmacokinetics, pharmacodynamics, and efficacy of TB006 in patients with Alzheimer's Disease.

The CDR-SB has been proposed for use in longitudinal assessment of dementia and is widely used in AD studies as a global measure of disease progression (Williams et al, 2013).

The least squares (LS) mean on the change from baseline through Day 104 (primary endpoint) was +0.01 (±0.147), or 2.13% (LS mean and percentage change), for TB006 and +0.35 (±0.142), or 8.75%, for placebo, or a difference of-0.35 favoring TB006. The MMRM analysis test resulted in p=0.0838, approaching statistical significance. The nominal point estimate through Day 104 visit was +0.26 for TB006 and +0.7 for placebo, an even greater difference (−0.44 points) than the overall LS mean difference. The analysis at Day 36 showed a mean change of −0.16 (±0.162), indicating improvement, vs. +0.23 (±0.157) for placebo; a difference of 0.39 favoring TB006 (p=0.0773). This pattern favoring numerical superiority of TB006 vs placebo was fairly consistent across most of the CDR subdomains, including the global score.

On Day 104, there were 15 patients, (22.4%) meeting predefined criteria for CDR-SB response (±≥1.0 pt) in the TB006 group, vs. 11 patients (15.3%) in the placebo group. The difference between treatment groups did not reach statistical significance. However, on Day 36, the responders were 17 (25.4%) in the TB006 group and 7 (9.7%) in the placebo group, which was significant (p=0.0164).

In general, numerical differences between active and placebo across strata tended to be similar to slightly greater for the less severe population. For example, the differences at the Day 104 visit were 0.55 for MMSE 21-24 and −0.31 for MMSE<21. Similar observations are noted on the responder analyses.

As is clear from FIG. 65, TB006 significantly (p=0.01 based on MMRM) reduces the global CDR-SB score in patients as compared to the placebo, indicating that TB006 promotes the reversal and/or decreased progression of AD.

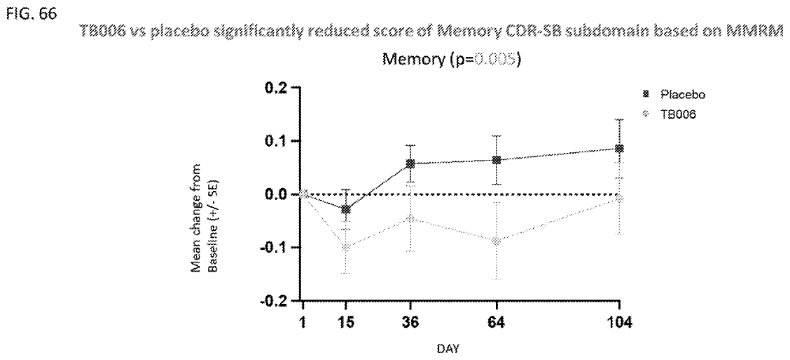

FIG. 66 is a line graph depicting the effect of TB006 on Memory CDR-SB subdomain scores in patients undergoing a phase 1b/2a, multi-dose, multi-center study to assess the safety, tolerability, pharmacokinetics, pharmacodynamics, and efficacy of TB006 in patients with Alzheimer's Disease.

As is clear from FIG. 66, TB006 significantly (p=0.005 based on MMRM) reduces the Memory CDR-SB subdomain score in patients as compared to the placebo, indicating that TB006 promotes increased memory, for example, reduced forgetfulness, in subjects with AD.

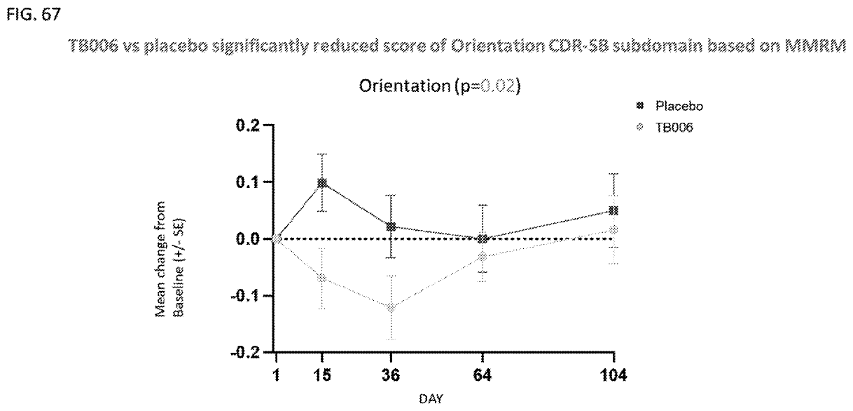

FIG. 67 is a line graph depicting the effect of TB006 on the Orientation CDR-SB subdomain scores in patients undergoing a phase 1b/2a, multi-dose, multi-center study to assess the safety, tolerability, pharmacokinetics, pharmacodynamics, and efficacy of TB006 in patients with Alzheimer's Disease.

As is clear from FIG. 67, TB006 significantly (p=0.02 based on MMRM) reduces the Orientation CDR-SB subdomain score in patients as compared to the placebo, indicating that TB006 promotes increased orientation, for example, an increased understanding of temporal and/or geographic relationships, in subjects with AD.

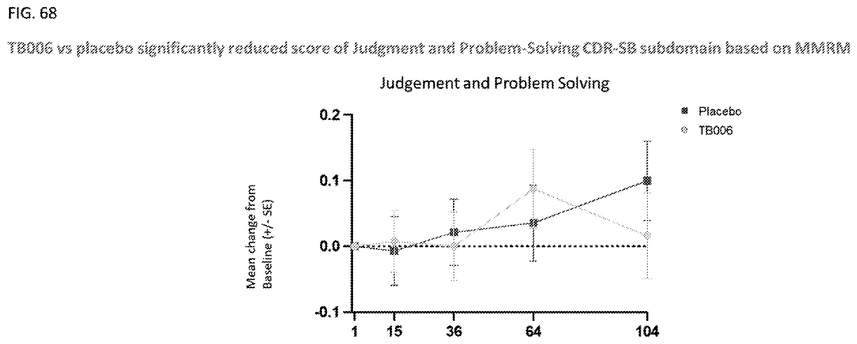

FIG. 68 is a line graph depicting the effect of TB006 on the Judgment and Problem Solving CDR-SB subdomain scores in patients undergoing a phase 1b/2a, multi-dose, multi-center study to assess the safety, tolerability, pharmacokinetics, pharmacodynamics, and efficacy of TB006 in patients with Alzheimer's Disease.

As is clear from FIG. 68, TB006 significantly reduces the Judgement and Problem Solving CDR-SB subdomain score in patients as compared to the placebo, indicating that TB006 promotes increased judgement and problem solving, for example, the ability to recognize similarities or differences, or exercise social judgement, in subjects with AD.

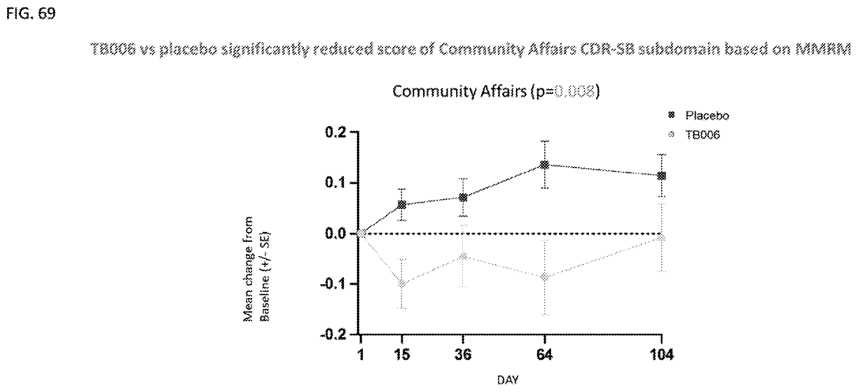

FIG. 69 is a line graph depicting the effect of TB006 on the Community Affairs CDR-SB subdomain scores in patients undergoing a phase 1b/2a, multi-dose, multi-center study to assess the safety, tolerability, pharmacokinetics, pharmacodynamics, and efficacy of TB006 in patients with Alzheimer's Disease.

As is clear from FIG. 69, TB006 significantly (p=0.008 based on MMRM) reduces the Community Affairs CDR-SB subdomain score in patients as compared to the placebo, indicating that TB006 promotes increased Community Affairs, for example, the ability to function independently at a job or shopping, in subjects with AD.

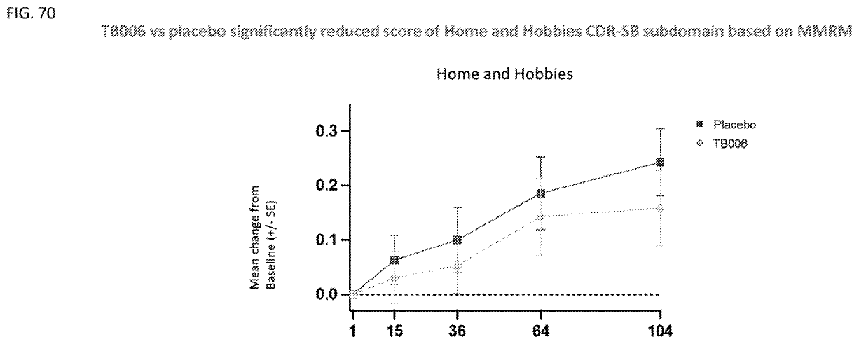

FIG. 70 is a line graph depicting the effect of TB006 on the Home and Hobbies CDR-SB subdomain scores in patients undergoing a phase 1b/2a, multi-dose, multi-center study to assess the safety, tolerability, pharmacokinetics, pharmacodynamics, and efficacy of TB006 in patients with Alzheimer's Disease.

As is clear from FIG. 70, TB006 significantly reduces the Home and Hobbies CDR-SB subdomain score in patients as compared to the placebo, indicating that TB006 promotes increased quality of home life and hobbies, for example, the ability to function independently at home, or maintain intellectual interests, in subjects with AD.

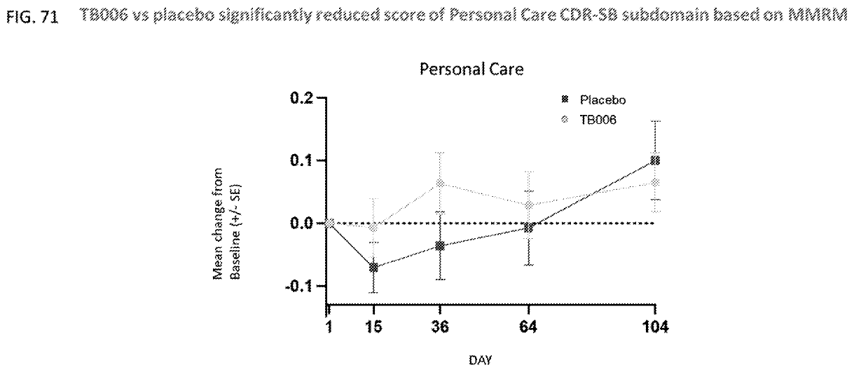

FIG. 71 is a line graph depicting the effect of TB006 on the Personal Care CDR-SB subdomain scores in patients undergoing a phase 1b/2a, multi-dose, multi-center study to assess the safety, tolerability, pharmacokinetics, pharmacodynamics, and efficacy of TB006 in patients with Alzheimer's Disease.

As is clear from FIG. 71, TB006 significantly reduces the Personal Care CDR-SB subdomain score in patients as compared to the placebo, indicating that TB006 promotes increased self-care in subjects with AD.

### Example 16: Anti-Gal3 Antibodies Promote Increased Mini-Mental State Examination Scores

The longitudinal effect of TB006 on cognition was assessed in subjects undergoing a phase 1b/2a, multi-dose, multi-center study to assess the safety, tolerability, pharmacokinetics, pharmacodynamics, and efficacy of TB006 in patients with Alzheimer's Disease according to the protocol of Example 13, above.

Patients performed a battery of cognition and quality of life tests, and biomarker testing, including the Clinical Dementia Rating Scale-Sum of Boxes (CDR-SB), the Mini Mental State Examination (MMSE), Cognitive Drug Research system (CDR) battery, and the Neuropsychiatry Inventory (NPI), and the EuroQuality of life (EuroQol) 5-Dimension 5-Level (EQ-5D-5L).

Cognition and biomarker testing was performed in the SoA (FIG. 60). As can be seen in FIG. 60, a MMSE was administered between Day-28 and again at Day-1 to establish a base line, and then again at D15, D36, D64, and D104.

FIG. 18D depicts a Mini-Mental State Examination (MMSE) score keeping template.

FIG. 72 is a line graph depicting the effect of TB006 on CDR-SB scores in patients undergoing a phase 1b/2a, multi-dose, multi-center study to assess the safety, tolerability, pharmacokinetics, pharmacodynamics, and efficacy of TB006 in patients with Alzheimer's.

The Mini-Mental State Examination (MMSE) is the best-known and the most often used short screening tool for providing an overall measure of cognitive impairment in clinical, research and community settings. Disease and is often used to evaluate the progression of AD.

The mean on the change from baseline at Day 104 was +0.89 (±0.377) for TB006 and +0.47 (±0.362), or a difference of +0.42 favoring TB006. The MMRM analysis result did not reach statistical significance. The point estimate at the Day 104 visit was +1.0 for TB006 and +0.6 for placebo, a difference (−0.40 points) favoring TB006. The analysis at Day 36 showed a mean change of +1.16 (±0.316) for TB006 vs. +0.14 (±0.306), a difference of 1.02 points favoring TB006. This difference was significant (p=0.02). The pattern of TB006 numerical superiority was consistent throughout nearly all the timepoints.

Numerical differences between strata tended to be similar. On Day 36, the numerical difference between active and placebo was slightly greater for the less severe population, while it was greater for the more severe group at the Day 104 visit.

As is clear from FIG. 72, TB006 significantly (p=0.01 based on MMRM) increases the MMSE score in patients treated with TB006 as compared to the placebo, indicating a reversal and/or decrease in the severity of AD progression in the subject.

### Example 17: Assessment of Plasma Biomarkers in Subjects Treated with an Anti-Gal3 Antibody

The effect of an anti-Gal3 antibody on plasma biomarkers Aβ40, tau (phosphorylated), neurofilament light chain (NFL), neurofilament heavy chain (NFH), and Galectin-3 (Gal-3), was evaluated in AD patients undergoing a phase 1b/2a, multi-dose, multi-center study to assess the safety, tolerability, pharmacokinetics, pharmacodynamics, and efficacy of TB006 in patients with Alzheimer's Disease according to the protocol of Example 13, above.

Venous blood samples of approximately 6 mL are collected for measurement of plasma Aβ40, tau (phosphorylated), NFL, NFH, Gal-3, and other relevant biomarkers at the timepoints specified in the SoA (FIG. 60). As can be seen in FIG. 60, blood samples were drawn at Day-1, Day 36, and Day 34.

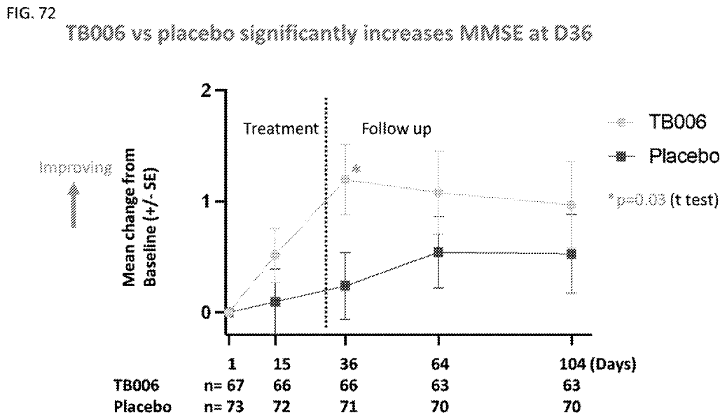

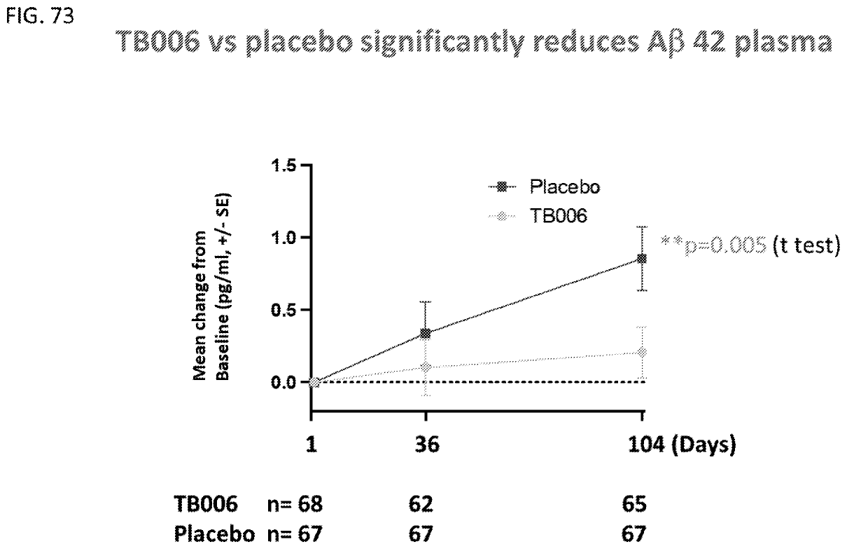

FIG. 73 is a line graph depicting the effect of TB006 on Aβ plasma levels in patients undergoing a phase 1b/2a, multi-dose, multi-center study to assess the safety, tolerability, pharmacokinetics, pharmacodynamics, and efficacy of TB006 in patients with Alzheimer's Disease.

As is clear from FIG. 73, TB006 significantly Aβ42 plasma levels in patients treated with TB006 as compared to the placebo.

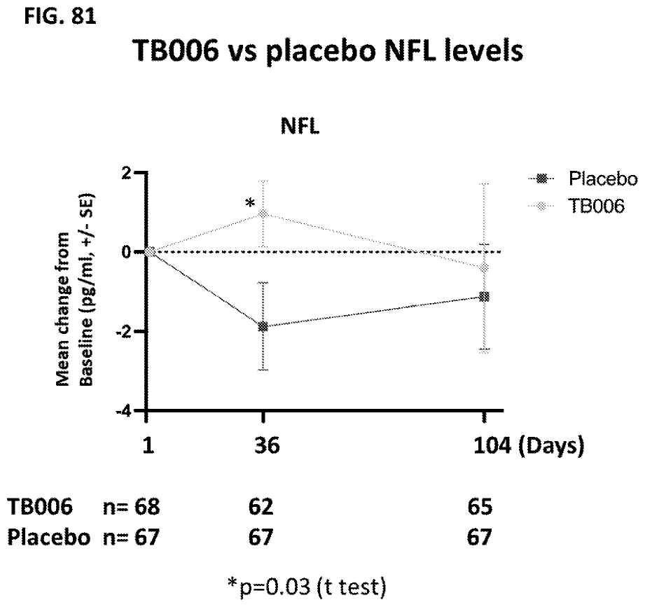

FIG. 81 is a line graph depicting NFL levels in patients treated with TB006 or placebo.

As is clear from FIG. 81, TB006 significantly increases NFL levels in AD patients by day 36. This appears to indicate that it is working, as it is expected that TB006 enhances clearance of NFL from the brain resulting in increased NFL levels initially following treatment with TB006. As can be seen in FIG. 81, NFL levels in patients treated with TB006 appear to decrease from day 36 to day 104 following treatment. It is expected that with longer term administration of TB006 to patients with AD will result in decreased NFL levels and further increased cognitive ability.

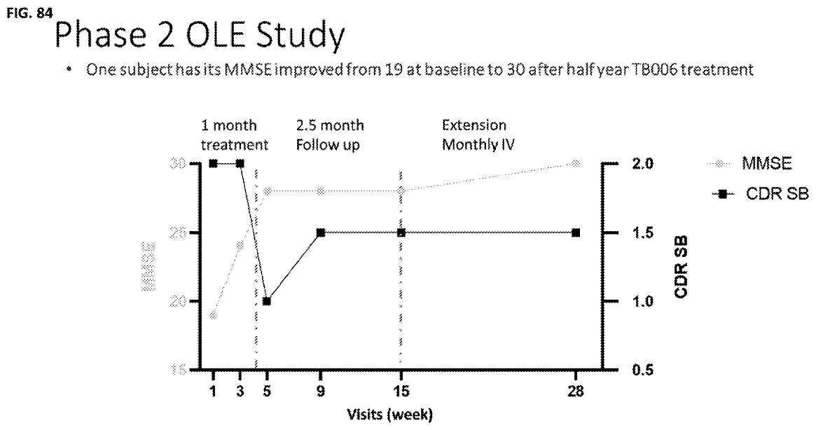

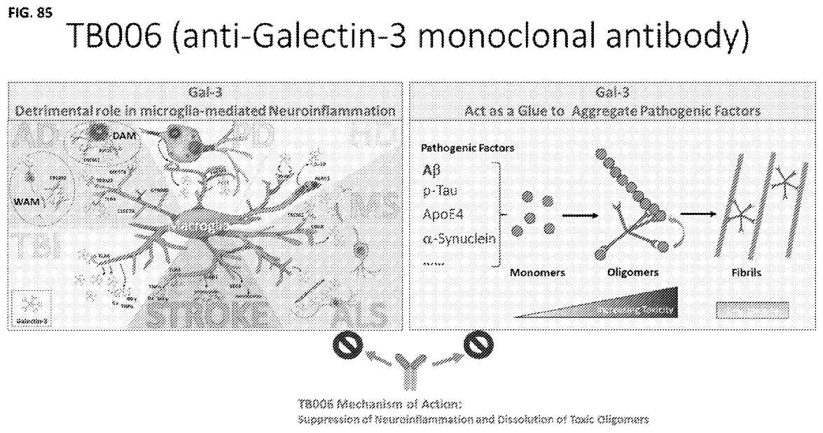

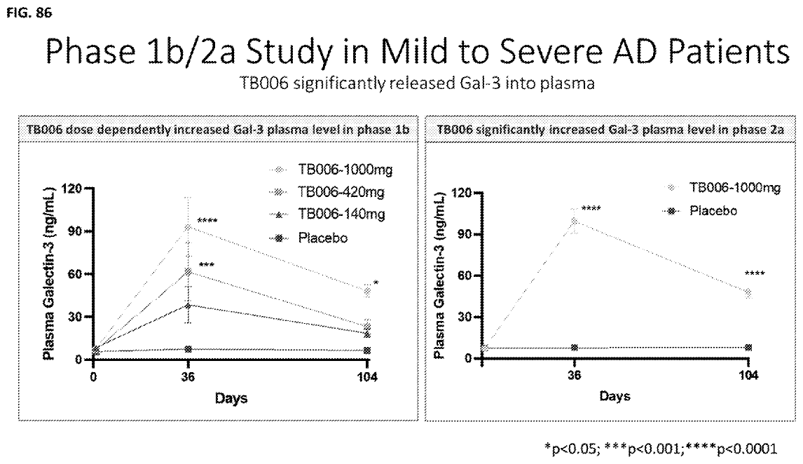

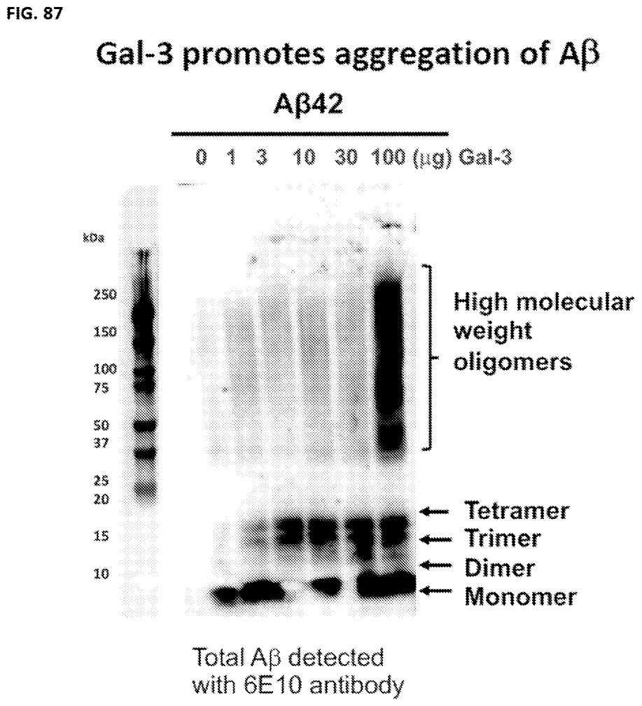

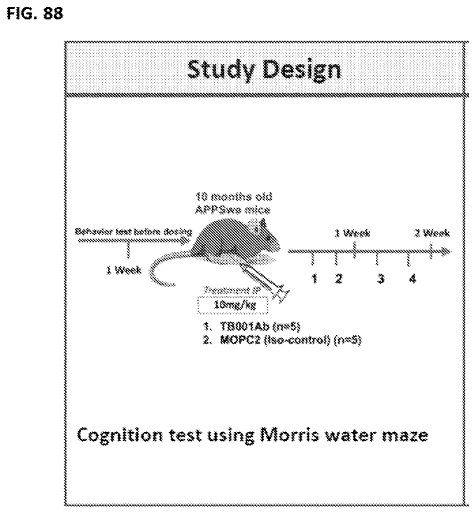

FIG. 86 is a pair of line graphs depicting a dose dependent increase in Gal-3 plasma levels in subjects with mild to severe AD during phase 1b (left) and phase 2a (right) treated with anti-Gal3 antibody.

As is clear from FIG. 86, subjects with mild to severe AD undergoing phase 1b and phase 2a experience a significant increase in plasma Gal3 levels by day 36. This appears to indicate that it is working, as it is expected that TB006 enhances clearance of Gal3 from the brain and or other organs resulting in increased plasma Gal3 levels initially following treatment with TB006. As can be seen in FIG. 86, Gal3 levels in patients treated with TB006 appear to decrease from day 36 to day 104 following treatment. It is expected that with longer term administration of TB006 to patients with AD will result in decreased Gal3 levels and further increased cognitive ability. As can be seen in the phase 1b subjects, the increase in plasma Gal3 levels is dose dependent suggesting that administration of higher doses of anti-Gal3 antibody may result in further clearance of Gal3.

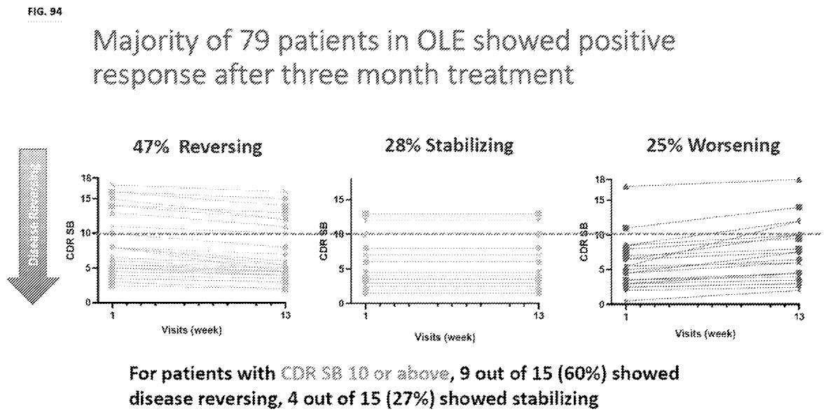

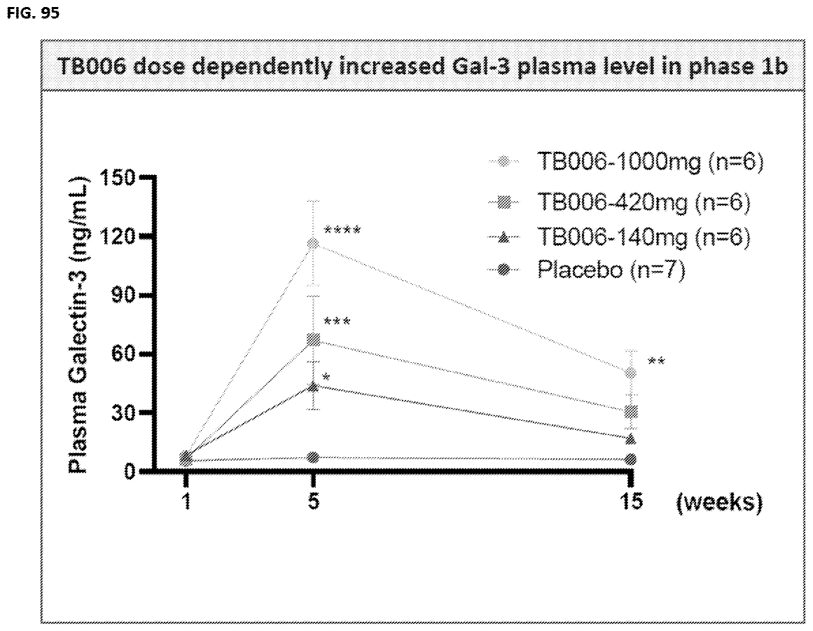

FIG. 95 is a line graph showing a dose dependent increase in plasma Gal3 levels in subjects treated with 0, 140, 420, or 1000 mg anti-Gal3 antibody.

TB006 treatment resulted in a dose-dependent increase in Gal-3 levels in Part 1 (Panel A), peaking at Day 36, where, not coincidentally, the highest TB006 concentrations of all sampling times occurred. The maximum mean concentrations at the highest dose level were ~120 ng/ml. All Gal-3 values at Day 36 and the 1000 mg dose at Day 104 were statistically greater than the placebo group, whose values remained at or near the baseline across all sampling times.

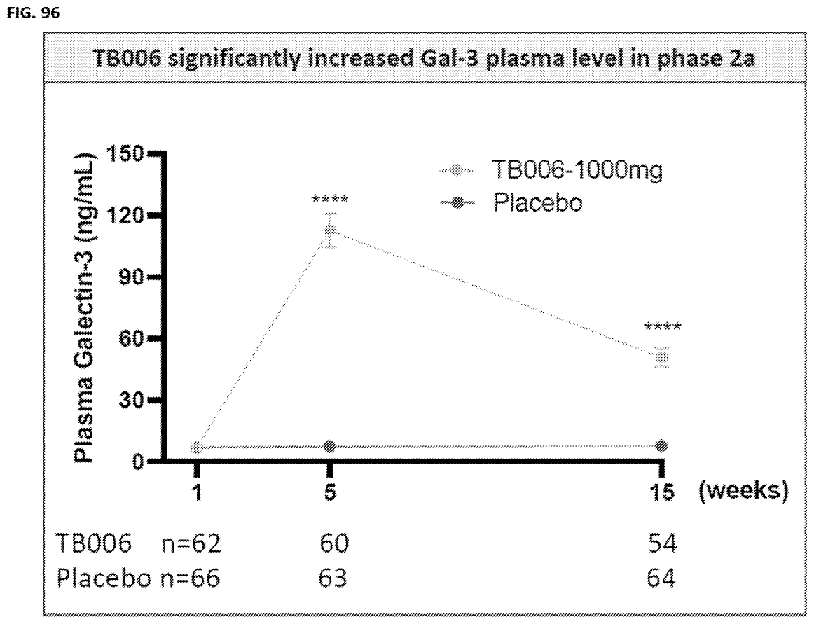

FIG. 96 is a line graph showing a significant increase in plasma Gal3 levels in subjects treated with 1000 mg anti-Gal3 antibody.

The profile of the mean plasma Gal-3 in Part 2, where the dose level of 1000 mg was used, was markedly similar to the 1000 mg dose level in Part 1.

As is clear from FIGS. 95 and 96, Gal3 plasma levels increase in a dose dependent manner to a significantly increased level over Gal 3 plasma levels in subject's treated with a placebo.

### Example 18: Assessment of Brain Atrophy and Amyloid Plaque Formation in Subjects Treated with an Anti-Gal3 Antibody

Brain atrophy is measured, by volumetric measurements, as well amyloid plaque formation, in AD patients undergoing a phase 1b/2a, multi-dose, multi-center study to assess the safety, tolerability, pharmacokinetics, pharmacodynamics, and efficacy of TB006 in patients with Alzheimer's Disease according to the protocol of Example 13, above.

Brain atrophy measured by volumetric measurements is assessed through magnetic resonance imaging (MRI). Amyloid plaque formation is assessed using positron emission tomography (PET).

Patients undergo imaging (PET and MRI) testing at months 3, 6, 12, and 24. These visits and all study assessments are presented in the SoA (FIG. 60).

The MRI is administered at the time points indicated in the SoA (FIG. 60). During the MRI, patients are instructed to lie still in an enclosed environment for the duration, which is usually approximately 20 minutes. Patients are informed that they may hear a buzzing sound throughout the procedure and may hear an occasional thumping sound. Otherwise, the procedure is noninvasive and painless. After the procedure, patients may need a few minutes to reorient themselves in a sitting position.

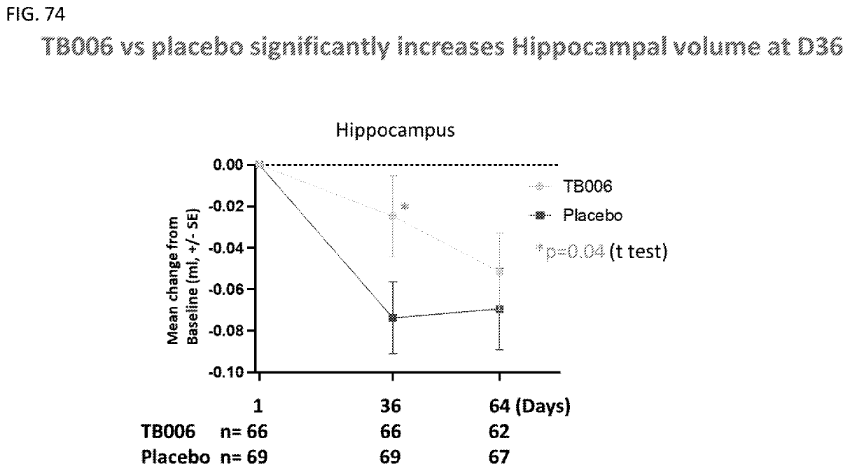

FIG. 74 is a line graph depicting the effect of TB006 on hippocampal volume in patients undergoing a phase 1b/2a, multi-dose, multi-center study to assess the safety, tolerability, pharmacokinetics, pharmacodynamics, and efficacy of TB006 in patients with Alzheimer's Disease.

As is clear from FIG. 74, TB006 significantly increases hippocampal volume as compared to placebo indicating that TB006 reduces brain atrophy and increases cognitive ability in patients with Alzheimer's disease.

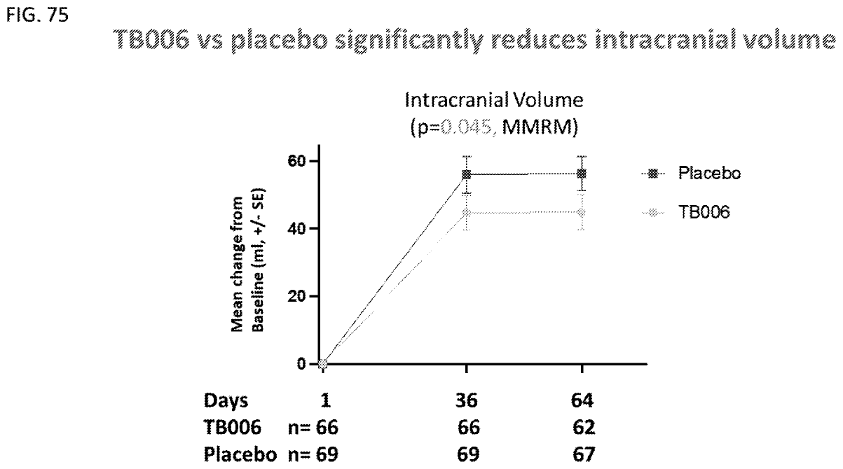

FIG. 75 is a line graph depicting the effect of TB006 on intracranial volume in patients undergoing a phase 1b/2a, multi-dose, multi-center study to assess the safety, tolerability, pharmacokinetics, pharmacodynamics, and efficacy of TB006 in patients with Alzheimer's Disease.

As is clear from FIG. 75, TB006 significantly decreases intracranial volume as compared to placebo indicating that TB006 reduces brain atrophy and increases cognitive ability in patients with Alzheimer's disease.

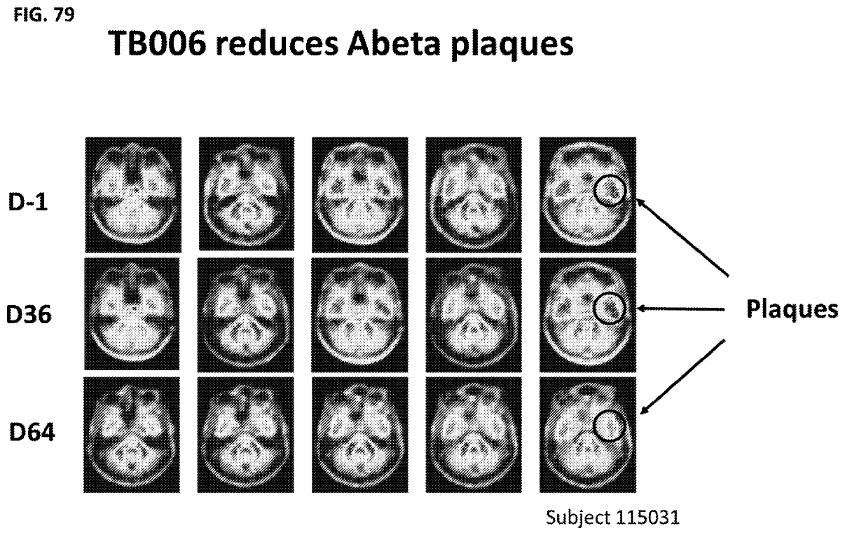

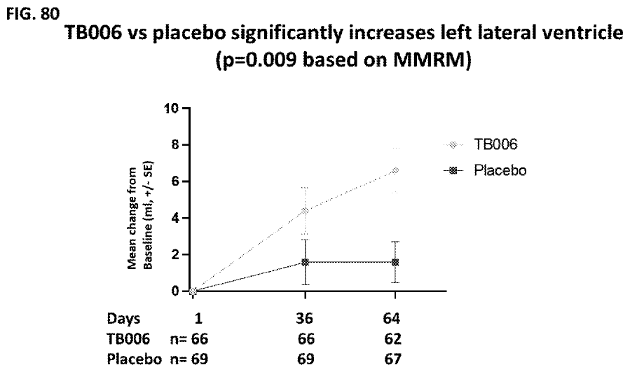

FIG. 80 is a line graph depicting the effect of TB006 on left lateral ventricle volume in patients undergoing a phase 1b/2a, multi-dose, multi-center study to assess the safety, tolerability, pharmacokinetics, pharmacodynamics, and efficacy of TB006 in patients with Alzheimer's Disease.

As is clear from FIG. 80, TB006 significantly increases left lateral ventricle brain volume as compared to placebo within days 0 to 64 following administration of TB006. TB006 was administered for only one month, with significant improvement on one or more cognitive tests by day 36. It is expected that long term administration will result in decreased left lateral ventricle volume further reducing brain atrophy and increasing cognitive ability in patients with Alzheimer's disease. Thus, TB006 works not only to treat the symptoms of AD but also to reverse the progression of AD.

Amyloid plaque formation is assessed using positron emission tomography (PET). The PET scan is performed at the same time points as the MRI, as indicated in the SoA (FIG. 60). Patients undergoing PET scans may be asked to fast for 6 hours prior to the procedure, depending on the requirements of the facility. They may also be asked to reduce carbohydrate intake for 24 hours prior to the procedure. A central reader may be used in this study to interpret MRI and PET changes in amyloid deposits, volume, and other important clinical endpoints.

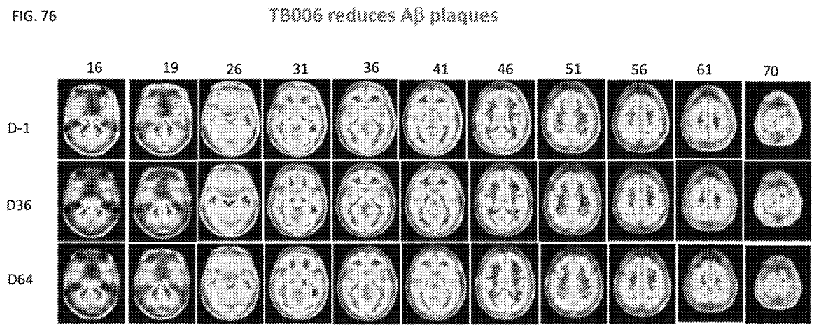

FIG. 76 depicts PET scans of subjects at different timepoints prior to and following treatment with TB006.

FIG. 79 PET scans of a subject at different timepoints following treatment with TB006.

Approximately 23% of the study population were characterized as amyloid positive, which, for purposes of this study, was defined as: 1.Two or more brain areas (each larger than a single cortical gyrus) in which there was reduced or absent gray white contrast, or 2. One or more areas in which gray matter radioactivity was intense and clearly exceeds radioactivity in adjacent white matter.

Overall, PET results showed no significant changes in amyloid content in the active treatment group vs. placebo in the span of 64 days following the first dose. Follow-up PET scans were performed on all patients regardless of baseline status. In patients who were amyloid positive at baseline, a trend of plaque reduction was observed in several subjects at D36 and D104.

As is clear from FIG. 76 and FIG. 77, TB006 reduces plaque formation in patients with AD.

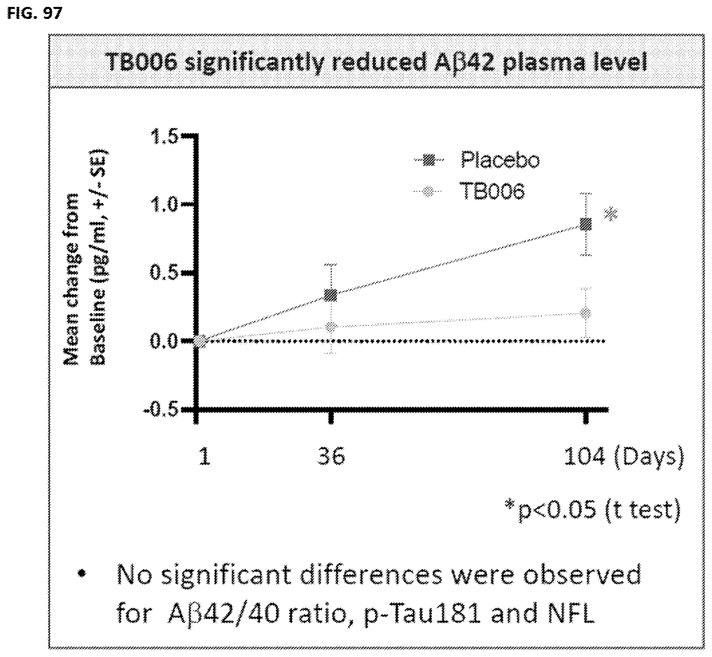

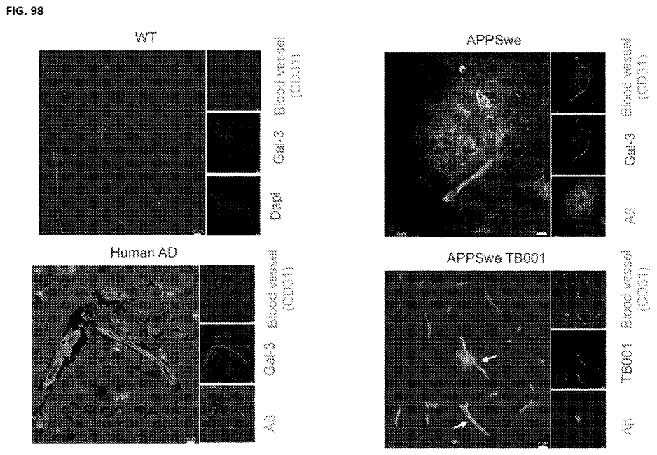

FIG. 97 is a line graph showing a reduction in Aβ plasma levels subjects treated with anti-Gal3 antibodies.

Patients in the placebo group demonstrated rising plasma Aβ42 throughout the follow-up period, while mean values in the TB006 1000 mg dose group remained virtually unchanged. The differences by Day 104 reached statistical significance.

### Example 19: A Phase 1 Double Blind, Randomized, Single Dose, Sequential Dose Escalation Study to Assess the Safety, Tolerability and Pharmacokinetics of TB006 in Healthy Subjects

This was a single center, randomized, double blind, placebo controlled, SAD escalation study design. The study (TB006HV1101) consisted of 5 dose levels of TB006 in healthy subjects and 1 ethno bridging group in healthy subjects of Chinese descent. Groups were enrolled sequentially after a review of the safety and tolerability of the previous group by a blinded Safety Review Committee. Dose levels were 70 mg, 200 mg, 700 mg (also used for the ethno-bridging group), 2100 mg, and 5000 mg. Eight subjects were enrolled into each dose group; 6 subjects in each group received active TB006 treatment, and 2 subjects received placebo.

Subjects remained at the clinical unit for 8 days following their dosing for safety assessments and PK/PD sampling. Subjects returned to the clinical unit on pre specified days for safety assessments and PK/PD sampling for 75 days. PD results are not reported at this time.

Forty-seven subjects were randomized in this study: 35 received TB006 and 12 received placebo. Subject demographics across Groups 1-5 were similar with respect to age, weight, gender, and BMI; with the exception of Group 1 which was almost entirely female (versus 55.3% male for the entire study).

Of the 35 subjects who received TB006, 17 (48.6%) (38 total AEs) versus 2 of 12 (16.7%) receiving placebo reported at least one AE. Of the 38 AEs in the TB006 group, 11 (31.4%) were Grade 1, 7 (20%) were Grade 2 and 7 (20%) were Grade 3. The AEs in the placebo group were Grade 1 and Grade 2 (1 each). Of the 38 AEs in the TB006 group, 17 (44.7%) were considered possibly, probably or definitely related to TB006, versus 21 (55.3%) that were unlikely or definitely not related. There does not appear to be a dose relationship associated with AE frequency. The most common AEs in the TB006 group (and the only AEs reported more than once) were headache (8), nausea (4) and back pain (2). The AEs in the placebo group were Covid-19 and rash (1 each). The AE profile in Group 6 appears similar to the other groups.

Although none were considered clinically significant, there were 10 potentially clinically significant lab abnormalities in the TB006 group, versus 1 in the placebo group. The abnormalities were increases in ALT and AST (2 each), increase in CPK (4), and increases in potassium and erythrocyte count (1 each). The abnormality in the placebo group was an increase in erythrocyte count. Three patients in the TB006 group versus none in the placebo group had marked liver abnormalities. Two of these were in Group 6.

Marked vital sign changes were similar between the TB006 and placebo groups, with a similar pattern in Group 6. Nine subjects (25.7%) experienced a QTcB increase of 30-60 msec from baseline in the TB006 group versus 1 (8.3%) in the placebo group. There were no other QTc abnormalities observed with the exception of 1 placebo subject who had a QTcB>450 msec. In general, across all dose groups and across all time points, subjects receiving TB006 experienced either a very slight increase or a reduction in QTcB compared to placebo subjects. This includes the 2 and 6 hr. time points, the time of maximum TB006 concentration.

The mean Cmax ranged from 25.35 μg/mL at the 70 mg dose to 1377.33 ug/mL at the 5000 mg dose. The Cmax was dose proportional (±10%) across all dose groups until the highest dose where the Cmax was slightly more than 15% less than dose proportional. The variability of the mean Cmax was low; % CV ranged from ~16%-28% across all dose groups. Mean Tmax ranged from 2.10 to 6.37 hr., with no dose-related pattern.

Exposure as measured by AUC (0-29) ranged from 7023.53 h*ug/mL at the 70 mg dose to 395387 h*ug/mL at the 5000 mg dose. Exposure was also dose proportional (±10%) across all dose groups until the 5000 mg dose, where they were slightly over 20% less than dose-proportional. This can be explained by a higher Vd at the 5000 mg dose (7.41 L) compared to a range of 4.32-5.67 across the other dose groups. Clearance was consistent across doses, ranging from 4.24 to 6.07 mL/hr. Variability was also very low with AUC; % CV ranged from 9.04% to 27.02%. This pattern of dose proportionality and low variability suggests a well-behaved PK profile.

Elimination half-life increases with each dose level, starting at a low of 28.4 days at the 70 mg dose to a high of 55.8 days at the 5000 mg dose. TB006 is suitable for once monthly administration.

In general, exposures in Group 6 were approximately 25% greater than the same dose in the general population and dose proportionality across all doses. Clearance (5.07 ml/hr.) was within the range of the general population, however Vd (3.92 L) was lower. The half-life (22.4 days) was also shorter than that of the general population.

There were 6 treatment groups: the first five were healthy volunteers from the general population; the last group were healthy volunteers of Chinese descent.

Single doses of TB006 as high as 5000 mg were safe and well tolerated through the observation period of 75 days. The maximum tolerated dose was not determined. Adverse events were generally mild, sporadic, and self-limiting. Headache was the most common adverse event reported. There were no adverse safety findings associated with clinical labs, vital signs, or ECGs.

The pharmacokinetic profile of TB006 followed a pattern of dose-proportionality, low variability, and predictability. Exposures in volunteers of Chinese descent were approximately 25% greater than the general population.

TB006 was safe and well tolerated in single doses up to 5000 mg. The observed safety profile suggests that TB006 is adequately safe for further use.

### Example 20: Anti-Gal3 Antibodies are Effective at Promoting Decreased CDR-SB Scores and Increased MMSE Scores in Patients with Alzheimer's Disease for Up to at Least 28 Weeks of Treatment

A Seamless Phase 1b/2a Double-blind, Randomized, Multiple dose, Multi-center, Sequential Dose-escalation Study (Example 13) was conducted to Assess the Safety, Tolerability, Pharmacokinetics, Pharmacodynamics and Efficacy of TB006 in Patients with Mild to Severe Alzheimer's Disease. Part 1 was a-MAD escalation study (Example 19) to evaluate the safety, tolerability, PK, PD, and efficacy of TB006 in patients with AD. Part 2 was a multi-center, randomized, double-blind, placebo-controlled, parallel-group study to evaluate the efficacy of TB006 after 1 month of treatment. Patients received a total of 5 doses, given in qw intervals. In Part 1, the starting dose in Group 1 was 140 mg qw for 5 weeks. Subsequent doses were 420 and 1,000 mg qw for Groups 2 and 3, respectively. The planned dose in Part 2 was the highest safe and well-tolerated dose from Part 1 (1,000 mg). All doses were infused IV over 1 hour.

Patients who have completed either Part 1 or Part 2 were then eligible to participate in an open-label long term extension (OLE) of the double-blind study in patients with AD according to the protocol of Example 1, above. Patients with different comorbidities like, diabetes, and hypertension were eligible and included. For some patients, enrollment into this study began immediately upon completion of the lead-in study. For those patients, the EoS procedures conducted in Example 13 was used as baseline values for this study.

During the OLE study, patients were administered TB006 according to FIG. 27. All patients received TB006 at a dose of 4000 mg via a 1-hour continuous IV infusion q28 day (±5 days). Patients were observed in the clinic for at least 2 hours following the end of the infusion for observation and safety assessments.

Patients returned to the clinic every 28 days for dose administration and safety assessments. All visits had a window of 11 days (±5 days from scheduled visit). This window was based on the original schedule at the start of the study, planned every 28 days, so if a patient deviated within the window, the original schedule was retained.

Approximately every 3 months, patients performed a battery of cognition, quality of life tests and biomarker testing, including the MMSE, the CDR-SB, the CDR, the EQ-5D-5L, and the NPI.

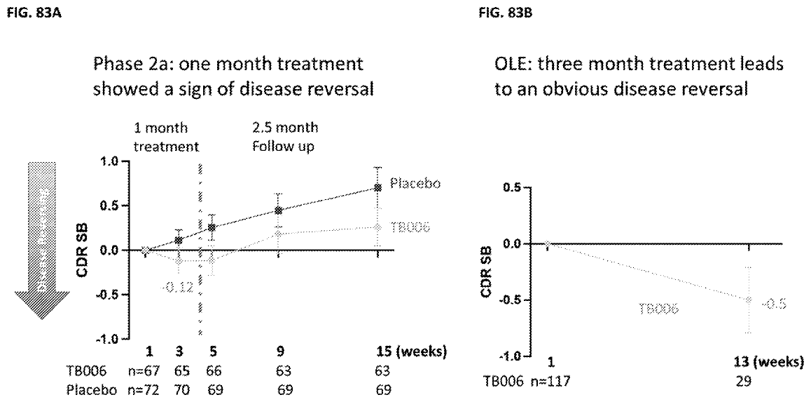

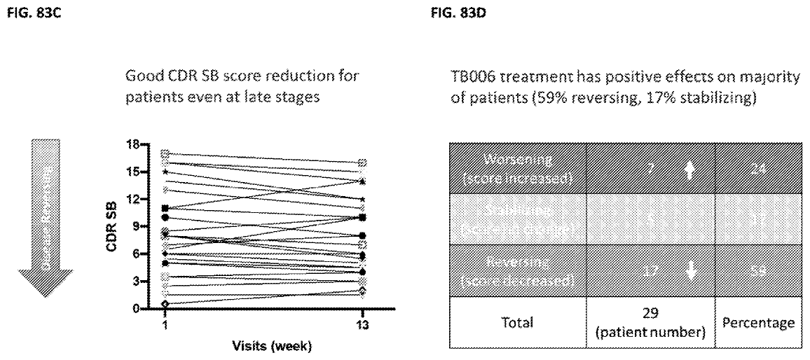

FIG. 83A is a line graph depicting the change in CDR-SB score of subject's with mild to severe AD treated with an anti-Gal3 antibody beginning at Part-Two of a seamless phase 1b/2a double-blind, randomized, multiple dose, multi-center, sequential dose-escalation study through the first battery of cognition, quality of life tests and biomarker testing at approximately three months into an open-label extension study.

As can be seen in FIG. 83A, subject's CDR-SB scores decrease by 0.12 points from baseline during 1 month of qw administration of 1000 mg of TB006. The subjects' CDR-SB scores remain decreased as compared to subject's receiving only placebo for the entirety of the 2.5 month follow up period. This indicates that TB006 is effective at decreasing CDR-SB scores in patients with mild to severe AD.

Following the 2.5 month follow up period, the subject entered the OLE portion of the study and received TB006 at a dose of 4000 mg via a 1-hour continuous IV infusion q28 day (±5 days). The subject was observed in the clinic for at least 2 hours following the end of the infusion for observation and safety assessments.

FIG. 83B is a line graph depicting the aggregate change in the baseline CDR-SB score of patients with mild to severe AD following the first battery of cognition, quality of life tests and biomarker testing at approximately three months into an open-label extension study.

As can be seen in FIG. 83B, the subjects' aggregate CDR-SB score decreased further following 1-month of TB006 administration at a dose of 4000 mg. This indicates that TB006 remains effective at decreasing CDR-SB scores even after multiple administrations. As can be seen in FIG. 83C, CDR SB reduction happened in mild, moderate and even in severe patients.

FIG. 83C is a line graph depicting the change in the global CDR-SB score of individual subjects with mild to severe AD following the first battery of cognition, quality of life tests and biomarker testing at approximately three months into the OLE study.

FIG. 94 is a trio of plots showing the change in CDR score in subjects having decreased (disease reversing), stabilized, or increased (disease worsening) CDR scores in some embodiments of subjects treated with an anti-Gal3 antibody.

FIG. 83D is a chart the total number and percentage of subjects individual subjects with mild to severe AD having improved, the same, or worsened cognition as indicated by the change in CDR-SB score from baseline to the first battery of cognition, quality of life tests and biomarker testing at approximately three months into the OLE study.

As can be seen in FIG. 83C, FIG. 94, and FIG. 83D, 59% of subjects with mild to severe AD have improved cognition following 3 once-monthly administrations of TB006. 17% of subjects with mild to severe AD have stabilized cognition following 3 once-monthly administrations of TB006. This indicates that TB006 remains effective following repeated administrations. It is expected that further improvements in cognition may be observed following the battery of cognition, quality of life tests and biomarker testing at later timepoints during the study.

FIG. 84 is a line graph depicting the change in MMSE score (left Y-axis) and Global CDR-SB score (right Y-axis) of an individual subject with mild to severe AD following beginning at Part-Two of the seamless phase 1b/2a double-blind, randomized, multiple dose, multi-center, sequential dose-escalation study through the first battery of cognition, quality of life tests and biomarker testing at approximately three months into an OLE study.

As can be seen in FIG. 84, the subject's MMSE score increases, i.e., improves, from 19 at the start of Part-Two to greater than 2.5 following 1 month of qw administration of 1000 mg of TB006. The subject's MMSE score remains elevated for the entirety of the 2.5 month follow up period. This indicates that TB006 is effective at improving MMSE scores in patients with mild to severe AD and that this improvement in MMSE score is stable. That is, the subject's MMSE score does not decrease following cessation of the TB006 antibody for at least 2.5 months.

Following the 2.5 month follow up period, the subject entered the OLE portion of the study and received TB006 at a dose of 4000 mg via a 1-hour continuous IV infusion q28 day (±5 days). The subject was observed in the clinic for at least 2 hours following the end of the infusion for observation and safety assessments. As can be seen in FIG. 84, the subject's MMSE score improved further to 30, a score of normal cognition, following 3-month of TB006 administration at a dose of 4000 mg. This indicates that TB006 becomes more effective at increasing MMSE score after multiple administrations.

TB006 is also effective at improving a subject's CDR-SB score. As can be seen in FIG. 84 the subject's CDR-SB score decreased from about 2.0 to about 1.0 following 1 month of qw administration of 1000 mg of TB006. The subject's CDR-SB score remains decreased throughout the entirety of the 2.5 month follow up period. This indicates that TB006 is effective at decreasing CDR-SB scores in patients with mild to severe AD and that this reduction in CDR-SB score is stable. That is, the subject's CDR-SB score does not return to baseline following cessation of the TB006 antibody for at least 2.5 months.

Following the 2.5 month follow up period, the subject entered the OLE portion of the study and received TB006 at a dose of 4000 mg via a 1-hour continuous IV infusion q28 day (±5 days). The subject was observed in the clinic for at least 2 hours following the end of the infusion for observation and safety assessments. As can be seen in FIG. 84, the subject's CDR-SB score was maintained at the decreased level following 3-months of TB006 administration at a dose of 4000 mg. This indicates that TB006 remains effective at decreasing and maintaining CDR-SB scores after at least 28 weeks of TB006 administration.

### Example 21: Anti-Gal3 Antibodies Reverse Cognition Loss in Mouse Models of Alzheimer's Disease

The effect of anti-Gal3 antibodies on cognition loss in mouse models of Alzheimer's disease was evaluated.

Ten month old APSSwe Alzheimer's disease model rats underwent cognition testing via a MWM approximately 1-week prior to anti-Gal3 antibody dosing. Cognition testing was performed via MWM. One-week after baseline cognition testing, 10 mg/kg TB001 anti-Gal3 antibody was administered to the mouse subject. The MWM was then administered each day for 6 days following administration of the anti-Gal3 antibody.

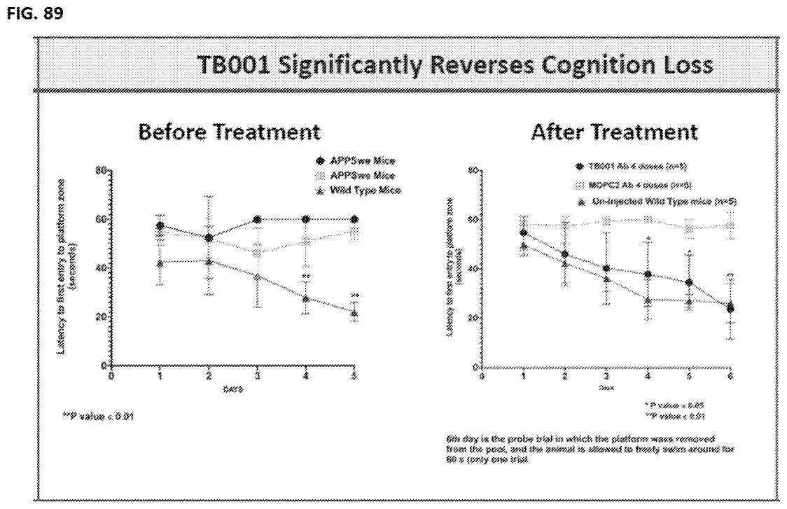

FIG. 89 is a pair of line graphs depicting reversal of cognition loss in an Alzheimer's disease mouse model before and after treatment with or without an anti-Gal3 antibody.

As is clear from FIG. 89, TB001 significantly reverses plaque formation and cognition loss in APPSwe AD mouse models treated with anti-Gal3 antibodies.

Following behavior testing, mice were sacrificed. Mice were anesthetized with isoflurane, and were perfused transcardially with cold PBS. Right hemisphere was processed for immunohistochemistry and left hemisphere was flash-frozen. Brain tissues were fixed for 48 hours with 4% paraformaldehyde (PFA) in PBS, pH 7.4 at 4° C. and stored in PBS/0.02% sodium azide (NaN3) at 4° C. until use.

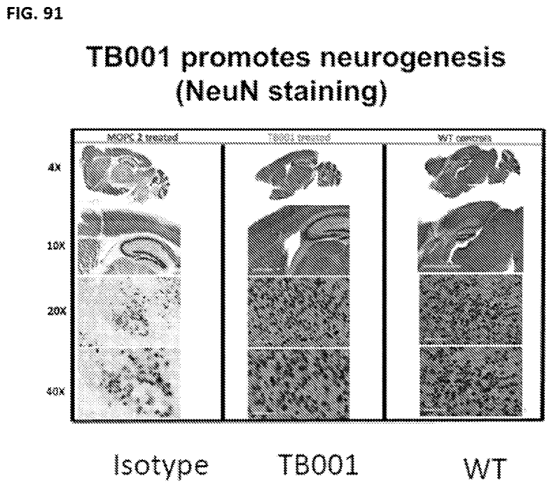

FIG. 91 shows representative images of anti-Gal3 antibody promotion of neurogenesis in NeuN stained brain tissue from an Alzheimer's disease mouse model before and after treatment with or without an anti-Gal3 antibody that has been stained for neuronal markers.

As is clear from FIG. 91, anti-Gal3 antibodies promote neurogenesis in AD mouse models.

Fixed brain tissues were sectioned (40 μm) with a vibratome. Sagittal sections were collected in PBS (containing 0.02% NaN3) and stored at 4° C. prior to staining. To stain for Aβ plaques, sections were immersed in 70% formic acid for 5 min. Endogenous peroxidase in tissue was blocked by treating with 3% H2O2 for 20 min at room temperature. Nonspecific background staining was blocked by 1 hour incubation in 2.5% serum in which the secondary antibody was raised, 0.2% Triton X-100 (TX) at room temperature. Tissues were incubated with primary Iba-1, neuronal NeuN (Abcam) antibodies in 2.5% serum overnight at 4° C., rinsed three times with PBS, 0.1% TX, followed by biotinylated secondary anti-mouse or anti-rabbit antibody detection with an ABC peroxidase kit (Vector Laboratories), and visualization with a 3,3′-diaminobenzidine (DAβ) substrate kit (Vector Laboratories). After DAβ staining, brain tissues were mounted on super frost plus microscopic slides (ThermoFisher Scientific) and dehydrated using different percentages of alcohols and xylenes. Slides were cover-slipped using DPX mounting media (Millipore Sigma).

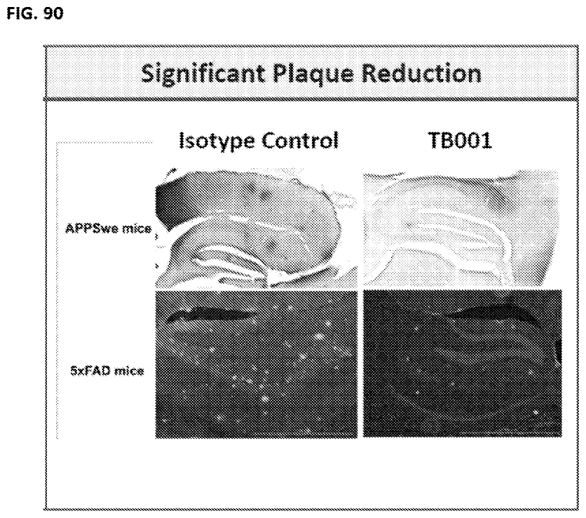

FIG. 90 shows representative images of plaque reduction in an Alzheimer's disease mouse model before and after treatment with or without an anti-Gal3 antibody.

As is clear from FIG. 90, Gal-2 promotes aggregation of Aβ in AD mouse models.

Control experiments with primary antibody omitted resulted in no staining.

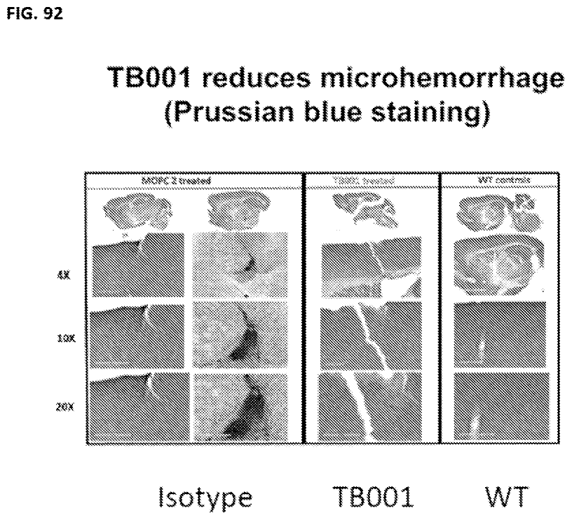

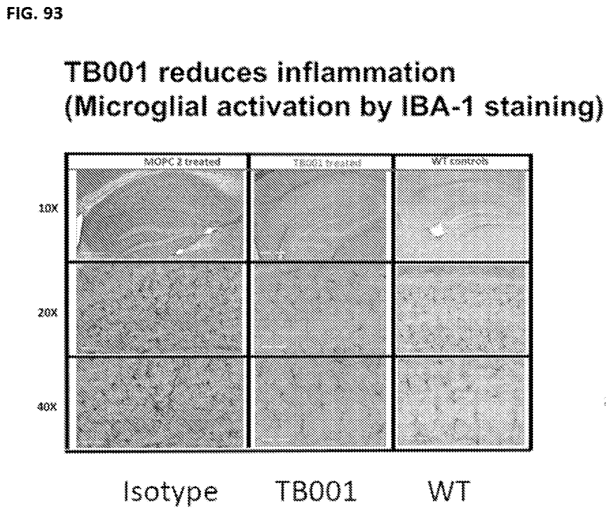

FIG. 92 shows representative images of anti-Gal3 antibody reduction of microhemorrhages in Prussian blue stained brain tissue in brain tissue from an Alzheimer's disease mouse model before and after treatment with or without an anti-Gal3 antibody.

Determination of hemorrhages was performed using Prussian blue staining of ferric acid. Brain sections were mounted on Super frost Plus microscopic slides. Staining was performed using Prussian blue solution, i.e., freshly made 5% potassium hexacyanoferrate trihydrate and 5% hydrochloric acid (Sigma). 30 min later, sections were rinsed in water, and counterstained with Nuclear Fast Red (Abcam), dehydrated and covered using DPX. Brain tissues were scanned using Aperio VERSA Brightfield, Fluorescence & FISH Digital Pathology Scanner (Leica Biosystems). NIH Image J software was used to quantify the histology. Quantification of percentage area was determined using color images converted to binary images and level threshold background subtracted.

As is clear from FIG. 92, TB001 reduces microhemorrhage in in AD mouse models.

### Example 22: Anti-Gal3 Antibodies Effect on NPI Scores

The longitudinal effect of TB006 on cognition was assessed in subjects undergoing a phase 1b/2a, multi-dose, multi-center study to assess the safety, tolerability, pharmacokinetics, pharmacodynamics, and efficacy of TB006 in patients with Alzheimer's Disease according to the protocol of Example 13, above.

Patients performed a battery of cognition and quality of life tests, and biomarker testing, including the Clinical Dementia Rating Scale-Sum of Boxes (CDR-SB), the Mini Mental State Examination (MMSE), Cognitive Drug Research system (CDR) battery, and the Neuropsychiatry Inventory (NPI), and the EuroQuality of life (EuroQol) 5-Dimension 5-Level (EQ-5D-5L).

Cognition and biomarker testing was performed in the SoA (FIG. 60). As can be seen in FIG. 60, a MMSE was administered between Day-28 and again at Day-1 to establish a base line, and then again at D15, D36, D64, and D104.

The Neuropsychiatric Inventory is a rater-administered, fully structured interview administered to a caregiver who knows the patient well. It has 10 behavioral domains: delusions, hallucinations, agitation/aggression, depression/dysphoria, anxiety, elation/euphoria, apathy/indifference, disinhibition, irritability/lability, and aberrant motor behavior. The total score is calculated by adding the scores of the domains (each domain scores ranges from 0 to 12). The total score ranges from 0 to 120 with higher scores indicating greater behavioral impairment.

The mean on the change from baseline at Day 104 was −3.91 (±0.900) for TB006 and 1.60 (±0.853), or a difference of −2.32 favoring TB006. The MMRM analysis result did not reach statistical significance. The point estimate at the Day 104 visit was −3.5 for TB006 and −1.1 for placebo, a difference (2.4 points) favoring TB006. The analysis at Day 36 showed a mean change of −2.91 (±0.948) for TB006 vs 0.87 (±0.920), a difference of −2.03 points favoring TB006. This difference was not significant (p=0.12).

### Example 23: Anti-Gal3 Antibodies Effect on CDR Scores

The longitudinal effect of TB006 on cognition was assessed in subjects undergoing a phase 1b/2a, multi-dose, multi-center study to assess the safety, tolerability, pharmacokinetics, pharmacodynamics, and efficacy of TB006 in patients with Alzheimer's Disease according to the protocol of Example 13, above.

Patients performed a battery of cognition and quality of life tests, and biomarker testing, including the Clinical Dementia Rating Scale-Sum of Boxes (CDR-SB), the Mini Mental State Examination (MMSE), Cognitive Drug Research system (CDR) battery, and the Neuropsychiatry Inventory (NPI), and the EuroQuality of life (EuroQol) 5-Dimension 5-Level (EQ-5D-5L).

Cognition and biomarker testing was performed in the SoA (FIG. 60). As can be seen in FIG. 60, a MMSE was administered between Day-28 and again at Day-1 to establish a base line, and then again at D15, D36, D64, and D104.

The Cognitive Drug Research System is an automated battery amenable to measuring cognitive impairment in patients with AD. It consists of performance tasks measuring attention, working memory, episodic memory, and executive function. It contains 11 tests and is performed on a tablet-like device.

Patients in the TB006 group performed better than the placebo group on “Continuity of Attention” domain, although not reaching statistical significance at the time points. The most noticeable effect on this measure was observed on the Day 29 visit, where an increase in performance compared to baseline was observed in the TB006 group with a concomitant small decrease in performance compared to baseline in the placebo group (p=0.05 and p=0.06 respectively). The observed increases of performance were mainly due to the Digit Vigilance task.

The TB006 group performed significantly better than the placebo group on the “Power of Attention” at the Day 29 (p=0.02) and Day 36 (p=0.03) visits. These increases in performance were due to the observed increase in performance on all three attentional tasks (Simple Reaction Time, Choice Reaction Time, and Digit Vigilance) speed measures.

Patients receiving TB006 performed better than placebo on “Speed of Memory” domain at Visit 3 (p=0.06). This difference was driven by the Word Recognition, Numeric Working Memory, and Picture recognition speed tasks.

No statistically significant nor clinically meaningful effects were observed between groups on Reaction Time Variability, Quality of Working Memory, Quality of Episodic Memory, Executive Function, Quality of Memory, Overall Accuracy, Overall Speed, and Overall Efficiency.

1-50. (canceled)

51. A method of treating a neurodegenerative disease, the method comprising administering an anti-Gal3 antibody or binding fragment thereof to a subject,
wherein the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the V_{H}-CDR1 comprises an amino acid sequence according to SEQ ID NO: 31;
the V_{H}-CDR2 comprises an amino acid sequence according to SEQ ID NO: 72;
the V_{H}-CDR3 comprises an amino acid sequence according to SEQ ID NO: 113;
the V_{L}-CDR1 comprises an amino acid sequence according to SEQ ID NO: 171;
the V_{L}-CDR2 comprises an amino acid sequence according to SEQ ID NO: 222; and
the V_{L}-CDR3 comprises an amino acid sequence an amino acid sequence according to SEQ ID NOs: 249.

52. The method of claim 51, wherein the neurodegenerative disease comprises Parkinson's disease, Alzheimer's disease, or dementia.

53. The method of claim 51, wherein the method further comprises identifying a subject as having or likely having Alzheimer's disease, Parkinson's disease, and/or dementia.

54. The method of claim 51, wherein the method further comprises performing a Mini Mental State Examination (MMSE), EQ-5D-5L, Neuropsychiatric Inventory (NPI), Clinical Dementia Rating Scale-Sum of Boxes (CDR-SB), Cognitive Drug Research battery, an assessment of strength, locomotor function and/or balance, an assessment of the frequency and/or severity of symptoms related to aging and/or aging-related senescence, an assessment of whole brain volume, an assessment of hippocampal brain volume, an assessment of brain atrophy, one or more standard cognitive assessments, performing one or more PET scans and/or MRI scans on the subject, and/or monitoring one or more of the subject's biomarkers selected from the list consisting of:
a biomarker for Parkinson's disease, Alzheimer's disease, dementia, inflammation, autoimmune disorders, diabetes, cardiovascular disease, organ dysfunction, organ damage, kidney dysfunction, kidney damage, liver dysfunction, liver damage, heart dysfunction, heart damage, heart failure, bone disorder, anemia, hematopoietic disorders, metabolic disease, hypercoagulable state, cancer, cancer progression, tumor formation, tumor progression, prothrombotic state in cancer and other acute phases, immune deficiency, morbidity, mortality, cell stress, DNA/protein damage, neurodegenerative disease, cellular senescence, fibrosis, frailty, chronological age, biological age, physical capability, strength, locomotor function, balance, brain function, body composition, cardiovascular system, or respiratory system, telomere length (TL), average TL, TL structure, shortest TL, Reactive Oxygen Species (ROS), mitochondrial dysfunction, autophagy, telomerase activity, gut microbiome, a-Klotho, adiponectin, sirtuin 1 (SIRT1), growth differentiation factor 15 (GDF15), sirtuin 6 (SIRT6), growth differentiation factor 11 (GDF11), skin microbiome, microRNA (miRNA), extracellular RNA (exRNA), grip strength, walking speed, standing balance, timed up and go test, atherosclerotic lesions, muscle mass, systolic blood pressure, cognitive function, body mass index, bone density, lung function, waist circumference, health assessments, lymphocytes, white blood cells, C-reactive protein (CRP/hsCRP), interleukins, IL6, tumor necrosis factor alpha (TNFα), monocytes, IL8, IL15, IL1-β, transforming growth factor beta (TGF-β), CXCL1, insulin, glucose, fasted glucose, glycated hemoglobin (HbA1c), C-peptide, extracellular vesicles (EVs), plasminogen activator inhibitor-1 (PAI1), cholesterol, LDL-cholesterol, triglycerides, HDL-cholesterol, atherosclerosis, blood pressure, albumin, creatinine, cystatin c, urea, alkaline phosphatase, bilirubin, hemoglobin, hematocrit, RBC, mean corpuscular volume (MCV), mean corpuscular hemoglobin concentration (MCHC), plasma Aβ40, Aβ42, tau (phosphorylated), NFL, NFH, and/or Gal3, or any combination thereof.

55. The method of claim 51, wherein the anti-Gal3 antibody or binding fragment thereof is administered at a dose of at least 70 mg or at least 0.1 mg/kg.

56. The method of claim 51, wherein the anti-Gal3 antibody or binding fragment thereof is administered to the subject by IV infusion over a course of at least 10-200 minutes.

57. The method of claim 51, wherein the method further comprises administration of an anti-infective, antibiotic, corticosteroid, opioid analgesic, anxiolytic, muscle relaxant, paracetamol, acetaminophen, or any combination thereof.

58. The method of claim 51, wherein the anti-Gal3 antibody or binding fragment thereof is administered to the subject at least once weekly for 5 or more weeks or at least once monthly for one or more months.

59. The method of claim 51, wherein the anti-Gal3 antibody or binding fragment thereof is administered at least 1-12 times in a month via intravenous infusion for a total administered dose of 140-4000 mg or 0.1-300 mg/kg.

60. The method of claim 51, further comprising administration of the anti-Gal3 antibody or binding fragment thereof at least once monthly for a total administered dose of 140-4000 mg or 0.1-300 mg/kg.

61. The method of claim 51, wherein the subject's CDR-SB score, mean global CDR-SB score, mean Memory CDR-SB subdomain score, mean Orientation CDR-SB subdomain score, mean Judgement and Problem Solving CDR-SB subdomain score, mean Community Affairs CDR-SB subdomain score, mean Home and Hobbies CDR-SB subdomain score, mean Personal Care CDR-SB subdomain score, or any combination thereof, is decreased by at least 0.5 points; and/or the subject's MMSE score is increased by at least 1 point following administration of the anti-Gal3 antibody or binding fragment thereof.

62. The method of claim 51, wherein the anti-Gal3 antibody or binding fragment thereof is administered as a therapeutic formulation comprising the anti-Gal3 antibody or binding fragment thereof, Histidine, Methionine, NaCl, and a polysorbate.

63. The method of claim 51, wherein the anti-Gal3 antibody or binding fragment thereof is administered as a therapeutic formulation comprising 20 mg/mL of the anti-Gal3 antibody or binding fragment thereof, 20 mM L-Histidine, 5 mM Methionine, 100 mM NaCl, 0.02% PS80, and a pH of 5.8.

64. The method of claim 63, wherein the therapeutic formulation is administered 5 times in a month via intravenous infusion over the course of 1 hour for a total administered dose of 140-4000 mg, followed by administration once monthly, every 28 days, via intravenous infusion over the course of 1 hour for a total administered dose of 140-4000 mg.

65. The method of claim 51, wherein the anti-Gal3 antibody or binding fragment thereof is administered as a therapeutic formulation comprising 20 mg/mL of the anti-Gal3 antibody or binding fragment thereof, 20 mM L-Histidine, 5 mM Methionine, 100 mM NaCl, 0.02% PS80, and a pH of 5.8, wherein the therapeutic formulation is administered 5 times in a month via intravenous infusion over the course of 1 hour for a total administered dose of 140-4000 mg, followed by administration once monthly, every 28 days, via intravenous infusion over the course of 1 hour for a total administered dose of 140-4000 mg.

66. The method of claim 51, wherein the anti-Gal3 antibody or binding fragment thereof comprises a heavy chain variable region comprising an amino acid sequence having at least 90% identity to a sequence according to any one of SEQ ID NO: 298 or 309-311.

67. The method of claim 51, wherein the anti-Gal3 antibody or binding fragment thereof comprises a light chain variable region comprising an amino acid sequence having at least 90% identity to a sequence according to any one of SEQ ID NO: 375, 387 or 388.

68. The method of claim 51, wherein the anti-Gal3 antibody or binding fragment thereof light chain comprises an amino acid sequence having at least 90% identity to an amino acid sequence selected from SEQ ID NO: 496 or 508-510.

69. A method of treating a neurodegenerative disease, the method comprising administering an anti-Gal3 antibody or binding fragment thereof to a subject,
wherein the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein the V_{H}-CDR1 comprises an amino acid sequence according to SEQ ID NO: 31;
the V_{H}-CDR2 comprises an amino acid sequence according to SEQ ID NO: 72;
the V_{H}-CDR3 comprises an amino acid sequence according to SEQ ID NO: 113;
the V_{L}-CDR1 comprises an amino acid sequence according to SEQ ID NO: 171;
the V_{L}-CDR2 comprises an amino acid sequence according to SEQ ID NO: 222; and
the V_{L}-CDR3 comprises an amino acid sequence an amino acid sequence according to SEQ ID NOs: 249;
wherein the anti-Gal3 antibody or binding fragment thereof is administered as a therapeutic formulation,
wherein the therapeutic formulation is administered at least 1-12 times in a month via intravenous infusion for a total administered dose of 140-4000 mg or 0.1-300 mg/kg.

70. A method of treating a neurodegenerative disease, the method comprising administering an anti-Gal3 antibody or binding fragment thereof to a subject,
wherein the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the V_{H}-CDR1 comprises an amino acid sequence according to SEQ ID NO: 31;
the V_{H}-CDR2 comprises an amino acid sequence according to SEQ ID NO: 72;
the V_{H}-CDR3 comprises an amino acid sequence according to SEQ ID NO: 113;
the V_{L}-CDR1 comprises an amino acid sequence according to SEQ ID NO: 171;
the V_{L}-CDR2 comprises an amino acid sequence according to SEQ ID NO: 222; and
the V_{L}-CDR3 comprises an amino acid sequence an amino acid sequence according to SEQ ID NOs: 249;
wherein the anti-Gal3 antibody or binding fragment thereof is administered as a therapeutic formulation,
wherein the therapeutic formulation comprises the anti-Gal3 antibody or binding fragment thereof, Histidine, Methionine, NaCl, and a polysorbate;
wherein the therapeutic formulation is administered at least 1-12 times in a month via intravenous infusion for a total administered dose of 140-4000 mg or 0.1-300 mg/kg.
